(19) United States
(12) Patent Application Publication
(10) Pub. No.: US 2025/0197423 A1
(43) Pub. Date: Jun. 19, 2025

(54) RAS INHIBITORS

(71) Applicant: Revolution Medicines, Inc., Redwood City, CA (US)

(72) Inventors: Elena S. KOLTUN, Foster City, CA (US); James CREGG, Belmont, CA (US); Adrian L. GILL, Atherton, CA (US); John E. KNOX, Emerald Hills, CA (US); Yang LIU, Foster City, CA (US); G. Leslie BURNETT, Redwood City, CA (US)

(21) Appl. No.: 18/769,009

(22) Filed: Jul. 10, 2024

Related U.S. Application Data
(63) Continuation of application No. PCT/US2023/060288, filed on Jan. 9, 2023.
(60) Provisional application No. 63/298,098, filed on Jan. 10, 2022.

(51) Int. Cl. C07D 519/00; A61K 31/504; A61K 31/5383; A61K 31/5386; A61K 31/547; A61P 35/00; C07D 498/22; C07D 513/22

(52) U.S. Cl. CPC C07D 519/00; A61K 31/504; A61K 31/5383; A61K 31/5386; A61K 31/547; A61P 35/00; C07D 498/22; C07D 513/22

(57) ABSTRACT

The disclosure features macrocyclic compounds, and pharmaceutical compositions and protein complexes thereof, capable of inhibiting Ras proteins, and their uses in the treatment of cancers.

### BACKGROUND

The vast majority of small molecule drugs act by binding a functionally important pocket on a target protein, thereby modulating the activity of that protein. For example, cholesterol-lowering drugs known as statins bind the enzyme active site of HMG-COA reductase, thus preventing the enzyme from engaging with its substrates. The fact that many such drug/target interacting pairs are known may have misled some into believing that a small molecule modulator could be discovered for most, if not all, proteins provided a reasonable amount of time, effort, and resources. This is far from the case. Current estimates are that only about 10% of all human proteins are targetable by small molecules. Bojadzic and Buchwald, Curr Top Med Chem 18:674-699 (2019). The other 90% are currently considered refractory or intractable toward above-mentioned small molecule drug discovery. Such targets are commonly referred to as “undruggable.” These undruggable targets include a vast and largely untapped reservoir of medically important human proteins. Thus, there exists a great deal of interest in discovering new molecular modalities capable of modulating the function of such undruggable targets.

It has been well established in literature that Ras proteins (K-Ras, H-Ras, and N-Ras) play an essential role in various human cancers and are therefore appropriate targets for anticancer therapy. Indeed, mutations in Ras proteins account for approximately 30% of all human cancers in the United States, many of which are fatal. Dysregulation of Ras proteins by activating mutations, overexpression or upstream activation is common in human tumors, and activating mutations in Ras are frequently found in human cancer. For example, activating mutations at codon 12 in Ras proteins function by inhibiting both GTPase-activating protein (GAP)-dependent and intrinsic hydrolysis rates of GTP, significantly skewing the population of Ras mutant proteins to the “on” (GTP-bound) state (Ras (ON)), leading to oncogenic MAPK signaling. Notably, Ras exhibits a picomolar affinity for GTP, enabling Ras to be activated even in the presence of low concentrations of this nucleotide. Mutations at codons 13 (e.g., G13C) and 61 (e.g., Q61K) of Ras are also responsible for oncogenic activity in some cancers.

Despite extensive drug discovery efforts against Ras during the last several decades, only two agents targeting the K-Ras G12C mutant have been approved in the U.S. (sotorasib and adagrasib). Additional efforts are needed to uncover additional medicines for cancers driven by the various Ras mutations.

### SUMMARY

Provided herein are Ras inhibitors. The approach described herein entails formation of a high affinity three-component complex, or conjugate, between a synthetic ligand and two intracellular proteins which do not interact under normal physiological conditions: the target protein of interest (e.g., Ras), and a widely expressed cytosolic chaperone (presenter protein) in the cell (e.g., cyclophilin A). More specifically, in some embodiments, the inhibitors of Ras described herein induce a new binding pocket in Ras by driving formation of a high affinity tri-complex, or conjugate, between the Ras protein and the widely expressed cytosolic chaperone, cyclophilin A (CYPA). Without being bound by theory, the inventors believe that one way the inhibitory effect on Ras is effected by compounds of the invention and the complexes, or conjugates, they form is by steric occlusion of the interaction site between Ras and downstream effector molecules, such as RAF and PI3K, which are required for propagating the oncogenic signal.

As such, in some embodiments, the disclosure features a compound, or pharmaceutically acceptable salt thereof, of structural Formula I:

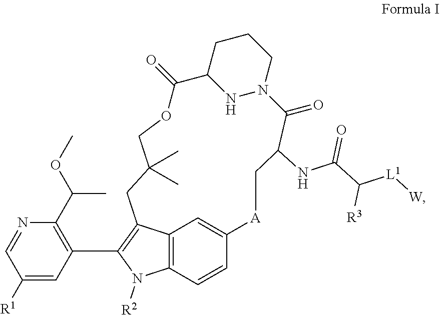

wherein A is optionally substituted 3 to 6-membered heterocycloalkylene, optionally substituted 3 to 6-membered cycloalkylene, optionally substituted 6-membered arylene, or optionally substituted 5 to 10-membered heteroarylene;
L¹ is absent or a linker;
W is a cross-linking group comprising a vinyl ketone, vinyl sulfone, ynone, or an alkynyl sulfone;
R¹ is hydrogen, optionally substituted 3 to 10-membered heterocycloalkyl, or optionally substituted C₁-C₆ heteroalkyl;
R² is optionally substituted C₁-C₆ alkyl; and
R³ is optionally substituted C₁-C₆ alkyl or optionally substituted C₁-C₃ heteroalkyl.

Also provided are pharmaceutical compositions comprising a compound of Formula I, or a pharmaceutically acceptable salt thereof, and a pharmaceutically acceptable excipient. Also provided are pharmaceutical compositions comprising a compound of Table 1, or a pharmaceutically acceptable salt thereof, and a pharmaceutically acceptable excipient.

Also provided is a method of treating cancer in a subject in need thereof, the method comprising administering to the subject a therapeutically effective amount of a compound of the present invention, or a pharmaceutically acceptable salt thereof.

In some embodiments, a method is provided of treating a Ras protein-related disorder in a subject in need thereof, the method comprising administering to the subject a therapeutically effective amount of a compound of the present invention, or a pharmaceutically acceptable salt thereof.

Further provided is a method of inhibiting a Ras protein in a cell, the method comprising contacting the cell with an effective amount of a compound of the present invention, or a pharmaceutically acceptable salt thereof.

It is specifically contemplated that any limitation discussed with respect to one embodiment of the invention may apply to any other embodiment of the invention. Furthermore, any compound or composition of the invention may be used in any method of the invention, and any method of the invention may be used to produce or to utilize any compound or composition of the invention.

### Definitions and Chemical Terms

In this application, unless otherwise clear from context, (i) the term “a” means “one or more”; (ii) the term “or” is used to mean “and/or” unless explicitly indicated to refer to alternatives only or the alternative are mutually exclusive, although the disclosure supports a definition that refers to only alternatives and “and/or”; (iii) the terms “comprising” and “including” are understood to encompass itemized components or steps whether presented by themselves or together with one or more additional components or steps; and (iv) where ranges are provided, endpoints are included.

As used herein, the term “about” is used to indicate that a value includes the standard deviation of error for the device or method being employed to determine the value. In certain embodiments, the term “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of a stated value, unless otherwise stated or otherwise evident from the context (e.g., where such number would exceed 100% of a possible value).

As used herein, the term “adjacent” in the context of describing adjacent atoms refers to bivalent atoms that are directly connected by a covalent bond.

A “compound of the present invention” and similar terms as used herein, whether explicitly noted or not, refers to Ras inhibitors described herein, including compounds of Formula I and subformula thereof, for example, a compound of Table 1, as well as salts (e.g., pharmaceutically acceptable salts), solvates, hydrates, stereoisomers (including atropisomers), and tautomers thereof.

The term “wild-type” refers to an entity having a structure or activity as found in nature in a “normal” (as contrasted with mutant, diseased, altered, etc) state or context. Those of ordinary skill in the art will appreciate that wild-type genes and polypeptides often exist in multiple different forms (e.g., alleles).

Those skilled in the art will appreciate that certain compounds described herein can exist in one or more different isomeric (e.g., stereoisomers, geometric isomers, atropisomers, tautomers) or isotopic (e.g., in which one or more atoms has been substituted with a different isotope of the atom, such as hydrogen substituted for deuterium) forms. Unless otherwise indicated or clear from context, a depicted structure can be understood to represent any such isomeric or isotopic form, individually or in combination.

Compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

In some embodiments, one or more compounds depicted herein may exist in different tautomeric forms. As will be clear from context, unless explicitly excluded, references to such compounds encompass all such tautomeric forms. In some embodiments, tautomeric forms result from the swapping of a single bond with an adjacent double bond and the concomitant migration of a proton. In certain embodiments, a tautomeric form may be a prototropic tautomer, which is an isomeric protonation states having the same empirical formula and total charge as a reference form. Examples of moieties with prototropic tautomeric forms are ketone-enol pairs, amide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. In some embodiments, tautomeric forms can be in equilibrium or sterically locked into one form by appropriate substitution. In certain embodiments, tautomeric forms result from acetal interconversion.

Unless otherwise stated, structures depicted herein are also meant to include compounds that differ only in the presence of one or more isotopically enriched atoms. Exemplary isotopes that can be incorporated into compounds of the present invention include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Isotopically labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁸F are useful for positron emission tomography (PET) studies to examine substrate receptor occupancy. Preparations of isotopically labelled compounds are known to those of skill in the art. For example, isotopically labeled compounds can generally be prepared by following procedures analogous to those disclosed for compounds of the present invention described herein, by substituting an isotopically labeled reagent for a non-isotopically labeled reagent.

Non-limiting examples of moeities that may contain one or more deuterium substitutions in compounds of the present invention, where any position “R” may be deuterium (D), include

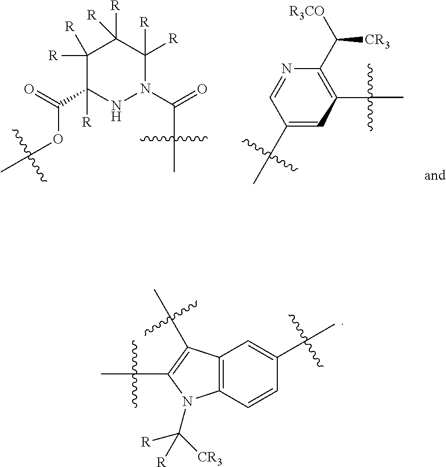

Additional examples include moieties such as

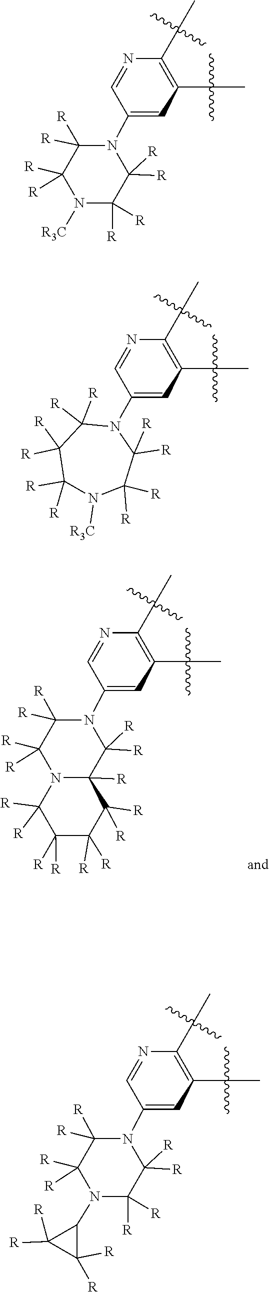

and deuteration of similar R¹-type moieties, wherein the definition of R¹ is found herein (e.g., in compounds of Formula I, Ia, II-5, II-5a, II-6, II-6a, II-6b, and II-6c). Deuteration of moieties within substituent W in compounds of the present invention are also contemplated, where W is defined herein (see, e.g., generic Formulas I and II and subformulas thereof as well as specific examples of W described herein, such as

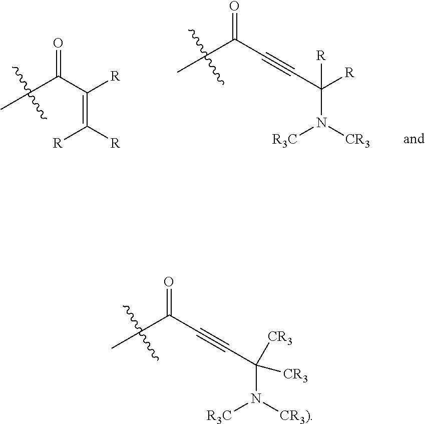

Moreover, deuteration of available positions in any A moiety of compounds of the Formulas described herein is also contemplated, such as

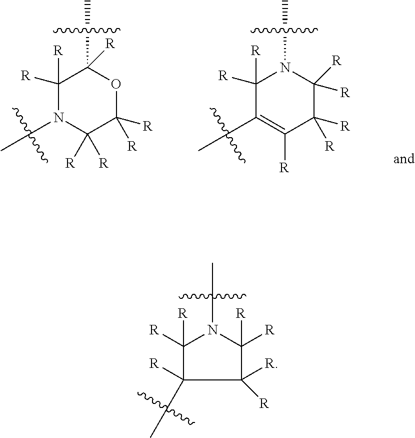

Further, deuterium substitution may also take place in compounds of the present invention at the linker position, such as

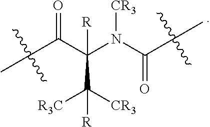

In a further embodiment, silylation substitution is also contemplated, such as in the linker as follows:

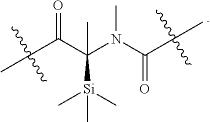 [SMILES: *C(=O)N(C)[C@@](C)(C(*)=O)[Si](C)(C)C]

As is known in the art, many chemical entities can adopt a variety of different solid forms such as, for example, amorphous forms or crystalline forms (e.g., polymorphs, hydrates, solvate). In some embodiments, compounds of the present invention may be utilized in any such form, including in any solid form. In some embodiments, compounds described or depicted herein may be provided or utilized in hydrate or solvate form.

At various places in the present specification, substituents of compounds of the present disclosure are disclosed in groups or in ranges. It is specifically intended that the present disclosure include each and every individual subcombination of the members of such groups and ranges. For example, the term “C₁-C₆ alkyl” is specifically intended to individually disclose methyl, ethyl, C₃ alkyl, C₄ alkyl, C₅ alkyl, and C₆ alkyl. Furthermore, where a compound includes a plurality of positions at which substituents are disclosed in groups or in ranges, unless otherwise indicated, the present disclosure is intended to cover individual compounds and groups of compounds (e.g., genera and subgenera) containing each and every individual subcombination of members at each position.

The term “optionally substituted X” (e.g., “optionally substituted alkyl”) is intended to be equivalent to “X, wherein X is optionally substituted” (e.g., “alkyl, wherein said alkyl is optionally substituted”). It is not intended to mean that the feature “X” (e.g., alkyl) per se is optional. As described herein, certain compounds of interest may contain one or more “optionally substituted” moieties. In general, the term “substituted”, whether preceded by the term “optionally” or not, means that one or more hydrogens of the designated moiety are replaced with a suitable substituent, e.g., any of the substituents or groups described herein. Unless otherwise indicated, an “optionally substituted” group may have a suitable substituent at each substitutable position of the group, and when more than one position in any given structure may be substituted with more than one substituent selected from a specified group, the substituent may be either the same or different at every position. For example, in the term “optionally substituted C₁-C₆ alkyl-C₂-C₉ heteroaryl,” the alkyl portion, the heteroaryl portion, or both, may be optionally substituted. Combinations of substituents envisioned by the present disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group may be, independently, deuterium; halogen; —(CH₂)₀₋₄R^{∘}; —(CH₂)₀₋₄OR^{∘}; —O(CH₂)₀₋₄R^{∘}; —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; 4-8 membered saturated or unsaturated heterocycloalkyl (e.g., pyridyl); 3-8 membered saturated or unsaturated cycloalkyl (e.g., cyclopropyl, cyclobutyl, or cyclopentyl); —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘}N(R^{∘})C(O)R^{∘}; —N(R^{∘}N(R^{∘})C(O)NR^{∘})₂; —N(R^{∘}N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄—C(O)—N(R^{∘})₂; —(CH₂)₀₋₄—C(O)—N(R^{∘})—S(O)₂—R^{∘}; —C(NCN)NR^{∘}₂; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄S R^{∘}; —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(O) R^{o}R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NOR^{∘})NR^{∘}₂; —C(NH)NR^{∘}₂; —P(O)₂R^{∘}; —P(O)R^{∘}₂; —P(O)(OR^{∘})₂; —OP(O)R^{∘})₂; —OP(O)(OR^{∘})₂; —OP(O)(OR^{∘})R^{∘}, —SiR^{∘}₃; —(C₁₋₄ straight or branched alkylene) O—N(R^{∘})₂; or —(C₁₋₄ straight or branched) alkylene) C(O)O—N(R^{∘})₂, wherein each R^{∘} may be substituted as defined below and is independently hydrogen, —C₁₋₆ aliphatic, —CH₂Ph, —O(CH₂)₀₋₁Ph, —CH₂-(5-6 membered heteroaryl ring), or a 3-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), may be, independently, halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{•} include ═O and ═S.

Suitable divalent substituents on a saturated carbon atom of an “optionally substituted” group include the following: ═O, ═S, ═NNR^{*}₂, ═NNHC(O)R^{*}, ═NNHC(O)OR^{*}, =NNHS(O)₂R^{*}, ═NR^{*}, ═NOR^{*}, —O(C(R*₂))₂₋₃O—, or —S(C(R^{*}₂))₂₋₃S—, wherein each independent occurrence of R^{*} is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 3-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on an aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{†} include ═O and ═S.

The term “acetyl,” as used herein, refers to the group —C(O)CH₃.

The term “alkoxy,” as used herein, refers to a —O—C₁-C₂₀ alkyl group, wherein the alkoxy group is attached to the remainder of the compound through an oxygen atom.

The term “alkyl,” as used herein, refers to a saturated, straight or branched monovalent hydrocarbon group containing from 1 to 20 (e.g., from 1 to 10 or from 1 to 6) carbons. In some embodiments, an alkyl group is unbranched (i.e., is linear); in some embodiments, an alkyl group is branched. Alkyl groups are exemplified by, but not limited to, methyl, ethyl, n- and iso-propyl, n-, sec-, iso- and tert-butyl, and neopentyl.

The term “alkylene,” as used herein, represents a saturated divalent hydrocarbon group derived from a straight or branched chain saturated hydrocarbon by the removal of two hydrogen atoms, and is exemplified by methylene, ethylene, isopropylene, and the like. The term “Cₓ-C_{y} alkylene” represents alkylene groups having between x and y carbons. Exemplary values for x are 1, 2, 3, 4, 5, and 6, and exemplary values for y are 2, 3, 4, 5, 6, 7, 8, 9, 10, 12, 14, 16, 18, or 20 (e.g., C₁-C₆, C₁-C₁₀, C₂-C₂₀, C₂-C₆, C₂-C₁₀, or C₂-C₂₀ alkylene). In some embodiments, the alkylene can be further substituted with 1, 2, 3, or 4 substituent groups as defined herein.

The term “alkenyl,” as used herein, represents monovalent straight or branched chain groups of, unless otherwise specified, from 2 to 20 carbons (e.g., from 2 to 6 or from 2 to 10 carbons) containing one or more carbon-carbon double bonds and is exemplified by ethenyl, 1-propenyl, 2-propenyl, 2-methyl-1-propenyl, 1-butenyl, and 2-butenyl. Alkenyls include both cis and trans isomers. The term “alkenylene,” as used herein, represents a divalent straight or branched chain groups of, unless otherwise specified, from 2 to 20 carbons (e.g., from 2 to 6 or from 2 to 10 carbons) containing one or more carbon-carbon double bonds.

The term “alkynyl,” as used herein, represents monovalent straight or branched chain groups from 2 to 20 carbon atoms (e.g., from 2 to 4, from 2 to 6, or from 2 to 10 carbons) containing a carbon-carbon triple bond and is exemplified by ethynyl, and 1-propynyl.

The term “alkynyl sulfone.” as used herein, represents a group comprising the structure

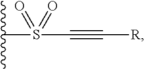

wherein R is any chemically feasible substituent described herein.

The term “amino,” as used herein, represents —N(R^{\})₂, e.g., —NH₂ and —N(CH₃)₂.

The term “aminoalkyl,” as used herein, represents an alkyl moiety substituted on one or more carbon atoms with one or more amino moieties.

The term “amino acid,” as described herein, refers to a molecule having a side chain, an amino group, and an acid group (e.g., —CO₂H or —SO₃H), wherein the amino acid is attached to the parent molecular group by the side chain, amino group, or acid group (e.g., the side chain). As used herein, the term “amino acid” in its broadest sense, refers to any compound or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. Exemplary amino acids include alanine, arginine, asparagine, aspartic acid, cysteine, glutamic acid, glutamine, glycine, histidine, optionally substituted hydroxylnorvaline, isoleucine, leucine, lysine, methionine, norvaline, ornithine, phenylalanine, proline, pyrrolysine, selenocysteine, serine, taurine, threonine, tryptophan, tyrosine, and valine.

The term “aryl,” as used herein, represents a monovalent monocyclic, bicyclic, or multicyclic ring system formed by carbon atoms, wherein the ring attached to the pendant group is aromatic. Examples of aryl groups are phenyl, naphthyl, phenanthrenyl, and anthracenyl. An aryl ring can be attached to its pendant group at any heteroatom or carbon ring atom that results in a stable structure and any of the ring atoms can be optionally substituted unless otherwise specified.

The term “C₀,” as used herein, represents a bond. For example, part of the term —N(C(O)—(C₀-C₅ alkylene-H)— includes-N(C(O)—(C₀ alkylene-H)—, which is also represented by —N(C(O)—H)—.

The terms “carbocyclic” and “carbocyclyl,” as used herein, refer to a monovalent, optionally substituted C₃-C₁₂ monocyclic, bicyclic, or tricyclic ring structure, which may be bridged, fused or spirocyclic, in which all the rings are formed by carbon atoms and at least one ring is non-aromatic. Carbocyclic structures include cycloalkyl, cycloalkenyl, and cycloalkynyl groups. Examples of carbocyclyl groups are cyclohexyl, cyclohexenyl, cyclooctynyl, 1,2-dihydronaphthyl, 1,2,3,4-tetrahydronaphthyl, fluorenyl, indenyl, indanyl, decalinyl, and the like. A carbocyclic ring can be attached to its pendant group at any ring atom that results in a stable structure and any of the ring atoms can be optionally substituted unless otherwise specified.

The term “carbonyl,” as used herein, represents a C(O) group, which can also be represented as C═O.

The term “carboxyl,” as used herein, means —CO₂H, (C═O) (OH), COOH, or C(O)OH or the unprotonated counterparts.

The term “cyano,” as used herein, represents a-CN group.

The term “cycloalkyl,” as used herein, represents a monovalent saturated cyclic hydrocarbon group, which may be bridged, fused or spirocyclic having from three to eight ring carbons, unless otherwise specified, and is exemplified by cyclopropyl, cyclobutyl, cyclopentyl, cyclohexyl, cycloheptyl, and cycloheptyl.

The term “cycloalkenyl,” as used herein, represents a monovalent, non-aromatic, saturated cyclic hydrocarbon group, which may be bridged, fused or spirocyclic having from three to eight ring carbons, unless otherwise specified, and containing one or more carbon-carbon double bonds.

The term “diastereomer,” as used herein, means stereoisomers that are not mirror images of one another and are non-superimposable on one another.

The term “enantiomer,” as used herein, means each individual optically active form of a compound of the invention, having an optical purity or enantiomeric excess (as determined by methods standard in the art) of at least 80% (i.e., at least 90% of one enantiomer and at most 10% of the other enantiomer), preferably at least 90% and more preferably at least 98%.

The term “guanidinyl,” refers to a group having the structure:

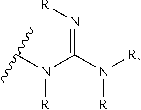

wherein each R is, independently, any any chemically feasible substituent described herein.

The term “guanidinoalkyl alkyl,” as used herein, represents an alkyl moiety substituted on one or more carbon atoms with one or more guanidinyl moieties.

The term “haloacetyl,” as used herein, refers to an acetyl group wherein at least one of the hydrogens has been replaced by a halogen.

The term “haloalkyl,” as used herein, represents an alkyl moiety substituted on one or more carbon atoms with one or more of the same of different halogen moieties.

The term “halogen,” as used herein, represents a halogen selected from bromine, chlorine, iodine, or fluorine.

The term “heteroalkyl,” as used herein, refers to an “alkyl” group, as defined herein, in which at least one carbon atom has been replaced with a heteroatom (e.g., an O, N, or S atom). The heteroatom may appear in the middle or at the end of the radical.

The term “heteroaryl,” as used herein, represents a monovalent, monocyclic, or polycyclic ring structure that contains at least one fully aromatic ring: i.e., they contain 4n+2 pi electrons within the monocyclic or polycyclic ring system and contains at least one ring heteroatom selected from N, O, or S in that aromatic ring. Exemplary unsubstituted heteroaryl groups are of 1 to 12 (e.g., 1 to 11, 1 to 10, 1 to 9, 2 to 12, 2 to 11, 2 to 10, or 2 to 9) carbons. The term “heteroaryl” includes bicyclic, tricyclic, and tetracyclic groups in which any of the above heteroaromatic rings is fused to one or more, aryl or carbocyclic rings, e.g., a phenyl ring, or a cyclohexane ring. Examples of heteroaryl groups include, but are not limited to, pyridyl, pyrazolyl, benzooxazolyl, benzoimidazolyl, benzothiazolyl, imidazolyl, thiazolyl, quinolinyl, tetrahydroquinolinyl, and 4-azaindolyl. A heteroaryl ring can be attached to its pendant group at any ring atom that results in a stable structure and any of the ring atoms can be optionally substituted unless otherwise specified. In some embodiment, the heteroaryl is substituted with 1, 2, 3, or 4 substituents groups.

The term “heterocycloalkyl,” as used herein, represents a monovalent monocyclic, bicyclic, or polycyclic ring system, which may be bridged, fused or spirocyclic, wherein at least one ring is non-aromatic and wherein the non-aromatic ring contains one, two, three, or four heteroatoms independently selected from the group consisting of nitrogen, oxygen, and sulfur. The 5-membered ring has zero to two double bonds, and the 6- and 7-membered rings have zero to three double bonds. Exemplary unsubstituted heterocycloalkyl groups are of 1 to 12 (e.g., 1 to 11, 1 to 10, 1 to 9, 2 to 12, 2 to 11, 2 to 10, or 2 to 9) carbons. The term “heterocycloalkyl” also represents a heterocyclic compound having a bridged multicyclic structure in which one or more carbons or heteroatoms bridges two non-adjacent members of a monocyclic ring, e.g., a quinuclidinyl group. The term “heterocycloalkyl” includes bicyclic, tricyclic, and tetracyclic groups in which any of the above heterocyclic rings is fused to one or more aromatic, carbocyclic, heteroaromatic, or heterocyclic rings, e.g., an aryl ring, a cyclohexane ring, a cyclohexene ring, a cyclopentane ring, a cyclopentene ring, a pyridine ring, or a pyrrolidine ring. Examples of heterocycloalkyl groups are pyrrolidinyl, piperidinyl, 1,2,3,4-tetrahydroquinolinyl, decahydroquinolinyl, dihydropyrrolopyridine, and decahydronapthyridinyl. A heterocycloalkyl ring can be attached to its pendant group at any ring atom that results in a stable structure and any of the ring atoms can be optionally substituted unless otherwise specified.

The term “hydroxy,” as used herein, represents a-OH group.

The term “hydroxyalkyl,” as used herein, represents an alkyl moiety substituted on one or more carbon atoms with one or more-OH moieties.

The term “isomer,” as used herein, means any tautomer, stereoisomer, atropiosmer, enantiomer, or diastereomer of any compound of the invention. It is recognized that the compounds of the invention can have one or more chiral centers or double bonds and, therefore, exist as stereoisomers, such as double-bond isomers (i.e., geometric E/Z isomers) or diastereomers (e.g., enantiomers (i.e., (+) or (−)) or cis/trans isomers). According to the invention, the chemical structures depicted herein, and therefore the compounds of the invention, encompass all the corresponding stereoisomers, that is, both the stereomerically pure form (e.g., geometrically pure, enantiomerically pure, or diastereomerically pure) and enantiomeric and stereoisomeric mixtures, e.g., racemates. Enantiomeric and stereoisomeric mixtures of compounds of the invention can typically be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

As used herein, the term “linker” refers to a divalent organic moiety connecting a first moiety (e.g., a macrocyclic moiety) to a second moiety (e.g., a cross-linking group). In some embodiments, the linker results in a compound capable of achieving an IC50 of 2 uM or less in the Ras-RAF disruption assay protocol provided in the Examples below, and provided here:
The purpose of this biochemical assay is to measure the ability of test compounds to facilitate ternary complex formation between a nucleotide-loaded Ras isoform and cyclophilin A; the resulting ternary complex disrupts binding to a BRAF^{RBD} construct, inhibiting Ras signaling through a RAF effector.
In assay buffer containing 25 mM HEPES pH 7.3, 0.002% Tween20, 0.1% BSA, 100 mM NaCl and 5 mM MgCl₂, tagless Cyclophilin A, His6-K-Ras-GMPPNP (or other Ras variant), and GST-BRAF^{RBD} are combined in a 384-well assay plate at final concentrations of 25 μM, 12.5 nM and 50 nM, respectively. Compound is present in plate wells as a 10-point 3-fold dilution series starting at a final concentration of 30 uM. After incubation at 25° C. for 3 hours, a mixture of Anti-His Eu-W1024 and anti-GST allophycocyanin is then added to assay sample wells at final concentrations of 10 nM and 50 nM, respectively, and the reaction incubated for an additional 1.5 hours. TR-FRET signal is read on a microplate reader (Ex 320 nm, Em 665/615 nm). Compounds that facilitate disruption of a Ras: RAF complex are identified as those eliciting a decrease in the TR-FRET ratio relative to DMSO control wells.

This assay may be used to assess selectivity as well. In some embodiments, a compound of the present invention is selective for one or more particular Ras mutants (e.g., K-Ras G13C) over other Ras mutants (e.g., K-Ras G12C) or wild-type compared to what is known in the art.

In some embodiments, the linker comprises 20 or fewer linear atoms. In some embodiments, the linker comprises 15 or fewer linear atoms. In some embodiments, the linker comprises 10 or fewer linear atoms. In some embodiments, the linker has a molecular weight of under 500 g/mol. In some embodiments, the linker has a molecular weight of under 400 g/mol. In some embodiments, the linker has a molecular weight of under 300 g/mol. In some embodiments, the linker has a molecular weight of under 200 g/mol. In some embodiments, the linker has a molecular weight of under 100 g/mol. In some embodiments, the linker has a molecular weight of under 50 g/mol.

As used herein, a “monovalent organic moiety” is less than 500 kDa. In some embodiments, a “monovalent organic moiety” is less than 400 kDa. In some embodiments, a “monovalent organic moiety” is less than 300 kDa. In some embodiments, a “monovalent organic moiety” is less than 200 kDa. In some embodiments, a “monovalent organic moiety” is less than 100 kDa. In some embodiments, a “monovalent organic moiety” is less than 50 kDa. In some embodiments, a “monovalent organic moiety” is less than 25 kDa. In some embodiments, a “monovalent organic moiety” is less than 20 kDa. In some embodiments, a “monovalent organic moiety” is less than 15 kDa. In some embodiments, a “monovalent organic moiety” is less than 10 kDa. In some embodiments, a “monovalent organic moiety” is less than 1 kDa. In some embodiments, a “monovalent organic moiety” is less than 500 g/mol. In some embodiments, a “monovalent organic moiety” ranges between 500 g/mol and 500 kDa.

The term “stereoisomer,” as used herein, refers to all possible different isomeric as well as conformational forms which a compound may possess (e.g., a compound of any formula described herein), in particular all possible stereochemically and conformationally isomeric forms, all diastereomers, enantiomers or conformers of the basic molecular structure, including atropisomers. Some compounds of the present invention may exist in different tautomeric forms, all of the latter being included within the scope of the present invention.

The term “sulfonyl,” as used herein, represents an —S(O)₂— group.

The term “thiocarbonyl,” as used herein, refers to a —C(S)— group.

The term “vinyl ketone,” as used herein, refers to a group comprising a carbonyl group directly connected to a carbon-carbon double bond.

The term “vinyl sulfone,” as used herein, refers to a group comprising a sulfonyl group directed connected to a carbon-carbon double bond.

The term “ynone,” as used herein, refers to a group comprising the structure

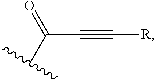

wherein R is any any chemically feasible substituent described herein.

Those of ordinary skill in the art, reading the present disclosure, will appreciate that certain compounds described herein may be provided or utilized in any of a variety of forms such as, for example, salt forms, protected forms, pro-drug forms, ester forms, isomeric forms (e.g., optical or structural isomers), isotopic forms, etc. In some embodiments, reference to a particular compound may relate to a specific form of that compound. In some embodiments, reference to a particular compound may relate to that compound in any form. In some embodiments, for example, a preparation of a single stereoisomer of a compound may be considered to be a different form of the compound than a racemic mixture of the compound; a particular salt of a compound may be considered to be a different form from another salt form of the compound; a preparation containing one conformational isomer ((Z) or (E)) of a double bond may be considered to be a different form from one containing the other conformational isomer ((E) or (Z)) of the double bond; a preparation in which one or more atoms is a different isotope than is present in a reference preparation may be considered to be a different form.

### BRIEF DESCRIPTION OF THE DRAWINGS

FIG. 1A demonstrates selective covalent modification of KRAS^{G13C} by a compound of the present invention, Compound A.

FIG. 1B demonstrates selective covalent modification of KRAS^{G13C} by a compound of the present invention, Compound B. Compound X is a KRAS^{G12C} inhibitor from WO 2021/091982, A647.

FIG. 2 demonstrates single dose PK/PD in vivo inhibition of KRAS^{G13C} (NSCLC CDX KRAS^{G13C/WT} model) using Compound A, a compound of the present invention.

FIG. 3 demonstrates tumor regression in a NSCLC CDX KRAS^{G13C/WT} model using Compound A, a compound of the present invention.

FIG. 4 demonstrates tumor regression in a NSCLC PDX KRAS^{G13C/WT} model using Compound A, a compound of the present invention.

### DETAILED DESCRIPTION

### Compounds

Provided herein are Ras inhibitors. The approach described herein entails formation of a high affinity three-component complex, or conjugate, between a synthetic ligand and two intracellular proteins which do not interact under normal physiological conditions: the target protein of interest (e.g., Ras), and a widely expressed cytosolic chaperone (presenter protein) in the cell (e.g., cyclophilin A). More specifically, in some embodiments, the inhibitors of Ras described herein induce a new binding pocket in Ras by driving formation of a high affinity tri-complex, or conjugate, between the Ras protein and the widely expressed cytosolic chaperone, cyclophilin A (CYPA). Without being bound by theory, the inventors believe that one way the inhibitory effect on Ras is effected by compounds of the invention and the complexes, or conjugates, they form is by steric occlusion of the interaction site between Ras and downstream effector molecules, such as RAF, which are required for propagating the oncogenic signal.

Without being bound by theory, the inventors postulate that both covalent and non-covalent interactions of a compound of the present invention with Ras and the chaperone protein (e.g., cyclophilin A) may contribute to the inhibition of Ras activity. In some embodiments, a compound of the present invention forms a covalent adduct with a side chain of a Ras protein (e.g., a sulfhydryl side chain of the cysteine at position 12 or 13 of a mutant Ras protein). Covalent adducts may also be formed with other side chains of Ras. In addition, or alternatively, non-covalent interactions may be at play: for example, van der Waals, hydrophobic, hydrophilic and hydrogen bond interactions, and combinations thereof, may contribute to the ability of the compounds of the present invention to form complexes and act as Ras inhibitors. Accordingly, a variety of Ras proteins may be inhibited by compounds of the present invention (e.g., K-Ras, N-Ras, H-Ras, and mutants thereof at positions 12, 13 and 61, such as G12C, G12D, G12V, G12S, G13C, G13D, and Q61L, and others described herein).

Methods of determining covalent adduct formation are known in the art. One method of determining covalent adduct formation is to perform a “cross-linking” assay, such as under these conditions.
Note—the following protocol describes a procedure for monitoring cross-linking of K-Ras G12C (GMP-PNP) to a compound of the invention. This protocol may also be executed substituting other Ras proteins or nucleotides, such as G13C.
The purpose of this biochemical assay is to measure the ability of test compounds to covalently label nucleotide-loaded K-Ras isoforms. In assay buffer containing 12.5 mM HEPES PH 7.4, 75 mM NaCl, 1 mM MgCl₂, 1 mM BME, 5 μM Cyclophilin A and 2 μM test compound, a 5 μM stock of GMP-PNP-loaded K-Ras (1-169) G12C is diluted 10-fold to yield a final concentration of 0.5 μM; with final sample volume being 100 μL.
The sample is incubated at 25° C. for a time period of up to 24 hours prior to quenching by the addition of 10 μL of 5% Formic Acid. Quenched samples are centrifuged at 15000 rpm for 15 minutes in a benchtop centrifuge before injecting a 10 μL aliquot onto a reverse phase C4 column and eluting into the mass spectrometer with an increasing acetonitrile gradient in the mobile phase. Analysis of raw data may be carried out using Waters MassLynx MS software, with % bound calculated from the deconvoluted protein peaks for labeled and unlabeled K-Ras.

Accordingly, provided herein is a compound, or pharmaceutically acceptable salt thereof, having the structure of Formula I:

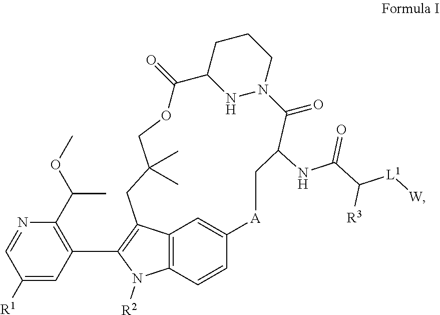

wherein A is optionally substituted 3 to 6-membered heterocycloalkylene, optionally substituted 3 to 6-membered cycloalkylene, optionally substituted 6-membered arylene, or optionally substituted 5 to 10-membered heteroarylene;
L¹ is absent or a linker;
W is a cross-linking group comprising a vinyl ketone, vinyl sulfone, ynone, or an alkynyl sulfone;
R¹ is hydrogen, optionally substituted 3 to 10-membered heterocycloalkyl, or optionally substituted C₁-C₆ heteroalkyl;
R² is optionally substituted C₁-C₆ alkyl; and
R³ is optionally substituted C₁-C₆ alkyl or optionally substituted C₁-C₃ heteroalkyl.

In some embodiments, W is a cross-linking group comprising a vinyl ketone, vinyl sulfone, or an ynone.

In some embodiments, provided herein is a compound, or pharmaceutically acceptable salt thereof, having the structure of Formula Ia:

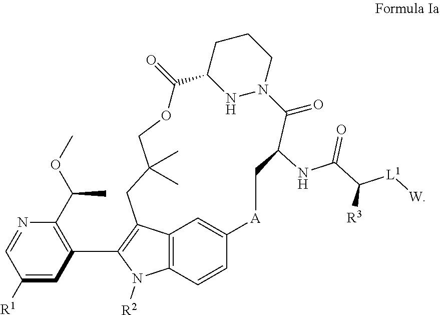

In some embodiments of compounds of the present invention, A is optionally substituted thiazole-diyl, optionally substituted oxazole-diyl, optionally substituted morpholine-diyl, optionally substituted pyrrolidine-diyl, optionally substituted pyridine-diyl, optionally substituted azetidine-diyl, optionally substituted pyrazine-diyl, optionally substituted pyrimidine-diyl, optionally substituted piperidine-diyl, optionally substituted oxadiazole-diyl, optionally substituted thiadiazole-diyl, optionally substituted triazole-diyl, optionally substituted thiomorpholine-diyl, or optionally substituted phenylene.

In some embodiments, the disclosure features a compound, or pharmaceutically acceptable salt thereof, of structural Formula II-1:

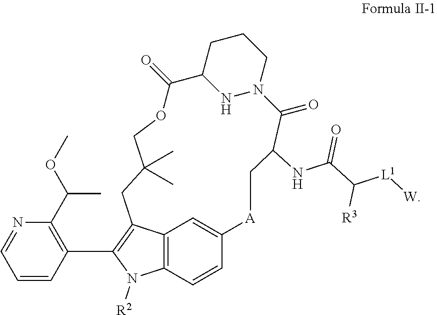

In some embodiments, a compound having the structure of Formula II-2 is provided, or a pharmaceutically acceptable salt thereof:

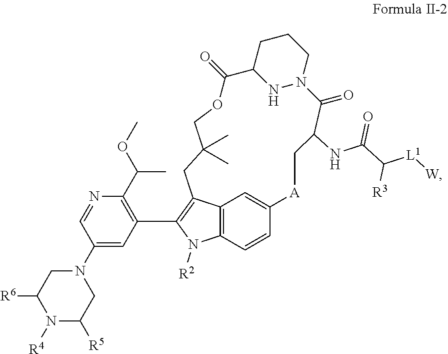

wherein R⁴, R⁵, and R⁶ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered heterocycloalkyl; or
R⁴ and R⁵ combine with the atoms to which they are attached to form an optionally substituted 3 to 8-membered cycloalkyl or an optionally substituted 3 to 8-membered heterocycloalkyl; or
R⁴ and R⁶ combine with the atoms to which they are attached to form an optionally substituted 3 to 8-membered cycloalkyl or an optionally substituted 3 to 8-membered heterocycloalkyl.

In some embodiments, a compound of the present invention has the structure of Formula II-3, or a pharmaceutically acceptable salt thereof:

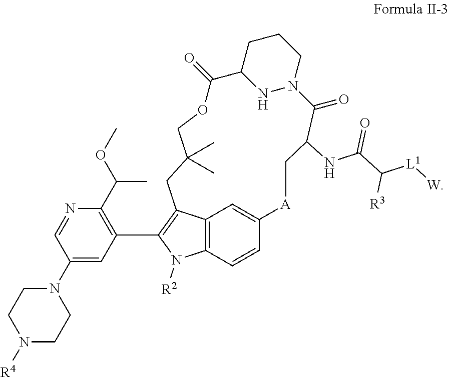

In some embodiments, a compound of the present invention has the structure of Formula II-4, or a pharmaceutically acceptable salt thereof:

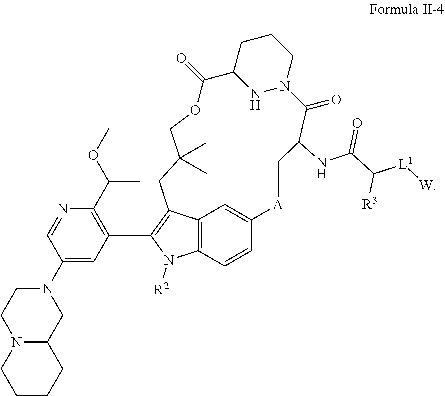

In some embodiments, a compound of the present invention has the structure of Formula II-4b, or a pharmaceutically acceptable salt thereof:

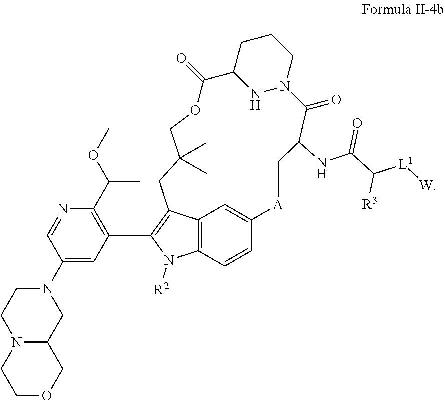

In some embodiments of a compound of the present invention, R² is

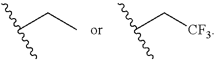

In some embodiments of a compound of the present invention, R³ is optionally substituted C₁-C₆ alkyl. In some embodiments, R³ is:

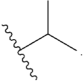 [SMILES: *C(C)C]

In some embodiments of a compound of the present invention, R³ is optionally substituted C₁-C₃ heteroalkyl. In some embodiments, R³ is:

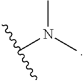 [SMILES: *N(C)C]

In some embodiments of a compound of the present invention, A is optionally substituted 5 to 10-membered heteroarylene. In some embodiments, A is:

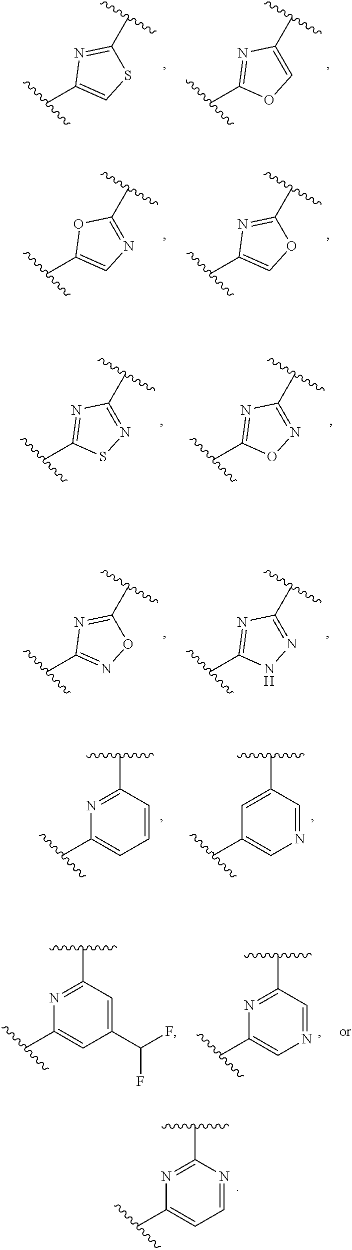

In some embodiments of a compound of the present invention, A is optionally substituted phenylene. In some embodiments, A is:

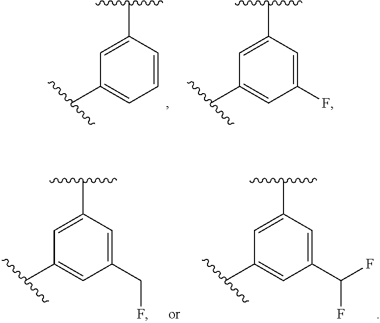 [SMILES: *c1cccc(*)c1 ; *c1cc(*)cc(F)c1 ; *c1cc(*)cc(CF)c1 ; *c1cc(*)cc(C(F)F)c1]

In some embodiments of a compound of the present invention, A is optionally substituted 3 to 6-membered heterocycloalkylene. In some embodiments, A is selected from the following, or a stereoisomer thereof:

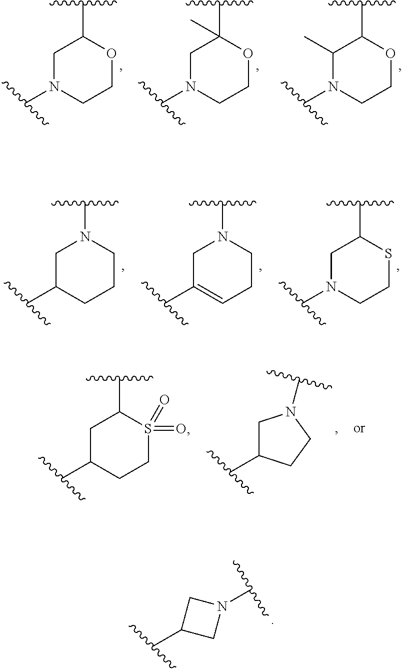

In some embodiments, A is selected from the following, or a stereoisomer thereof:

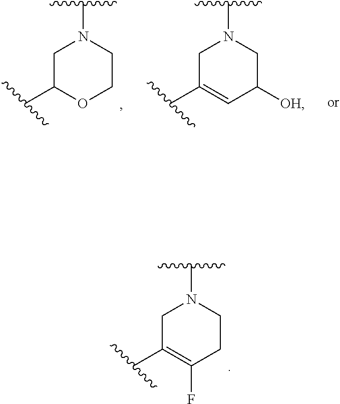 [SMILES: *C1CN(*)CCO1 ; *C1=CC(O)CN(*)C1 ; "or" ; *C1=C(F)CCN(*)C1]

In some embodiments of a compound of the present invention, the linker is the structure of Formula III:

A¹-(B¹)_{f}-(C¹)_{g}-(B²)ₕ-(D¹)-(B³)ᵢ—(C²)ⱼ-(B⁴)ₖ-A² Formula III,
wherein A¹ is a bond between the linker and CH(R³); A² is a bond between W and the linker; B¹, B², B³, and B⁴ each, independently, is selected from optionally substituted C₁-C₂ alkylene, optionally substituted C₁-C₃ heteroalkylene, O, S, and NR^{N}; each R^{N} is, independently, hydrogen, optionally substituted C₁-C₄ alkyl, optionally substituted C₂-C₄ alkenyl, optionally substituted C₂-C₄ alkynyl, optionally substituted 3 to 14-membered heterocycloalkyl, optionally substituted 6 to 10-membered aryl, or optionally substituted C₁-C₇ heteroalkyl; C¹ and C² are each, independently, selected from carbonyl, thiocarbonyl, sulphonyl, or phosphoryl; f, g, h, i, j, and k are each, independently, 0 or 1; and D¹ is optionally substituted C₁-C₁₀ alkylene, optionally substituted C₂-C₁₀ alkenylene, optionally substituted C₂-C₁₀ alkynylene, optionally substituted 3 to 14-membered heterocycloalkylene, optionally substituted 5 to 10-membered heteroarylene, optionally substituted 3 to 8-membered cycloalkylene, optionally substituted 6 to 10-membered arylene, optionally substituted C₂-C₁₀ polyethylene glycolene, or optionally substituted C₁-C₁₀ heteroalkylene, or a chemical bond linking A¹-(B¹)_{f}-(C¹)_{g}-(B²)ₕ- to -(B³)ᵢ-(C²)ⱼ-(B⁴)ₖ-A².

In some embodiments of a compound of the present invention, the linker is or comprises a cyclic moiety. In some embodiments, the linker has the structure of Formula IIIa:

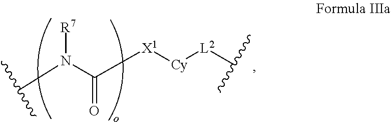

wherein o is 0 or 1;
R⁷ is hydrogen, optionally substituted C₁-C₆ alkyl, optionally substituted 3 to 8-membered cycloalkylene, or optionally substituted 3 to 8-membered heterocycloalkylene;
X¹ is absent, optionally substituted C₁-C₄ alkylene, O, NCH₃, or optionally substituted C₁-C₄ heteroalkylene;
Cy is optionally substituted 3 to 8-membered cycloalkylene, optionally substituted 3 to 12-membered heterocycloalkylene, optionally substituted 6-10 membered arylene, or optionally substituted 5 to 10-membered heteroarylene; and
L² is absent, —SO₂—, —NH—, optionally substituted C₁-C₄ alkylene, optionally substituted C₁-C₄ heteroalkylene, or optionally substituted 3 to 6-membered heterocycloalkylene.

In some embodiments, the linker is selected from, or a stereoisomer thereof:

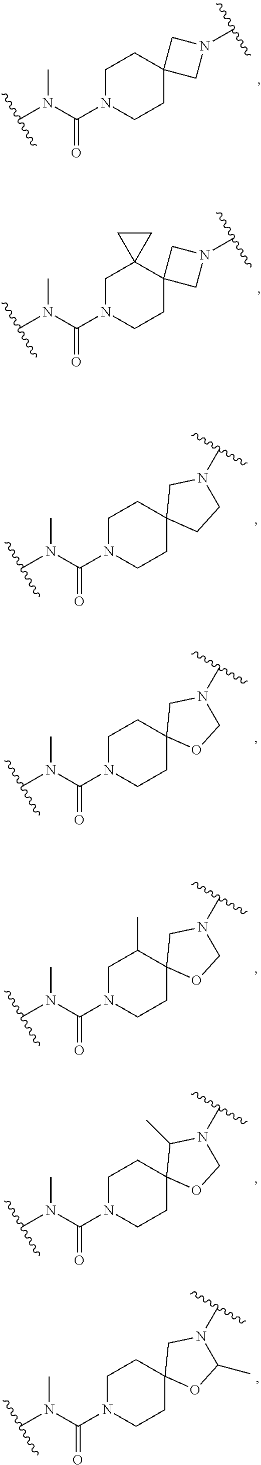

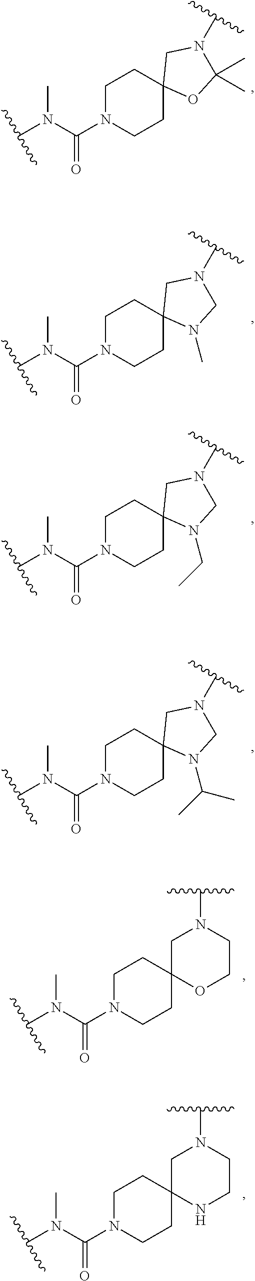 [SMILES: *N(C)C(=O)N1CCC2(CC1)CN(*)C(C)(C)O2 ; *N1CN(C)C2(CCN(C(=O)N(*)C)CC2)C1 ; *N1CN(CC)C2(CCN(C(=O)N(*)C)CC2)C1 ; *N1CN(C(C)C)C2(CCN(C(=O)N(*)C)CC2)C1 ; *N1CCOC2(CCN(C(=O)N(*)C)CC2)C1 ; *N1CCNC2(CCN(C(=O)N(*)C)CC2)C1]

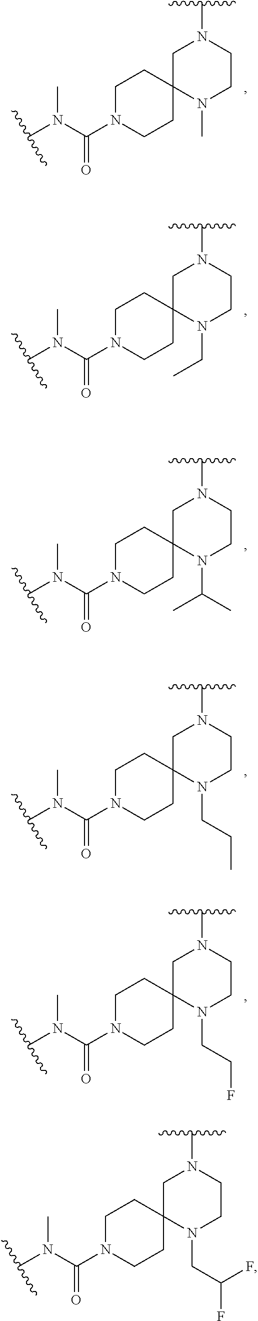 [SMILES: *N1CCN(C)C2(CCN(C(=O)N(*)C)CC2)C1 ; *N1CCN(CC)C2(CCN(C(=O)N(*)C)CC2)C1 ; *N1CCN(C(C)C)C2(CCN(C(=O)N(*)C)CC2)C1 ; *N1CCN(CCC)C2(CCN(C(=O)N(*)C)CC2)C1 ; *N1CCN(CCF)C2(CCN(C(=O)N(*)C)CC2)C1 ; *N1CCN(CC(F)F)C2(CCN(C(=O)N(*)C)CC2)C1]

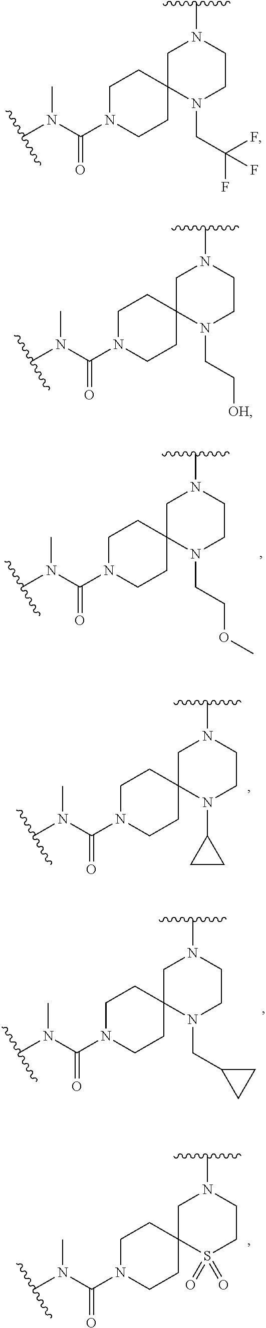

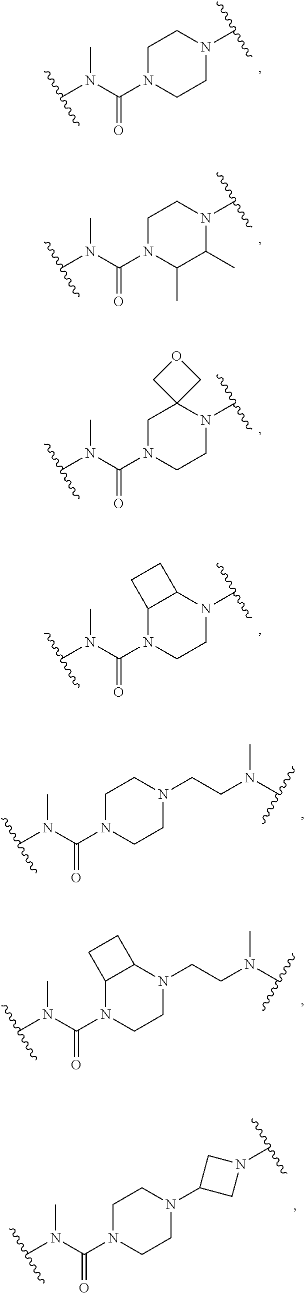

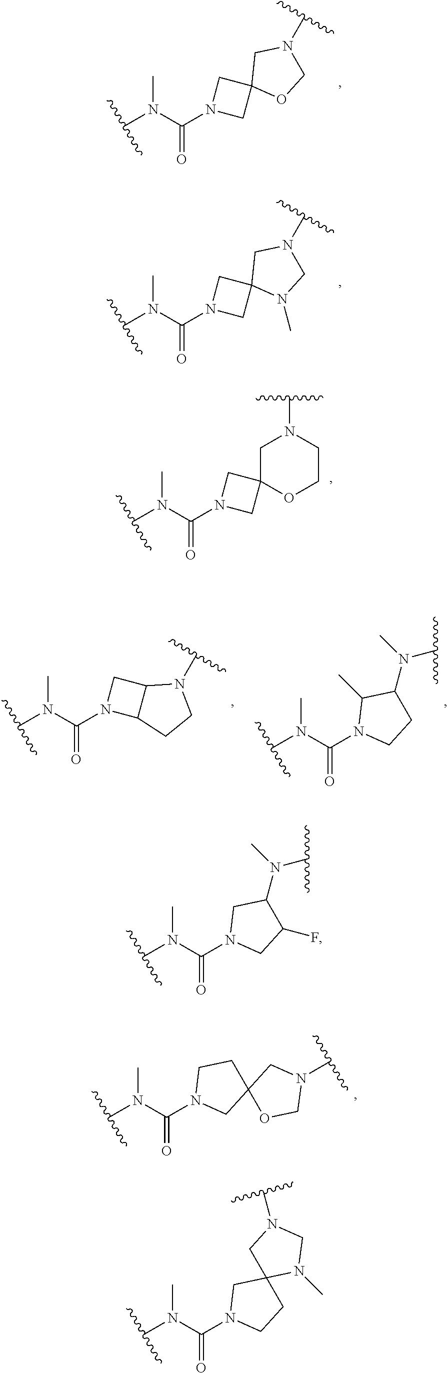

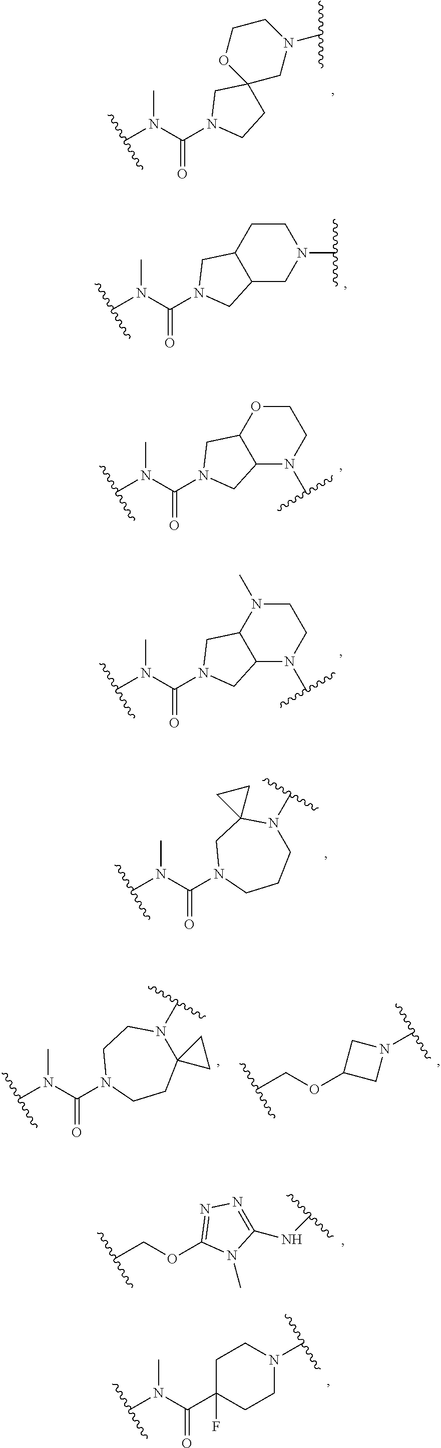

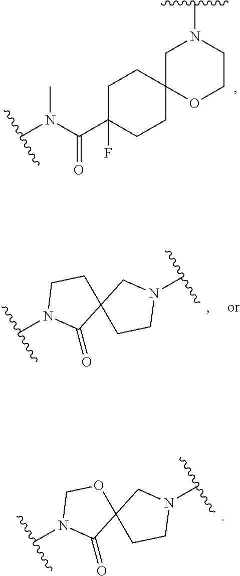 [SMILES: *N1CCOC2(CCC(F)(C(=O)N(*)C)CC2)C1 ; *N1CCC2(CCN(*)C2=O)C1 ; *N1CCC2(C1)OCN(*)C2=O]

In some embodiments, the linker is selected from, or a stereoisomer thereof:

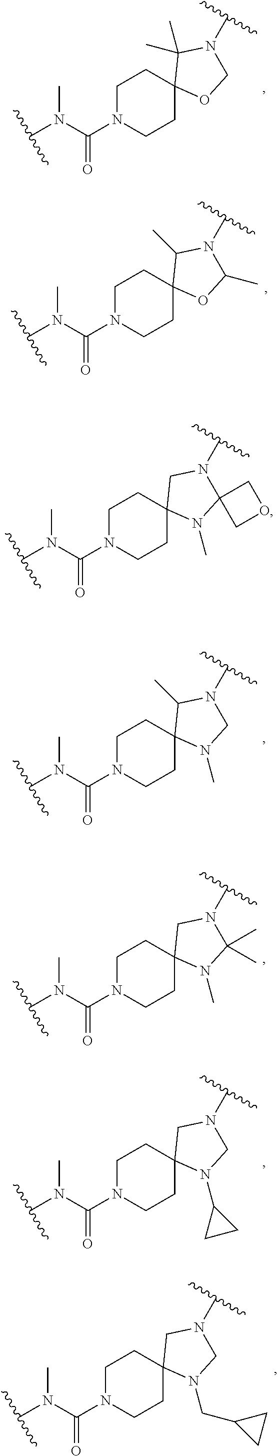

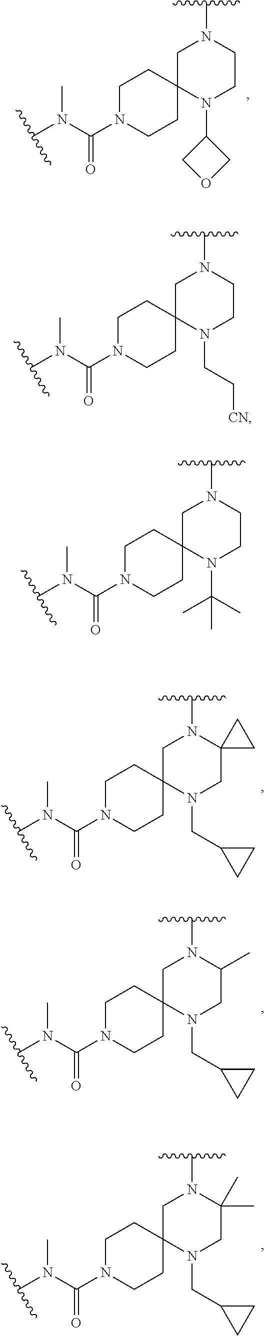 [SMILES: *N1CCN(C2COC2)C2(CCN(C(=O)N(*)C)CC2)C1 ; *N1CCN(CCC#N)C2(CCN(C(=O)N(*)C)CC2)C1 ; *N1CCN(C(C)(C)C)C2(CCN(C(=O)N(*)C)CC2)C1 ; *N(C)C(=O)N1CCC2(CC1)CN(*)C1(CC1)CN2CC1CC1 ; *N(C)C(=O)N1CCC2(CC1)CN(*)C(C)CN2CC1CC1 ; *N(C)C(=O)N1CCC2(CC1)CN(*)C(C)(C)CN2CC1CC1]

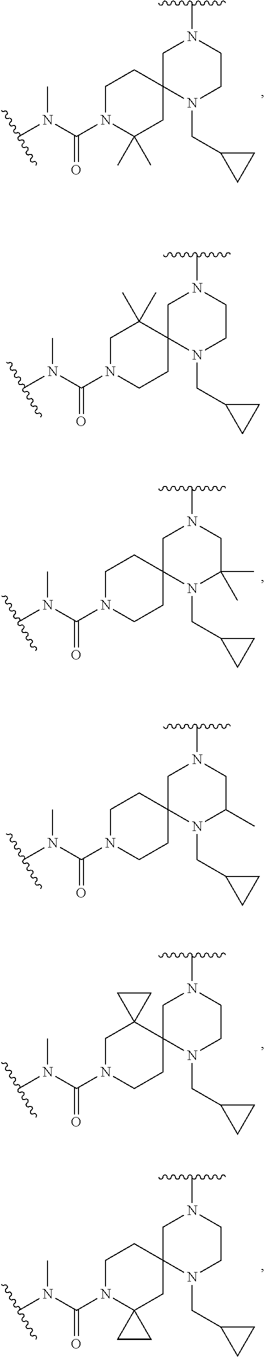 [SMILES: *N1CCN(CC2CC2)C2(CCN(C(=O)N(*)C)C(C)(C)C2)C1 ; *N1CCN(CC2CC2)C2(CCN(C(=O)N(*)C)CC2(C)C)C1 ; *N1CC(C)(C)N(CC2CC2)C2(CCN(C(=O)N(*)C)CC2)C1 ; *N1CC(C)N(CC2CC2)C2(CCN(C(=O)N(*)C)CC2)C1 ; *N1CCN(CC2CC2)C2(CCN(C(=O)N(*)C)CC23CC3)C1 ; *N1CCN(CC2CC2)C2(CCN(C(=O)N(*)C)C3(CC3)C2)C1]

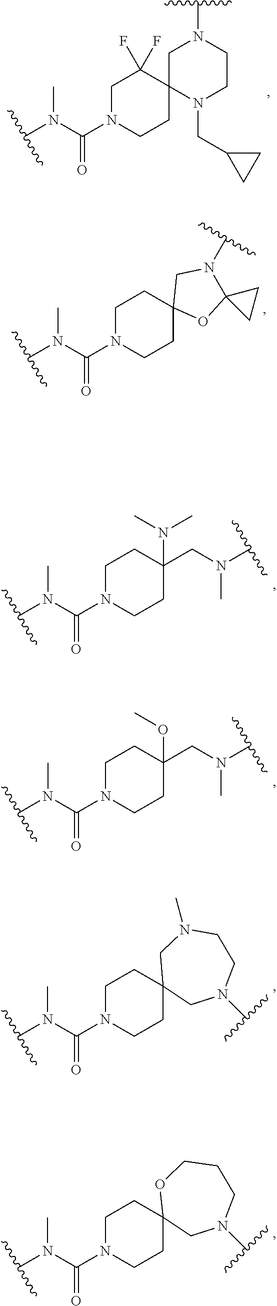 [SMILES: *N1CCN(CC2CC2)C2(CCN(C(=O)N(*)C)CC2(F)F)C1 ; *N(C)C(=O)N1CCC2(CC1)CN(*)C1(CC1)O2 ; *N(C)CC1(N(C)C)CCN(C(=O)N(*)C)CC1 ; *N(C)CC1(OC)CCN(C(=O)N(*)C)CC1 ; *N1CCN(C)CC2(CCN(C(=O)N(*)C)CC2)C1 ; *N1CCCOC2(CCN(C(=O)N(*)C)CC2)C1]

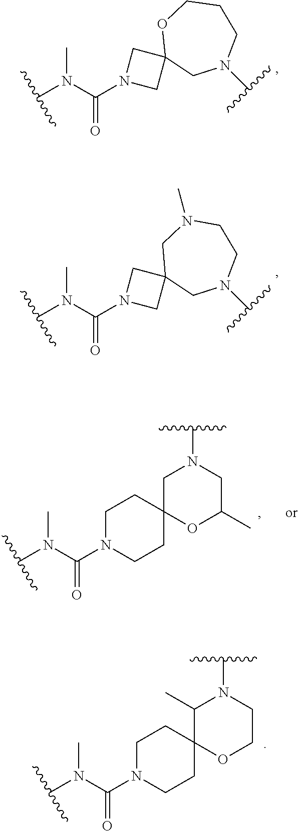 [SMILES: *N1CCCOC2(C1)CN(C(=O)N(*)C)C2 ; *N1CCN(C)CC2(C1)CN(C(=O)N(*)C)C2 ; *N1CC(C)OC2(CCN(C(=O)N(*)C)CC2)C1 ; *N(C)C(=O)N1CCC2(CC1)OCCN(*)C2C]

In some embodiments, a compound of the present invention has the structure of Formula II-5, or a pharmaceutically acceptable salt thereof:

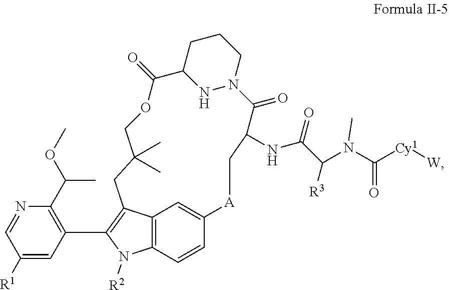

wherein Cy¹ is optionally substituted spirocyclic 8 to 11-membered heterocycloalkylene or optionally substituted bicyclic 7 to 9-membered heterocycloalkylene; and
wherein W comprises a vinyl ketone or a vinyl sulfone.

In some embodiments, Cy¹ is optionally substituted spirocyclic 10 to 11-membered heterocycloalkylene.

In some embodiments, a compound of the present invention has the structure of Formula II-5a:

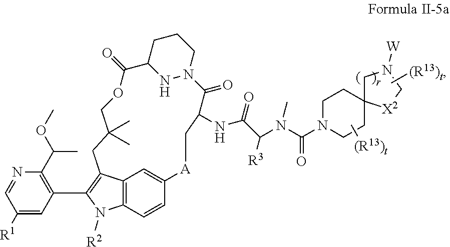

wherein X² is O, C(R¹¹)₂, NR¹², S, or SO₂.
r is 1 or 2;
each t is, independently, 0, 1, or 2;
R¹¹ and R¹² are each, independently, hydrogen, optionally substituted C₁-C₄ alkyl, optionally substituted C₂-C₄ heteroalkyl, or optionally substituted 3 to 5-membered cycloalkyl; and
each R¹³ is, independently, —CH₃.

In some embodiments, a compound of the present invention has the structure of Formula II-5b:

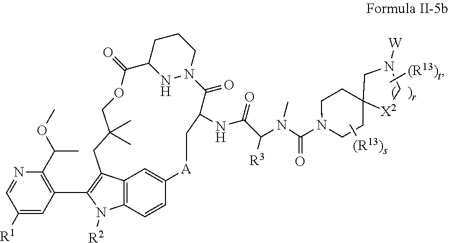

wherein X² is O, C(R¹¹)₂, NR¹², S, or SO₂.
r is 1 or 2;
s and t are each, independently, 0, 1, or 2;
R¹¹ and R¹² are each, independently, hydrogen, optionally substituted C₁-C₄ alkyl, optionally substituted C₂-C₄ heteroalkyl, optionally substituted 3- to 6-membered heterocycloalkyl, or optionally substituted 3 to 5-membered cycloalkyl; and
each R¹³ is, independently, —CH₃, F, or two R¹³ attached to the same atom combine with the atom to which they are attached to form an optionally substituted C₃-C₆ cycloalkyl, or two R¹³ attached to the same atom combine with the atom to which they are attached to form an optionally substituted 3- to 6-membered heterocycloalkyl.

In some embodiments, R¹³ is —CH₃.

In some embodiments, the sum of s and t is 1. In some embodiments, the sum of s and t is 2. In some embodiments, s is 0 and tis 1. In some embodiments, the sum of s and tis 0

In some embodiments, a compound of the present invention has the structure of Formula II-5c:

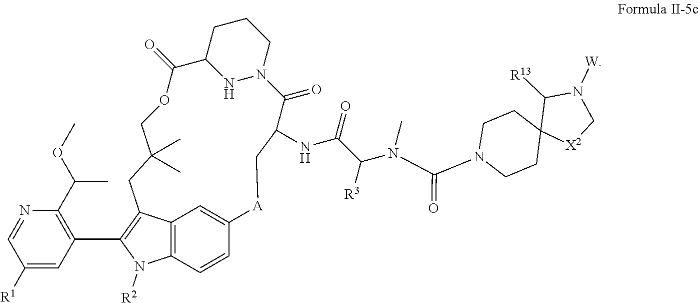

In some embodiments, a compound of the present invention has the structure of Formula II-5d:

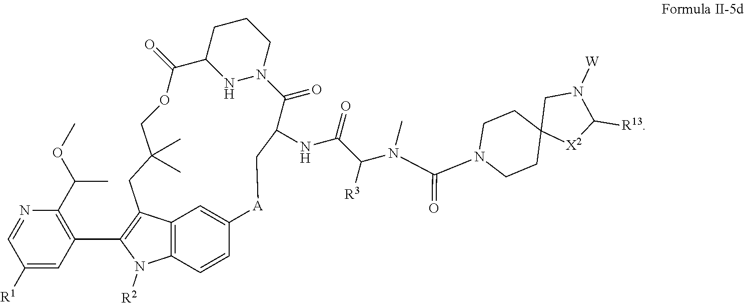

In some embodiments, a compound of the present invention has the structure of Formula II-5e:

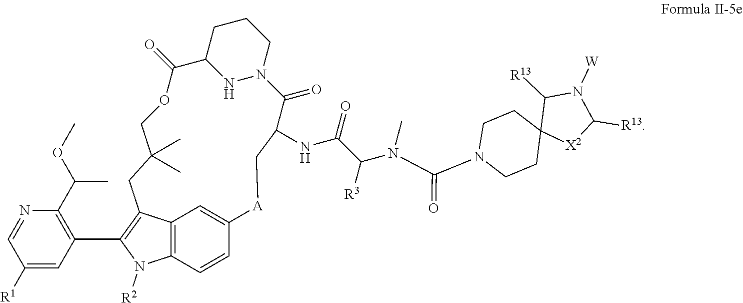

In some embodiments, r is 1. In some embodiments, r is 2. In some embodiments, X² is O. In some embodiments, X² is S. In some embodiments, X² is SO₂.

In some embodiments, X² is NR¹². In some embodiments, R¹² is selected from, or a stereoisomer thereof:

### —CH₃.

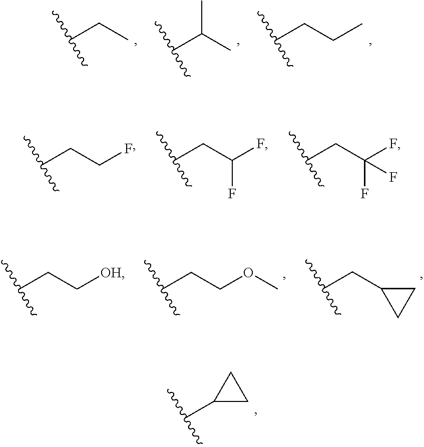

or —H. In some embodiments, R¹² is selected from, or a stereoisomer thereof:

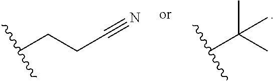

In some embodiments, X² is C(R¹¹)₂. In some embodiments, each R¹¹ is hydrogen.

In some embodiments of a compound of the present invention, W is a cross-linking group comprising a vinyl ketone. In some embodiments, W has the structure of Formula IVa:

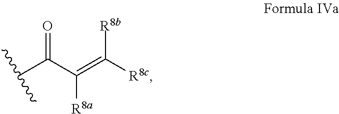

wherein R^{8a}, R^{8b}, and R^{8c} are, independently, hydrogen, —CN, halogen, or —C₁-C₃ alkyl optionally substituted with one or more substituents independently selected from —OH, —O—C₁-C₃ alkyl, —NH₂, —NH(C₁-C₃ alkyl), —N(C₁-C₃ alkyl)₂, or a 4 to 7-membered saturated heterocycloalkyl. In some embodiments, W is selected from, or a stereoisomer thereof:

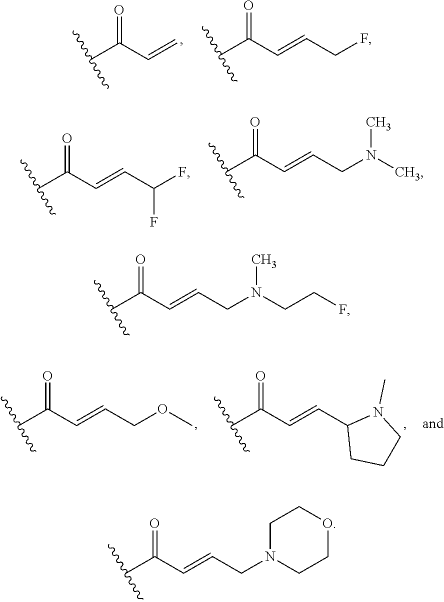

In some embodiments, W is selected from, or a stereoisomer thereof:

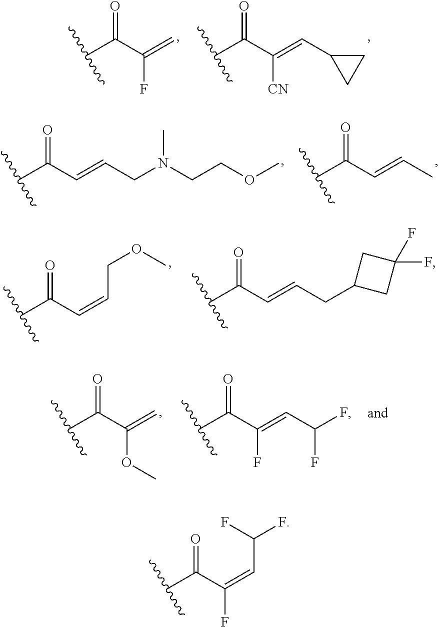

In some embodiments of a compound of the present invention, W is a cross-linking group comprising a vinyl sulfone. In some embodiments, W has the structure of Formula IVc:

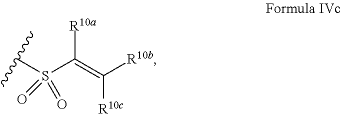

wherein R^{10a}, R^{10b}, and R^{10c} are, independently, hydrogen, —CN, or —C₁-C₃ alkyl optionally substituted with one or more substituents independently selected from —OH, —O—C₁-C₃ alkyl, —NH₂, —NH(C₁-C₃ alkyl), —N(C₁-C₈alkyl)₂, or a 4 to 7-membered saturated heterocycloalkyl. In some embodiments, W is:

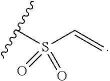 [SMILES: *S(=O)(=O)C=C]

In some embodiments of a compound of the present invention, W is a cross-linking group comprising an ynone. In some embodiments, W has the structure of Formula IVb:

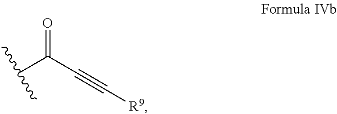

wherein R⁹ is hydrogen, —C₁-C₃ alkyl optionally substituted with one or more substituents independently selected from —OH, —O—C₁-C₃ alkyl, —NH₂, —NH(C₁-C₃ alkyl), —N(C₁-C₃ alkyl)₂, or a 4 to 7-membered saturated cycloalkyl, or a 4 to 7-membered saturated heterocycloalkyl. In some embodiments, W is selected from:

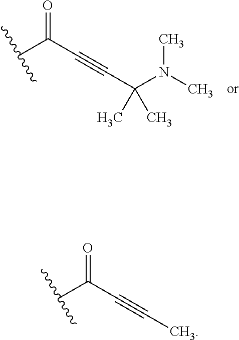 [SMILES: *C(=O)C#CC(C)(C)N(C)C ; *C(=O)C#CC]

In some embodiments, a compound of the present invention has the structure of Formula II-6:

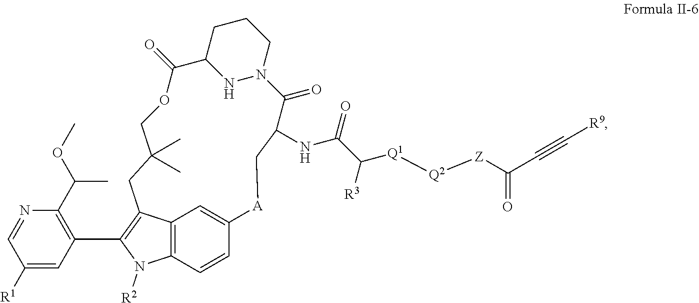

wherein Q¹ is CH₂, NR^{N}, or O;
Q² is CO, NR^{N}, or O; and
Z is optionally substituted 3 to 6-membered heterocycloalkylene or optionally substituted 5 to 10-membered heteroarylene; or
wherein Q¹-Q²-Z is an optionally substituted 9 to 10-membered spirocyclic heterocycloalkylene.

In some embodiments, a compound of the present invention has the structure of Formula II-6a:

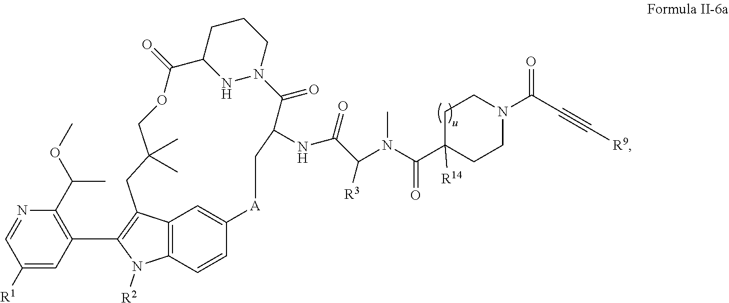

wherein R¹⁴ is fluoro, hydrogen, or C₁-C₃ alkyl; and
u is 0 or 1.

In some embodiments, R¹⁴ is fluoro and u is 1. In some embodiments, R¹⁴ is hydrogen and u is 0.

In some embodiments, a compound of the present invention has the structure of Formula II-6b:

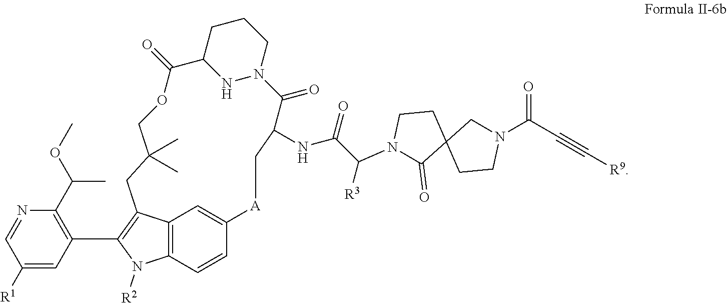

In some embodiments, a compound of the present invention has the structure of Formula II-6c:

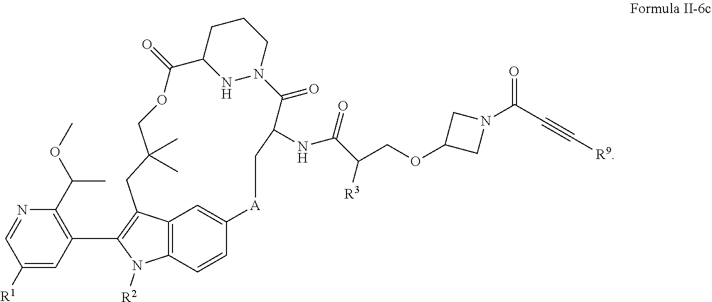

In some embodiments, a compound of the present invention is selected from Table 1, or a pharmaceutically acceptable salt or stereoisomer thereof. In some embodiments, a compound of the present invention is selected from Table 1, or a pharmaceutically acceptable salt or atropisomer thereof. In some embodiments, the compound is a compound selected from A1 to A209 of Table 1. In some embodiments, the compound is a compound selected from A210 to A368 of Table 1.

TABLE 1
Certain Compounds of the Present Invention

| | Invention |
|---|---|
| Ex # | Structure |
| A1 | 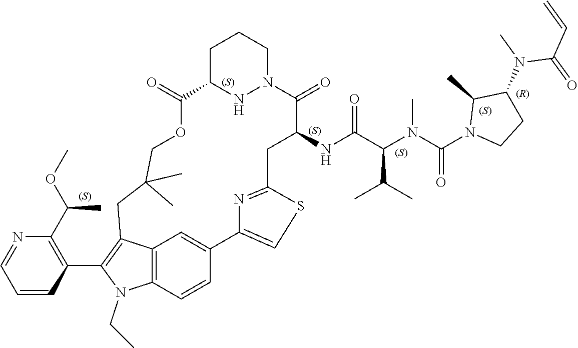 |
| A2 | 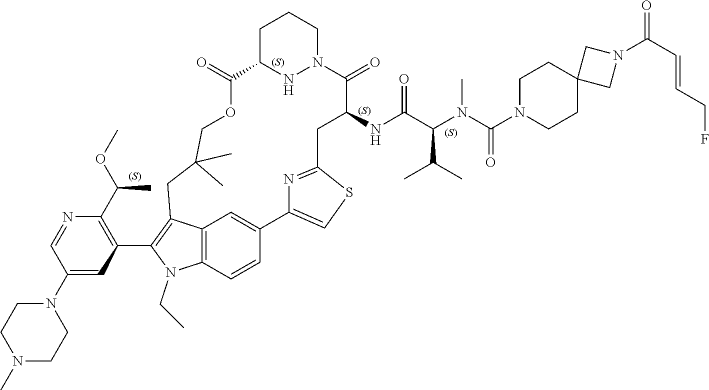 |
| A3 | 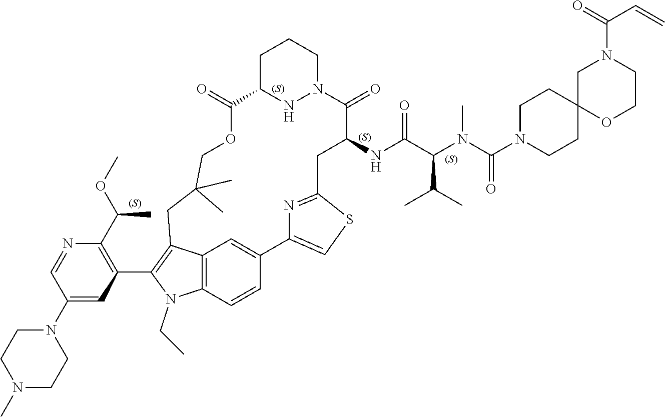 |
| A4 | 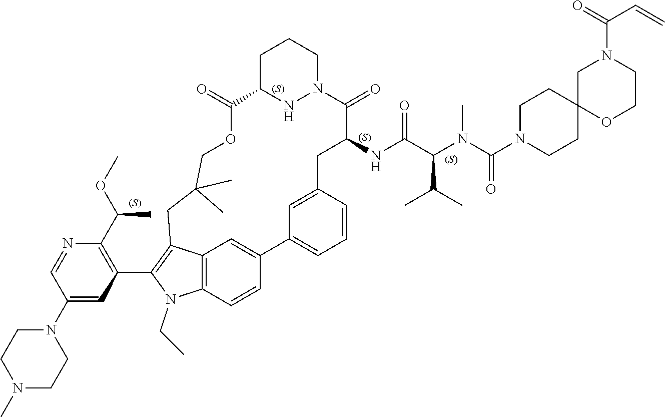 |
| A5 | 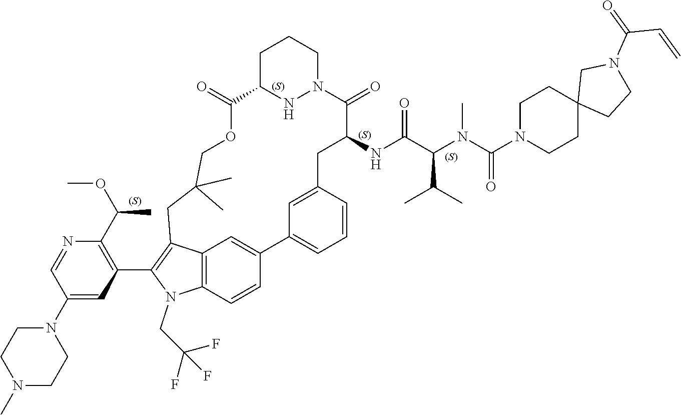 |
| A6 | 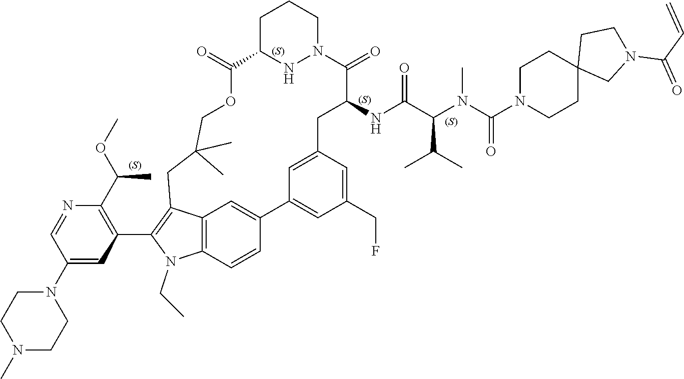 |
| A7 | 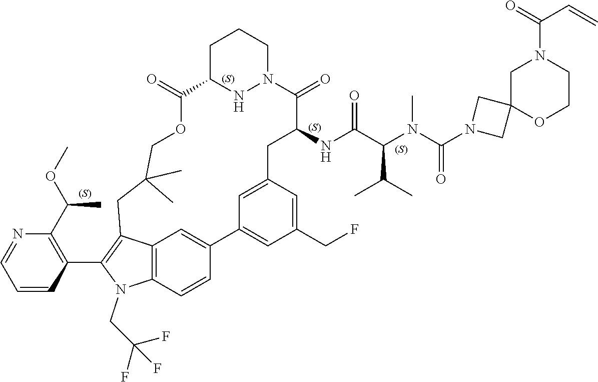 |
| A8 | 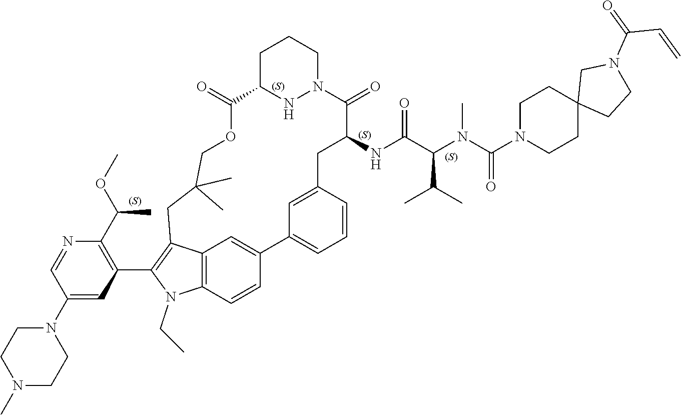 |
| A9 | 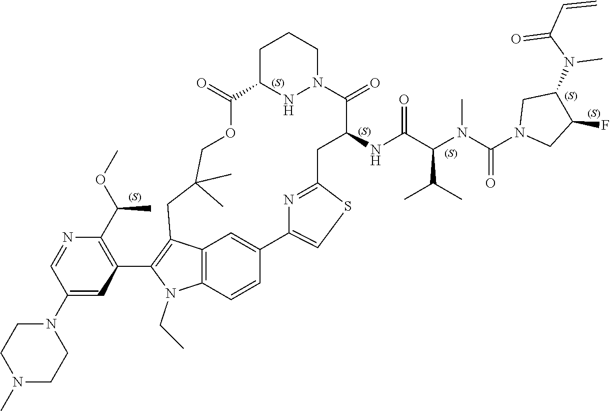 |
| A10 | 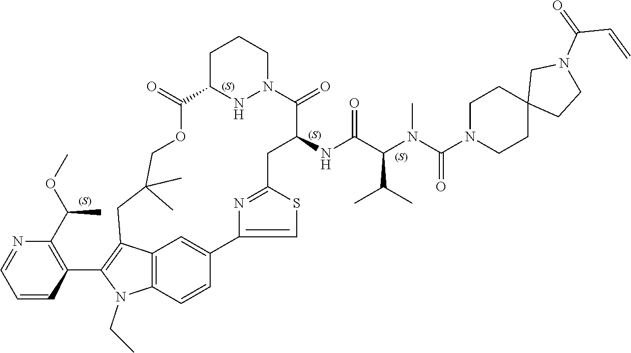 |
| A11 | 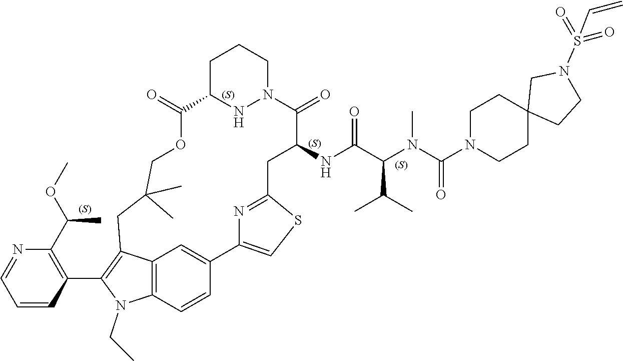 |
| A12 | 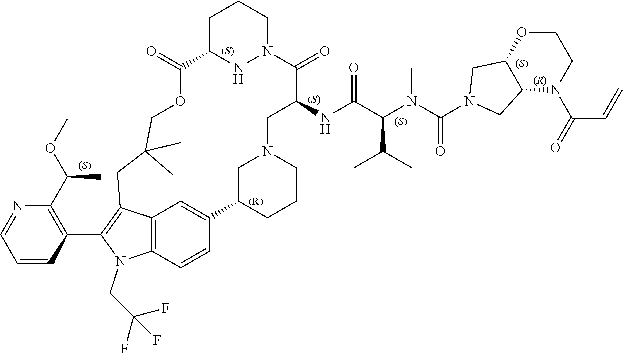 |
| A13 | 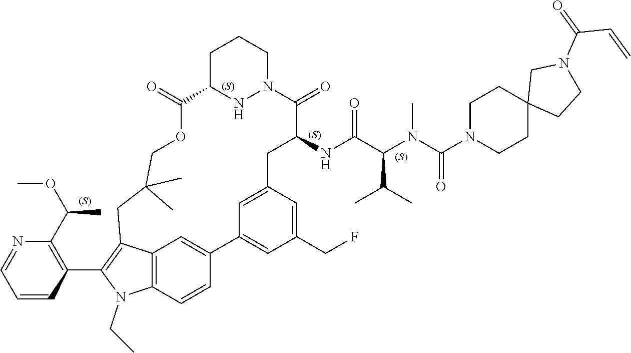 |
| A14 | 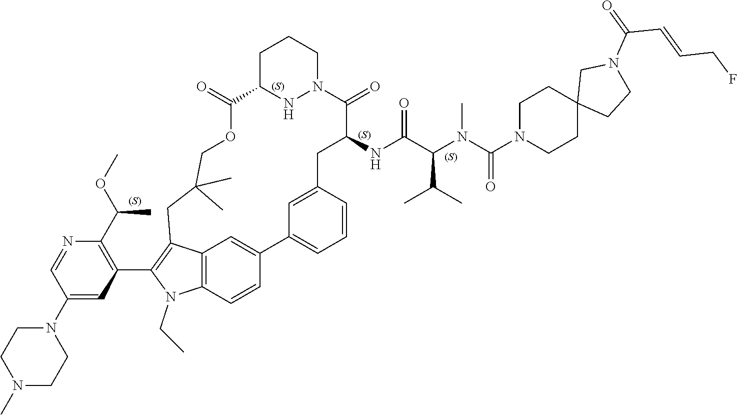 |
| A15 | 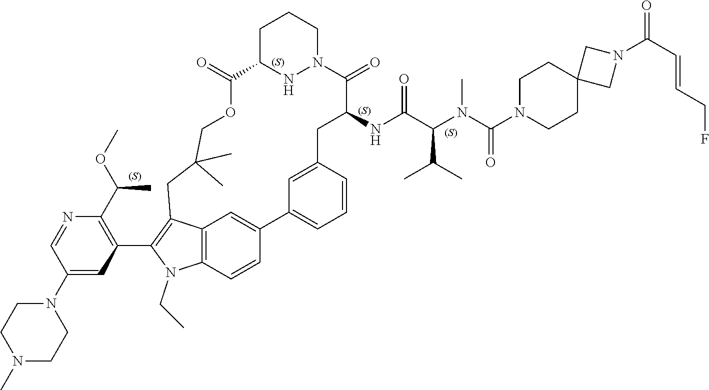 |
| A16 | 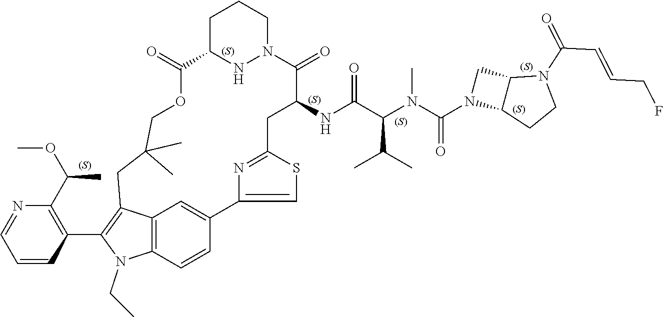 |
| A17 | 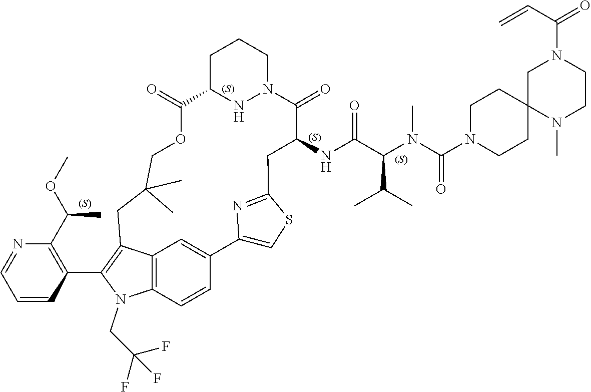 |
| A18 | 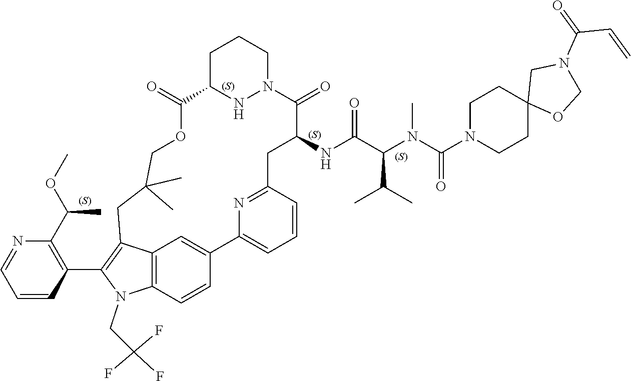 |
| A19 | 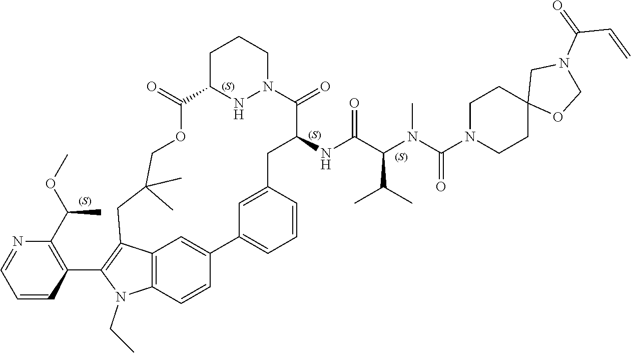 |
| A20 | 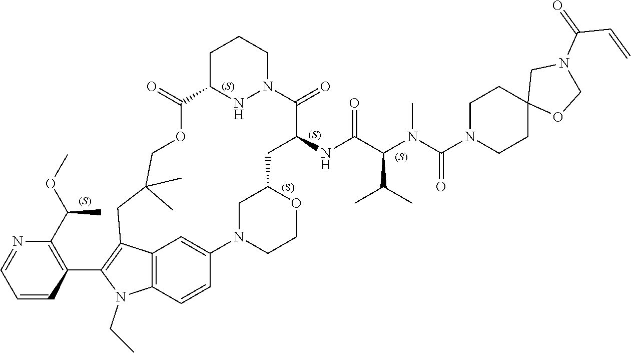 |
| A21 | 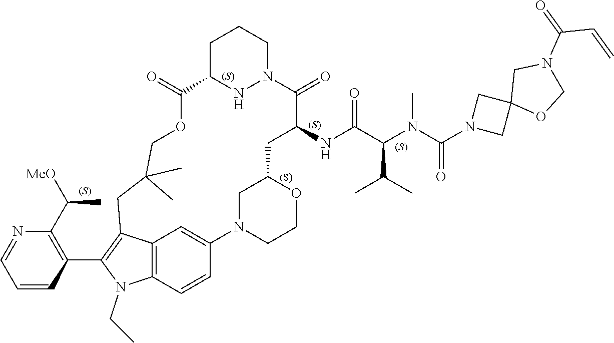 |
| A22 | 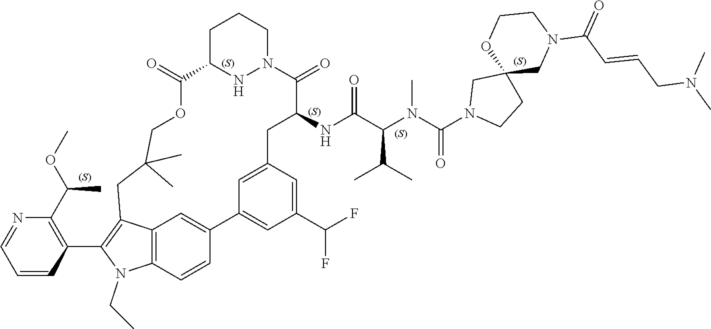 |
| A23 | 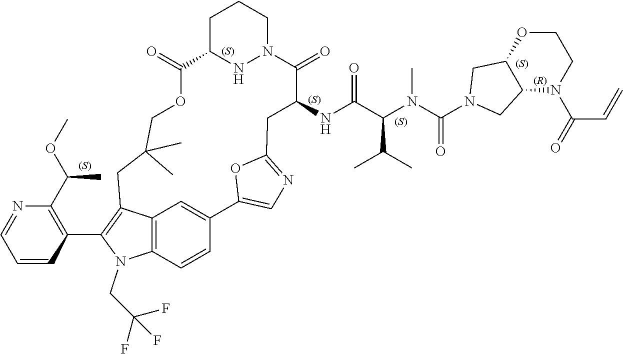 |
| A24 | 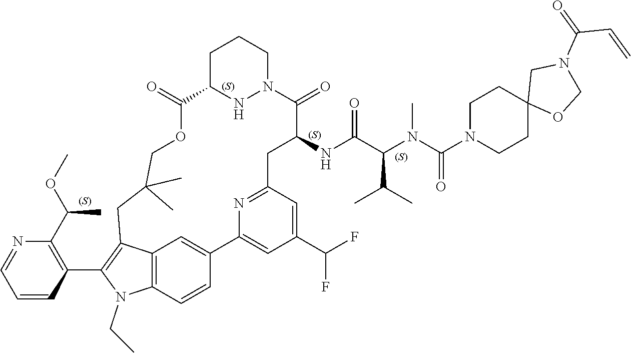 |
| A25 | 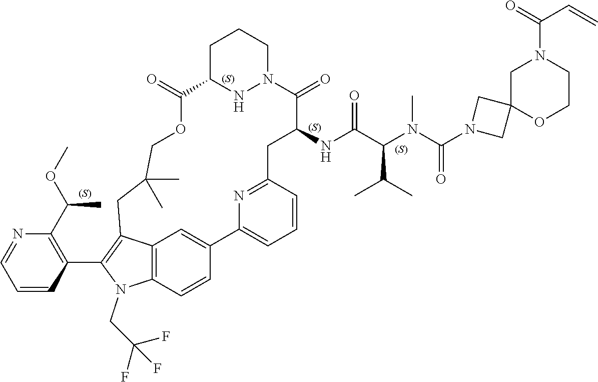 |
| A26 | 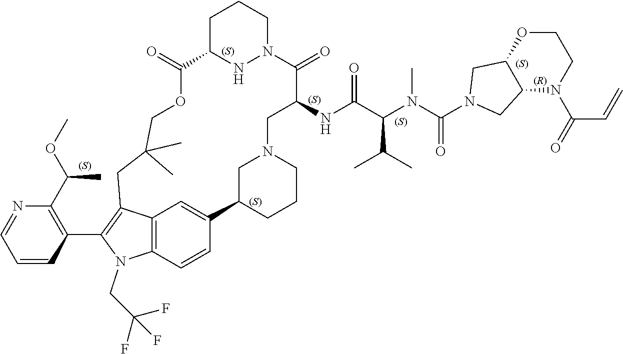 |
| A27 | 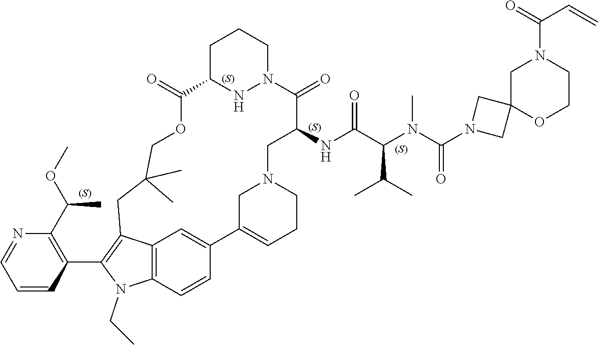 |
| A28 | 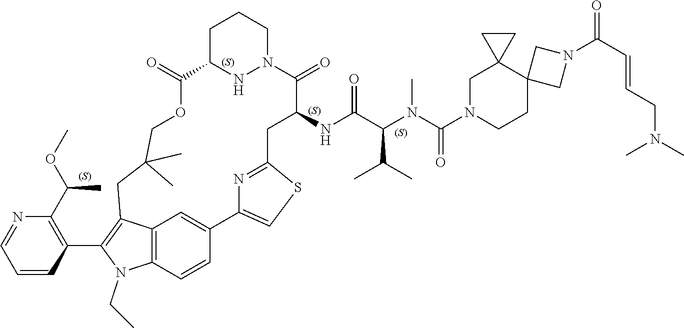 |
| A29 | 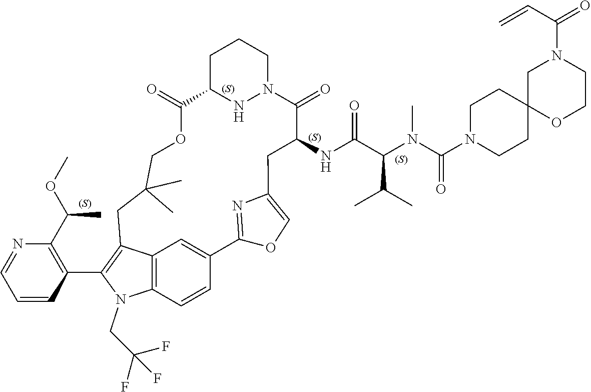 |
| A30 | 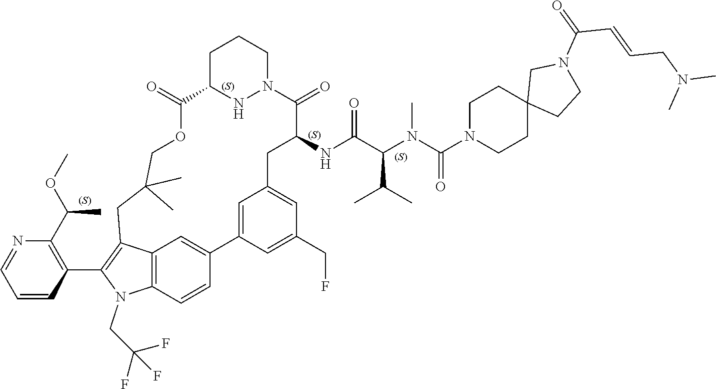 |
| A31 | 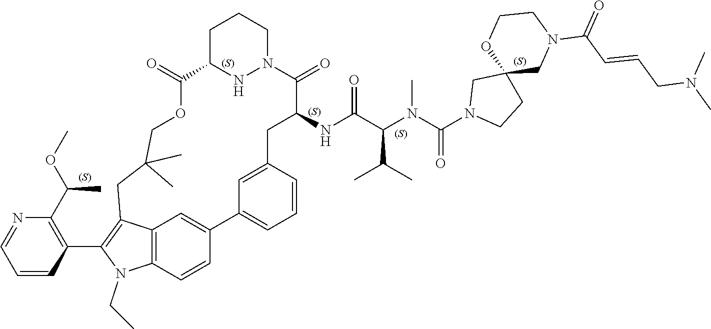 |
| A32 | 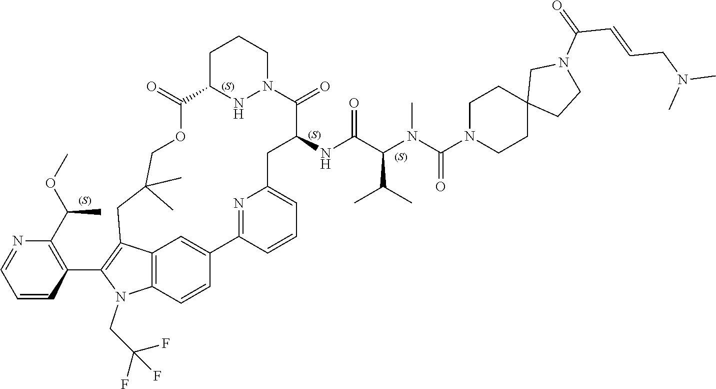 |
| A33 | 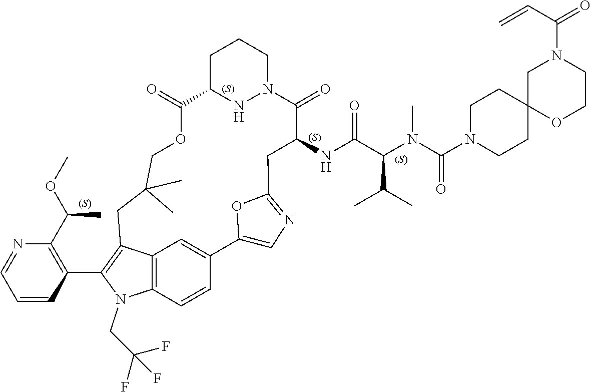 |
| A34 | 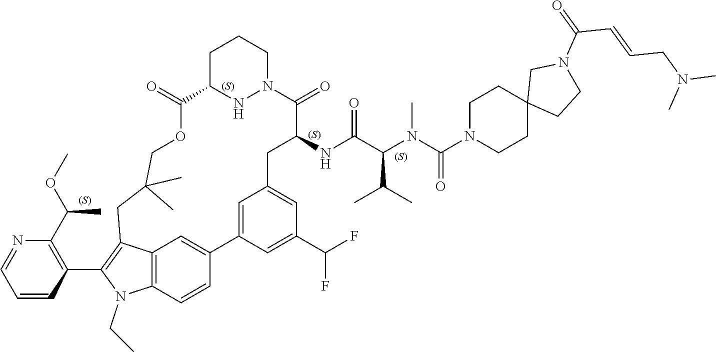 |
| A35 | 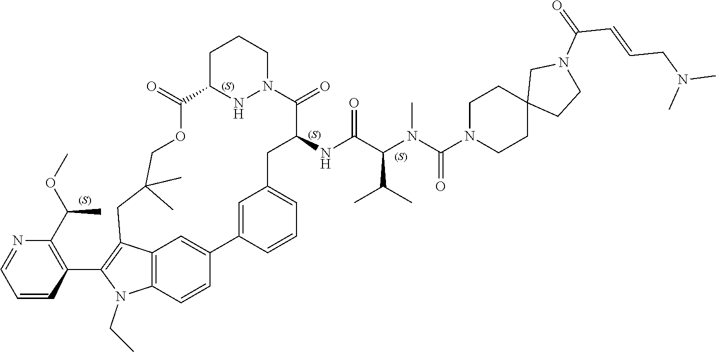 |
| A36 | 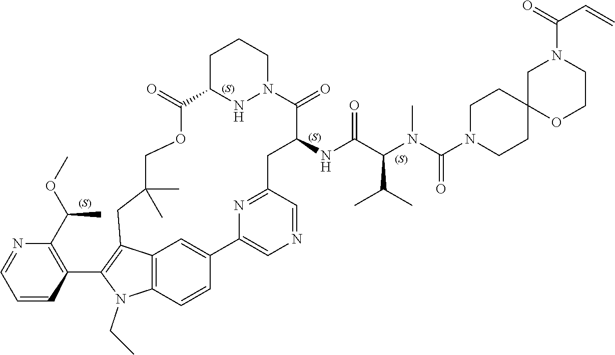 |
| A37 | 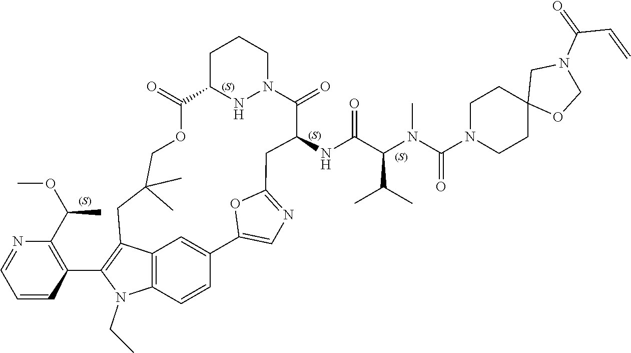 |
| A38 | 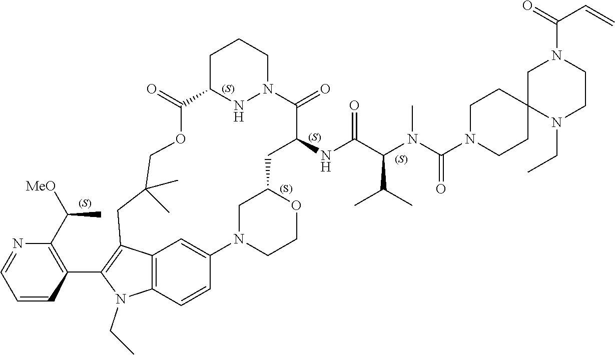 |
| A39 | 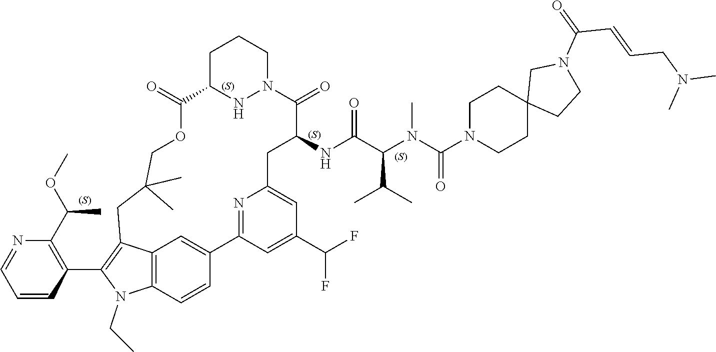 |
| A40 | 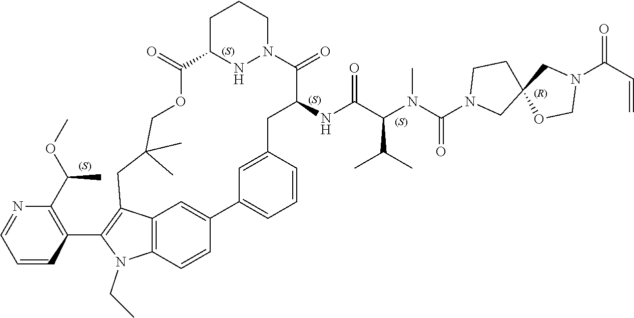 |
| A41 | 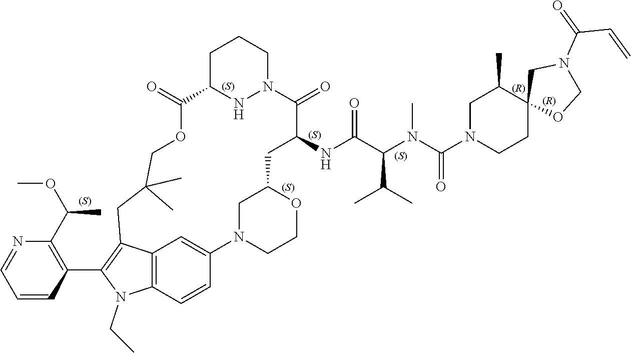 |
| A42 | 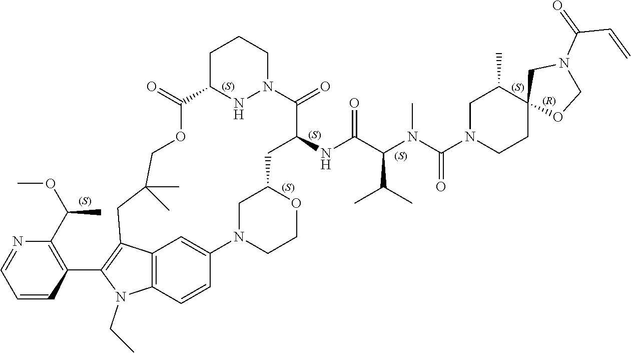 |
| A43 | 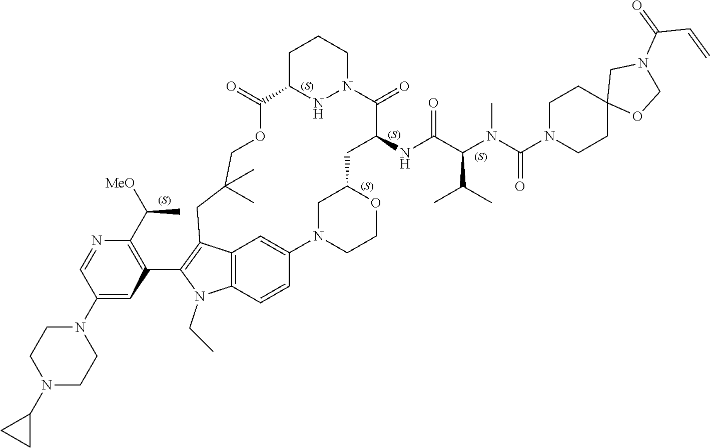 |
| A44 | 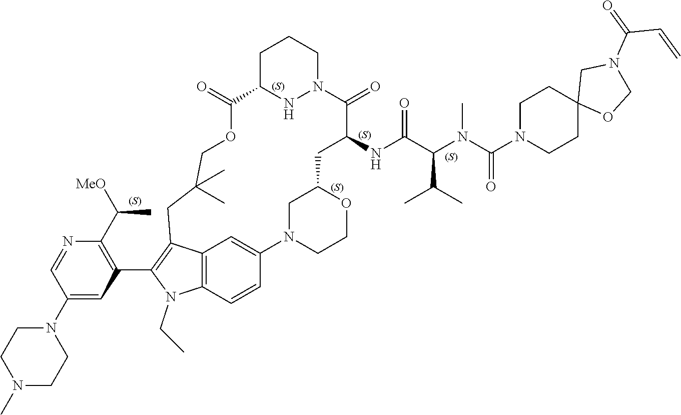 |
| A45 | 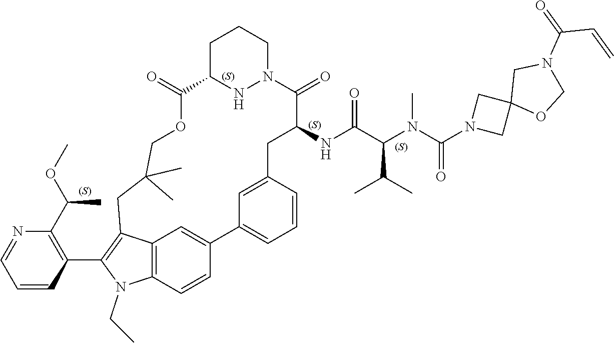 |
| A46 | 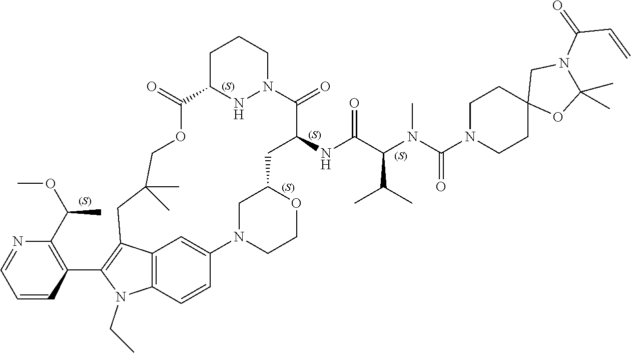 |
| A47 | 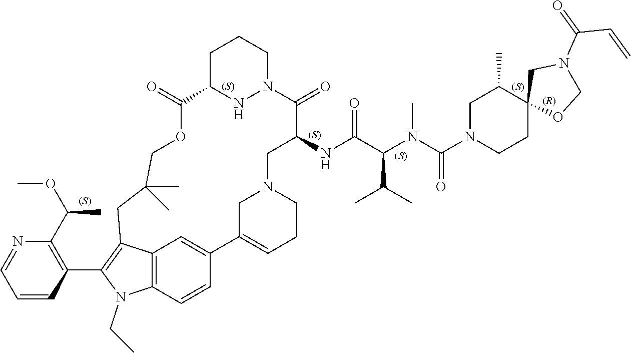 |
| A48 | 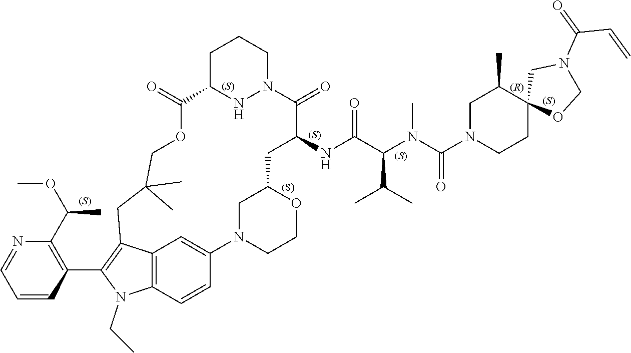 |
| A49 | 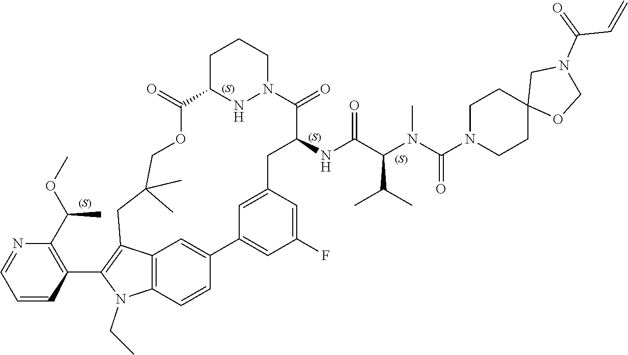 |
| A50 | 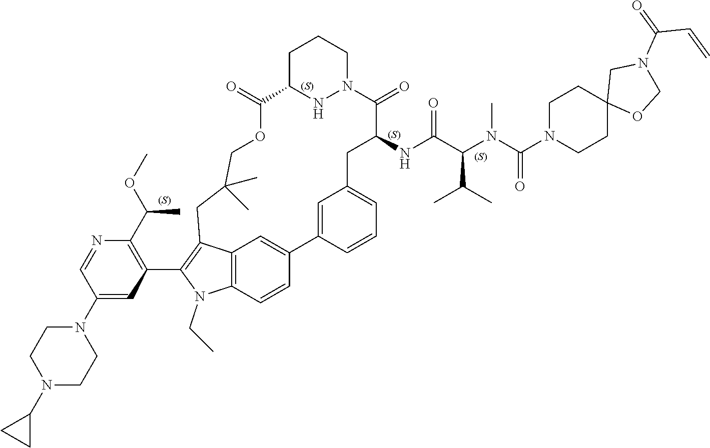 |
| A51 | 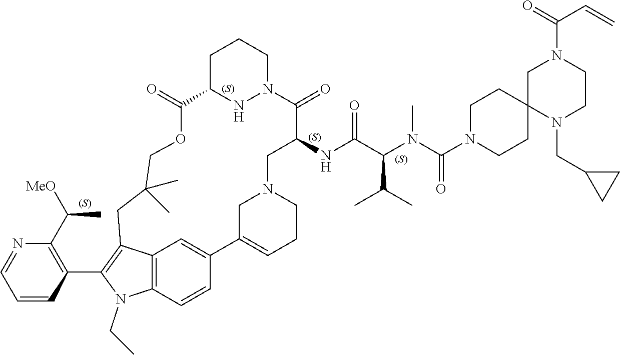 |
| A52 | 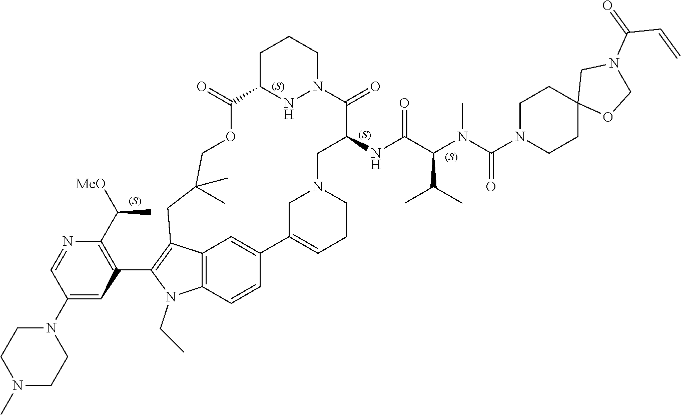 |
| A53 | 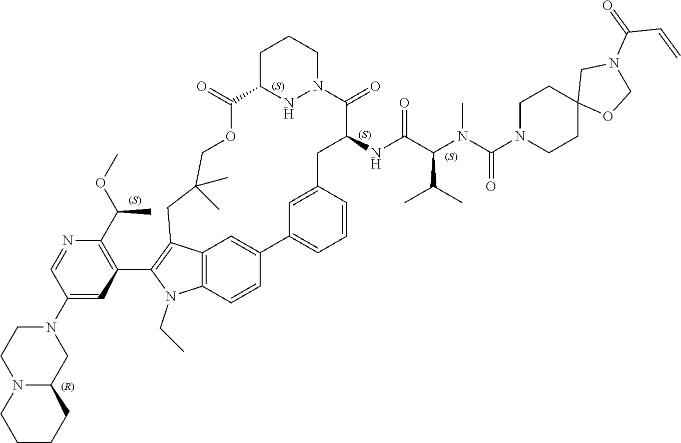 |
| A54 | 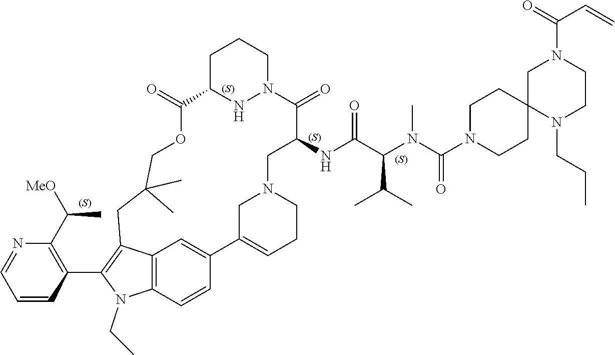 |
| A55 | 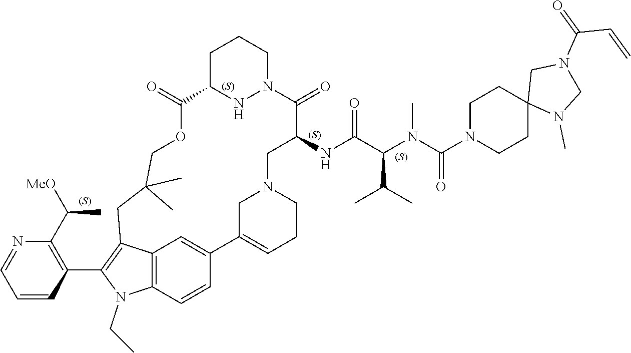 |
| A56 | 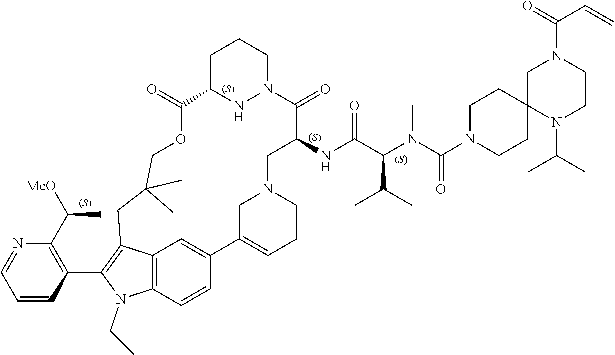 |
| A57 | 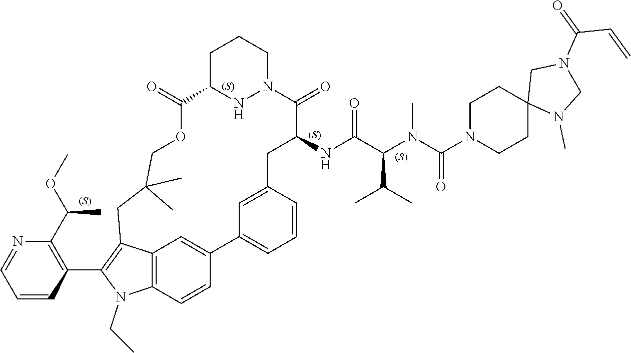 |
| A58 | 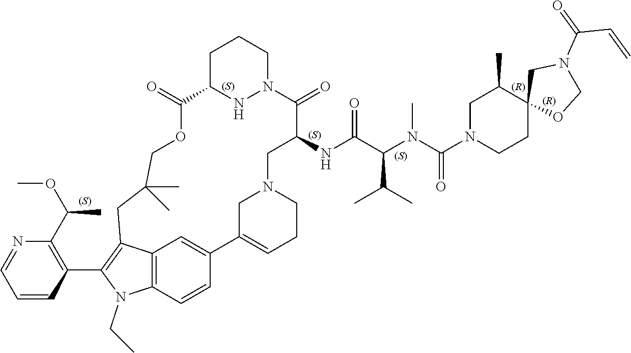 |
| A59 | 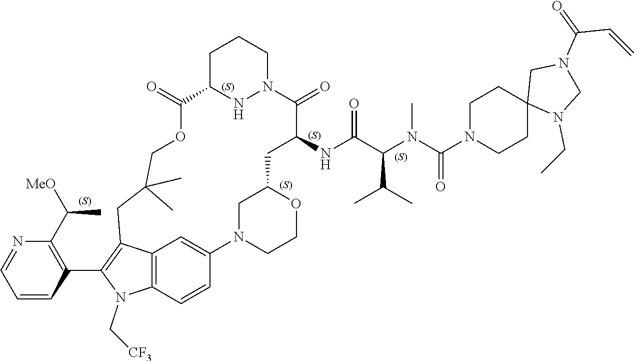 |
| A60 | 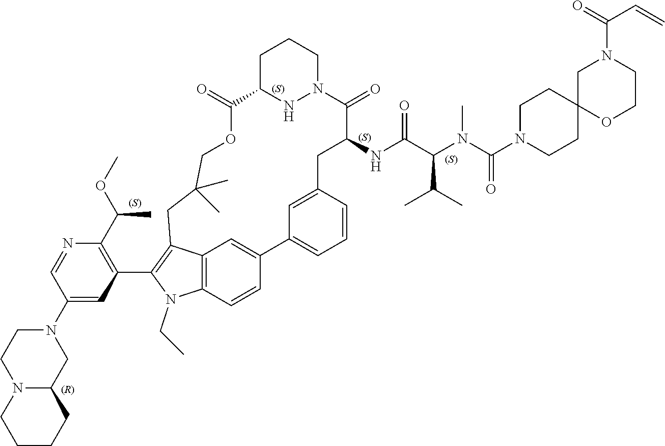 |
| A61 | 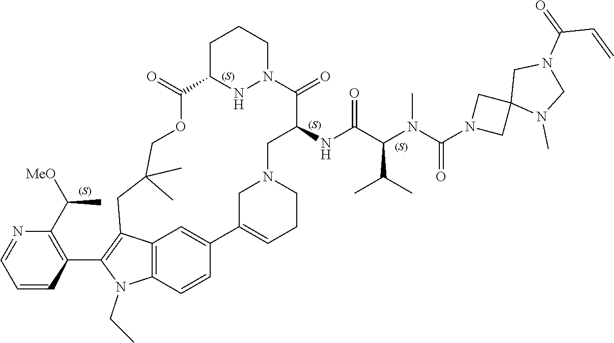 |
| A62 | 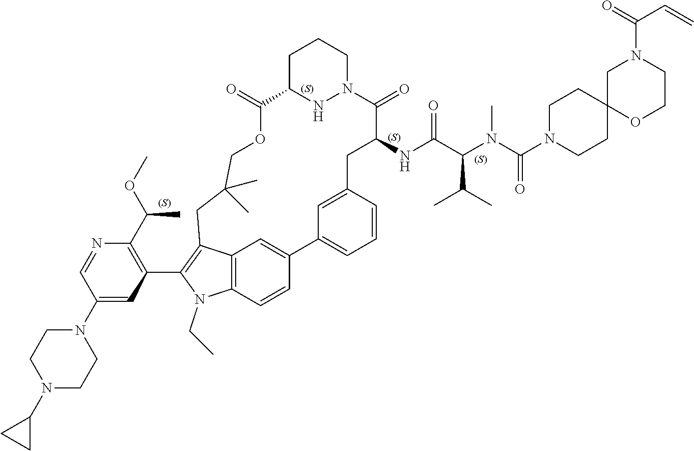 |
| A63 | 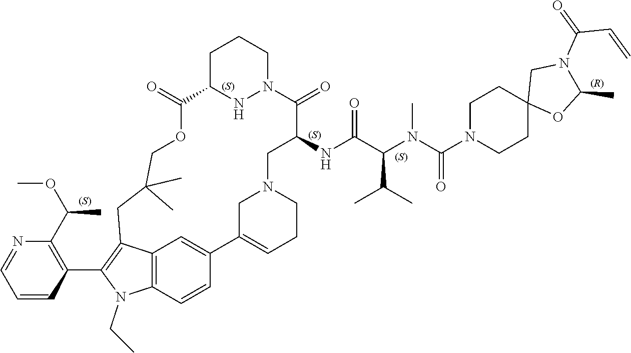 |
| A64 | 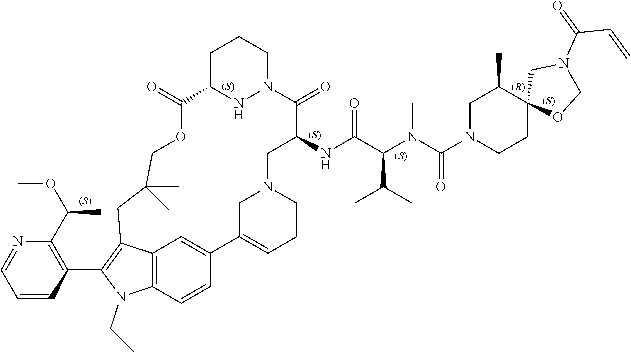 |
| A65 | 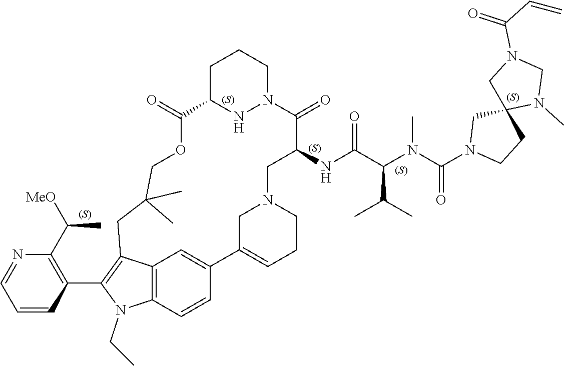 |
| A66 | 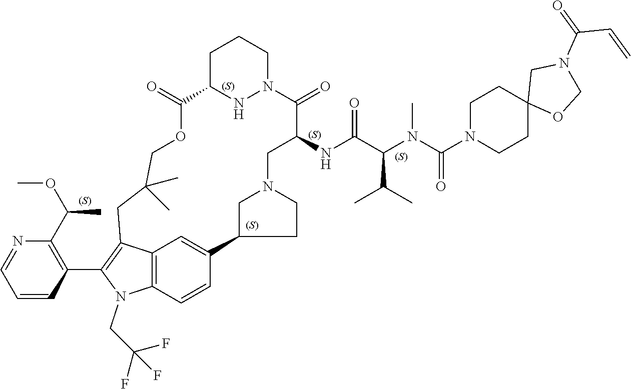 |
| A67 | 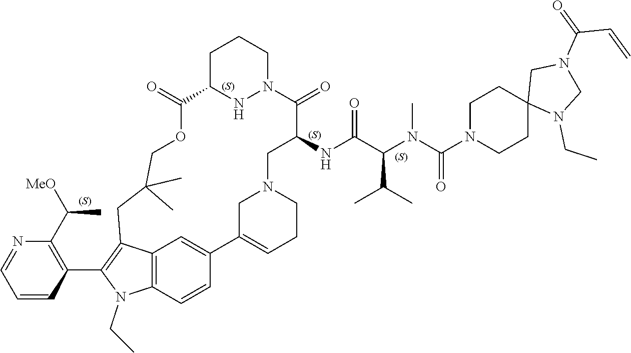 |
| A68 | 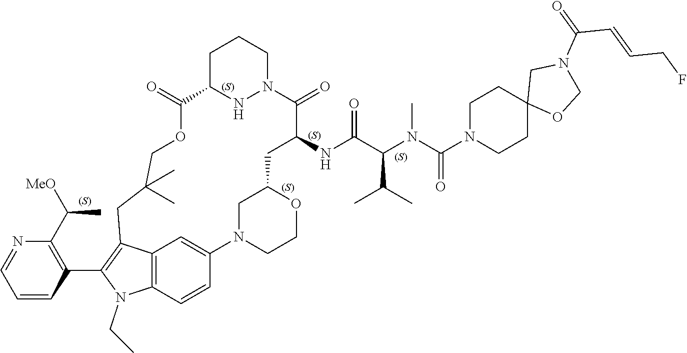 |
| A69 | 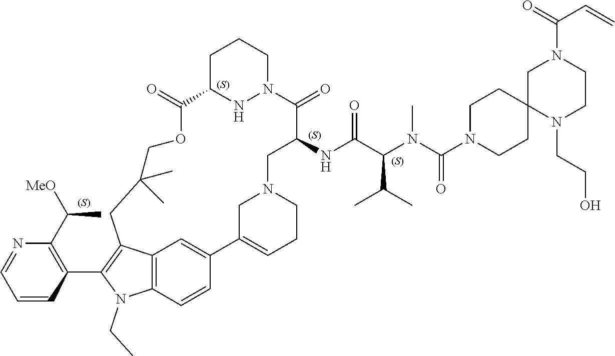 |
| A70 | 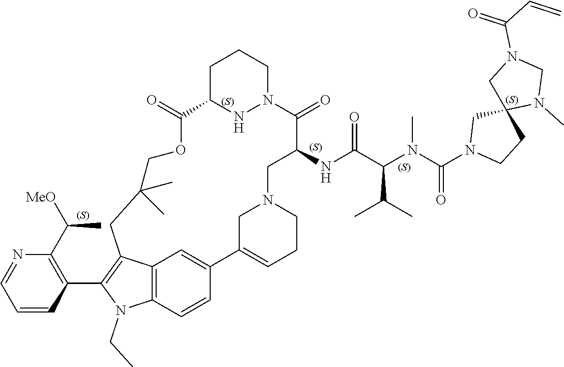 |
| A71 | 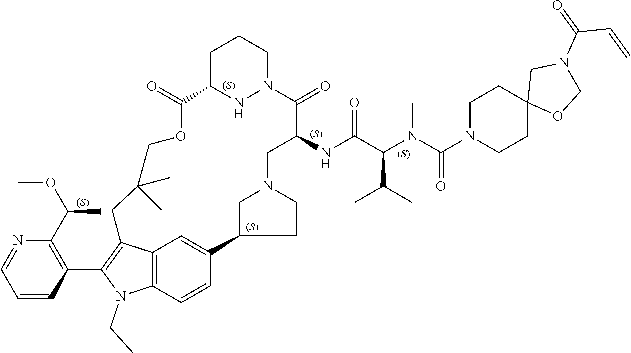 |
| A72 | 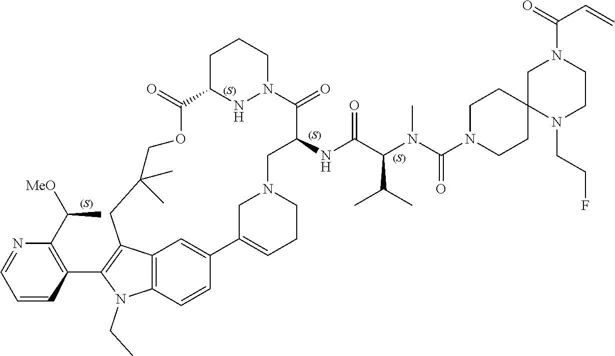 |
| A73 | 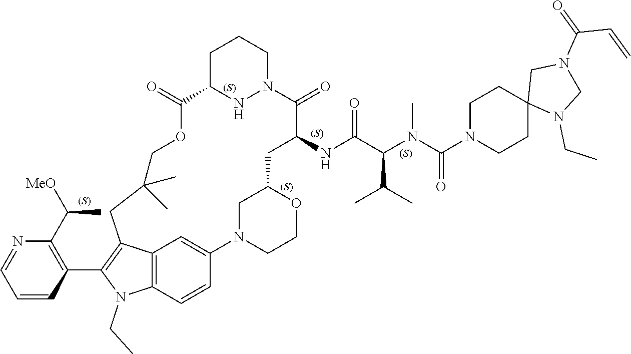 |
| A74 | 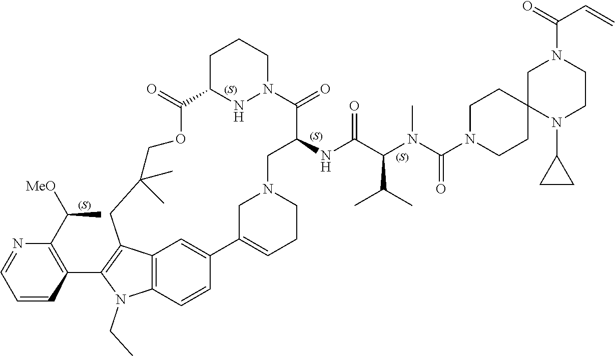 |
| A75 | 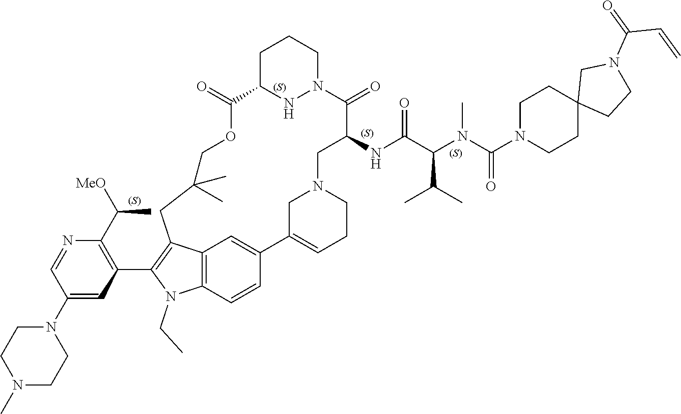 |
| A76 | 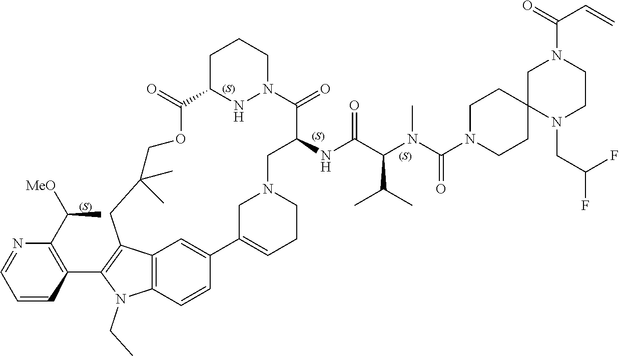 |
| A77 | 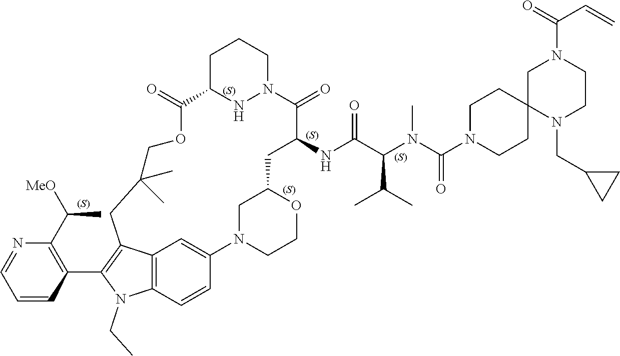 |
| A78 | 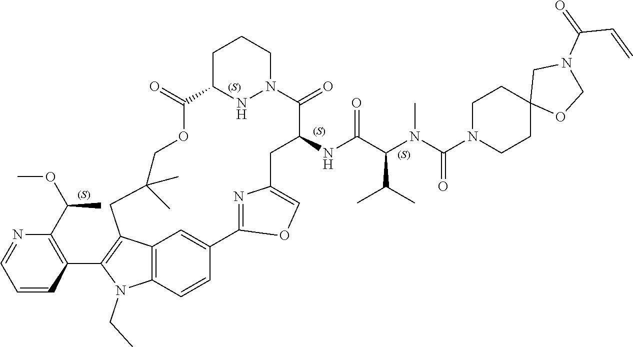 |
| A79 | 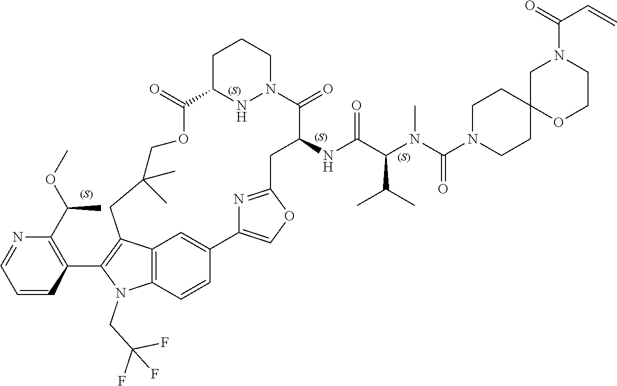 |
| A80 | 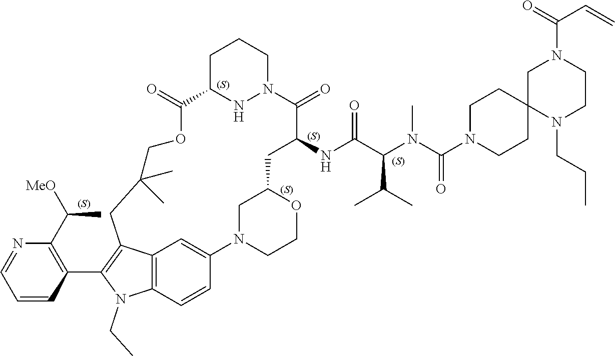 |
| A81 | 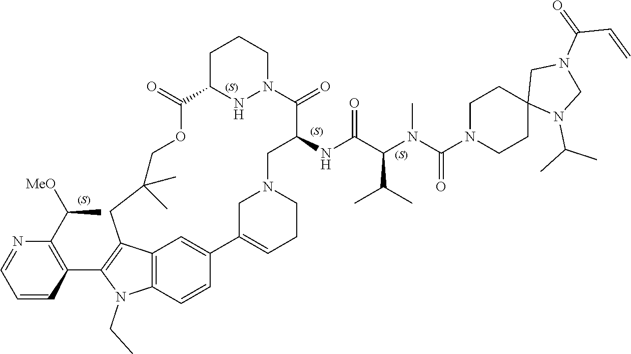 |
| A82 | 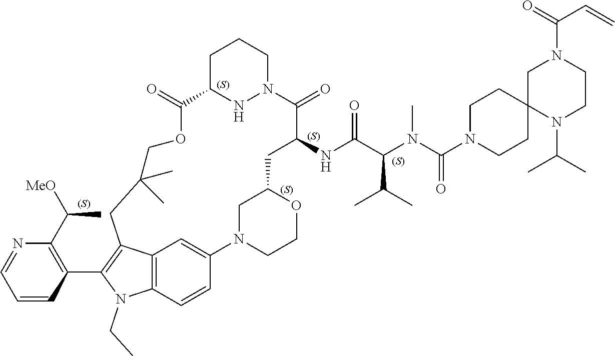 |
| A83 | 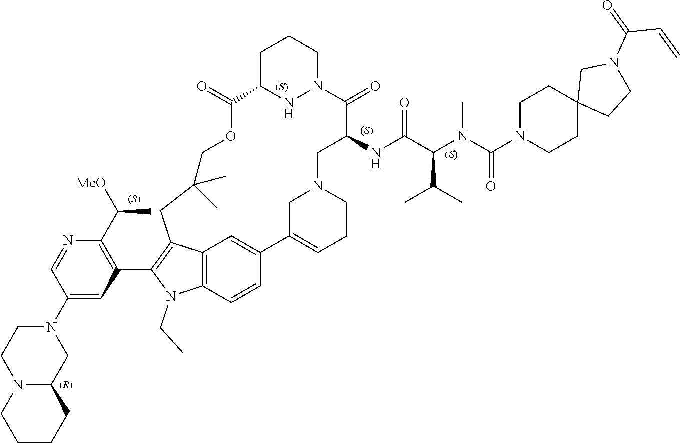 |
| A84 | 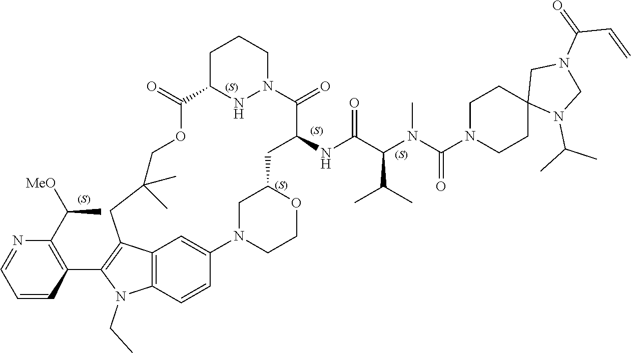 |
| A85 | 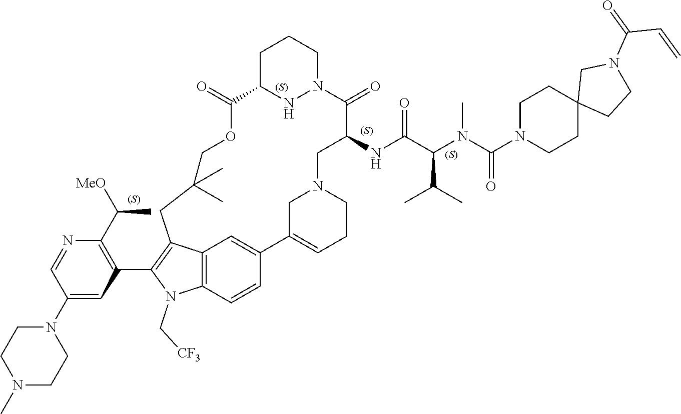 |
| A86 | 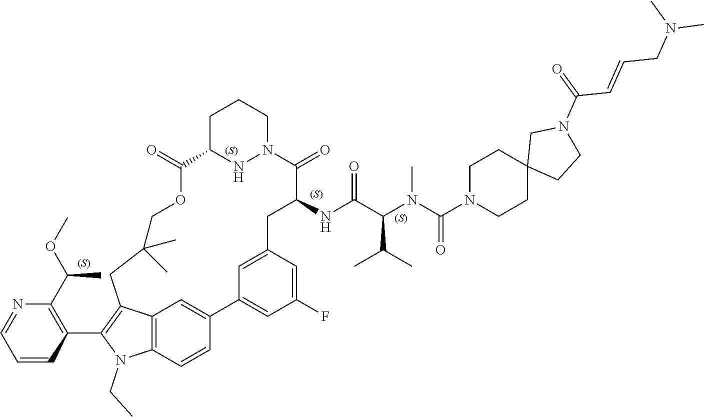 |
| A87 | 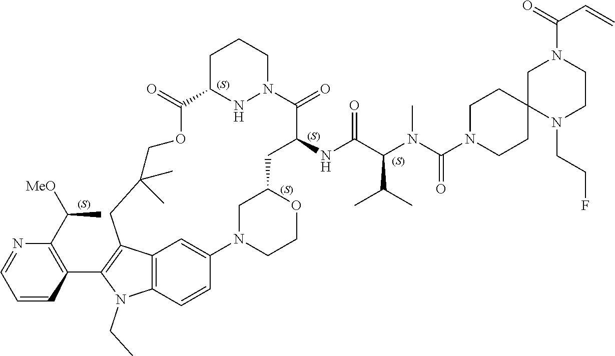 |
| A88 | 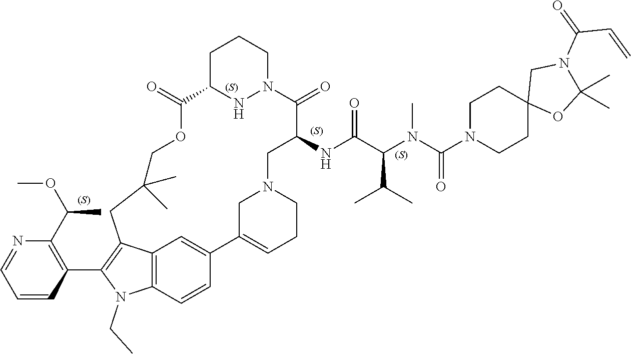 |
| A89 | 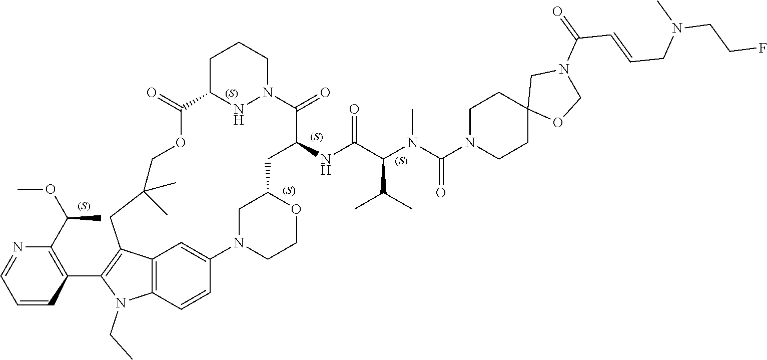 |
| A90 | 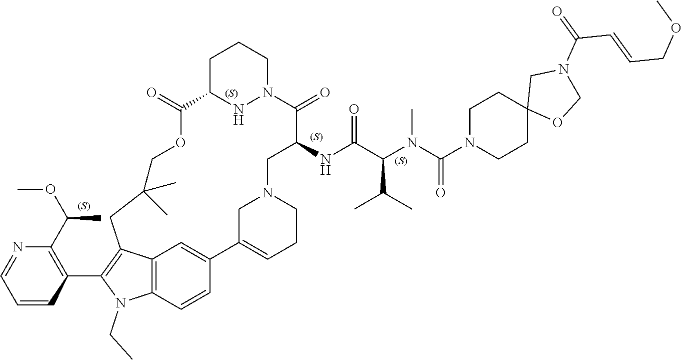 |
| A91 | 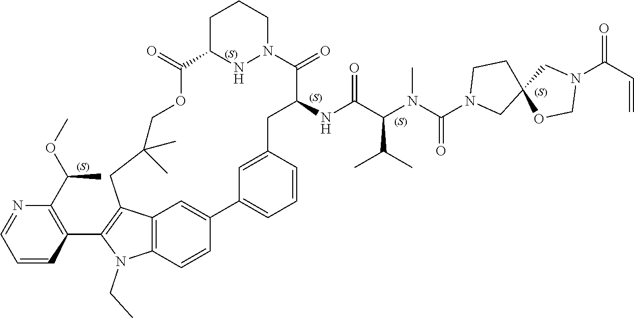 |
| A92 | 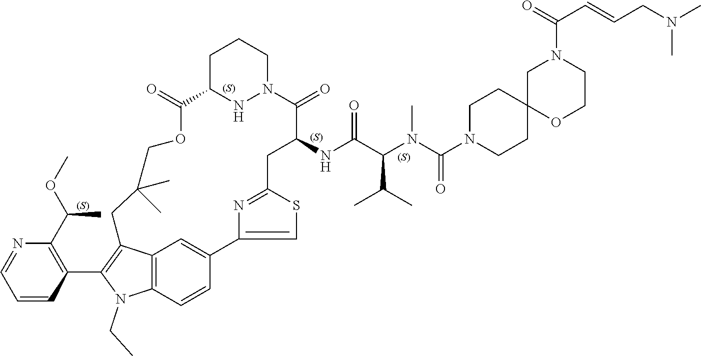 |
| A93 | 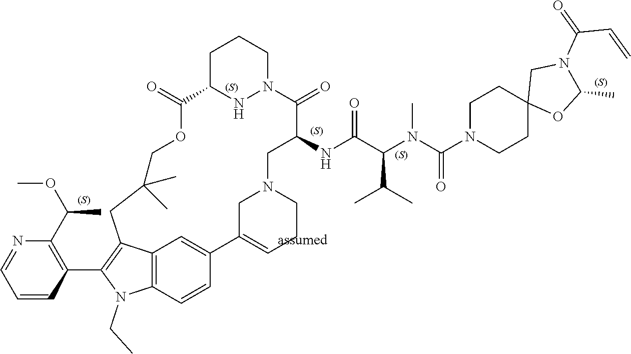 |
| A94 | 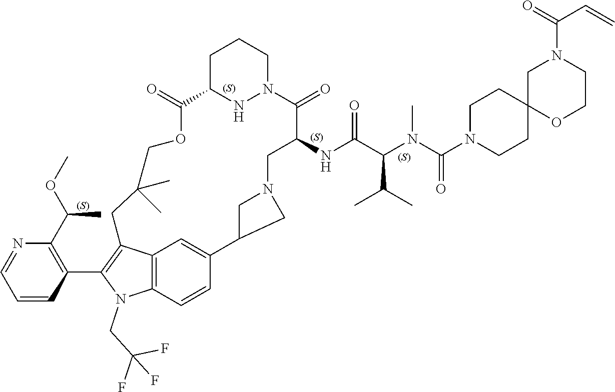 |
| A95 | 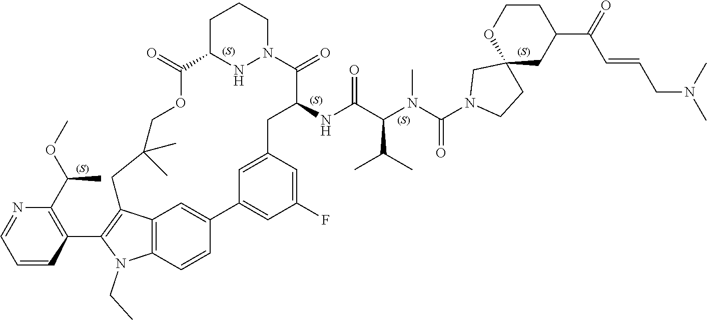 |
| A96 | 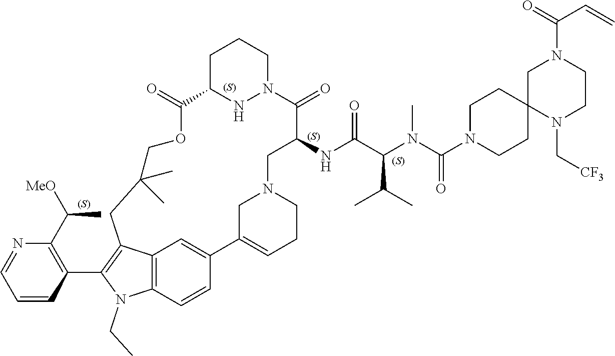 |
| A97 | 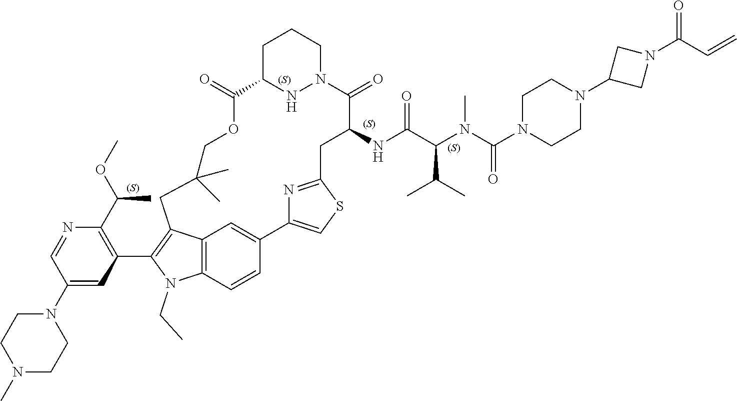 |
| A98 | 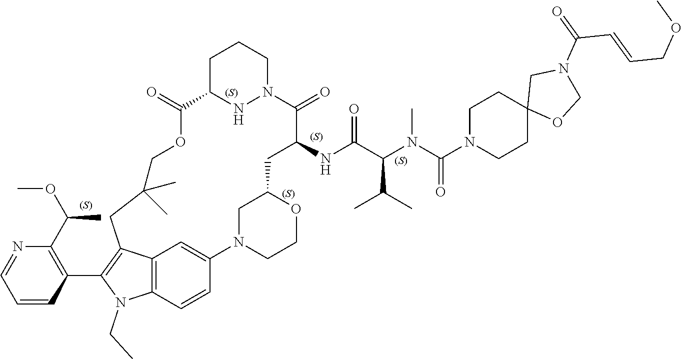 |
| A99 | 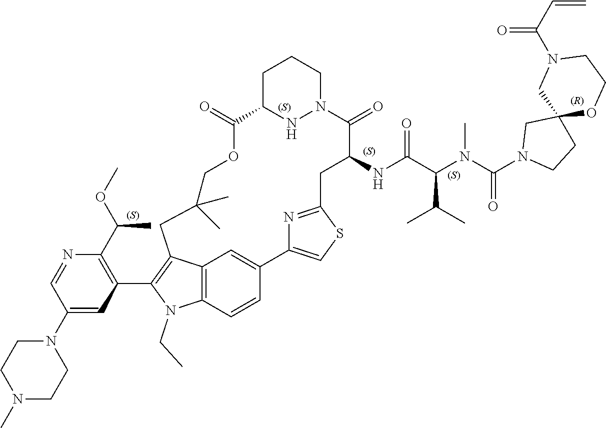 |
| A100 | 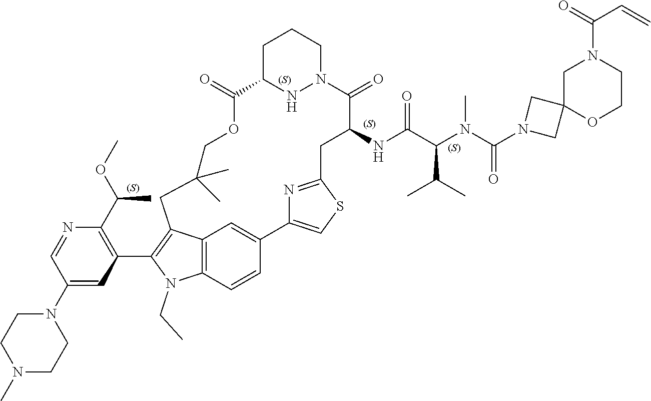 |
| A101 | 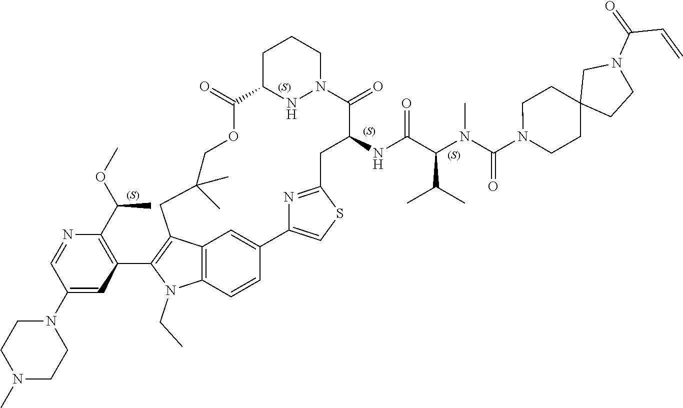 |
| A102 | 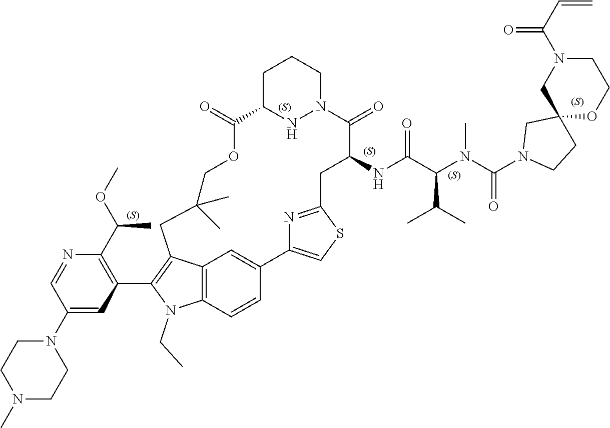 |
| A103 | 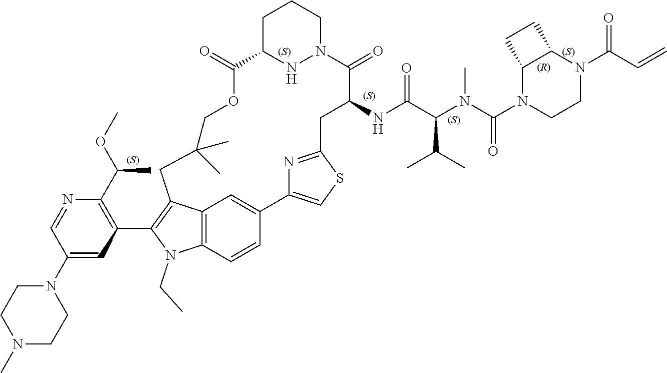 |
| A104 | 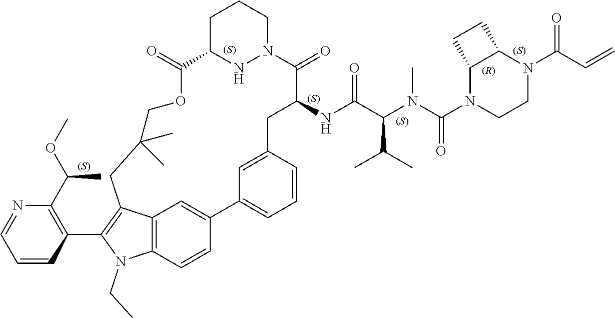 |
| A105 | 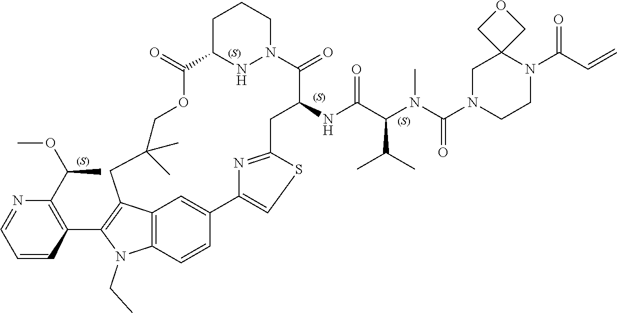 |
| A106 | 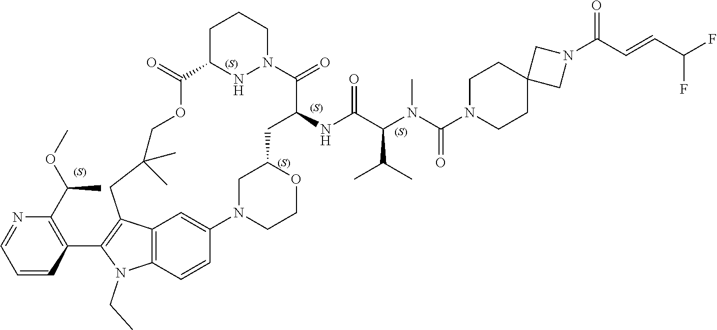 |
| A107 | 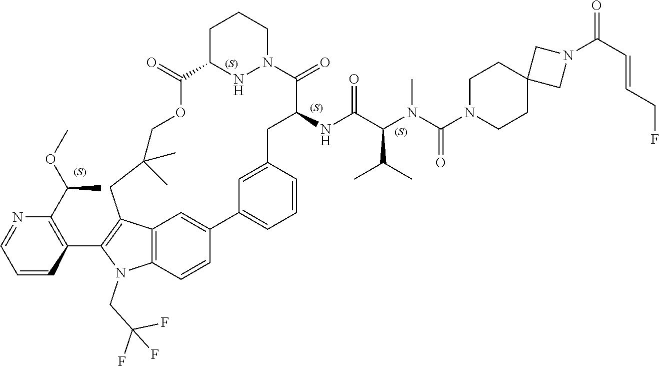 |
| A108 | 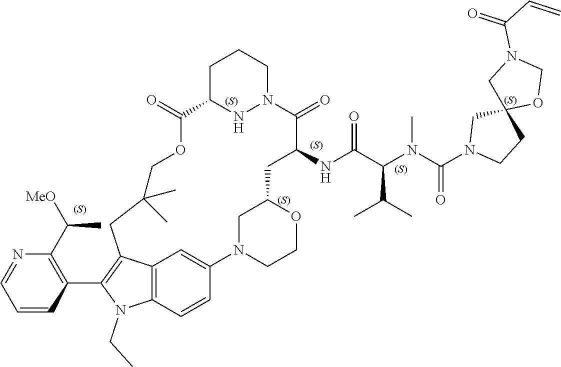 |
| A109 | 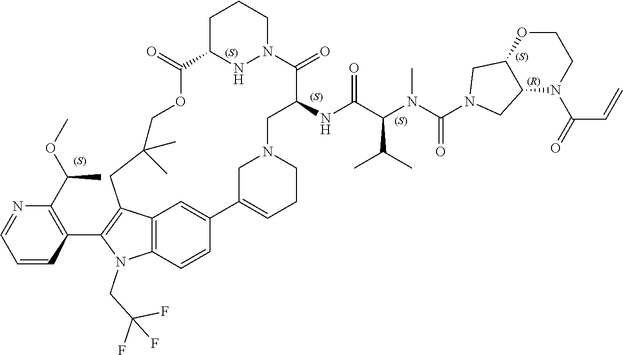 |
| A110 | 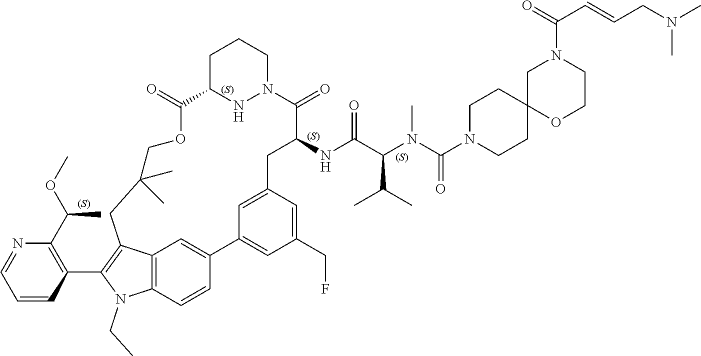 |
| A111 | 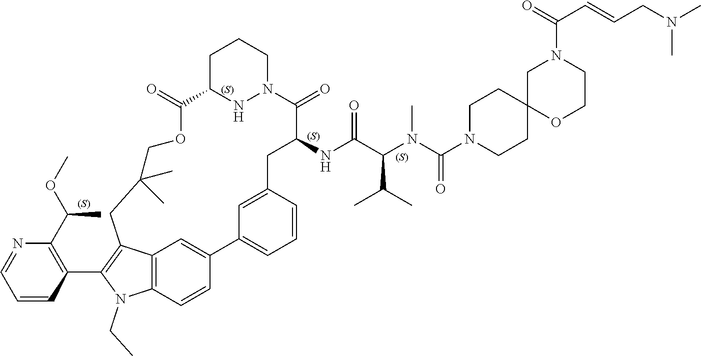 |
| A112 | 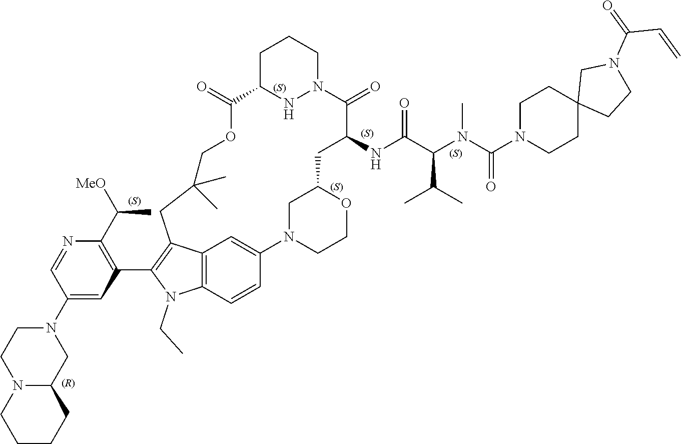 |
| A113 | 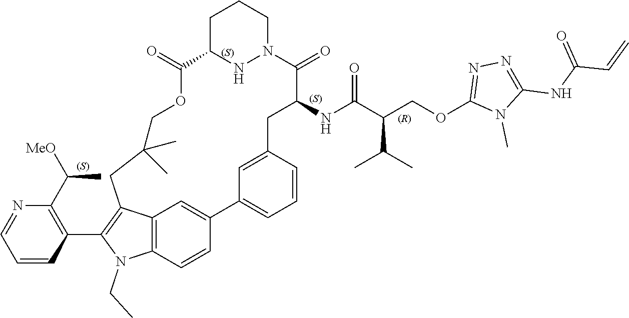 |
| A114 | 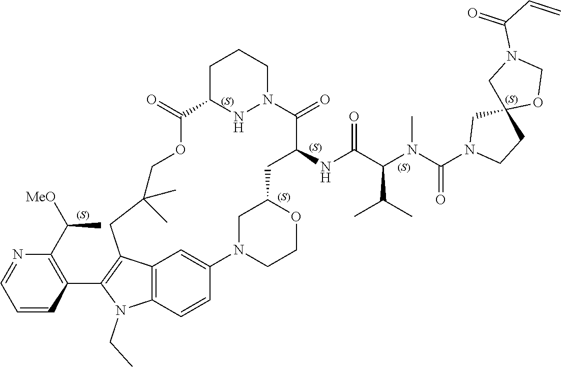 |
| A115 | 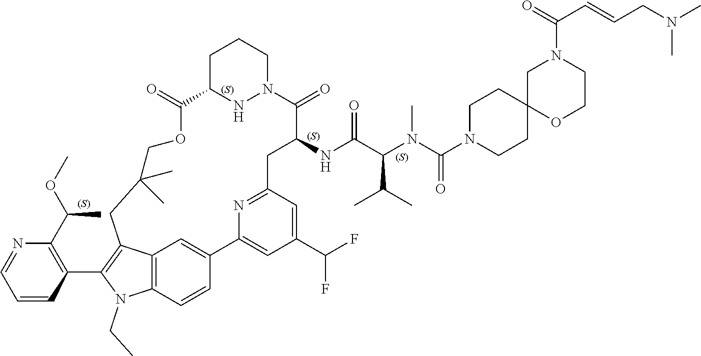 |
| A116 | 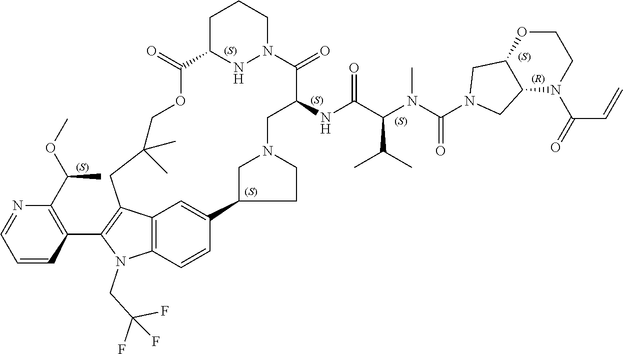 |
| A117 | 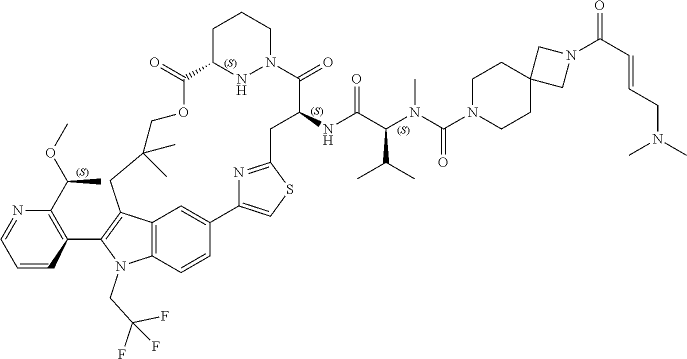 |
| A118 | 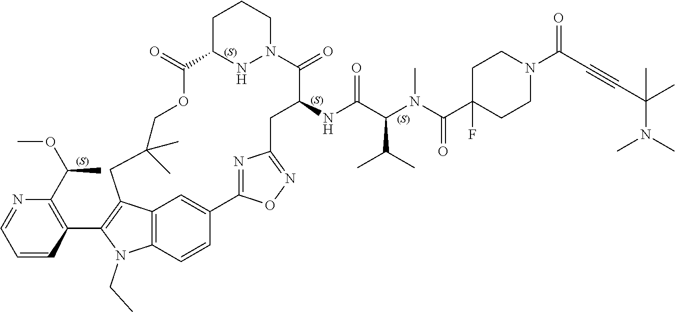 |
| A119 | 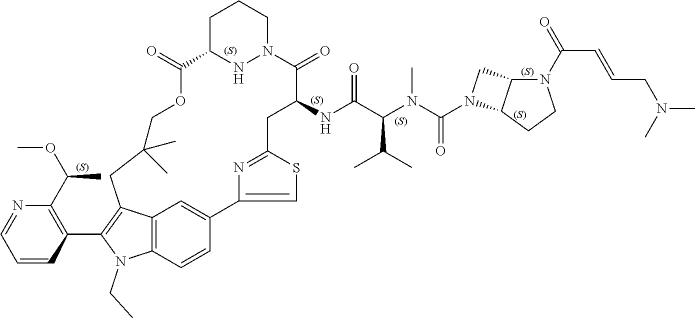 |
| A120 | 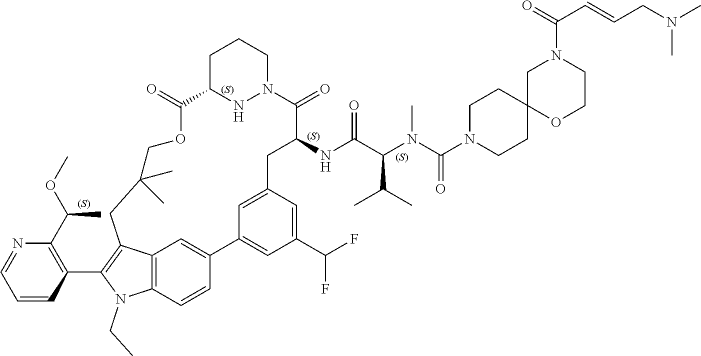 |
| A121 | 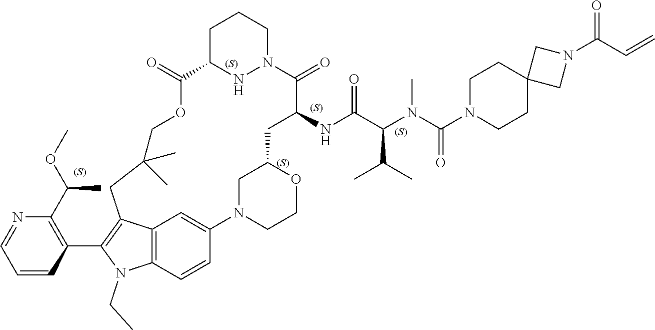 |
| A122 | 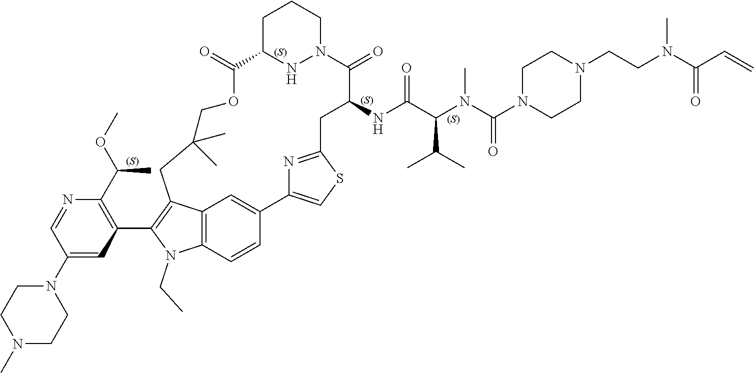 |
| A123 | 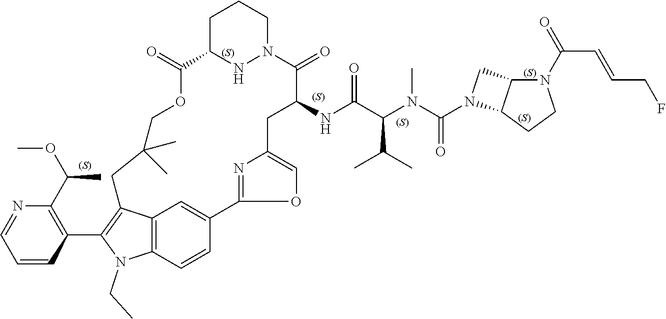 |
| A124 | 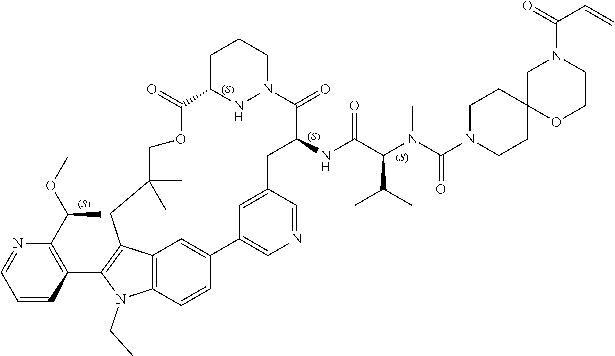 |
| A125 | 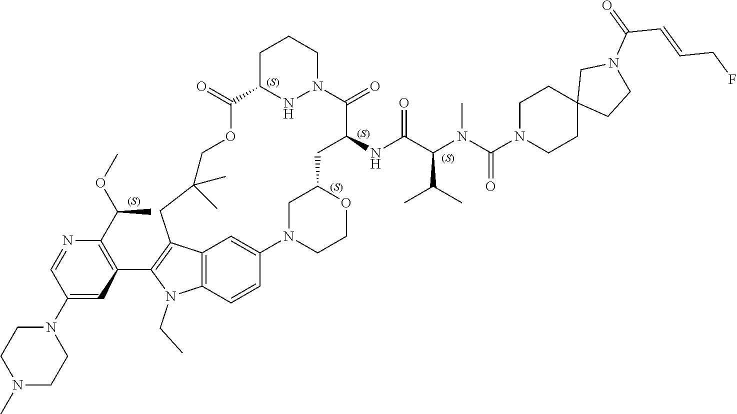 |
| A126 | 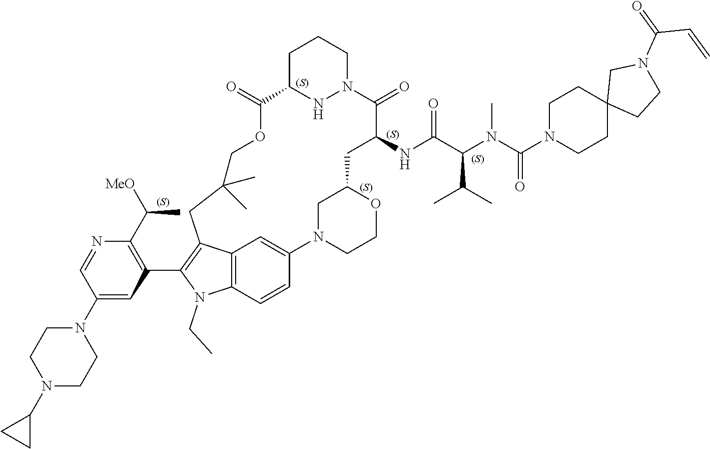 |
| A127 | 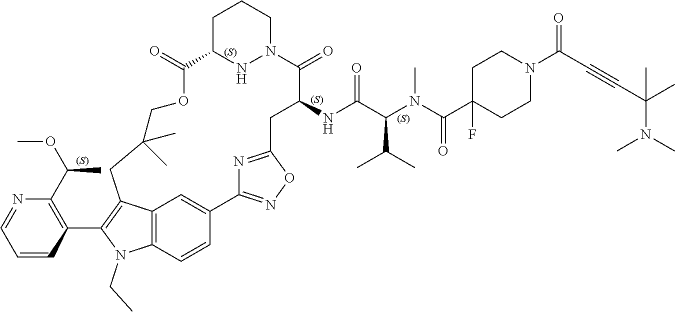 |
| A128 | 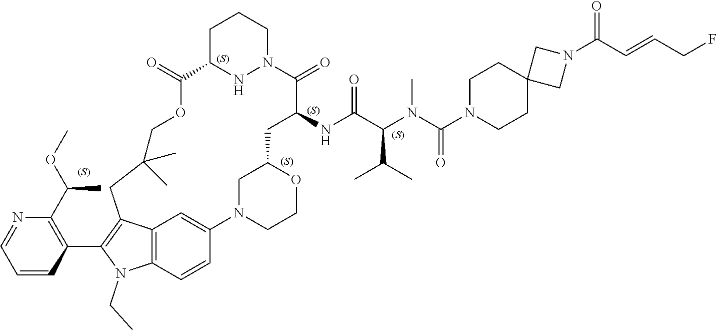 |
| A129 | 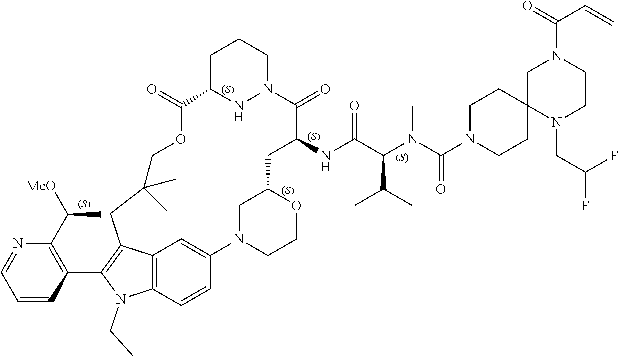 |
| A130 | 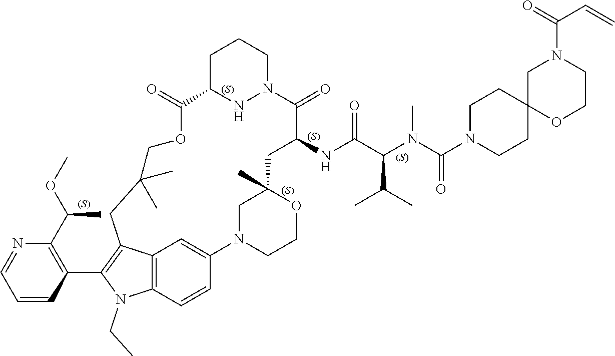 |
| A131 | 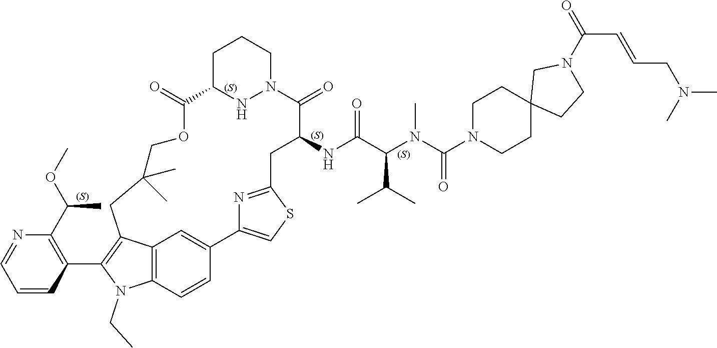 |
| A132 | 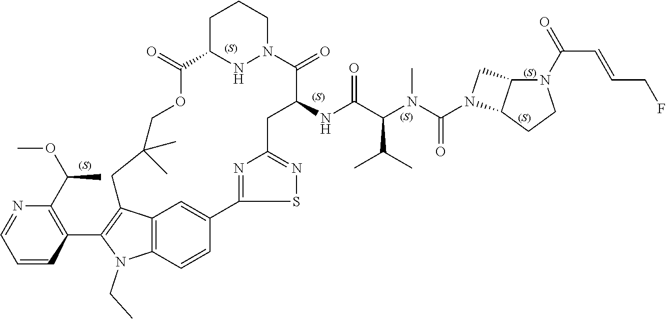 |
| A133 | 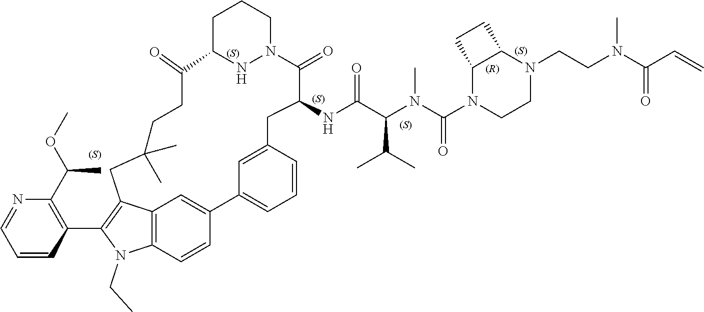 |
| A134 | 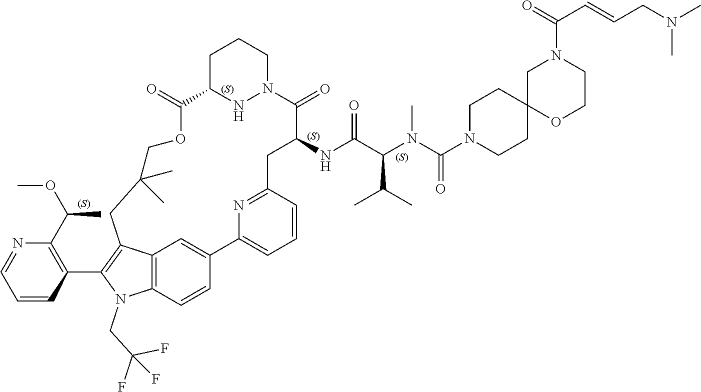 |
| A135 | 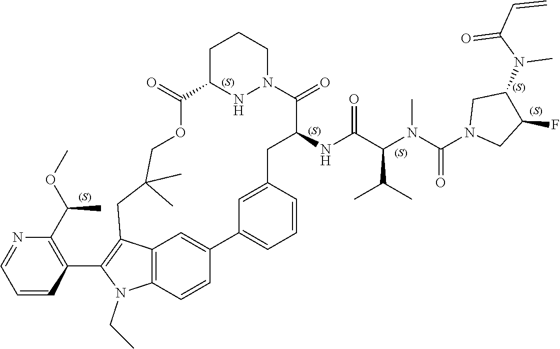 |
| A136 | 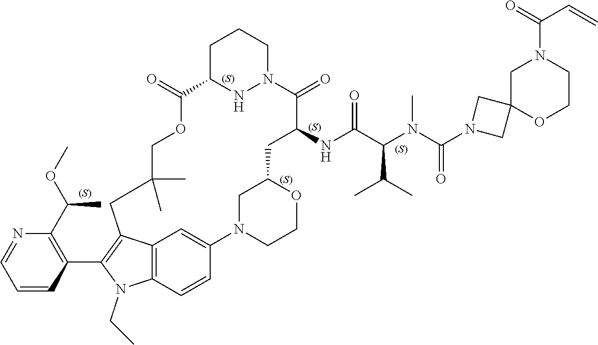 |
| A137 | 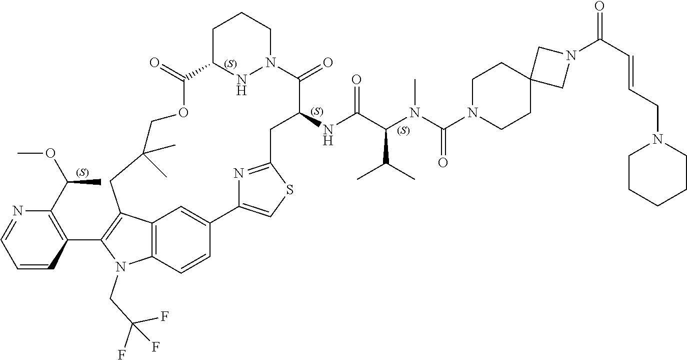 |
| A138 | 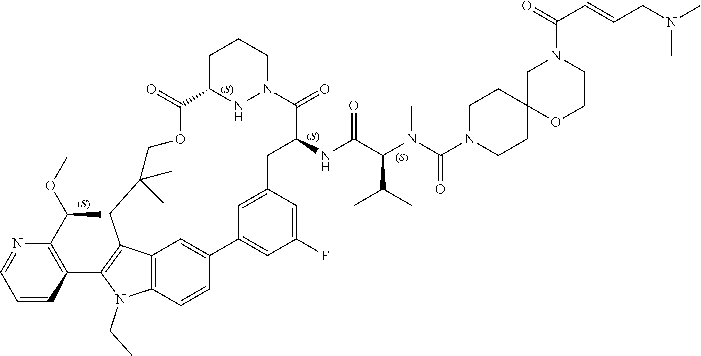 |
| A139 | 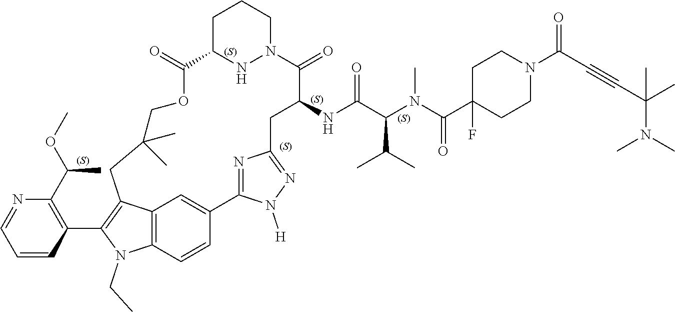 |
| A140 | 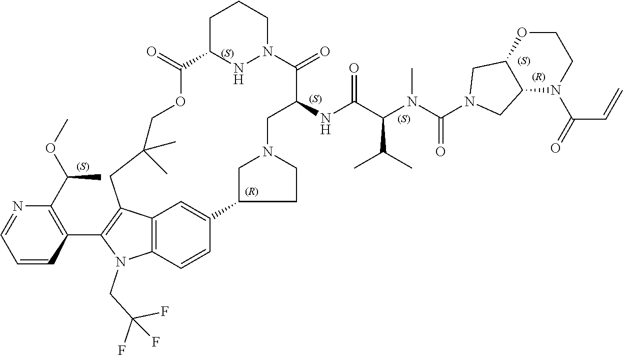 |
| A141 | 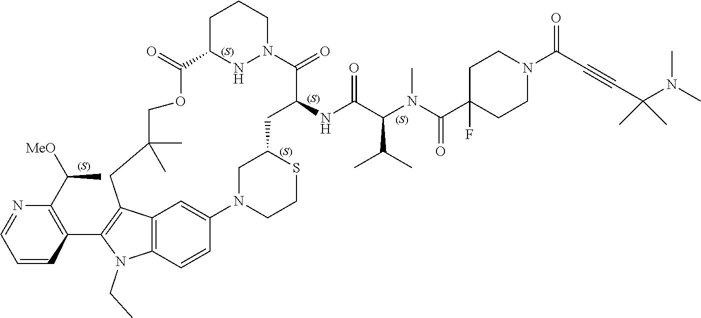 |
| A142 | 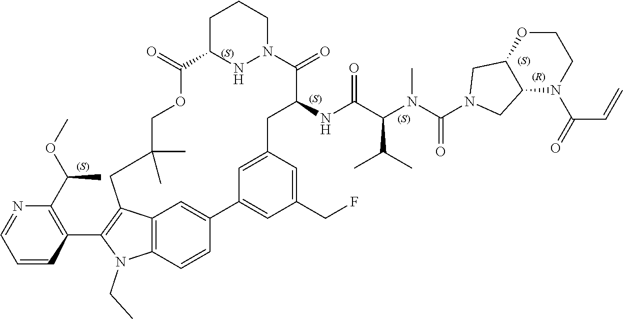 |
| A143 | 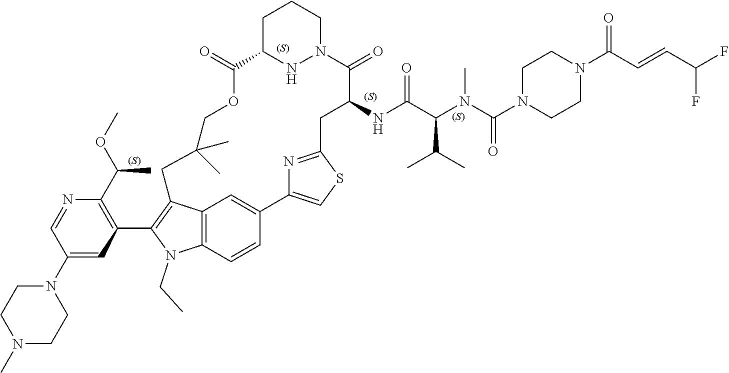 |
| A144 | 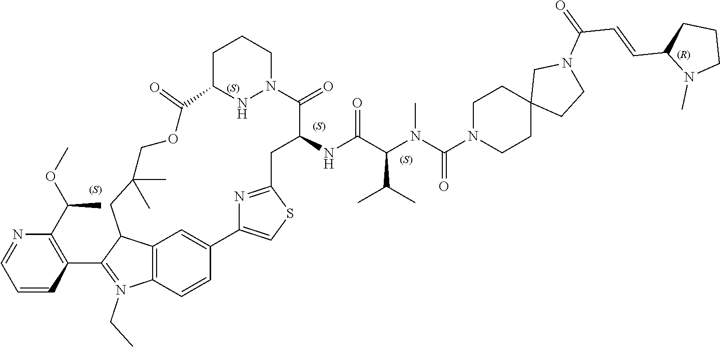 |
| A145 | 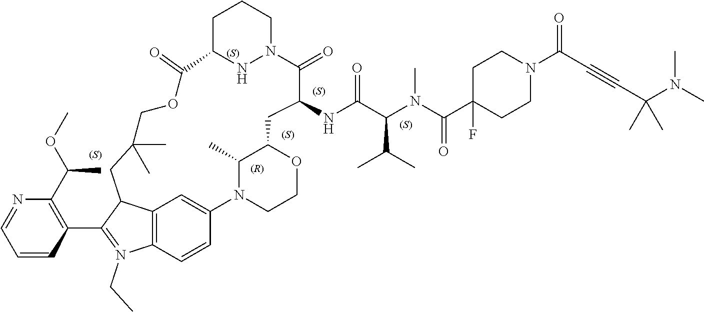 |
| A146 | 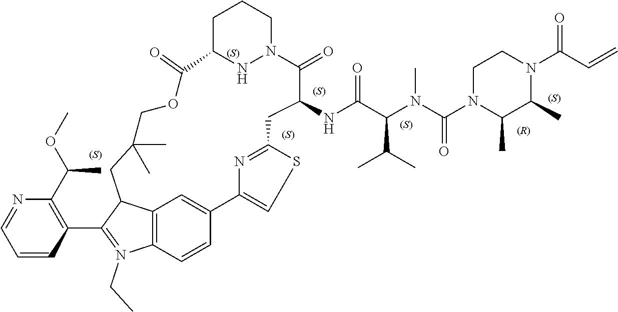 |
| A147 | 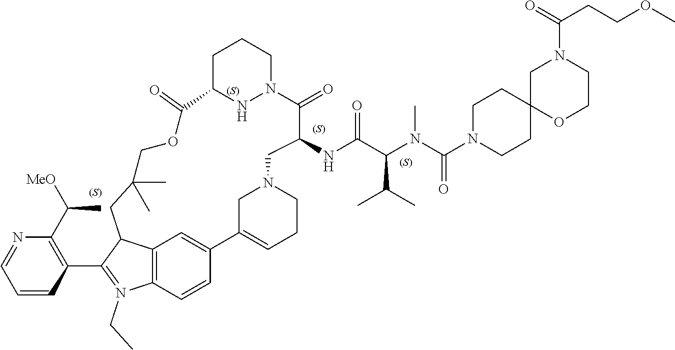 |
| A148 | 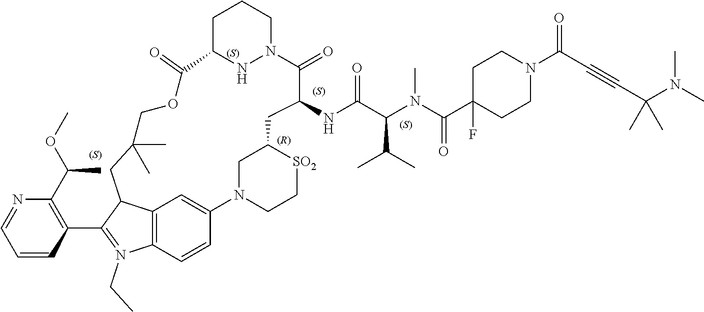 |
| A149 | 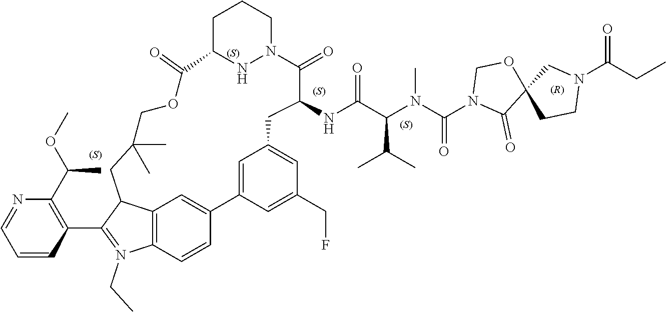 |
| A150 | 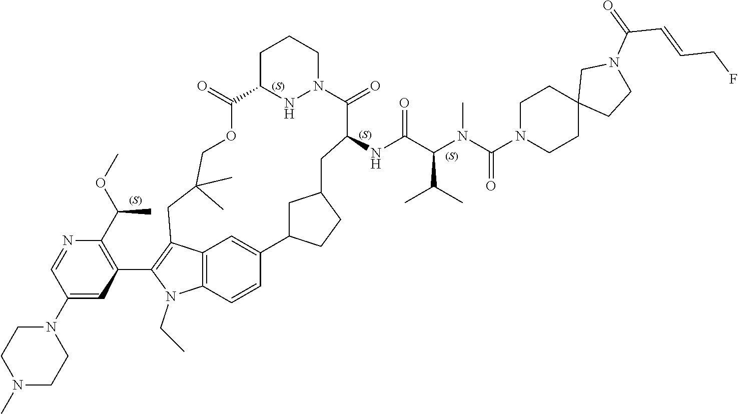 |
| A151 | 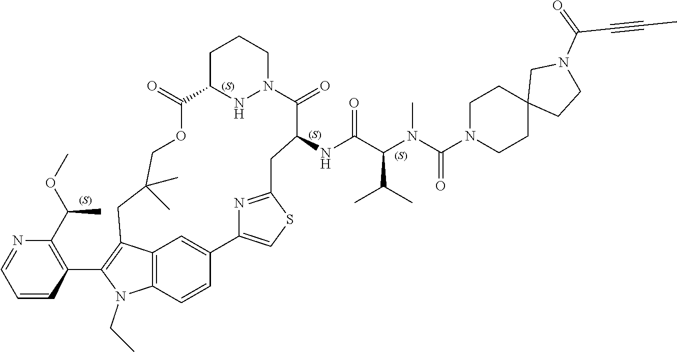 |
| A152 | 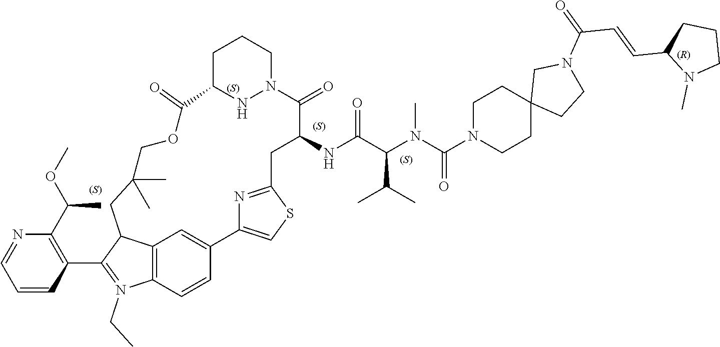 |
| A153 | 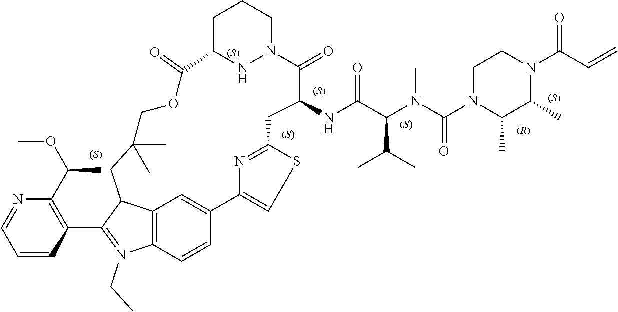 |
| A154 | 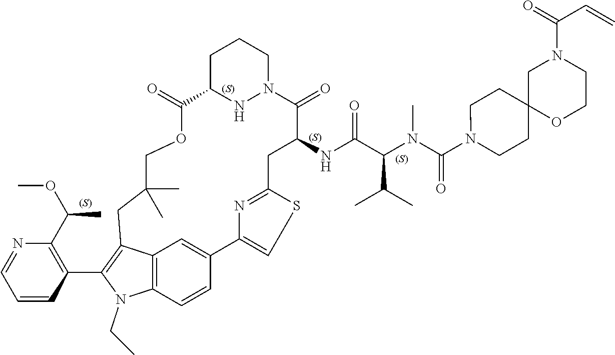 |
| A155 | 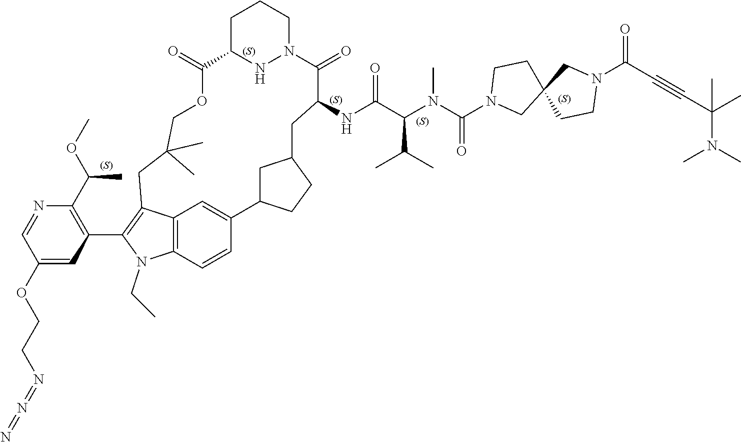 |
| A156 | 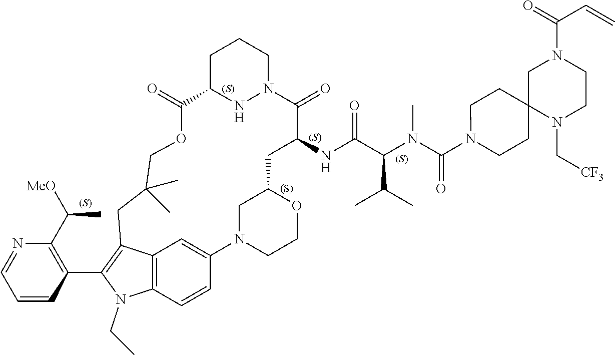 |
| A157 | 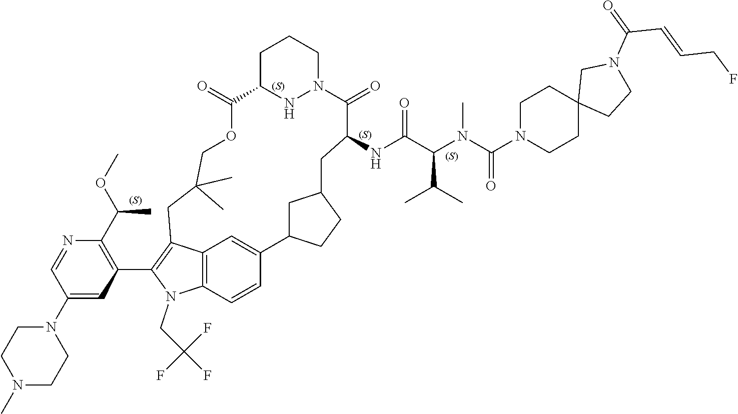 |
| A158 | 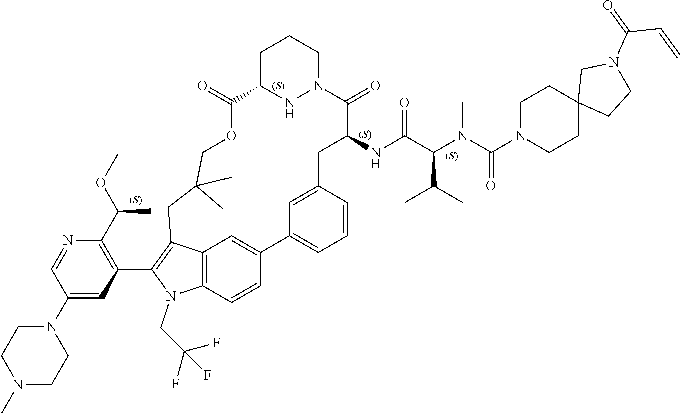 |
| A159 | 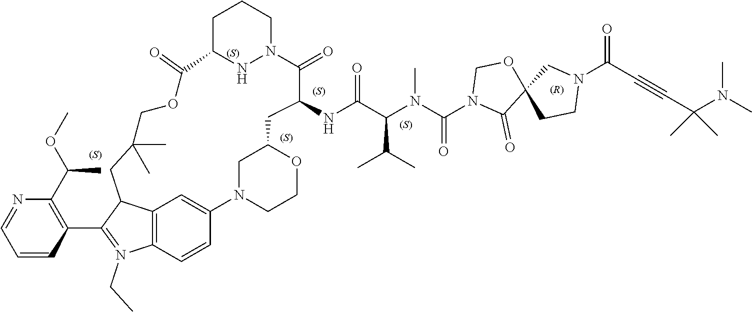 |
| A160 | 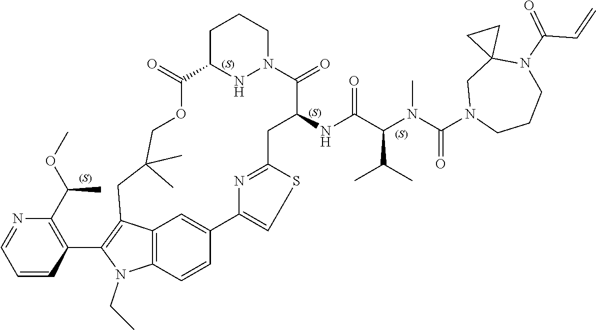 |
| A161 | 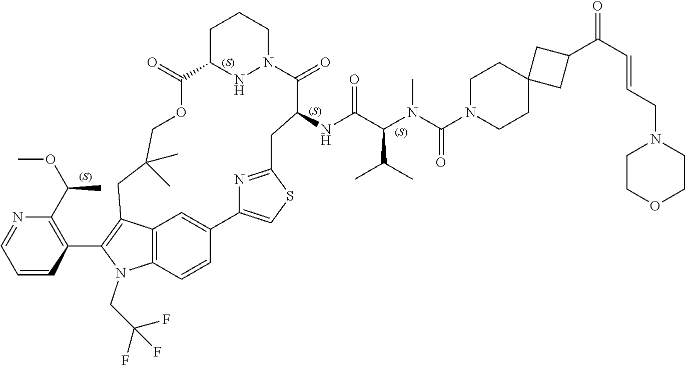 |
| A162 | 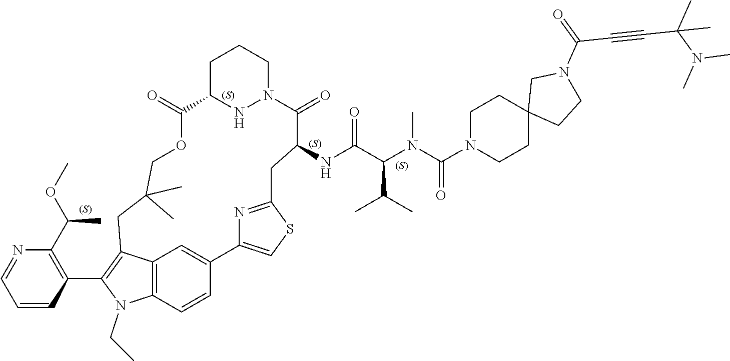 |
| A163 | 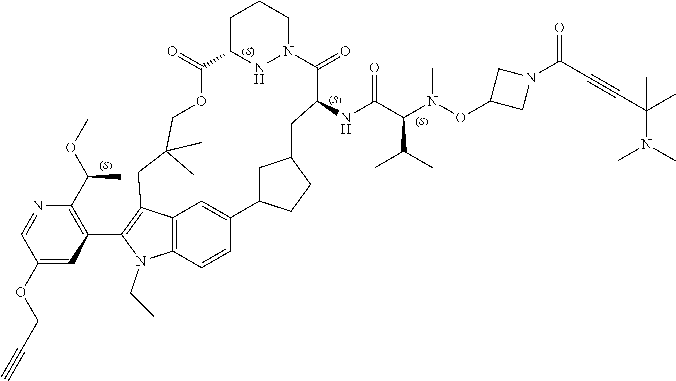 |
| A164 | 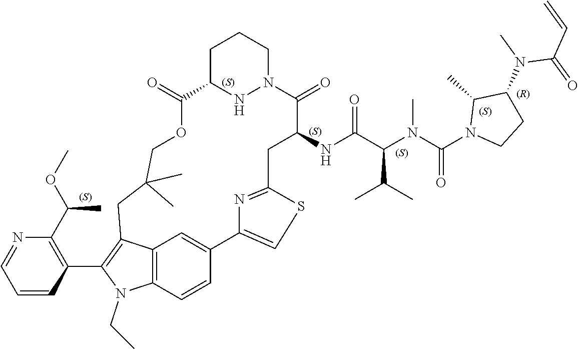 |
| A165 | 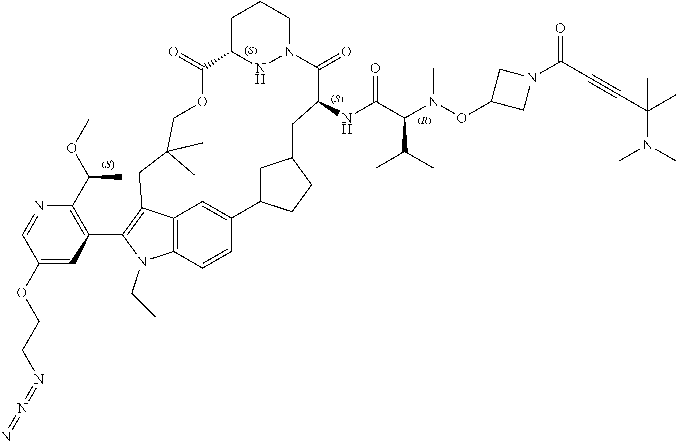 |
| A166 | 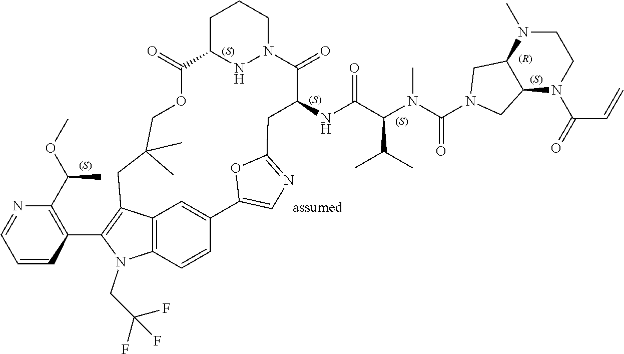 |
| A167 | 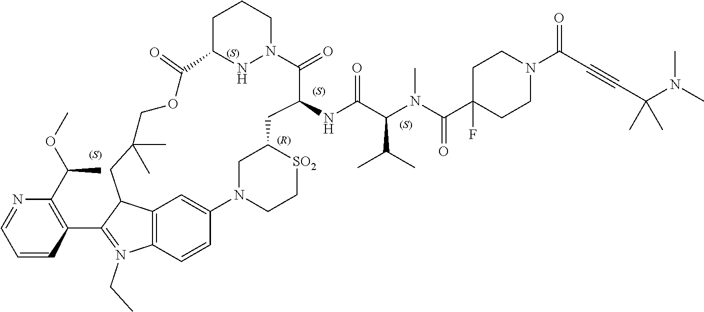 |
| A168 | 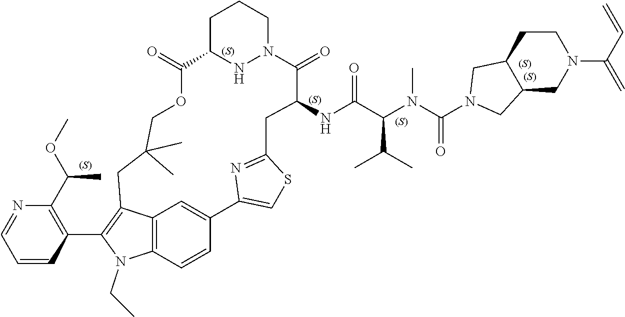 |
| A169 | 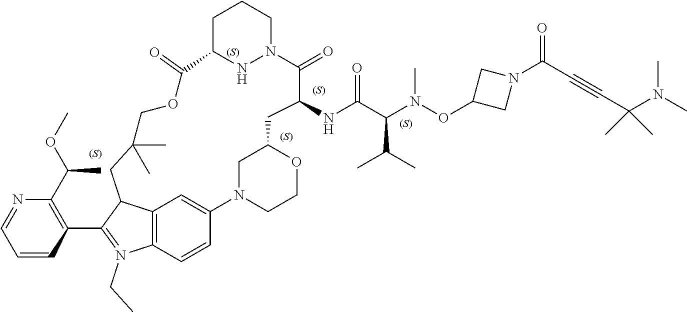 |
| A170 | 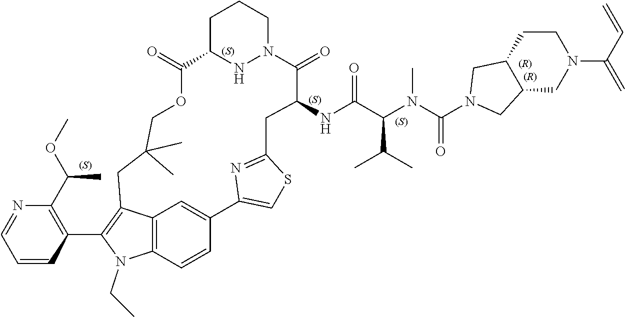 |
| A171 | 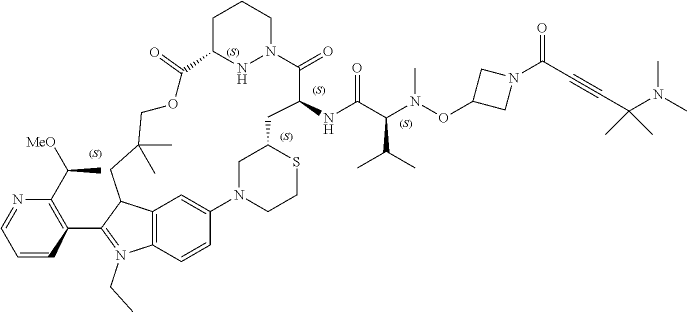 |
| A172 | 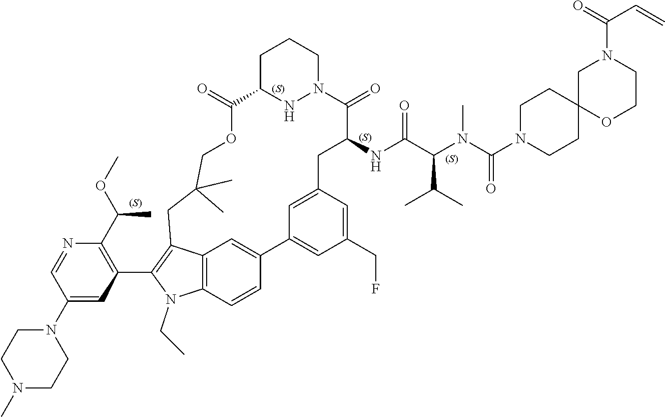 |
| A173 | 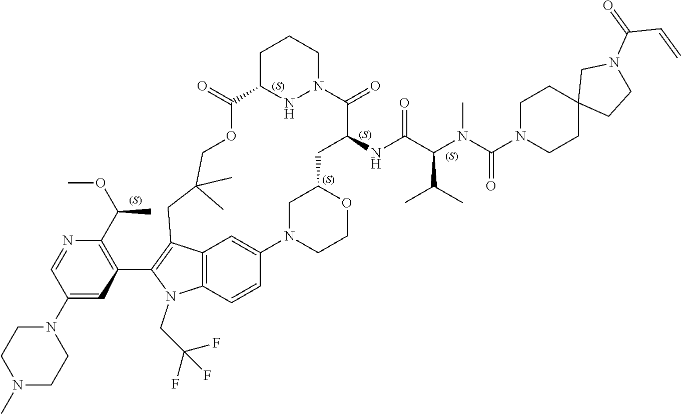 |
| A174 | 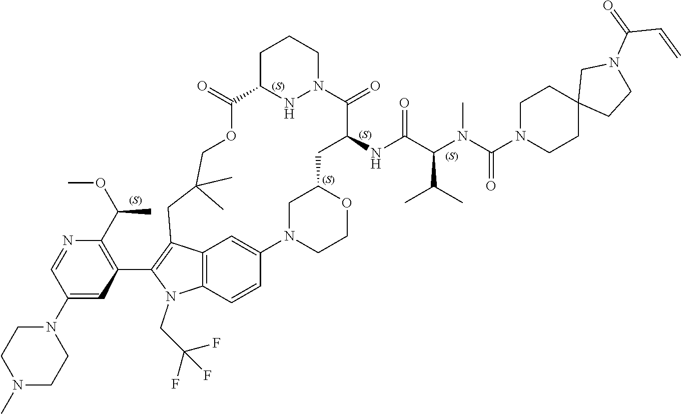 |
| A175 | 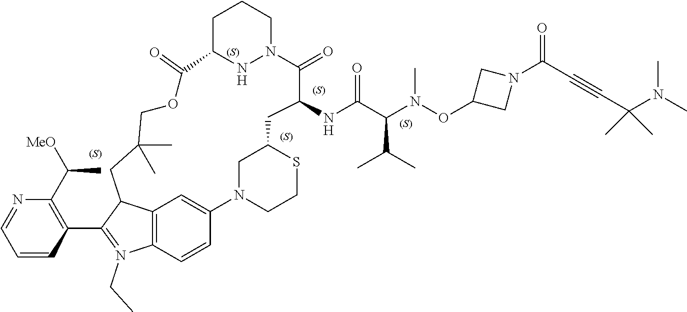 |
| A176 | 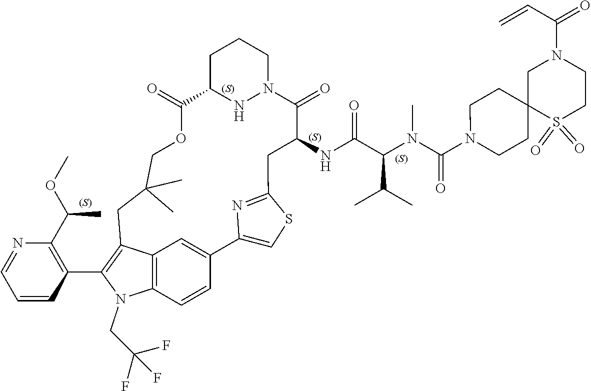 |
| A177 | 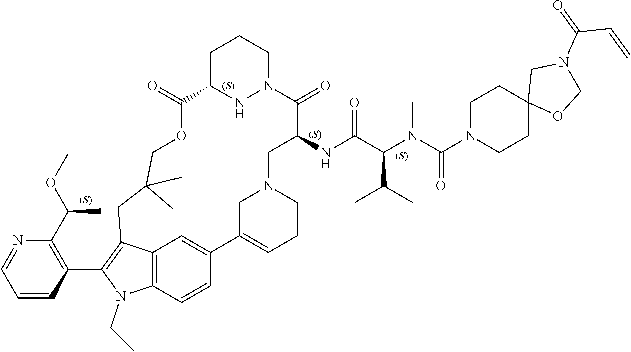 |
| A178 | 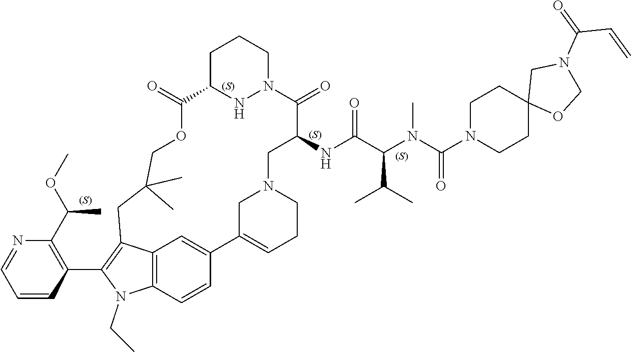 |
| A179 | 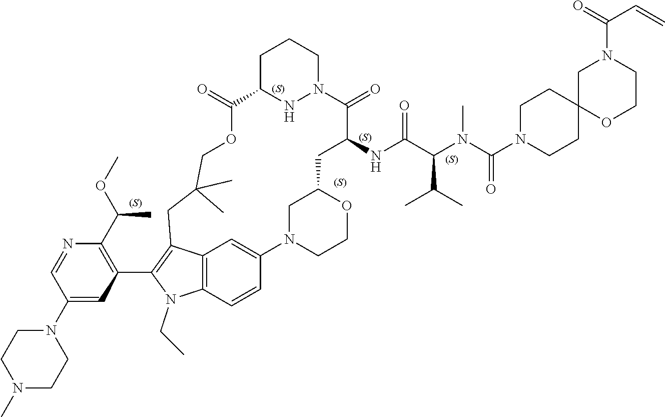 |
| A180 | 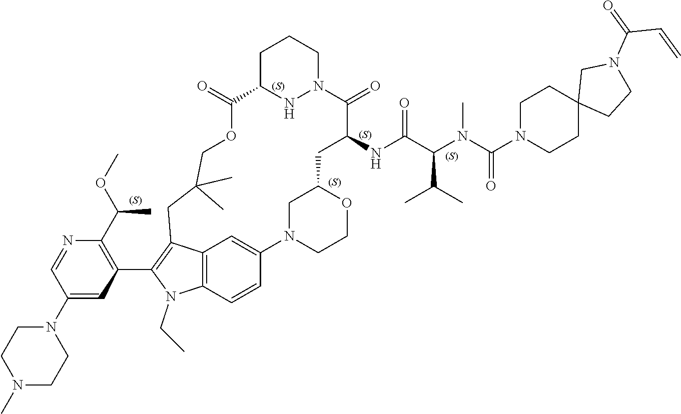 |
| A181 | 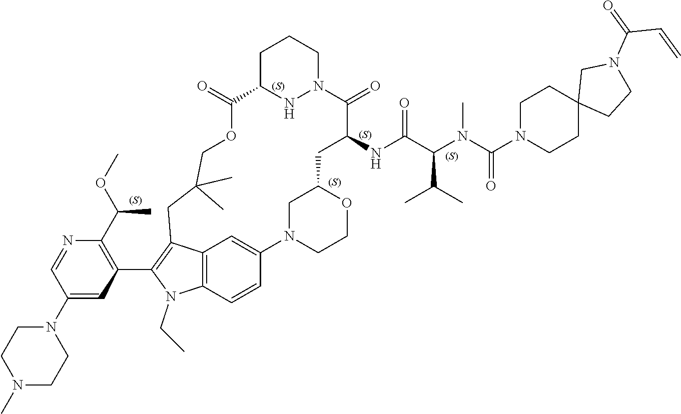 |
| A182 | 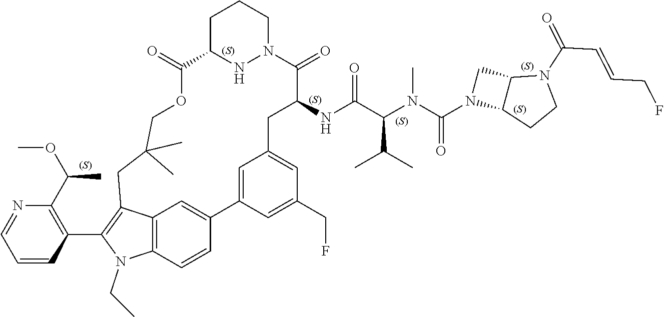 |
| A183 | 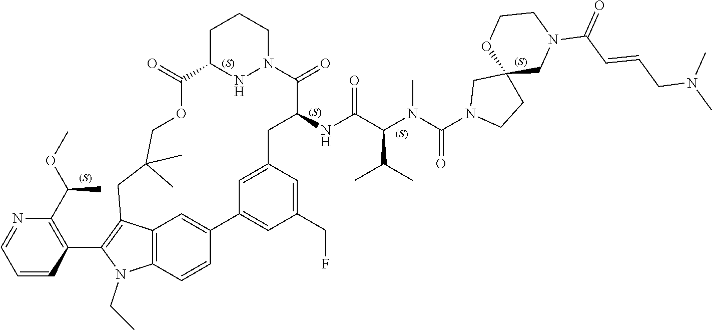 |
| A184 | 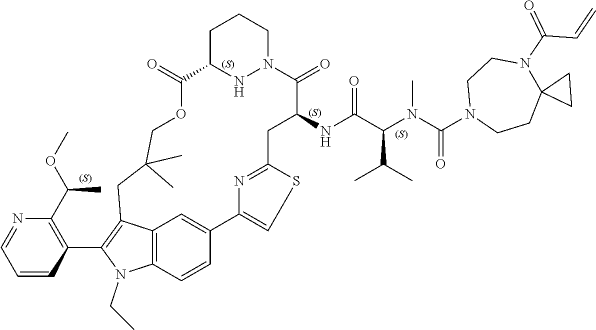 |
| A185 | 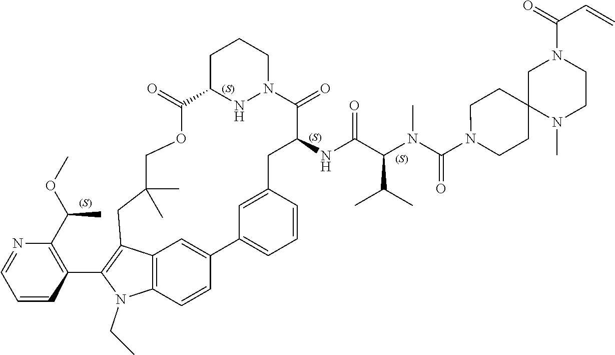 |
| A186 | 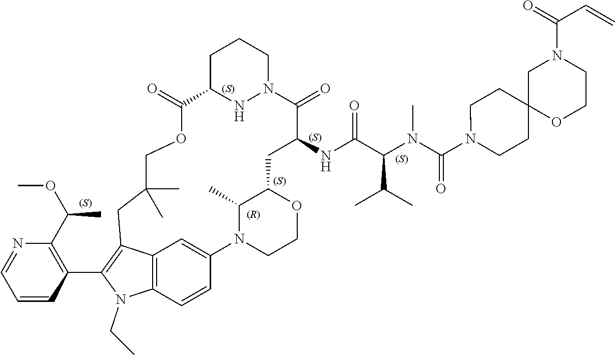 |
| A187 | 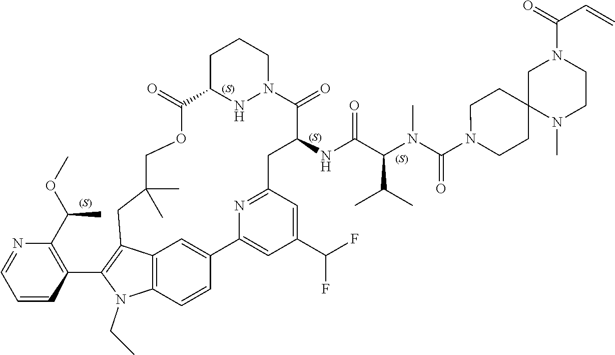 |
| A188 | 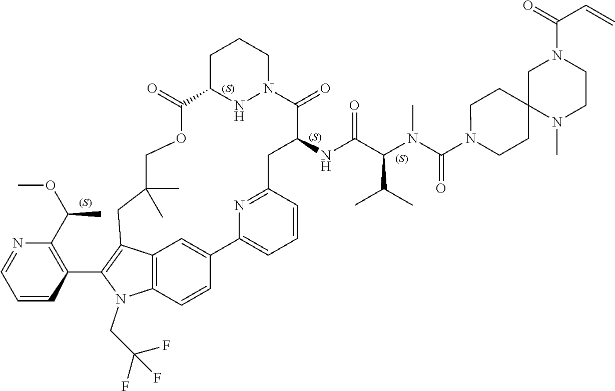 |
| A189 | 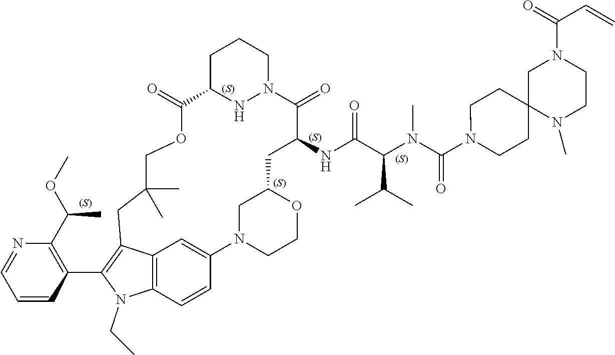 |
| A190 | 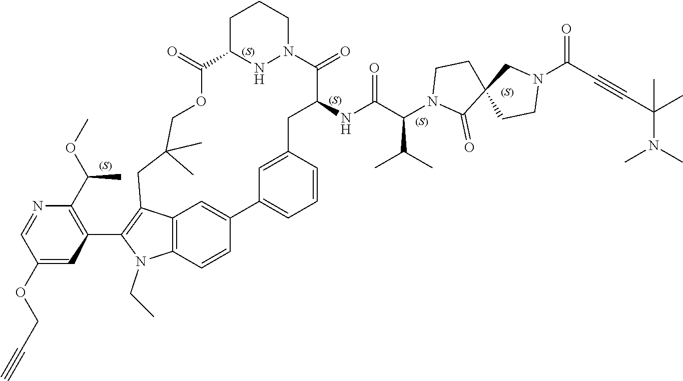 |
| A191 | 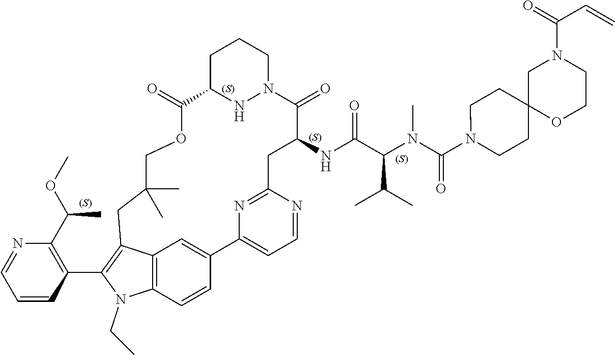 |
| A192 | 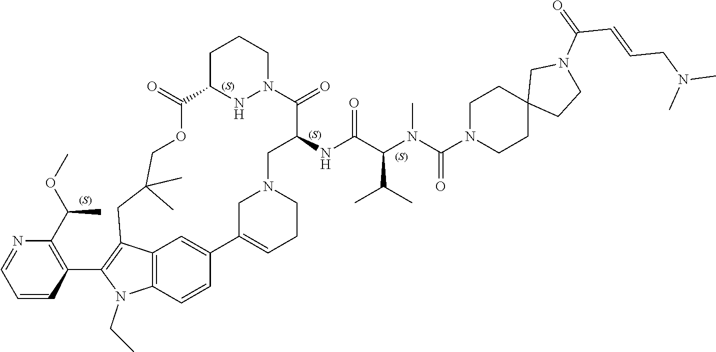 |
| A193 | 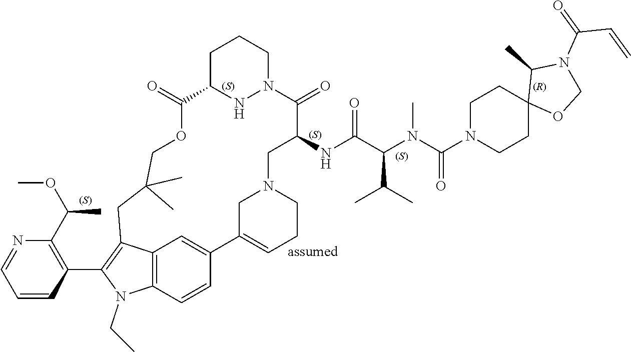 |
| A194 | 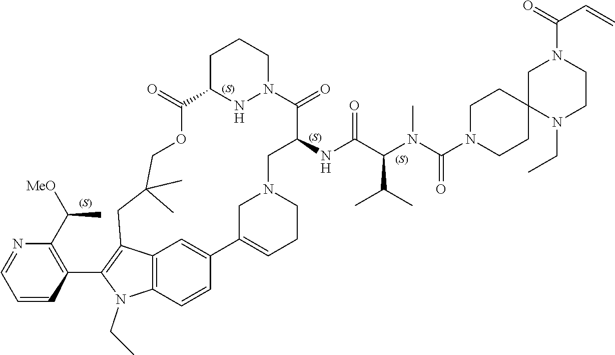 |
| A195 | 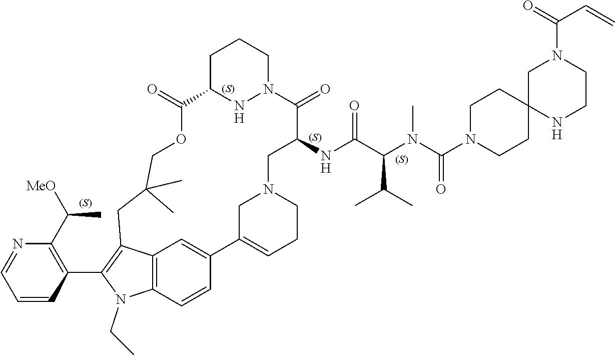 |
| A196 | 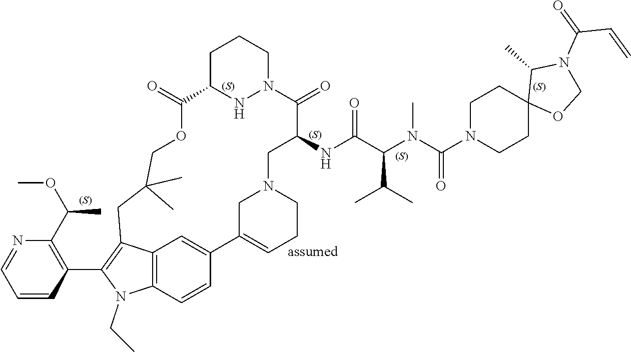 |
| A197 | 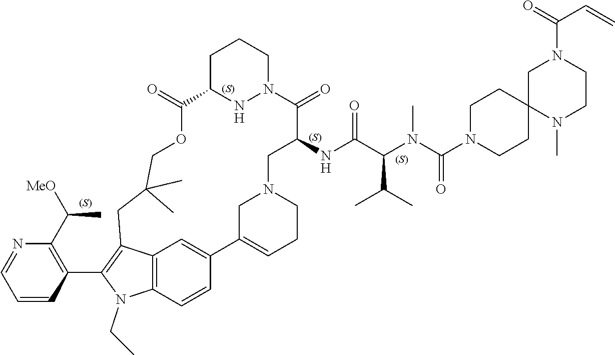 |
| A198 | 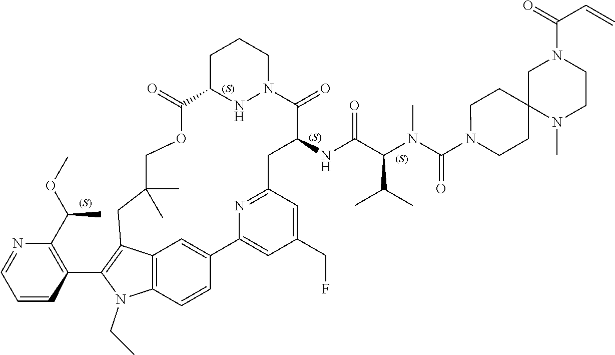 |
| A199 | 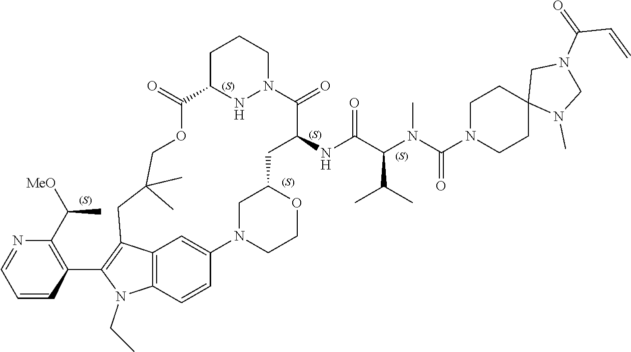 |
| A200 | 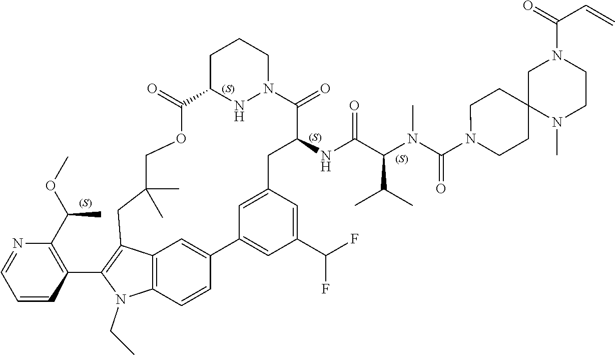 |
| A201 | 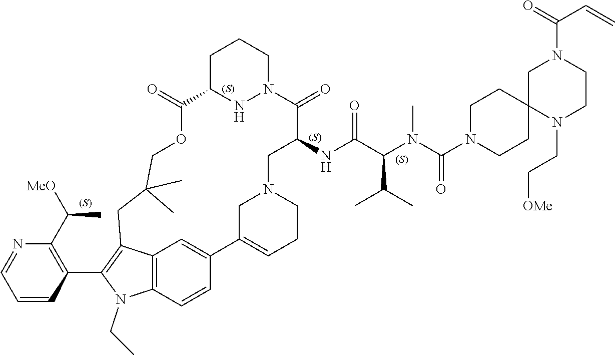 |
| A202 | 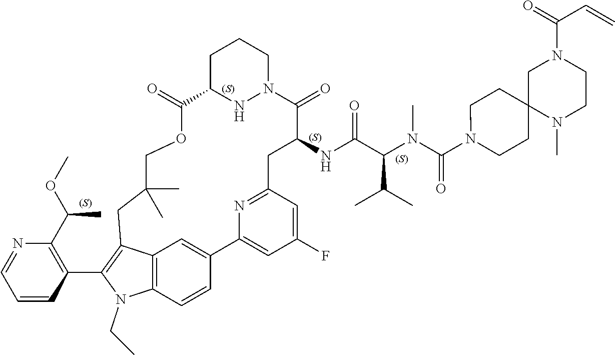 |
| A203 | 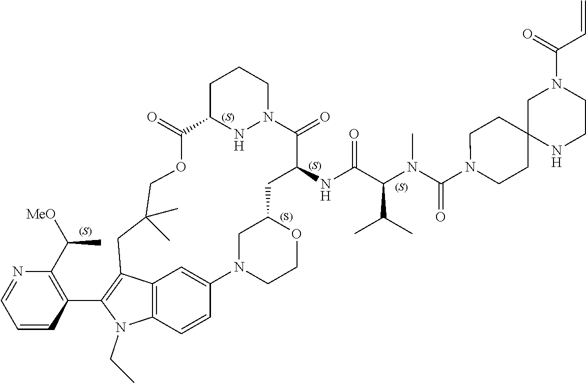 |
| A204 | 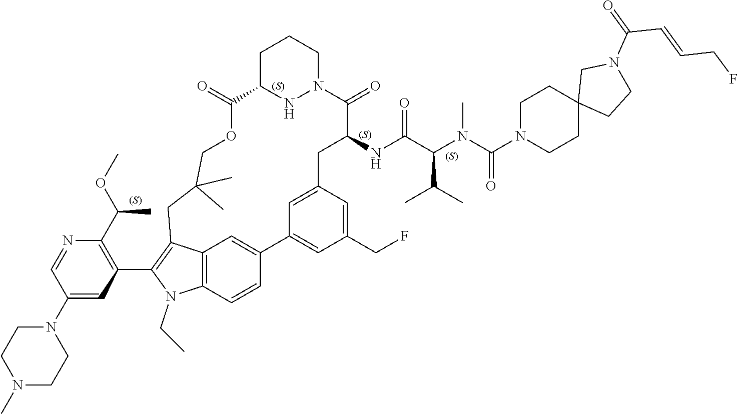 |
| A205 | 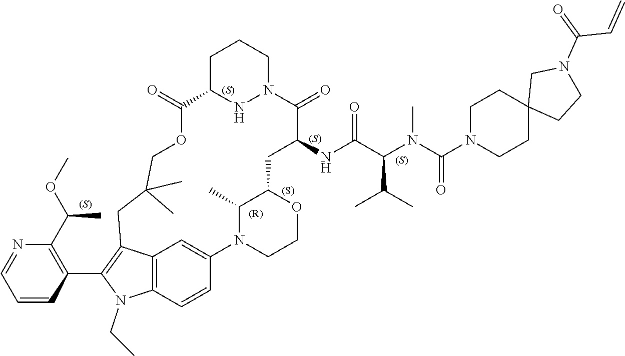 |
| A206 | 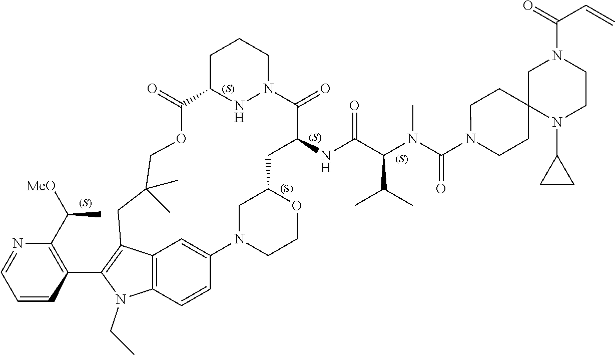 |
| A207 | 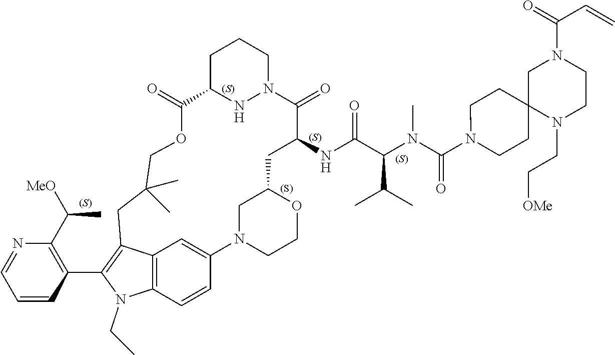 |
| A208 | 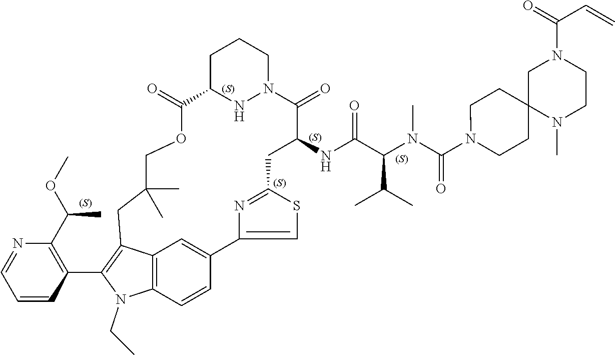 |
| A209 | 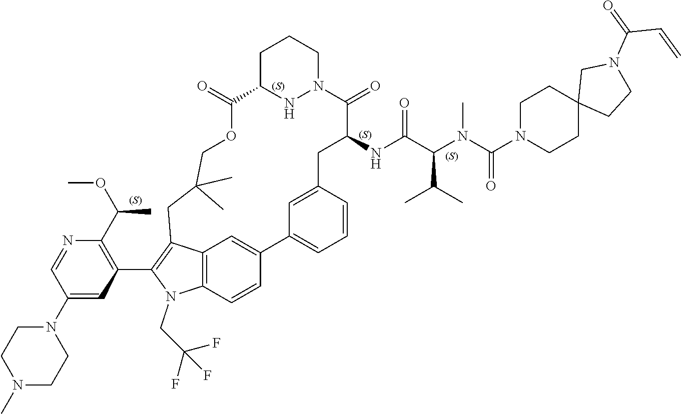 |
| A210 | 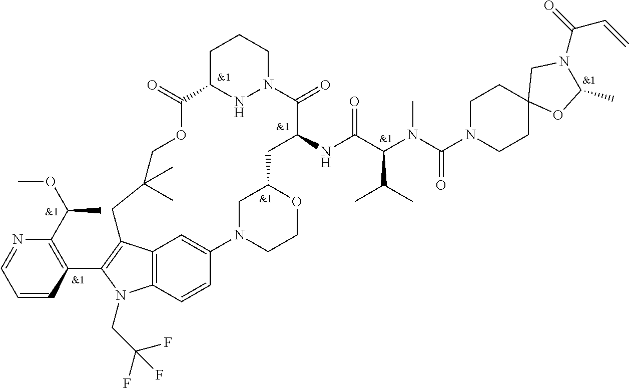 |
| A211 | 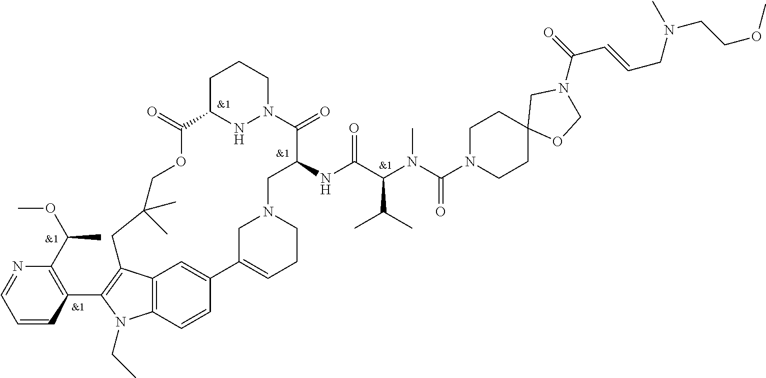 |
| A212 | 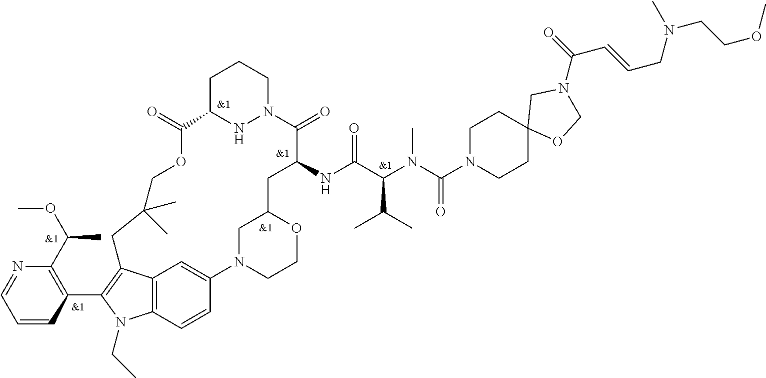 |
| A213 | 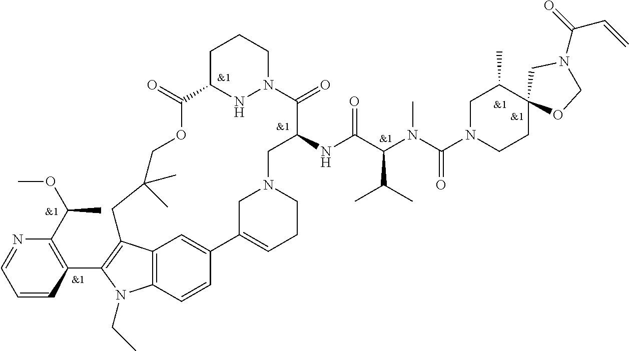 |
| A214 | 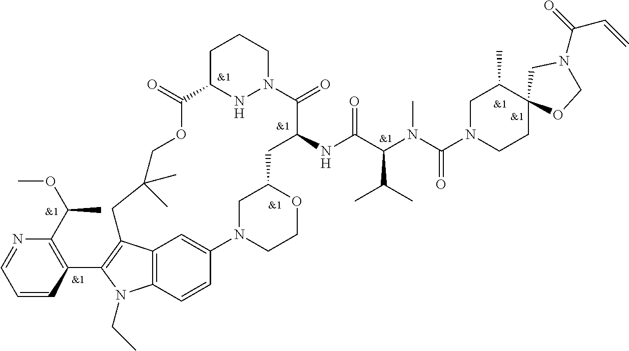 |
| A215 | 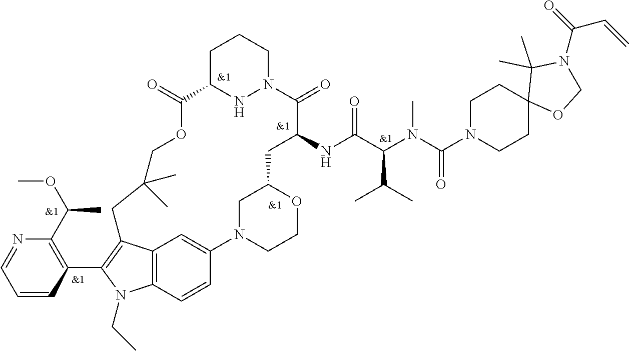 |
| A216 | 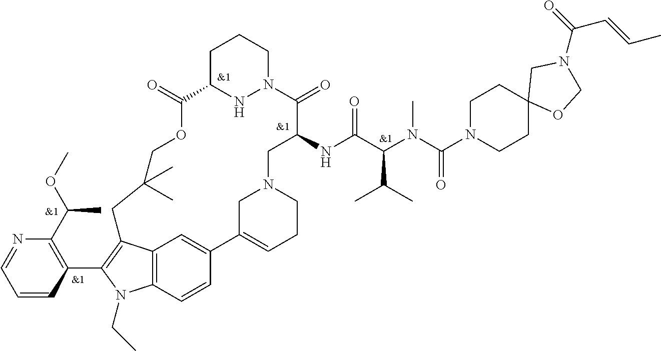 |
| A217 | 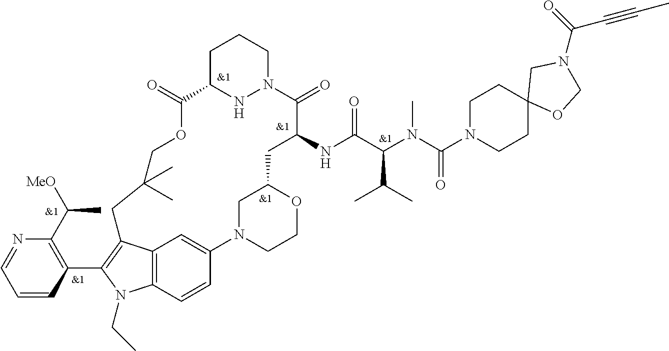 |
| A218 | 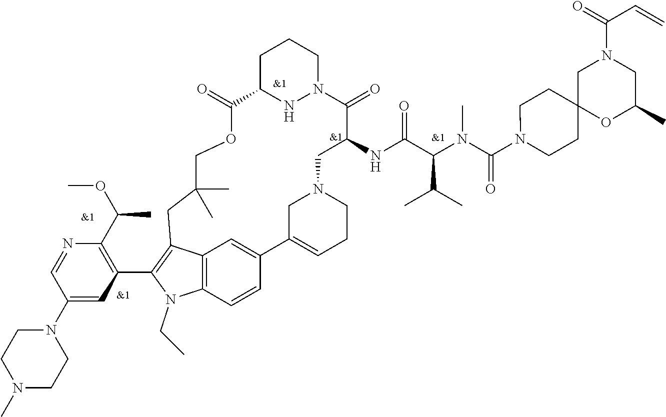 |
| A219 | 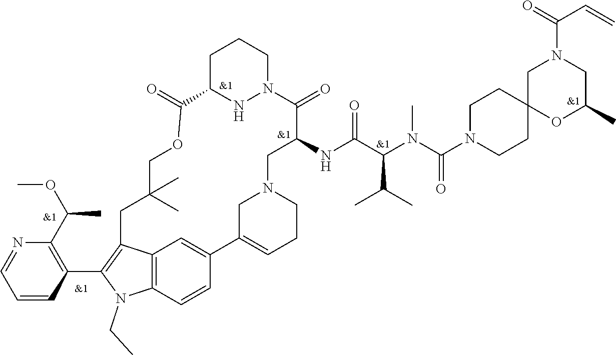 |
| A220 | 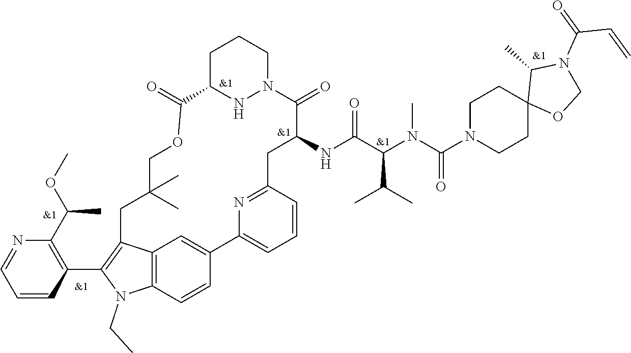 |
| A221 | 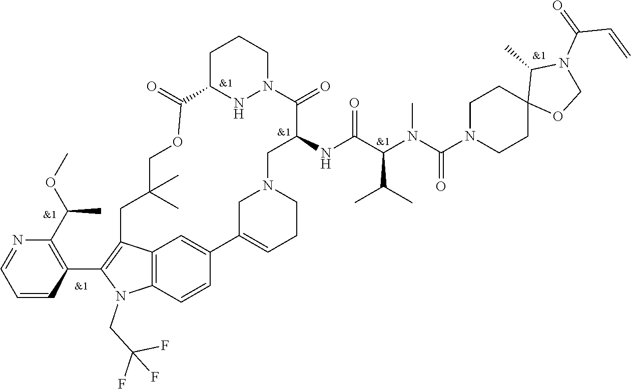 |
| A222 | 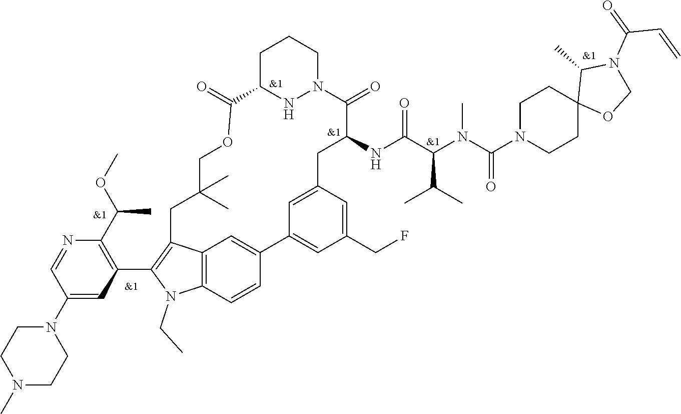 |
| A223 | 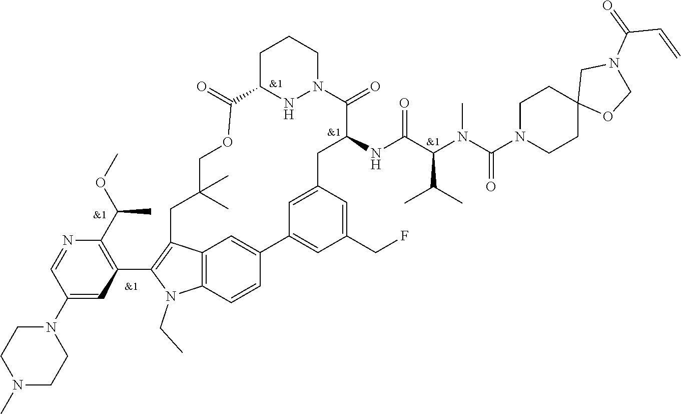 |
| A224 | 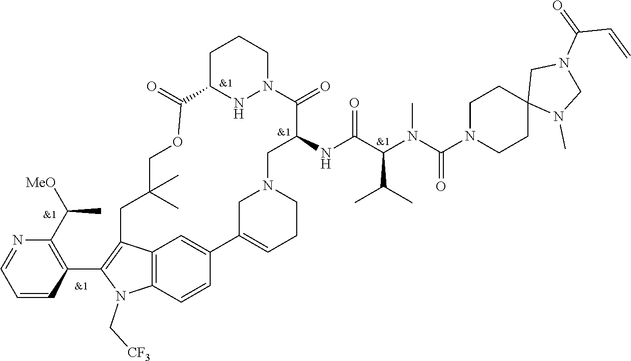 |
| A225 | 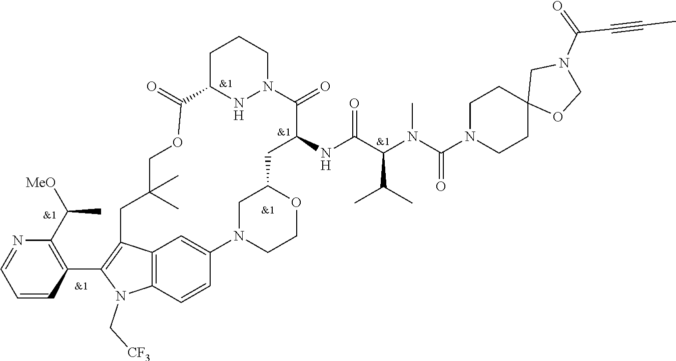 |
| A226 | 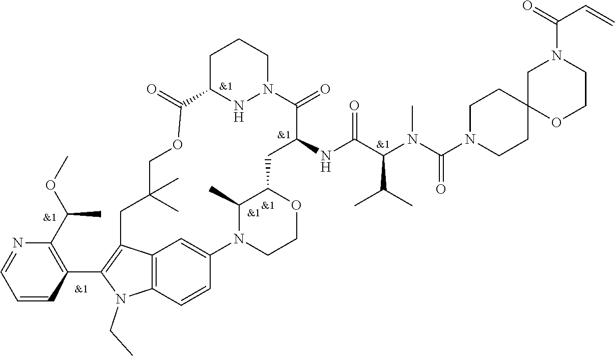 |
| A227 | 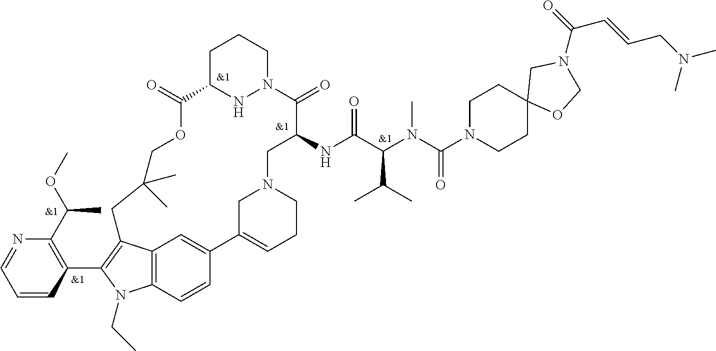 |
| A228 | 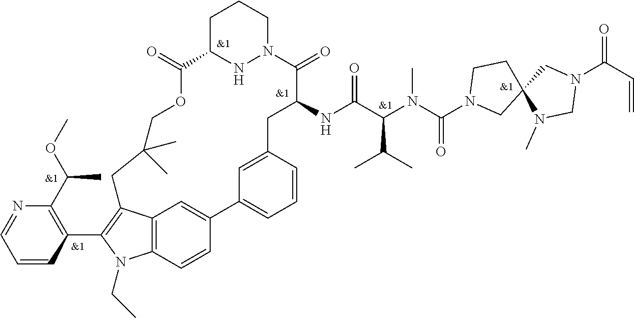 |
| A229 | 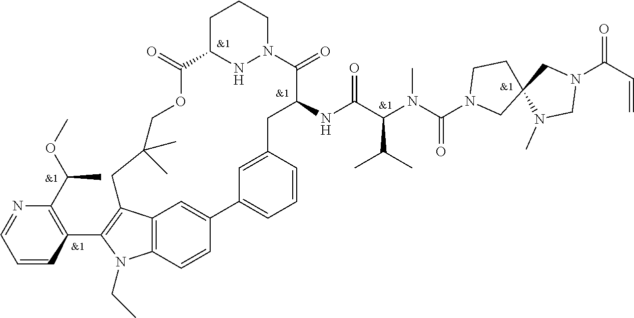 |
| A230 | 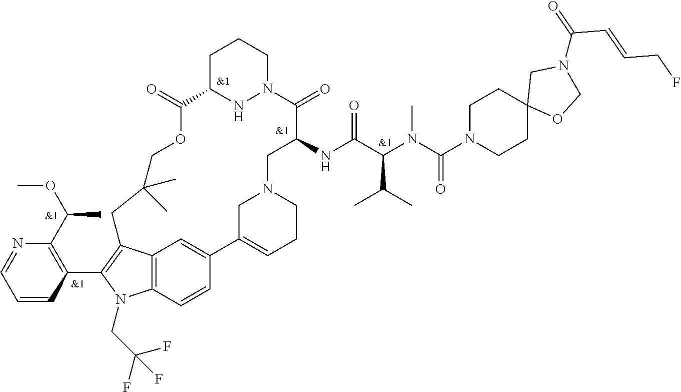 |
| A231 | 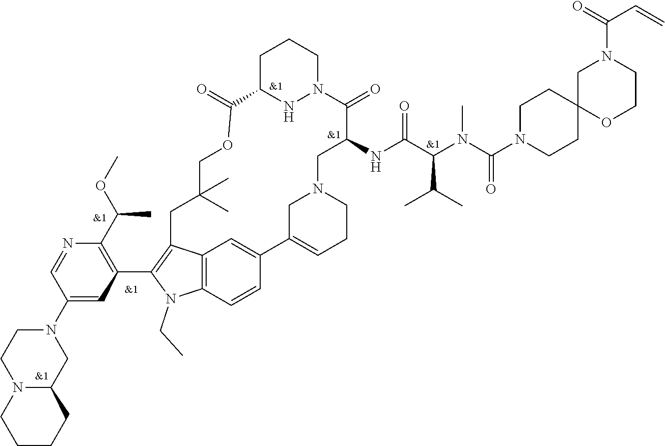 |
| A232 | 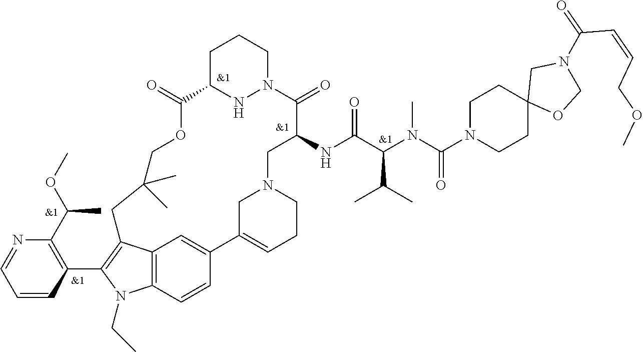 |
| A233 | 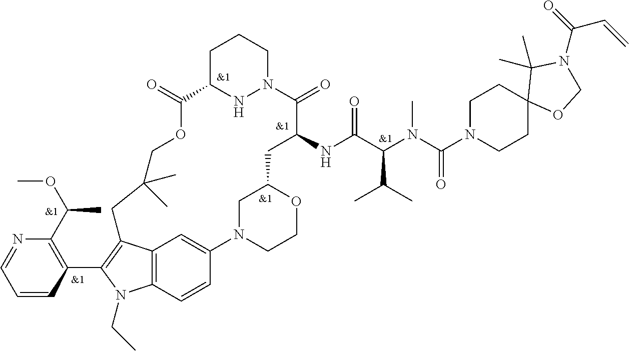 |
| A234 | 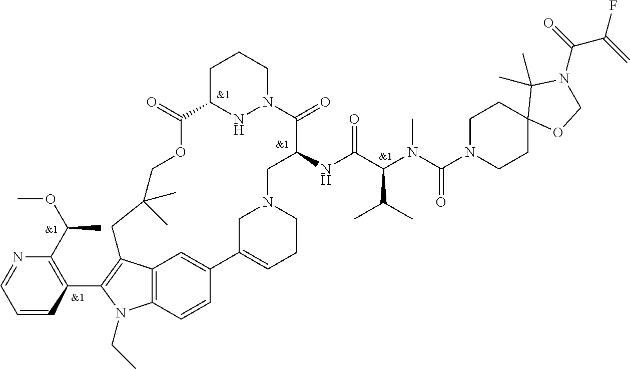 |
| A235 | 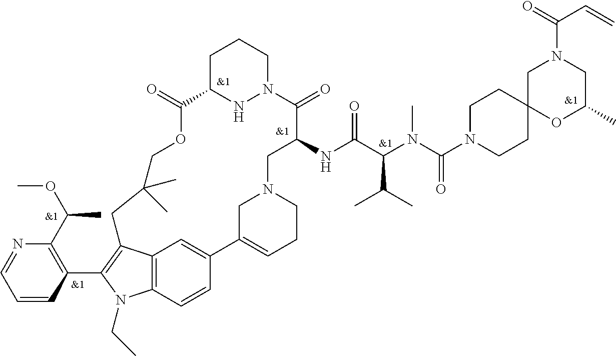 |
| A236 | 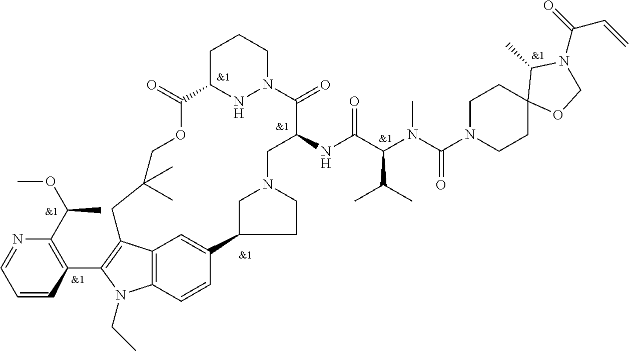 |
| A237 | 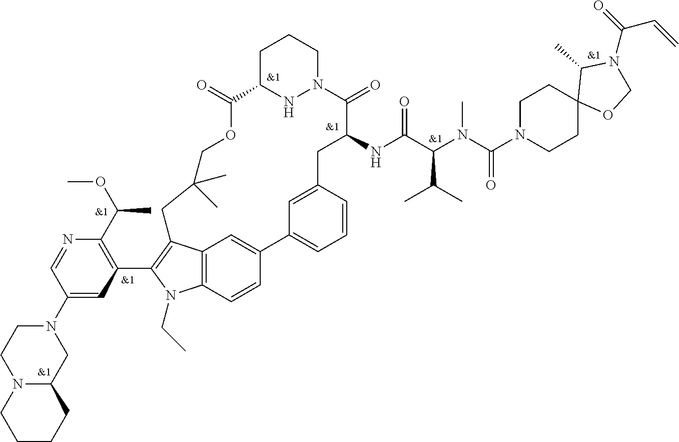 |
| A238 | 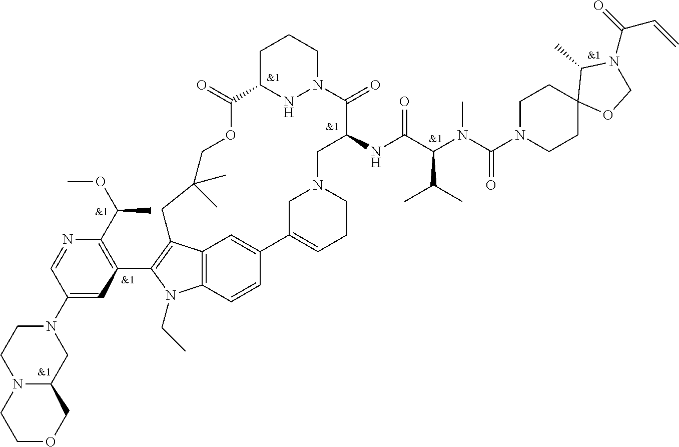 |
| A239 | 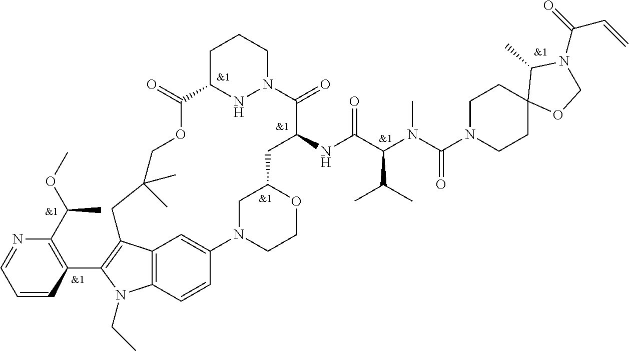 |
| A240 | 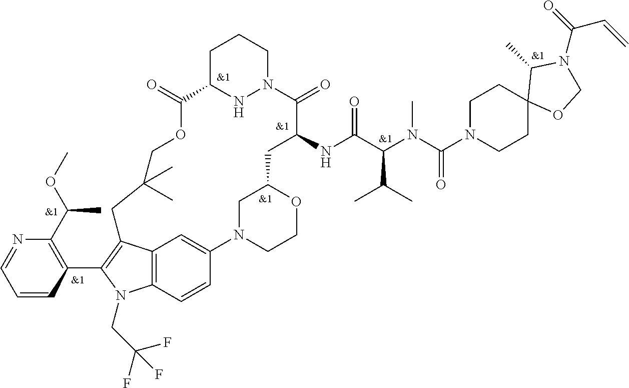 |
| A241 | 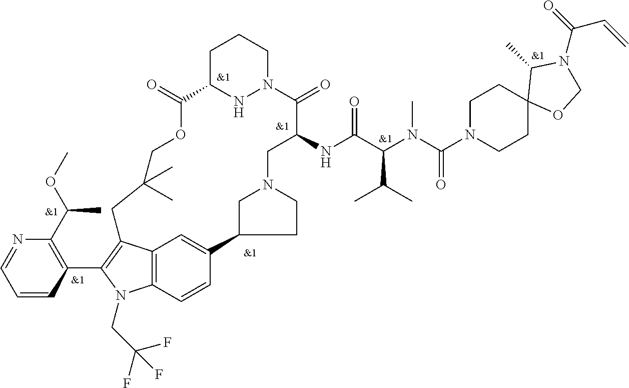 |
| A242 | 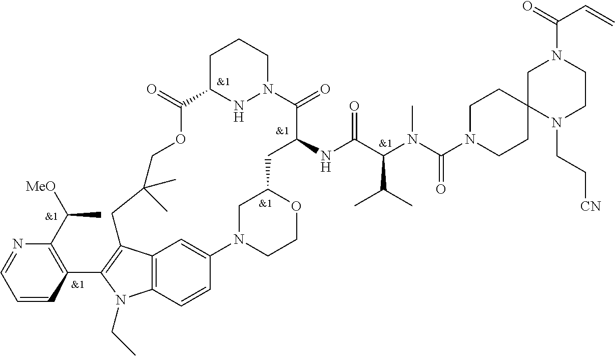 |
| A243 | 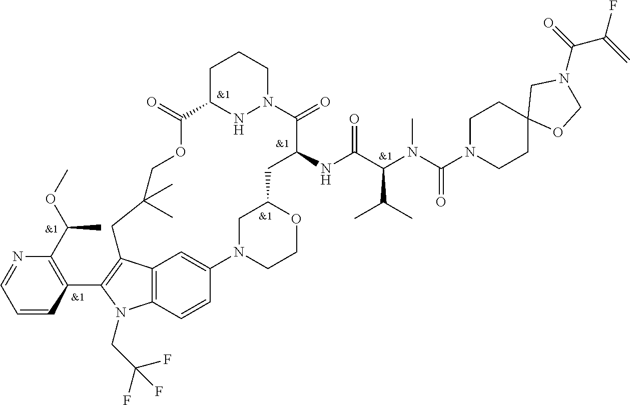 |
| A244 | 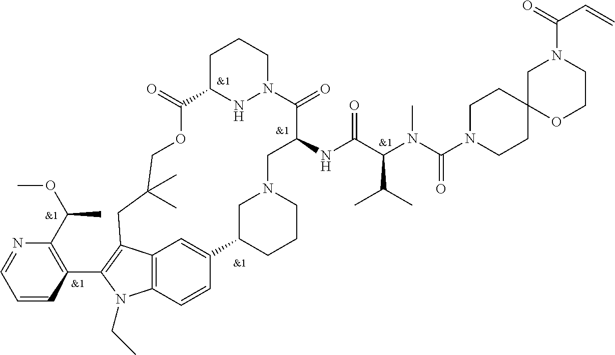 |
| A245 | 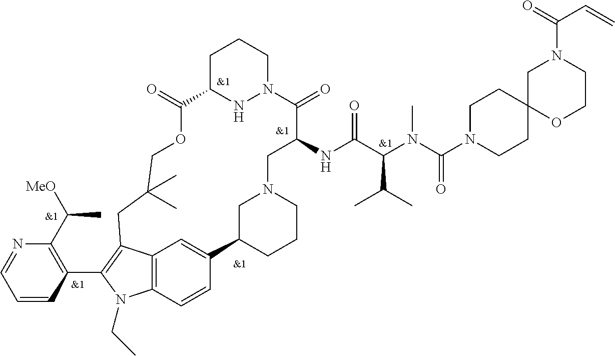 |
| A246 | 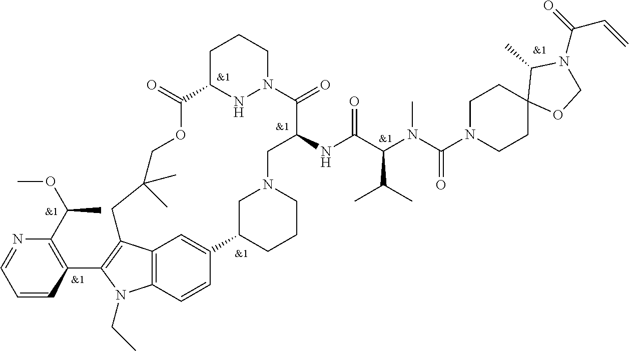 |
| A247 | 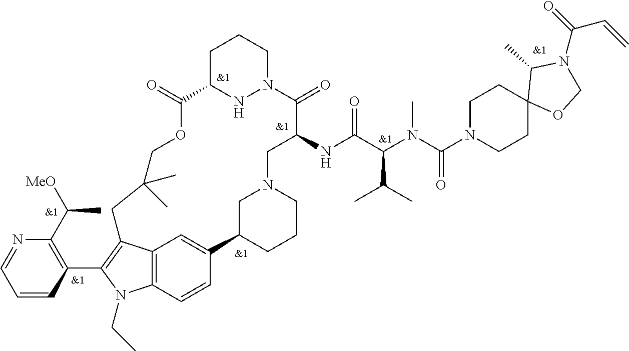 |
| A248 | 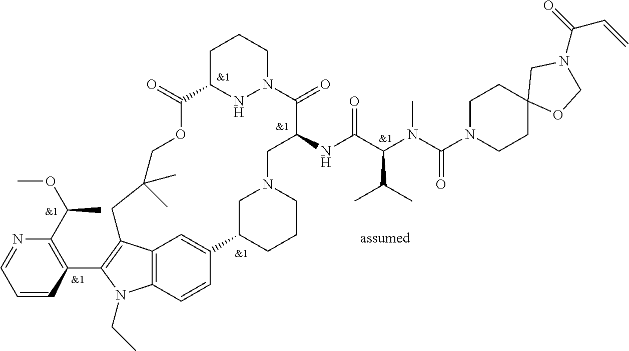 |
| A249 | 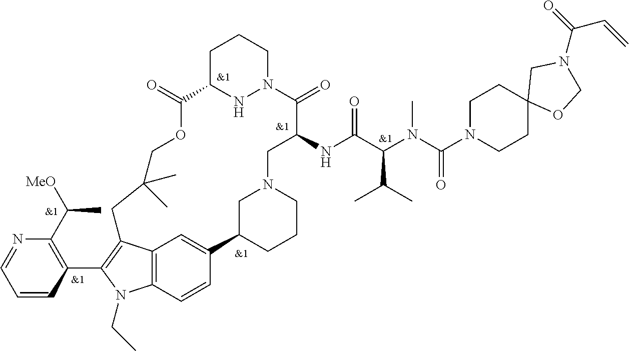 |
| A250 | 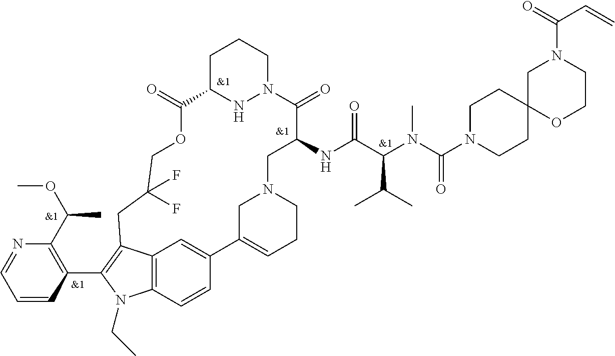 |
| A251 | 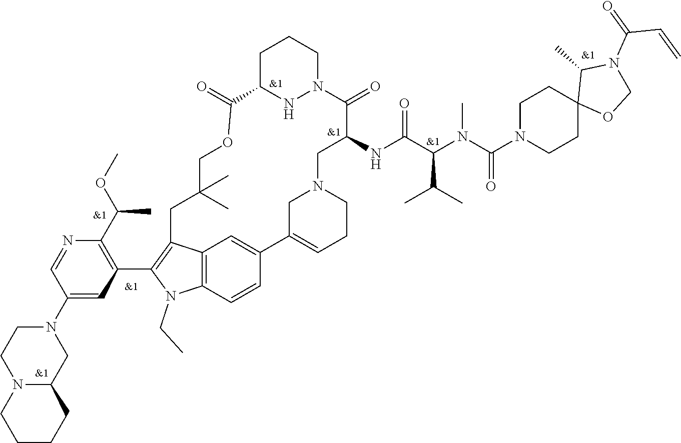 |
| A252 | 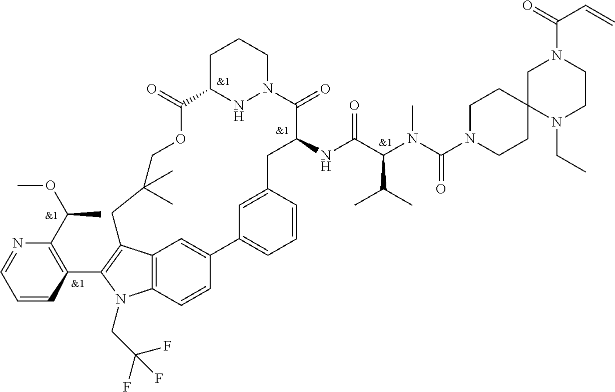 |
| A253 | 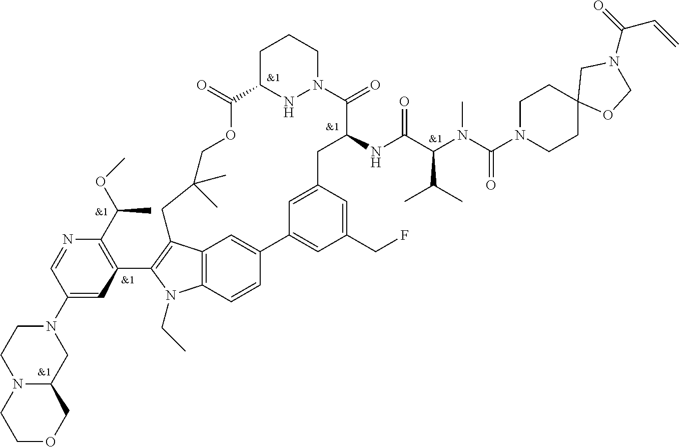 |
| A254 | 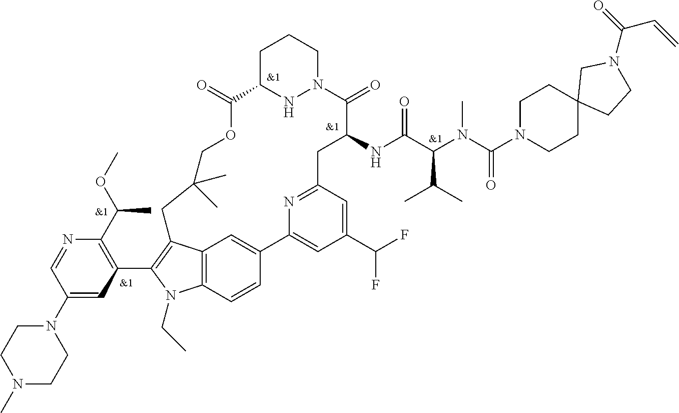 |
| A255 | 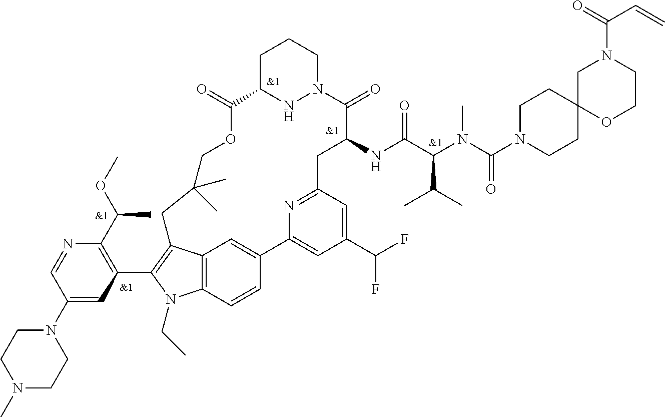 |
| A256 | 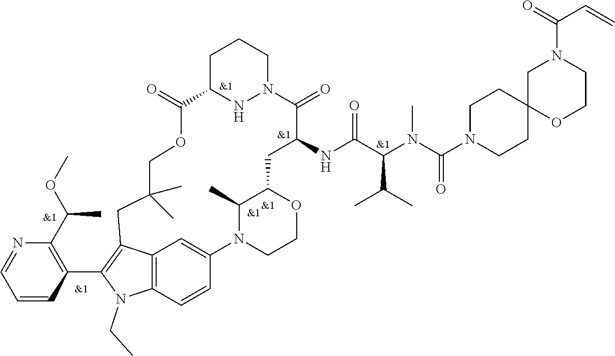 |
| A257 | 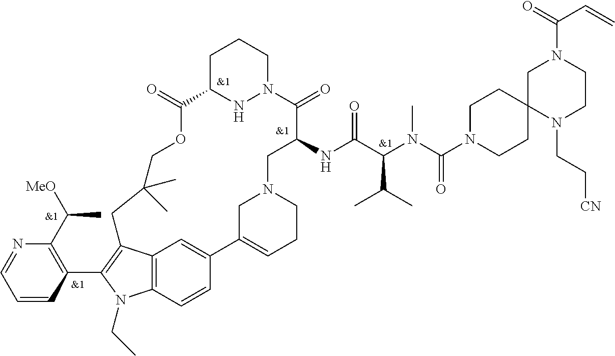 |
| A258 | 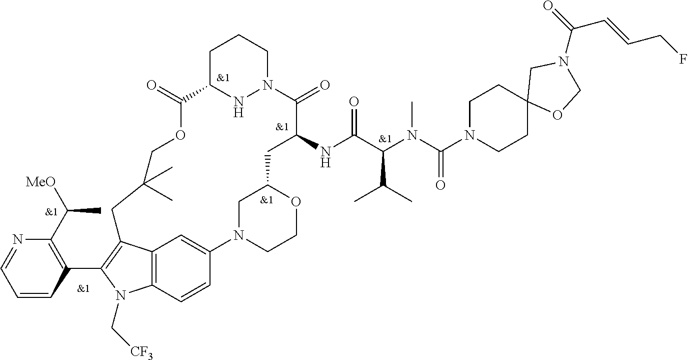 |
| A259 | 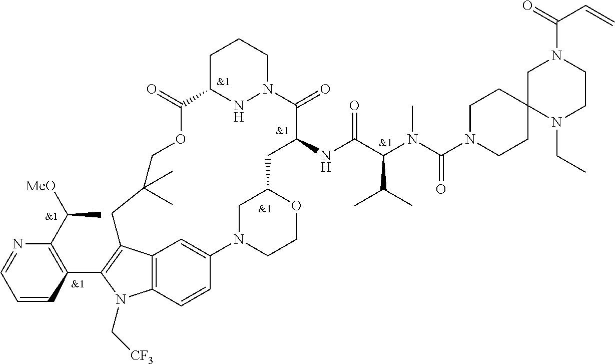 |
| A260 | 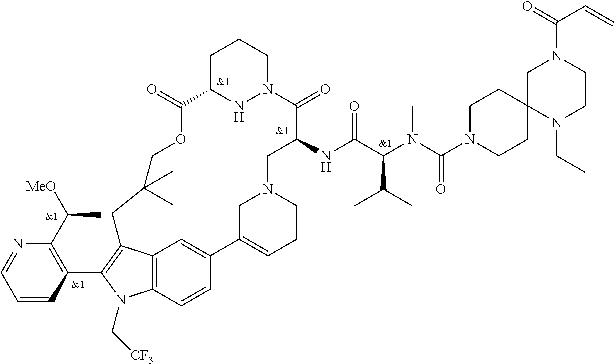 |
| A261 | 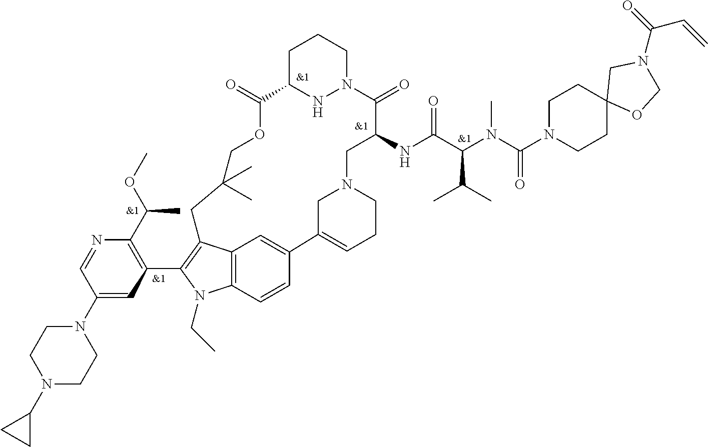 |
| A262 | 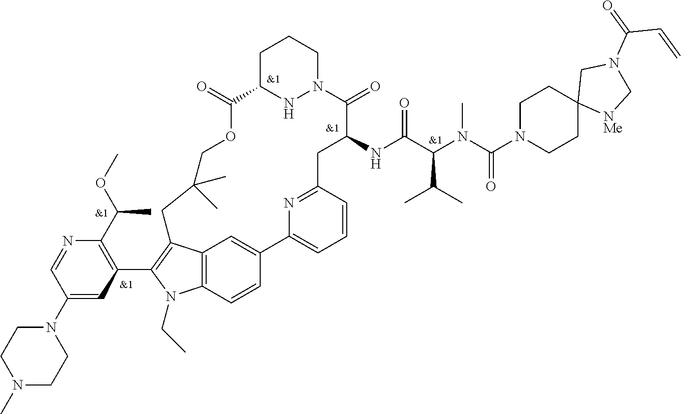 |
| A263 | 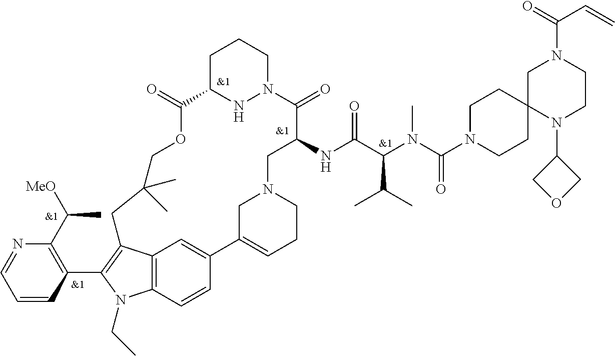 |
| A264 | 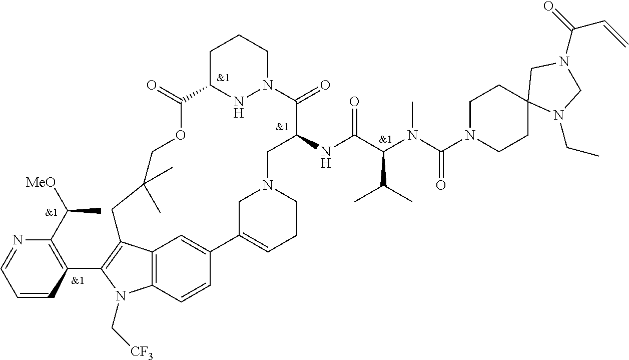 |
| A265 | 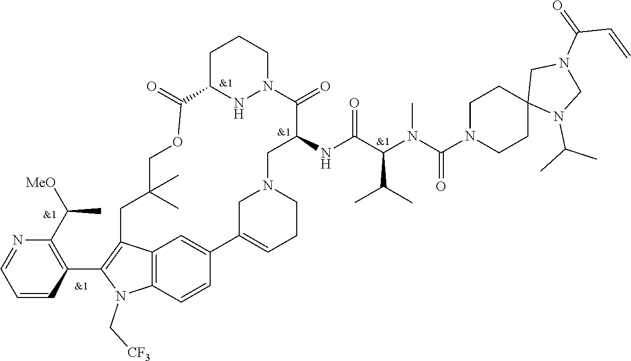 |
| A266 | 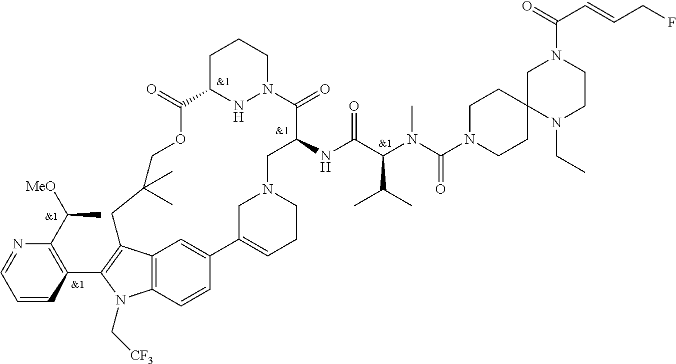 |
| A267 | 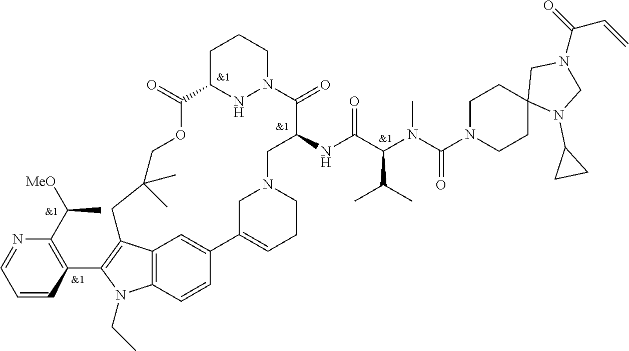 |
| A268 | 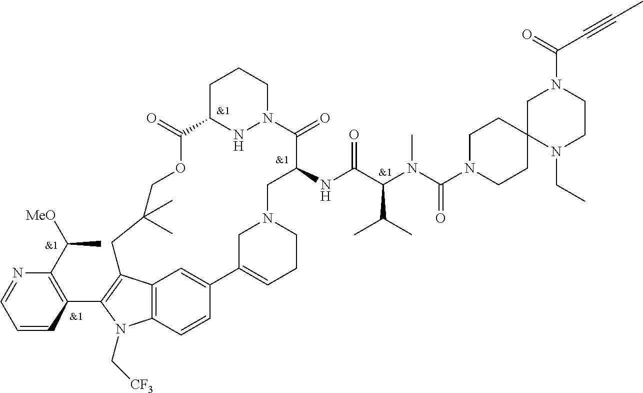 |
| A269 | 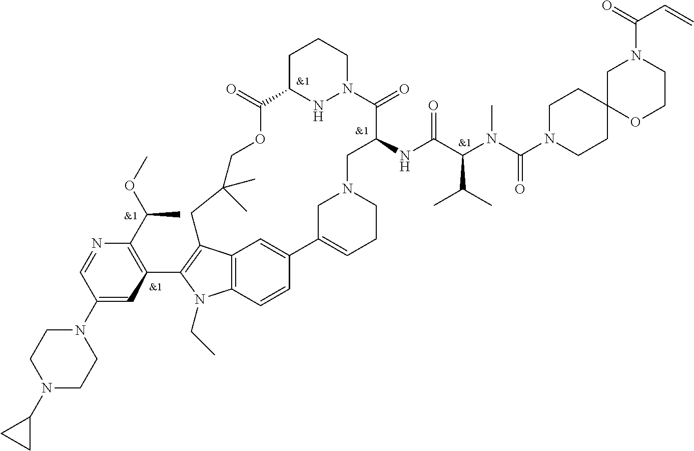 |
| A270 | 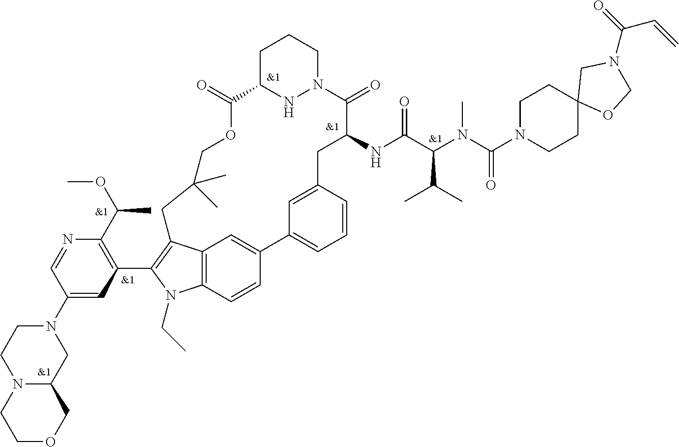 |
| A271 | 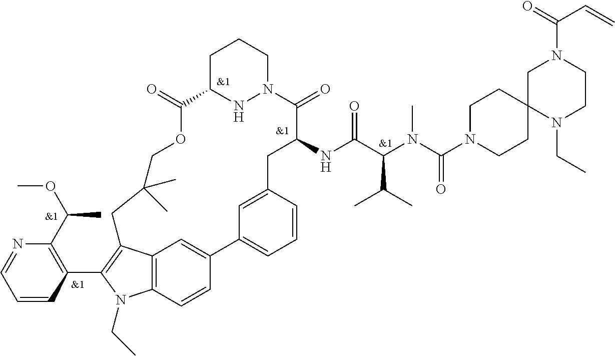 |
| A272 | 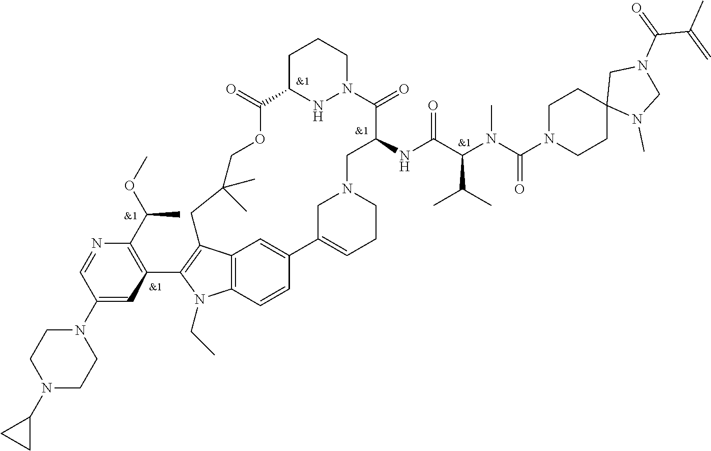 |
| A273 | 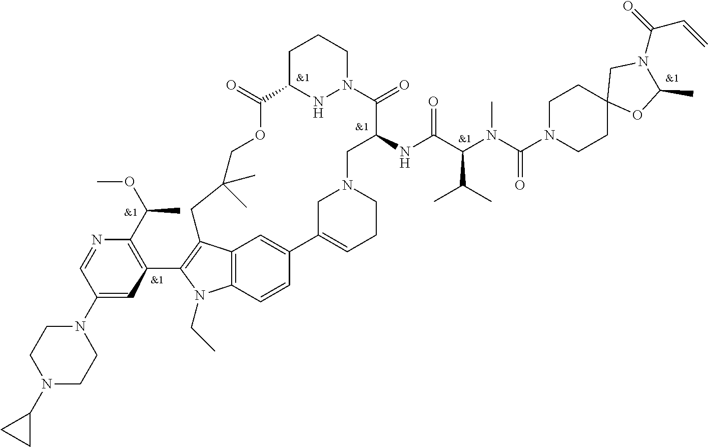 |
| A274 | 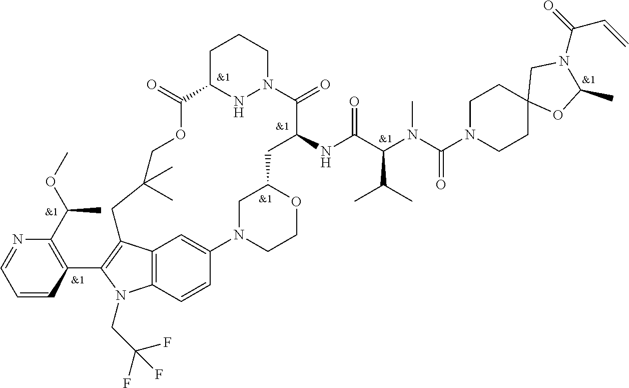 |
| A275 | 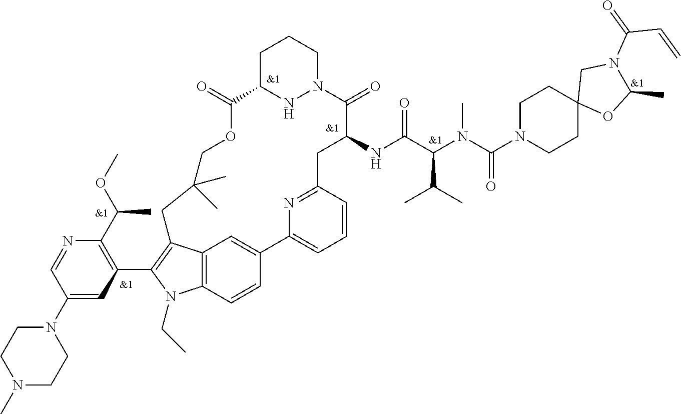 |
| A276 | 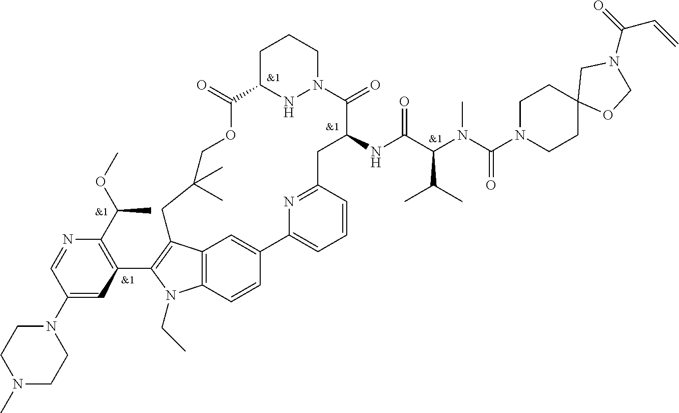 |
| A277 | 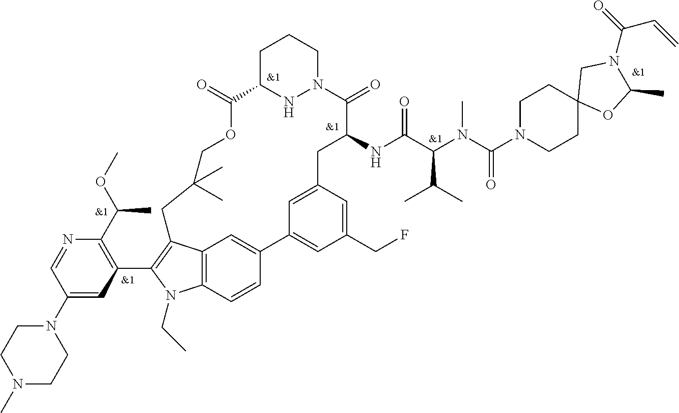 |
| A278 | 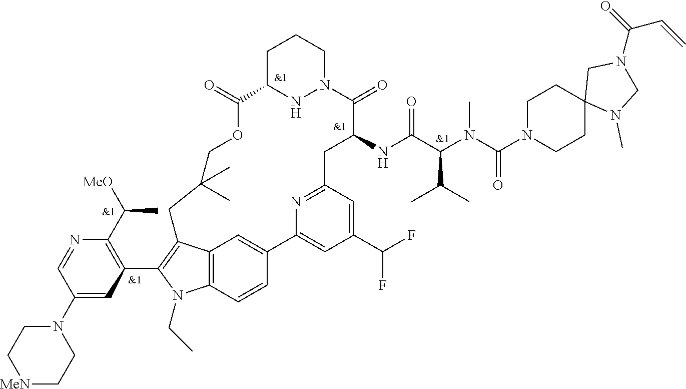 |
| A279 | 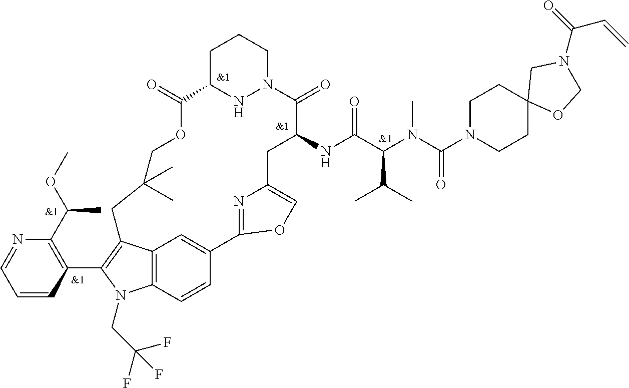 |
| A280 | 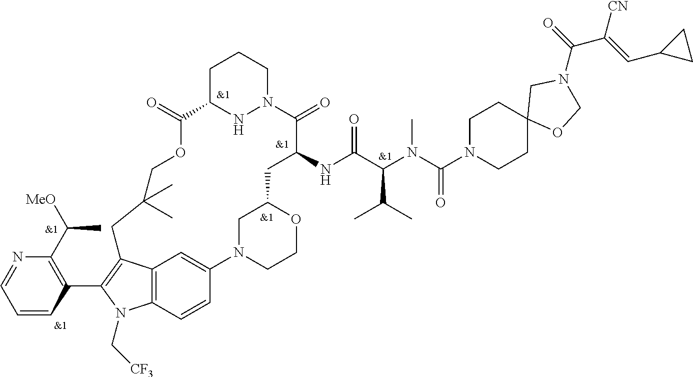 |
| A281 | 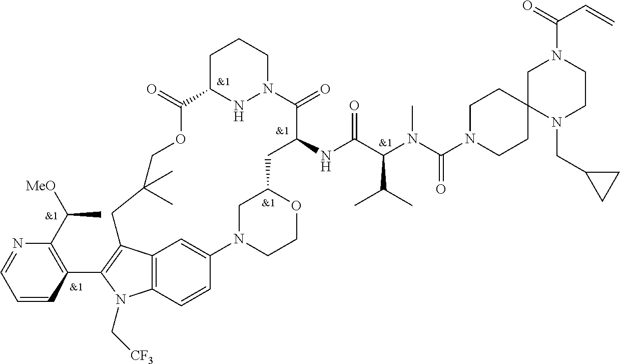 |
| A282 | 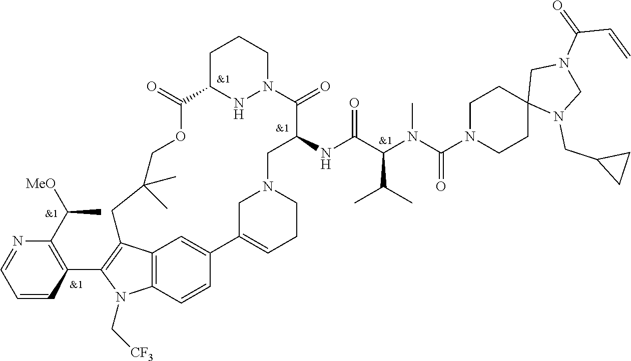 |
| A283 | 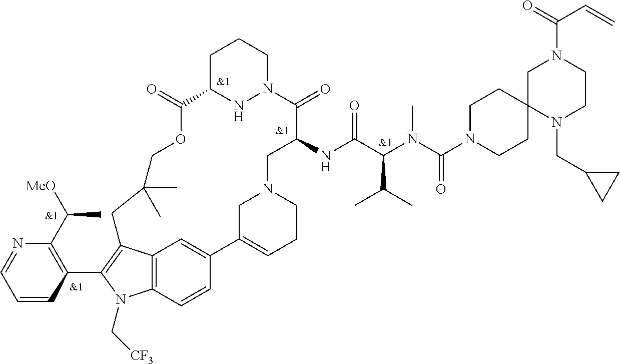 |
| A284 | 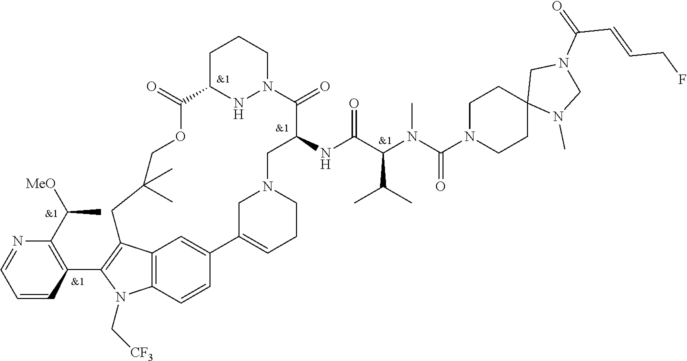 |
| A285 | 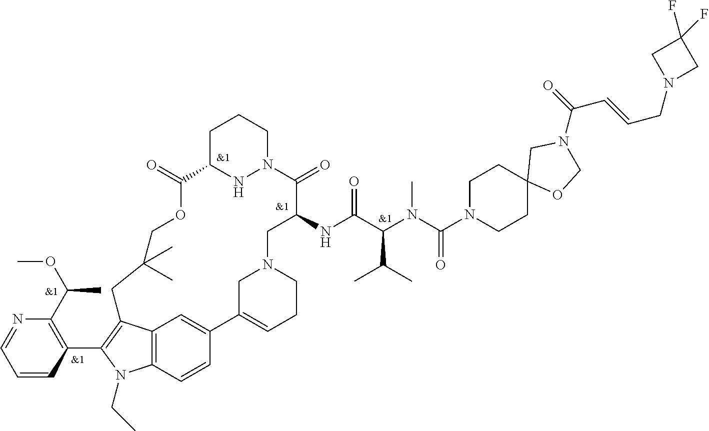 |
| A286 | 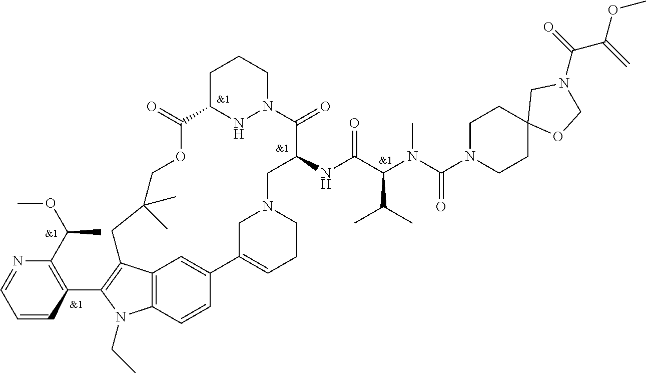 |
| A287 | 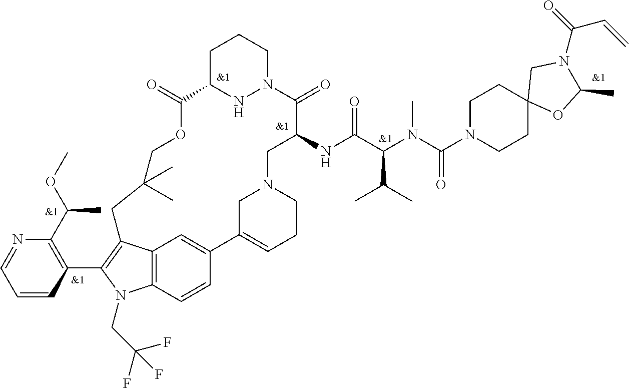 |
| A288 | 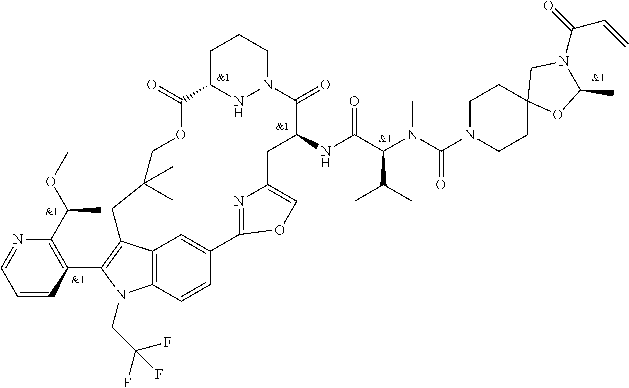 |
| A289 | 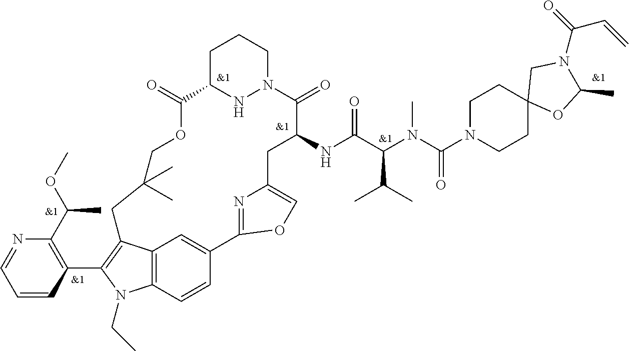 |
| A290 | 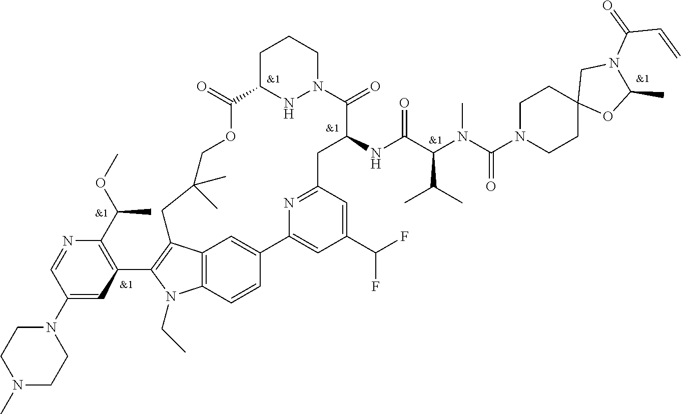 |
| A291 | 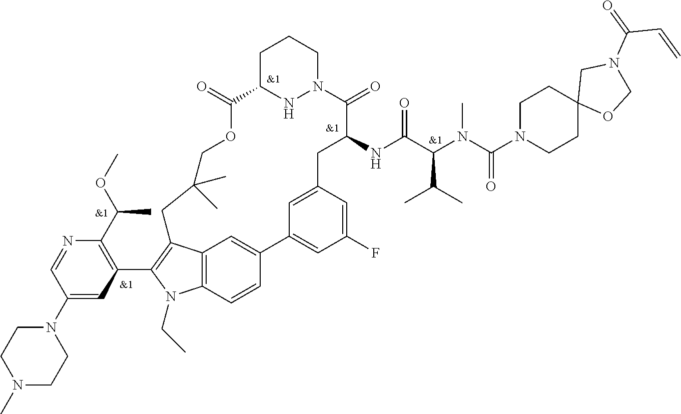 |
| A292 | 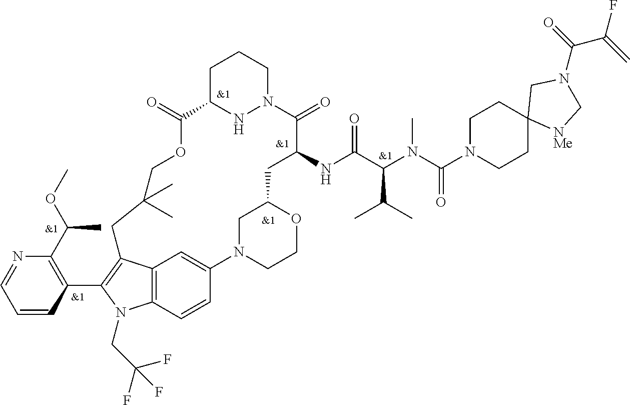 |
| A293 | 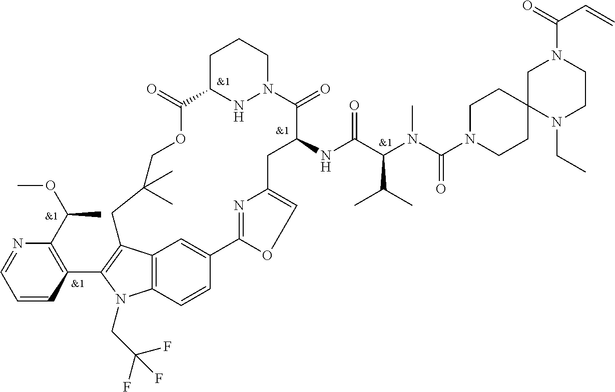 |
| A294 | 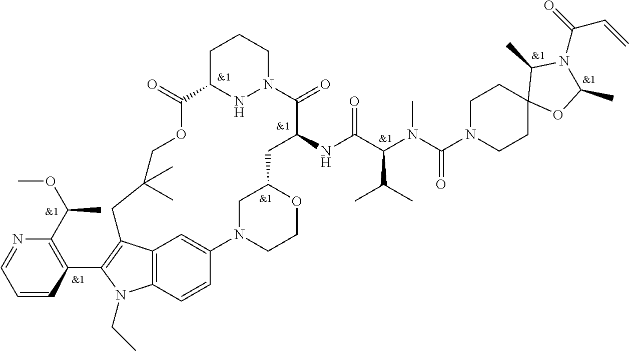 |
| A295 | 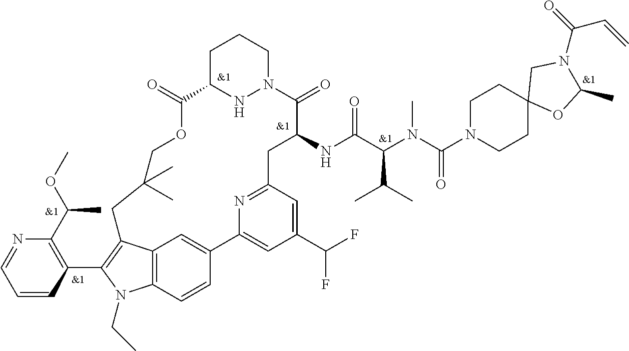 |
| A296 | 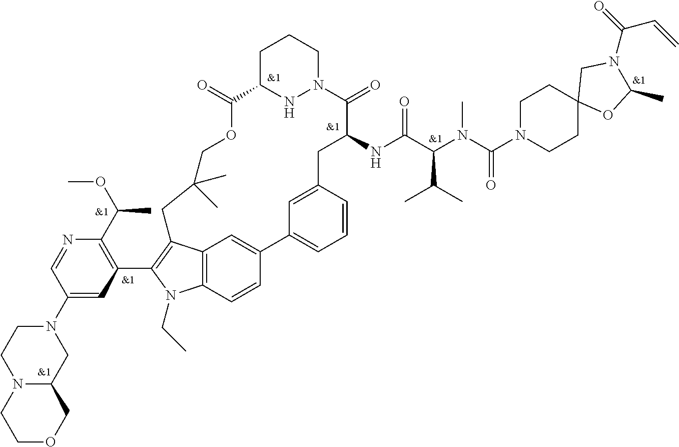 |
| A297 | 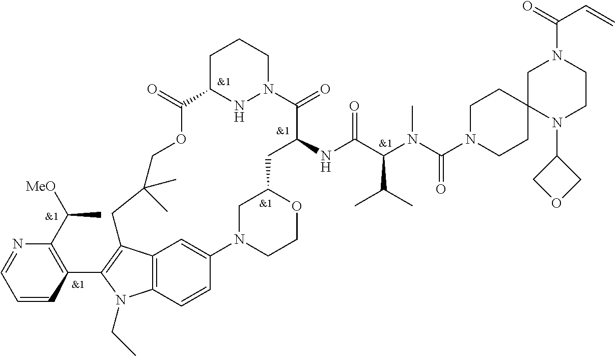 |
| A298 | 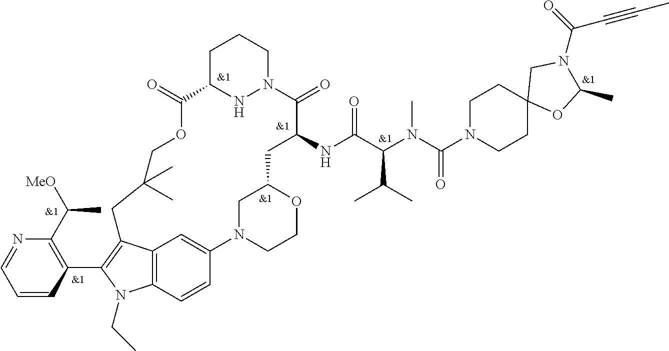 |
| A299 | 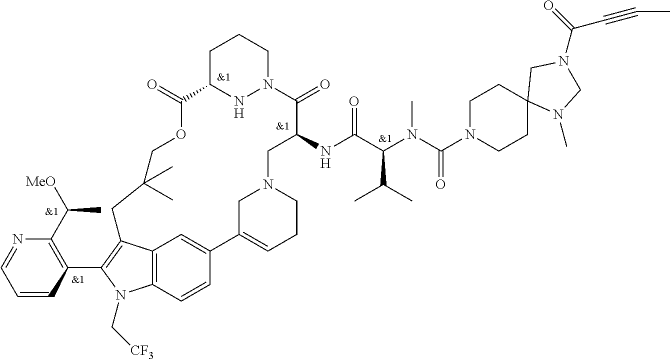 |
| A300 | 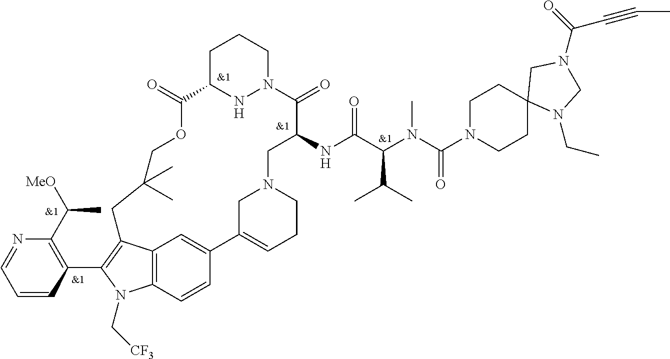 |
| A301 | 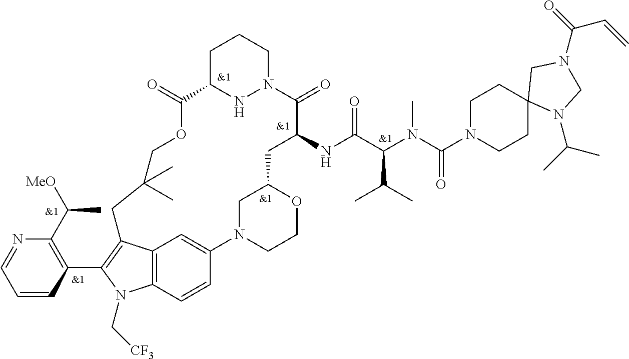 |
| A302 | 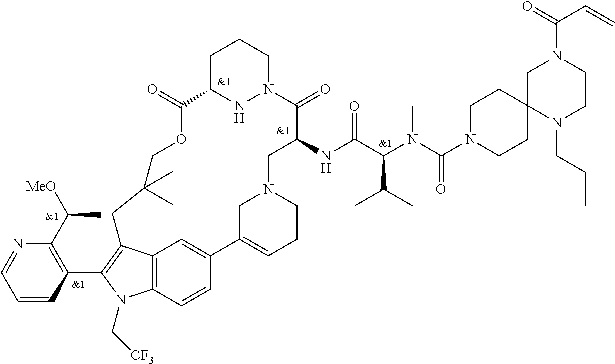 |
| A303 | 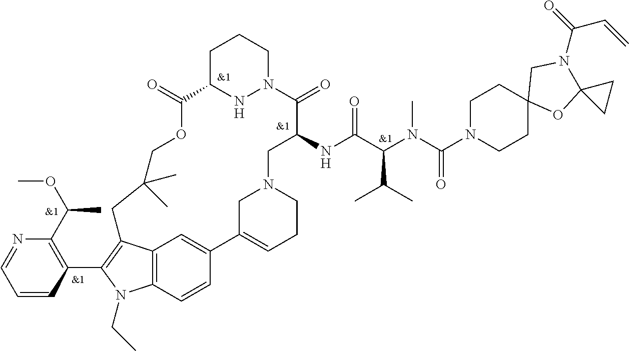 |
| A304 | 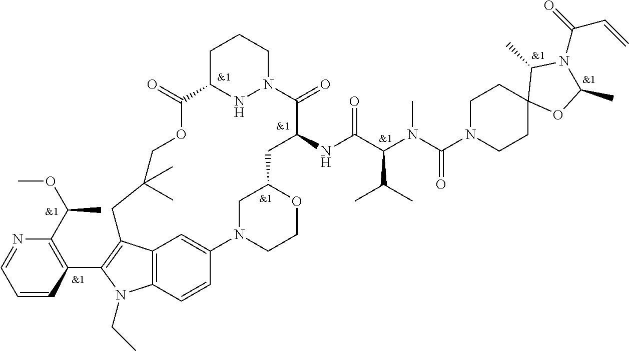 |
| A305 | 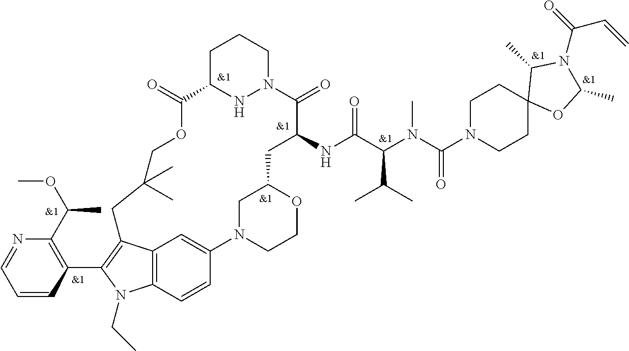 |
| A306 | 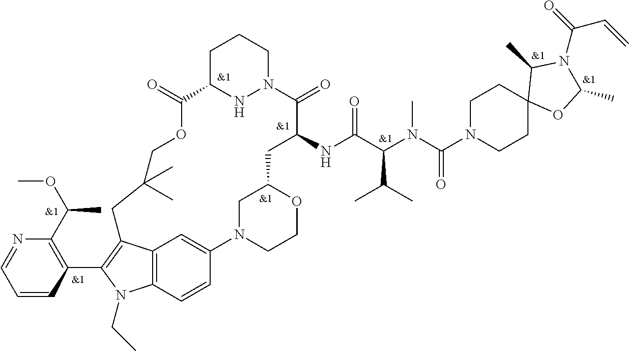 |
| A307 | 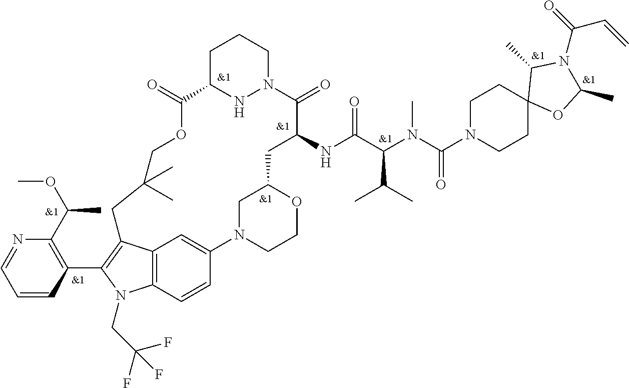 |
| A308 | 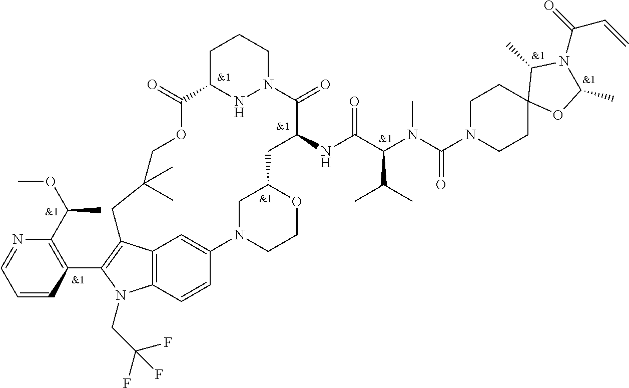 |
| A309 | 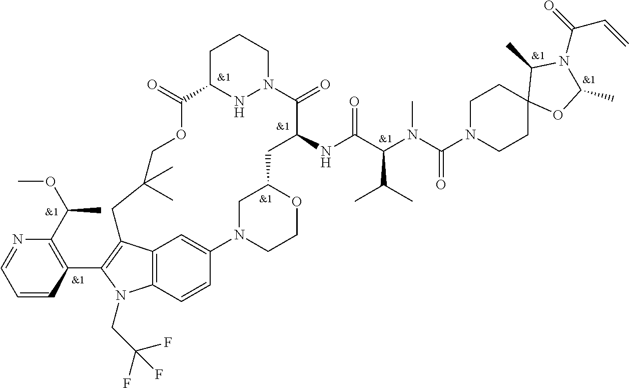 |
| A310 | 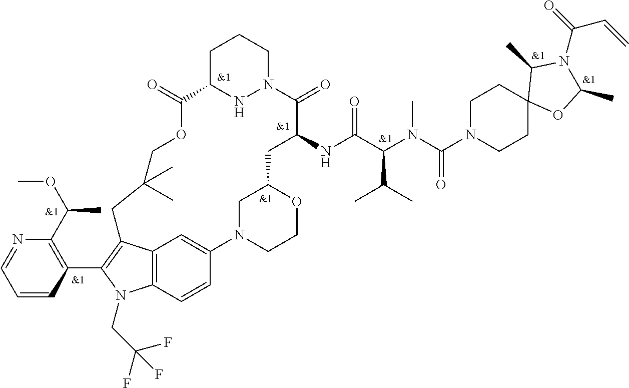 |
| A311 | 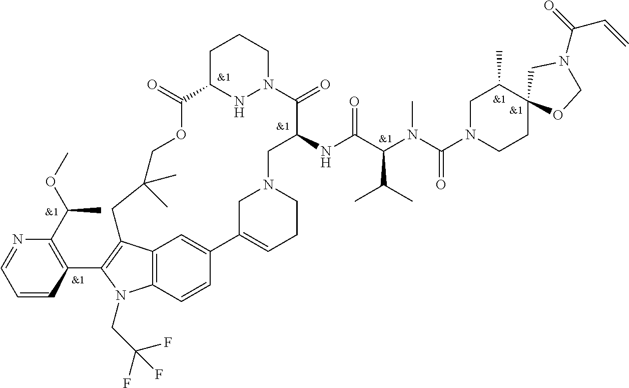 |
| A312 | 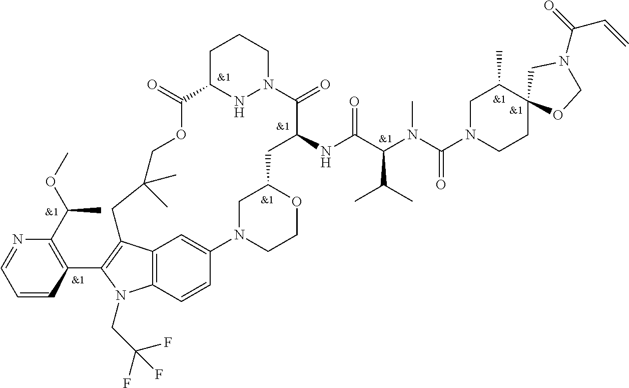 |
| A313 | 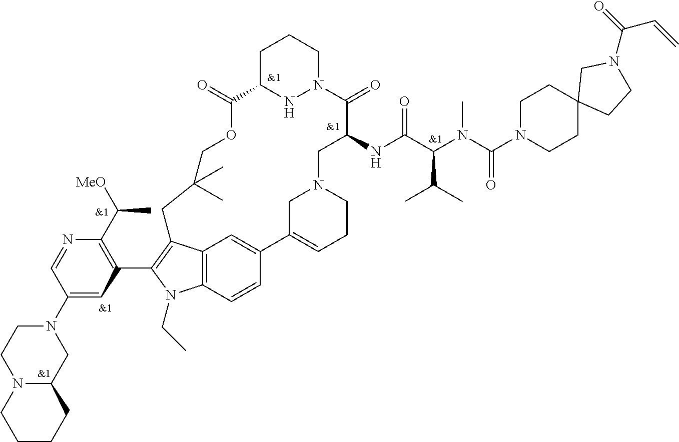 |
| A314 | 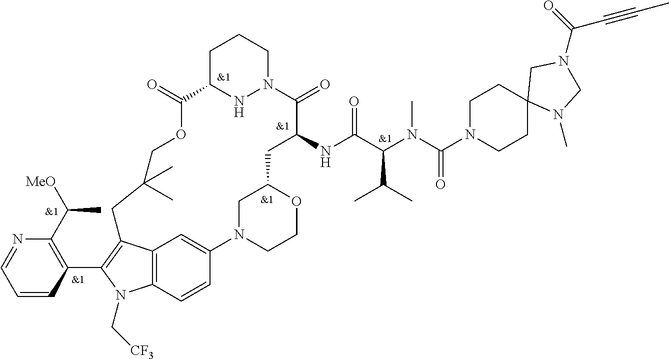 |
| A315 | 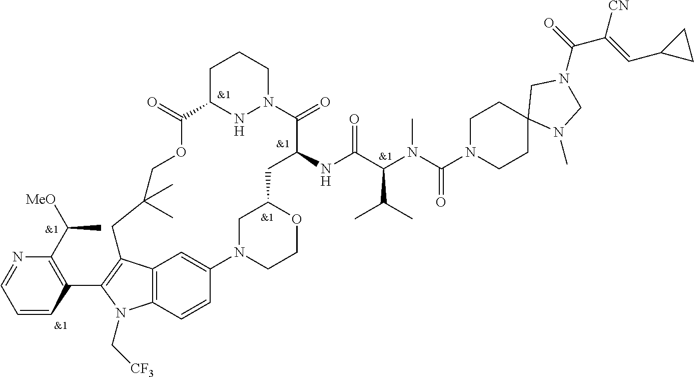 |
| A316 | 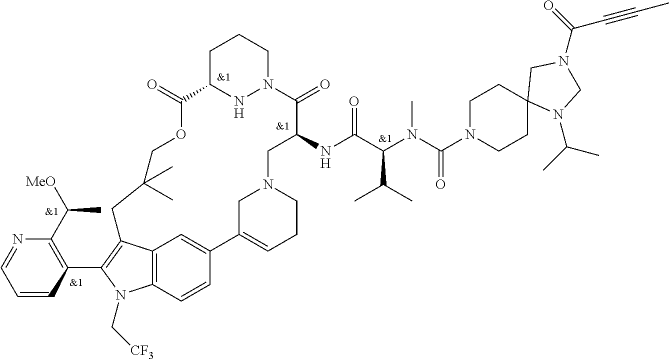 |
| A317 | 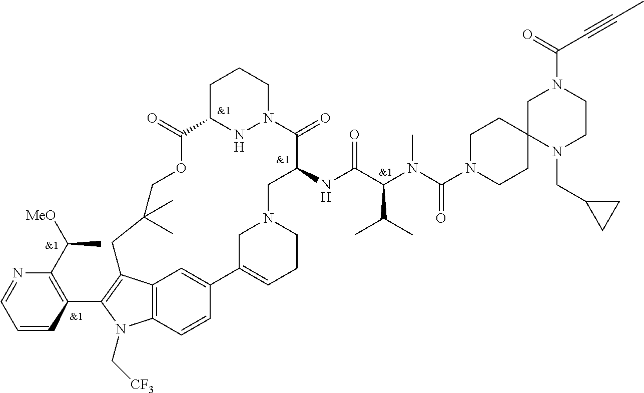 |
| A318 | 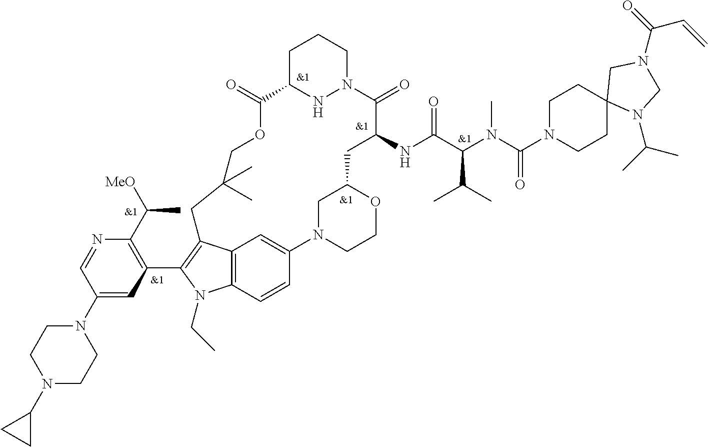 |
| A319 | 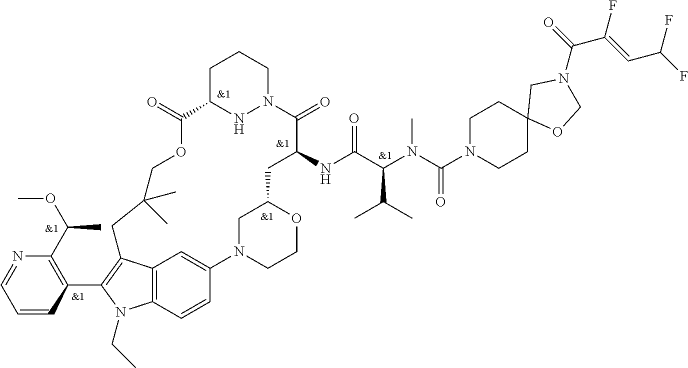 |
| A320 | 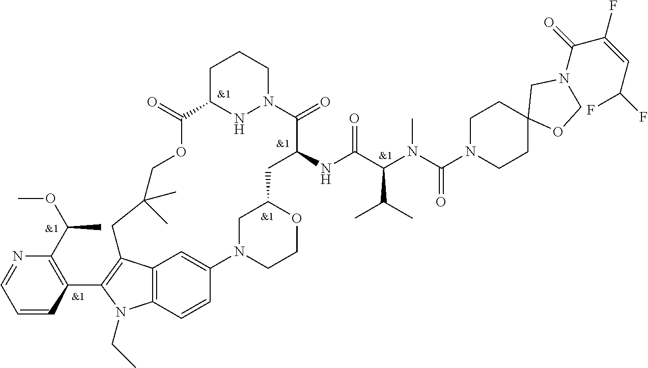 |
| A321 | 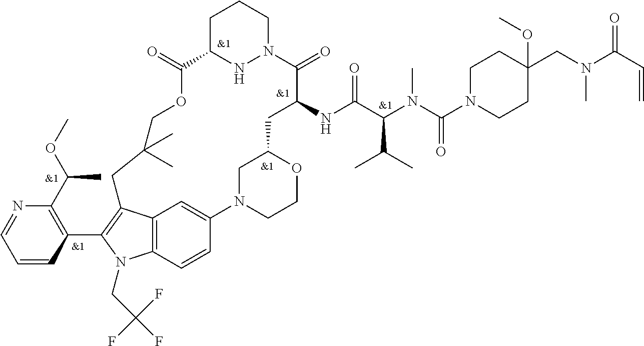 |
| A322 | 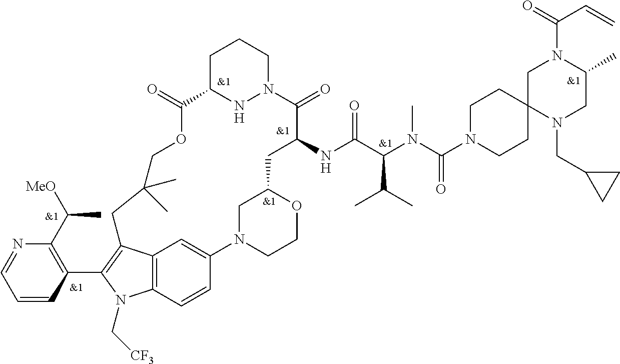 |
| A323 | 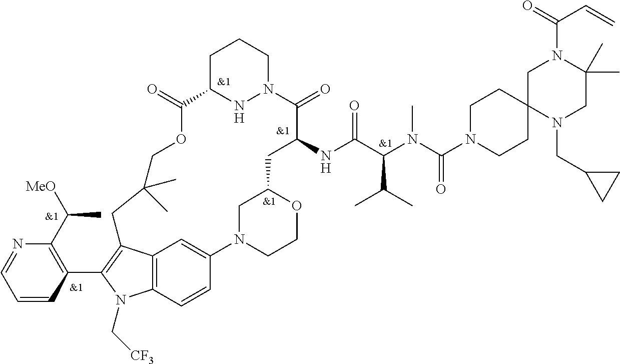 |
| A324 | 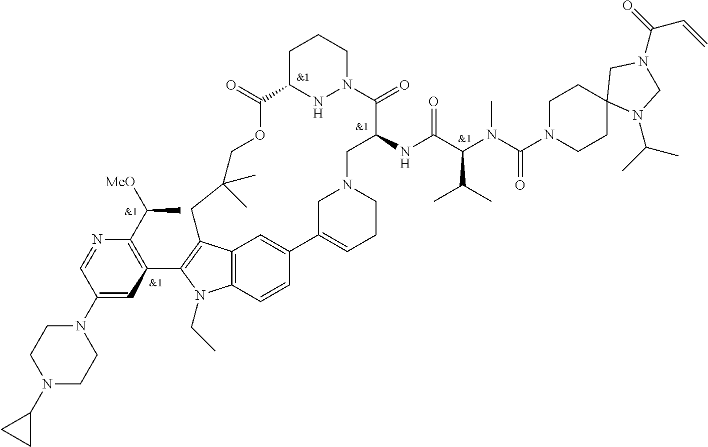 |
| A325 | 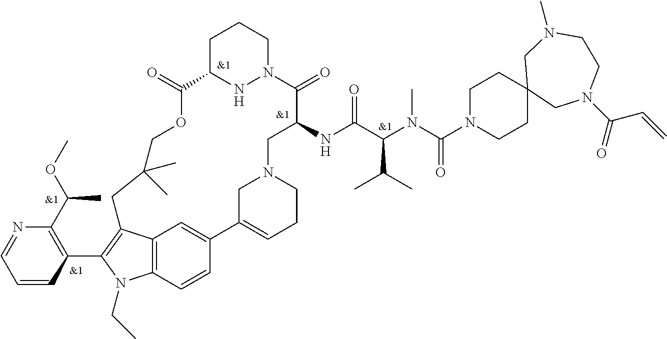 |
| A326 | 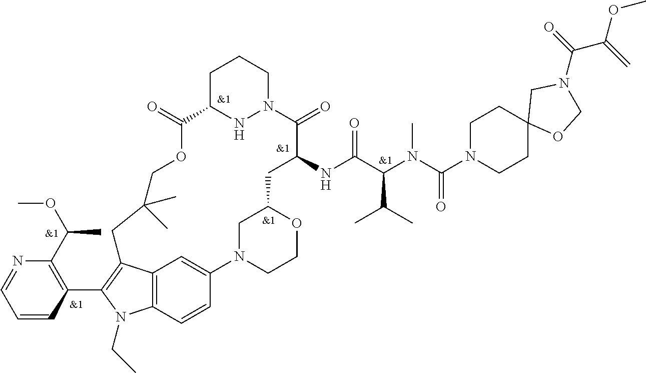 |
| A327 | 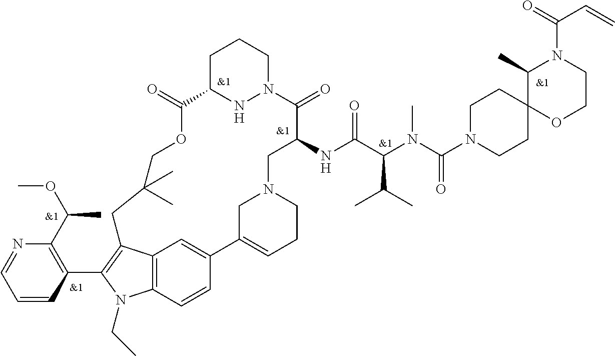 |
| A328 | 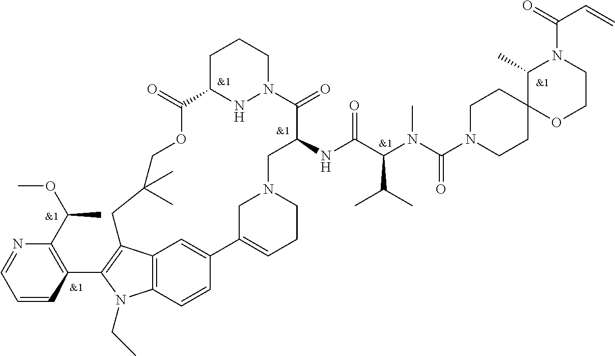 |
| A329 | 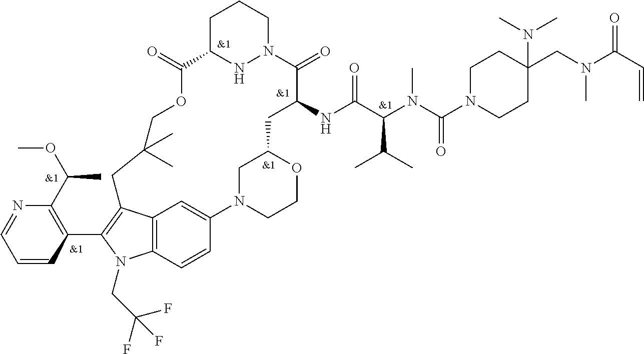 |
| A330 | 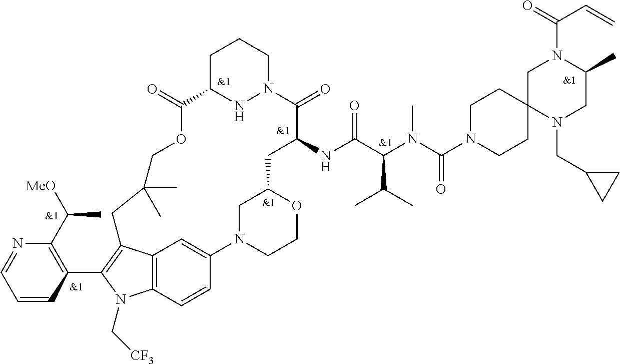 |
| A331 | 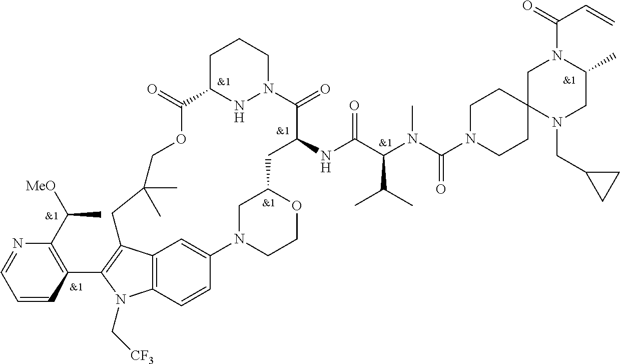 |
| A332 | 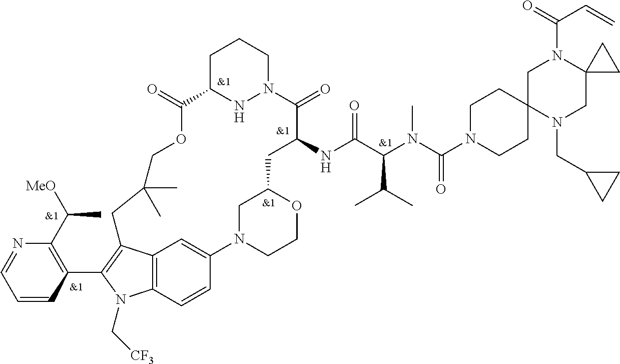 |
| A333 | 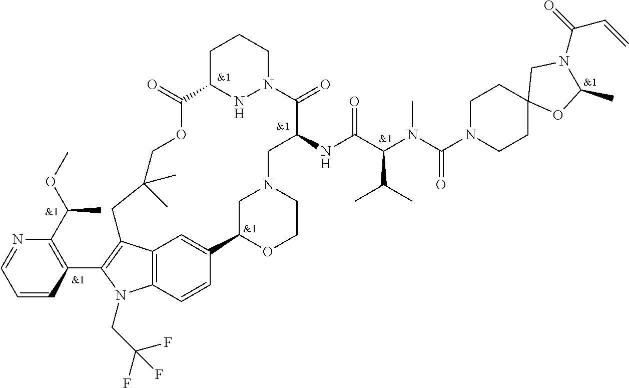 |
| A334 | 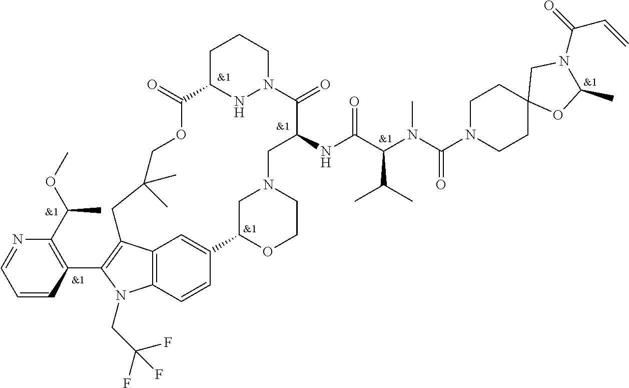 |
| A335 | 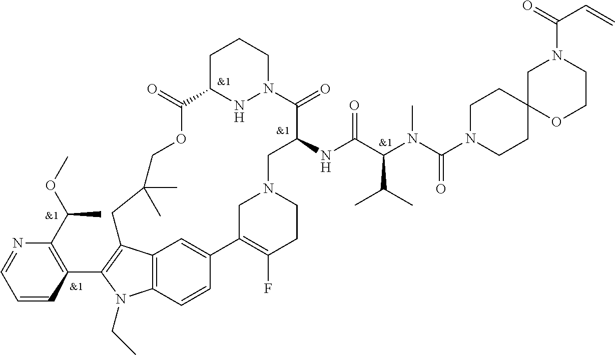 |
| A336 | 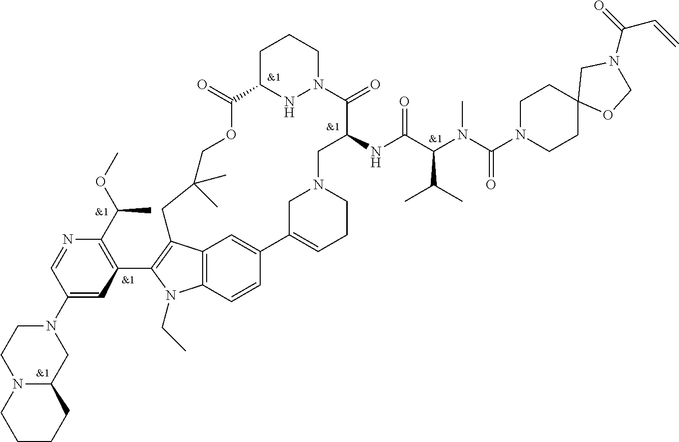 |
| A337 | 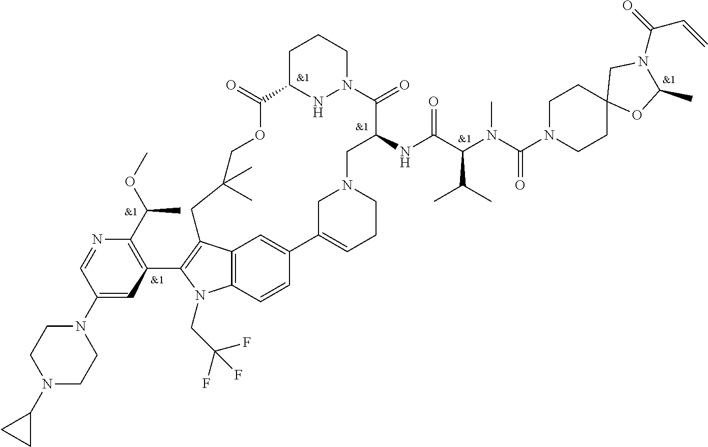 |
| A338 | 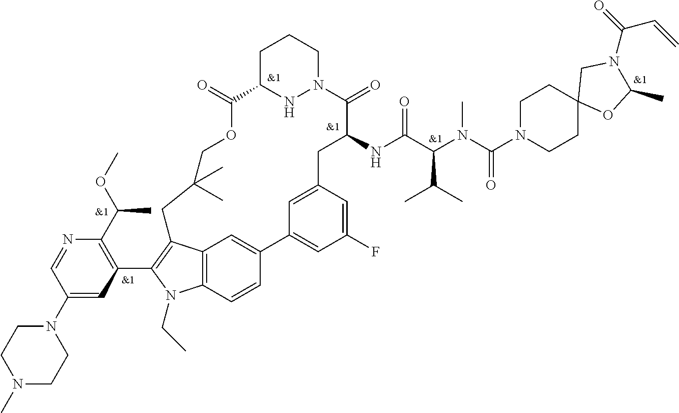 |
| A339 | 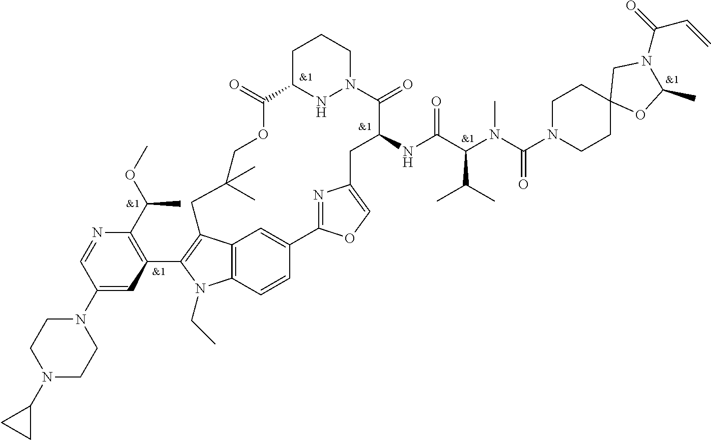 |
| A340 | 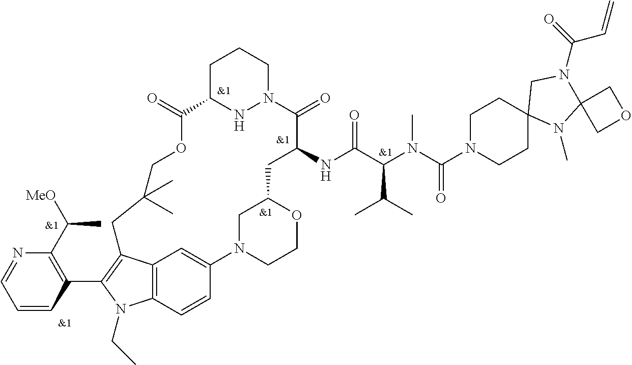 |
| A341 | 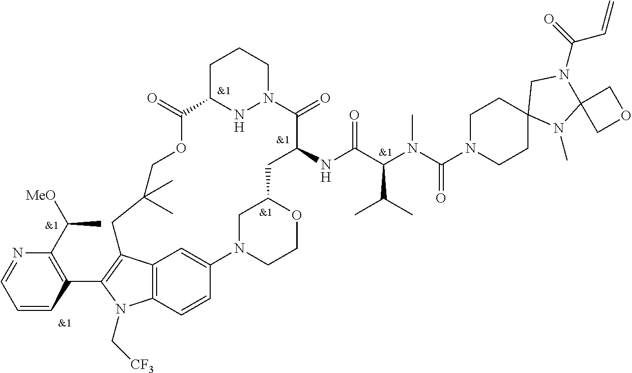 |
| A342 | 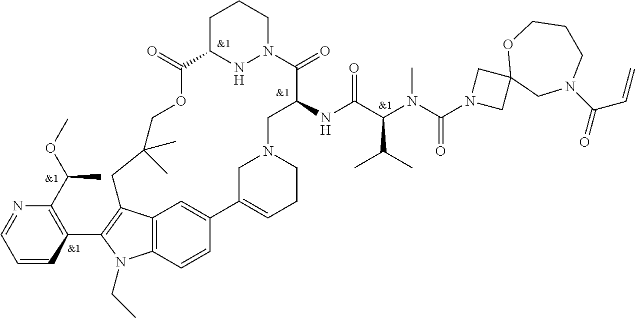 |
| A343 | 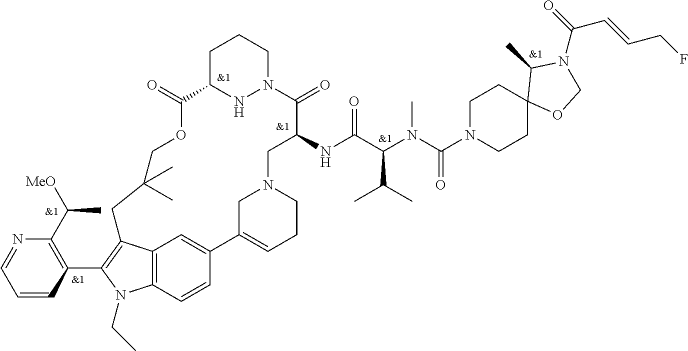 |
| A344 | 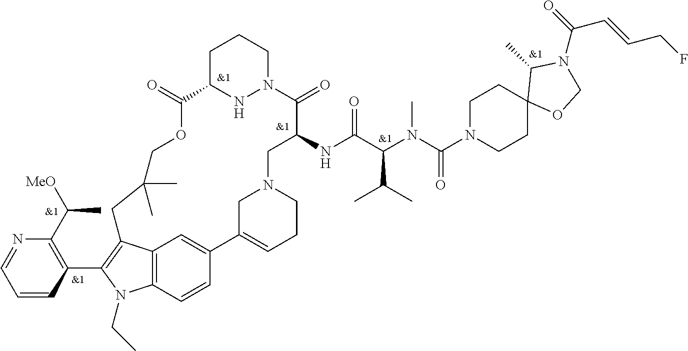 |
| A345 | 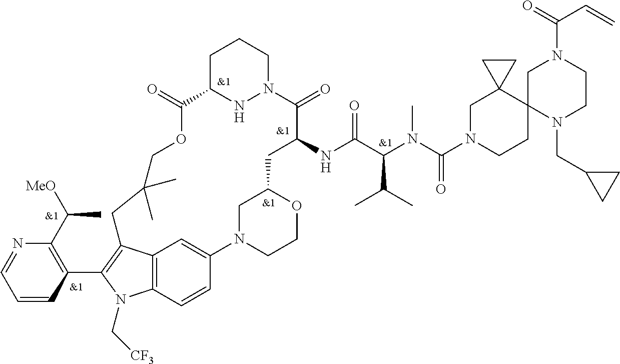 |
| A346 | 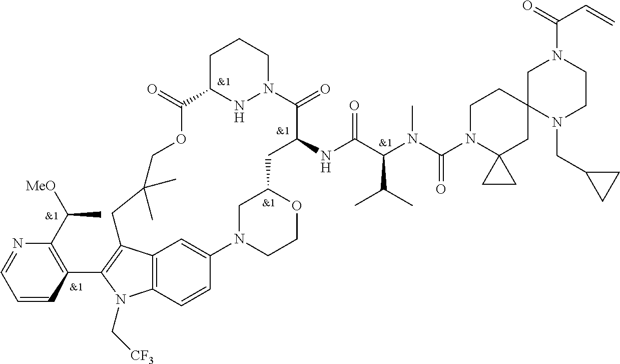 |
| A347 | 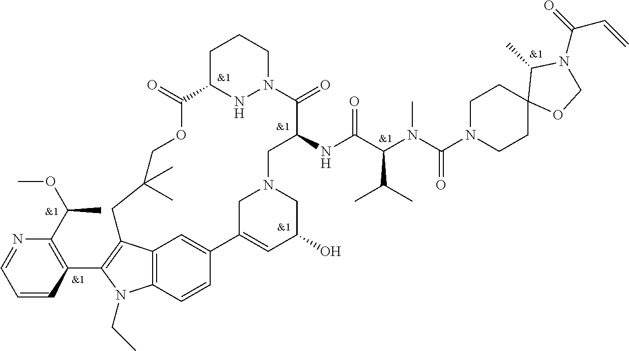 |
| A348 | 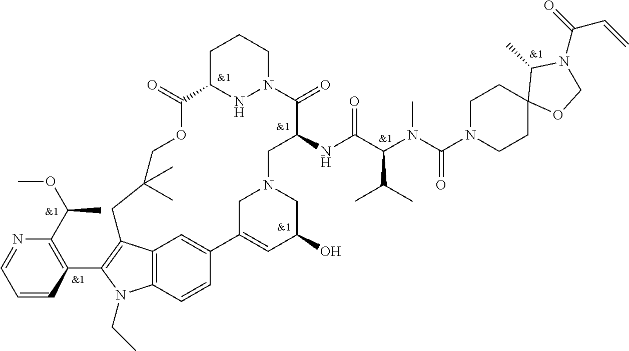 |
| A349 | 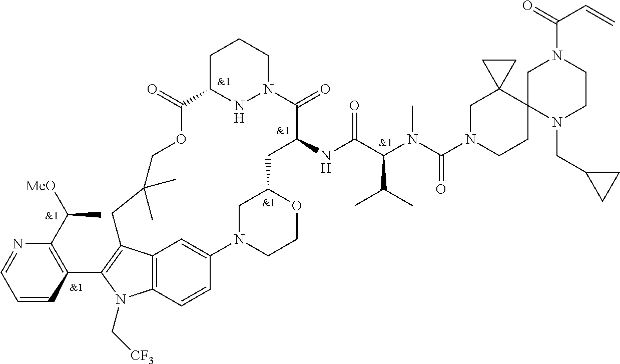 |
| A350 | 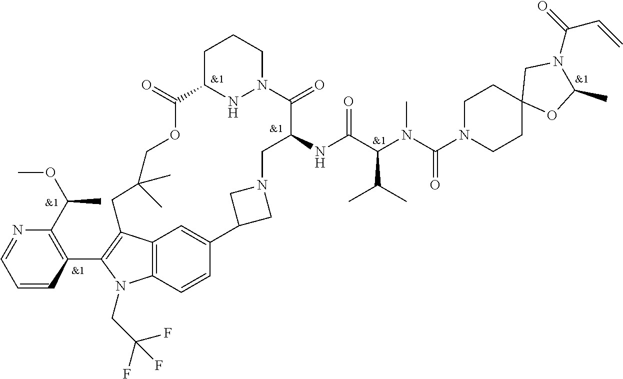 |
| A351 | 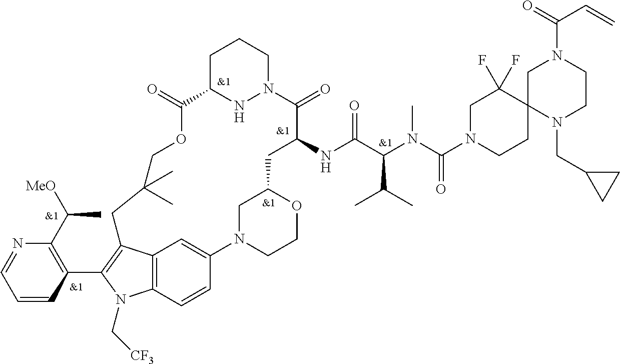 |
| A352 | 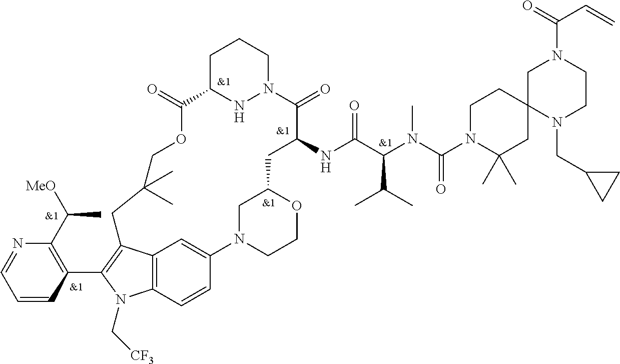 |
| A353 | 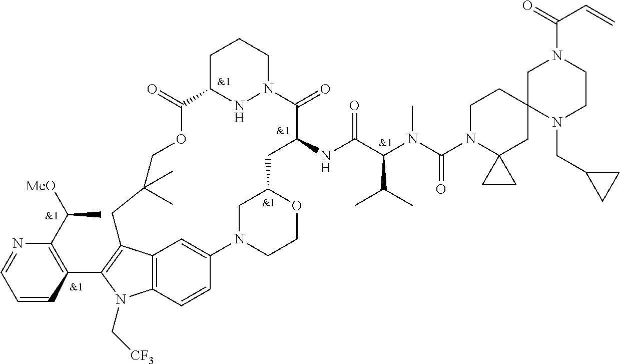 |
| A354 | 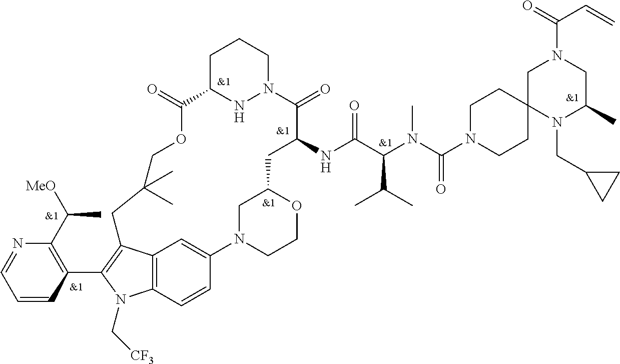 |
| A355 | 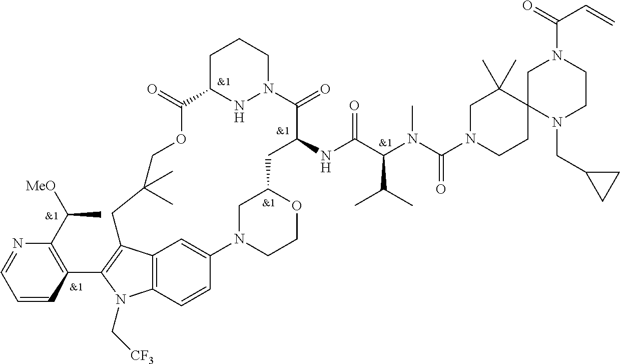 |
| A356 | 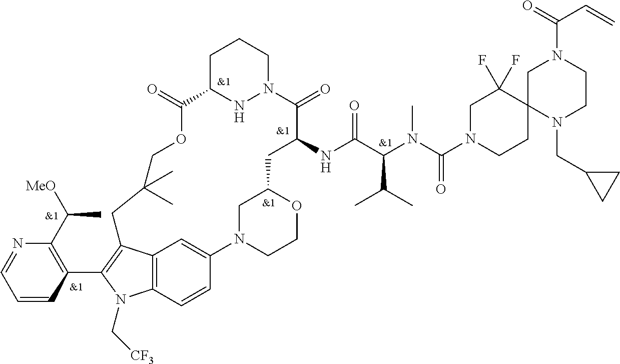 |
| A357 | 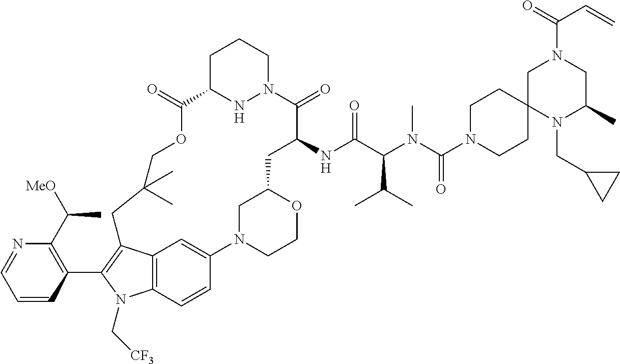 |
| A358 | 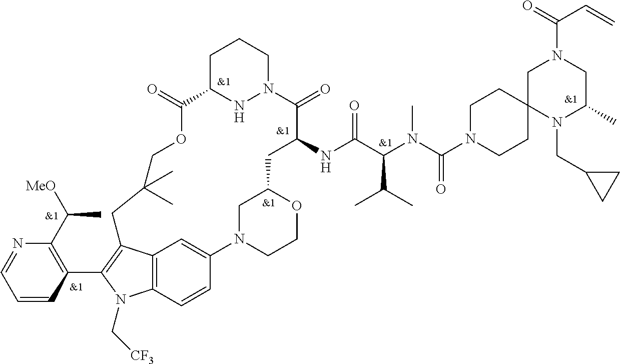 |
| A359 | 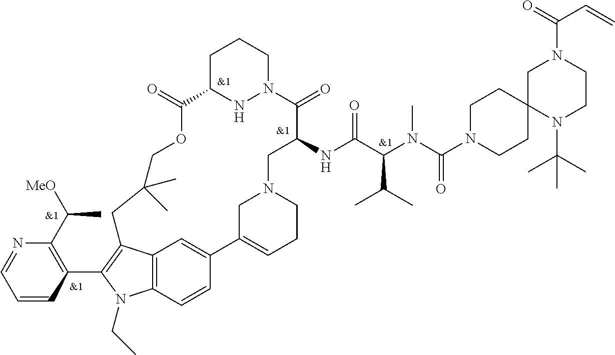 |
| A360 | 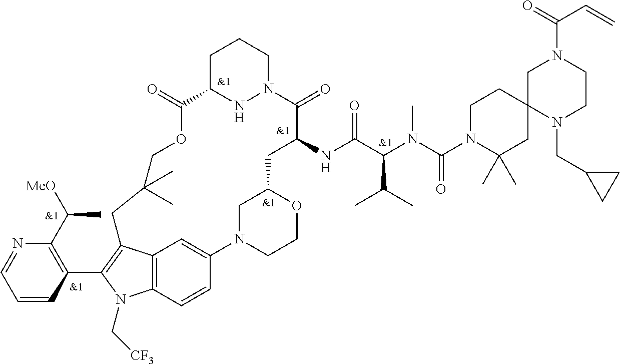 |
| A361 | 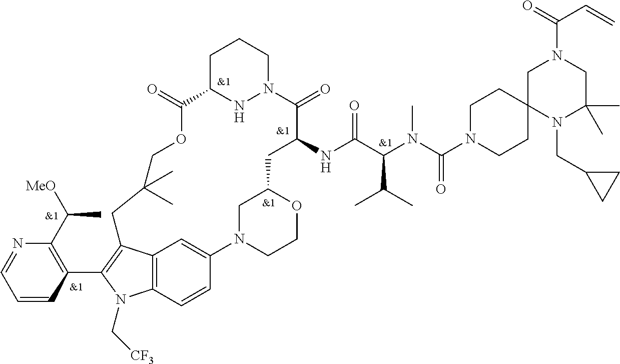 |
| A362 | 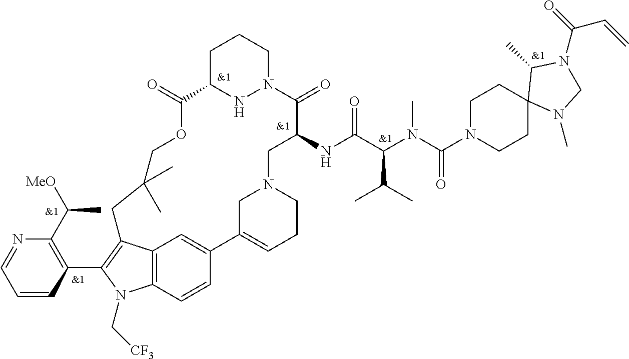 |
| A363 | 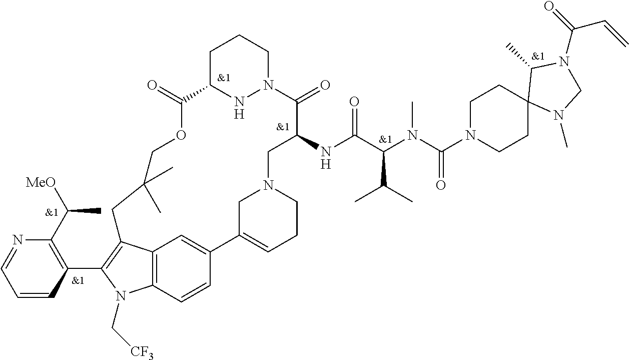 |
| A364 | 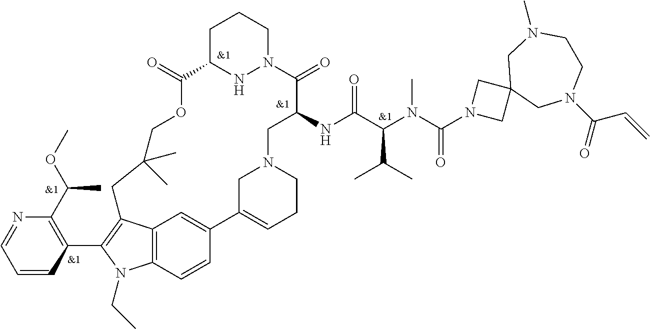 |
| A365 | 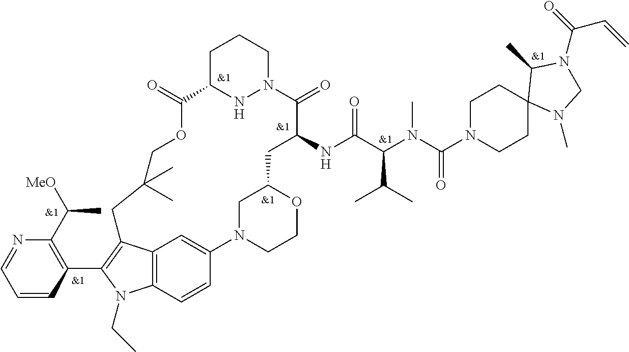 |
| A366 | 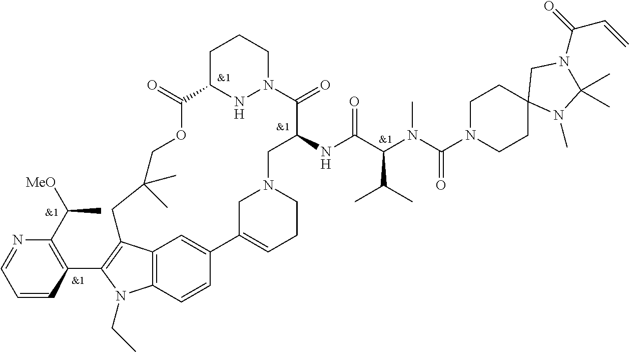 |
| A367 | 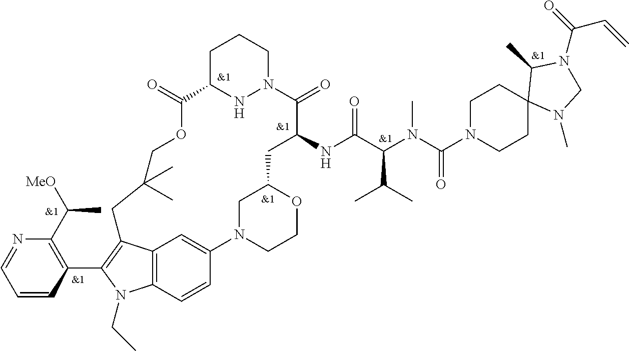 |
| A368 | 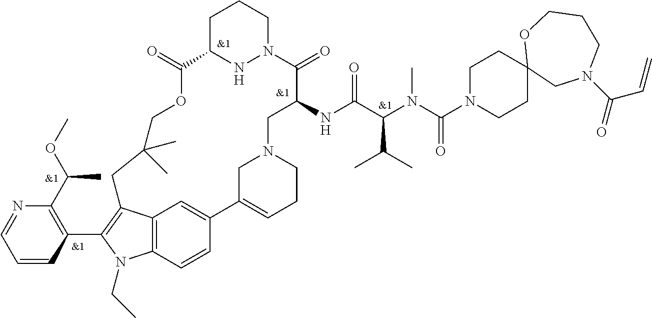 |

In some embodiments, a compound of the present invention is a compound selected from Table 2, or a pharmaceutically acceptable salt or stereoisomer thereof. In some embodiments, a compound of the present invention is a compound selected from Table 2, or a pharmaceutically acceptable salt or atropisomer thereof.

In some embodiments, a compound of the present invention is not a compound selected from Table 2. In some embodiments, a compound of the present invention is not a compound selected from Table 2, or a pharmaceutically acceptable salt or stereoisomer thereof. In some embodiments, a compound of the present invention is not a compound selected from Table 2, or a pharmaceutically acceptable salt or atropisomer thereof.

Lengthy table referenced here
US20250197423A1-20250619-T00001
Please refer to the end of the specification for access instructions.

Note that some compounds are shown with bonds as flat or wedged. In some instances, the relative stereochemistry of stereoisomers has been determined; in some instances, the absolute stereochemistry has been determined. In some instances, a single Example number corresponds to a mixture of stereoisomers. All stereoisomers of the compounds of the foregoing table are contemplated by the present invention. In particular embodiments, an atropisomer of a compound of the foregoing table is contemplated. Brackets are to be ignored.

Also provided is a pharmaceutical composition comprising a compound of the present invention, or a pharmaceutically acceptable salt thereof, and a pharmaceutically acceptable excipient.

Further provided is a conjugate, or salt thereof, comprising the structure of Formula V:

M-L-P Formula V,
wherein L is a linker;
P is a monovalent organic moiety; and
M has the structure of Formula VIa:

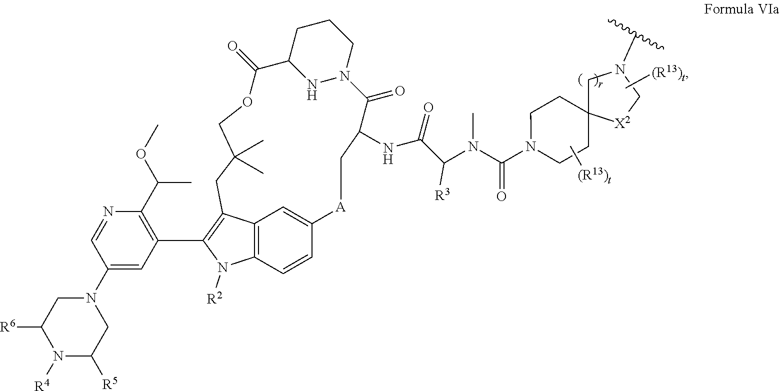

wherein A is optionally substituted 3 to 6-membered heterocycloalkylene, optionally substituted 3 to 6-membered cycloalkylene, optionally substituted 6-membered arylene, or optionally substituted 5 to 10-membered heteroarylene;
R² is optionally substituted C₁-C₆ alkyl;
R³ is optionally substituted C₁-C₆ alkyl or optionally substituted C₁-C₃ heteroalkyl;
X² is O, C(R¹¹)₂, NR¹², S, or SO₂;
r is 1 or 2;
each t is, independently, 0, 1, or 2;
R¹¹ and R¹² are each, independently, hydrogen, optionally substituted C₁-C₄ alkyl, optionally substituted C₂-C₄ heteroalkyl, or optionally substituted 3 to 5-membered cycloalkyl;
each R¹³ is, independently, —CH₃; and
R⁴, R⁵, and R⁶ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered heterocycloalkyl; or
R⁴ and R⁵ combine with the atoms to which they are attached to form an optionally substituted 3 to 8-membered cycloalkyl or an optionally substituted 3 to 8-membered heterocycloalkyl; or
R⁴ and R⁶ combine with the atoms to which they are attached to form an optionally substituted 3 to 8-membered cycloalkyl or an optionally substituted 3 to 8-membered heterocycloalkyl.

Further provided is a conjugate, or salt thereof, comprising the structure of Formula V:

M-L-P Formula V,
wherein L is a linker;
P is a monovalent organic moiety; and
M has the structure of Formula VIb:

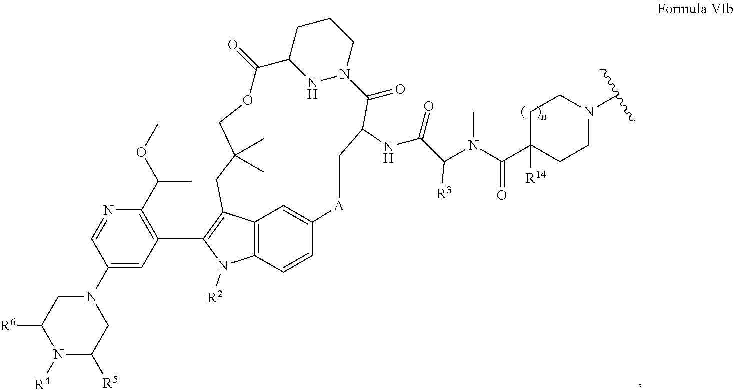

wherein A is optionally substituted 3 to 6-membered heterocycloalkylene, optionally substituted 3 to 6-membered cycloalkylene, optionally substituted 6-membered arylene, or optionally substituted 5 to 10-membered heteroarylene;
R² is optionally substituted C₁-C₆ alkyl;
R³ is optionally substituted C₁-C₆ alkyl or optionally substituted C₁-C₃ heteroalkyl;
R¹⁴ is fluoro, hydrogen, or C₁-C₃ alkyl;
u is 0 or 1; and
R⁴, R⁵, and R⁶ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered heterocycloalkyl; or
R⁴ and R⁵ combine with the atoms to which they are attached to form an optionally substituted 3 to 8-membered cycloalkyl or an optionally substituted 3 to 8-membered heterocycloalkyl; or
R⁴ and R⁶ combine with the atoms to which they are attached to form an optionally substituted 3 to 8-membered cycloalkyl or an optionally substituted 3 to 8-membered heterocycloalkyl.

Further provided is a conjugate, or salt thereof, comprising the structure of Formula V:

M-L-P Formula V,
wherein L is a linker;
P is a monovalent organic moiety; and
M has the structure of Formula VIc:

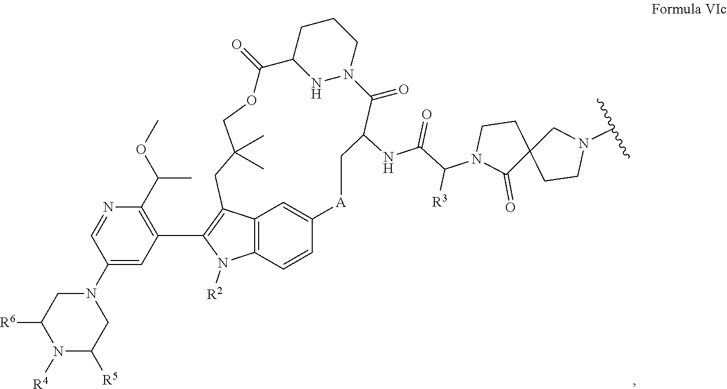

wherein A is optionally substituted 3 to 6-membered heterocycloalkylene, optionally substituted 3 to 6-membered cycloalkylene, optionally substituted 6-membered arylene, or optionally substituted 5 to 10-membered heteroarylene;
R² is optionally substituted C₁-C₆ alkyl;
R³ is optionally substituted C₁-C₆ alkyl or optionally substituted C₁-C₃ heteroalkyl; and
R⁴, R⁵, and R⁶ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered heterocycloalkyl; or
R⁴ and R⁵ combine with the atoms to which they are attached to form an optionally substituted 3 to 8-membered cycloalkyl or an optionally substituted 3 to 8-membered heterocycloalkyl; or
R⁴ and R⁶ combine with the atoms to which they are attached to form an optionally substituted 3 to 8-membered cycloalkyl or an optionally substituted 3 to 8-membered heterocycloalkyl.

Further provided is a conjugate, or salt thereof, comprising the structure of Formula V:

M-L-P Formula V,
wherein L is a linker;
P is a monovalent organic moiety; and
M has the structure of Formula VId:

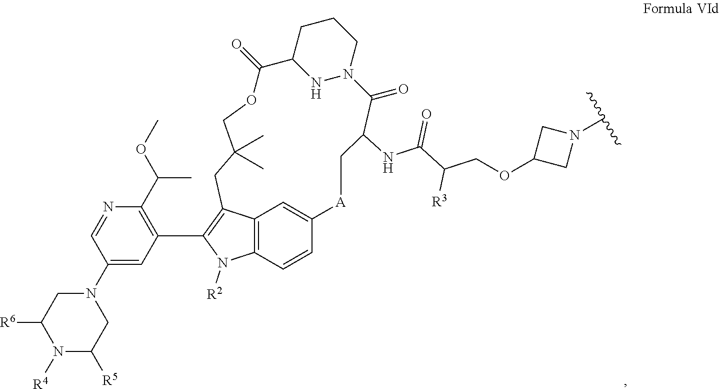

wherein A is optionally substituted 3 to 6-membered heterocycloalkylene, optionally substituted 3 to 6-membered cycloalkylene, optionally substituted 6-membered arylene, or optionally substituted 5 to 10-membered heteroarylene;
R² is optionally substituted C₁-C₆ alkyl;
R³ is optionally substituted C₁-C₆ alkyl or optionally substituted C₁-C₃ heteroalkyl; and
R⁴, R⁵, and R⁶ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered heterocycloalkyl; or
R⁴ and R⁵ combine with the atoms to which they are attached to form an optionally substituted 3 to 8-membered cycloalkyl or an optionally substituted 3 to 8-membered heterocycloalkyl; or
R⁴ and R⁶ combine with the atoms to which they are attached to form an optionally substituted 3 to 8-membered cycloalkyl or an optionally substituted 3 to 8-membered heterocycloalkyl.

In some embodiments of conjugates of the present invention, the monovalent organic moiety is a protein. In some embodiments, the protein is a Ras protein. In some embodiments, the Ras protein is K-Ras G12C, K-Ras G13C, H-Ras G12C, H-Ras G13C, N-Ras G12C, or N-Ras G13C. In some embodiments of conjugates of the present invention, the linker is bound to the monovalent organic moiety through a bond to a sulfhydryl group of an amino acid residue of the monovalent organic moiety.

Compounds of the present invention are also adaptable for uses in antibody-drug conjugates as well as degrader applications.

Further provided is a method of treating cancer in a subject in need thereof, the method comprising administering to the subject a therapeutically effective amount of a compound of the present invention, or a pharmaceutically acceptable salt thereof. The cancer may, for example, be pancreatic cancer, colorectal cancer, non-small cell lung cancer, acute myeloid leukemia, multiple myeloma, thyroid gland adenocarcinoma, a myelodysplastic syndrome, or squamous cell lung carcinoma. In some embodiments, the cancer comprises a Ras mutation, such as K-Ras G12C, K-Ras G13C, H-Ras G12C, H-Ras G13C, N-Ras G12C, or N-Ras G13C. Other Ras mutations are described herein.

Further provided is a method of treating a Ras protein-related disorder in a subject in need thereof, the method comprising administering to the subject a therapeutically effective amount of a compound of the present invention, or a pharmaceutically acceptable salt thereof.

Further provided is a method of inhibiting a Ras protein in a cell, the method comprising contacting the cell with an effective amount of a compound of the present invention, or a pharmaceutically acceptable salt thereof. For example, the Ras protein is K-Ras G12C, K-Ras G13C, H-Ras G12C, H-Ras G13C, N-Ras G12C, or N-Ras G13C. Other Ras proteins are described herein. The cell may be a cancer cell, such as a pancreatic cancer cell, a colorectal cancer cell, a non-small cell lung cancer cell, an acute myeloid leukemia cell, a multiple myeloma cell, a thyroid gland adenocarcinoma cell, a myelodysplastic syndrome cell, or a squamous cell lung carcinoma cell. Other cancer types are described herein. The cell may be in vivo or in vitro.

Further provided is a method of treating a K-Ras G13C mutant cancer with a compound of Formula I. Further provided is a method of treating a K-Ras G13C mutant cancer with a compound of Formula Ia. Further provided is a method of treating a K-Ras G13C mutant cancer with a compound of Formula II-1. Further provided is a method of treating a K-Ras G13C mutant cancer with a compound of Formula II-2. Further provided is a method of treating a K-Ras G13C mutant cancer with a compound of Formula II-3. Further provided is a method of treating a K-Ras G13C mutant cancer with a compound of Formula II-4. Further provided is a method of treating a K-Ras G13C mutant cancer with a compound of Formula II-5. Further provided is a method of treating a K-Ras G13C mutant cancer with a compound of Formula II-5a. Further provided is a method of treating a K-Ras G13C mutant cancer with a compound of Formula II-6. Further provided is a method of treating a K-Ras G13C mutant cancer with a compound of Formula II-6a. Further provided is a method of treating a K-Ras G13C mutant cancer with a compound of Formula II-6b. Further provided is a method of treating a K-Ras G13C mutant cancer with a compound of Formula II-6c.

Further provided is a method of treating a K-Ras G12C mutant cancer with a compound of Formula I. Further provided is a method of treating a K-Ras G12C mutant cancer with a compound of Formula Ia. Further provided is a method of treating a K-Ras G12C mutant cancer with a compound of Formula II-1. Further provided is a method of treating a K-Ras G12C mutant cancer with a compound of Formula II-2. Further provided is a method of treating a K-Ras G12C mutant cancer with a compound of Formula II-3. Further provided is a method of treating a K-Ras G12C mutant cancer with a compound of Formula II-4. Further provided is a method of treating a K-Ras G12C mutant cancer with a compound of Formula II-5. Further provided is a method of treating a K-Ras G12C mutant cancer with a compound of Formula II-5a. Further provided is a method of treating a K-Ras G12C mutant cancer with a compound of Formula II-6. Further provided is a method of treating a K-Ras G12C mutant cancer with a compound of Formula II-6a. Further provided is a method of treating a K-Ras G12C mutant cancer with a compound of Formula II-6b. Further provided is a method of treating a K-Ras G12C mutant cancer with a compound of Formula II-6c.

With respect to compounds of the present invention, one stereoisomer may exhibit better inhibition than another stereoisomer. For example, one atropisomer may exhibit inhibition, whereas the other atropisomer may exhibit little or no inhibition.

In some embodiments, a method or use described herein further comprises administering an additional anti-cancer therapy. In some embodiments, the additional anti-cancer therapy is a HER2 inhibitor, an EGFR inhibitor, a second Ras inhibitor, a SHP2 inhibitor, an SOS1 inhibitor, a Raf inhibitor, a MEK inhibitor, an ERK inhibitor, a PI3K inhibitor, a PTEN inhibitor, an AKT inhibitor, an mTORC1 inhibitor, a BRAF inhibitor, a PD-L1 inhibitor, a PD-1 inhibitor, a CDK4/6 inhibitor, or a combination thereof. In some embodiments, the additional anticancer therapy is a SHP2 inhibitor. Other additional anti-cancer therapies are described herein.

### Methods of Synthesis

The compounds described herein may be made from commercially available starting materials or synthesized using known organic, inorganic, or enzymatic processes.

The compounds of the present invention can be prepared in a number of ways well known to those skilled in the art of organic synthesis. By way of example, compounds of the present invention can be synthesized using the methods described in the Schemes below, together with synthetic methods known in the art of synthetic organic chemistry, or variations thereon as appreciated by those skilled in the art. These methods include but are not limited to those methods described in the Schemes below.

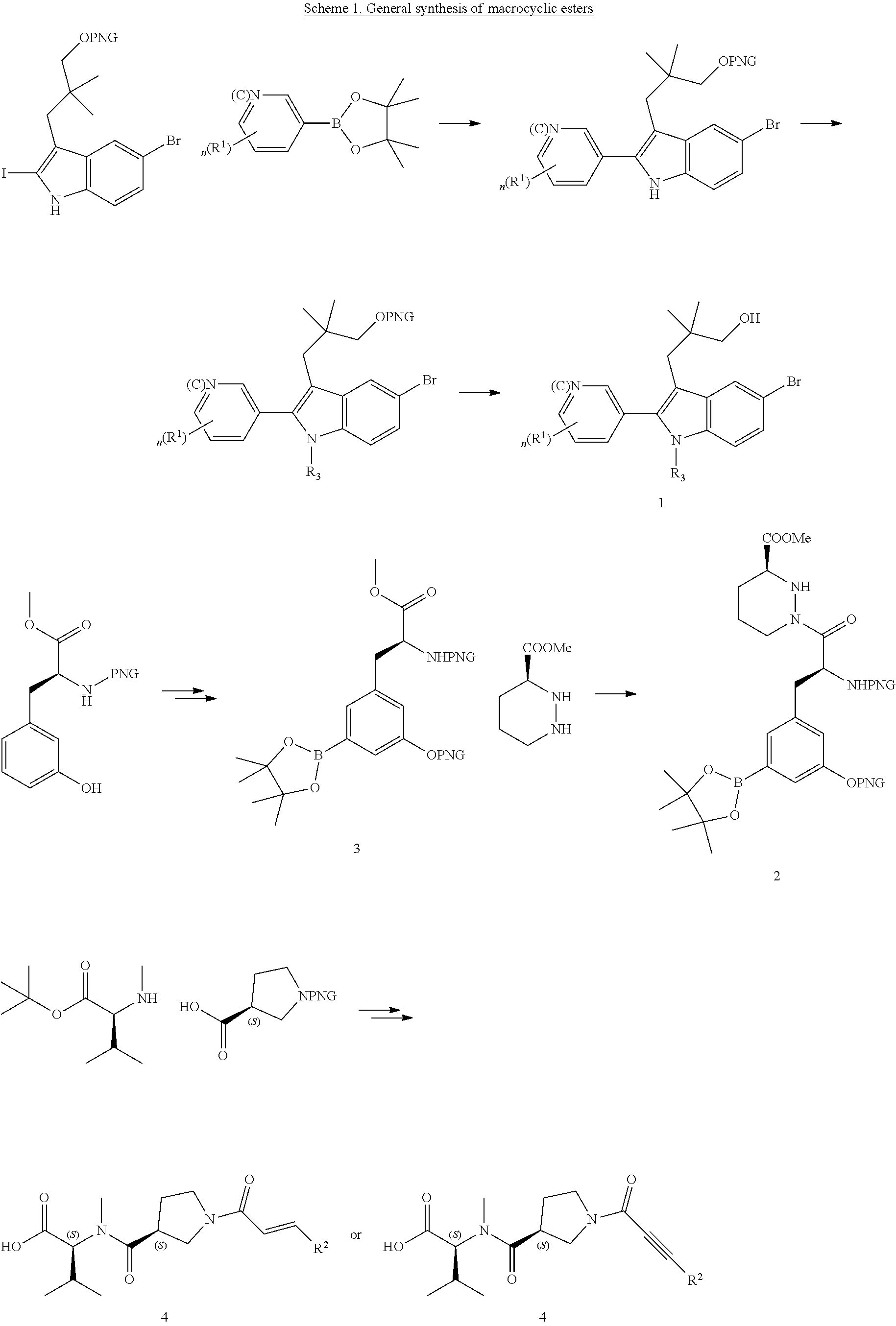

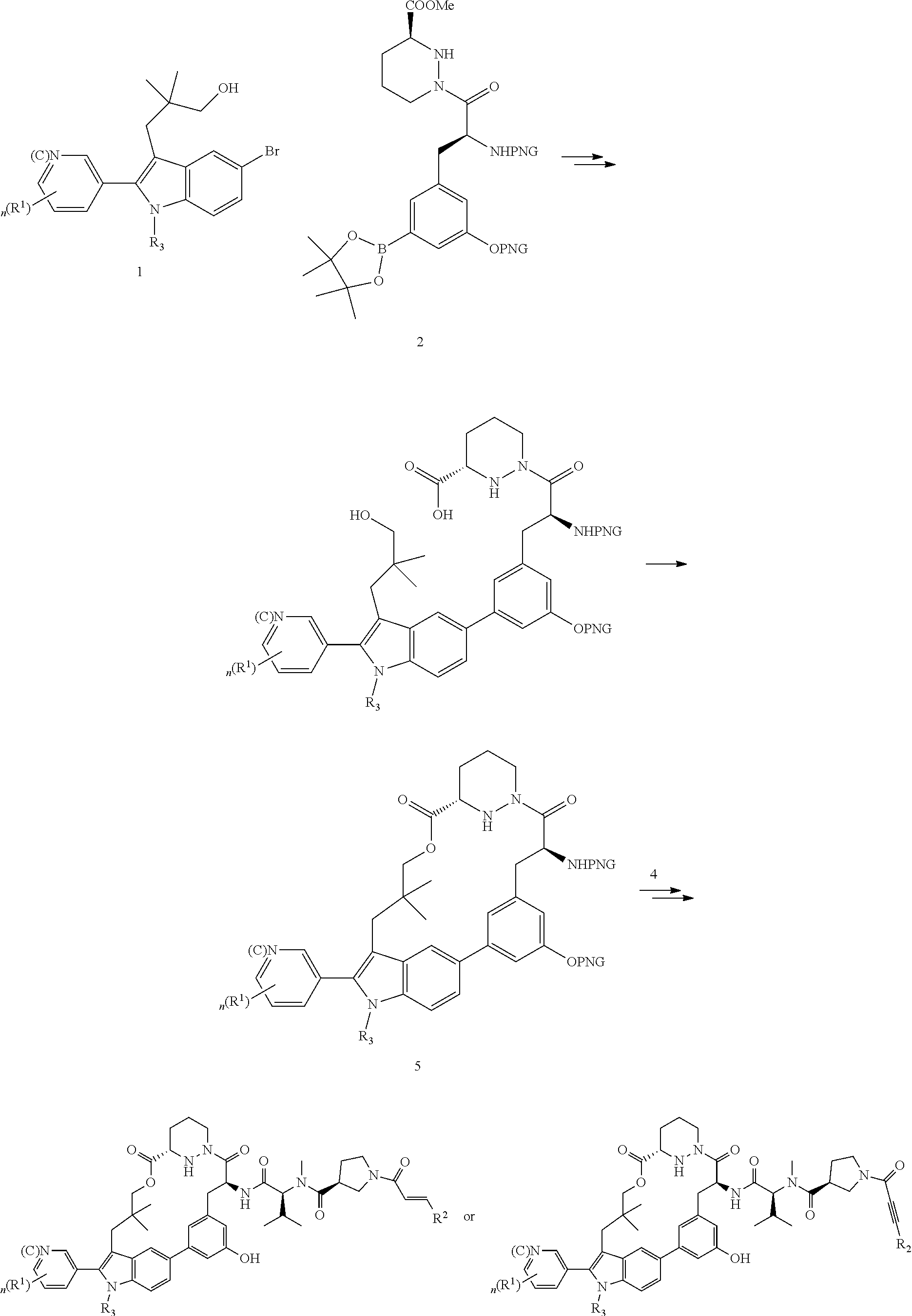

A general synthesis of macrocyclic esters is outlined in Scheme 1. An appropriately substituted aryl-3-(5-bromo-1-ethyl-1H-indol-3-yl)-2,2-dimethylpropan-1-ol (1) can be prepared in three steps starting from protected 3-(5-bromo-2-iodo-1H-indol-3-yl)-2,2-dimethylpropan-1-ol and appropriately substituted boronic acid, including palladium mediated coupling, alkylation, and de-protection reactions. Methyl-amino-hexahydropyridazine-3-carboxylate-boronic ester (2) can be prepared in three steps, including protection, iridium catalyst mediated borylation, and coupling with methyl methyl (S)-hexahydropyridazine-3-carboxylate.

An appropriately substituted acetylpyrrolidine-3-carbonyl-N-methyl-L-valine (or an alternative amino acid derivative (4) can be made by coupling of methyl-L-valinate and protected(S)-pyrrolidine-3-carboxylic acid, followed by deprotection, coupling with a carboxylic acid containing an appropriately substituted Michael acceptor, and a hydrolysis step.

The final macrocyclic esters can be made by coupling of methyl-amino-hexahydropyridazine-3-carboxylate-boronic ester (2) and aryl-3-(5-bromo-1-ethyl-1H-indol-3-yl)-2,2-dimethylpropan-1-ol (1) in the presence of a Pd catalyst followed by hydrolysis and macrolactonization steps to result in an appropriately protected macrocyclic intermediate (5). Deprotection and coupling with an appropriately substituted intermediate 4 results in a macrocyclic product. Additional deprotection and/or functionalization steps can be required to produce the final compound.

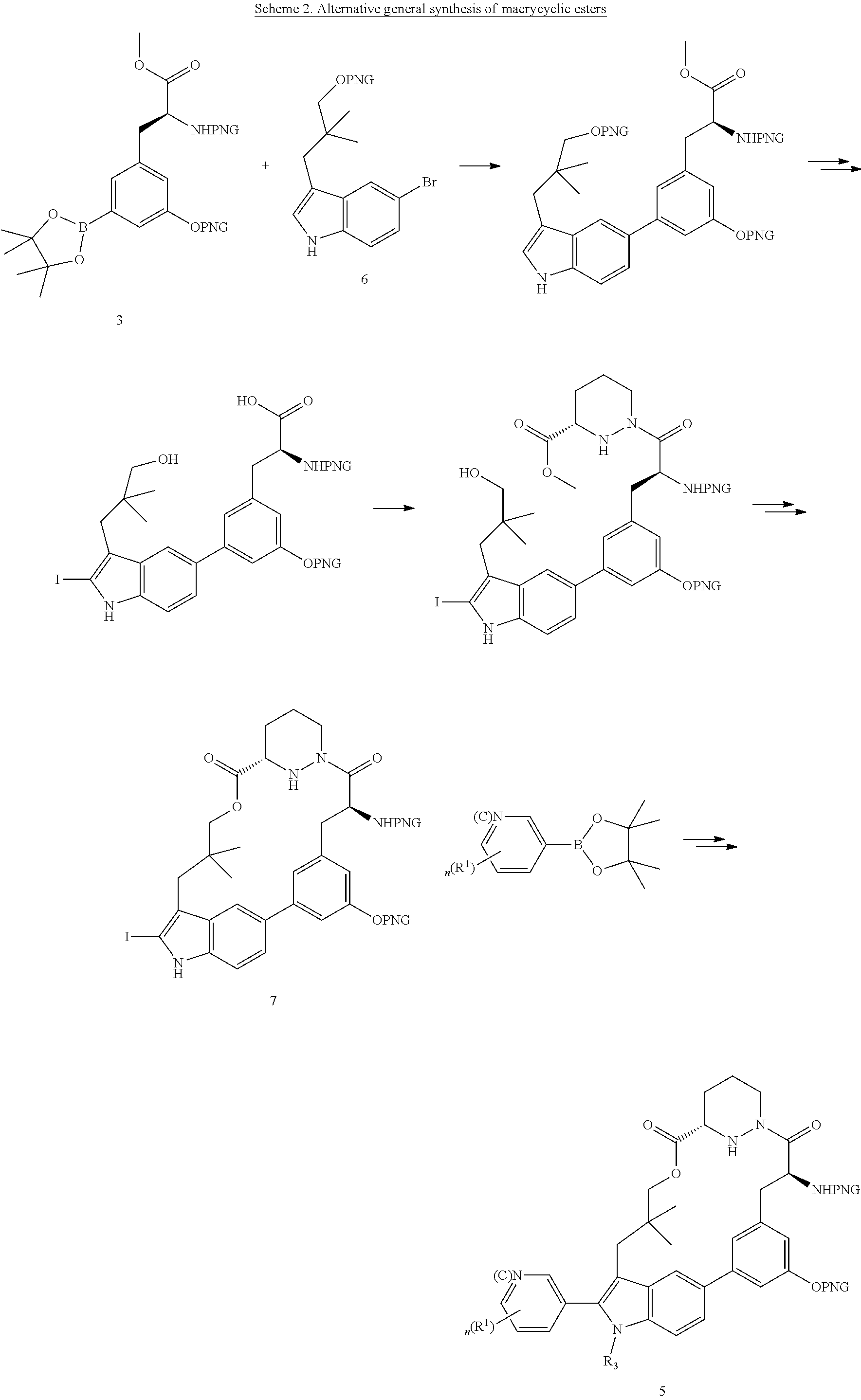

Alternatively, macrocyclic ester can be prepared as described in Scheme 2. An appropriately protected bromo-indolyl (6) coupled in the presence of a Pd catalyst with boronic ester (3), followed by iodination, deprotection, and ester hydrolysis. Subsequent coupling with methyl (S)-hexahydropyridazine-3-carboxylate, followed by hydrolysis and macrolactonization can result in iodo intermediate (7). Coupling in the presence of a Pd catalyst with an appropriately substituted boronic ester and alkylation can yield fully protected macrocycle (5). Additional deprotection or functionalization steps are required to produce the final compound.

In addition, compounds of the disclosure can be synthesized using the methods described in the Examples below, together with synthetic methods known in the art of synthetic organic chemistry, or variations thereon as appreciated by those skilled in the art. These methods include but are not limited to those methods described in the Examples below. For example, a person of skill in the art would be able to install into a macrocyclic ester a desired-B-L-W group of a compound of Formula (I), where B, L and W are defined herein, including by using methods exemplified in the Example section herein.

Compounds of Table 1 herein were prepared using methods disclosed herein or were prepared using methods disclosed herein combined with the knowledge of one of skill in the art. Compounds of Table 2 may be prepared using methods disclosed herein or may be prepared using methods disclosed herein combined with the knowledge of one of skill in the art.

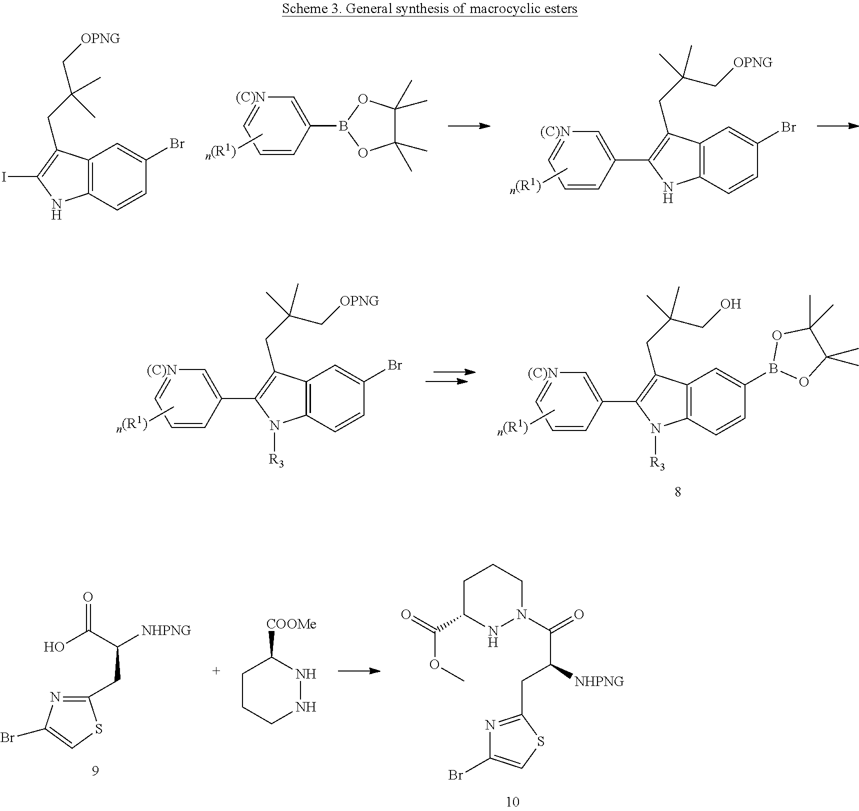

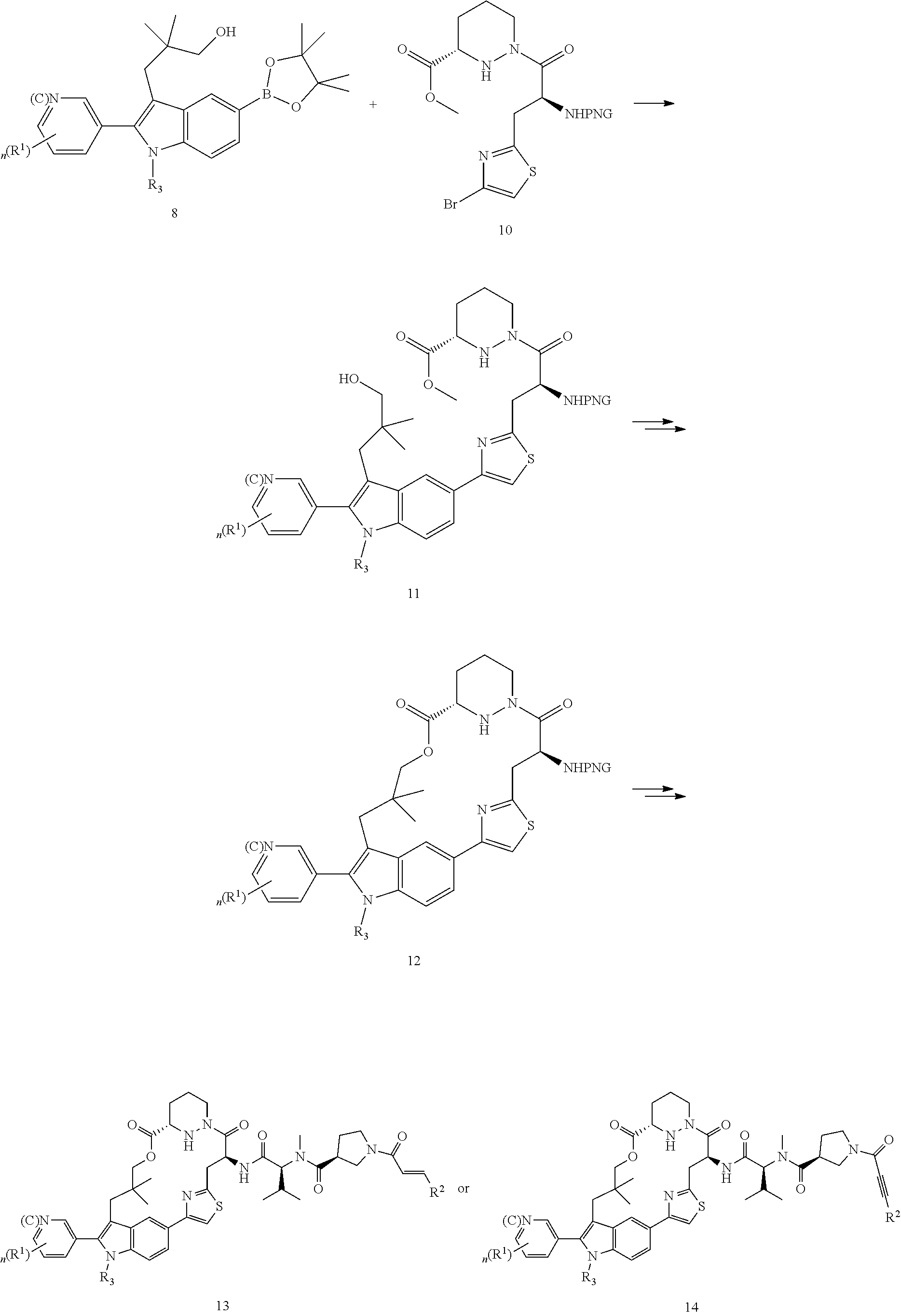

An alternative general synthesis of macrocyclic esters is outlined in Scheme 3. An appropriately substituted indolyl boronic ester (8) can be prepared in four steps starting from protected 3-(5-bromo-2-iodo-1H-indol-3-yl)-2,2-dimethylpropan-1-ol and appropriately substituted boronic acid, including Palladium mediated coupling, alkylation, de-protection, and Palladium mediated borylation reactions.

Methyl-amino-3-(4-bromothiazol-2-yl)propanoyl)hexahydropyridazine-3-carboxylate (10) can be prepared via coupling of (S)-2-amino-3-(4-bromothiazol-2-yl)propanoic acid (9) with methyl (S)-hexahydropyridazine-3-carboxylate.

The final macrocyclic esters can be made by coupling of Methyl-amino-3-(4-bromothiazol-2-yl)propanoyl)hexahydropyridazine-3-carboxylate (10) and an appropriately substituted indolyl boronic ester (8) in the presence of Pd catalyst followed by hydrolysis and macrolactonization steps to result in an appropriately protected macrocyclic intermediate (11). Deprotection and coupling with an appropriately substituted intermediate 4 can result in a macrocyclic product. Additional deprotection or functionalization steps could be required to produce a final compound 13 or 14.

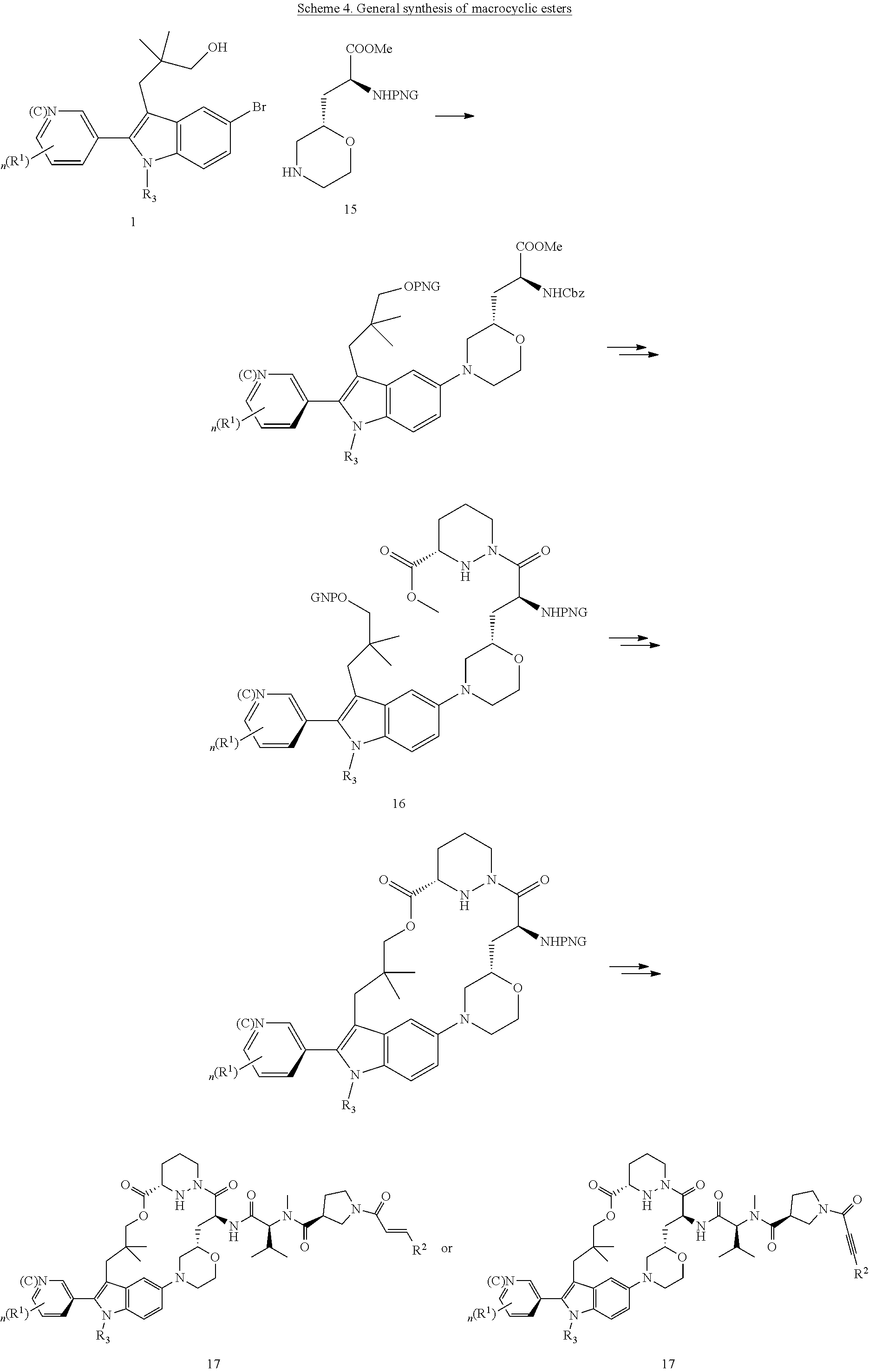

An alternative general synthesis of macrocyclic esters is outlined in Scheme 4. An appropriately substituted morpholine or an alternative herecyclic intermediate (15) can be coupled with appropriately protected Intermediate 1 via Palladium mediated coupling. Subsequent ester hydrolysis and coupling with piperazoic ester results in intermediate 16.

The macrocyclic esters can be made by hydrolysis, deprotection and macrocyclization sequence. Subsequent deprotection and coupling with Intermediate 4 (or analogs) result in an appropriately substituted final macrocyclic products. Additional deprotection or functionalization steps could be required to produce a final compound 17.

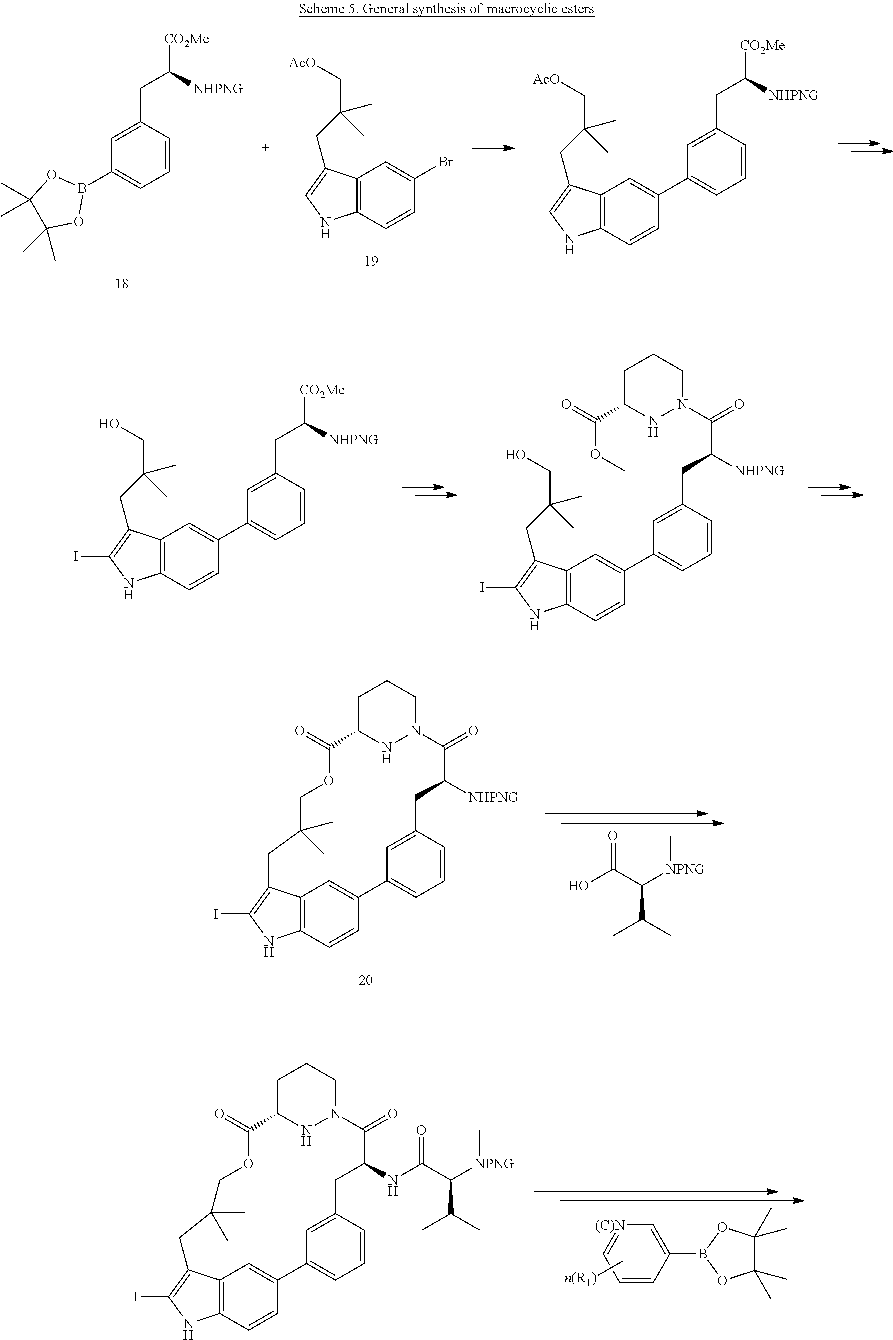

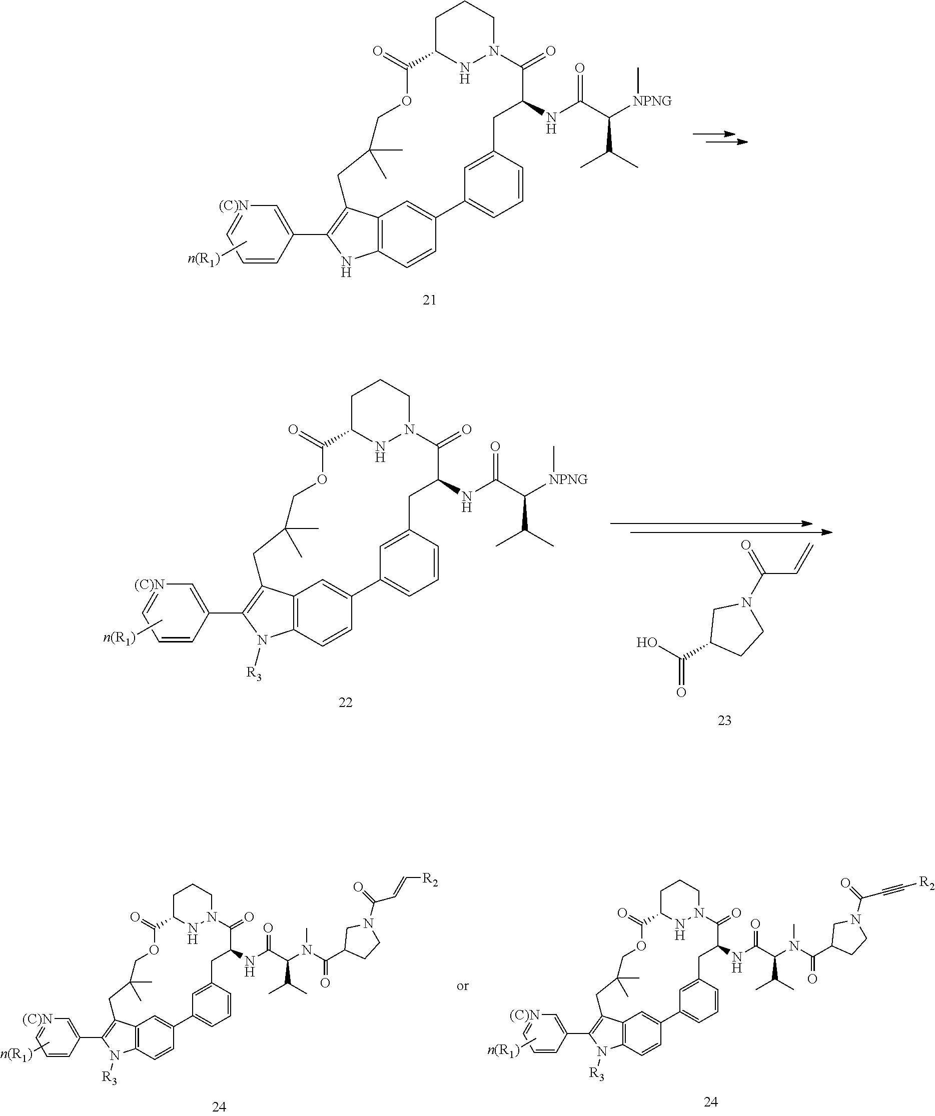

An alternative general synthesis of macrocyclic esters is outlined in Scheme 5. An appropriately substituted macrocycle (20) can be prepared starting from an appropriately protected boronic ester 18 and bromo indolyl intermediate (19), including Palladium mediated coupling, hydrolysis, coupling with piperazoic ester, hydrolysis, de-protection, and macrocyclizarion steps. Subsequent coupling with an appropriately substituted protected amino acid followed by palladium mediated coupling yiels intermediate 21. Additional deprotection and derivatization steps, including alkylation may be required at this point.

The final macrocyclic esters can be made by coupling of intermediate (22) and an appropriately substituted carboxylic acid intermediate (23). Additional deprotection or functionalization steps could be required to produce a final compound (24).

In addition, compounds of the disclosure can be synthesized using the methods described in the Examples below, together with synthetic methods known in the art of synthetic organic chemistry, or variations thereon as appreciated by those skilled in the art. These methods include but are not limited to those methods described in the Examples below. For example, a person of skill in the art would be able to install into a macrocyclic ester a desired-B-L-W group of a compound of Formula (I), where B, L and W are defined herein, including by using methods exemplified in the Example section herein.

### Pharmaceutical Compositions and Methods of Use

### Pharmaceutical Compositions and Methods of Administration

The compounds with which the invention is concerned are Ras inhibitors and are useful in the treatment of cancer. Accordingly, one embodiment of the present invention provides pharmaceutical compositions containing a compound of the invention or a pharmaceutically acceptable salt thereof, and a pharmaceutically acceptable excipient, as well as methods of using the compounds of the invention to prepare such compositions.

As used herein, the term “pharmaceutical composition” refers to a compound, such as a compound of the present invention, or a pharmaceutically acceptable salt thereof, formulated together with a pharmaceutically acceptable excipient.

In some embodiments, a compound is present in a pharmaceutical composition in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

A “pharmaceutically acceptable excipient,” as used herein, refers to any inactive ingredient (for example, a vehicle capable of suspending or dissolving the active compound) having the properties of being nontoxic and non-inflammatory in a subject. Typical excipients include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Excipients include, but are not limited to: butylated optionally substituted hydroxyltoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, optionally substituted hydroxylpropyl cellulose, optionally substituted hydroxylpropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol. Those of ordinary skill in the art are familiar with a variety of agents and materials useful as excipients. See, e.g., e.g., Ansel, et al., Ansel's Pharmaceutical Dosage Forms and Drug Delivery Systems. Philadelphia: Lippincott, Williams & Wilkins, 2004; Gennaro, et al., Remington: The Science and Practice of Pharmacy. Philadelphia: Lippincott, Williams & Wilkins, 2000; and Rowe, Handbook of Pharmaceutical Excipients. Chicago, Pharmaceutical Press, 2005. In some embodiments, a composition includes at least two different pharmaceutically acceptable excipients.

Compounds described herein, whether expressly stated or not, may be provided or utilized in salt form, e.g., a pharmaceutically acceptable salt form, unless expressly stated to the contrary. The term “pharmaceutically acceptable salt,” as use herein, refers to those salts of the compounds described herein that are, within the scope of sound medical judgment, suitable for use in contact with the tissues of humans and other animals without undue toxicity, irritation, allergic response and the like, and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting the free base group with a suitable organic acid.

The compounds of the invention may have ionizable groups so as to be capable of preparation as pharmaceutically acceptable salts. These salts may be acid addition salts involving inorganic or organic acids or the salts may, in the case of acidic forms of the compounds of the invention, be prepared from inorganic or organic bases. In some embodiments, the compounds are prepared or used as pharmaceutically acceptable salts prepared as addition products of pharmaceutically acceptable acids or bases. Suitable pharmaceutically acceptable acids and bases are well-known in the art, such as hydrochloric, sulfuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well-established in the art.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-optionally substituted hydroxyl-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts and the like. Representative alkali or alkaline earth metal salts include sodium, lithium, potassium, calcium, magnesium and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

As used herein, the term “subject” refers to any member of the animal kingdom. In some embodiments, “subject” refers to humans, at any stage of development. In some embodiments, “subject” refers to a human patient. In some embodiments, “subject” refers to non-human animals. In some embodiments, the non-human animal is a mammal (e.g., a rodent, a mouse, a rat, a rabbit, a monkey, a dog, a cat, a sheep, cattle, a primate, or a pig). In some embodiments, subjects include, but are not limited to, mammals, birds, reptiles, amphibians, fish, or worms. In some embodiments, a subject may be a transgenic animal, genetically-engineered animal, or a clone.

As used herein, the term “dosage form” refers to a physically discrete unit of a compound (e.g., a compound of the present invention) for administration to a subject. Each unit contains a predetermined quantity of compound. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or compound administered to a particular subject is determined by one or more attending physicians and may involve administration of multiple dosage forms.

As used herein, the term “dosing regimen” refers to a set of unit doses (typically more than one) that are administered individually to a subject, typically separated by periods of time. In some embodiments, a given therapeutic compound (e.g., a compound of the present invention) has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

A “therapeutic regimen” refers to a dosing regimen whose administration across a relevant population is correlated with a desired or beneficial therapeutic outcome.

The term “treatment” (also “treat” or “treating”), in its broadest sense, refers to any administration of a substance (e.g., a compound of the present invention) that partially or completely alleviates, ameliorates, relieves, inhibits, delays onset of, reduces severity of, or reduces incidence of one or more symptoms, features, or causes of a particular disease, disorder, or condition. In some embodiments, such treatment may be administered to a subject who does not exhibit signs of the relevant disease, disorder, or condition or of a subject who exhibits only early signs of the disease, disorder, or condition. Alternatively, or additionally, in some embodiments, treatment may be administered to a subject who exhibits one or more established signs of the relevant disease, disorder, or condition. In some embodiments, treatment may be of a subject who has been diagnosed as suffering from the relevant disease, disorder, or condition. In some embodiments, treatment may be of a subject known to have one or more susceptibility factors that are statistically correlated with increased risk of development of the relevant disease, disorder, or condition.

The term “therapeutically effective amount” means an amount that is sufficient, when administered to a population suffering from or susceptible to a disease, disorder, or condition in accordance with a therapeutic dosing regimen, to treat the disease, disorder, or condition. In some embodiments, a therapeutically effective amount is one that reduces the incidence or severity of, or delays onset of, one or more symptoms of the disease, disorder, or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. It is specifically understood that particular subjects may, in fact, be “refractory” to a “therapeutically effective amount.” In some embodiments, reference to a therapeutically effective amount may be a reference to an amount as measured in one or more specific tissues (e.g., a tissue affected by the disease, disorder, or condition) or fluids (e.g., blood, saliva, serum, sweat, tears, urine). Those of ordinary skill in the art will appreciate that, in some embodiments, a therapeutically effective amount may be formulated or administered in a single dose. In some embodiments, a therapeutically effective amount may be formulated or administered in a plurality of doses, for example, as part of a dosing regimen.

For use as treatment of subjects, the compounds of the invention, or a pharmaceutically acceptable salt thereof, can be formulated as pharmaceutical or veterinary compositions. Depending on the subject to be treated, the mode of administration, and the type of treatment desired, e.g., prevention, prophylaxis, or therapy, the compounds, or a pharmaceutically acceptable salt thereof, are formulated in ways consonant with these parameters. A summary of such techniques may be found in Remington: The Science and Practice of Pharmacy, 21^{st} Edition, Lippincott Williams & Wilkins, (2005); and Encyclopedia of Pharmaceutical Technology, eds. J. Swarbrick and J. C. Boylan, 1988-1999, Marcel Dekker, New York, each of which is incorporated herein by reference.

Compositions can be prepared according to conventional mixing, granulating, or coating methods, respectively, and the present pharmaceutical compositions can contain from about 0.1% to about 99%, from about 5% to about 90%, or from about 1% to about 20% of a compound of the present invention, or pharmaceutically acceptable salt thereof, by weight or volume. In some embodiments, compounds, or a pharmaceutically acceptable salt thereof, described herein may be present in amounts totaling 1-95% by weight of the total weight of a composition, such as a pharmaceutical composition.

The composition may be provided in a dosage form that is suitable for intraarticular, oral, parenteral (e.g., intravenous, intramuscular), rectal, cutaneous, subcutaneous, topical, transdermal, sublingual, nasal, vaginal, intravesicular, intraurethral, intrathecal, epidural, aural, or ocular administration, or by injection, inhalation, or direct contact with the nasal, genitourinary, reproductive, or oral mucosa. Thus, the pharmaceutical composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, preparations suitable for iontophoretic delivery, or aerosols. The compositions may be formulated according to conventional pharmaceutical practice.

As used herein, the term “administration” refers to the administration of a composition (e.g., a compound, or a preparation that includes a compound as described herein) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal, or vitreal.

Formulations may be prepared in a manner suitable for systemic administration or topical or local administration. Systemic formulations include those designed for injection (e.g., intramuscular, intravenous, or subcutaneous injection) or may be prepared for transdermal, transmucosal, or oral administration. A formulation will generally include a diluent as well as, in some cases, adjuvants, buffers, preservatives and the like. Compounds, or a pharmaceutically acceptable salt thereof, can be administered also in liposomal compositions or as microemulsions.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

Various sustained release systems for drugs have also been devised. See, for example, U.S. Pat. No. 5,624,677.

Systemic administration may also include relatively noninvasive methods such as the use of suppositories, transdermal patches, transmucosal delivery and intranasal administration. Oral administration is also suitable for compounds of the invention, or a pharmaceutically acceptable salt thereof. Suitable forms include syrups, capsules, and tablets, as is understood in the art.

Each compound, or a pharmaceutically acceptable salt thereof, as described herein, may be formulated in a variety of ways that are known in the art. For example, the first and second agents of the combination therapy may be formulated together or separately. Other modalities of combination therapy are described herein.

The individually or separately formulated agents can be packaged together as a kit. Non-limiting examples include, but are not limited to, kits that contain, e.g., two pills, a pill and a powder, a suppository and a liquid in a vial, two topical creams, etc. The kit can include optional components that aid in the administration of the unit dose to subjects, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one subject, multiple uses for a particular subject (at a constant dose or in which the individual compounds, or a pharmaceutically acceptable salt thereof, may vary in potency as therapy progresses); or the kit may contain multiple doses suitable for administration to multiple subjects (“bulk packaging”). The kit components may be assembled in cartons, blister packs, bottles, tubes, and the like.

Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, optionally substituted hydroxylpropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Two or more compounds may be mixed together in a tablet, capsule, or other vehicle, or may be partitioned. In one example, the first compound is contained on the inside of the tablet, and the second compound is on the outside, such that a substantial portion of the second compound is released prior to the release of the first compound.

Formulations for oral use may also be provided as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Dissolution or diffusion-controlled release can be achieved by appropriate coating of a tablet, capsule, pellet, or granulate formulation of compounds, or by incorporating the compound, or a pharmaceutically acceptable salt thereof, into an appropriate matrix. A controlled release coating may include one or more of the coating substances mentioned above or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-optionally substituted hydroxylmethacrylate, methacrylate hydrogels, 1,3 butylene glycol, ethylene glycol methacrylate, or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, or halogenated fluorocarbon.

The liquid forms in which the compounds, or a pharmaceutically acceptable salt thereof, and compositions of the present invention can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Generally, when administered to a human, the oral dosage of any of the compounds of the invention, or a pharmaceutically acceptable salt thereof, will depend on the nature of the compound, and can readily be determined by one skilled in the art. A dosage may be, for example, about 0.001 mg to about 2000 mg per day, about 1 mg to about 1000 mg per day, about 5 mg to about 500 mg per day, about 100 mg to about 1500 mg per day, about 500 mg to about 1500 mg per day, about 500 mg to about 2000 mg per day, or any range derivable therein.

In some embodiments, the pharmaceutical composition may further comprise an additional compound having antiproliferative activity. Depending on the mode of administration, compounds, or a pharmaceutically acceptable salt thereof, will be formulated into suitable compositions to permit facile delivery. Each compound, or a pharmaceutically acceptable salt thereof, of a combination therapy may be formulated in a variety of ways that are known in the art. For example, the first and second agents of the combination therapy may be formulated together or separately. Desirably, the first and second agents are formulated together for the simultaneous or near simultaneous administration of the agents.

It will be appreciated that the compounds and pharmaceutical compositions of the present invention can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics or procedures and the desired therapeutic effect to be achieved. It will also be appreciated that the therapies employed may achieve a desired effect for the same disorder, or they may achieve different effects (e.g., control of any adverse effects).

Administration of each drug in a combination therapy, as described herein, can, independently, be one to four times daily for one day to one year, and may even be for the life of the subject. Chronic, long-term administration may be indicated.

### Methods of Use

In some embodiments, the invention discloses a method of treating a disease or disorder that is characterized by aberrant Ras activity due to a Ras mutant. In some embodiments, the disease or disorder is a cancer.

Accordingly, also provided is a method of treating cancer in a subject in need thereof, the method comprising administering to the subject a therapeutically effective amount of a compound of the present invention, or a pharmaceutically acceptable salt thereof, or a pharmaceutical composition comprising such a compound or salt. In some embodiments, the cancer is colorectal cancer, non-small cell lung cancer, small-cell lung cancer, pancreatic cancer, appendiceal cancer, melanoma, acute myeloid leukemia, small bowel cancer, ampullary cancer, germ cell cancer, cervical cancer, cancer of unknown primary origin, endometrial cancer, esophagogastric cancer, GI neuroendocrine cancer, ovarian cancer, sex cord stromal tumor cancer, hepatobiliary cancer, or bladder cancer. In some embodiments, the cancer is appendiceal, endometrial or melanoma. Also provided is a method of treating a Ras protein-related disorder in a subject in need thereof, the method comprising administering to the subject a therapeutically effective amount of a compound of the present invention, or a pharmaceutically acceptable salt thereof, or a pharmaceutical composition comprising such a compound or salt.

In some embodiments, the compounds of the present invention or pharmaceutically acceptable salts thereof, pharmaceutical compositions comprising such compounds or salts, and methods provided herein may be used for the treatment of a wide variety of cancers including tumors such as lung, prostate, breast, brain, skin, cervical carcinomas, testicular carcinomas, etc. More particularly, cancers that may be treated by the compounds or salts thereof, pharmaceutical compositions comprising such compounds or salts, and methods of the invention include, but are not limited to tumor types such as astrocytic, breast, cervical, colorectal, endometrial, esophageal, gastric, head and neck, hepatocellular, laryngeal, lung, oral, ovarian, prostate, and thyroid carcinomas and sarcomas. Other cancers include, for example:
Cardiac, for example: sarcoma (angiosarcoma, fibrosarcoma, rhabdomyosarcoma, liposarcoma), myxoma, rhabdomyoma, fibroma, lipoma, and teratoma;
Lung, for example: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma;
Gastrointestinal, for example: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma);
Genitourinary tract, for example: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma);
Liver, for example: hepatoma (hepatocellular carcinoma), cholangiocarcinoma, hepatoblastoma, angiosarcoma, hepatocellular adenoma, hemangioma;
Biliary tract, for example: gall bladder carcinoma, ampullary carcinoma, cholangiocarcinoma;
Bone, for example: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma, and giant cell tumors;
Nervous system, for example: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, neurofibromatosis type 1, meningioma, glioma, sarcoma);
Gynecological, for example: uterus (endometrial carcinoma, uterine carcinoma, uterine corpus endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors. Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma);
Hematologic, for example: blood (myeloid leukemia (acute and chronic), acute lymphoblastic leukemia, chronic lymphocytic leukemia, myeloproliferative diseases (e.g., myelofibrosis and myeloproliferative neoplasms), multiple myeloma, myelodysplastic syndrome), Hodgkin's disease, non-Hodgkin's lymphoma (malignant lymphoma);
Skin, for example: malignant melanoma, basal cell carcinoma, squamous cell carcinoma, Kaposi's sarcoma, moles dysplastic nevi, lipoma, angioma, dermatofibroma, keloids, psoriasis; and
Adrenal glands, for example: neuroblastoma.

In some embodiments, the Ras protein is wild type (Ras^{WT}). Accordingly, in some embodiments, a compound of the present invention is employed in a method of treating a patient having a cancer comprising a Ras^{WT} (e.g., K-Ras^{WT}, H-Ras^{WT} or N-Ras^{WT}). In some embodiments, the Ras protein is Ras amplification (e.g., K-Ras^{amp}). Accordingly, in some embodiments, a compound of the present invention is employed in a method of treating a patient having a cancer comprising a Ras^{amp} (K-Ras^{amp}, H-Ras^{amp} or N-Ras^{amp}). In some embodiments, the cancer comprises a Ras mutation, such as a Ras mutation described herein. In some embodiments, a mutation is selected from:
(a) the following K-Ras mutants: G12D, G12V, G12C, G13D, G12R, G12A, Q61H, G12S, A146T, G13C, Q61L, Q61R, K117N, A146V, G12F, Q61K, L19F, Q22K, V141, A59T, A146P, G13R, G12L, or G13V, and combinations thereof;
(b) the following H-Ras mutants: Q61R, G13R, Q61K, G12S, Q61L, G12D, G13V, G13D, G12C, K117N, A59T, G12V, G13C, Q61H, G13S, A18V, D119N, G13N, A146T, A66T, G12A, A146V, G12N, or G12R, and combinations thereof; and
(c) the following N-Ras mutants: Q61R, Q61K, G12D, Q61L, Q61H, G13R, G13D, G12S, G12C, G12V, G12A, G13V, G12R, P185S, G13C, A146T, G60E, Q61P, A59D, E132K, E49K, T501, A146V, or A59T, and combinations thereof;
   or a combination of any of the foregoing. In some embodiments, the cancer comprises a K-Ras mutation selected from the group consisting of G12C, G12D, G13C, G12V, G13D, G12R, G12S, Q61H, Q61K and Q61L. In some embodiments, the cancer comprises an N-Ras mutation selected from the group consisting of G12C, Q61H, Q61K, Q61L, Q61P and Q61R. In some embodiments, the cancer comprises an H-Ras mutation selected from the group consisting of Q61H and Q61L. In some embodiments, the cancer comprises a Ras mutation selected from the group consisting of G12C, G13C, G12A, G12D, G13D, G12S, G13S, G12V and G13V. In some embodiments, the cancer comprises at least two Ras mutations selected from the group consisting of G12C, G13C, G12A, G12D, G13D, G12S, G13S, G12V and G13V. In some embodiments, a compound of the present invention inhibits more than one Ras mutant. For example, a compound may inhibit both K-Ras G12C and K-Ras G13C. A compound may inhibit both N-Ras G12C and K-Ras G12C. In some embodiments, a compound may inhibit both K-Ras G12C and K-Ras G12D. In some embodiments, a compound may inhibit both K-Ras G12V and K-Ras G12C. In some embodiments, a compound may inhibit both K-Ras G12V and K-Ras G12S. In some embodiments, a compound of the present invention inhibits Ras^{WT} in addition to one or more additional Ras mutations (e.g., K-, H- or N-Ras^{WT} and K-Ras G12D, G12V, G12C, G13D, G12R, G12A, Q61H, G12S, A146T, G13C, Q61L, Q61R, K117N, A146V, G12F, Q61K, L19F, Q22K, V141, A59T, A146P, G13R, G12L, or G13V; K, H or N-Ras^{WT} and H-Ras Q61R, G13R, Q61K, G12S, Q61L, G12D, G13V, G13D, G12C, K117N, A59T, G12V, G13C, Q61H, G13S, A18V, D119N, G13N, A146T, A66T, G12A, A146V, G12N, or G12R; or K, H or N-Ras^{WT} and N-Ras Q61R, Q61K, G12D, Q61L, Q61H, G13R, G13D, G12S, G12C, G12V, G12A, G13V, G12R, P185S, G13C, A146T, G60E, Q61P, A59D, E132K, E49K, T501, A146V, or A59T). In some embodiments, a compound of the present invention inhibits Ras^{amp} in addition to one or more additional Ras mutations (e.g., K-, H- or N-Ras^{amp} and K-Ras G12D, G12V, G12C, G13D, G12R, G12A, Q61H, G12S, A146T, G13C, Q61L, Q61R, K117N, A146V, G12F, Q61K, L19F, Q22K, V141, A59T, A146P, G13R, G12L, or G13V; K, H or N-Ras^{amp} and H-Ras Q61R, G13R, Q61K, G12S, Q61L, G12D, G13V, G13D, G12C, K117N, A59T, G12V, G13C, Q61H, G13S, A18V, D119N, G13N, A146T, A66T, G12A, A146V, G12N, or G12R; or K, H or N-Ras^{amp} and N-Ras Q61R, Q61K, G12D, Q61L, Q61H, G13R, G13D, G12S, G12C, G12V, G12A, G13V, G12R, P185S, G13C, A146T, G60E, Q61P, A59D, E132K, E49K, T501, A146V, or A59T).

Methods of detecting Ras mutations are known in the art. Such means include, but are not limited to direct sequencing, and utilization of a high-sensitivity diagnostic assay (with CE-IVD mark), e.g., as described in Domagala, et al., Pol J Pathol 3:145-164 (2012), incorporated herein by reference in its entirety, including TheraScreen PCR; AmoyDx; PNAClamp; RealQuality; EntroGen; LightMix; StripAssay; Hybcell plexA; Devyser; Surveyor; Cobas; and TheraScreen Pyro. See, also, e.g., WO 2020/106640.

In some embodiments, the cancer is non-small cell lung cancer, and the Ras mutation comprises a K-Ras mutation, such as K-Ras G12C, K-Ras G12V or K-Ras G12D. In some embodiments, the cancer is colorectal cancer, and the Ras mutation comprises a K-Ras mutation, such as K-Ras G12C, K-Ras G12V or K-Ras G12D. In some embodiments, the cancer is pancreatic cancer, and the Ras mutation comprises a K-Ras mutation, such as K-Ras G12D or K-Ras G12V. In some embodiments, the cancer is pancreatic cancer, and the Ras mutation comprises an N-Ras mutation, such as N-Ras G12D. In some embodiments, the cancer is melanoma, and the Ras mutation comprises an N-Ras mutation, such as N-Ras Q61R or N-Ras Q61K. In some embodiments, the cancer is non-small cell lung cancer, and the Ras protein is K-Ras^{amp}. In any of the foregoing if not already specified, a compound may inhibit Ras^{WT} (e.g., K-, H- or N-Ras^{WT}) or Ras^{amp} (e.g., K-, H- or N-Ras^{amp}) as well.

In some embodiments, a cancer comprises a Ras mutation and an STK11^{LOF}, a KEAP1, an EPHA5 or an NF1 mutation. In some embodiments, the cancer is non-small cell lung cancer and comprises a K-Ras G12C mutation. In some embodiments, the cancer is non-small cell lung cancer and comprises a K-Ras G12C mutation and an STK11^{LOF} mutation. In some embodiments, the cancer is non-small cell lung cancer and comprises a K-Ras G12C mutation and an STK11^{LOF} mutation. In some embodiments, a cancer comprises a K-Ras G13C Ras mutation and an STK11^{LOF}, a KEAP1, an EPHA5 or an NF1 mutation. In some embodiments, the cancer is non-small cell lung cancer and comprises a K-Ras G12D mutation. In some embodiments, the cancer is non-small cell lung cancer and comprises a K-Ras G12V mutation. In some embodiments, the cancer is colorectal cancer and comprises a K-Ras G12C mutation. In some embodiments, the cancer is pancreatic cancer and comprises a K-Ras G12C or K-Ras G12D mutation. In some embodiments, the cancer is pancreatic cancer and comprises a a K-Ras G12V mutation. In some embodiments, the cancer is endometrial cancer, ovarian cancer, cholangiocarcinoma, or mucinous appendiceal cancer and comprises a K-Ras G12C mutation. In some embodiments, the cancer is gastric cancer and comprises a K-Ras G12C mutation. In any of the foregoing, a compound may inhibit Ras^{WT} (e.g., K-, H- or N-Ras^{WT}) or Ras^{amp} (e.g., K-, H- or N-Ras^{amp}) as well.

Also provided is a method of inhibiting a Ras protein in a cell, the method comprising contacting the cell with an effective amount of a compound of the present invention, or a pharmaceutically acceptable salt thereof. A method of inhibiting RAF-Ras binding, the method comprising contacting the cell with an effective amount of a compound of the present invention, or a pharmaceutically acceptable salt thereof, is also provided. The cell may be a cancer cell. The cancer cell may be of any type of cancer described herein. The cell may be in vivo or in vitro.

### Combination Therapy

The methods of the invention may include a compound of the invention used alone or in combination with one or more additional therapies (e.g., non-drug treatments or therapeutic agents). The dosages of one or more of the additional therapies (e.g., non-drug treatments or therapeutic agents) may be reduced from standard dosages when administered alone. For example, doses may be determined empirically from drug combinations and permutations or may be deduced by isobolographic analysis (e.g., Black et al., Neurology 65:S3-S6(2005)).

A compound of the present invention may be administered before, after, or concurrently with one or more of such additional therapies. When combined, dosages of a compound of the invention and dosages of the one or more additional therapies (e.g., non-drug treatment or therapeutic agent) provide a therapeutic effect (e.g., synergistic or additive therapeutic effect). A compound of the present invention and an additional therapy, such as an anti-cancer agent, may be administered together, such as in a unitary pharmaceutical composition, or separately and, when administered separately, this may occur simultaneously or sequentially. Such sequential administration may be close or remote in time.

In some embodiments, the additional therapy is the administration of side-effect limiting agents (e.g., agents intended to lessen the occurrence or severity of side effects of treatment. For example, in some embodiments, the compounds of the present invention can also be used in combination with a therapeutic agent that treats nausea. Examples of agents that can be used to treat nausea include: dronabinol, granisetron, metoclopramide, ondansetron, and prochlorperazine, or pharmaceutically acceptable salts thereof.

In some embodiments, the one or more additional therapies includes a non-drug treatment (e.g., surgery or radiation therapy). In some embodiments, the one or more additional therapies includes a therapeutic agent (e.g., a compound or biologic that is an anti-angiogenic agent, signal transduction inhibitor, antiproliferative agent, glycolysis inhibitor, or autophagy inhibitor). In some embodiments, the one or more additional therapies includes a non-drug treatment (e.g., surgery or radiation therapy) and a therapeutic agent (e.g., a compound or biologic that is an anti-angiogenic agent, signal transduction inhibitor, antiproliferative agent, glycolysis inhibitor, or autophagy inhibitor). In other embodiments, the one or more additional therapies includes two therapeutic agents. In still other embodiments, the one or more additional therapies includes three therapeutic agents. In some embodiments, the one or more additional therapies includes four or more therapeutic agents.

In this Combination Therapy section, all references are incorporated by reference for the agents described, whether explicitly stated as such or not.

### Non-Drug Therapies

Examples of non-drug treatments include, but are not limited to, radiation therapy, cryotherapy, hyperthermia, surgery (e.g., surgical excision of tumor tissue), and T cell adoptive transfer (ACT) therapy. In some embodiments, the compounds of the invention may be used as an adjuvant therapy after surgery. In some embodiments, the compounds of the invention may be used as a neo-adjuvant therapy prior to surgery.

Radiation therapy may be used for inhibiting abnormal cell growth or treating a hyperproliferative disorder, such as cancer, in a subject (e.g., mammal (e.g., human)). Techniques for administering radiation therapy are known in the art. Radiation therapy can be administered through one of several methods, or a combination of methods, including, without limitation, external-beam therapy, internal radiation therapy, implant radiation, stereotactic radiosurgery, systemic radiation therapy, radiotherapy, and permanent or temporary interstitial brachy therapy. The term “brachy therapy.” as used herein, refers to radiation therapy delivered by a spatially confined radioactive material inserted into the body at or near a tumor or other proliferative tissue disease site. The term is intended, without limitation, to include exposure to radioactive isotopes (e.g., At-211, I-131, I-125, Y-90, Re-186, Re-188, Sm-153, Bi-212, P-32, and radioactive isotopes of Lu). Suitable radiation sources for use as a cell conditioner of the present invention include both solids and liquids. By way of non-limiting example, the radiation source can be a radionuclide, such as I-125, I-131, Yb-169, Ir-192 as a solid source, 1-125 as a solid source, or other radionuclides that emit photons, beta particles, gamma radiation, or other therapeutic rays. The radioactive material can also be a fluid made from any solution of radionuclide(s), e.g., a solution of I-125 or I-131, or a radioactive fluid can be produced using a slurry of a suitable fluid containing small particles of solid radionuclides, such as Au-198, or Y-90. Moreover, the radionuclide(s) can be embodied in a gel or radioactive micro spheres.

In some embodiments, the compounds of the present invention can render abnormal cells more sensitive to treatment with radiation for purposes of killing or inhibiting the growth of such cells. Accordingly, this invention further relates to a method for sensitizing abnormal cells in a mammal to treatment with radiation which comprises administering to the mammal an amount of a compound of the present invention, which amount is effective to sensitize abnormal cells to treatment with radiation. The amount of the compound in this method can be determined according to the means for ascertaining effective amounts of such compounds described herein. In some embodiments, the compounds of the present invention may be used as an adjuvant therapy after radiation therapy or as a neo-adjuvant therapy prior to radiation therapy.

In some embodiments, the non-drug treatment is a T cell adoptive transfer (ACT) therapy. In some embodiments, the T cell is an activated T cell. The T cell may be modified to express a chimeric antigen receptor (CAR). CAR modified T (CAR-T) cells can be generated by any method known in the art. For example, the CAR-T cells can be generated by introducing a suitable expression vector encoding the CAR to a T cell. Prior to expansion and genetic modification of the T cells, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available in the art may be used. In some embodiments, the T cell is an autologous T cell. Whether prior to or after genetic modification of the T cells to express a desirable protein (e.g., a CAR), the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 7,572,631; 5,883,223; 6,905,874; 6,797,514; and 6,867,041.

### Therapeutic Agents

A therapeutic agent may be a compound used in the treatment of cancer or symptoms associated therewith. A compound of the present invention may be combined with a second, third, or fourth therapeutic agent, or more. A compound of the present invention may be combined with one or more therapeutic agents along with one or more non-drug therapies.

For example, a therapeutic agent may be a steroid. Steroids are known in the art. Accordingly, in some embodiments, the one or more additional therapies includes a steroid. Suitable steroids may include, but are not limited to, 21-acetoxypregnenolone, alclometasone, algestone, amcinonide, beclomethasone, betamethasone, budesonide, chloroprednisone, clobetasol, clocortolone, cloprednol, corticosterone, cortisone, cortivazol, deflazacort, desonide, desoximetasone, dexamethasone, diflorasone, diflucortolone, difuprednate, enoxolone, fluazacort, fiucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, hydrocortisone, loteprednol etabonate, mazipredone, medrysone, meprednisone, methylprednisolone, mometasone furoate, paramethasone, prednicarbate, prednisolone, prednisolone 25-diethylaminoacetate, prednisolone sodium phosphate, prednisone, prednival, prednylidene, rimexolone, tixocortol, triamcinolone, triamcinolone acetonide, triamcinolone benetonide, triamcinolone hexacetonide, and salts or derivatives thereof.

Further examples of therapeutic agents that may be used in combination therapy with a compound of the present invention include compounds described in the following patents: U.S. Pat. Nos. 6,258,812, 6,630,500, 6,515,004, 6,713,485, 5,521,184, 5,770,599, 5,747,498, 5,990,141, 6,235,764, and 8,623,885, and International Patent Applications WO01/37820, WO01/32651, WO02/68406, WO02/66470, WO02/55501, WO04/05279, WO04/07481, WO04/07458, WO04/09784, WO02/59110, WO99/45009, WO00/59509, WO99/61422, WO00/12089, and WO00/02871.

A therapeutic agent may be a biologic (e.g., cytokine (e.g., interferon or an interleukin such as IL-2)) used in treatment of cancer or symptoms associated therewith. Biologics are known in the art. In some embodiments, the biologic is an immunoglobulin-based biologic, e.g., a monoclonal antibody (e.g., a humanized antibody, a fully human antibody, an Fc fusion protein, or a functional fragment thereof) that agonizes a target to stimulate an anti-cancer response or antagonizes an antigen important for cancer. Also included are antibody-drug conjugates.

A therapeutic agent may be a T-cell checkpoint inhibitor. Such checkpoint inhibitors are known in the art. In one embodiment, the checkpoint inhibitor is an inhibitory antibody (e.g., a monospecific antibody such as a monoclonal antibody). The antibody may be, e.g., humanized or fully human. In some embodiments, the checkpoint inhibitor is a fusion protein, e.g., an Fc-receptor fusion protein. In some embodiments, the checkpoint inhibitor is an agent, such as an antibody, that interacts with a checkpoint protein. In some embodiments, the checkpoint inhibitor is an agent, such as an antibody, that interacts with the ligand of a checkpoint protein. In some embodiments, the checkpoint inhibitor is an inhibitor (e.g., an inhibitory antibody or small molecule inhibitor) of CTLA-4 (e.g., an anti-CTLA-4 antibody or fusion a protein). In some embodiments, the checkpoint inhibitor is an inhibitor or antagonist (e.g., an inhibitory antibody or small molecule inhibitor) of PD-1. In some embodiments, the checkpoint inhibitor is an inhibitor or antagonist (e.g., an inhibitory antibody or small molecule inhibitor) of PD-L1. In some embodiments, the checkpoint inhibitor is an inhibitor or antagonist (e.g., an inhibitory antibody or Fc fusion or small molecule inhibitor) of PD-L2 (e.g., a PD-L2/lg fusion protein). In some embodiments, the checkpoint inhibitor is an inhibitor or antagonist (e.g., an inhibitory antibody or small molecule inhibitor) of B7-H3, B7-H4, BTLA, HVEM, TIM3, GAL9, LAG3, VISTA, KIR, 2B4, CD160, CGEN-15049, CHK 1, CHK2, A2aR, B-7 family ligands, or a combination thereof. In some embodiments, the checkpoint inhibitor is pembrolizumab, nivolumab, PDR001 (NVS), REGN2810 (Sanofi/Regeneron), a PD-L1 antibody such as, e.g., avelumab, durvalumab, atezolizumab, pidilizumab, JNJ-63723283 (JNJ), BGB-A317 (BeiGene & Celgene) or a checkpoint inhibitor disclosed in Preusser, M. et al. (2015) Nat. Rev. Neurol., including, without limitation, ipilimumab, tremelimumab, nivolumab, pembrolizumab, AMP224, AMP514/MEDI0680, BMS936559, MEDI4736, MPDL3280A, MSB0010718C, BMS986016, IMP321, lirilumab, IPH2101, 1-7F9, and KW-6002.

A therapeutic agent may be an anti-TIGIT antibody, such as MBSA43, BMS-986207, MK-7684, COM902, AB154, MTIG7192A or OMP-313M32 (etigilimab). Other anti-TIGIT antibodies are known in the art.

A therapeutic agent may be an agent that treats cancer or symptoms associated therewith (e.g., a cytotoxic agent, non-peptide small molecules, or other compound useful in the treatment of cancer or symptoms associated therewith, collectively, an “anti-cancer agent”). Anti-cancer agents can be, e.g., chemotherapeutics or targeted therapy agents. Such agents are known in the art.

Anti-cancer agents include mitotic inhibitors, intercalating antibiotics, growth factor inhibitors, cell cycle inhibitors, enzymes, topoisomerase inhibitors, biological response modifiers, alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Further anti-cancer agents include leucovorin (LV), irenotecan, oxaliplatin, capecitabine, paclitaxel, and doxetaxel. In some embodiments, the one or more additional therapies includes two or more anti-cancer agents. The two or more anti-cancer agents can be used in a cocktail to be administered in combination or administered separately. Suitable dosing regimens of combination anti-cancer agents are known in the art and described in, for example, Saltz et al., Proc. Am. Soc. Clin. Oncol. 18: 233a (1999), and Douillard et al., Lancet 355 (9209): 1041-1047 (2000).

Other non-limiting examples of anti-cancer agents include Gleevec® (Imatinib Mesylate); Kyprolis® (carfilzomib); Velcade® (bortezomib); Casodex (bicalutamide); Iressa® (gefitinib); alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; sarcodictyin A; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimustine; antibiotics such as the enediyne antibiotics (e.g., calicheamicin, such as calicheamicin gammall and calicheamicin omegall (see, e.g., Agnew, Chem. Intl. Ed Engl. 33:183-186 (1994)); dynemicin such as dynemicin A; bisphosphonates such as clodronate; an esperamicin; neocarzinostatin chromophore and related chromoprotein enediyne antiobiotic chromophores, aclacinomysins, actinomycin, authramycin, azaserine, bleomycins, cactinomycin, calicheamicin, carabicin, caminomycin, carminomycin, carzinophilin, chromomycins, dactinomycin, daunorubicin, detorubicin, 6-diazo-5-oxo-L-norleucine, adriamycin (doxorubicin), morpholino-doxorubicin, cyanomorpholino-doxorubicin, 2-pyrrolino-doxorubicin, deoxydoxorubicin, epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenishers such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone such as epothilone B; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofiran; spirogermanium; tenuazonic acid; triaziquone; 2,2′,2″-trichlorotriethylamine; trichothecenes such as T-2 toxin, verracurin A, roridin A and anguidine; urethane; vindesine; dacarbazine; mannomustine; mitobronitol; mitolactol; pipobroman; gacytosine; arabinoside (“Ara-C”); cyclophosphamide; thiotepa; taxoids, e.g., Taxol® (paclitaxel), Abraxane® (cremophor-free, albumin-engineered nanoparticle formulation of paclitaxel), and Taxotere® (doxetaxel); chloranbucil; tamoxifen (Nolvadex™); raloxifene; aromatase inhibiting 4 (5)-imidazoles; 4-hydroxytamoxifen; trioxifene; keoxifene; LY 117018; onapristone; toremifene (Fareston®); flutamide, nilutamide, bicalutamide, leuprolide, goserelin; chlorambucil; Gemzar® gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; Navelbine® (vinorelbine); novantrone; teniposide; edatrexate; daunomycin; aminopterin; ibandronate; irinotecan (e.g., CPT-11); topoisomerase inhibitor RFS 2000; difluoromethylornithine (DMFO); retinoids such as retinoic acid; esperamicins; capecitabine (e.g., Xeloda®); and pharmaceutically acceptable salts of any of the above.

Additional non-limiting examples of anti-cancer agents include trastuzumab (Herceptin®), bevacizumab (Avastin®), cetuximab (Erbitux®), rituximab (Rituxan®), Taxol®, Arimidex®, ABVD, avicine, abagovomab, acridine carboxamide, adecatumumab, 17-N-allylamino-17-demethoxygeldanamycin, alpharadin, alvocidib, 3-aminopyridine-2-carboxaldehyde thiosemicarbazone, amonafide, anthracenedione, anti-CD22 immunotoxins, antineoplastics (e.g., cell-cycle nonspecific antineoplastic agents, and other antineoplastics described herein), antitumorigenic herbs, apaziquone, atiprimod, azathioprine, belotecan, bendamustine, BIBW 2992, biricodar, brostallicin, bryostatin, buthionine sulfoximine, CBV (chemotherapy), calyculin, dichloroacetic acid, discodermolide, elsamitrucin, enocitabine, eribulin, exatecan, exisulind, ferruginol, forodesine, fosfestrol, ICE chemotherapy regimen, IT-101, imexon, imiquimod, indolocarbazole, irofulven, laniquidar, larotaxel, lenalidomide, lucanthone, lurtotecan, mafosfamide, mitozolomide, nafoxidine, nedaplatin, olaparib, ortataxel, PAC-1, pawpaw, pixantrone, proteasome inhibitors, rebeccamycin, resiquimod, rubitecan, SN-38, salinosporamide A, sapacitabine, Stanford V, swainsonine, talaporfin, tariquidar, tegafur-uracil, temodar, tesetaxel, triplatin tetranitrate, tris (2-chloroethyl) amine, troxacitabine, uramustine, vadimezan, vinflunine, ZD6126, and zosuquidar.

Further non-limiting examples of anti-cancer agents include natural products such as vinca alkaloids (e.g., vinblastine, vincristine, and vinorelbine), epidipodophyllotoxins (e.g., etoposide and teniposide), antibiotics (e.g., dactinomycin (actinomycin D), daunorubicin, and idarubicin), anthracyclines, mitoxantrone, bleomycins, plicamycin (mithramycin), mitomycin, enzymes (e.g., L-asparaginase which systemically metabolizes L-asparagine and deprives cells which do not have the capacity to synthesize their own asparagine), antiplatelet agents, antiproliferative/antimitotic alkylating agents such as nitrogen mustards (e.g., mechlorethamine, cyclophosphamide and analogs, melphalan, and chlorambucil), ethylenimines and methylmelamines (e.g., hexaamethylmelaamine and thiotepa), CDK inhibitors (e.g., a CDK4/6 inhibitor such as abemaciclib, ribociclib, palbociclib; seliciclib, UCN-01, P1446A-05, PD-0332991, dinaciclib, P27-00, AT-7519, RGB286638, and SCH727965), alkyl sulfonates (e.g., busulfan), nitrosoureas (e.g., carmustine (BCNU) and analogs, and streptozocin), trazenes-dacarbazinine (DTIC), antiproliferative/antimitotic antimetabolites such as folic acid analogs, pyrimidine analogs (e.g., fluorouracil, floxuridine, and cytarabine), purine analogs and related inhibitors (e.g., mercaptopurine, thioguanine, pentostatin, and 2-chlorodeoxyadenosine), aromatase inhibitors (e.g., anastrozole, exemestane, and letrozole), and platinum coordination complexes (e.g., cisplatin and carboplatin), procarbazine, hydroxyurea, mitotane, aminoglutethimide, histone deacetylase (HDAC) inhibitors (e.g., trichostatin, sodium butyrate, apicidan, suberoyl anilide hydroamic acid, vorinostat, belinostat, LBH 589, romidepsin, ACY-1215, and panobinostat), mTOR inhibitors (e.g., vistusertib, temsirolimus, everolimus, ridaforolimus, and sirolimus), KSP (Eg5) inhibitors (e.g., Array 520), DNA binding agents (e.g., Zalypsis®), PI3K inhibitors such as PI3K delta inhibitor (e.g., GS-1101 and TGR-1202), PI3K delta and gamma inhibitor (e.g., CAL-130), copanlisib, alpelisib and idelalisib; multi-kinase inhibitor (e.g., TG02 and sorafenib), hormones (e.g., estrogen) and hormone agonists such as leutinizing hormone releasing hormone (LHRH) agonists (e.g., goserelin, leuprolide and triptorelin), BAFF-neutralizing antibody (e.g., LY2127399), IKK inhibitors, p38MAPK inhibitors, anti-IL-6 (e.g., CNT0328), telomerase inhibitors (e.g., GRN 163 L), aurora kinase inhibitors (e.g., MLN8237), cell surface monoclonal antibodies (e.g., anti-CD38 (HUMAX-CD38), anti-CSI (e.g., elotuzumab), HSP90 inhibitors (e.g., 17 AAG and KOS 953), P13K/Akt inhibitors (e.g., perifosine), Akt inhibitors (e.g., GSK-2141795), PKC inhibitors (e.g., enzastaurin), FTIs (e.g., Zarnestra™), anti-CD138 (e.g., BT062), Torcl/2 specific kinase inhibitors (e.g., INK128), ER/UPR targeting agents (e.g., MKC-3946), cFMS inhibitors (e.g., ARRY-382), JAK1/2 inhibitors (e.g., CYT387), PARP inhibitors (e.g., olaparib and veliparib (ABT-888)), and BCL-2 antagonists.

In some embodiments, an anti-cancer agent is selected from mechlorethamine, camptothecin, ifosfamide, tamoxifen, raloxifene, gemcitabine, Navelbine®, sorafenib, or any analog or derivative variant of the foregoing.

In some embodiments, the anti-cancer agent is a HER2 inhibitor. HER2 inhibitors are known in the art. Non-limiting examples of HER2 inhibitors include monoclonal antibodies such as trastuzumab (Herceptin®) and pertuzumab (Perjeta®); small molecule tyrosine kinase inhibitors such as gefitinib (Iressa®), erlotinib (Tarceva®), pilitinib, CP-654577, CP-724714, canertinib (CI 1033), HKI-272, lapatinib (GW-572016; Tykerb®), PKI-166, AEE788, BMS-599626, HKI-357, BIBW 2992, ARRY-334543, and JNJ-26483327.

In some embodiments, an anti-cancer agent is an ALK inhibitor. ALK inhibitors are known in the art. Non-limiting examples of ALK inhibitors include ceritinib, TAE-684 (NVP-TAE694), PF02341066 (crizotinib or 1066), alectinib; brigatinib; entrectinib; ensartinib (X-396); lorlatinib; ASP3026; CEP-37440; 4SC-203; TL-398; PLB1003; TSR-011; CT-707; TPX-0005, and AP26113. Additional examples of ALK kinase inhibitors are described in examples 3-39 of WO05016894.

In some embodiments, an anti-cancer agent is an inhibitor of a member downstream of a Receptor Tyrosine Kinase (RTK)/Growth Factor Receptor (e.g., a SHP2 inhibitor (e.g., SHP099, TNO155, RMC-4550, RMC-4630, JAB-3068, JAB-3312, RLY-1971, ERAS-601, SH3809, PF-07284892, or BBP-398), or a pharmaceutically acceptable salt, solvate, isomer (e.g., stereoisomer), prodrug, or tautomer thereof), an SOS1 inhibitor (e.g., BI-1701963, BI-3406, SDR5, BAY-293 or RMC-5845, or a pharmaceutically acceptable salt, solvate, isomer (e.g., stereoisomer), prodrug, or tautomer thereof), a Raf inhibitor, a MEK inhibitor, an ERK inhibitor, a PI3K inhibitor, a PTEN inhibitor, an AKT inhibitor, or an mTOR inhibitor (e.g., mTORC1 inhibitor or mTORC2 inhibitor). In some embodiments, the anti-cancer agent is JAB-3312.

In some embodiments, an anti-cancer agent is a SOS1 inhibitor. SOS1 inhibitors are known in the art. In some embodiments, the SOS1 inhibitor is selected from those disclosed in WO 2022146698, WO 2022081912, WO 2022058344, WO 2022026465, WO 2022017519, WO 2021173524, WO 2021130731, WO 2021127429, WO 2021092115, WO 2021105960, WO 2021074227, WO 2020180768, WO 2020180770, WO 2020173935, WO 2020146470, WO 2019201848, WO 2019122129, WO 2018172250, and WO 2018115380, or a pharmaceutically acceptable salt, solvate, isomer (e.g., stereoisomer), prodrug, or tautomer thereof. In some embodiments, a compound of the present invention is used in combination with a SOS1 inhibitor to treat a K-Ras G13C cancer.

In some embodiments, an anti-cancer agent is an additional Ras inhibitor or a Ras vaccine, or another therapeutic modality designed to directly or indirectly decrease the oncogenic activity of Ras. Such agents are known in the art. In some embodiments, an anti-cancer agent is an additional Ras inhibitor. In some embodiments, the Ras inhibitor targets Ras in its active, or GTP-bound state. In some embodiments, the Ras inhibitor targets Ras in its inactive, or GDP-bound state. In some embodiments, the Ras inhibitor is, such as an inhibitor of K-Ras G12C, such as AMG 510, MRTX1257, MRTX849, JNJ-74699157, LY3499446, or ARS-1620, ARS-853, BPI-421286, LY3537982, JDQ443, JAB-21822, JAB-21000, IBI351, ERAS-3490, RMC-6291, or GDC-6036. In some embodiments, the Ras inhibitor is an inhibitor of K-Ras G12D, such as MRTX1133 or JAB-22000. In some embodiments, the Ras inhibitor is a K-Ras G12V inhibitor, such as JAB-23000. In some embodiments, the Ras inhibitor is RMC-6236. In some embodiments, the Ras inhibitor is selected from a Ras (ON) inhibitor (that is, Ras in its GTP-bound state) disclosed in the following, incorporated herein by reference in their entireties, or a pharmaceutically acceptable salt, solvate, isomer (e.g., stereoisomer), prodrug, or tautomer thereof: WO 2021091982, WO 2021091967, WO 2021091956, and WO 2020132597. Other examples of Ras inhibitors are known in the art, such as in the following, incorporated herein by reference in their entireties: WO 2022271658, WO 2022269508, WO 2022266167, WO 2022266069, WO 2022266015, WO 2022265974, WO 2022261154, WO 2022261154, WO 2022251576, WO 2022251296, WO 2022237815, WO 2022232332, WO 2022232331, WO 2022232320, WO 2022232318, WO 2022223037, WO 2022221739, WO 2022221528, WO 2022221386, WO 2022216762, WO 2022192794, WO 2022192790, WO 2022188729, WO 2022187411, WO 2022184178, WO 2022173870, WO 2022173678, WO 2022135346, WO 2022133731, WO 2022133038, WO 2022133345, WO 2022132200, WO 2022119748, WO 2022109485, WO 2022109487, WO 2022066805, WO 2022002102, WO 2022002018, WO 2021259331, WO 2021257828, WO 2021252339, WO 2021248095, WO 2021248090, WO 2021248083, WO 2021248082, WO 2021248079, WO 2021248055, WO 2021245051, WO 2021244603, WO 2021239058, WO 2021231526, WO 2021228161, WO 2021219090, WO 2021219090, WO 2021219072, WO 2021218939, WO 2021217019, WO 2021216770, WO 2021215545, WO 2021215544, WO 2021211864, WO 2021190467, WO 2021185233, WO 2021180181, WO 2021175199, 2021173923, WO 2021169990, WO 2021169963, WO 2021168193, WO 2021158071, WO 2021155716, WO 2021152149, WO 2021150613, WO 2021147967, WO 2021147965, WO 2021143693, WO 2021142252, WO 2021141628, WO 2021139748, WO 2021139678, WO 2021129824, WO 2021129820, WO 2021127404, WO 2021126816, WO 2021126799, WO 2021124222, WO 2021121371, WO 2021121367, WO 2021121330, WO 2020050890, WO 2020047192, WO 2020035031, WO 2020028706, WO 2019241157, WO 2019232419, WO 2019217691, WO 2019217307, WO 2019215203, WO 2019213526, WO 2019213516, WO 2019155399, WO 2019150305, WO 2019110751, WO 2019099524, WO 2019051291, WO 2018218070, WO 2018217651, WO 2018218071, WO 2018218069, WO 2018206539, WO 2018143315, WO 2018140600, WO 2018140599, WO 2018140598, WO 2018140514, WO 2018140513, WO 2018140512, WO 2018119183, WO 2018112420, WO 2018068017, WO 2018064510, WO 2017201161, WO 2017172979, WO 2017100546, WO 2017087528, WO 2017058807, WO 2017058805, WO 2017058728, WO 2017058902, WO 2017058792, WO 2017058768, WO 2017058915, WO 2017015562, WO 2016168540, WO 2016164675, WO 2016049568, WO 2016049524, WO 2015054572, WO 2014152588, WO 2014143659 and WO 2013155223.

In some embodiments, a therapeutic agent that may be combined with a compound of the present invention is an inhibitor of the MAP kinase (MAPK) pathway (or “MAPK inhibitor”). Such agents are known in the art. MAPK inhibitors include, but are not limited to, one or more MAPK inhibitor described in Cancers (Basel) 2015 September; 7 (3): 1758-1784. For example, the MAPK inhibitor may be selected from one or more of trametinib, binimetinib, selumetinib, cobimetinib, LErafAON (NeoPharm), ISIS 5132; vemurafenib, pimasertib, TAK733, RO4987655 (CH4987655); CI-1040; PD-0325901; CH5126766; MAP855; AZD6244; refametinib (RDEA 119/BAY 86-9766); GDC-0973/XL581; AZD8330 (ARRY-424704/ARRY-704); RO5126766 (Roche, described in PLOS One. 2014 Nov. 25; 9 (11)); and GSK1120212 (or JTP-74057, described in Clin Cancer Res. 2011 Mar. 1; 17 (5):989-1000). The MAPK inhibitor may be PLX8394, LXH254, GDC-5573, or LY3009120.

In some embodiments, an anti-cancer agent is a disrupter or inhibitor of the RAS-RAF-ERK or PI3K-AKT-TOR or PI3K-AKT signaling pathways. Such agents are known in the art. The PI3K/AKT inhibitor may include, but is not limited to, one or more PI3K/AKT inhibitor described in Cancers (Basel) 2015 September; 7(3): 1758-1784. For example, the PI3K/AKT inhibitor may be selected from one or more of NVP-BEZ235; BGT226; XL765/SAR245409; SF1126; GDC-0980; PI-103; PF-04691502; PKI-587; GSK2126458.

In some embodiments, an anti-cancer agent is a PD-1 or PD-L1 antagonist. Such agents are known in the art.

In some embodiments, additional therapeutic agents include ALK inhibitors, HER2 inhibitors, EGFR inhibitors, IGF-1R inhibitors, MEK inhibitors, PI3K inhibitors, AKT inhibitors, TOR inhibitors, MCL-1 inhibitors, BCL-2 inhibitors, SHP2 inhibitors, proteasome inhibitors, and immune therapies. In some embodiments, additional therapeutic agents include FGFR inhibitors, PARP inhibitors, BET inhibitors, PRMT5i inhibitors, MAT2A inhibitors, VEGF inhibitors, and HDAC inhibitors. In some embodiments, a therapeutic agent may be a pan-RTK inhibitor, such as afatinib.

IGF-1R inhibitors are known in the art and include linsitinib, or a pharmaceutically acceptable salt thereof.

EGFR inhibitors are known in the art and include, but are not limited to, small molecule antagonists, antibody inhibitors, or specific antisense nucleotide or siRNA. Useful antibody inhibitors of EGFR include cetuximab (Erbitux®), panitumumab (Vectibix®), zalutumumab, nimotuzumab, and matuzumab. Further antibody-based EGFR inhibitors include any anti-EGFR antibody or antibody fragment that can partially or completely block EGFR activation by its natural ligand. Non-limiting examples of antibody-based EGFR inhibitors include those described in Modjtahedi et al., Br. J. Cancer 1993, 67:247-253; Teramoto et al., Cancer 1996, 77:639-645; Goldstein et al., Clin. Cancer Res. 1995, 1:1311-1318; Huang et al., 1999, Cancer Res. 15: 59 (8): 1935-40; and Yang et al., Cancer Res. 1999, 59:1236-1243. The EGFR inhibitor can be monoclonal antibody Mab E7.6.3 (Yang, 1999 supra), or Mab C225 (ATCC Accession No. HB-8508), or an antibody or antibody fragment having the binding specificity thereof.

Small molecule antagonists of EGFR include gefitinib (Iressa®), erlotinib (Tarceva®), and lapatinib (TykerB®). See, e.g., Yan et al., Pharmacogenetics and Pharmacogenomics in Oncology Therapeutic Antibody Development, BioTechniques 2005, 39 (4): 565-8; and Paez et al., EGFR Mutations in Lung Cancer Correlation with Clinical Response to Gefitinib Therapy, Science 2004, 304 (5676): 1497-500. In some embodiments, the EGFR inhibitor is osimertinib (Tagrisso®). Further non-limiting examples of small molecule EGFR inhibitors include any of the EGFR inhibitors described in the following patent publications, and all pharmaceutically acceptable salts of such EGFR inhibitors: EP 0520722; EP 0566226; WO96/33980; U.S. Pat. No. 5,747,498; WO96/30347; EP 0787772; WO97/30034; WO97/30044; WO97/38994; WO97/49688; EP 837063; WO98/02434; WO97/38983; WO95/19774; WO95/19970; WO97/13771; WO98/02437; WO98/02438; WO97/32881; DE 19629652; WO98/33798; WO97/32880; WO97/32880; EP 682027; WO97/02266; WO97/27199; WO98/07726; WO97/34895; WO96/31510; WO98/14449; WO98/14450; WO98/14451; WO95/09847; WO97/19065; WO98/17662; U.S. Pat. Nos. 5,789,427; 5,650,415; 5,656,643; WO99/35146; WO99/35132; WO99/07701; and WO92/20642. Additional non-limiting examples of small molecule EGFR inhibitors include any of the EGFR inhibitors described in Traxler et al., Exp. Opin. Ther. Patents 1998, 8 (12): 1599-1625. In some embodiments, an EGFR inhibitor is an ERBB inhibitor. In humans, the ERBB family contains HER1 (EGFR, ERBB1), HER2 (NEU, ERBB2), HER3 (ERBB3), and HER (ERBB4).

MEK inhibitors are known in the art and include, but are not limited to, pimasertib, selumetinib, cobimetinib (Cotellic®), trametinib (Mekinist®), and binimetinib (Mektovi®). In some embodiments, a MEK inhibitor targets a MEK mutation that is a Class I MEK1 mutation selected from D67N; P124L; P124S; and L177V. In some embodiments, the MEK mutation is a Class II MEK1 mutation selected from ΔE51-Q58; ΔF53-Q58; E203K; L177M; C121S; F53L; K57E; Q56P; and K57N.

PI3K inhibitors are known in the art and include, but are not limited to, wortmannin; 17-hydroxywortmannin analogs described in WO06/044453; 4-[2-(1H-Indazol-4-yl)-6-[4-(methylsulfonyl) piperazin-1-yl]methyl]thieno[3,2-d]pyrimidin-4-yl]morpholine (also known as pictilisib or GDC-0941 and described in WO09/036082 and WO09/055730); 2-methyl-2-[4-[3-methyl-2-oxo-8-(quinolin-3-yl)-2,3-dihydroimidazo[4,5-c]quinolin-1-yl]phenyl]propionitrile (also known as BEZ 235 or NVP-BEZ 235, and described in WO06/122806); (S)—I-(4-((2-(2-aminopyrimidin-5-yl)-7-methyl-4-morpholinothieno[3,2-d]pyrimidin-6-yl)methyl) piperazin-1-yl)-2-hydroxypropan-1-one (described in WO08/070740); LY294002 (2-(4-morpholinyl)-8-phenyl-4H-I-benzopyran-4-one (available from Axon Medchem); PI 103 hydrochloride (3-[4-(4-morpholinylpyrido-[3′,2′:4,5] furo [3,2-d]pyrimidin-2-yl]phenol hydrochloride (available from Axon Medchem); PIK 75 (2-methyl-5-nitro-2-[(6-bromoimidazo[1,2-a]pyridin-3-yl)methylene]-1-methylhydrazide-benzenesulfonic acid, monohydrochloride) (available from Axon Medchem); PIK 90 (N-(7,8-dimethoxy-2,3-dihydro-imidazo[1,2-c]quinazolin-5-yl)-nicotinamide (available from Axon Medchem); AS-252424 (5-[I-[5-(4-fluoro-2-hydroxy-phenyl)-furan-2-yl]-meth-(Z)-ylidene]-thiazolidine-2,4-dione (available from Axon Medchem); TGX-221 (7-methyl-2-(4-morpholinyl)-9-[1-(phenylamino)ethyl]-4H-pyrido-[1,2-a]pyrirnidin-4-one (available from Axon Medchem); XL-765; and XL-147. Other PI3K inhibitors include demethoxyviridin, perifosine, CAL101, PX-866, BEZ235, SF1126, INK1117, IPI-145, BKM120, XL147, XL765, Palomid 529, GSK1059615, ZSTK474, PWT33597, IC87114, TGI 00-115, CAL263, PI-103, GNE-477, CUDC-907, and AEZS-136.

AKT inhibitors are known in the art and include, but are not limited to, Akt-1-1 (inhibits Aktl) (Barnett et al., Biochem. J. 2005, 385 (Pt. 2): 399-408); Akt-1-1,2 (inhibits Akl and 2) (Barnett et al., Biochem. J. 2005, 385 (Pt. 2): 399-408); API-59CJ-Ome (e.g., Jin et al., Br. J. Cancer 2004, 91:1808-12); 1-H-imidazo[4,5-c]pyridinyl compounds (e.g., WO 05/011700); indole-3-carbinol and derivatives thereof (e.g., U.S. Pat. No. 6,656,963; Sarkar and Li J Nutr. 2004, 134 (12 Suppl): 3493S-3498S); perifosine (e.g., interferes with Akt membrane localization; Dasmahapatra et al. Clin. Cancer Res. 2004, 10 (15): 5242-52); phosphatidylinositol ether lipid analogues (e.g., Gills and Dennis Expert. Opin. Investig. Drugs 2004, 13:787-97); and triciribine (TCN or API-2 or NCI identifier: NSC 154020; Yang et al., Cancer Res. 2004, 64:4394-9).

mTOR inhibitors are known in the art and include, but are not limited to, ATP-competitive mTORC1/mTORC2 inhibitors, e.g., PI-103, PP242, PP30; Torin 1; FKBP12 enhancers; 4H-1-benzopyran-4-one derivatives; and rapamycin (also known as sirolimus) and derivatives thereof, including: temsirolimus (Torisel®); everolimus (Afinitor®; WO94/09010); ridaforolimus (also known as deforolimus or AP23573); rapalogs, e.g., as disclosed in WO98/02441 and WO01/14387, e.g. AP23464 and AP23841; 40-(2-hydroxyethyl) rapamycin; 40-[3-hydroxy (hydroxymethyl)methylpropanoate]-rapamycin (also known as CC1779); 40-epi-(tetrazolyt)-rapamycin (also called ABT578); 32-deoxorapamycin; 16-pentynyloxy-32 (S)-dihydrorapanycin; derivatives disclosed in WO05/005434; derivatives disclosed in U.S. Pat. Nos. 5,258,389, 5,118,677, 5,118,678, 5,100,883, 5,151,413, 5,120,842, and 5,256,790, and in WO94/090101, WO92/05179, WO93/111130, WO94/02136, WO94/02485, WO95/14023, WO94/02136, WO95/16691, WO96/41807, WO96/41807, and WO2018204416; and phosphorus-containing rapamycin derivatives (e.g., WO05/016252). In some embodiments, the mTOR inhibitor is a bisteric inhibitor (see, e.g., WO2018204416, WO2019212990 and WO2019212991), such as RMC-5552, having the structure

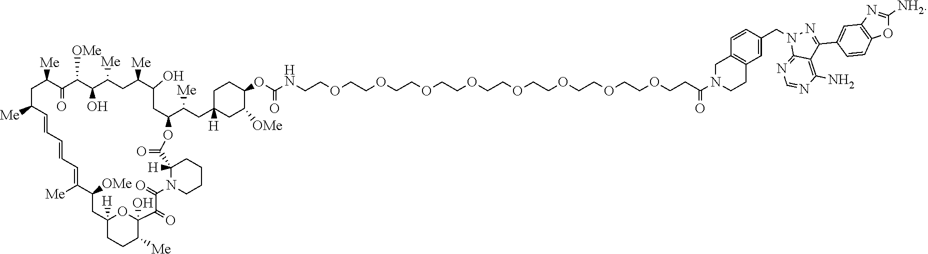 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)CC(O)[C@H](C)C[C@@H](C)[C@@H](O)[C@@H](OC)C(=O)[C@H](C)C[C@H](C)/C=C/C=C/C=C/1C]

BRAF inhibitors that may be used in combination with compounds of the invention are known in the art and include, for example, vemurafenib, dabrafenib, and encorafenib. A BRAF may comprise a Class 3 BRAF mutation. In some embodiments, the Class 3 BRAF mutation is selected from one or more of the following amino acid substitutions in human BRAF: D287H; P367R; V459L; G466V; G466E; G466A; S467L; G469E; N581S; N581I; D594N; D594G; D594A; D594H; F595L; G596D; G596R and A762E.

MCL-1 inhibitors are known in the art and include, but are not limited to, AMG-176, MIK665, and S63845. The myeloid cell leukemia-1 (MCL-1) protein is one of the key anti-apoptotic members of the B-cell lymphoma-2 (BCL-2) protein family. Over-expression of MCL-1 has been closely related to tumor progression as well as to resistance, not only to traditional chemotherapies but also to targeted therapeutics including BCL-2 inhibitors such as ABT-263.

In some embodiments, the additional therapeutic agent is a SHP2 inhibitor. SHP2 inhibitors are known in the art. SHP2 is a non-receptor protein tyrosine phosphatase encoded by the PTPN11 gene that contributes to multiple cellular functions including proliferation, differentiation, cell cycle maintenance and migration. SHP2 has two N-terminal Src homology 2 domains (N-SH2 and C-SH2), a catalytic domain (PTP), and a C-terminal tail. The two SH2 domains control the subcellular localization and functional regulation of SHP2. The molecule exists in an inactive, self-inhibited conformation stabilized by a binding network involving residues from both the N-SH2 and PTP domains. Stimulation by, for example, cytokines or growth factors acting through receptor tyrosine kinases (RTKs) leads to exposure of the catalytic site resulting in enzymatic activation of SHP2.

SHP2 is involved in signaling through the RAS-mitogen-activated protein kinase (MAPK), the JAK-STAT or the phosphoinositol 3-kinase-AKT pathways. Mutations in the PTPN11 gene and subsequently in SHP2 have been identified in several human developmental diseases, such as Noonan Syndrome and Leopard Syndrome, as well as human cancers, such as juvenile myelomonocytic leukemia, neuroblastoma, melanoma, acute myeloid leukemia and cancers of the breast, lung, and colon. Some of these mutations destabilize the auto-inhibited conformation of SHP2 and promote autoactivation or enhanced growth factor driven activation of SHP2. SHP2, therefore, represents a highly attractive target for the development of novel therapies for the treatment of various diseases including cancer. A SHP2 inhibitor (e.g., RMC-4550 or SHP099) in combination with a RAS pathway inhibitor (e.g., a MEK inhibitor) have been shown to inhibit the proliferation of multiple cancer cell lines in vitro (e.g., pancreas, lung, ovarian and breast cancer). Thus, combination therapy involving a SHP2 inhibitor with a RAS pathway inhibitor could be a general strategy for preventing tumor resistance in a wide range of malignancies.

Non-limiting examples of such SHP2 inhibitors that are known in the art, include: Chen et al. Mol Pharmacol. 2006, 70, 562; Sarver et al., J. Med. Chem. 2017, 62, 1793; Xie et al., J. Med. Chem. 2017, 60, 113734; and Igbe et al., Oncotarget, 2017, 8, 113734; and PCT applications: WO 2022235822, WO 20222084008, WO 2022135568, WO 2021176072, WO 2021171261, WO 2021149817, WO 2021148010, WO 2021147879, WO 2021143823, WO 2021143701, WO 2021143680, WO 2021121397, WO 2021119525, WO 2021115286, WO 2021110796, WO 2021088945, WO 2021073439, WO 2021061706, WO 2021061515, WO 2021043077, WO 2021033153, WO 2021028362, WO 2021033153, WO 2021028362, WO 2021018287, WO 2020259679, WO 2020249079, WO 2020210384, WO 2020201991, WO 2020181283, WO 2020177653, WO 2020165734, WO 2020165733, WO 2020165732, WO 2020156243, WO 2020156242, WO 2020108590, WO 2020104635, WO 2020094104, WO 2020094018, WO 2020081848, WO 2020073949, WO 2020073945, WO 2020072656, WO 2020065453, WO 2020065452, WO 2020063760, WO 2020061103, WO 2020061101, WO 2020033828, WO 2020033286, WO 2020022323, WO 2019233810, WO 2019213318, WO 2019183367, WO 2019183364, WO 2019182960, WO 2019167000, WO 2019165073, WO 2019158019, WO 2019152454, WO 2019051469, WO 2019051084, WO 2018218133, WO 2018172984, WO 2018160731, WO 2018136265, WO 2018136264, WO 2018130928, WO 2018129402, WO 2018081091, WO 2018057884, WO 2018013597, WO 2017216706, WO 2017211303, WO 2017210134, WO 2017156397, WO 2017100279, WO 2017079723, WO 2017078499, WO 2016203406, WO 2016203405, WO 2016203404, WO 2016196591, WO 2016191328, WO 2015107495, WO 2015107494, WO 2015107493, WO 2014176488, WO 2014113584, US 20210085677, U.S. Pat. Nos. 10,858,359, 10,934,302, 10,954,243, 10,988,466, 11,001,561, 11,033,547, U.S. Ser. No. 11/034,705, or U.S. Pat. No. 11,044,675, or a pharmaceutically acceptable salt, solvate, isomer (e.g., stereoisomer), prodrug, or tautomer thereof, each of which is incorporated herein by reference.

In some embodiments, a SHP2 inhibitor binds in the active site. In some embodiments, a SHP2 inhibitor is a mixed-type irreversible inhibitor. In some embodiments, a SHP2 inhibitor binds an allosteric site e.g., a non-covalent allosteric inhibitor. In some embodiments, a SHP2 inhibitor is a covalent SHP2 inhibitor, such as an inhibitor that targets the cysteine residue (C333) that lies outside the phosphatase's active site. In some embodiments a SHP2 inhibitor is a reversible inhibitor. In some embodiments, a SHP2 inhibitor is an irreversible inhibitor. In some embodiments, the SHP2 inhibitor is SHP099. In some embodiments, the SHP2 inhibitor is TNO155, having the structure:

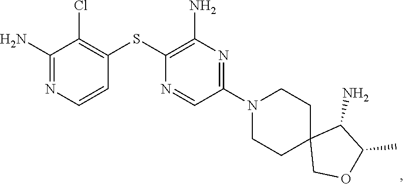 [SMILES: C[C@@H]1OCC2(CCN(c3cnc(Sc4ccnc(N)c4Cl)c(N)n3)CC2)[C@@H]1N]

or a pharmaceutically acceptable salt, solvate, isomer (e.g., stereoisomer), prodrug, or tautomer thereof. In some embodiments, the SHP2 inhibitor is RMC-4550. In some embodiments, the SHP2 inhibitor is RMC-4630, having the structure:

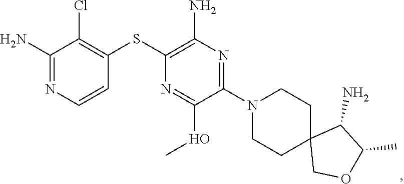 [SMILES: COc1nc(Sc2ccnc(N)c2Cl)c(N)nc1N1CCC2(CC1)CO[C@@H](C)[C@H]2N]

or a pharmaceutically acceptable salt, solvate, isomer (e.g., stereoisomer), prodrug, or tautomer thereof. In some embodiments, the SHP2 inhibitor is JAB-3068, having the structure

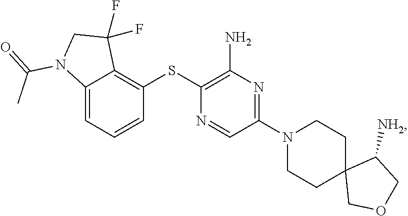 [SMILES: CC(=O)N1CC(F)(F)c2c(Sc3ncc(N4CCC5(CC4)COC[C@H]5N)nc3N)cccc21]

or a pharmaceutically acceptable salt, solvate, isomer (e.g., stereoisomer), prodrug, or tautomer thereof. In some embodiments, the SHP2 inhibitor is JAB-3312. In some embodiments, the SHP2 inhibitor is the following compound,

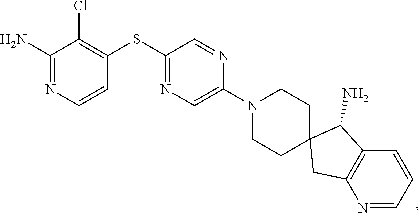 [SMILES: Nc1nccc(Sc2cnc(N3CCC4(CC3)Cc3ncccc3[C@H]4N)cn2)c1Cl]

or a pharmaceutically acceptable salt, solvate, isomer (e.g., stereoisomer), prodrug, or tautomer thereof. In some embodiments, the SHP2 inhibitor is RLY-1971, having the structure

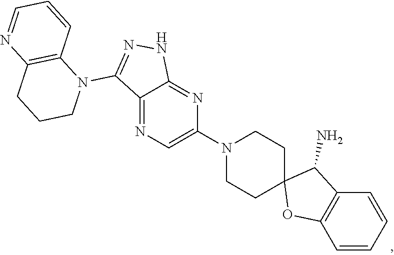 [SMILES: N[C@@H]1c2ccccc2OC12CCN(c1cnc3c(N4CCCc5ncccc54)n[nH]c3n1)CC2]

or a pharmaceutically acceptable salt, solvate, isomer (e.g., stereoisomer), prodrug, or tautomer thereof. In some embodiments, the SHP2 inhibitor is ERAS-601. In some embodiments, the SHP2 inhibitor is BBP-398.

In some embodiments, the additional therapeutic agent is selected from the group consisting of a MEK inhibitor, a HER2 inhibitor, a SHP2 inhibitor, a CDK4/6 inhibitor, an mTOR inhibitor, a SOS1 inhibitor, and a PD-L1 inhibitor. In some embodiments, the additional therapeutic agent is selected from the group consisting of a MEK inhibitor, a SHP2 inhibitor, and a PD-L1 inhibitor. See, e.g., Hallin et al., Cancer Discovery, DOI: 10.1158/2159-8290 (Oct. 28, 2019) and Canon et al., Nature, 575:217 (2019). In some embodiments, a Ras inhibitor of the present invention is used in combination with a MEK inhibitor and a SOS1 inhibitor. In some embodiments, a Ras inhibitor of the present invention is used in combination with a PD-L1 inhibitor and a SOS1 inhibitor. In some embodiments, a Ras inhibitor of the present invention is used in combination with a PD-L1 inhibitor and a SHP2 inhibitor. In some embodiments, a Ras inhibitor of the present invention is used in combination with a MEK inhibitor and a SHP2 inhibitor. In some embodiments, a Ras inhibitor of the present invention is used in combination with a SHP2 inhibitor and a Ras inhibitor that inhibits multiple Ras isoforms and/or mutants (e.g., RMC-6236). In some embodiments, the cancer is lung cancer, and the treatment comprises administration of a Ras inhibitor of the present invention in combination with a second or third therapeutic agent, such as a SHP2 inhibitor and a Ras inhibitor that inhibits multiple Ras isoforms and/or mutants. In some embodiments, the cancer is colorectal cancer, and the treatment comprises administration of a Ras inhibitor of the present invention in combination with a second or third therapeutic agent, such as a SHP2 inhibitor and a Ras inhibitor that inhibits multiple Ras isoforms and/or mutants. In some embodiments, a Ras inhibitor of the present invention is used in combination with an immunotherapy, optionally in combination with a chemotherapeutic agent.

Proteasome inhibitors are known in the art and include, but are not limited to, carfilzomib (Kyprolis®), bortezomib (Velcade®), and oprozomib.

Immune therapies include, but are not limited to, monoclonal antibodies, immunomodulatory imides (IMiDs), GITR agonists, genetically engineered T-cells (e.g., CAR-T cells), bispecific antibodies (e.g., BiTEs), and anti-PD-1, anti-PD-L1, anti-CTLA4, anti-LAGI, and anti-OX40 agents). Other immune therapies are known in the art.

Immunomodulatory agents (IMiDs) are a class of immunomodulatory drugs (drugs that adjust immune responses) containing an imide group. The IMiD class includes thalidomide and its analogues (lenalidomide, pomalidomide, and apremilast).

Exemplary anti-PD-1 antibodies and methods for their use are described by Goldberg et al., Blood 2007, 110(1):186-192; Thompson et al., Clin. Cancer Res. 2007, 13 (6):1757-1761; and WO06/121168 A1), as well as described elsewhere herein.

FGFR inhibitors are known in the art, such as pemigatinib and erdafitinib, including FGFR2 inhibitors and FGFR4 inhibitors. See, e.g., Cancers (Basel), 2021 June; 13 (12) 2968.

BET inhibitors are known in the art, such as romidepsin, panobinostat and belinostat. See, e.g., British J. Cancer 124:1478 (2021).

PRMT5i inhibitors are known in the art, such as PF-0693999, PJ-68 and MRTX1719. See, e.g., Biomed. Pharmacotherapy 144:112252 (2021).

MAT2A inhibitors are known in the art, such as AG-270 and IDE397. See, e.g., Exp Opin Ther Patents (2022) DOI: 10.1080/13543776.2022.2119127.

GITR agonists include, but are not limited to, GITR fusion proteins and anti-GITR antibodies (e.g., bivalent anti-GITR antibodies), such as, a GITR fusion protein described in U.S. Pat. Nos. 6,111,090, 8,586,023, WO2010/003118 and WO2011/090754; or an anti-GITR antibody described, e.g., in U.S. Pat. No. 7,025,962, EP 1947183, U.S. Pat. Nos. 7,812,135, 8,388,967, 8,591,886, 7,618,632, EP 1866339, and WO2011/028683, WO2013/039954, WO05/007190, WO07/133822, WO05/055808, WO99/40196, WO01/03720, WO99/20758, WO06/083289, WO05/115451, and WO2011/051726.

Another example of a therapeutic agent that may be used in combination with the compounds of the invention is an anti-angiogenic agent. Anti-angiogenic agents are known in the art and are inclusive of, but not limited to, in vitro synthetically prepared chemical compositions, antibodies, antigen binding regions, radionuclides, and combinations and conjugates thereof. An anti-angiogenic agent can be an agonist, antagonist, allosteric modulator, toxin or, more generally, may act to inhibit or stimulate its target (e.g., receptor or enzyme activation or inhibition), and thereby promote cell death or arrest cell growth. In some embodiments, the one or more additional therapies include an anti-angiogenic agent.

Anti-angiogenic agents can be MMP-2 (matrix-metalloproteinase 2) inhibitors, MMP-9 (matrix-metalloproteinase 9) inhibitors, and COX-II (cyclooxygenase 11) inhibitors. Non-limiting examples of anti-angiogenic agents include rapamycin, temsirolimus (CCI-779), everolimus (RAD001), sorafenib, sunitinib, and bevacizumab. Examples of useful COX-II inhibitors include alecoxib, valdecoxib, and rofecoxib. Examples of useful matrix metalloproteinase inhibitors are described in WO96/33172, WO96/27583, WO98/07697, WO98/03516, WO98/34918, WO98/34915, WO98/33768, WO98/30566, WO90/05719, WO99/52910, WO99/52889, WO99/29667, WO99007675, EP0606046, EP0780386, EP1786785, EP1181017, EP0818442, EP1004578, and US20090012085, and U.S. Pat. Nos. 5,863,949 and 5,861,510. Preferred MMP-2 and MMP-9 inhibitors are those that have little or no activity inhibiting MMP-1. More preferred, are those that selectively inhibit MMP-2 or AMP-9 relative to the other matrix-metalloproteinases (i.e., MAP-1, MMP-3, MMP-4, MMP-5, MMP-6, MMP-7, MMP-8, MMP-10, MMP-11, MMP-12, and MMP-13). Some specific examples of MMP inhibitors are AG-3340, RO 32-3555, and RS 13-0830.

Further exemplary anti-angiogenic agents include KDR (kinase domain receptor) inhibitory agents (e.g., antibodies and antigen binding regions that specifically bind to the kinase domain receptor), anti-VEGF agents (e.g., antibodies or antigen binding regions that specifically bind VEGF (e.g., bevacizumab), or soluble VEGF receptors or a ligand binding region thereof) such as VEGF-TRAP™, and anti-VEGF receptor agents (e.g., antibodies or antigen binding regions that specifically bind thereto), VEGF inhibitors, EGFR inhibitory agents (e.g., antibodies or antigen binding regions that specifically bind thereto) such as Vectibix® (panitumumab), erlotinib (Tarceva®), anti-Angl and anti-Ang2 agents (e.g., antibodies or antigen binding regions specifically binding thereto or to their receptors, e.g., Tie2/Tek), and anti-Tie2 kinase inhibitory agents (e.g., antibodies or antigen binding regions that specifically bind thereto). Other anti-angiogenic agents include Campath, IL-8, B-FGF, Tek antagonists (US2003/0162712; U.S. Pat. No. 6,413,932), anti-TWEAK agents (e.g., specifically binding antibodies or antigen binding regions, or soluble TWEAK receptor antagonists; see U.S. Pat. No. 6,727,225), ADAM distintegrin domain to antagonize the binding of integrin to its ligands (US 2002/0042368), specifically binding anti-eph receptor or anti-ephrin antibodies or antigen binding regions (U.S. Pat. Nos. 5,981,245; 5,728,813; 5,969,110; 6,596,852; 6,232,447; 6,057,124 and patent family members thereof), and anti-PDGF-BB antagonists (e.g., specifically binding antibodies or antigen binding regions) as well as antibodies or antigen binding regions specifically binding to PDGF-BB ligands, and PDGFR kinase inhibitory agents (e.g., antibodies or antigen binding regions that specifically bind thereto). Additional anti-angiogenic agents include: SD-7784 (Pfizer, USA); cilengitide (Merck KGaA, Germany, EPO 0770622); pegaptanib octasodium, (Gilead Sciences, USA); Alphastatin, (BioActa, UK); M-PGA, (Celgene, USA, U.S. Pat. No. 5,712,291); ilomastat, (Arriva, USA, U.S. Pat. No. 5,892,112); emaxanib, (Pfizer, USA, U.S. Pat. No. 5,792,783); vatalanib, (Novartis, Switzerland); 2-methoxyestradiol (EntreMed, USA); TLC ELL-12 (Elan, Ireland); anecortave acetate (Alcon, USA); alpha-D148 Mab (Amgen, USA); CEP-7055 (Cephalon, USA); anti-Vn Mab (Crucell, Netherlands), DACantiangiogenic (ConjuChem, Canada); Angiocidin (InKine Pharmaceutical, USA); KM-2550 (Kyowa Hakko, Japan); SU-0879 (Pfizer, USA); CGP-79787 (Novartis, Switzerland, EP 0970070); ARGENT technology (Ariad, USA); YIGSR-Stealth (Johnson & Johnson, USA); fibrinogen-E fragment (BioActa, UK); angiogenic inhibitor (Trigen, UK); TBC-1635 (Encysive Pharmaceuticals, USA); SC-236 (Pfizer, USA); ABT-567 (Abbott, USA); Metastatin (EntreMed, USA); maspin (Sosei, Japan); 2-methoxyestradiol (Oncology Sciences Corporation, USA); ER-68203-00 (IV AX, USA); BeneFin (Lane Labs, USA); Tz-93 (Tsumura, Japan); TAN-1120 (Takeda, Japan); FR-111142 (Fujisawa, Japan, JP 02233610); platelet factor 4 (RepliGen, USA, EP 407122); vascular endothelial growth factor antagonist (Borean, Denmark); bevacizumab (pINN) (Genentech, USA); angiogenic inhibitors (SUGEN, USA); XL 784 (Exelixis, USA); XL 647 (Exelixis, USA); MAb, alpha5beta3 integrin, second generation (Applied Molecular Evolution, USA and MedImmune, USA); enzastaurin hydrochloride (Lilly, USA); CEP 7055 (Cephalon, USA and Sanofi-Synthelabo, France); BC 1 (Genoa Institute of Cancer Research, Italy); rBPI 21 and BPI-derived antiangiogenic (XOMA, USA); PI 88 (Progen, Australia); cilengitide (Merck KGaA, German; Munich Technical University, Germany, Scripps Clinic and Research Foundation, USA); AVE 8062 (Ajinomoto, Japan); AS 1404 (Cancer Research Laboratory, New Zealand); SG 292, (Telios, USA); Endostatin (Boston Childrens Hospital, USA); ATN 161 (Attenuon, USA); 2-methoxyestradiol (Boston Childrens Hospital, USA); ZD 6474, (AstraZeneca, UK); ZD 6126, (Angiogene Pharmaceuticals, UK); PPI 2458, (Praecis, USA); AZD 9935, (AstraZeneca, UK); AZD 2171, (AstraZeneca, UK); vatalanib (pINN), (Novartis, Switzerland and Schering AG, Germany); tissue factor pathway inhibitors, (EntreMed, USA); pegaptanib (Pinn), (Gilead Sciences, USA); xanthorrhizol, (Yonsei University, South Korea); vaccine, gene-based, VEGF-2, (Scripps Clinic and Research Foundation, USA); SPV5.2, (Supratek, Canada); SDX 103, (University of California at San Diego, USA); PX 478, (ProIX, USA); METASTATIN, (EntreMed, USA); troponin I, (Harvard University, USA); SU 6668, (SUGEN, USA); OXI 4503, (OXIGENE, USA); o-guanidines, (Dimensional Pharmaceuticals, USA); motuporamine C, (British Columbia University, Canada); CDP 791, (Celltech Group, UK); atiprimod (pINN), (GlaxoSmithKline, UK); E 7820, (Eisai, Japan); CYC 381, (Harvard University, USA); AE 941, (Aeterna, Canada); vaccine, angiogenic, (EntreMed, USA); urokinase plasminogen activator inhibitor, (Dendreon, USA); oglufanide (pINN), (Melmotte, USA); HIF-lalfa inhibitors, (Xenova, UK); CEP 5214, (Cephalon, USA); BAY RES 2622, (Bayer, Germany); Angiocidin, (InKine, USA); A6, (Angstrom, USA); KR 31372, (Korea Research Institute of Chemical Technology, South Korea); GW 2286, (GlaxoSmithKline, UK); EHT 0101, (ExonHit, France); CP 868596, (Pfizer, USA); CP 564959, (OSI, USA); CP 547632, (Pfizer, USA); 786034, (GlaxoSmithKline, UK); KRN 633, (Kirin Brewery, Japan); drug delivery system, intraocular, 2-methoxyestradiol; anginex (Maastricht University, Netherlands, and Minnesota University, USA); ABT 510 (Abbott, USA); AAL 993 (Novartis, Switzerland); VEGI (ProteomTech, USA); tumor necrosis factor-alpha inhibitors; SU 11248 (Pfizer, USA and SUGEN USA); ABT 518, (Abbott, USA); YH16 (Yantai Rongchang, China); S-3APG (Boston Childrens Hospital, USA and EntreMed, USA); MAb, KDR (ImClone Systems, USA); MAb, alpha5 beta (Protein Design, USA); KDR kinase inhibitor (Celltech Group, UK, and Johnson & Johnson, USA); GFB 116 (South Florida University, USA and Yale University, USA); CS 706 (Sankyo, Japan); combretastatin A4 prodrug (Arizona State University, USA); chondroitinase AC (IBEX, Canada); BAY RES 2690 (Bayer, Germany); AGM 1470 (Harvard University, USA, Takeda, Japan, and TAP, USA); AG 13925 (Agouron, USA); Tetrathiomolybdate (University of Michigan, USA); GCS 100 (Wayne State University, USA) CV 247 (Ivy Medical, UK); CKD 732 (Chong Kun Dang, South Korea); irsogladine, (Nippon Shinyaku, Japan); RG 13577 (Aventis, France); WX 360 (Wilex, Germany); squalamine, (Genaera, USA); RPI 4610 (Sirna, USA); heparanase inhibitors (InSight, Israel); KL 3106 (Kolon, South Korea); Honokiol (Emory University, USA); ZK CDK (Schering AG, Germany); ZK Angio (Schering AG, Germany); ZK 229561 (Novartis, Switzerland, and Schering AG, Germany); XMP 300 (XOMA, USA); VGA 1102 (Taisho, Japan); VE-cadherin-2 antagonists (ImClone Systems, USA); Vasostatin (National Institutes of Health, USA); Flk-1 (ImClone Systems, USA); TZ 93 (Tsumura, Japan); TumStatin (Beth Israel Hospital, USA); truncated soluble FLT 1 (vascular endothelial growth factor receptor 1) (Merck & Co, USA); Tie-2 ligands (Regeneron, USA); and thrombospondin 1 inhibitor (Allegheny Health, Education and Research Foundation, USA).

Further examples of therapeutic agents that may be used in combination with compounds of the invention include agents (e.g., antibodies, antigen binding regions, or soluble receptors) that specifically bind and inhibit the activity of growth factors, such as antagonists of hepatocyte growth factor (HGF, also known as Scatter Factor), and antibodies or antigen binding regions that specifically bind its receptor, c-Met. Such agents are known in the art.

Another example of a therapeutic agent that may be used in combination with compounds of the invention is an autophagy inhibitor. Autophagy inhibitors are known in the art and include, but are not limited to chloroquine, 3-methyladenine, hydroxychloroquine (Plaquenil™), bafilomycin A1, 5-amino-4-imidazole carboxamide riboside (AICAR), okadaic acid, autophagy-suppressive algal toxins which inhibit protein phosphatases of type 2A or type 1, analogues of CAMP, and drugs which elevate cAMP levels such as adenosine, LY204002, N6-mercaptopurine riboside, and vinblastine. In addition, antisense or siRNA that inhibits expression of proteins including but not limited to ATG5 (which are implicated in autophagy), may also be used. In some embodiments, the one or more additional therapies include an autophagy inhibitor.

Another example of a therapeutic agent that may be used in combination with compounds of the invention is an anti-neoplastic agent, which are known in the art. In some embodiments, the one or more additional therapies include an anti-neoplastic agent. Non-limiting examples of anti-neoplastic agents include acemannan, aclarubicin, aldesleukin, alemtuzumab, alitretinoin, altretamine, amifostine, aminolevulinic acid, amrubicin, amsacrine, anagrelide, anastrozole, ancer, ancestim, arglabin, arsenic trioxide, BAM-002 (Novelos), bexarotene, bicalutamide, broxuridine, capecitabine, celmoleukin, cetrorelix, cladribine, clotrimazole, cytarabine ocfosfate, DA 3030 (Dong-A), daclizumab, denileukin diftitox, deslorelin, dexrazoxane, dilazep, docetaxel, docosanol, doxercalciferol, doxifluridine, doxorubicin, bromocriptine, carmustine, cytarabine, fluorouracil, HIT diclofenac, interferon alfa, daunorubicin, doxorubicin, tretinoin, edelfosine, edrecolomab, eflornithine, emitefur, epirubicin, epoetin beta, etoposide phosphate, exemestane, exisulind, fadrozole, filgrastim, finasteride, fludarabine phosphate, formestane, fotemustine, gallium nitrate, gemcitabine, gemtuzumab zogamicin, gimeracil/oteracil/tegafur combination, glycopine, goserelin, heptaplatin, human chorionic gonadotropin, human fetal alpha fetoprotein, ibandronic acid, idarubicin, (imiquimod, interferon alfa, interferon alfa, natural, interferon alfa-2, interferon alfa-2a, interferon alfa-2b, interferon alfa-NI, interferon alfa-n3, interferon alfacon-1, interferon alpha, natural, interferon beta, interferon beta-la, interferon beta-lb, interferon gamma, natural interferon gamma-la, interferon gamma-lb, interleukin-1 beta, iobenguane, irinotecan, irsogladine, lanreotide, LC 9018 (Yakult), leflunomide, lenograstim, lentinan sulfate, letrozole, leukocyte alpha interferon, leuprorelin, levamisole+fluorouracil, liarozole, lobaplatin, lonidamine, lovastatin, masoprocol, melarsoprol, metoclopramide, mifepristone, miltefosine, mirimostim, mismatched double stranded RNA, mitoguazone, mitolactol, mitoxantrone, molgramostim, nafarelin, naloxone+pentazocine, nartograstim, nedaplatin, nilutamide, noscapine, novel erythropoiesis stimulating protein, NSC 631570 octreotide, oprelvekin, osaterone, oxaliplatin, paclitaxel, pamidronic acid, pegaspargase, peginterferon alfa-2b, pentosan polysulfate sodium, pentostatin, picibanil, pirarubicin, rabbit antithymocyte polyclonal antibody, polyethylene glycol interferon alfa-2a, porfimer sodium, raloxifene, raltitrexed, rasburiembodiment, rhenium Re 186 etidronate, RII retinamide, rituximab, romurtide, samarium (153 Sm) lexidronam, sargramostim, sizofiran, sobuzoxane, sonermin, strontium-89 chloride, suramin, tasonermin, tazarotene, tegafur, temoporfin, temozolomide, teniposide, tetrachlorodecaoxide, thalidomide, thymalfasin, thyrotropin alfa, topotecan, toremifene, tositumomab-iodine 131, trastuzumab, treosulfan, tretinoin, trilostane, trimetrexate, triptorelin, tumor necrosis factor alpha, natural, ubenimex, bladder cancer vaccine, Maruyama vaccine, melanoma lysate vaccine, valrubicin, verteporfin, vinorelbine, virulizin, zinostatin stimalamer, or zoledronic acid; abarelix; AE 941 (Aeterna), ambamustine, antisense oligonucleotide, bcl-2 (Genta), APC 8015 (Dendreon), decitabine, dexaminoglutethimide, diaziquone, EL 532 (Elan), EM 800 (Endorecherche), eniluracil, etanidazole, fenretinide, filgrastim SD01 (Amgen), fulvestrant, galocitabine, gastrin 17 immunogen, HLA-B7 gene therapy (Vical), granulocyte macrophage colony stimulating factor, histamine dihydrochloride, ibritumomab tiuxetan, ilomastat, IM 862 (Cytran), interleukin-2, iproxifene, LDI 200 (Milkhaus), leridistim, lintuzumab, CA 125 MAb (Biomira), cancer MAb (Japan Pharmaceutical Development), HER-2 and Fc MAb (Medarex), idiotypic 105AD7 MAb (CRC Technology), idiotypic CEA MAb (Trilex), LYM-1-iodine 131 MAb (Techni clone), polymorphic epithelial mucin-yttrium 90 MAb (Antisoma), marimastat, menogaril, mitumomab, motexafin gadolinium, MX 6 (Galderma), nelarabine, nolatrexed, P 30 protein, pegvisomant, pemetrexed, porfiromycin, prinomastat, RL 0903 (Shire), rubitecan, satraplatin, sodium phenylacetate, sparfosic acid, SRL 172 (SR Pharma), SU 5416 (SUGEN), TA 077 (Tanabe), tetrathiomolybdate, thaliblastine, thrombopoietin, tin ethyl etiopurpurin, tirapazamine, cancer vaccine (Biomira), melanoma vaccine (New York University), melanoma vaccine (Sloan Kettering Institute), melanoma oncolysate vaccine (New York Medical College), viral melanoma cell lysates vaccine (Royal Newcastle Hospital), or valspodar.

Additional examples of therapeutic agents that may be used in combination with compounds of the invention include ipilimumab (Yervoy®); tremelimumab; galiximab; nivolumab, also known as BMS-936558 (Opdivo®); pembrolizumab (Keytruda®); avelumab (Bavencio®); AMP224; BMS-936559; MPDL3280A, also known as RG7446; MEDI-570; AMG557; MGA271; IMP321; BMS-663513; PF-05082566; CDX-1127; anti-OX40 (Providence Health Services); huMADOX40L; atacicept; CP-870893; lucatumumab; dacetuzumab; muromonab-CD3; ipilumumab; MEDI4736 (Imfinzi®); MSB0010718C; AMP 224; adalimumab (Humira®); ado-trastuzumab emtansine (Kadcyla®); aflibercept (Eylea®); alemtuzumab (Campath®); basiliximab (Simulect®); belimumab (Benlysta®); basiliximab (Simulect®); belimumab (Benlysta®); brentuximab vedotin (Adcetris®); canakinumab (Ilaris®); certolizumab pegol (Cimzia®); daclizumab (Zenapax®); daratumumab (Darzalex®); denosumab (Prolia®); eculizumab (Soliris®); efalizumab (Raptiva®); gemtuzumab ozogamicin (Mylotarg®); golimumab (Simponi®); ibritumomab tiuxetan (Zevalin®); infliximab (Remicade®); motavizumab (Numax®); natalizumab (Tysabri®); obinutuzumab (Gazyva®); ofatumumab (Arzerra®); omalizumab (Xolair®); palivizumab (Synagis®); pertuzumab (Perjeta®); pertuzumab (Perjeta®); ranibizumab (Lucentis®); raxibacumab (Abthrax®); tocilizumab (Actemra®); tositumomab; tositumomab-i-131; tositumomab and tositumomab-i-131 (Bexxar®); ustekinumab (Stelara®); AMG 102; AMG 386; AMG 479; AMG 655; AMG 706; AMG 745; and AMG 951.

The compounds described herein can be used in combination with the agents disclosed herein or other suitable agents, depending on the condition being treated. Hence, in some embodiments the one or more compounds of the disclosure will be co-administered with other therapies as described herein. When used in combination therapy, the compounds described herein may be administered with the second agent simultaneously or separately. This administration in combination can include simultaneous administration of the two agents in the same dosage form, simultaneous administration in separate dosage forms, and separate administration. That is, a compound described herein and any of the agents described herein can be formulated together in the same dosage form and administered simultaneously. Alternatively, a compound of the invention and any of the therapies described herein can be simultaneously administered, wherein both the agents are present in separate formulations. In another alternative, a compound of the present disclosure can be administered and followed by any of the therapies described herein, or vice versa. In some embodiments of the separate administration protocol, a compound of the invention and any of the therapies described herein are administered a few minutes apart, or a few hours apart, or a few days apart.

In some embodiments of any of the methods described herein, the first therapy (e.g., a compound of the invention) and one or more additional therapies are administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours, up to 24 hours, or up to 1-7, 1-14, 1-21 or 1-30 days before or after the one or more additional therapies.

The invention also features kits including (a) a pharmaceutical composition including an agent (e.g., a compound of the invention) described herein, and (b) a package insert with instructions to perform any of the methods described herein. In some embodiments, the kit includes (a) a pharmaceutical composition including an agent (e.g., a compound of the invention) described herein, (b) one or more additional therapies (e.g., non-drug treatment or therapeutic agent), and (c) a package insert with instructions to perform any of the methods described herein.

As one aspect of the present invention contemplates the treatment of the disease or symptoms associated therewith with a combination of pharmaceutically active compounds that may be administered separately, the invention further relates to combining separate pharmaceutical compositions in kit form. The kit may comprise two separate pharmaceutical compositions: a compound of the present invention, and one or more additional therapies. The kit may comprise a container for containing the separate compositions such as a divided bottle or a divided foil packet. Additional examples of containers include syringes, boxes, and bags. In some embodiments, the kit may comprise directions for the use of the separate components. The kit form is particularly advantageous when the separate components are preferably administered in different dosage forms (e.g., oral and parenteral), are administered at different dosage intervals, or when titration of the individual components of the combination is desired by the prescribing health care professional.

### NUMBERED EMBODIMENTS

1. A compound, or pharmaceutically acceptable salt thereof, having the structure of Formula I:

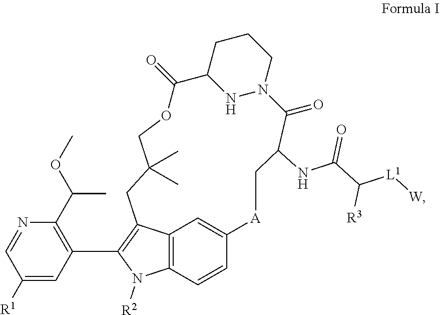

wherein A is optionally substituted 3 to 6-membered heterocycloalkylene, optionally substituted 3 to 6-membered cycloalkylene, optionally substituted 6-membered arylene, or optionally substituted 5 to 10-membered heteroarylene;
L1 is absent or a linker;
W is a cross-linking group comprising a vinyl ketone, vinyl sulfone, ynone, or an alkynyl sulfone;
R¹ is hydrogen, optionally substituted 3 to 10-membered heterocycloalkyl, or optionally substituted C₁-C₆ heteroalkyl;
R² is optionally substituted C₁-C₆ alkyl; and
R³ is optionally substituted C₁-C₆ alkyl or optionally substituted C₁-C₃ heteroalkyl.
2. The compound of embodiment 1, or pharmaceutically acceptable salt thereof, wherein A is optionally substituted thiazole-diyl, optionally substituted oxazole-diyl, optionally substituted morpholine-diyl, optionally substituted pyrrolidine-diyl, optionally substituted pyridine-diyl, optionally substituted azetidine-diyl, optionally substituted pyrazine-diyl, optionally substituted pyrimidine-diyl, optionally substituted piperidine-diyl, optionally substituted oxadiazole-diyl, optionally substituted thiadiazole-diyl, optionally substituted triazole-diyl, optionally substituted thiomorpholine-diyl, or optionally substituted phenylene.
3. The compound of embodiment 1 or 2, or pharmaceutically acceptable salt thereof, having the structure of Formula II-1:

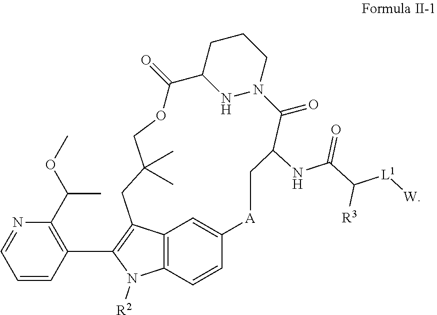

4. The compound of embodiment 1 or 2, or pharmaceutically acceptable salt thereof, having the structure of Formula II-2:

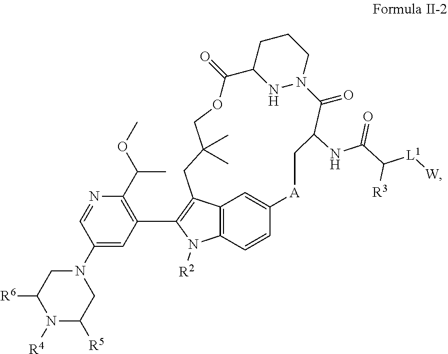

wherein R⁴, R⁵, and R⁶ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered heterocycloalkyl; or
R⁴ and R⁵ combine with the atoms to which they are attached to form an optionally substituted 3 to 8-membered cycloalkyl or an optionally substituted 3 to 8-membered heterocycloalkyl; or
R⁴ and R⁶ combine with the atoms to which they are attached to form an optionally substituted 3 to 8-membered cycloalkyl or an optionally substituted 3 to 8-membered heterocycloalkyl.
5. The compound of embodiment 4, or pharmaceutically acceptable salt thereof, having the structure of Formula II-3:

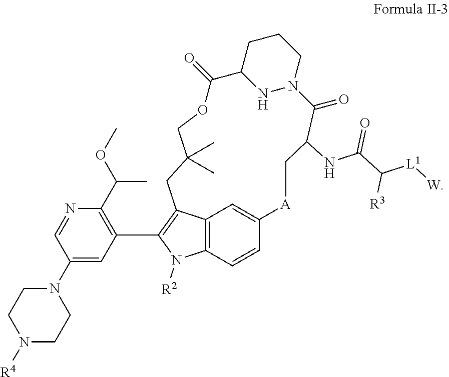

6. The compound of embodiment 4, or pharmaceutically acceptable salt thereof, having the structure of Formula II-4:

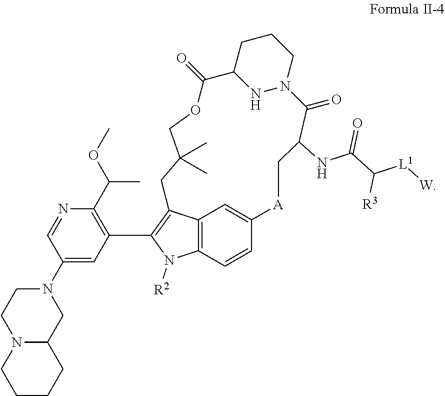

7. The compound of embodiment 4, or pharmaceutically acceptable salt thereof, having the structure of Formula II-4b:

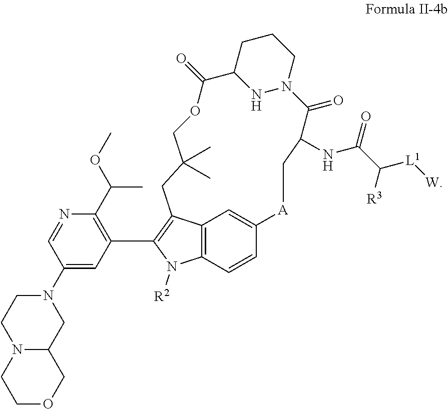

8. The compound of any one of embodiments 1 to 7, or pharmaceutically acceptable salt thereof, wherein R² is:

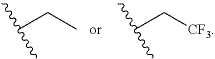

9. The compound of any one of embodiments 1 to 8, or pharmaceutically acceptable salt thereof, wherein R³ is optionally substituted C₁-C₆ alkyl.
10. The compound of embodiment 9, or pharmaceutically acceptable salt thereof, wherein R³ is:

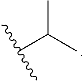 [SMILES: *C(C)C]

11. The compound of any one of embodiments 1 to 8, or pharmaceutically acceptable salt thereof, wherein R³ is optionally substituted C₁-C₃ heteroalkyl.
12. The compound of embodiment 11, or pharmaceutically acceptable salt thereof, wherein R³ is:

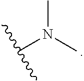 [SMILES: *N(C)C]

13. The compound of any one of embodiments 1 to 12, or pharmaceutically acceptable salt thereof, wherein A is optionally substituted 5 to 10-membered heteroarylene.
14. The compound of embodiment 13, or pharmaceutically acceptable salt thereof, wherein A is:

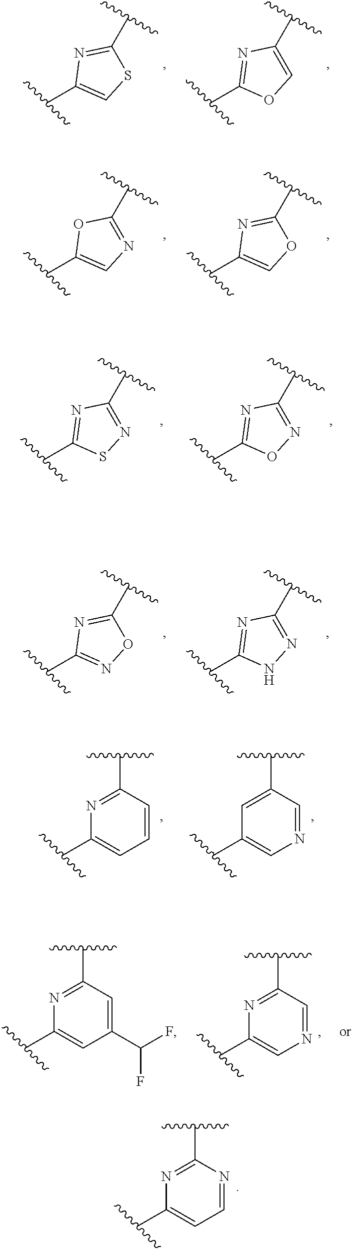

15. The compound of any one of embodiments 1 to 12, or pharmaceutically acceptable salt thereof, wherein A is optionally substituted phenylene.
16. The compound of embodiment 15, or pharmaceutically acceptable salt thereof, wherein A is:

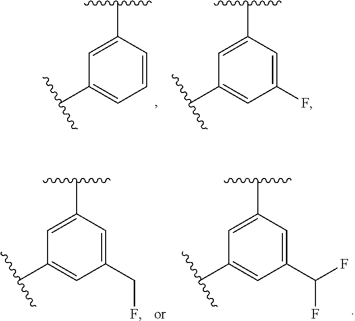

17. The compound of any one of embodiments 1 to 12, or pharmaceutically acceptable salt thereof, wherein A is optionally substituted 3 to 6-membered heterocycloalkylene.
18. The compound of embodiment 17, or pharmaceutically acceptable salt thereof, wherein A is:

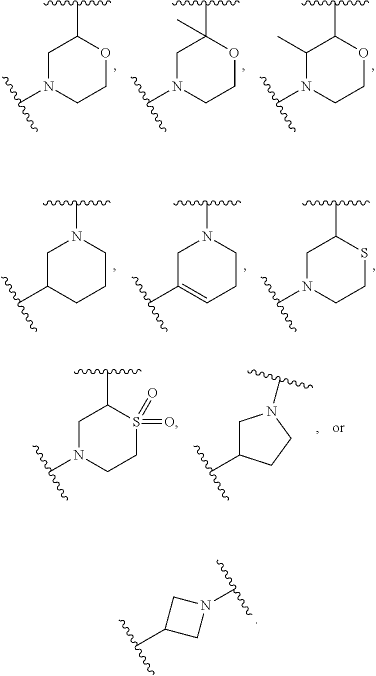

19. The compound of embodiment 17 or pharmaceutically acceptable salt thereof, wherein A is:

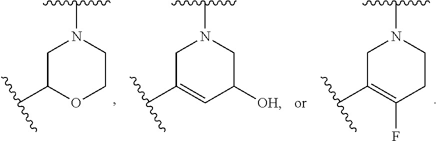

20. The compound of any one of embodiments 1 to 19, or pharmaceutically acceptable salt thereof, wherein the linker is the structure of Formula III:

A¹-(B¹)_{f}-(C¹)_{g}-(B²)ₕ-(D¹)-(B³)ᵢ-(C²)ⱼ-(B⁴)ₖ-A² Formula III,
wherein A¹ is a bond between the linker and CH(R³); A² is a bond between W and the linker; B¹, B², B³, and B⁴ each, independently, is selected from optionally substituted C₁-C₂ alkylene, optionally substituted C₁-C₃ heteroalkylene, O, S, and NR^{N}; each R^{N} is, independently, hydrogen, optionally substituted C₁-C₄ alkyl, optionally substituted C₂-C₄ alkenyl, optionally substituted C₂-C₄ alkynyl, optionally substituted 3 to 14-membered heterocycloalkyl, optionally substituted 6 to 10-membered aryl, or optionally substituted C₁-C₇ heteroalkyl; C¹ and C² are each, independently, selected from carbonyl, thiocarbonyl, sulphonyl, or phosphoryl; f, g, h, i, j, and k are each, independently, 0 or 1; and D1 is optionally substituted C₁-C₁₀ alkylene, optionally substituted C₂-C₁₀ alkenylene, optionally substituted C₂-C₁₀ alkynylene, optionally substituted 3 to 14-membered heterocycloalkylene, optionally substituted 5 to 10-membered heteroarylene, optionally substituted 3 to 8-membered cycloalkylene, optionally substituted 6 to 10-membered arylene, optionally substituted C₂-C₁₀ polyethylene glycolene, or optionally substituted C₁-C₁₀ heteroalkylene, or a chemical bond linking A¹-(B¹)_{f}-(C¹)_{g}-(B²)ₕ- to -(B³)ᵢ-(C²)ⱼ-(B⁴)ₖ-A².
21. The compound of any one of embodiments 1 to 20, or pharmaceutically acceptable salt thereof, wherein the linker is or comprises a cyclic moiety.
22. The compound of embodiment 21, or pharmaceutically acceptable salt thereof, wherein the linker has the structure of Formula IIIa:

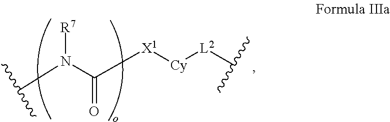

wherein o is 0 or 1;
R⁷ is hydrogen, optionally substituted C₁-C₆ alkyl, optionally substituted 3 to 8-membered cycloalkylene, or optionally substituted 3 to 8-membered heterocycloalkylene;
X¹ is absent, optionally substituted C₁-C₄ alkylene, O, NCH₃, or optionally substituted C₁-C₄ heteroalkylene;
Cy is optionally substituted 3 to 8-membered cycloalkylene, optionally substituted 3 to 12-membered heterocycloalkylene, optionally substituted 6-10 membered arylene, or optionally substituted 5 to 10-membered heteroarylene; and
L² is absent, —SO₂—, —NH—, optionally substituted C₁-C₄ alkylene, optionally substituted C₁-C₄ heteroalkylene, or optionally substituted 3 to 6-membered heterocycloalkylene.
23. The compound of embodiment 22, or pharmaceutically acceptable salt thereof, wherein the linker is selected from, or a stereoisomer thereof:

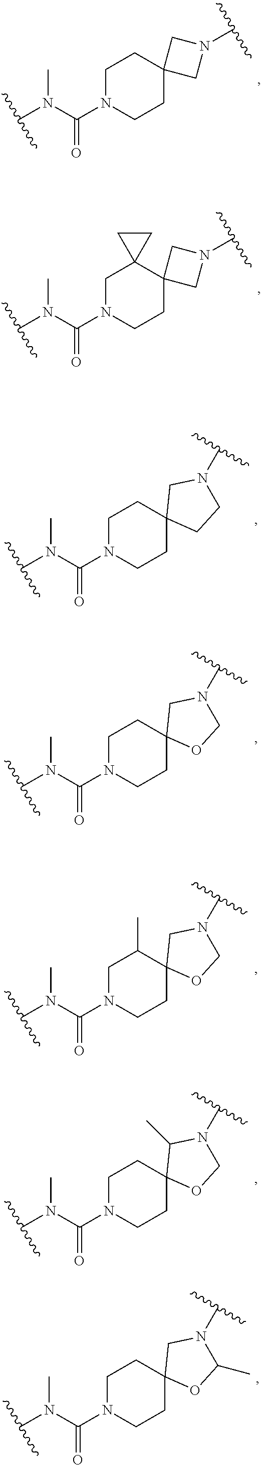

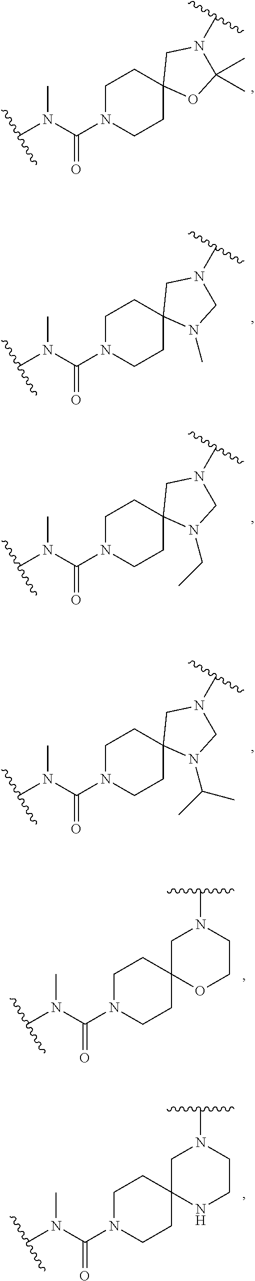 [SMILES: *N(C)C(=O)N1CCC2(CC1)CN(*)C(C)(C)O2 ; *N1CN(C)C2(CCN(C(=O)N(*)C)CC2)C1 ; *N1CN(CC)C2(CCN(C(=O)N(*)C)CC2)C1 ; *N1CN(C(C)C)C2(CCN(C(=O)N(*)C)CC2)C1 ; *N1CCOC2(CCN(C(=O)N(*)C)CC2)C1 ; *N1CCNC2(CCN(C(=O)N(*)C)CC2)C1]

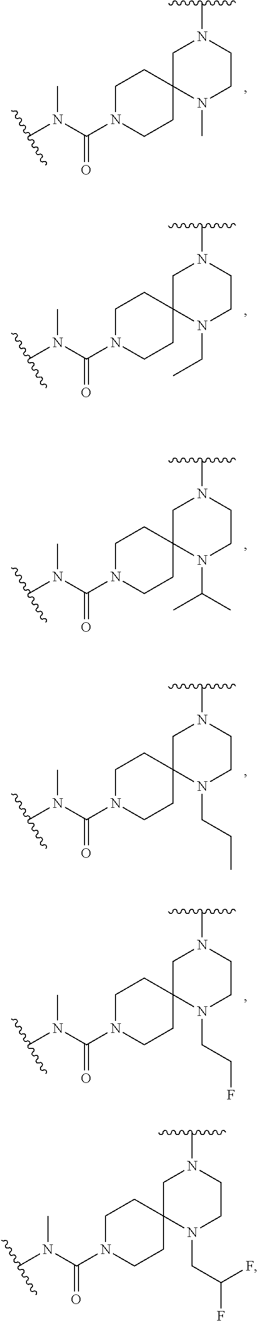 [SMILES: *N1CCN(C)C2(CCN(C(=O)N(*)C)CC2)C1 ; *N1CCN(CC)C2(CCN(C(=O)N(*)C)CC2)C1 ; *N1CCN(C(C)C)C2(CCN(C(=O)N(*)C)CC2)C1 ; *N1CCN(CCC)C2(CCN(C(=O)N(*)C)CC2)C1 ; *N1CCN(CCF)C2(CCN(C(=O)N(*)C)CC2)C1 ; *N1CCN(CC(F)F)C2(CCN(C(=O)N(*)C)CC2)C1]

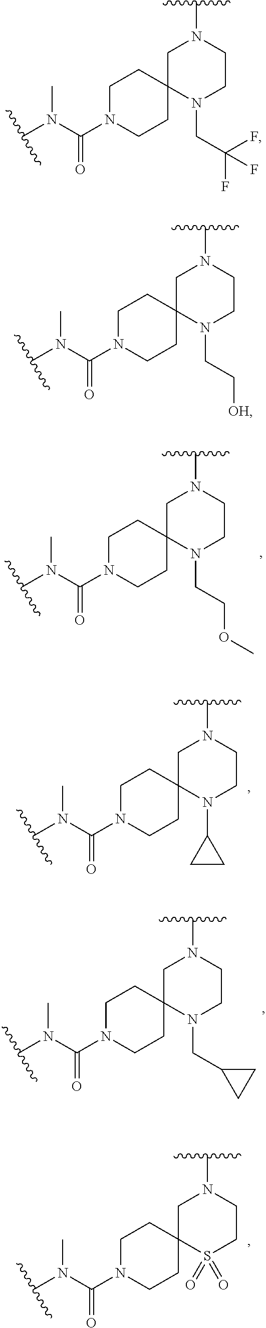

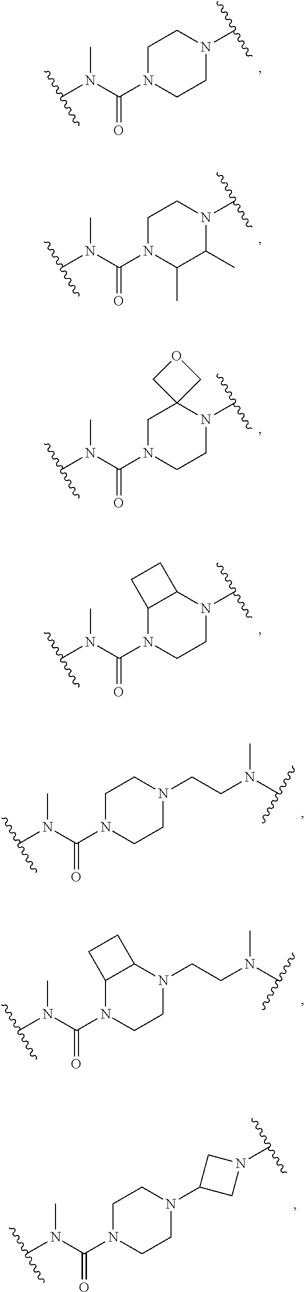

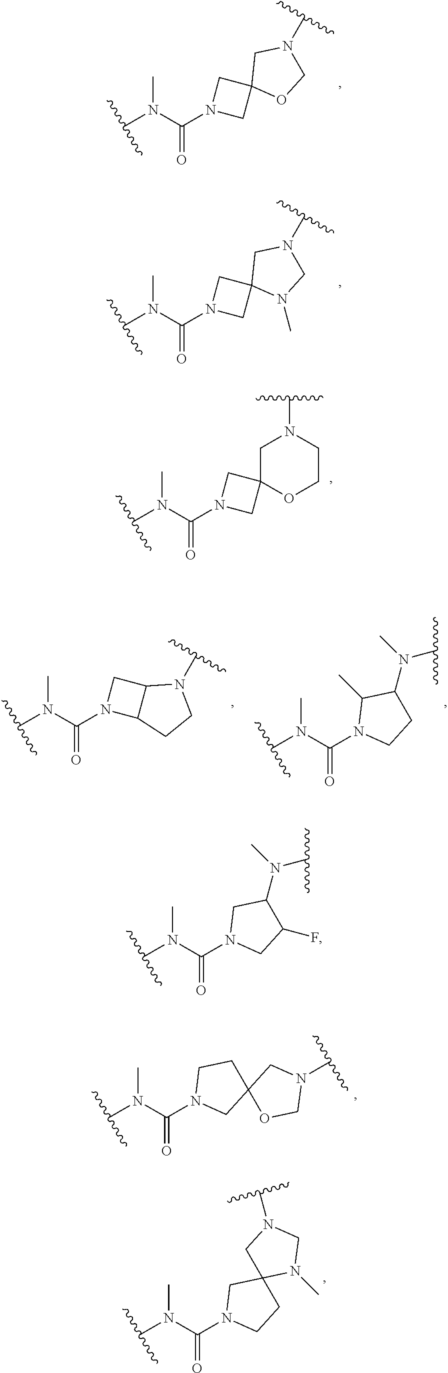

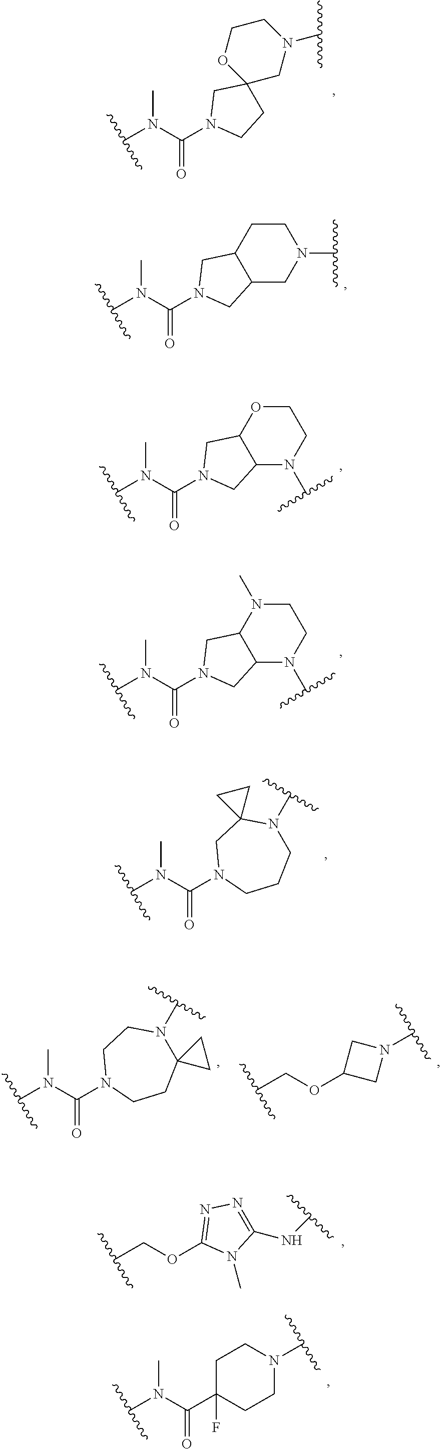

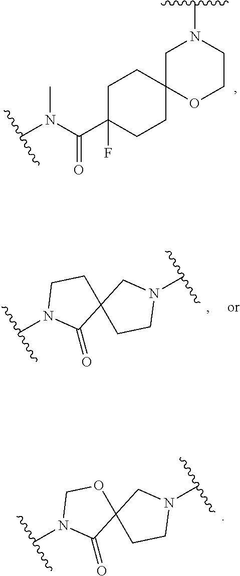 [SMILES: *N1CCOC2(CCC(F)(C(=O)N(*)C)CC2)C1 ; *N1CCC2(CCN(*)C2=O)C1 ; *N1CCC2(C1)OCN(*)C2=O]

24. The compound of embodiment 22, or pharmaceutically acceptable salt thereof, wherein the linker is selected from, or a stereoisomer thereof:

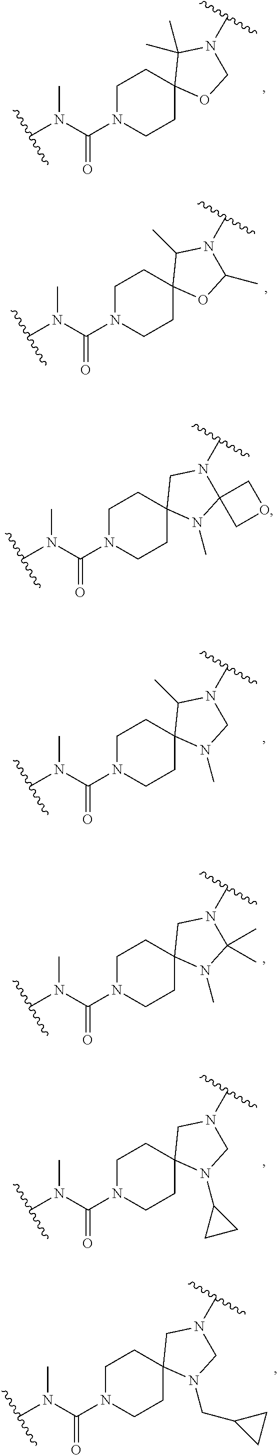

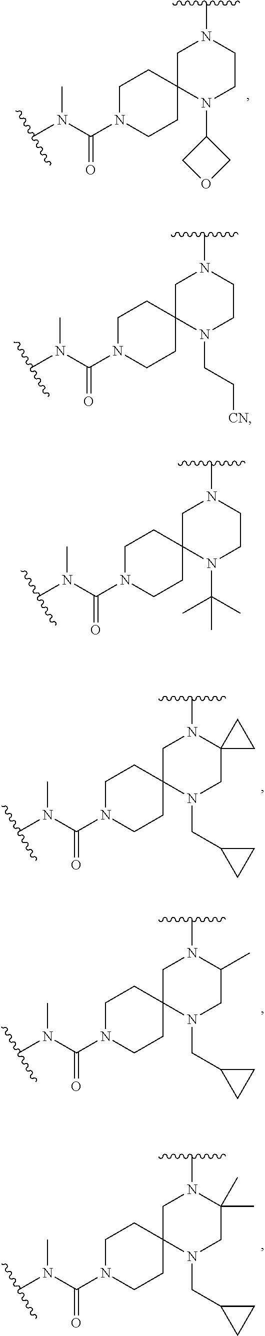 [SMILES: *N1CCN(C2COC2)C2(CCN(C(=O)N(*)C)CC2)C1 ; *N1CCN(CCC#N)C2(CCN(C(=O)N(*)C)CC2)C1 ; *N1CCN(C(C)(C)C)C2(CCN(C(=O)N(*)C)CC2)C1 ; *N(C)C(=O)N1CCC2(CC1)CN(*)C1(CC1)CN2CC1CC1 ; *N(C)C(=O)N1CCC2(CC1)CN(*)C(C)CN2CC1CC1 ; *N(C)C(=O)N1CCC2(CC1)CN(*)C(C)(C)CN2CC1CC1]

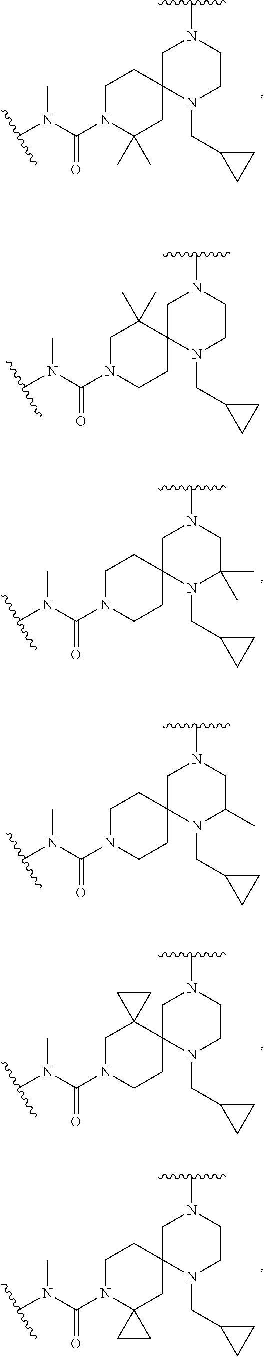 [SMILES: *N1CCN(CC2CC2)C2(CCN(C(=O)N(*)C)C(C)(C)C2)C1 ; *N1CCN(CC2CC2)C2(CCN(C(=O)N(*)C)CC2(C)C)C1 ; *N1CC(C)(C)N(CC2CC2)C2(CCN(C(=O)N(*)C)CC2)C1 ; *N1CC(C)N(CC2CC2)C2(CCN(C(=O)N(*)C)CC2)C1 ; *N1CCN(CC2CC2)C2(CCN(C(=O)N(*)C)CC23CC3)C1 ; *N1CCN(CC2CC2)C2(CCN(C(=O)N(*)C)C3(CC3)C2)C1]

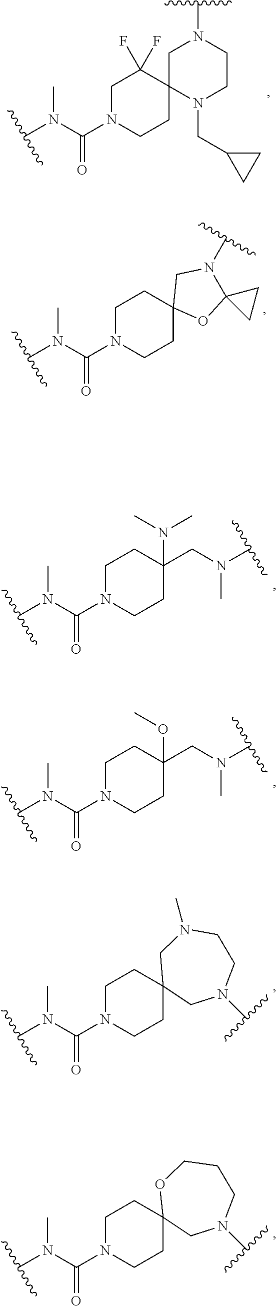 [SMILES: *N1CCN(CC2CC2)C2(CCN(C(=O)N(*)C)CC2(F)F)C1 ; *N(C)C(=O)N1CCC2(CC1)CN(*)C1(CC1)O2 ; *N(C)CC1(N(C)C)CCN(C(=O)N(*)C)CC1 ; *N(C)CC1(OC)CCN(C(=O)N(*)C)CC1 ; *N1CCN(C)CC2(CCN(C(=O)N(*)C)CC2)C1 ; *N1CCCOC2(CCN(C(=O)N(*)C)CC2)C1]

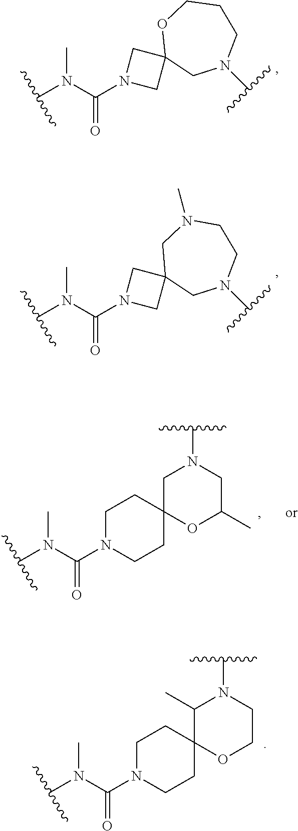 [SMILES: *N1CCCOC2(C1)CN(C(=O)N(*)C)C2 ; *N1CCN(C)CC2(C1)CN(C(=O)N(*)C)C2 ; *N1CC(C)OC2(CCN(C(=O)N(*)C)CC2)C1 ; *N(C)C(=O)N1CCC2(CC1)OCCN(*)C2C]

25. The compound of any one of embodiments 1 to 24, or pharmaceutically acceptable salt thereof, wherein the compound is not a compound of Table 2.
26. The compound of any one of embodiments 1 to 25, or pharmaceutically acceptable salt thereof, having the structure of Formula II-5:

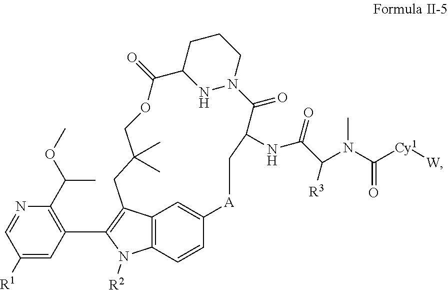

wherein Cy¹ is optionally substituted spirocyclic 8 to 11-membered heterocycloalkylene or optionally substituted bicyclic 7 to 9-membered heterocycloalkylene; and
wherein W comprises a vinyl ketone or a vinyl sulfone.
27. The compound of embodiment 26, or pharmaceutically acceptable salt thereof, wherein Cy¹ is optionally substituted spirocyclic 10 to 11-membered heterocycloalkylene.
28. The compound of embodiment 27, or pharmaceutically acceptable salt thereof, having the structure of Formula II-5a:

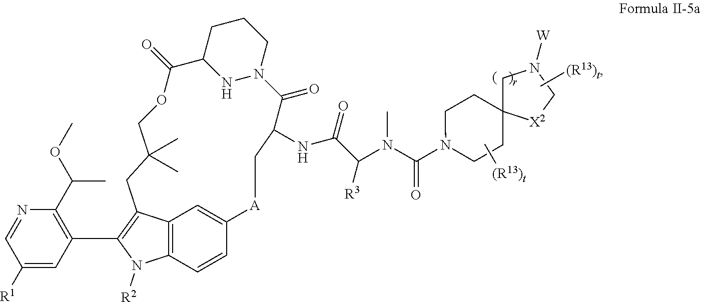

wherein X² is O, C(R¹¹)₂, NR¹², S, or SO₂.
r is 1 or 2;
each t is, independently, 0, 1, or 2;
R¹¹ and R¹² are each, independently, hydrogen, optionally substituted C₁-C₄ alkyl, optionally substituted C₂-C₄ heteroalkyl, or optionally substituted 3 to 5-membered cycloalkyl; and
each R¹³ is, independently, —CH₃.
29. The compound of embodiment 27, or pharmaceutically acceptable salt thereof, having the structure of Formula II-5b:

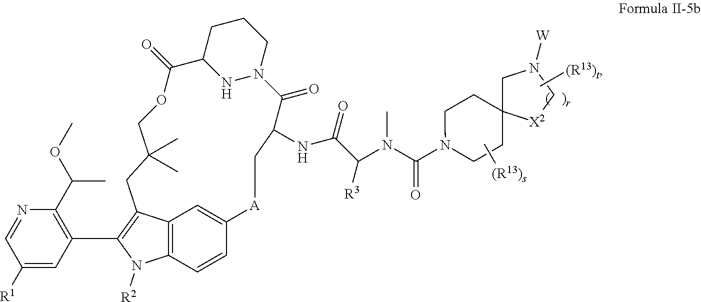

wherein X² is O, C(R¹¹)₂, NR¹², S, or SO₂.
r is 1 or 2;
s and t are each, independently, 0, 1, or 2;
R¹¹ and R¹² are each, independently, hydrogen, optionally substituted C₁-C₄ alkyl, optionally substituted C₂-C₄ heteroalkyl, optionally substituted 3- to 6-membered heterocycloalkyl, or optionally substituted 3 to 5-membered cycloalkyl; and
each R¹³ is, independently, —CH₃, F, or two R¹³ attached to the same atom combine with the atom to which they are attached to form an optionally substituted C₃-C₆ cycloalkyl, or two R¹³ attached to the same atom combine with the atom to which they are attached to form an optionally substituted 3- to 6-membered heterocycloalkyl.
30. The compound of embodiment 29, wherein R¹³ is —CH₃.
31. The compound of embodiment 29, wherein the sum of s and t is 1.
32. The compound of embodiment 29, wherein the sum of s and t is 2.
33. The compound of embodiment 29, wherein s is 0 and tis 1.
34. The compound of embodiment 29, wherein the sum of s and t is 0
35. The compound of embodiment 28 or 29, or pharmaceutically acceptable salt thereof, having the structure of Formula II-5c:

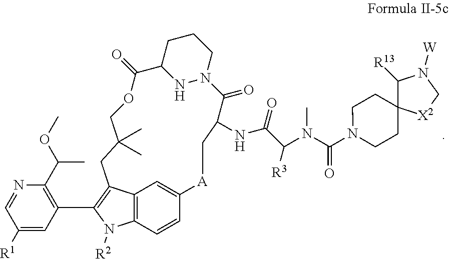

36. The compound of embodiment 28 or 29, or pharmaceutically acceptable salt thereof, having the structure of Formula II-5d:

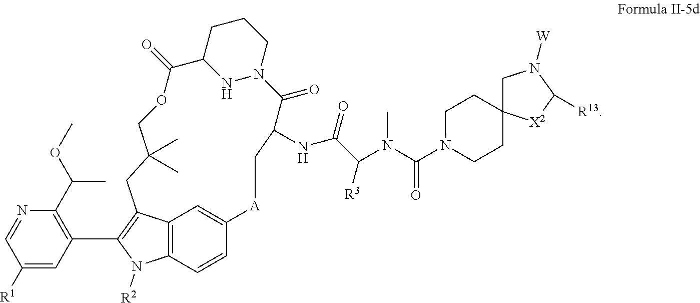

37. The compound of embodiment 28 or 29, or pharmaceutically acceptable salt thereof, having the structure of Formula II-5e:

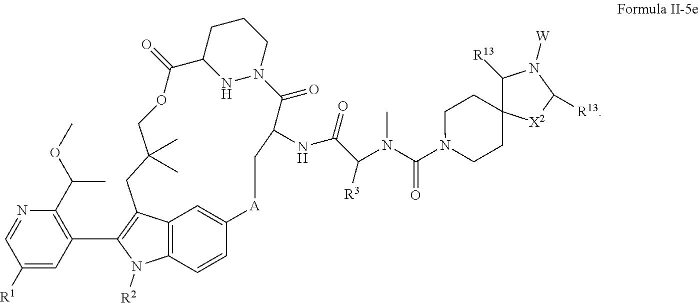

38. The compound of embodiment 28 or 29, or pharmaceutically acceptable salt thereof, wherein r is 1.
39. The compound of embodiment 28 or 29, or pharmaceutically acceptable salt thereof, wherein r is 2.
40. The compound of any one of embodiments 28 to 39, or pharmaceutically acceptable salt thereof, wherein X² is O.
41. The compound of any one of embodiments 28 to 39, or pharmaceutically acceptable salt thereof, wherein X² is S.
42. The compound of any one of embodiments 28 to 39, or pharmaceutically acceptable salt thereof, wherein X² is SO₂.
43. The compound of any one of embodiments 28 to 39, or pharmaceutically acceptable salt thereof, wherein X² is NR¹².
44. The compound of embodiment 43, or pharmaceutically acceptable salt thereof, wherein R¹² is selected from, or a stereoisomer thereof:

### —CH₃,

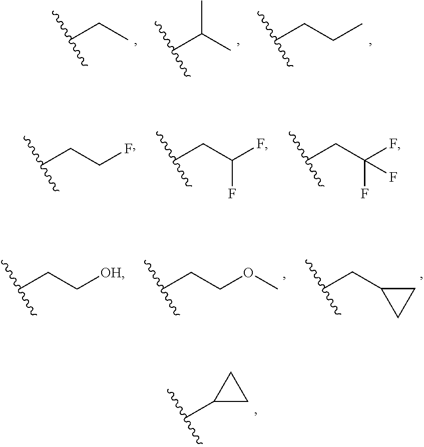

### or —H.

45. The compound of embodiment 43, or a pharmaceutically acceptable salt thereof, wherein R¹² is selected from, or a stereoisomer thereof:

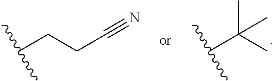

46. The compound of any one of embodiments 28 to 39, or pharmaceutically acceptable salt thereof, wherein X² is C(R¹¹)₂.
47. The compound embodiment 46, or pharmaceutically acceptable salt thereof, wherein each R¹¹ is hydrogen.
48. The compound of any one of embodiments 1 to 47, or pharmaceutically acceptable salt thereof, wherein W is a cross-linking group comprising a vinyl ketone.
49. The compound of embodiment 48, or pharmaceutically acceptable salt thereof, wherein W has the structure of Formula IVa:

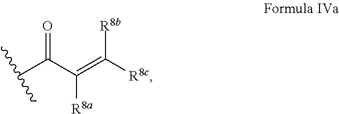

wherein R^{8a}, R^{8b}, and R^{8c} are, independently, hydrogen, —CN, halogen, or —C₁-C₃ alkyl optionally substituted with one or more substituents independently selected from —OH, —O—C₁-C₃ alkyl, —NH₂, —NH(C₁-C₃ alkyl), —N(C₁-C₃ alkyl)₂, or a 4 to 7-membered saturated heterocycloalkyl.
50. The compound of embodiment 49, or pharmaceutically acceptable salt thereof, wherein W is selected from, or a stereoisomer thereof:

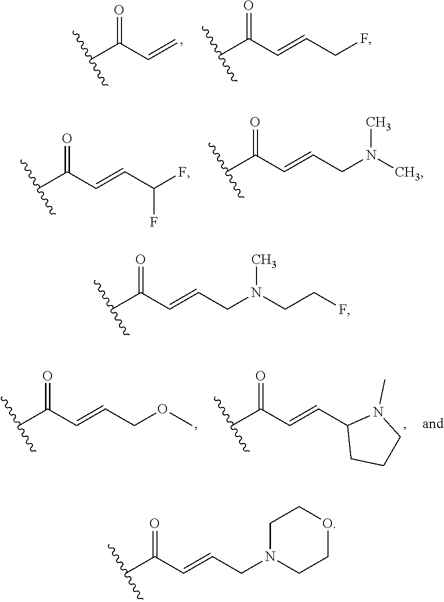

51. The compound of embodiment 49, or pharmaceutically acceptable salt thereof, wherein W is selected from, or a stereoisomer thereof:

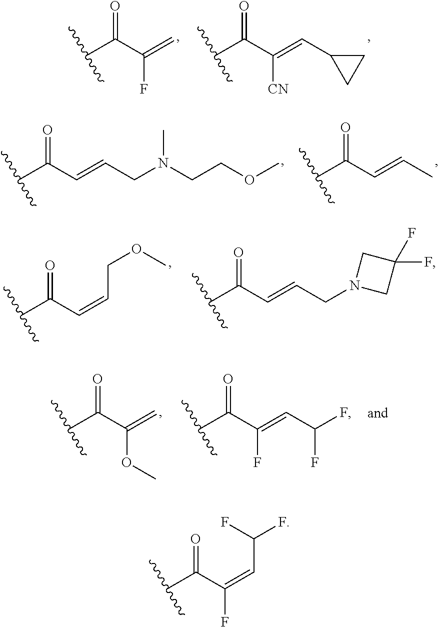

52. The compound of any one of embodiments 1 to 47, or pharmaceutically acceptable salt thereof, wherein W is a cross-linking group comprising a vinyl sulfone.
53. The compound of embodiment 52, or pharmaceutically acceptable salt thereof, wherein W has the structure of Formula IVc:

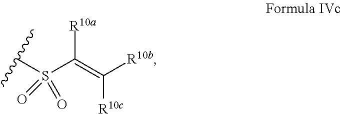

wherein R^{10a}, R^{10b}, and R^{10c} are, independently, hydrogen, —CN, or —C₁-C₃ alkyl optionally substituted with one or more substituents independently selected from —OH, —O—C₁-C₃ alkyl, —NH₂, —NH(C₁-C₃ alkyl), —N(C₁-C₃ alkyl)₂, or a 4 to 7-membered saturated heterocycloalkyl.
54. The compound of embodiment 53, or pharmaceutically acceptable salt thereof, wherein W is:

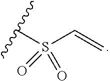 [SMILES: *S(=O)(=O)C=C]

55. The compound of any one of embodiments 1 to 47, or pharmaceutically acceptable salt thereof, wherein W is a cross-linking group comprising an ynone.
56. The compound of embodiment 55, or pharmaceutically acceptable salt thereof, wherein W has the structure of Formula IVb:

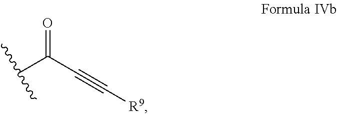

wherein R⁹ is hydrogen, —C₁-C₃ alkyl optionally substituted with one or more substituents independently selected from —OH, —O—C₁-C₃ alkyl, —NH₂, —NH(C₁-C₃ alkyl), —N(C₁-C₃ alkyl)₂, or a 4 to 7-membered saturated cycloalkyl, or a 4 to 7-membered saturated heterocycloalkyl.
57. The compound of embodiment 56, or pharmaceutically acceptable salt thereof, wherein W is selected from:

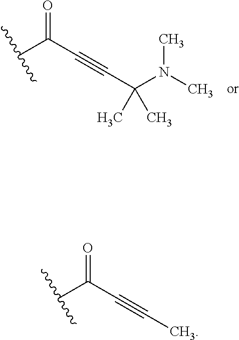 [SMILES: *C(=O)C#CC(C)(C)N(C)C ; *C(=O)C#CC]

58. The compound of embodiment 56 or 57, or pharmaceutically acceptable salt thereof, having the structure of Formula II-6:

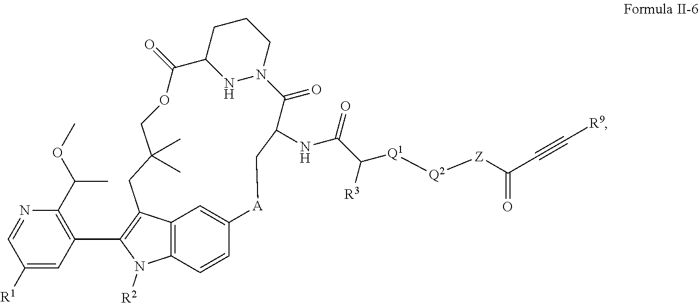

wherein Q¹ is CH₂, NR^{N}, or O;
Q² is CO, NR^{N}, or O; and
Z is optionally substituted 3 to 6-membered heterocycloalkylene or optionally substituted 5 to 10-membered heteroarylene; or
wherein Q¹-Q²-Z is an optionally substituted 9 to 10-membered spirocyclic heterocycloalkylene.
59. The compound of any one of embodiments 56 to 58, or pharmaceutically acceptable salt thereof, having the structure of Formula II-6a:

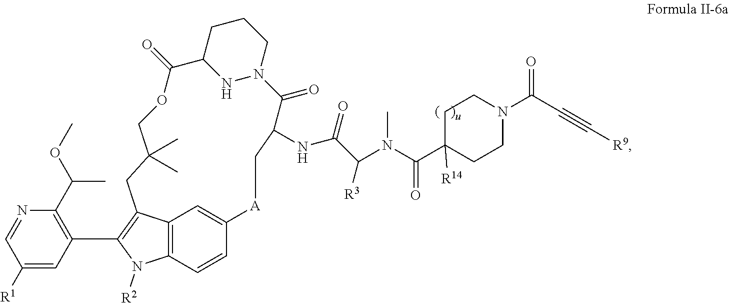

wherein R¹⁴ is fluoro, hydrogen, or C₁-C₃ alkyl; and
u is 0 or 1.
60. The compound of embodiment 59, or pharmaceutically acceptable salt thereof, wherein R¹⁴ is fluoro and u is 1.
61. The compound of embodiment 59, or pharmaceutically acceptable salt thereof, wherein R¹⁴ is hydrogen and u is 0.
62. The compound of any one of embodiments 56 to 59, or pharmaceutically acceptable salt thereof, having the structure of Formula II-6b:

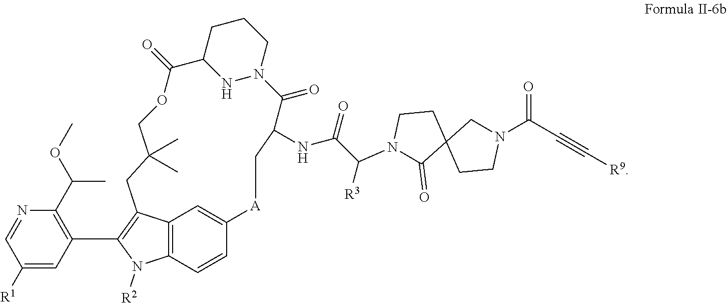

63. The compound of any one of embodiments 56 to 59, or pharmaceutically acceptable salt thereof, having the structure of Formula II-6c:

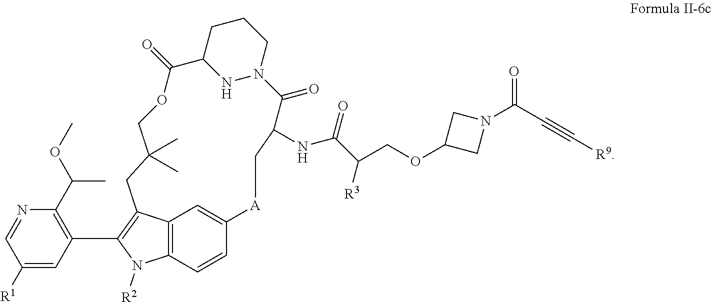

64. A compound, or a pharmaceutically acceptable salt thereof, selected from Table 1.
65. A pharmaceutical composition comprising a compound of any one of embodiments 1 to 64, or a pharmaceutically acceptable salt thereof, and a pharmaceutically acceptable excipient.
66. A conjugate, or salt thereof, comprising the structure of Formula V:

M-L-P Formula V,
wherein L is a linker;
P is a monovalent organic moiety; and
M has the structure of Formula VIa:

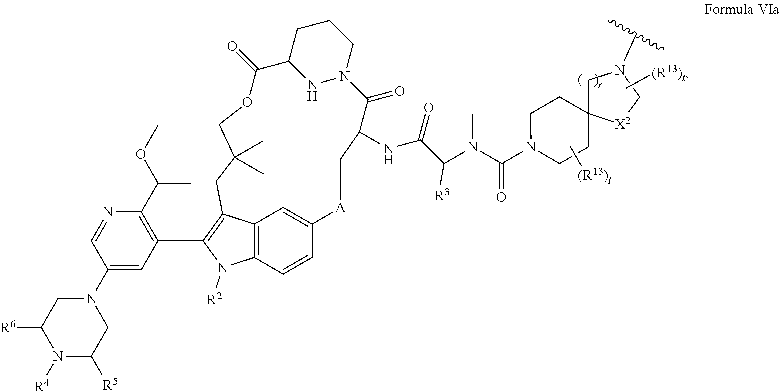

wherein A is optionally substituted 3 to 6-membered heterocycloalkylene, optionally substituted 3 to 6-membered cycloalkylene, optionally substituted 6-membered arylene, or optionally substituted 5 to 10-membered heteroarylene;
R² is optionally substituted C₁-C₆ alkyl;
R³ is optionally substituted C₁-C₆ alkyl or optionally substituted C₁-C₃ heteroalkyl;
X² is O, C(R¹¹)₂, NR¹², S, or SO₂;
r is 1 or 2;
each t is, independently, 0, 1, or 2;
R¹¹ and R¹² are each, independently, hydrogen, optionally substituted C₁-C₄ alkyl, optionally substituted C₂-C₄ heteroalkyl, or optionally substituted 3 to 5-membered cycloalkyl;
each R¹³ is, independently, —CH₃; and
R⁴, R⁵, and R⁶ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered heterocycloalkyl; or
R⁴ and R⁵ combine with the atoms to which they are attached to form an optionally substituted 3 to 8-membered cycloalkyl or an optionally substituted 3 to 8-membered heterocycloalkyl; or
R⁴ and R⁶ combine with the atoms to which they are attached to form an optionally substituted 3 to 8-membered cycloalkyl or an optionally substituted 3 to 8-membered heterocycloalkyl.
67. A conjugate, or salt thereof, comprising the structure of Formula V:

M-L-P Formula V,
wherein L is a linker;
P is a monovalent organic moiety; and
M has the structure of Formula VIb:

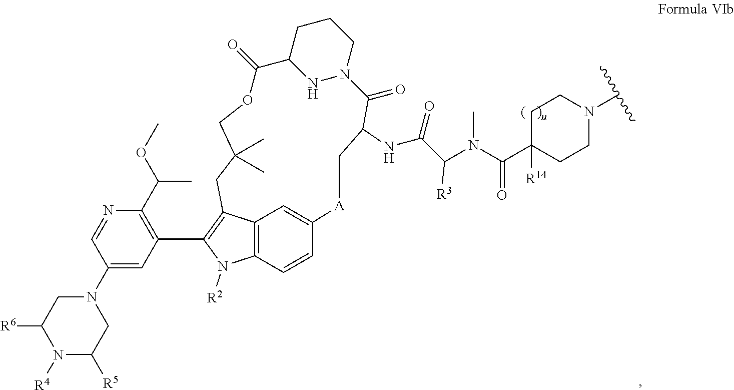

wherein A is optionally substituted 3 to 6-membered heterocycloalkylene, optionally substituted 3 to 6-membered cycloalkylene, optionally substituted 6-membered arylene, or optionally substituted 5 to 10-membered heteroarylene;
R² is optionally substituted C₁-C₆ alkyl;
R³ is optionally substituted C₁-C₆ alkyl or optionally substituted C₁-C₃ heteroalkyl;
R¹⁴ is fluoro, hydrogen, or C₁-C₃ alkyl;
u is 0 or 1; and
R⁴, R⁵, and R⁶ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered heterocycloalkyl; or
R⁴ and R⁵ combine with the atoms to which they are attached to form an optionally substituted 3 to 8-membered cycloalkyl or an optionally substituted 3 to 8-membered heterocycloalkyl; or
R⁴ and R⁶ combine with the atoms to which they are attached to form an optionally substituted 3 to 8-membered cycloalkyl or an optionally substituted 3 to 8-membered heterocycloalkyl.
68. A conjugate, or salt thereof, comprising the structure of Formula V:

M-L-P Formula V,
wherein L is a linker;
P is a monovalent organic moiety; and
M has the structure of Formula VIc:

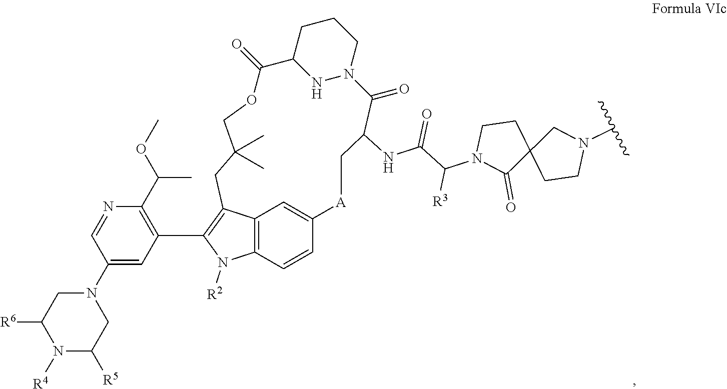

wherein A is optionally substituted 3 to 6-membered heterocycloalkylene, optionally substituted 3 to 6-membered cycloalkylene, optionally substituted 6-membered arylene, or optionally substituted 5 to 10-membered heteroarylene;
R² is optionally substituted C₁-C₆ alkyl;
R³ is optionally substituted C₁-C₆ alkyl or optionally substituted C₁-C₃ heteroalkyl; and
R⁴, R⁵, and R⁶ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered heterocycloalkyl; or
R⁴ and R⁵ combine with the atoms to which they are attached to form an optionally substituted 3 to 8-membered cycloalkyl or an optionally substituted 3 to 8-membered heterocycloalkyl; or
R⁴ and R⁶ combine with the atoms to which they are attached to form an optionally substituted 3 to 8-membered cycloalkyl or an optionally substituted 3 to 8-membered heterocycloalkyl.
69. A conjugate, or salt thereof, comprising the structure of Formula V:

M-L-P Formula V,
wherein L is a linker;
P is a monovalent organic moiety; and
M has the structure of Formula VId:

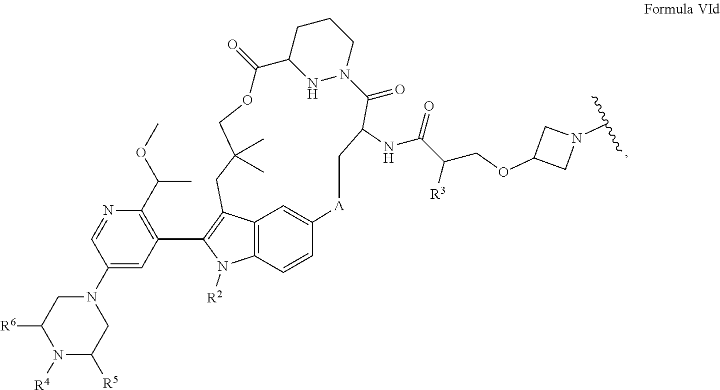

wherein A is optionally substituted 3 to 6-membered heterocycloalkylene, optionally substituted 3 to 6-membered cycloalkylene, optionally substituted 6-membered arylene, or optionally substituted 5 to 10-membered heteroarylene;
R² is optionally substituted C₁-C₆ alkyl;
R³ is optionally substituted C₁-C₆ alkyl or optionally substituted C₁-C₃ heteroalkyl; and
R⁴, R⁵, and R⁶ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered heterocycloalkyl; or
R⁴ and R⁵ combine with the atoms to which they are attached to form an optionally substituted 3 to 8-membered cycloalkyl or an optionally substituted 3 to 8-membered heterocycloalkyl; or
R⁴ and R⁶ combine with the atoms to which they are attached to form an optionally substituted 3 to 8-membered cycloalkyl or an optionally substituted 3 to 8-membered heterocycloalkyl.
70. The conjugate of any one of embodiments 66 to 69, or salt thereof, wherein the monovalent organic moiety is a protein.
71. The conjugate of embodiment 70, or salt thereof, wherein the protein is a Ras protein.
72. The conjugate of embodiment 71, or salt thereof, wherein the Ras protein is K-Ras G12C, K-Ras G13C, H-Ras G12C, H-Ras G13C, N-Ras G12C, or N-Ras G13C.
73. The conjugate of embodiment 72, wherein the Ras protein is K-Ras G13C.
74. The conjugate of any one of embodiments 66 to 73, or a salt thereof, wherein the linker is bound to the monovalent organic moiety through a bond to a sulfhydryl group of an amino acid residue of the monovalent organic moiety.
75. A method of treating cancer in a subject in need thereof, the method comprising administering to the subject a therapeutically effective amount of a compound of any one of embodiments 1 to 64, or a pharmaceutically acceptable salt thereof, or a pharmaceutical composition of embodiment 65.
76. The method of embodiment 75, wherein the cancer is pancreatic cancer, colorectal cancer, non-small cell lung cancer, or endometrial cancer.
77. The method of embodiment 75 or 76, wherein the cancer comprises a Ras mutation.
78. The method of embodiment 77, wherein the Ras mutation is K-Ras G12C, K-Ras G13C, H-Ras G12C, H-Ras G13C, N-Ras G12C, or N-Ras G13C.
79. The method of embodiment 78, wherein the Ras mutation is K-Ras G13C.
80. A method of treating a Ras protein-related disorder in a subject in need thereof, the method comprising administering to the subject a therapeutically effective amount of a compound of any one of embodiments 1 to 64, or a pharmaceutically acceptable salt thereof, or a pharmaceutical composition of embodiment 65.
81. A method of inhibiting a Ras protein in a cell, the method comprising contacting the cell with an effective amount of a compound of any one of embodiments 1 to 64, or a pharmaceutically acceptable salt thereof, or a pharmaceutical composition of embodiment 65.
82. The method of embodiment 80 or 81, wherein the Ras protein is K-Ras G12C, K-Ras G13C, H-Ras G12C, H-Ras G13C, N-Ras G12C, or N-Ras G13C.
83. The method of embodiment 82, wherein the Ras protein is K-Ras G13C.
84. The method of any one of embodiments 81 to 83, wherein the cell is a cancer cell.
85. The method of embodiment 84, wherein the cancer cell is a pancreatic cancer cell, a colorectal cancer cell, a non-small cell lung cancer cell, or an endometrial cancer cell.
86. The method or use of any one of embodiments 75 to 85, wherein the method or use further comprises administering an additional anti-cancer therapy.
87. The method of embodiment 86, wherein the additional anti-cancer therapy is an EGFR inhibitor, a second Ras inhibitor, a SHP2 inhibitor, a SOS1 inhibitor, a Raf inhibitor, a MEK inhibitor, an ERK inhibitor, a PI3K inhibitor, a PTEN inhibitor, an AKT inhibitor, an mTORC1 inhibitor, a BRAF inhibitor, a PD-L1 inhibitor, a PD-1 inhibitor, a CDK4/6 inhibitor, a HER2 inhibitor, or a combination thereof.
88. The method of embodiment 86 or 87, wherein the additional anti-cancer therapy is a SHP2 inhibitor.

### EXAMPLES

The disclosure is further illustrated by the following examples and synthesis examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure or scope of the appended claims.

### CHEMICAL SYNTHESES

Definitions used in the following examples and elsewhere herein are:
CH₂Cl₂, DCM Methylene chloride, Dichloromethane
CH₃CN, MeCN Acetonitrile
CuI Copper (I) iodide
DIPEA Diisopropylethyl amine
DMF N,N-Dimethylformamide
EtOAc Ethyl acetate
h hour
H₂O Water
HCl Hydrochloric acid
K₃PO₄ Potassium phosphate (tribasic)
MeOH Methanol
Na₂SO₄ Sodium sulfate
NMP N-methyl pyrrolidone
Pd(dppf)Cl₂ [1,1′-Bis(diphenylphosphino)ferrocene]dichloropalladium(II)

### Instrumentation

Mass spectrometry data collection took place with a Shimadzu LCMS-2020, an Agilent 1260LC-6120/6125MSD, a Shimadzu LCMS-2010EV, or a Waters Acquity UPLC, with either a QDa detector or SQ Detector 2. Samples were injected in their liquid phase onto a C-18 reverse phase. The compounds were eluted from the column using an acetonitrile gradient and fed into the mass analyzer. Initial data analysis took place with either Agilent ChemStation, Shimadzu LabSolutions, or Waters MassLynx. NMR data was collected with either a Bruker AVANCE III HD 400 MHZ, a Bruker Ascend 500 MHz instrument, or a Varian 400 MHz, and the raw data was analyzed with either TopSpin or Mestrelab Mnova.

### SYNTHESIS OF INTERMEDIATES

### Intermediate 1. Synthesis of 3-(5-bromo-1-ethyl-2-[2-[(1S)-1-methoxyethyl]pyridin-3-yl]indol-3-yl)-2,2-dimethylpropan-1-ol

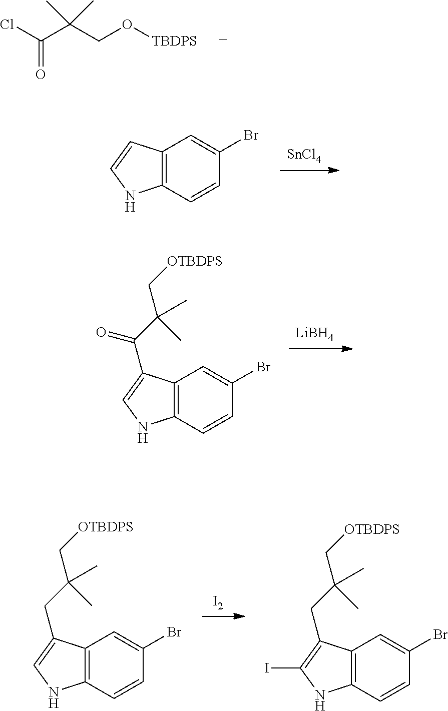

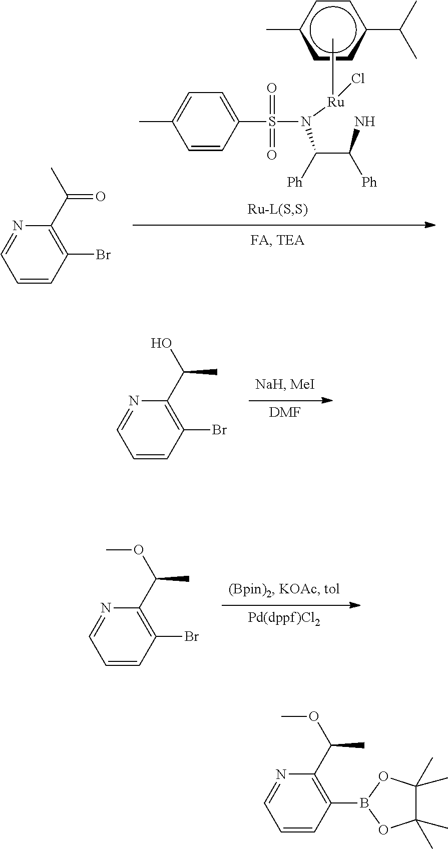

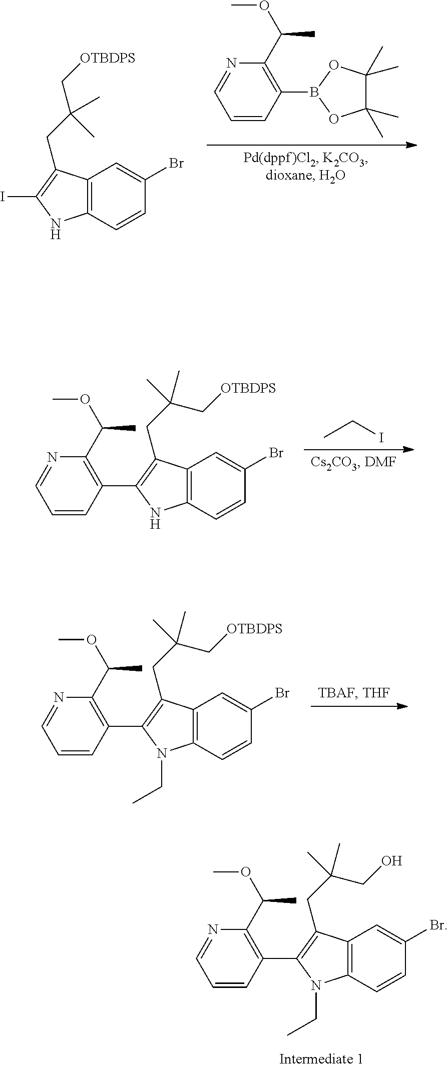

Step 1. To a mixture of 3-((tert-butyldiphenylsilyl)oxy)-2,2-dimethylpropanoyl chloride (65 g, 137 mmol, crude) in DCM (120 mL) at 0° C. under an atmosphere of N₂ was added 1M SnCl₄ in DCM (137 mL, 137 mmol) slowly. The mixture was stirred at 0° C. for 30 min, then a solution of 5-bromo-1H-indole (26.8 g, 137 mmol) in DCM (40 mL) was added dropwise. The mixture was stirred at 0° C. for 45 min, then diluted with EtOAc (300 mL), washed with brine (100 mL×4), dried over Na₂SO₄, and filtered. The filtrate was concentrated under reduced pressure and the residue was purified by silica gel column chromatography to give 1-(5-bromo-1H-indol-3-yl)-3-((tert-butyldiphenylsilyl)oxy)-2,2-dimethylpropan-1-one (55 g, 75% yield). LCMS (ESI): m/z [M+Na] calc'd for C₂₉H₃₂BrNO₂SiNa 556.1; found 556.3.
Step 2. To a mixture of 1-(5-bromo-1H-indol-3-yl)-3-((tert-butyldiphenylsilyl)oxy)-2,2-dimethylpropan-1-one (50 g, 93.6 mmol) in THF (100 mL) at 0° C. under an atmosphere of N₂ was added LiBH₄ (6.1 g, 281 mmol). The mixture was heated to 60° C. and stirred for 20 h, then MeOH (10 mL) and EtOAc (100 mL) were added, and the mixture washed with brine (50 mL), dried over Na₂SO₄, filtered, and the filtrate concentrated under reduced pressure. The residue was diluted with DCM (50 mL), cooled to 10° C. and diludine (9.5 g, 37.4 mmol) and TsOH·H₂O (890 mg, 4.7 mmol) added. The mixture was stirred at 10° C. for 2 h, filtered, the filtrate concentrated under reduced pressure and the residue was purified by silica gel column chromatography to give 1-(5-bromo-1H-indol-3-yl)-3-((tert-butyldiphenylsilyl)oxy)-2,2-dimethylpropan-1-one (41 g, 84% yield). LCMS (ESI): m/z [M+H] calc'd for C₂₉H₃₄BrNOSi 519.2; found 520.1; ¹H NMR (400 MHz, CDCl₃) δ 7.96 (s, 1H), 7.75-7.68 (m, 5H), 7.46-7.35 (m, 6H), 7.23-7.19 (m, 2H), 6.87 (d, J=2.1 Hz, 1H), 3.40 (s, 2H), 2.72 (s, 2H), 1.14 (s, 9H), 0.89 (s, 6H).
Step 3. To a mixture of 1-(5-bromo-1H-indol-3-yl)-3-((tert-butyldiphenylsilyl)oxy)-2,2-dimethylpropan-1-one (1.5 g, 2.9 mmol) and 12 (731 mg, 2.9 mmol) in THF (15 mL) at rt was added AgOTf (888 mg, 3.5 mmol). The mixture was stirred at rt for 2 h, then diluted with EtOAc (200 mL) and washed with saturated Na₂S₂O₃ (100 mL), dried over anhydrous Na₂SO₄, and filtered. The filtrate was concentrated under reduced pressure and the residue was purified by silica gel column chromatography to give 5-bromo-3-(3-((tert-butyldiphenylsilyl)oxy)-2,2-dimethylpropyl)-2-iodo-1H-indole (900 mg, 72% yield) as a solid. ¹H NMR (400 MHz, DMSO-d₆) δ 11.70 (s, 1H), 7.68 (d, J=1.3 Hz, 1H), 7.64-7.62 (m, 4H), 7.46-7.43 (m, 6H), 7.24-7.22 (d, 1H), 7.14-7.12 (dd, J=8.6, 1.6 Hz, 1H), 3.48 (s, 2H), 2.63 (s, 2H), 1.08 (s, 9H), 0.88 (s, 6H).
Step 4. To a stirred mixture of HCOOH (66.3 g, 1.44 mol) in TEA (728 g, 7.2 mol) at 0° C. under an atmosphere of Ar was added (4S,5S)-2-chloro-2-methyl-1-(4-methylbenzenesulfonyl)-4,5-diphenyl-1,3-diaza-2-ruthenacyclopentane cymene (3.9 g, 6.0 mmol) portion-wise. The mixture was heated to 40° C. and stirred for 15 min, then cooled to rt and 1-(3-bromopyridin-2-yl) ethanone (120 g, 600 mmol) added in portions. The mixture was heated to 40° C. and stirred for an additional 2 h, then the solvent was concentrated under reduced pressure. Brine (2 L) was added to the residue, the mixture was extracted with EtOAc (4×700 mL), dried over anhydrous Na₂SO₄, and filtered. The filtrate was concentrated under reduced pressure and the residue was purified by silica gel column chromatography to give (1S)-1-(3-bromopyridin-2-yl) ethanol (100 g, 74% yield) as an oil. LCMS (ESI): m/z [M+H] calc'd for C₇H₈BrNO 201.1; found 201.9.
Step 5. To a stirred mixture of (1S)-1-(3-bromopyridin-2-yl) ethanol (100 g, 495 mmol) in DMF (1 L) at 0° C. was added NaH, 60% dispersion in oil (14.25 g, 594 mmol) in portions. The mixture was stirred at 0° C. for 1 h. Mel (140.5 g, 990 mmol) was added dropwise at 0° C. and the mixture was allowed to warm to rt and stirred for 2 h. The mixture was cooled to 0° C. and saturated NH₄Cl (5 L) was added. The mixture was extracted with EtOAc (3×1.5 L), dried over anhydrous Na₂SO₄, and filtered. The filtrate was concentrated under reduced pressure and the residue was purified by silica gel column chromatography to give 3-bromo-2-[(1S)-1-methoxyethyl]pyridine (90 g, 75% yield) as an oil. LCMS (ESI): m/z [M+H] calc'd for C₈H₁₀BrNO 215.0; found 215.9.
Step 6. To a stirred mixture of 3-bromo-2-[(1S)-1-methoxyethyl]pyridine (90 g, 417 mmol) and Pd(dppf)Cl₂ (30.5 g, 41.7 mmol) in toluene (900 mL) at rt under an atmosphere of Ar was added bis(pinacolato)diboron (127 g, 500 mmol) and KOAc (81.8 g, 833 mmol) in portions. The mixture was heated to 100° C. and stirred for 3 h. The filtrate was concentrated under reduced pressure and the residue was purified by Al₂O₃ column chromatography to give 2-[(1S)-1-methoxyethyl]-3-(4,4,5,5-tetramethyl-1,3,2-dioxaborolan-2-yl)pyridine (100 g, 63% yield) as a semi-solid. LCMS (ESI): m/z [M+H] calc'd for C₁₄H₂₂BNO₃ 263.2; found 264.1.
Step 7. To a stirred mixture of 5-bromo-3-[3-[(tert-butyldiphenylsilyl)oxy]-2,2-dimethylpropyl]-2-iodo-1H-indole (140 g, 217 mmol) and 2-[(1S)-1-methoxyethyl]-3-(4,4,5,5-tetramethyl-1,3,2-dioxaborolan-2-yl)pyridine (100 g, 380 mmol) in 1,4-dioxane (1.4 L) at rt under an atmosphere of Ar was added K₂CO₃ (74.8 g, 541 mmol), Pd(dppf)Cl₂ (15.9 g, 21.7 mmol), and H₂O (280 mL) in portions. The mixture was heated to 85° C. and stirred for 4 h, then cooled, H₂O (5 L) added, and the mixture extracted with EtOAc (3×2 L). The combined organic layers were washed with brine (2×1 L), dried over anhydrous Na₂SO₄, and filtered. The filtrate was concentrated under reduced pressure and the residue was purified by silica gel column chromatography to give 5-bromo-3-[3-[(tert-butyldiphenylsilyl)oxy]-2,2-dimethylpropyl]-2-[2-[(1S)-1-methoxyethyl]pyridin-3-yl]-1H-indole (71 g, 45% yield) as a solid. LCMS (ESI): m/z [M+H] calc'd for C₃₇H₄₃BrN₂O₂Si 654.2; found 655.1.
Step 8. To a stirred mixture of 5-bromo-3-[3-[(tert-butyldiphenylsilyl)oxy]-2,2-dimethylpropyl]-2-[2-[(1S)-1-methoxyethyl]pyridin-3-yl]-1H-indole (71 g, 108 mmol) in DMF (0.8 L) at 0° C. under an atmosphere of N₂ was added Cs₂CO₃ (70.6 g, 217 mmol) and Etl (33.8 g, 217 mmol) in portions. The mixture was warmed to rt and stirred for 16 h then H₂O (4 L) added, and the mixture extracted with EtOAc (3×1.5 L). The combined organic layers were washed with brine (2×1 L), dried over anhydrous Na₂SO₄, and filtered. The filtrate was concentrated under reduced pressure and the residue was purified by silica gel column chromatography to give 5-bromo-3-[3-[(tert-butyldiphenylsilyl)oxy]-2,2-dimethylpropyl]-1-ethyl-2-[2-[(1S)-1-methoxyethyl]pyridin-3-yl]indole (66 g, 80% yield) as an oil. LCMS (ESI): m/z [M+H] calc'd for C₃₉H₄₇BrN₂O₂Si 682.3; found 683.3.
Step 9. To a stirred mixture of TBAF (172.6 g, 660 mmol) in THF (660 mL) at rt under an atmosphere of N₂ was added 5-bromo-3-[3-[(tert-butyldiphenylsilyl)oxy]-2,2-dimethylpropyl]-1-ethyl-2-[2-[(1S)-1-methoxyethyl]pyridin-3-yl]indole (66 g, 97 mmol) in portions. The mixture was heated to 50° C. and stirred for 16 h, cooled, diluted with H₂O (5 L), and extracted with EtOAc (3×1.5 L). The combined organic layers were washed with brine (2×1 L), dried over anhydrous Na₂SO₄, and filtered. After filtration, the filtrate was concentrated under reduced pressure. The filtrate was concentrated under reduced pressure and the residue was purified by silica gel column chromatography to give 3-(5-bromo-1-ethyl-2-[2-[(1S)-1-methoxyethyl]pyridin-3-yl]indol-3-yl)-2,2-dimethylpropan-1-ol (30 g, 62% yield) as a solid. LCMS (ESI): m/z [M+H] calc'd for C₂₃H₂₉BrN₂O₂ 444.1; found 445.1.

### Intermediate 1. Alternative Synthesis Through Fisher Indole Route

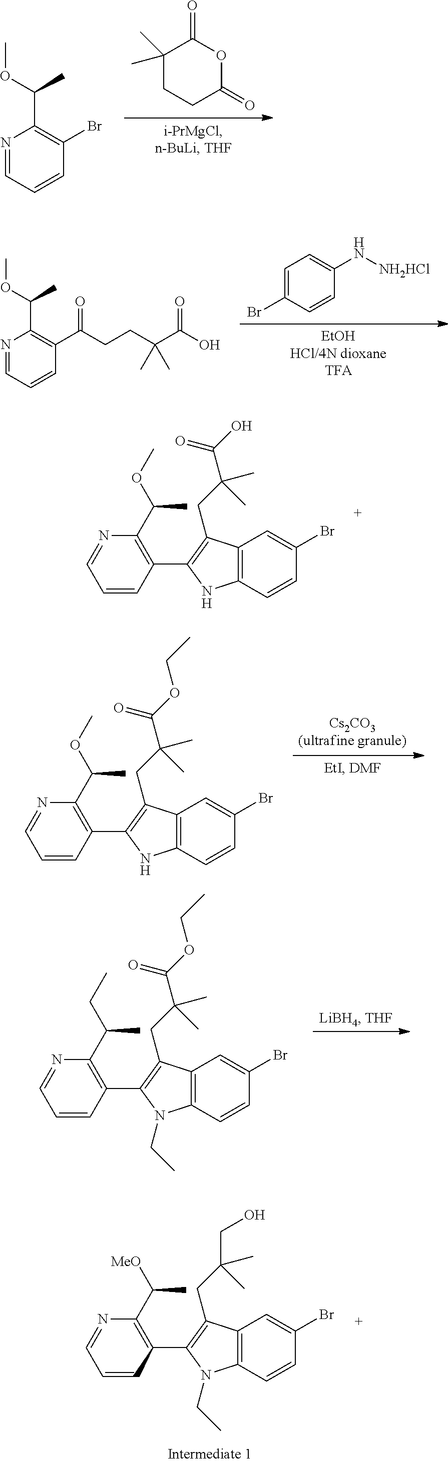

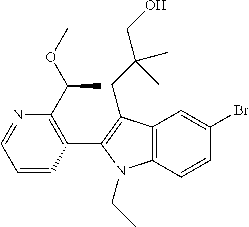

Step 1. To a mixture of i-PrMgCl (2M in in THF, 0.5 L) at −10° C. under an atmosphere of N₂ was added n-BuLi, 2.5 M in hexane (333 mL, 833 mmol) dropwise over 15 min. The mixture was stirred for 30 min at −10° C. then 3-bromo-2-[(1S)-1-methoxyethyl]pyridine (180 g, 833 mmol) in THF (0.5 L) added dropwise over 30 min at −10° C. The resulting mixture was warmed to −5° C. and stirred for 1 h, then 3,3-dimethyloxane-2,6-dione (118 g, 833 mmol) in THF (1.2 L) was added dropwise over 30 min at −5° C. The mixture was warmed to 0° C. and stirred for 1.5 h, then quenched with the addition of pre-cooled 4M HCl in 1,4-dioxane (0.6 L) at 0° C. to adjust pH ˜5. The mixture was diluted with ice-water (3 L) and extracted with EtOAc (3×2.5 L). The combined organic layers were dried over anhydrous Na₂SO₄, filtered, the filtrate was concentrated under reduced pressure, and the residue was purified by silica gel column chromatography to give 5-[2-[(1S)-1-methoxyethyl]pyridin-3-yl]-2,2-dimethyl-5-oxopentanoic acid (87 g, 34% yield) as a solid. LCMS (ESI): m/z [M+H] calc'd for C₁₅H₂₁NO₄ 279.2; found 280.1.
Step 2. To a mixture of 5-[2-[(1S)-1-methoxyethyl]pyridin-3-yl]-2,2-dimethyl-5-oxopentanoic acid (78 g, 279 mmol) in EtOH (0.78 L) at rt under an atmosphere of N₂ was added (4-bromophenyl) hydrazine HCl salt (68.7 g, 307 mmol) in portions. The mixture was heated to 85° C. and stirred for 2 h, cooled to rt, then 4M HCl in 1,4-dioxane (69.8 mL, 279 mmol) added dropwise. The mixture was heated to 85° C. and stirred for an additional 3 h, then concentrated under reduced pressure, and the residue was dissolved in TFA (0.78 L). The mixture was heated to 60° C. and stirred for 1.5 h, concentrated under reduced pressure, and the residue adjusted to PH ˜5 with saturated NaHCO₃, then extracted with EtOAc (3×1.5 L). The combined organic layers were dried over anhydrous Na₂SO₄, filtered, the filtrate concentrated under reduced pressure, and the residue was purified by silica gel column chromatography to give 3-(5-bromo-2-[2-[(1S)-1-methoxyethyl]pyridin-3-yl]-1H-indol-3-yl)-2,2-dimethylpropanoic acid and ethyl(S)-3-(5-bromo-2-(2-(1-methoxyethyl)pyridin-3-yl)-1H-indol-3-yl)-2,2-dimethylpropanoate (78 g, crude). LCMS (ESI): m/z [M+H] calc'd for C₂₁H₂₃BrN₂O₃ 430.1 and C₂₃H₂₇BrN₂O₃ 458.1; found 431.1 and 459.1.
Step 3. To a mixture of 3-(5-bromo-2-[2-[(1S)-1-methoxyethyl]pyridin-3-yl]-1H-indol-3-yl)-2,2-dimethylpropanoic acid and ethyl(S)-3-(5-bromo-2-(2-(1-methoxyethyl)pyridin-3-yl)-1H-indol-3-yl)-2,2-dimethylpropanoate (198 g, 459 mmol) in DMF (1.8 L) at 0° C. under an atmosphere of N₂ was added Cs₂CO₃ (449 g, 1.38 mol) in portions. Etl (215 g, 1.38 mmol) in DMF (200 mL) was then added dropwise at 0° C. The mixture was warmed to rt and stirred for 4 h then diluted with brine (5 L) and extracted with EtOAc (3×2.5 L). The combined organic layers were washed with brine (2×1.5 L), dried over anhydrous Na₂SO₄, and filtered. The filtrate was concentrated under reduced pressure and the residue was purified by silica gel column chromatography to give ethyl 3-(5-bromo-1-ethyl-2-[2-[(1S)-1-methoxyethyl]pyridin-3-yl]indol-3-yl)-2,2-dimethylpropanoate (160 g, 57% yield) as a solid. LCMS (ESI): m/z [M+H] calc'd for C₂₅H₃₁BrN₂O₃ 486.2; found 487.2.
Step 4. To a mixture of ethyl 3-(5-bromo-1-ethyl-2-[2-[(1S)-1-methoxyethyl]pyridin-3-yl]indol-3-yl)-2,2-dimethylpropanoate (160 g, 328 mmol) in THF (1.6 L) at 0° C. under an atmosphere of N₂ was added LiBH₄ (28.6 g, 1.3 mol). The mixture was heated to 60° C. for 16 h, cooled, and quenched with pre-cooled (0° C.) aqueous NH₄Cl (5 L). The mixture was extracted with EtOAc (3×2 L), and the combined organic layers were washed with brine (2×1 L), dried over anhydrous Na₂SO₄, and filtered. The filtrate was concentrated under reduced pressure and the residue was purified by silica gel column chromatography to give to two atropisomers (as single atropisomers) of 3-(5-bromo-1-ethyl-2-(2-((S)-1-methoxyethyl)pyridin-3-yl)-1H-indol-3-yl)-2,2-dimethylpropan-1-ol (60 g, 38% yield) and (40 g, 26% yield) both as solids. LCMS (ESI): m/z [M+H] calc'd for C₂₃H₂₉BrN₂O₂ 444.1; found 445.2.

### Intermediate 2. Synthesis of tert-butyl ((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)- pyridazinacycloundecaphane-4-yl)carbamate

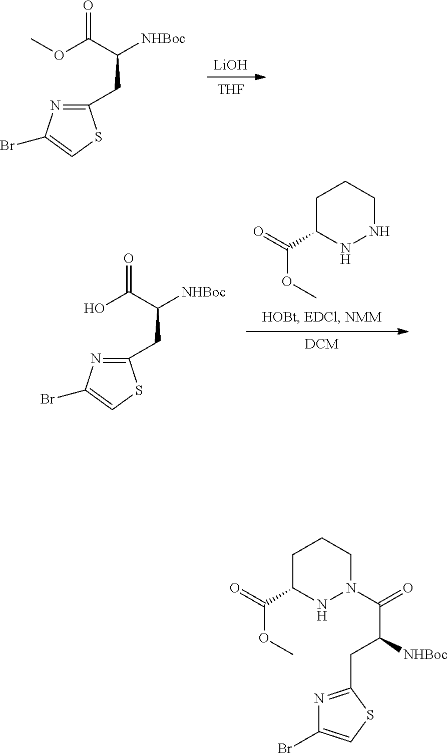

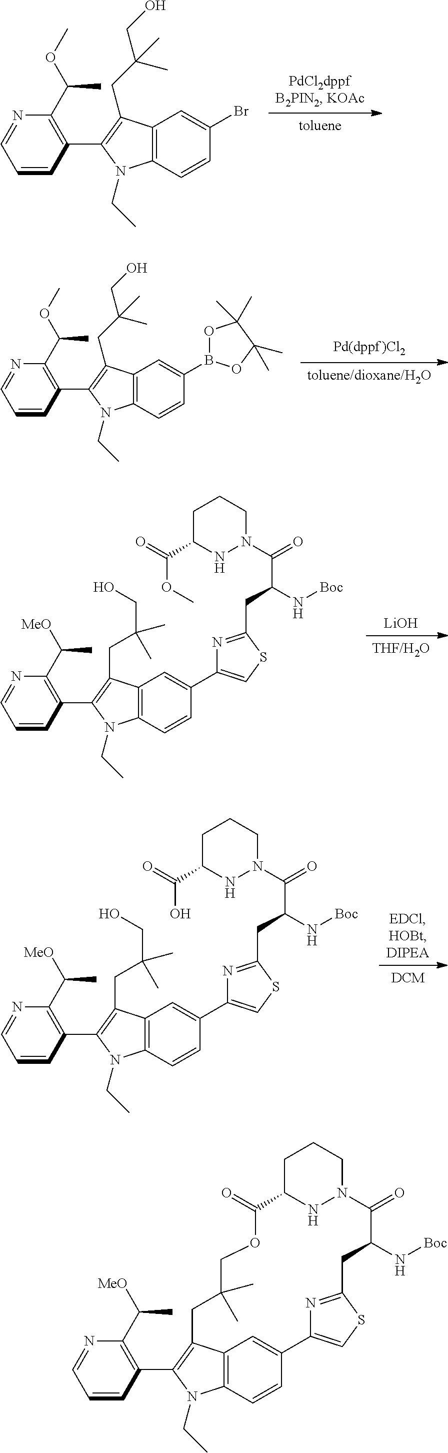

Step 1. To a solution of methyl (2S)-3-(4-bromo-1,3-thiazol-2-yl)-2-[(tert-butoxycarbonyl)amino]propanoate (110 g, 301.2 mmol) in THF (500 mL) and H₂O (200 mL) at room temperature was added LiOH (21.64 g, 903.6 mmol). The resulting solution was stirred for 1 h and was then concentrated under reduced pressure. The resulting residue was adjusted to pH 6 with 1 M HCl and then extracted with DCM (3×500 mL). The combined organic layers were, dried over Na₂SO₄, filtered, and concentrated under reduced pressure to afford the desired product (108 g, crude). LCMS (ESI) m/z: [M+H] calcd for C₁₁H₁₅BrN₂O₄S: 351.00; found 351.0.
Step 2. To a solution of (S)-3-(4-bromothiazol-2-yl)-2-((tert-butoxycarbonyl)amino)propanoic acid (70 g, 199.3 mmol) in DCM (500 mL) at 0° C. was added methyl (3S)-1,2-diazinane-3-carboxylate bis(trifluoroacetic acid) salt (111.28 g, 298.96 mmol), NMM (219.12 mL, 1993.0 mmol), EDCl (76.41 g, 398.6 mmol) and HOBt (5.39 g, 39.89 mmol). The resulting solution was warmed to room temperature and stirred for 1 h. The reaction was then quenched with H₂O (500 mL) and was extracted with EtOAc (3×500 mL). The combined organic layers were dried over Na₂SO₄, filtered, and concentrated under reduced pressured. The residue was purified by silica gel chromatography (0→50% EtOAc/pet. ether) to afford the desired product (88.1 g, 92.6% yield). LCMS (ESI) m/z: [M+H] calcd for C₁₇H₂₅BrN₄O₅S: 477.08; found 477.1.
Step 3. To a solution of 3-(5-bromo-1-ethyl-2-(2-((S)-1-methoxyethyl)pyridin-3-yl)-1H-indol-3-yl)-2,2-dimethylpropan-1-ol (60 g, 134.7 mmol) in toluene (500 mL) at room temperature was added bis(pinacolato)diboron (51.31 g, 202.1 mmol), Pd(dppf)Cl₂ (9.86 g, 13.48 mmol) and KOAc (26.44 g, 269.4 mmol). Then reaction mixture was then heated to 90° C. and stirred for 2 h. The reaction solution was then cooled to room temperature and concentrated under reduced pressure. Purification by silica gel chromatography (0→50% EtOAc/pet. ether) afforded the desired product (60.6 g, 94.0% yield). LCMS (ESI) m/z: [M+H] calcd for C₂₉H₄₁BN₂O₄: 493.32; found 493.3.
Step 4. To a solution of (S)-3-(1-ethyl-2-(2-(1-methoxyethyl)pyridin-3-yl)-5-(4,4,5,5-tetramethyl-1,3,2-dioxaborolan-2-yl)-1H-indol-3-yl)-2,2-dimethylpropan-1-ol (30 g, 60.9 mmol) in toluene (600 mL), dioxane (200 mL), and H₂O (200 mL) at room temperature was added methyl (S)-1-((S)-3-(4-bromothiazol-2-yl)-2-((tert-butoxycarbonyl)amino)propanoyl)hexahydropyridazine-3-carboxylate (43.62 g, 91.4 mmol), K₃PO₄ (32.23 g, 152.3 mmol) and Pd(dppf)Cl₂ (8.91 g, 12.18 mmol). The resulting solution was heated to 70° C. and stirred overnight. The reaction mixture was then cooled to room temperature and was quenched with H₂O (200 mL). The resulting mixture was extracted with EtOAc (3×1000 mL), and the combined organic layers were dried over Na₂SO₄, filtered, and concentrated under reduced pressure. The residue was purified by silica gel chromatography (0→90% EtOAc/pet. ether) to afford the desired product (39.7 g, 85.4% yield). LCMS (ESI) m/z: [M+H] calcd for C₄₀H₅₄N₆O₇S: 763.39; found 763.3.
Step 5. To a solution of methyl (S)-1-((S)-2-((tert-butoxycarbonyl)amino)-3-(4-(1-ethyl-3-(3-hydroxy-2,2-dimethylpropyl)-2-(2-((S)-1-methoxyethyl)pyridin-3-yl)-1H-indol-5-yl)thiazol-2-yl)propanoyl)hexahydropyridazine-3-carboxylate (39.7 g, 52.0 mmol) in THF (400 mL) and H₂O (100 mL) at room temperature was added LiOH·H₂O (3.74 g, 156.2 mmol). The resulting mixture was stirred for 1.5 h and was then concentrated under reduced pressure. The residue was acidified to pH 6 with 1 M HCl and extracted with DCM (3×1000 mL). The combined organic layers were dried over Na₂SO₄, filtered, and concentrated under reduced pressure to afford the desired product (37.9 g, crude). LCMS (ESI) m/z: [M+H] calcd for C₃₉H₅₂N₆O₇S: 749.37; found 749.4.
Step 6. To a solution of (S)-1-((S)-2-((tert-butoxycarbonyl)amino)-3-(4-(1-ethyl-3-(3-hydroxy-2,2-dimethylpropyl)-2-(2-((S)-1-methoxyethyl)pyridin-3-yl)-1H-indol-5-yl)thiazol-2-yl)propanoyl)hexahydropyridazine-3-carboxylic acid (37.9 g, 50.6 mmol), HOBt (34.19 g, 253.0 mmol) and DIPEA (264.4 mL, 1518 mmol) in DCM (4 L) at 0° C. was added EDCl (271.63 g, 1416.9 mmol). The resulting mixture was warmed to room temperature and stirred overnight. The reaction mixture was then quenched with H₂O and washed with 1 M HCl (4×1 L). The organic layer was separated and concentrated under reduced pressure. The residue was purified by silica gel chromatography (0→70% EtOAc/pet. ether) to afford the desired product (30 g, 81.1% yield). LCMS (ESI) m/z: [M+H] calcd for C₃₉H₅₀N₆O₆S: 731.36; found 731.3.

### Intermediate 3. Synthesis of (S)-3-Bromo-5-Iodo-2-(1-Methoxyethyl) Pyridine

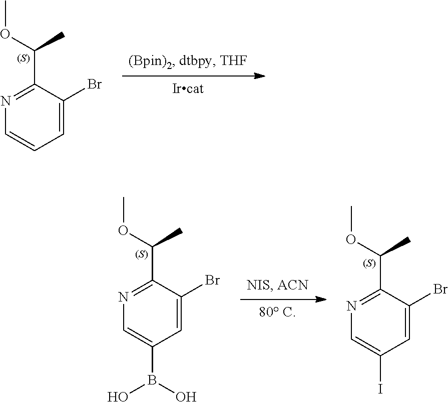

Step 1. To a stirred solution of 3-bromo-2-[(1S)-1-methoxyethyl]pyridine (80.00 g, 370.24 mmol, 1.00 equiv) and bis(pinacolato)diboron (141.03 g, 555.3 mmol, 1.50 equiv) in THF (320 mL) was added dtbpy (14.91 g, 55.5 mmol) and Chloro(1,5-cyclooctadiene)iridium(I) dimer (7.46 g, 11.1 mmol) under argon atmosphere. The resulting mixture was stirred for 16 h at 75° C. under argon atmosphere. The mixture was concentrated under reduced pressure. The resulting mixture was dissolved in EtOAc (200 mL), and the mixture was adjusted to pH 10 with Na₂CO₃ (40 g) and NaOH (10 g) (mass 4:1) in water (600 mL). The aqueous layer was extracted with EtOAc (800 mL). The aqueous phase was acidified to pH=6 with HCl (6 N) to precipitate the desired solid to afford 5-bromo-6-[(1S)-1-methoxyethyl]pyridin-3-ylboronic acid (50 g, 52.0% yield) as a light-yellow solid. LCMS (ESI): m/z [M+H] calc'd for C₈H₁₁BBrNO₃ 259.0; found 260.0.
Step 2. To a stirred solution of 5-bromo-6-[(1S)-1-methoxyethyl]pyridin-3-ylboronic acid (23.00 g, 88.5 mmol) in ACN (230 mL) were added NIS (49.78 g, 221.2 mmol) at room temperature under argon atmosphere. The resulting mixture was stirred for overnight at 80° C. under argon atmosphere. The resulting mixture was concentrated under reduced pressure. The resulting mixture was dissolved in DCM (2.1 L) and washed with Na₂S₂O₃ (3×500 mL). The organic layer was dried over anhydrous Na₂SO₄. After filtration, the filtrate was concentrated under reduced pressure. The residue was purified by silica gel column chromatography to afford (S)-3-bromo-5-iodo-2-(1-methoxyethyl)pyridine (20 g, 66.0% yield). LCMS (ESI): m/z [M+H] calc'd for C₈H₉BrINO 340.9; found 341.7.

### Intermediate 4. Synthesis of tert-butyl ((6³S,4S,Z)-1¹-ethyl-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)carbamate

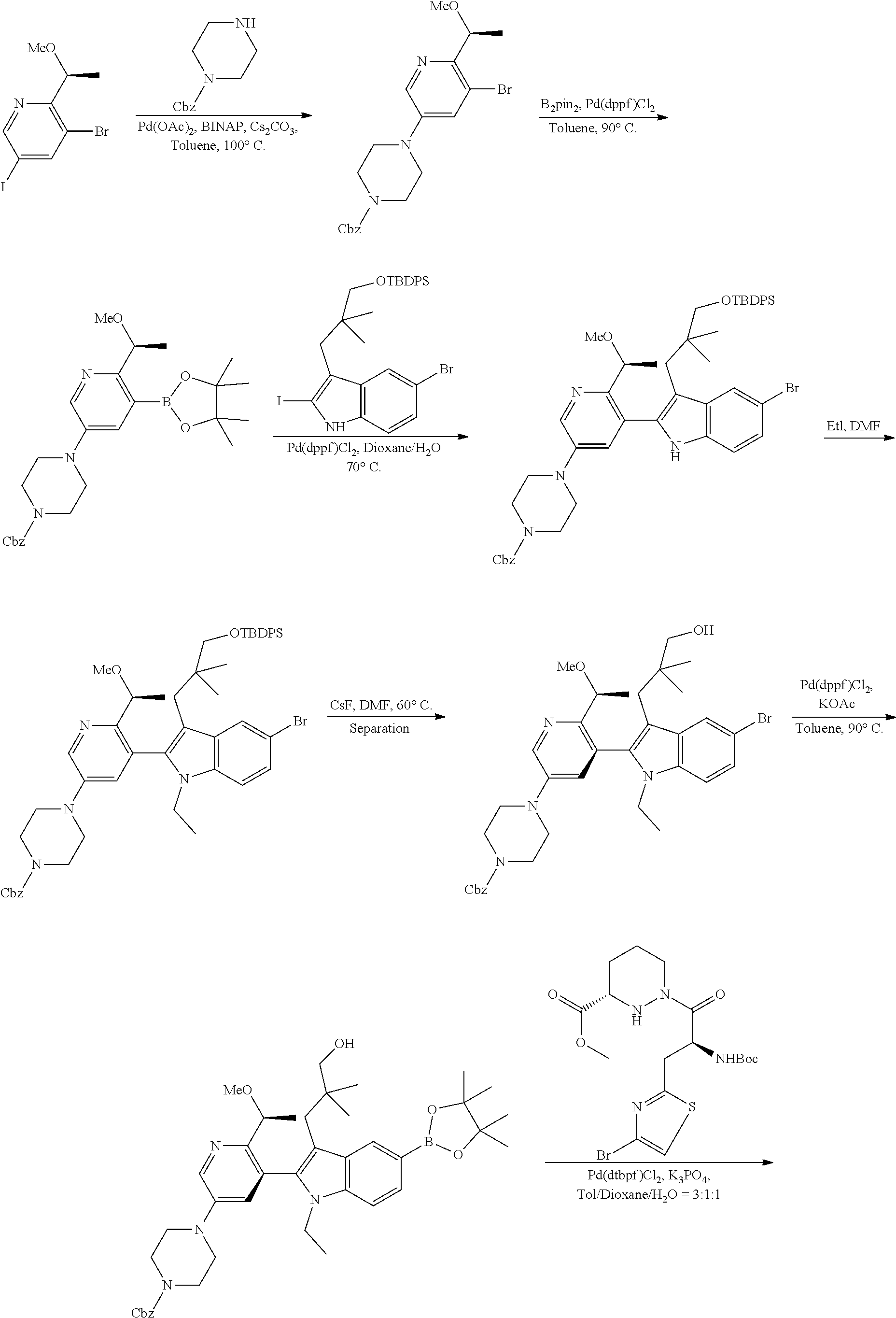

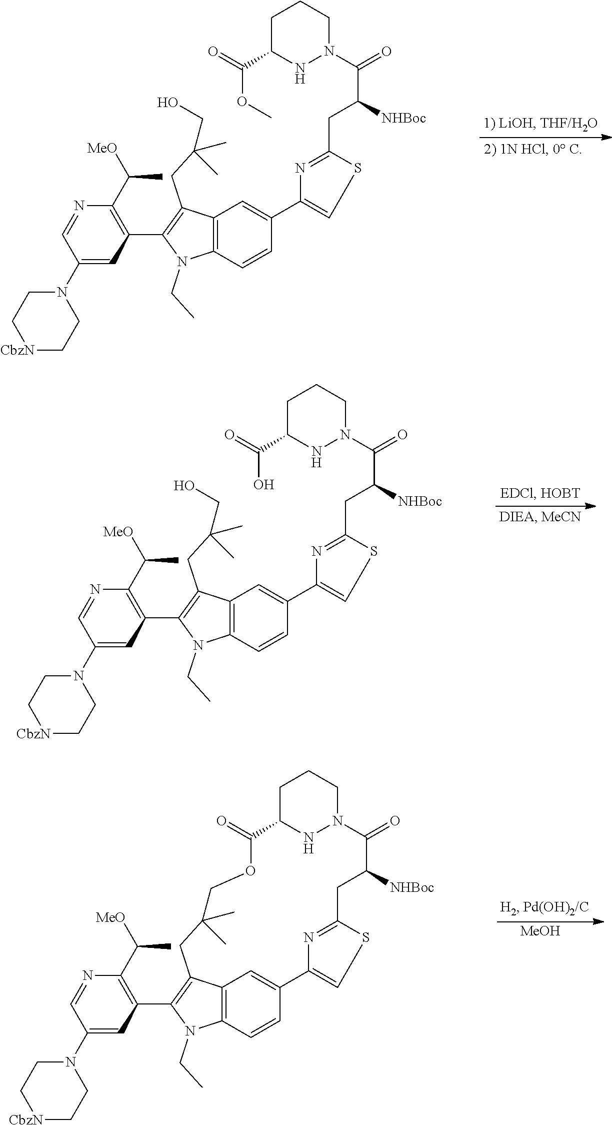

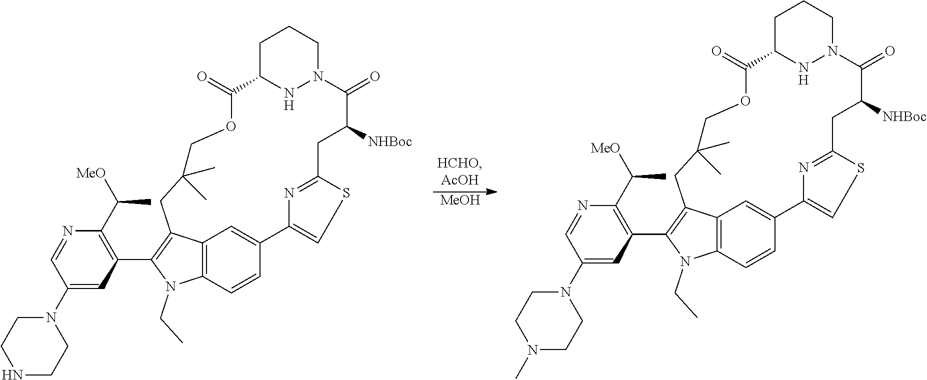

Step 1. Into a 3 L 3-necked round-bottom flask purged and maintained with an inert atmosphere of argon, was placed 3-bromo-5-iodo-2-[(1S)-1-methoxyethyl]pyridine (147 g, 429.8 mmol) benzyl piperazine-1-carboxylate (94.69 g, 429.8 mmol), Pd (OAc) 2 (4.83 g, 21.4 mmol), BINAP (5.35 g, 8.6 mmol), Cs₂CO₃ (350.14 g, 1074.6 mmol), toluene (1 L). The resulting solution was stirred for overnight at 100° C. in an oil bath. The reaction mixture was cooled to 25° C. after reaction completed. The resulting mixture was concentrated under reduced pressure. The residue was applied onto a silica gel column with ethyl acetate/hexane (1:1). Removal of solvent under reduced pressure gave benzyl (S)-4-(5-bromo-6-(1-methoxyethyl)pyridin-3-yl) piperazine-1-carboxylate (135 g, 65.1% yield) as a dark yellow solid. LCMS (ESI): m/z [M+H] calc'd for C₂₀H₂₄BrN₃O₃ 433.1; found 434.1.
Step 2. Into a 3-L 3-necked round-bottom flask purged and maintained with an inert atmosphere of argon, was placed benzyl 4-[5-bromo-6-[(1S)-1-methoxyethyl]pyridin-3-yl]piperazine-1-carboxylate (135 g, 310.8 mmol), bis(pinacolato)diboron (86.82 g, 341.9 mmol), Pd(dppf)Cl₂ (22.74 g, 31.0 mmol), KOAc (76.26 g, 777.5 mmol), Toluene (1 L). The resulting solution was stirred for 2 days at 90° C. in an oil bath. The reaction mixture was cooled to 25° C. The resulting mixture was concentrated under vacuum. The residue was applied onto a neutral alumina column with ethyl acetate/hexane (1:3). Removal of solvent under reduced pressure gave benzyl (S)-4-(6-(1-methoxyethyl)-5-(4,4,5,5-tetramethyl-1,3,2-dioxaborolan-2-yl)pyridin-3-yl) piperazine-1-carboxylate (167 g, crude) as a dark yellow solid. LCMS (ESI): m/z [M+H] calc'd for C₂₆H₃₆BN₃O₅ 481.3; found 482.1.
Step 3. Into a 3-L 3-necked round-bottom flask purged and maintained with an inert atmosphere of argon, was placed(S)-4-(6-(1-methoxyethyl)-5-(4,4,5,5-tetramethyl-1,3,2-dioxaborolan-2-yl)pyridin-3-yl) piperazine-1-carboxylate (167 g, 346.9 mmol), 5-bromo-3-[3-[(tert-butyldiphenylsilyl)oxy]-2,2-dimethylpropyl]-2-iodo-1H-indole (224.27 g, 346.9 mmol), Pd(dppf)Cl₂ (25.38 g, 34.6 mmol), dioxane (600 mL), H₂O (200 mL), K₃PO₄ (184.09 g, 867.2 mmol), Toluene (200 mL). The resulting solution was stirred for overnight at 70° C. in an oil bath. The reaction mixture was cooled to 25° C. after reaction completed. The resulting mixture was concentrated under vacuum. The residue was applied onto a silica gel column with ethyl acetate/hexane (1:1). Removal of solvent under reduced pressure gave benzyl (S)-4-(5-(5-bromo-3-(3-((tert-butyldiphenylsilyl)oxy)-2,2-dimethylpropyl)-1H-indol-2-yl)-6-(1-methoxyethyl)pyridin-3-yl) piperazine-1-carboxylate (146 g, 48.1% yield) as a yellow solid. LCMS (ESI): m/z [M+H] calc'd for C₄₉H₅₇BrN₄O₄Si 872.3; found 873.3.
Step 4. To a stirred mixture of benzyl (S)-4-(5-(5-bromo-3-(3-((tert-butyldiphenylsilyl)oxy)-2,2-dimethylpropyl)-1H-indol-2-yl)-6-(1-methoxyethyl)pyridin-3-yl) piperazine-1-carboxylate (146 g, 167.0 mmol) and Cs₂CO₃ (163.28 g, 501.1 mmol) in DMF (1200 mL) was added C₂H₅I (52.11 g, 334.0 mmol) in portions at 0° C. under N₂ atmosphere. The final reaction mixture was stirred at 25° C. for 12 h. Desired product could be detected by LCMS. The resulting mixture was diluted with EA (1 L) and washed with brine (3×1.5 L). The organic layers were dried over anhydrous Na₂SO₄. After filtration, the filtrate was concentrated under reduced pressure to give benzyl (S)-4-(5-(5-bromo-3-(3-((tert-butyldiphenylsilyl)oxy)-2,2-dimethylpropyl)-1-ethyl-1H-indol-2-yl)-6-(1-methoxyethyl)pyridin-3-yl) piperazine-1-carboxylate (143 g, crude) as a yellow solid that was used directly for next step without further purification. LCMS (ESI): m/z [M+H] calc'd for C₅₁H₆₁BrN₄O₄Si 900.4; found 901.4.
Step 5. To a stirred mixture of benzyl benzyl (S)-4-(5-(5-bromo-3-(3-((tert-butyldiphenylsilyl)oxy)-2,2-dimethylpropyl)-1-ethyl-1H-indol-2-yl)-6-(1-methoxyethyl)pyridin-3-yl) piperazine-1-carboxylate (143 g, 158.5 mmol) in DMF (1250 mL) was added CsF (72.24 g, 475.5 mmol). Then the reaction mixture was stirred at 60° C. for 2 days under N₂ atmosphere. Desired product could be detected by LCMS. The resulting mixture was diluted with EA (1 L) and washed with brine (3×1 L). Then the organic phase was concentrated under reduced pressure. The residue was purified by silica gel column chromatography, eluted with PE/EA (1/3) to afford two atropisomers of benzyl (S)-4-(5-(5-bromo-1-ethyl-3-(3-hydroxy-2,2-dimethylpropyl)-1H-indol-2-yl)-6-(1-methoxyethyl)pyridin-3-yl) piperazine-1-carboxylate A (38 g, 36% yield, RT=1.677 min in 3 min LCMS (0.1% FA)) and B (34 g, 34% yield, RT=1.578 min in 3 min LCMS (0.1% FA)) both as yellow solid. LCMS (ESI): m/z [M+H] calc'd for C₃₅H₄₃BrN₄O₄ 663.2; found 662.2.
Step 6. Into a 500-mL 3-necked round-bottom flask purged and maintained with an inert atmosphere of nitrogen, was placed benzyl (S)-4-(5-(5-bromo-1-ethyl-3-(3-hydroxy-2,2-dimethylpropyl)-1H-indol-2-yl)-6-(1-methoxyethyl)pyridin-3-yl) piperazine-1-carboxylate A (14 g, 21.1 mmol), bis(pinacolato)diboron (5.89 g, 23.21 mmol), Pd(dppf)Cl₂ (1.54 g, 2.1 mmol), KOAc (5.18 g, 52.7 mmol), Toluene (150 mL). The resulting solution was stirred for 5 h at 90° C. in an oil bath. The reaction mixture was cooled to 25° C. The resulting mixture was concentrated under vacuum. The residue was purified by silica gel column chromatography, eluted with PE/EA (1/3) to give benzyl (S)-4-(5-(1-ethyl-3-(3-hydroxy-2,2-dimethylpropyl)-5-(4,4,5,5-tetramethyl-1,3,2-dioxaborolan-2-yl)-1H-indol-2-yl)-6-(1-methoxyethyl)pyridin-3-yl) piperazine-1-carboxylate (12 g, 76.0% yield) as a yellow solid. LCMS (ESI): m/z [M+H] calc'd for C₄₁H₅₅BN₄O₆ 710.4; found 711.3.
Step 7. Into a 250-mL round-bottom flask purged and maintained with an inert atmosphere of argon, was placed benzyl (S)-4-(5-(1-ethyl-3-(3-hydroxy-2,2-dimethylpropyl)-5-(4,4,5,5-tetramethyl-1,3,2-dioxaborolan-2-yl)-1H-indol-2-yl)-6-(1-methoxyethyl)pyridin-3-yl) piperazine-1-carboxylate (10.8 g, 15.2 mmol), methyl (3S)-1-[(2S)-3-(4-bromo-1,3-thiazol-2-yl)-2-[(tert-butoxycarbonyl)amino]propanoyl]-1,2-diazinane-3-carboxylate (7.98 g, 16.7 mmol), Pd(dtbpf)Cl₂ (0.99 g, 1.52 mmol), K₃PO₄ (8.06 g, 37.9 mmol), Toluene (60 mL), dioxane (20 mL), H₂O (20 mL). The resulting solution was stirred for 3 h at 70° C. in an oil bath. The reaction mixture was cooled to 25° C. The resulting solution was extracted with EtOAc (2×50 mL) and concentrated under reduced pressure. The residue was applied onto a silica gel column with ethyl acetate/hexane (10:1). Removal of solvent to give methyl (S)-1-((S)-3-(4-(2-(5-(4-((benzyloxy)carbonyl)piperazin-1-yl)-2-((S)-1-methoxyethyl)pyridin-3-yl)-1-ethyl-3-(3-hydroxy-2,2-dimethylpropyl)-1H-indol-5-yl)thiazol-2-yl)-2-((tert-butoxycarbonyl)amino)propanoyl)hexahydropyridazine-3-carboxylate (8 g, 50.9% yield) as a yellow solid. LCMS (ESI): m/z [M+H] calc'd for C₅₂H₆₈N₈O₉S 980.5; found 980.9.
Step 8. To a stirred mixture of methyl (S)-1-((S)-3-(4-(2-(5-(4-((benzyloxy)carbonyl)piperazin-1-yl)-2-((S)-1-methoxyethyl)pyridin-3-yl)-1-ethyl-3-(3-hydroxy-2,2-dimethylpropyl)-1H-indol-5-yl)thiazol-2-yl)-2-((tert-butoxycarbonyl)amino)propanoyl)hexahydropyridazine-3-carboxylate (12 g, 12.23 mmol) in THF (100 mL)/H₂O (100 mL) was added LiOH (2.45 g, 61.1 mmol) under N₂ atmosphere and the resulting mixture was stirred for 2 h at 25° C. Desired product could be detected by LCMS. THF was concentrated under reduced pressure. The pH of aqueous phase was acidified to 5 with HCL (1N) at 0° C. The aqueous layer was extracted with DCM (3×100 ml). The organic phase was concentrated under reduced pressure to give (S)-1-((S)-3-(4-(2-(5-(4-((benzyloxy)carbonyl)piperazin-1-yl)-2-((S)-1-methoxyethyl)pyridin-3-yl)-1-ethyl-3-(3-hydroxy-2,2-dimethylpropyl)-1H-indol-5-yl)thiazol-2-yl)-2-((tert-butoxycarbonyl)amino)propanoyl)hexahydropyridazine-3-carboxylic acid (10 g, 84.5% yield) as a light yellow solid. LCMS (ESI): m/z [M+H] calc'd for C₅₁H₆₆N₈O₉S 966.5; found 967.0.
Step 9. Into a 3-L round-bottom flask purged and maintained with an inert atmosphere of nitrogen, was placed(S)-1-((S)-3-(4-(2-(5-(4-((benzyloxy)carbonyl)piperazin-1-yl)-2-((S)-1-methoxyethyl)pyridin-3-yl)-1-ethyl-3-(3-hydroxy-2,2-dimethylpropyl)-1H-indol-5-yl)thiazol-2-yl)-2-((tert-butoxycarbonyl)amino)propanoyl)hexahydropyridazine-3-carboxylic acid (18 g, 18.61 mmol), ACN (1.8 L), DIEA (96.21 g, 744.4 mmol), EDCl (107.03 g, 558.3 mmol), HOBT (25.15 g, 186.1 mmol). The resulting solution was stirred for overnight at 25° C. The resulting mixture was concentrated under vacuum after reaction completed. The resulting solution was diluted with DCM (1 L). The resulting mixture was washed with HCl (3×1 L, 1N aqueous). The resulting mixture was washed with water (3×1 L). Then the organic layer was concentrated, the residue was applied onto a silica gel column with ethyl acetate/hexane (1:1). Removal of solvent under reduced pressure gave benzyl 4-(5-((6³S,4S,Z)-4-((tert-butoxycarbonyl)amino)-1¹-ethyl-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)- pyridazinacycloundecaphane-1²-yl)-6-((S)-1-methoxyethyl)pyridin-3-yl) piperazine-1-carboxylate (10.4 g, 54.8% yiels) as a light yellow solid. LCMS (ESI): m/z [M+H] calc'd for C₅₁H₆₄N₈O₈S 948.5; found 949.3.
Step 10. Into a 250-mL round-bottom flask purged and maintained with an inert atmosphere of nitrogen, was placed benzyl 4-(5-((6³S,4S,Z)-4-((tert-butoxycarbonyl)amino)-1¹-ethyl-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-1²-yl)-6-((S)-1-methoxyethyl)pyridin-3-yl) piperazine-1-carboxylate (10.40 g, 10.9 mmol), Pd(OH)₂/C (5 g, 46.9 mmol), MeOH (100 mL). The resulting solution was stirred for 3 h at 25° C. under 2 atm H₂ atmosphere. The solids were filtered out and the filter cake was washed with MeOH (3×100 mL). Then combined organic phase was concentrated under reduced pressure to give tert-butyl ((6³S,4S,Z)-11-ethyl-1²-(2-((S)-1-methoxyethyl)-5-(piperazin-1-yl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)carbamate (8.5 g, 90.4% yield) as a light yellow solid. LCMS (ESI): m/z [M+H] calc'd for C₄₃H₅₈N₈O₆S 814.4; found 815.3.
Step 11. Into a 1000-mL round-bottom flask purged and maintained with an inert atmosphere of nitrogen, was placed tert-butyl ((6³S,4S,Z)-11-ethyl-1²-(2-((S)-1-methoxyethyl)-5-(piperazin-1-yl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)- pyridazinacycloundecaphane-4-yl)carbamate (8.5 g, 10.4 mmol), MeOH (100 mL), AcOH (1.88 g, 31.2 mmol) and stirred for 15 mins. Then HCHO (1.88 g, 23.15 mmol, 37% aqueous solution) and NaBH₃CN (788 mg, 12.5 mmol) was added at 25° C. The resulting solution was stirred for 3 h at 25° C. The resulting mixture was quenched with 100 ml water and concentrated under reduced pressure to remove MeOH. The resulting solution was diluted with 300 mL of DCM. The resulting mixture was washed with water (3×100 mL). Removal of solvent gave tert-butyl ((6³S,4S,Z)-1¹-ethyl-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)carbamate (8.2 g, 90.1% yield) as a yellow solid. LCMS (ESI): m/z [M+H] calc'd for C₄₄H₆₀N₈O₆S 828.4; found 829.3.

### Intermediate 5. Synthesis of (6³S,4S)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

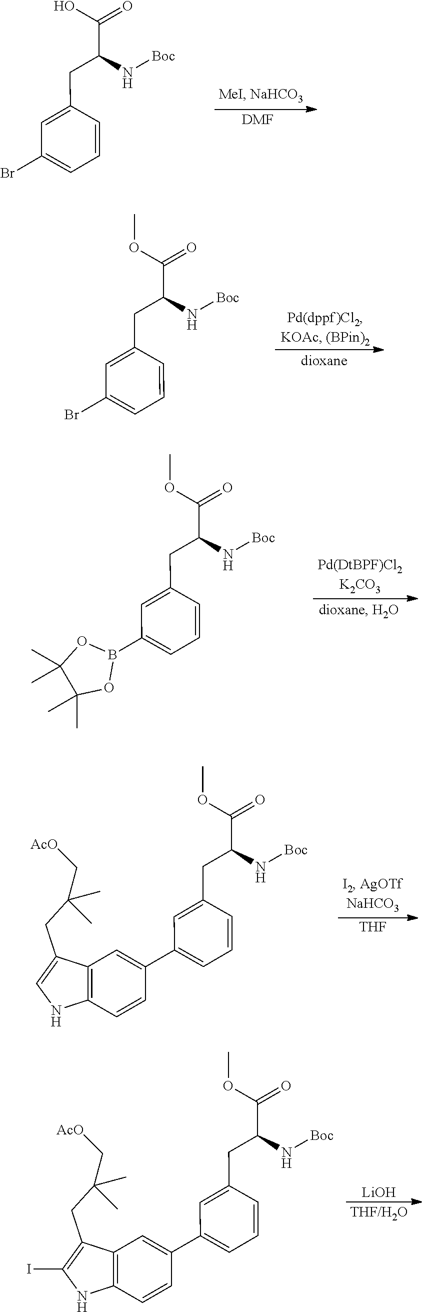

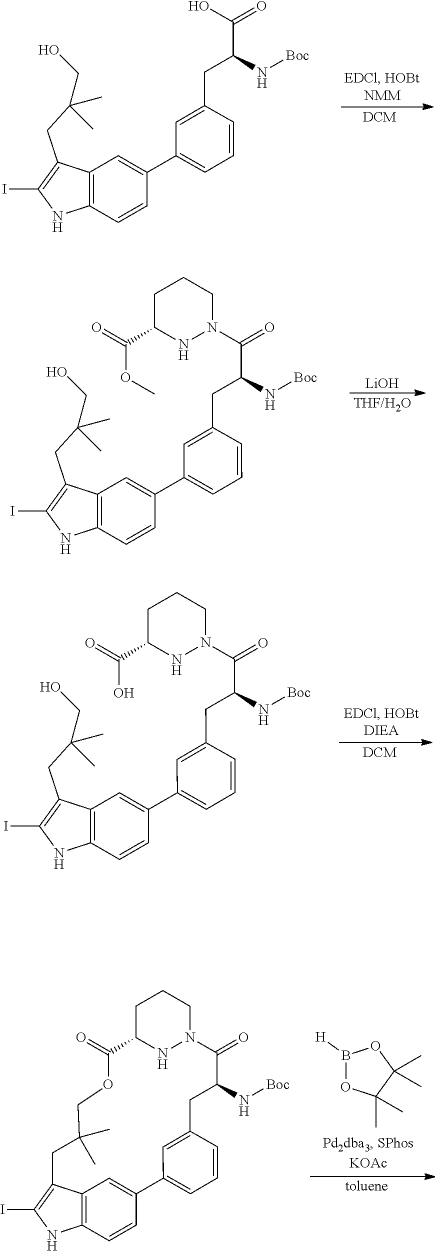

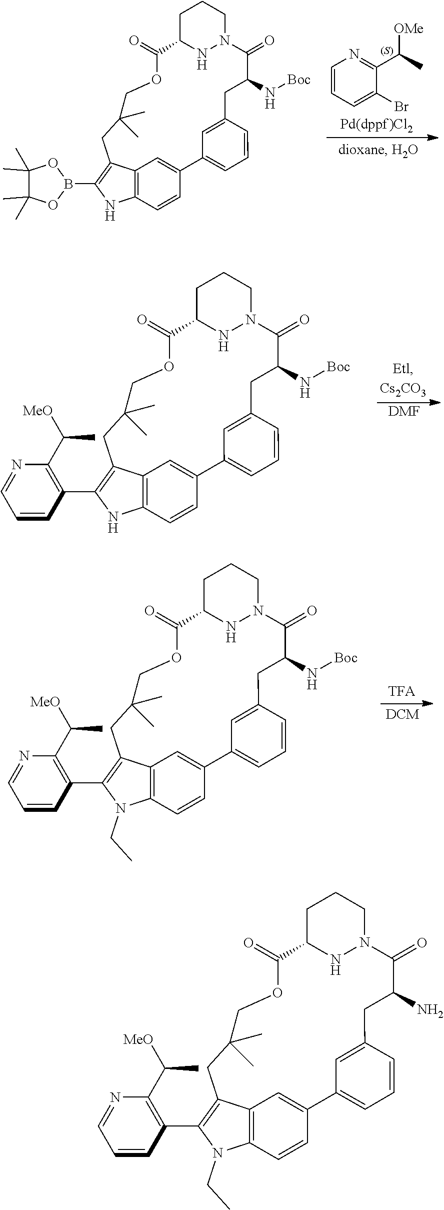

Step 1. To a solution of (2S)-3-(3-bromophenyl)-2-[(tert-butoxycarbonyl)amino]propanoic acid (100 g, 290 mmol) in DMF (1 L) at room temperature was added NaHCO₃ (48.8 g, 581.1 mmol) and Mel (61.9 g, 435.8 mmol). The reaction mixture was stirred for 16 h and was then quenched with H₂O (1 L) and extracted with EtOAc (3×1 L). The combined organic layers were washed with brine (3×500 mL), dried over Na₂SO₄, filtered, and concentrated under reduced pressure. The residue was purified by silica gel column chromatography (13% EtOAc/pet. ether) to give the final product (109 g, crude). LCMS (ESI) m/z [M+Na] calcd for C₁₅H₂₀BrNO₄ 380.05; found: 380.0.
Step 2. To a stirred solution of methyl (2S)-3-(3-bromophenyl)-2-[(tert-butoxycarbonyl)amino]propanoate (108 g, 301.5 mmol) and bis(pinacolato)diboron (99.53 g, 391.93 mmol) in dioxane (3.2 L) was added KOAc (73.97 g, 753.70 mmol) and Pd(dppf)Cl₂ (22.06 g, 30.15 mmol). The reaction mixture was heated to 90° C. for 3 h and was then cooled to room temperature and extracted with EtOAc (2×3 L). The combined organic layers were washed with brine (3×800 mL), dried over Na₂SO₄, filtered, and concentrated under reduced pressure. The residue was purified by silica gel column chromatography (5% EtOAc/pet. ether) to afford the product (96 g, 78.6% yield). LCMS (ESI) m/z [M+Na] calcd for C₂₁H₃₂BNO₆ 428.22; found: 428.1.
Step 3. To a mixture of methyl (2S)-2-[(tert-butoxycarbonyl)amino]-3-[3-(4,4,5,5-tetramethyl-1,3,2-dioxaborolan-2-yl)phenyl]propanoate (94 g, 231.9 mmol) and 3-(5-bromo-1H-indol-3-yl)-2,2-dimethylpropyl acetate (75.19 g, 231.93 mmol) in dioxane (1.5 L) and H₂O (300 mL) was added K₂CO₃ (64.11 g, 463.85 mmol) and Pd(DtBPF)Cl₂ (15.12 g, 23.19 mmol). The reaction mixture was heated to 70° C. and stirred for 4 h. The reaction mixture was extracted with EtOAc (2×2 L), and the combined organic layers were washed with brine (3×600 mL), dried over Na₂SO₄, filtered, and concentrated under reduced pressure. The residue was purified by silica gel column chromatography (20% EtOAc/pet. ether) to give the product (130 g, crude). LCMS (ESI) m/z [M+H] calcd for C₃₀H₃₈N₂O₆ 523.28; found: 523.1.
Step 4. To a solution of methyl (2S)-3-(3-[3-[3-(acetyloxy)-2,2-dimethylpropyl]-1H-indol-5-yl]phenyl)-2-[(tert-butoxycarbonyl)amino]propanoate (95.0 g, 181.8 mmol) and iodine (36.91 g, 145.41 mmol) in THF (1 L) at −10° C. was added AgOTf (70.0 g, 272.7 mmol) and NaHCO₃ (22.9 g, 272.65 mmol). The reaction mixture was stirred for 30 min and was then quenched by the addition of sat. aq. Na₂S₂O₃ (100 mL) at 0° C. The resulting mixture was extracted with EtOAc (3×1 L), and the combined organic layers were washed with brine (3×500 mL), dried over Na₂SO₄, filtered, and concentrated under reduced pressure. The residue was purified by silica gel column chromatography (50% EtOAc/pet. ether) to give methyl (S)-3-(3-(3-(3-acetoxy-2,2-dimethylpropyl)-2-iodo-1H-indol-5-yl)phenyl)-2-((tert-butoxycarbonyl)amino)propanoate (49.3 g, 41.8% yield). LCMS (ESI) m/z [M+H] calcd for C₃₀H₃₇IN₂O₆: 649.18; found: 649.1.
Step 5. To a solution of methyl (2S)-3-(3-[3-[3-(acetyloxy)-2,2-dimethylpropyl]-2-iodo-1H-indol-5-yl]phenyl)-2-[(tert-butoxycarbonyl)amino]propanoate (60 g, 92.5 mmol) in THF (600 mL) was added a solution of LiOH·H₂O (19.41 g, 462.5 mmol) in H₂O (460 mL). The resulting solution was stirred overnight and then the pH was adjusted to 6 with HCl (1 M). The resulting solution was extracted with EtOAc (2×500 mL), and the combined organic layers was washed with brine (2×500 mL), dried over Na₂SO₄, filtered, and concentrated under reduced pressure to give the product (45 g, 82.1% yield). LCMS (ESI) m/z [M+Na] calcd for C₂₇H₃₃IN₂O₆ 615.13; found: 615.1.
Step 6. To a solution of (2S)-2-[(tert-butoxycarbonyl)amino]-3-[3-[3-(3-hydroxy-2,2-dimethylpropyl)-2-iodo-1H-indol-5-yl]phenyl]propanoic acid (30 g, 50.6 mmol) and methyl (3S)-1,2-diazinane-3-carboxylate (10.9 g, 75.9 mmol) in DCM (400 mL) was added NMM (40.97 g, 405.08 mmol), HOBt (2.05 g, 15.19 mmol), and EDCl (19.41 g, 101.27 mmol). The reaction mixture was stirred overnight and then the mixture was washed with sat. aq. NH₄Cl (2×200 mL) and brine (2×200 mL), and the mixture was dried over Na₂SO₄, filtered, and concentrated under reduced pressure to give the product (14 g, 38.5% yield). LCMS (ESI) m/z [M+H] calcd for C₃₃H₄₃IN₄O₆ 718.23; found: 719.4.
Step 7. To a solution of methyl (S)-1-((S)-2-((tert-butoxycarbonyl)amino)-3-(3-(3-(3-hydroxy-2,2-dimethylpropyl)-2-iodo-1H-indol-5-yl)phenyl)propanoyl)hexahydropyridazine-3-carboxylate (92 g, 128.0 mmol) in THF (920 mL) at 0° C. was added a solution of LiOH·H₂O (26.86 g, 640.10 mmol) in H₂O (640 mL). The reaction mixture was stirred for 2 h and was then concentrated under reduced pressure to give the product (90 g, crude). LCMS (ESI) m/z [M+H] calcd for C₃₂H₄₁IN₄O₆ 705.22; found: 705.1.
Step 8. To a solution of (3S)-1-[(2S)-2-[(tert-butoxycarbonyl)amino]-3-[3-[3-(3-hydroxy-2,2-dimethylpropyl)-2-iodo-1H-indol-5-yl]phenyl]propanoyl]-1,2-diazinane-3-carboxylic acid (90 g, 127.73 mmol) in DCM (10 L) at 0° C. was added HOBt (34.52 g, 255.46 mmol), DIPEA (330.17 g, 2554.62 mmol) and EDCl (367.29 g, 1915.96 mmol). The reaction mixture was stirred for 16 h and was then concentrated under reduced pressure. The mixture was extracted with DCM (2×2 L) and the combined organic layers were washed with brine (3×1 L), dried over Na₂SO₄, filtered, and concentrated under reduced pressure. The residue was purified by silica gel column chromatography (50% EtOAc/pet. ether) to give the product (70 g, 79.8% yield). LCMS (ESI) m/z [M+H] calcd for C₃₂H₃₉IN₄O₅ 687.21; found: 687.1.
Step 9. A 1 L round-bottom flask was charged with tert-butyl ((6³S,4S)-1²-iodo-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (22.0 g, 32.042 mmol), toluene (300.0 mL), Pd₂(dba)₃ (3.52 g, 3.845 mmol), S-Phos (3.95 g, 9.613 mmol), and KOAc (9.43 g, 96.127 mmol) at room temperature. To the mixture was added 4,4,5,5-tetramethyl-1,3,2-dioxaborolane (26.66 g, 208.275 mmol) dropwise with stirring at room temperature. The resulting solution was stirred for 3 h at 60° C. The resulting mixture was filtered, and the filter cake was washed with EtOAc. The filtrate was concentrated under reduced pressure and the remaining residue was purified by silica gel column chromatography to afford the product (22 g, 90% yield) as a solid. LCMS (ESI) m/z [M+H] calcd for C₃₈H₅₁ BN₄O₇ 687.3; found: 687.4.
Step 10. A mixture of tert-butyl ((6³S,4S)-10,10-dimethyl-5,7-dioxo-1²-(4,4,5,5-tetramethyl-1,3,2-dioxaborolan-2-yl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (2.0 g, 2.8 mmol), 3-bromo-2-[(1S)-1-methoxyethyl]pyridine (0.60 g, 2.8 mmol), Pd(dppf)Cl₂ (0.39 g, 0.5 mmol), and K₃PO₄ (1.2 g, 6.0 mmol) in dioxane (50 mL) and H₂O (10 mL) under an atmosphere of N₂ was heated to 70° C. and stirred for 2 h. The mixture was diluted with H₂O (50 mL) and extracted with EtOAc (3×50 mL). The combined organic layers were washed with brine (3×50 mL), dried over anhydrous Na₂SO₄, and filtered. The filtrate was concentrated under reduced pressure and the residue was purified by silica gel column chromatography to afford the product (1.5 g, 74% yield) as a solid. LCMS (ESI) m/z [M+H] calcd for C₄₀H₄₉N₅O₆ 695.4; found: 696.5.
Step 11. To a solution of tert-butyl ((6³S,4S)-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (20 g, 28.7 mmol) and Cs₂CO₃ (18.7 g, 57.5 mmol) in DMF (150 mL) at 0° C. was added a solution of Etl (13.45 g, 86.22 mmol) in DMF (50 mL). The resulting mixture was stirred overnight at 35° C. and then diluted with H₂O (500 mL). The mixture was extracted with EtOAc (2×300 mL), and the combined organic layers were washed with brine (3×100 mL), dried over Na₂SO₄, filtered, and concentrated under reduced pressure. The residue was purified by silica gel column chromatography to afford the product (4.23 g, 18.8% yield) and the atropisomer (5.78 g, 25.7% yield) as solids. LCMS (ESI) m/z [M+H] calcd for C₄₂H₅₃N₅O₆ 724.4; found: 724.6.
Step 12. A mixture of tert-butyl ((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (1.3 g, 1.7 mmol) in TFA (10 mL) and DCM (20 mL) was stirred at 0° C. for 2 h. The mixture was concentrated under reduced pressure to afford the product (1.30 g, crude) as a solid. LCMS (ESI) m/z [M+H] calcd for C₃₇H₄₅N₅O₄ 623.3; found: 624.4.

### Intermediate 6: Synthesis of (S)-3-(5-bromo-1-ethyl-2-(5-iodo-2-(1-methoxyethyl)pyridin-3-yl)-1H-indol-3-yl)-2,2-dimethylpropyl acetate

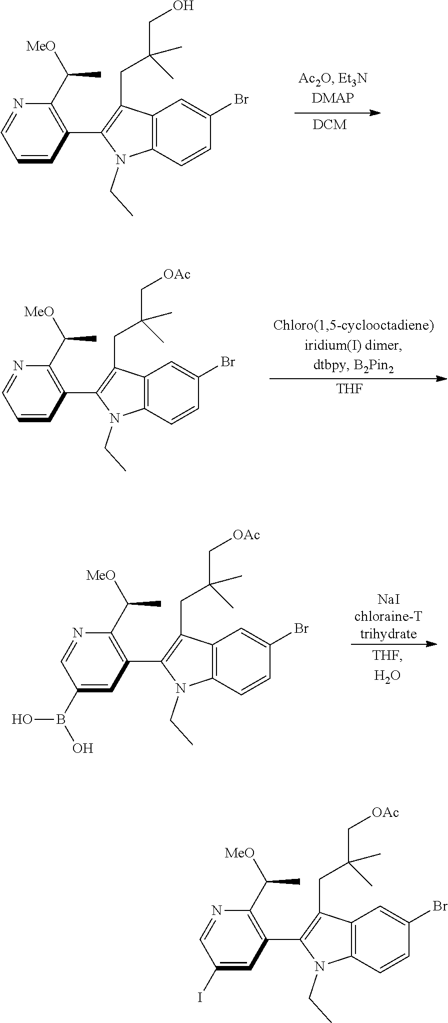

Step 1. To a stirred solution of (S)-3-(5-bromo-1-ethyl-2-(2-(1-methoxyethyl)pyridin-3-yl)-1H-indol-3-yl)-2,2-dimethylpropan-1-ol (100 g, 224.517 mmol) and Et₃N (45.44 g, 449.034 mmol) in DCM (1 L) was added DMAP (2.74 g, 22.452 mmol) and Ac₂O (27.50 g, 269.420 mmol) in portions at 0° C. under an argon atmosphere. The resulting mixture was stirred for 3 h at room temperature. The resulting mixture was concentrated under reduced pressure then diluted with EtOAc (1000 mL). The resulting mixture was washed with 1M HCl (500 mL) then washed with sat. NaHCO₃ (500 mL) and brine (500 mL) dried over anhydrous Na₂SO₄. After filtration, the filtrate was concentrated under reduced pressure. The residue was purified by trituration with pet. ether (500 mL) to afford the product (93.3 g, 85% yield) as a white solid. LCMS (ESI) m/z [M+H] calcd for C₂₅H₃₁BrN₂O₃: 487.16; found: 489.2
Step 2. To a stirred solution of (S)-3-(5-bromo-1-ethyl-2-(2-(1-methoxyethyl)pyridin-3-yl)-1H-indol-3-yl)-2,2-dimethylpropyl acetate (93.3 g, 191.409 mmol) and B₂PIN₂ (72.91 g, 287.113 mmol) in THF (370 mL) was added dtbpy (7.71 g, 28.711 mmol) and chloro (1,5-cyclooctadiene) iridium (I) dimer (6.43 g, 9.570 mmol) in portions at room temperature under an argon atmosphere. The resulting mixture was stirred overnight at 75° C. The resulting mixture was concentrated under reduced pressure to afford the product (190 g, crude) as an oil. LCMS(ESI) m/z [M+H]; calcd for C₂₅H₃₂BBrN₂O₅: 531.17; found: 533.3
Step 3. To a stirred solution of (S)-(5-(3-(3-acetoxy-2,2-dimethylpropyl)-5-bromo-1-ethyl-1H-indol-2-yl)-6-(1-methoxyethyl)pyridin-3-yl)boronic acid (110 g, 207.059 mmol) and chloramine-T trihydrate (349.96 g, 1242.354 mmol) in THF (550 mL) was added a solution of NaI (186.22 g, 1242.354 mmol) in H₂O (225 mL) in portions at 0° C. under an air atmosphere. The resulting mixture was stirred overnight at 50° C. under an argon atmosphere. The resulting mixture was concentrated under reduced pressure then washed with CHCl₃ (500 mL). The resulting mixture was filtered, the filter cake was washed with CHCl₃ (3×250 mL). The filtrate was extracted with CHCl₃ (3×500 mL). The combined organic layers were washed with Na₂S₂O₃ (500 mL), washed with brine (2×200 mL) dried over anhydrous Na₂SO₄. After filtration, the filtrate was concentrated under reduced pressure. The residue was purified by silica gel column

### Intermediate 7: Synthesis of 3-(5-bromo-1-ethyl-2-(2-((S)-1-methoxyethyl)-5-((R)-octahydro-2H-pyrido[1,2-a]pyrazin-2-yl)pyridin-3-yl)-1H-indol-3-yl)-2,2-dimethylpropyl acetate

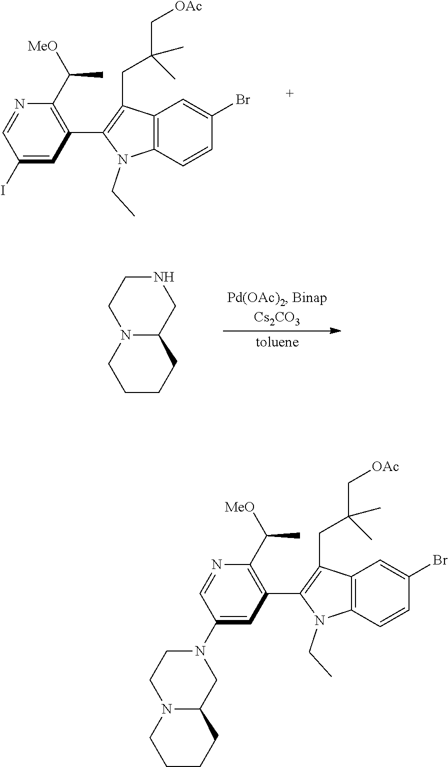

Step 1. To a stirred solution of 3-(5-bromo-1-ethyl-2-{5-iodo-2-[(1S)-1-methoxyethyl]pyridin-3-yl}indol-3-yl)-2,2-dimethylpropyl acetate (9 g, 14.674 mmol), (R)-octahydro-2H-pyrido[1,2-a]pyrazine (2.469 g, 17.609 mmol), Cs₂CO₃ (11.9523 g, 36.685 mmol) and BINAP (456.85 mg, 0.734 mmol) in toluene (63 mL) was added Pd (OAc) 2 (329.44 mg, 1.467 mmol) in portions at room temperature under an argon atmosphere. The resulting mixture was stirred for 6 h at 100° C. then the mixture was filtered, the filter cake was washed with EtOAc (100 mL). The filtrate was concentrated under reduced pressure. The residue was purified by prep-TLC (8% MeOH/DCM) to afford the product (6 g, 65% yield) as a solid. LCMS (ESI) m/z [M+H] calcd C₃₃H₄₅BrN₄O₃: 625.28; found: 627.4.

### Intermediate 8. Synthesis of (6³S,4S)-4-amino-1¹-ethyl-2⁵-(fluoromethyl)-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

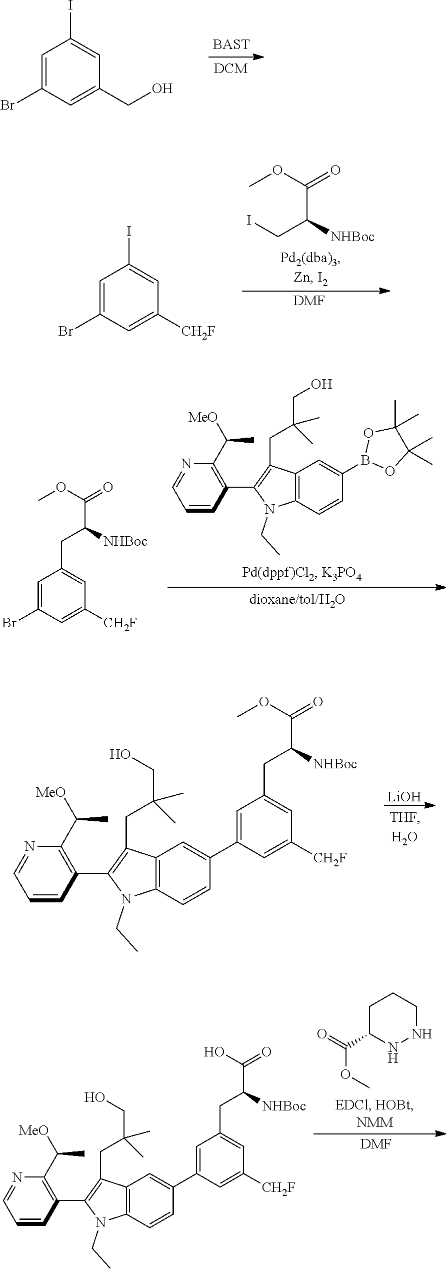

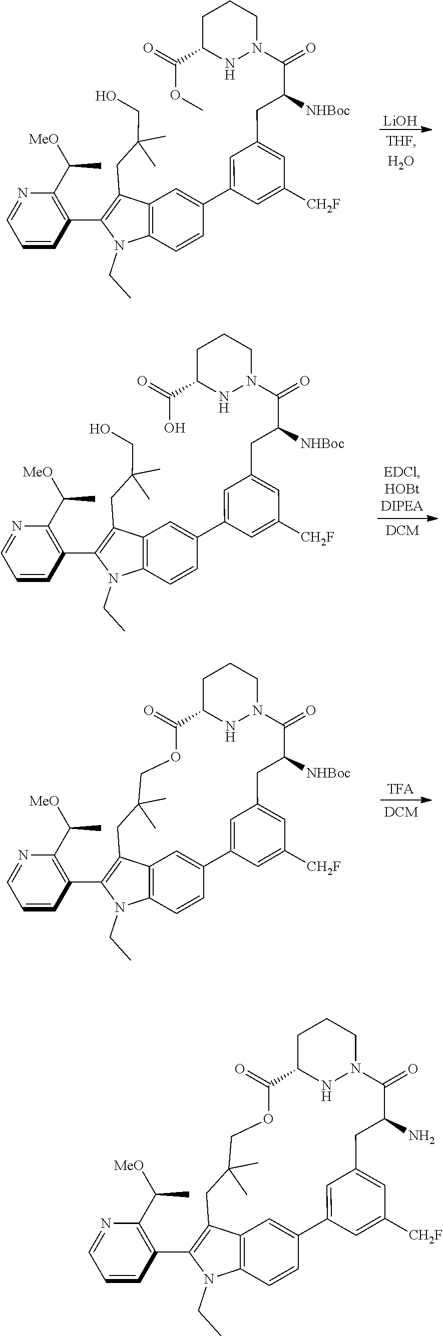

Step 1. To a solution of (3-bromo-5-iodophenyl) methanol (175.0 g, 559.227 mmol) in DCM (2 L) was added BAST (247.45 g, 1118.454 mmol) dropwise at 0° C. The resulting mixture was stirred for 16 h at room temperature. The reaction was quenched with sat. aq. NaHCO₃ at 0° C. The organic layers were washed with H₂O (3×700 mL) and dried over anhydrous Na₂SO₄. After filtration, the filtrate was concentrated under reduced pressure. The residue was purified by silica gel column chromatography (3% EtOAc/pet. ether) to afford the desired product (120 g, 68% yield).
Step 2. Into a 1000 mL 3-necked round-bottom flask was added Zn powder (32.40 g, 495.358 mmol) in DMF (350.0 mL) and 12 (967.12 mg, 3.810 mmol). To the mixture was added a solution of methyl (2R)-2-[(tert-butoxycarbonyl)amino]-3-iodopropanoate (27.0 g, 82.03 mmol) in DMF (10 mL). The mixture was heated to 30° C. for 10 min. To the mixture was then added a solution of methyl (2R)-2-[(tert-butoxycarbonyl)amino]-3-iodopropanoate (54.0 g, 164.07 mmol) in DMF (20 mL). The resulting mixture was stirred for 30 min at room temperature and was filtered. The resulting solution was added to a mixture of 1-bromo-3-(fluoromethyl)-5-iodobenzene (60 g, 190.522 mmol), tris (furan-2-yl) phosphane (2.65 g, 11.431 mmol), and Pd₂(dba)₃ (3.49 g, 3.810 mmol) in DMF (400 mL) at room temperature under argon atmosphere and the reaction mixture was heated to 60° C. for 10 min then removed the oil bath. The resulting mixture was stirred for about 1 h until the temperature cooled down to 50° C. The reaction was quenched with aq. NH₄Cl (3000 mL), and the resulting mixture was extracted with EtOAc (3×1000 mL). The combined organic layers were washed with brine (2×1000 mL) and dried over anhydrous Na₂SO₄. After filtration, the filtrate was concentrated under reduced pressure. The residue was purified by silica gel column chromatography (9% EtOAc/pet. ether) to afford the desired product (45 g, 60% yield).
Step 3. A mixture of methyl (2S)-3-[3-bromo-5-(fluoromethyl)phenyl]-2-[(tert-butoxycarbonyl)amino]propanoate (75.28 g, 192.905 mmol), (S)-3-(1-ethyl-2-(2-(1-methoxyethyl)pyridin-3-yl)-5-(4,4,5,5-tetramethyl-1,3,2-dioxaborolan-2-yl)-1H-indol-3-yl)-2,2-dimethylpropan-1-ol (95 g, 192.905 mmol), Pd(dppf)Cl₂ (14.11 g, 19.291 mmol) and K₂CO₃ (53.32 g, 385.810 mmol) in dioxane (900 mL) and H₂O (180 mL) was stirred for 2 h at 80° C. The resulting mixture was concentrated under reduced pressure and was then diluted with H₂O. The resulting mixture was extracted with EtOAc (3×1200 mL), and the combined organic layers were washed with H₂O (3×500 mL) and dried over anhydrous Na₂SO₄. After filtration, the filtrate was concentrated under reduced pressure. The residue was purified by silica gel column chromatography (50% EtOAc/pet. ether) to afford the desired product (105 g, 80% yield). LCMS (ESI) m/z: [M+H] calcd for C₃₉H₅₀FN₃O₆: 676.38; found 676.1.
Step 4. To a stirred solution of methyl (S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)-5-(fluoromethyl)phenyl)propanoate (108 g, 159.801 mmol) in THF (500 mL) was added a solution of LiOH·H₂O (11.48 g, 479.403 mmol) in H₂O (500 mL) at 0° C. The resulting mixture was stirred for 2 h at 0° C. and was then acidified to pH 6 with 1 M HCl (aq.). The mixture was extracted with EtOAc (3×800 mL), and the combined organic layers were washed with brine (2×200 mL) and dried over anhydrous Na₂SO₄. After filtration, the filtrate was concentrated under reduced pressure to afford the desired product (101 g, crude). LCMS (ESI) m/z: [M+H] calcd for C₃₈H₄₈FN₃O₆: 662.36; found 662.1.
Step 5. To a stirred solution of (S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)-5-(fluoromethyl)phenyl)propanoic acid (103 g, 155.633 mmol) and NMM (157.42 g, 1556.330 mmol) in DCM (1200 mL) was added methyl (3S)-1,2-diazinane-3-carboxylate (33.66 g, 233.449 mmol), HOBt (10.51 g, 77.816 mmol) and EDCl (59.67 g, 311.265 mmol) in portions at 0° C. The resulting mixture was stirred a t room temperature for 16 h. The organic layers were then washed with 0.5 M HCl (2×1000 mL) and brine (2×800 mL), dried over anhydrous Na₂SO₄, filtered, and concentrated under reduced pressure. The residue was purified by silica gel column chromatography (50% EtOAc/pet. ether) to afford the desired product (103 g, 83% yield). LCMS (ESI) m/z: [M+H] calcd for C₄₄H₅₈FN₅O₇: 788.44; found 788.1.
Step 6. To a stirred solution of methyl (S)-1-((S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)-5-(fluoromethyl)phenyl)propanoyl)hexahydropyridazine-3-carboxylate (103 g, 130.715 mmol) in THF (700 mL) was added a solution of LiOH·H₂O (27.43 g, 653.575 mmol) in H₂O (700 mL) at 0° C. The resulting mixture was stirred for 2 h at 0° C. and was then neutralized to pH 6 with 1 M HCl. The resulting mixture was extracted with EtOAc (3×800 mL) and the combined organic layers were washed with brine (2×600 mL), dried over anhydrous Na₂SO₄, filtered, and concentrated under reduced pressure to afford the desired product (101 g, crude). LCMS (ESI) m/z: [M+H] calcd for C₄₃H₅₆FN₅O₇: 774.43; found 774.1.
Step 7. To a stirred solution of (S)-1-((S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)-5-(fluoromethyl)phenyl)propanoyl)hexahydropyridazine-3-carboxylic acid (101 g, 130.50 mmol) in DCM (5500 mL) was added DIPEA (227.31 mL, 1305.0 mmol) and HOBt (88.17 g, 652.499 mmol), and EDCl (375.26 g, 1957.498 mmol) at 0° C. The resulting mixture was stirred at room temperature overnight. The mixture was then washed with 0.5 M HCl (2×2000 mL), brine (2×2000 mL), dried over anhydrous Na₂SO₄, filtered, and concentrated under reduced pressure. The residue was purified by silica gel column chromatography (50% EtOAc/pet. ether) to afford the desired product (68 g, 65% yield). LCMS (ESI) m/z: [M+H] calcd for C₄₃H₅₄FN₅O₆: 756.42; found 756.4.
Step 8. To a stirred solution of tert-butyl ((6³S,4S)-1¹-ethyl-2⁵-(fluoromethyl)-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (350 mg, 0.403 mmol) in DCM (4 mL) was added TFA (1.50 mL) at 0° C. The resulting mixture was stirred at room temperature for 1.5 h and was then concentrated under reduced pressure to afford the desired product (600 mg, crude). LCMS (ESI) m/z: [M+H] calcd for C₃₈H₄₆FN₅O₄: 656.36; found 656.4.

### Intermediate 9. Synthesis of methyl (3S)-1-((2S)-3-(5-bromo-3-((tert-butyldiphenylsilyl)oxy)-3,6-dihydropyridin-1(2H)-yl)-2-((tert-butoxycarbonyl)amino)propanoyl)hexahydropyridazine-3-carboxylate

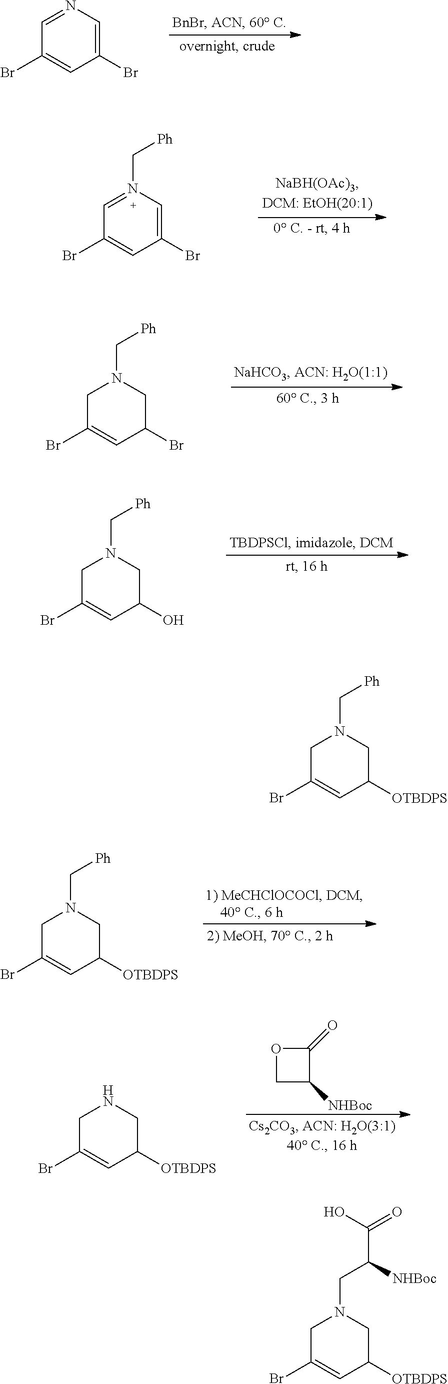

Step 1. To a stirred solution of 3,5-dibromopyridine (48 g, 202.6 mmol, 1 equiv) in ACN (480 mL) were added BnBr (51.9 g, 303.9 mmol, 1.5 equiv) dropwise at room temperature. The resulting mixture was stirred overnight at 60° C. and then concentrated under reduced pressure to afford 1-benzyl-3,5-dibromopyridin-1-ium (76 g, crude) as a white solid. ESI-MS m/z=325.9 [M]⁺, 327.9 [M+2]⁺, 329.9 [M+4]⁺; Calculated MW: 325.9
Step 2. To a stirred mixture of 1-benzyl-3,5-dibromopyridin-1-ium (40 g, 121.9 mmol, 1.00 equiv) in DCM (400 mL) and EtOH (80 mL) was added NaBH(AcO)₃ (129.2 g, 609.7 mmol, 5 equiv) in portions at 0° C. The resulting mixture was stirred for 4 h at room temperature under argon atmosphere after which saturated brine (3×40 mL) was added. The aqueous phase was extracted with CH₂Cl₂ (4×50 mL), dried over anhydrous Na₂SO₄, and filtered. The filtrate was concentrated under reduced pressure and the resulting residue was purified by silica gel column chromatography to afford 1-benzyl-3,5-dibromo-1,2,3,6-tetrahydropyridine (19.1 g, 47.4% yield) as a yellow oil. ESI-MS m/z=330.2 [M+H]⁺, 332.2 [M+H+2]⁺, 334.2 [M+H+4]⁺; Calculated MW: 328.9
Step 3. To a stirred mixture of 1-benzyl-3,5-dibromo-1,2,3,6-tetrahydropyridine (17.4 g, 52.6 mmol, 1.0 equiv) in ACN (87 mL) and H₂O (87 mL) was added NaHCO₃ (22.08 g, 262.800 mmol, 5 equiv) in portions at room temperature. The resulting mixture was stirred for 3 h at 60° C. under argon atmosphere. The mixture was then diluted with brine (300 mL) and the aqueous layer was extracted with EtOAc (4×100 mL). The combined organic layers were concentrated under reduced pressure and the resulting residue was purified by silica gel column chromatography to afford 1-benzyl-5-bromo-1,2,3,6-tetrahydropyridin-3-ol (10 g, 70.9% yield) as a yellow oil. ESI-MS m/z=268.1 [M+H]⁺; Calculated MW: 267.0.
Step 4. To a stirred mixture of Imidazole (5.1 g, 74.6 mmol, 2 equiv) and 1-benzyl-5-bromo-1,2,3,6-tetrahydropyridin-3-ol (10 g, 37.3 mmol, 1.01 equiv) in DCM (100 mL) were added TBDPSCl (15.4 g, 55.9 mmol, 1.5 equiv) dropwise at room temperature. The resulting mixture was stirred for overnight at room temperature under argon atmosphere and then concentrated under reduced pressure. The residue was purified by chromatography to afford 1-benzyl-5-bromo-3-((tert-butyldiphenylsilyl)oxy)-1,2,3,6-tetrahydropyridine (18 g, 95.2% yield) as a yellow oil. ESI-MS m/z=506.4 [M+H]⁺; Calculated MW: 505.1.
Step 5. To a stirred solution of 1-benzyl-5-bromo-3-((tert-butyldiphenylsilyl)oxy)-1,2,3,6-tetrahydropyridine (11.7 g, 23.1 mmol, 1.0 equiv) in DCM (1.2 L) was added 2-chloroethyl chloroformate (13.2 g, 92.4 mmol, 4 equiv) dropwise 0° C. under argon atmosphere. The resulting mixture was stirred for 4 h at 40° C. under argon atmosphere and concentrated under reduced pressure. MeOH (1.2 L) was then added dropwise at room temperature under argon atmosphere and stirred for 2 h at 70° C. under argon atmosphere after which the resulting mixture was concentrated under reduced pressure. The remaining residue was purified by chromatography to afford 5-bromo-3-((tert-butyldiphenylsilyl)oxy)-1,2,3,6-tetrahydropyridine (6.2 g, 64.4% yield) as a yellow oil. ESI-MS m/z=416.2 [M+H]⁺; Calculated MW: 415.1.
Step 6. To a stirred solution of 5-bromo-3-((tert-butyldiphenylsilyl)oxy)-1,2,3,6-tetrahydropyridine (11.2 g, 26.9 mmol, 1.0 equiv) in ACN (90 mL) and H₂O (30 mL) were added tert-butyl(S)-(2-oxooxetan-3-yl) carbamate (6.1 g, 32.4 mmol, 1.2 equiv) and Cs₂CO₃ (21.9 g, 67.5 mmol, 2.5 equiv) at room temperature. The resulting mixture was stirred for 4 h at 40° C. and concentrated under reduced pressure. The remaining residue was purified by chromatography to afford (2S)-3-(5-bromo-3-((tert-butyldiphenylsilyl)oxy)-3,6-dihydropyridin-1(2H)-yl)-2-((tert-butoxycarbonyl)amino)propanoic acid (11.2 g, 68.7% yield) as a white solid. ESI-MS m/z=603.0 [M+H]⁺; Calculated MW: 602.2.
Step 7. To a stirred solution of (2S)-3-(5-bromo-3-((tert-butyldiphenylsilyl)oxy)-3,6-dihydropyridin-1(2H)-yl)-2-((tert-but oxycarbonyl)amino)propanoic acid (5.5 g, 9.2 mmol, 1 equiv) in DCM (55 mL) were added DIEA (47.5 g, 367.1 mmol, 40 equiv), methyl (S)-hexahydropyridazine-3-carboxylate (1.9 g, 13.8 mmol, 1.5 equiv) and CIP (3.3 g, 11.9 mmol, 1.3 equiv) at 0° C. The resulting mixture was stirred for 2 h at 0° C. The mixture was then diluted with brine (500 mL) and the aqueous layer was extracted with DCM (3×400 mL). The combined organic layers were concentrated under reduced pressure and the resulting residue was purified by chromatography to afford methyl (3S)-1-((2S)-3-(5-bromo-3-((tert-butyldiphenylsilyl)oxy)-3,6-dihydropyridin-1(2H)-yl)-2-((tert-butoxycar bonyl)amino)propanoyl)hexahydropyridazine-3-carboxylate (2.2 g, 32.8% yield) as a white solid. ESI-MS m/z=729.1 [M+H]⁺; Calculated MW: 728.3.

### Intermediate 10. Synthesis of (6³S)-4-amino-1¹-ethyl-1²-(2-((S)-1-methoxyethyl)pyridin-3-yl)-10,10-dimethyl-2¹,2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-5,7-dione

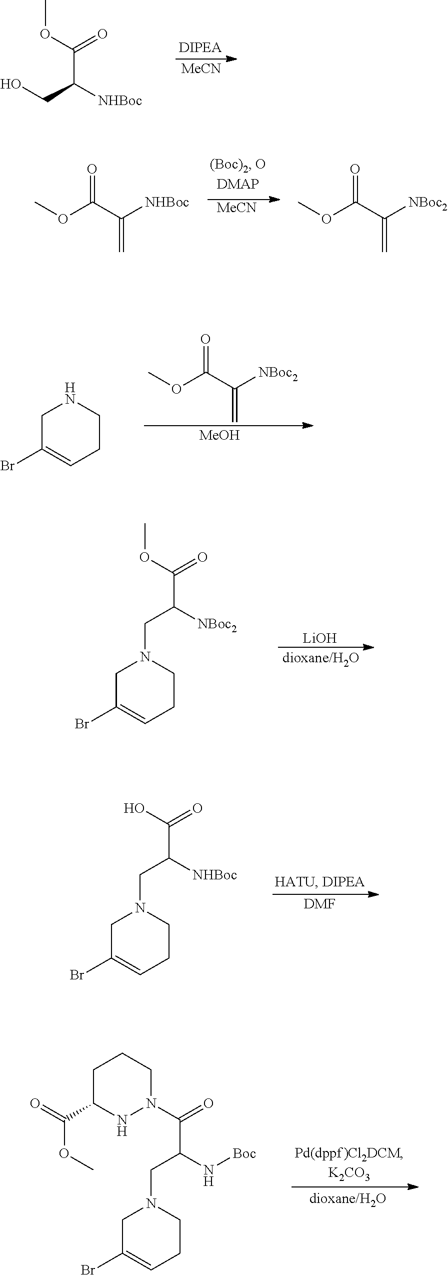

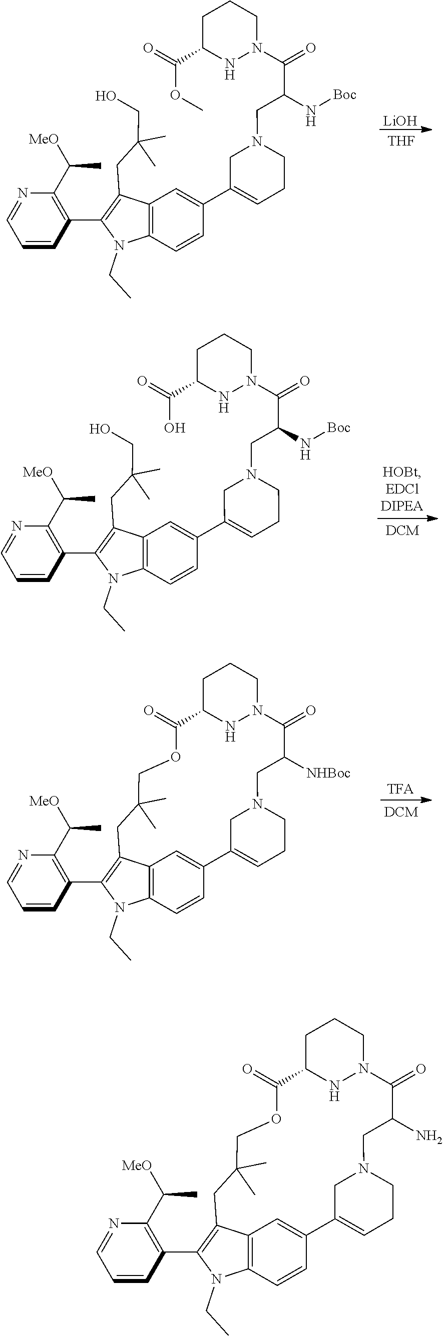

Step 1. To a solution of methyl (tert-butoxycarbonyl)-L-serinate (10 g, 45 mmol) in anhydrous MeCN (150 mL), was added DIPEA (17 g, 137 mmol). The reaction mixture was stirred at 45° C. for 2 h to give the product in solution. LCMS (ESI) m/z [M+Na] calcd for C₉H₁₅NO₄ 201.1; found: 224.1.
Step 2. To a solution of methyl 2-((tert-butoxycarbonyl)amino)acrylate (12 g, 60 mmol) in anhydrous MeCN (150 mL) at 0° C., was added DMAP (13 g, 90 mmol) and (Boc)₂O (26 g, 120 mmol). The reaction was stirred for 6 h, then quenched with H₂O (100 mL) and extracted with DCM (3×200 mL). The combined organic layers were washed with brine (150 mL), dried over anhydrous Na₂SO₄, filtered, and concentrated under reduced pressure. The residue was purified by silica gel column chromatography to give the product (12.5 g, 65% yield) as solid. LCMS (ESI) m/z [M+Na] calcd for C₁₄H₂₃NO₆ 301.2; found: 324.1.
Step 3. To a mixture of 5-bromo-1,2,3,6-tetrahydropyridine (8.0 g, 49 mmol) in MeOH (120 mL) under an atmosphere of Ar was added methyl 2-{bis[(tert-butoxy)carbonyl]amino}prop-2-enoate (22 g, 74 mmol). The mixture was stirred for 16 h, then concentrated under reduced pressure and the residue was purified by silica gel column chromatography to give the product (12 g, 47% yield) as an oil. LCMS (ESI) m/z [M+H] calcd for C₁₉H₃₁BrN₂O₆ 462.1; found: 463.1.
Step 4. To a mixture of methyl 2-(bis (tert-butoxycarbonyl)amino)-3-(5-bromo-3,6-dihydropyridin-1(2H)-yl)propanoate (14 g, 30 mmol) in dioxane (30 mL) and H₂O (12 mL) was added LiOH (3.6 g, 151 mmol). The mixture was heated to 35° C. and stirred for 12 h, then 1M HCl was added, and the pH adjusted to ˜3-4. The mixture was extracted with DCM (2×300 mL) and the combined organic layers were dried over anhydrous Na₂SO₄ and filtered. The filtrate was concentrated under reduced pressure to give the product (10 g, 85% yield) as a solid. LCMS (ESI) m/z [M+H] calcd for C₁₃H₂₁BrN₂O₄ 348.1; found: 349.0.
Step 5. To a mixture of 3-(5-bromo-3,6-dihydropyridin-1(2H)-yl)-2-((tert-butoxycarbonyl)amino)propanoic acid (10 g, 30 mmol), DIPEA (12 g, 93 mmol) and methyl (3S)-1,2-diazinane-3-carboxylate (5.4 g, 37 mmol) in DMF (100 mL) at 0° C. under an atmosphere of Ar was added HATU (13 g, 34 mmol). The mixture was stirred at 0° C. for 2 h, then H₂O was added, and the mixture extracted with EtOAc (2×300 mL). The combined organic layers were dried over anhydrous Na₂SO₄, filtered, the filtrate was concentrated under reduced pressure and the residue was purified by reverse phase chromatography to give the product (9.0 g, 55% yield) as a solid. LCMS (ESI) m/z [M+H] calcd for C₁₉H₃₁BrN₄O₅ 474.1; found: 475.1.
Step 6. A mixture of methyl (3S)-1-(3-(5-bromo-3,6-dihydropyridin-1(2H)-yl)-2-((tert-butoxycarbonyl)amino)propanoyl)hexahydropyridazine-3-carboxylate (9.0 g, 18 mmol), K₂CO₃ (4.5 g, 32 mmol), Pd(dppf)Cl₂·DCM (1.4 g, 2 mmol), 3-(1-ethyl-2-{2-[(1S)-1-methoxyethyl]pyridin-3-yl}-5-(4,4,5,5-tetramethyl-1,3,2-dioxaborolan-2-yl) indol-3-yl)-2,2-dimethylpropan-1-ol (9.8 g, 20 mmol) in dioxane (90 mL) and H₂O (10 mL) under an atmosphere of Ar was heated to 75° C. and stirred for 2 h. H₂O was added and the mixture was extracted with EtOAc (3×200 mL). The combined organic layers were dried over Na₂SO₄, filtered, the filtrate was concentrated under reduced pressure and the residue was purified by silica gel column chromatography to give the product (4.0 g, 25% yield) as a solid. LCMS (ESI) m/z [M+H] calcd for C₄₂H₆₀N₆O₇ 760.5; found: 761.4.
Step 7. To a mixture of methyl (3S)-1-(2-((tert-butoxycarbonyl)amino)-3-(5-(1-ethyl-3-(3-hydroxy-2,2-dimethylpropyl)-2-(2-((S)-1-methoxyethyl)pyridin-3-yl)-1H-indol-5-yl)-3,6-dihydropyridin-1(2H)-yl)propanoyl)hexahydropyridazine-3-carboxylate (4.1 g, 5.0 mmol) in THF (35 mL) at 0° C. was added LiOH (0.60 g, 27 mmol). The mixture was stirred at 0° C. for 1.5 h, then 1M HCl added to adjust pH to ˜6-7 and the mixture was extracted with EtOAc (3×200 mL). The combined organic layers were dried over Na₂SO₄, filtered and the filtrate was concentrated under reduced pressure to give the product (3.6 g, 80% yield) as a solid. LCMS (ESI) m/z [M+H] calcd for C₄₁H₅₈N₆O₇ 746.4; found: 747.4.
Step 8. To a mixture of (3S)-1-(2-((tert-butoxycarbonyl)amino)-3-(5-(1-ethyl-3-(3-hydroxy-2,2-dimethylpropyl)-2-(2-((S)-1-methoxyethyl)pyridin-3-yl)-1H-indol-5-yl)-3,6-dihydropyridin-1(2H)-yl)propanoyl)hexahydropyridazine-3-carboxylic acid (3.6 g, 5.0 mmol) and DIPEA (24 g, 190 mmol) in DCM (700 mL) under an atmosphere of Ar was added EDCl·HCl (28 g, 140 mmol) and HOBt (6.5 g, 50 mmol). The mixture was heated to 30° C. and stirred for 16 h at 30° C., then concentrated under reduced pressure. The residue was diluted with EtOAc (200 mL) and washed with H₂O (2×200 mL), brine (200 mL), dried over Na₂SO₄ and filtered. The filtrate was concentrated under reduced pressure and the residue was purified by silica gel column chromatography to give the product (1.45 g, 40% yield) as a solid. LCMS (ESI) m/z [M+H] calcd for C₄₁H₅₆N₆O₆ 728.4; found: 729.4.
Step 9, To a mixture of tert-butyl ((6³S)-1¹-ethyl-1²-(2-((S)-1-methoxyethyl)pyridin-3-yl)-10,10-dimethyl-5,7-dioxo-2¹,2²,2³,2⁶,6¹,6²,6³,6⁴,6⁵,6⁶-decahydro-1¹H-8-oxa-1(5,3)-indola-6(1,3)-pyridazina-2(5,1)- pyridinacycloundecaphane-4-yl)carbamate (130 mg, 0.20 mmol) in DCM (1.0 mL) at 0° C. was added TFA (0.3 mL). The mixture was warmed to room temperature and stirred for 2 h, then concentrated under reduced pressure to give the product, which was used directly in the next step directly without further purification. LCMS (ESI) m/z [M+H] calcd for C₃₆H₄₈N₆O₄ 628.4; found: 629.4.

### Intermediate 11. Synthesis of (6³S,4S)-4-amino-1¹-ethyl-1²-(2-((S)-1-methoxyethyl)-5-((R)-octahydro-2H-pyrido[1,2-a]pyrazin-2-yl)pyridin-3-yl)-10,10-dimethyl-2¹,2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-5,7-dione

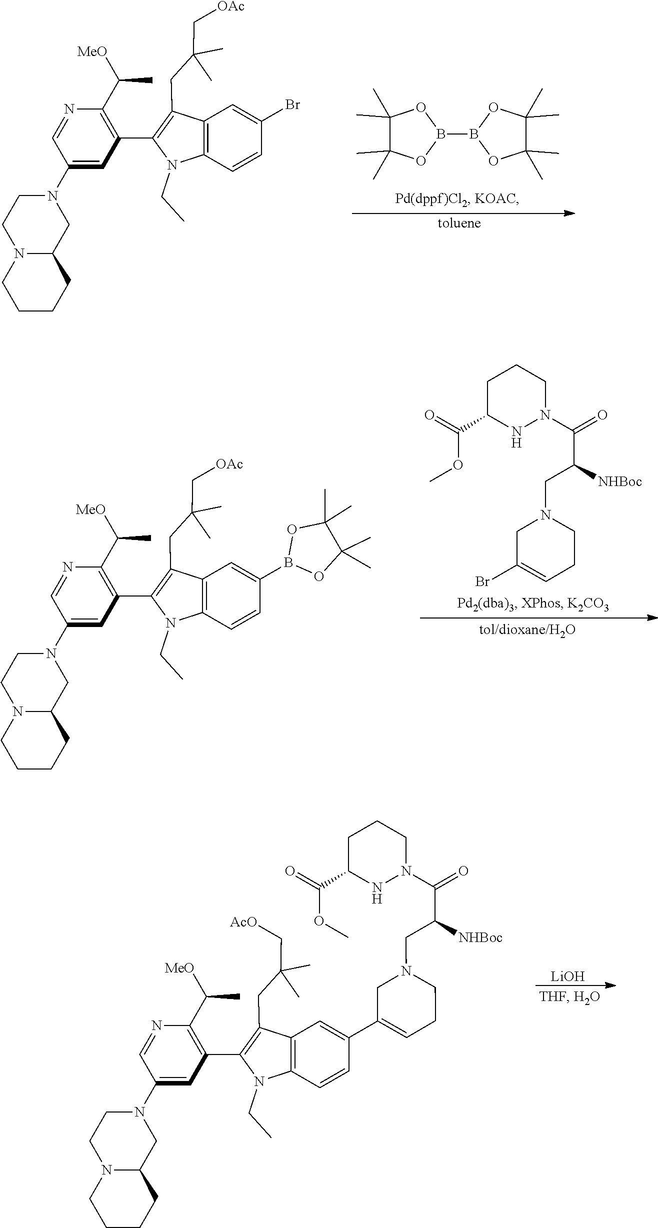

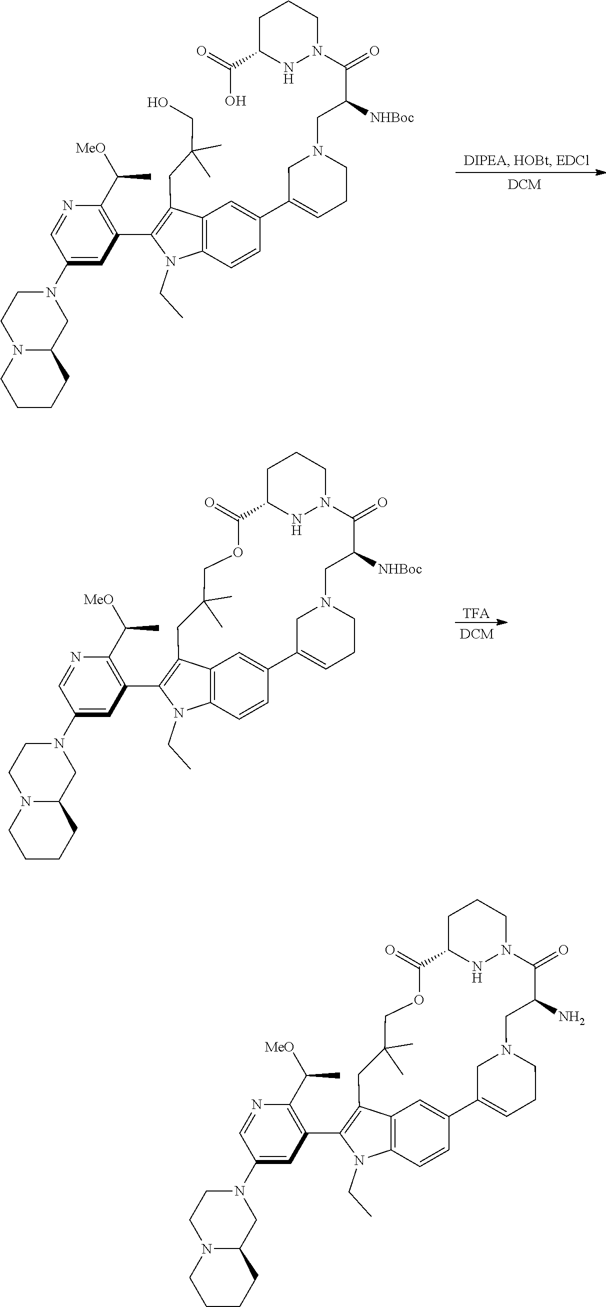

Step 1. To a stirred solution of 3-(5-bromo-1-ethyl-2-(2-((S)-1-methoxyethyl)-5-((R)-octahydro-2H-pyrido[1,2-a]pyrazin-2-yl)pyridin-3-yl)-1H-indol-3-yl)-2,2-dimethylpropyl acetate (1 g, 1.598 mmol) and B₂Pin₂ (0.81 g, 3.196 mmol) in toluene (20 mL) was added KOAc (0.39 g, 3.995 mmol) and Pd(dppf)Cl₂ (0.12 g, 0.16 mmol). The mixture was stirred for 2 h at 90° C. under a nitrogen atmosphere. The mixture was then basified to pH 8 with sat. aq. NaHCO₃. The resulting mixture was extracted with DCM (3×40 mL) and the combined organic layers were washed with brine (3×40 mL) and dried over anhydrous Na₂SO₄. After filtration, the filtrate was concentrated under reduced pressure. The residue was purified by silica gel column chromatography (2% MeOH/DCM) to afford the product (0.9 g, 83% yield) as a solid. LCMS (ESI) m/z [M+H] calcd for C₃₉H₅₇BN₄O₅: 673.45; found: 673.6
Step 2. To a stirred solution of 3-(1-ethyl-2-(2-((S)-1-methoxyethyl)-5-((R)-octahydro-2H-pyrido[1,2-a]pyrazin-2-yl)pyridin-3-yl)-5-(4,4,5,5-tetramethyl-1,3,2-dioxaborolan-2-yl)-1H-indol-3-yl)-2,2-dimethylpropyl acetate (0.9 g, 1.338 mmol), methyl (3S)-1-[(2S)-3-(3-bromo-5,6-dihydro-2H-pyridin-1-yl)-2-[(tert-butoxycarbonyl)amino]propanoyl]-1,2-diazinane-3-carboxylate (1.02 g, 2.141 mmol), K₂CO₃ (0.46 g, 3.345 mmol), and X-Phos (0.26 g, 0.535 mmol) in toluene (13.5 mL), dioxane (90 mL), and H₂O (4.5 mL) was added Pd₂(dba)₃ (0.37 g, 0.401 mmol). The mixture was stirred for 2 h at 70° C. under a nitrogen atmosphere. The mixture was then basified to pH 8 with sat. aq. NaHCO₃. The resulting mixture was extracted with DCM (3×100 mL) and the combined organic layers were washed with brine (3×100 mL) and dried over anhydrous Na₂SO₄. After filtration, the filtrate was concentrated under reduced pressure. The residue was purified by silica gel column chromatography (2% MeOH/DCM) to afford the product (1.1 g, 87% yield) as a solid. LCMS (ESI) m/z [M+H] calcd for C₅₂H₇₆N₈O₈: 941.59; found: 941.8
Step 3. To a stirred solution of methyl (S)-1-((S)-3-(5-(3-(3-acetoxy-2,2-dimethylpropyl)-1-ethyl-2-(2-((S)-1-methoxyethyl)-5-((R)-octahydro-2H-pyrido[1,2-a]pyrazin-2-yl)pyridin-3-yl)-1H-indol-5-yl)-3,6-dihydropyridin-1(2H)-yl)-2-((tert-butoxycarbonyl)amino)propanoyl)hexahydropyridazine-3-carboxylate (1.1 g, 1.169 mmol) in THF (8 mL) was added a solution of LiOH (0.14 g, 5.845 mmol) in H₂O (8 mL) dropwise at 0° C. under a nitrogen atmosphere. The reaction mixture was stirred for 16 h. The mixture was then acidified to pH 6 with conc. HCl. The resulting mixture was extracted with DCM (3×50 mL) and dried over anhydrous Na₂SO₄. After filtration, the filtrate was concentrated under reduced pressure to afford the product (1.0 g, 96% yield) as a solid, which was used in the next step directly without further purification. LCMS (ESI) m/z [M+H] calcd for C₄₉H₇₂N₈O₇: 885.56; found: 885.5
Step 4. To a stirred solution of (S)-1-((S)-2-((tert-butoxycarbonyl)amino)-3-(5-(1-ethyl-3-(3-hydroxy-2,2-dimethylpropyl)-2-(2-((S)-1-methoxyethyl)-5-((R)-octahydro-2H-pyrido[1,2-a]pyrazin-2-yl)pyridin-3-yl)-1H-indol-5-yl)-3,6-dihydropyridin-1(2H)-yl)propanoyl)hexahydropyridazine-3-carboxylic acid (1.0 g, 1.13 mmol) and HOBt (0.76 g, 5.65 mmol) in DCM (100 mL) was added EDC·HCl (6.06 g, 31.64 mmol) and DIPEA (5.11 g, 39.55 mmol) dropwise at 0° C. under a nitrogen atmosphere. The reaction mixture was stirred for 16 h. The mixture was then basified to pH 8 with sat. aq. NaHCO₃. The resulting mixture was extracted with DCM (3×100 mL) and the combined organic layers were washed with brine (3×100 mL) and dried over anhydrous Na₂SO₄. After filtration, the filtrate was concentrated under reduced pressure. The residue was purified by silica gel column chromatography (3% MeOH/DCM) to afford the product (650 mg, 66% yield) as a solid. LCMS (ESI) m/z [M+H] calcd for C₄₉H₇₀N₈O₆: 867.55; found: 867.5
Step 5. To a stirred solution of tert-butyl ((6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-2¹,2²,2³,2⁶,6¹,6²,6³,6⁴,6⁵,6⁶-decahydro-1¹H-8-oxa-1(5,3)-indola-6(1,3)-pyridazina-2(5, 1)-pyridinacycloundecaphane-4-yl)carbamate (300 mg, 0.346 mmol) in DCM (3 mL) was added TFA (3 mL) dropwise at 0° C. under a nitrogen atmosphere. The resulting mixture was stirred for 1 h at 0° C. The mixture was then basified to pH 8 with sat. aq. NaHCO₃. The resulting mixture was extracted with DCM (3×50 mL) and the combined organic layers were washed with brine (3×50 mL) and dried over anhydrous Na₂SO₄. After filtration, the filtrate was concentrated under reduced pressure to afford the product (260 mg, 98% yield) as a solid, which was used in the next step directly without further purification. LCMS (ESI) m/z [M+H] calcd for C₄₄H₆₂N₈O₄: 767.50; found: 767.2.

### Intermediate 12. Synthesis of (2²S,6³S,4S)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-2(4,2)-morpholina-1(5,3)-indola-6(1,3)-pyridazinacycloundecaphane-5,7-dione

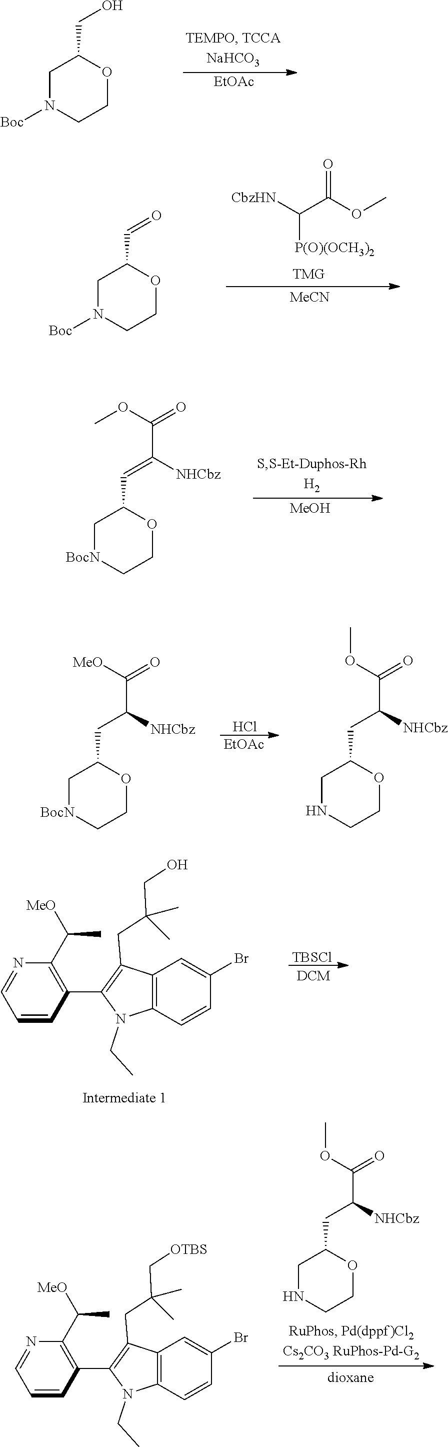

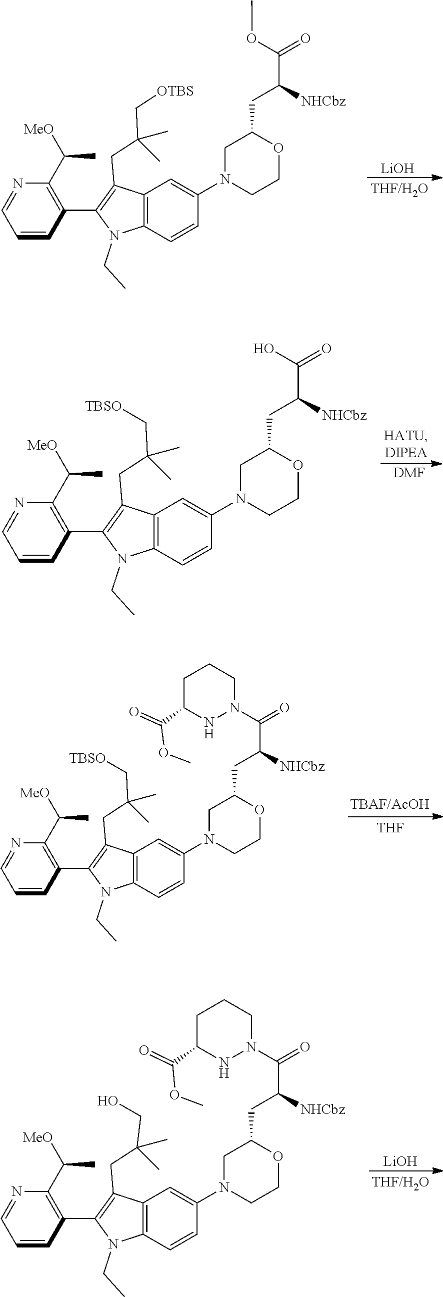

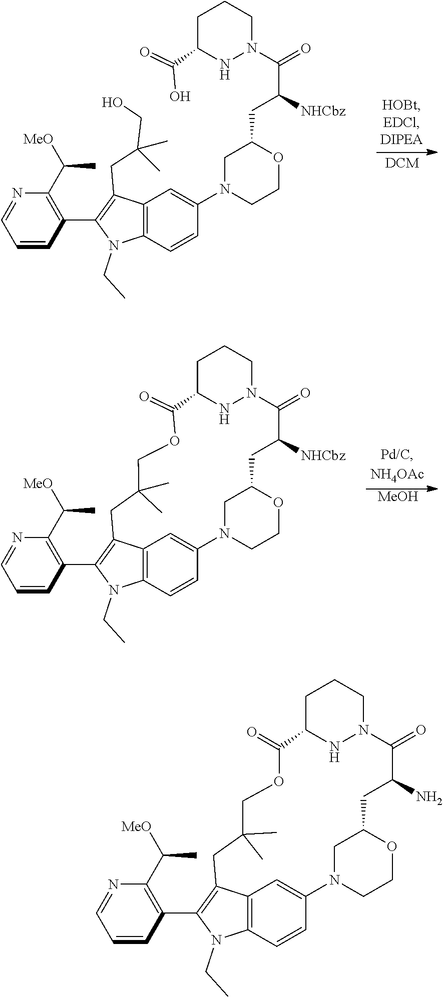

Step 1. To a solution of tert-butyl (2R)-2-(hydroxymethyl) morpholin-4-yl formate (50 g, 230 mmol) in EtOAc (1 L) was added TEMPO (715 mg, 4.6 mmol) and NaHCO₃ (58 g, 690 mmol) at room temperature. The mixture was cooled to −50° C., then TCCA (56 g, 241 mmol) in EtOAc (100 mL) was added dropwise over 30 min. The reaction mixture was warmed to 5° C. for 2 h, then quenched with 10% Na₂S₂O₃ (200 mL) and stirred for 20 min. The resulting mixture was filtered, and the organic phase was separated. The aqueous phase was extracted with EtOAc (2×100 mL). The combined organic layers were washed with H₂O (100 mL) and brine (100 mL), then dried over anhydrous Na₂SO₄. The organic layer was concentrated under reduced pressure to afford the product (50 g, crude) as an oil.
Step 2. To a solution of tert-butyl (2R)-2-formylmorpholin-4-yl formate (49 g, 153 mmol) and methyl 2-{[(benzyloxy)carbonyl]amino}-2-(dimethoxyphosphoryl) acetate (60 g, 183 mmol) in MeCN (300 mL) was added tetramethylguanidine (35 g, 306 mmol) at 0-10° C. The reaction mixture was stirred at 10° C. for 30 min then warmed to room temperature for 2 h. The reaction mixture was diluted with DCM (200 mL) and washed with 10% citric acid (200 mL) and 10% NaHCO₃ aq. (200 mL). The organic phase was concentrated under reduced pressure and purified by silica gel column chromatography to afford the product (36 g, 90% yield) as solid. LCMS (ESI) m/z [M+Na] calcd for C₂₁H₂₈N₂O₄ 420.2; found: 443.1
Step 3. To a solution of tert-butyl (S,Z)-2-(2-(((benzyloxy)carbonyl)amino)-3-methoxy-3-oxoprop-1-en-1-yl) morpholine-4-carboxylate (49 g, 0.12 mol) in MeOH (500 mL) was added (S,S)-Et-DUPHOS-Rh (500 mg, 0.7 mmol). The mixture was stirred at room temperature under an H₂ (60 psi) atmosphere for 48 h. The reaction was concentrated and purified by silica gel column chromatography to give the product (44 g, 90% yield) as solid. LCMS (ESI) m/z [M+Na] calcd for C₂₁H₃₀N₂O₇ 422.2; found: 445.2.
Step 4. To a stirred solution of tert-butyl(S)-2-((S)-2-(((benzyloxy)carbonyl)amino)-3-methoxy-3-oxopropyl) morpholine-4-carboxylate (2.2 g, 5.2 mmol) in EtOAc (2 mL) was added HCl/EtOAc (25 mL) at 15° C. The reaction was stirred at 15° C. for 2 h, then concentrated under reduced pressure to afford the product (1.51 g, 90% yield) as an oil. LCMS (ESI) m/z [M+H] calcd for C₁₆H₂₂N₂O₅ 322.1; found: 323.2.
Step 5. To a solution of 3-(5-bromo-1-ethyl-2-{2-[(1S)-1-methoxyethyl]pyridin-3-yl}indol-3-yl)-2,2-dimethylpropan-1-ol (100 g, 0.22 mol) and imidazole (30.6 g, 0.45 mol) in DCM (800 mL) was added TBSCl (50.7 g, 0.34 mol) in DCM (200 mL) at 0° C. The reaction was stirred at room temperature for 2 h. The resulting solution was washed with H₂O (3×300 mL) and brine (2×200 mL), dried over anhydrous Na₂SO₄, filtered, and concentrated under reduced pressure. The residue was purified with silica gel column chromatography to give the product (138 g, 90% yield) as a solid. LCMS (ESI) m/z [M+H] calcd for C₂₉H₄₃BrN₂O₂Si 558.2; found: 559.2.
Step 6. To a stirred solution of (S)-5-bromo-3-(3-((tert-butyldimethylsilyl)oxy)-2,2-dimethylpropyl)-1-ethyl-2-(2-(1-methoxyethyl)pyridin-3-yl)-1H-indole (50 g, 89.3 mmol) in dioxane (500 mL) was added methyl (2S)-2-{[(benzyloxy)carbonyl]amino}-3-[(2S)-morpholin-2-yl]propanoate (31.7 g, 98.2 mmol), RuPhos (16.7 g, 35.7 mmol), di-μ-chlorobis(2-amino-1,1-biphenyl-2-yl-C,N)dipalladium(II) (2.8 g, 4.4 mmol) and cesium carbonate (96 g, 295 mmol) followed by RuPhos-Pd-G2 (3.5 g, 4.4 mmol) at 105° C. under an N₂ atmosphere. The reaction mixture was stirred for 6 h at 105° C. under an N₂ atmosphere. The resulting mixture was filtered, and the filtrate was concentrated under reduced pressure. The residue was purified by prep-TLC chromatography to afford the product (55 g, 73% yield) as a solid. LCMS (ESI) m/z [M+H] calcd for C₄₅H₆₄N₄O₇Si 800.5; found: 801.5.
Step 7. To a solution of methyl (2S)-2-{[(benzyloxy)carbonyl]amino}-3-[(2S)-4-(3-{3-[(tert-butyldimethylsilyl)oxy]-2,2-dimethylpropyl}-1-ethyl-2-{2-[(1S)-1-methoxyethyl]pyridin-3-yl}indol-5-yl) morpholin-2-yl]propanoate (10 g, 12 mmol) in THF (270 mL) was added LiOH (1.3 g, 31 mmol) in H₂O (45 mL) at room temperature. The reaction was stirred at room temperature for 2 h, then treated with 1N HCl to adjust pH to 4˜5 at 0˜5° C. The resulting mixture was extracted with EtOAc (2×50 mL). The combined organic layers were washed with brine and dried over anhydrous Na₂SO₄. The organic phase was then concentrated under reduced pressure to afford the product (9.5 g, 97% yield) as a solid. LCMS (ESI) m/z [M+H] calcd for C₄₄H₆₂N₄O₇Si 786.4; found: 787.4.
Step 8. To a stirred solution of (2S)-2-{[(benzyloxy)carbonyl]amino}-3-[(2S)-4-(3-{3-[(tert-butyldimethylsilyl)oxy]-2,2-dimethylpropyl}-1-ethyl-2-{2-[(1S)-1-methoxyethyl]pyridin-3-yl}indol-5-yl)morpholin-2-yl]propanoic acid (10 g, 12.7 mmol) in DMF (150 mL), was added methyl (S)-hexahydropyridazine-3-carboxylate (2 g, 14 mmol), then cooled to 0° C., DIPEA (32.8 g, 254 mmol) was added followed by HATU (9.7 g, 25.4 mmol) at 0˜5° C. The reaction mixture was stirred at 0˜5° C. for 1 h. The resulting mixture was diluted with EtOAc (500 mL) and H₂O (200 mL). The organic layer was separated and washed with H₂O (2×100 mL) and brine (100 mL), dried over anhydrous sodium sulfate. The solution was filtered and concentrated under reduced pressure, and the residue was purified by silica gel column chromatography to afford the product. LCMS (ESI) m/z [M+H] calcd for C₅₀H₇₂N₆O₈Si 912.5; found: 913.4.
Step 9. A solution of methyl (S)-1-((S)-2-(((benzyloxy)carbonyl)amino)-3-((S)-4-(3-(3-((tert-butyldimethylsilyl)oxy)-2,2-dimethylpropyl)-1-ethyl-2-(2-((S)-1-methoxyethyl)pyridin-3-yl)-1H-indol-5-yl) morpholin-2-yl)propanoyl)hexahydropyridazine-3-carboxylate (8.5 g, 9 mmol) in THF (8 mL) was added a mixture of tetrabutylammonium fluoride (1M in THF, 180 mL, 180 mmol) and AcOH (11 g, 200 mmol) at room temperature. The reaction mixture was stirred at 75° C. for 3 h. The resulting mixture was diluted with EtOAc (150 mL) and washed with H₂O (6×20 mL). The organic phase was concentrated under reduced pressure to give the product (7.4 g, 100% yield) as solid. LCMS (ESI) m/z [M+H] calcd for C₄₄H₅₈N₆O₈ 799.4; found: 798.4.
Step 10. To a solution of methyl (S)-1-((S)-2-(((benzyloxy)carbonyl)amino)-3-((S)-4-(1-ethyl-3-(3-hydroxy-2,2-dimethylpropyl)-2-(2-((S)-1-methoxyethyl)pyridin-3-yl)-1H-indol-5-yl) morpholin-2-yl)propanoyl)hexahydropyridazine-3-carboxylate (8 g, 10 mmol) in THF (200 mL) was added LiOH (600 mg, 25 mmol) in H₂O (30 mL). The reaction mixture was stirred at room temperature for 1 h, then treated with 1N HCl to adjust pH to 4˜5 at 0˜5° C. and extracted with EtOAc (2×500 mL). The organic phase was washed with brine and concentrated under reduced pressure to afford the product (8 g, crude) as a solid. LCMS (ESI) m/z [M+H] calcd for C₄₃H₅₆N₆O₈ 784.4; found: 785.4.
Step 11. To a stirred solution of (S)-1-((S)-2-(((benzyloxy)carbonyl)amino)-3-((S)-4-(1-ethyl-3-(3-hydroxy-2,2-dimethylpropyl)-2-(2-((S)-1-methoxyethyl)pyridin-3-yl)-1H-indol-5-yl) morpholin-2-yl)propanoyl)hexahydropyridazine-3-carboxylic acid (8 g, 10.2 mmol) and DIPEA (59 g, 459 mmol) in DCM (800 mL) was added EDCl (88 g, 458 mmol) and HOBt (27.6 g, 204 mmol) at room temperature under an argon atmosphere. The reaction mixture was stirred at room temperature for 16 h. The resulting mixture was concentrated under reduced pressure, and the residue was purified by silica gel column chromatography to afford the product (5 g, 66% yield) as a solid; LCMS (ESI) m/z [M+H] calcd for C₄₃H₅₄N₆O₇ 766.4; found: 767.4.
Step 12. To a solution of benzyl ((2²S,6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)carbamate (400 mg, 0.5 mmol) in MeOH (20 mL) was added Pd/C (200 mg) and ammonium acetate (834 mg, 16 mmol) at room temperature under an H₂ atmosphere and the mixture was stirred for 2 h. The resulting mixture was filtered and concentrated under reduced pressure. The residue was redissolved in DCM (20 mL) and washed with H₂O (5 mL×2), then concentrated under reduced pressure to afford the product (320 mg, 97% yield) as a solid. LCMS (ESI) m/z [M+H] calcd for C₃₅H₄₈N₆O₅ 632.4; found: 633.3.

### Intermediate 13. Synthesis of tert-butyl ((6³S,4S)-1²-(2-((S)-1-methoxyethyl)pyridin-3-yl)-10,10-dimethyl-5,7-dioxo-1¹-(2,2,2-trifluoroethyl)-2¹,2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-4-yl)carbamate

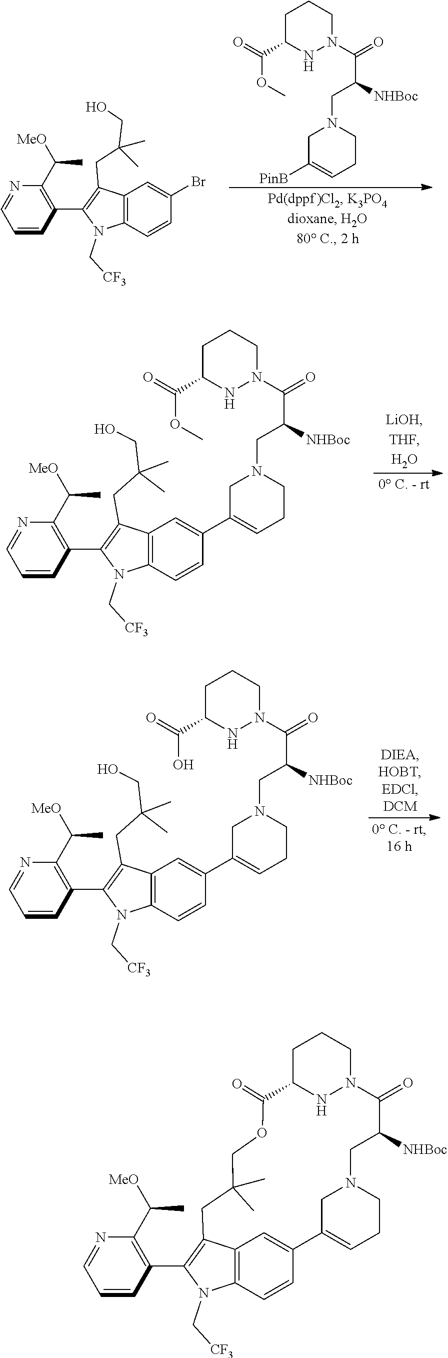

Step 1. To a stirred solution of 3-(5-bromo-2-(2-(methoxymethyl)pyridin-3-yl)-1-(2,2,2-trifluoroethyl)-1H-indol-3-yl)-2,2-dimethylpropan-1-ol (100 g, 200.3 mmol, 1 equiv) and methyl (3S)-1-[(2S)-2-[(tert-butoxycarbonyl)amino]-3-[3-(4,4,5,5-tetramethyl-1,3,2-dioxaborolan-2-yl)-5,6-dihydro-2H-pyridin-1-yl]propanoyl]-1,2-diazinane-3-carboxylate (132.3 g, 300.4 mmol, 1.5 equiv) in dioxane (1000 mL) was added Pd(dppf)Cl₂ (14.65 g, 20.025 mmol, 0.1 equiv) and K₃PO₄ (106.27 g, 500.630 mmol, 2.5 equiv) in H₂O (200 mL) in portions at 80° C. under argon atmosphere. The resulting mixture was stirred for 2 h at 80° C. under argon atmosphere. After which the resulting mixture was extracted with DCM/MeOH (5/1; 3×1000 mL). The combined organic layers were washed with brine (2×1000 mL), dried over anhydrous Na₂SO₄. After filtration, the filtrate was concentrated under reduced pressure. The crude product (180 g, crude) was used in the next step directly without further purification. ESI-MS m/z=815.1 [M+H]⁺; Calculated MW: 814.4.
Step 2. To a stirred solution of methyl (S)-1-((S)-2-((tert-butoxycarbonyl)amino)-3-(5-(3-(3-hydroxy-2,2-dimethylpropyl)-2-(2-((S)-1-methoxyethyl)pyridin-3-yl)-1-(2,2,2-trifluoroethyl)-1H-indol-5-yl)-3,6-dihydropyridin-1(2H)-yl)propanoyl)hexahydropyridazine-3-carboxylate (180 g, 220.9 mmol, 1 equiv) in THF (1500 mL) was added LiOH·H₂O (46.4 g, 1104.4 mmol, 5 equiv) in H₂O (300 mL) dropwise at 0° C. and stirred for 2 h at 25° C. under N₂ atmosphere. The mixture was acidified to pH=6 with HCl (1N). The resulting mixture was extracted with DCM/MeOH (5/1; 3×1500 ml). The combined organic layers were washed with brine (3×1000 ml) and dried over anhydrous Na₂SO₄. After filtration, the filtrate was concentrated under reduced pressure. The crude product (180 g, crude) was used in the next step directly without further purification. ESI-MS m/z=801.3 [M+H]⁺; Calculated MW: 800.4.
Step 3. To a stirred solution of (S)-1-((S)-2-((tert-butoxycarbonyl)amino)-3-(5-(3-(3-hydroxy-2,2-dimethylpropyl)-2-(2-((S)-1-methoxyethyl)pyridin-3-yl)-1-(2,2,2-trifluoroethyl)-1H-indol-5-yl)-3,6-dihydropyridin-1(2H)-yl)propanoyl)hexahydropyridazine-3-carboxylic acid (200 g, 249.7 mmol, 1 equiv) and DIEA (968.2 g, 7491.4 mmol, 30 equiv) in DCM (20 L) was added HOBT (168.7 g, 1248.6 mmol, 5 equiv) and EDCl (1340.37 g, 6991.964 mmol, 28 equiv) in portions at 0° C. under nitrogen atmosphere. The resulting mixture was stirred for 16 h at 25° C. under nitrogen atmosphere. The mixture was acidified to pH 6-7 with HCl (1N). The resulting mixture was extracted with EA (3×2000 ml). The combined organic layers were washed with brine (3×1500 ml) and dried over anhydrous Na₂SO₄. After filtration, the filtrate was concentrated under reduced pressure. The residue was purified by silica gel column chromatography to afford tert-butyl ((6³S,4S)-1²-(2-((S)-1-methoxyethyl)pyridin-3-yl)-10,10-dimethyl-5,7-dioxo-1¹-(2,2,2-trifluoroethyl)-2¹,2²,2³,2⁶,6¹,6²,6³,6⁴,6⁵,6⁶- decahydro-1¹H-8-oxa-1(5,3)-indola-6(1,3)-pyridazina-2(5,1)-pyridinacycloundecaphane-4-yl)carbamate (100 g, 63.7% yield over three steps) as a yellow solid. ESI-MS m/z=783.3 [M+H]⁺; Calculated MW: 782.4.

### Intermediate 14. Synthesis of tert-butyl ((6³S,4S)-1¹-ethyl-10,10-difluoro-1²-(2-((S)-1-methoxyethyl)pyridin-3-yl)-5,7-dioxo-2¹,2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-4-yl)carbamate

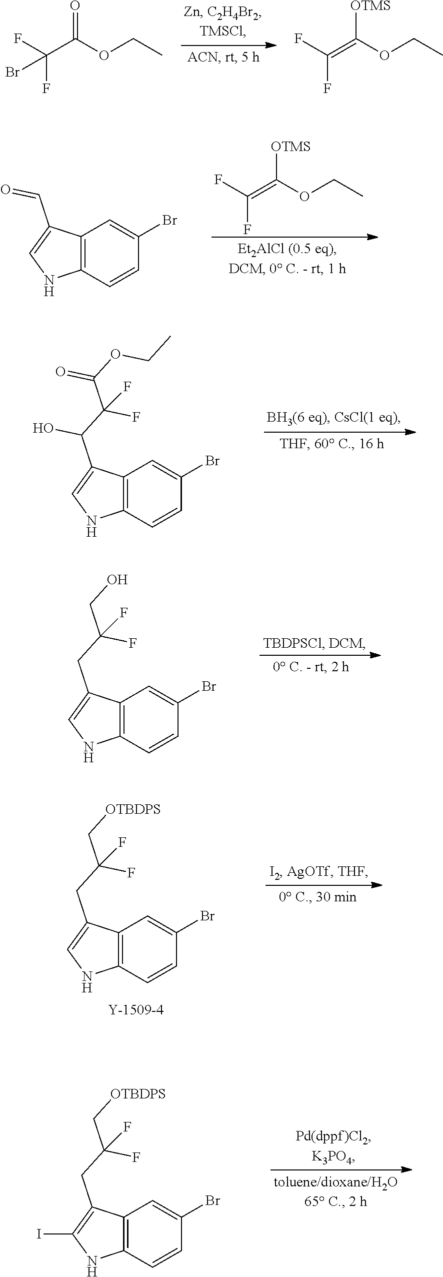

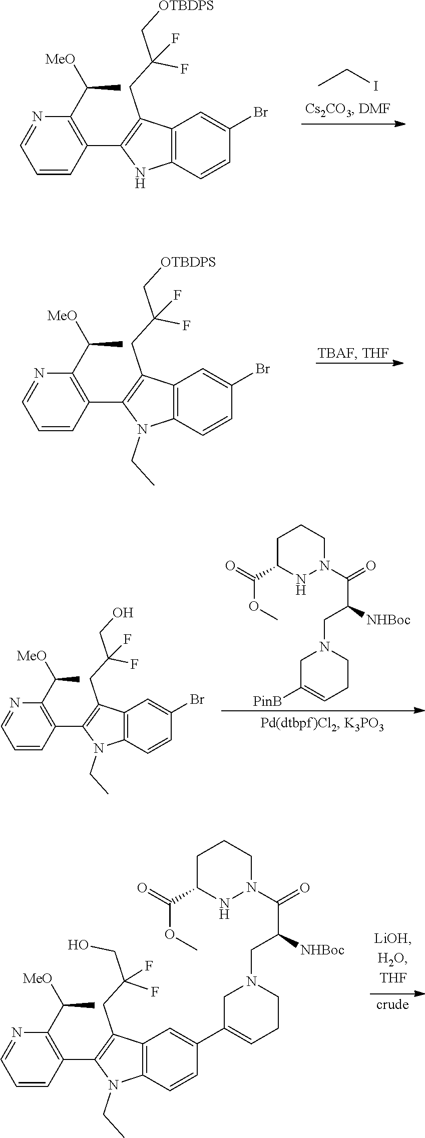

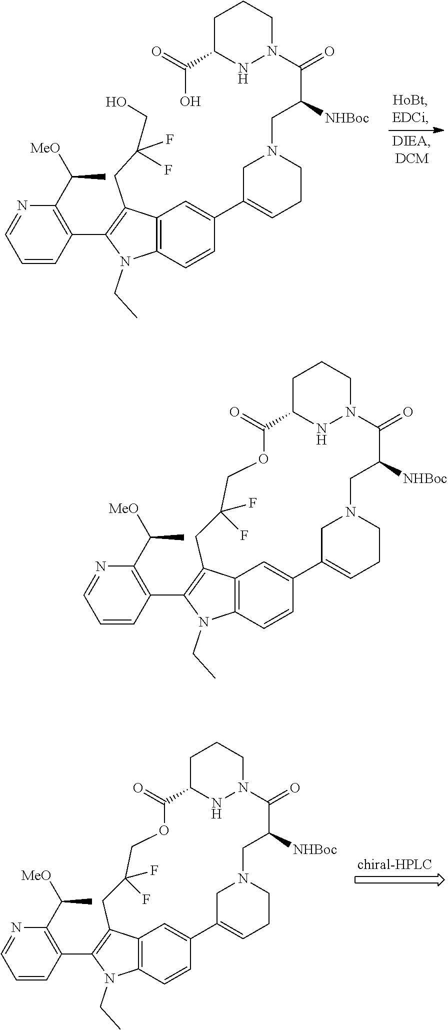 [SMILES: CCn1c(-c2cccnc2[C@H](C)OC)c(CC(F)(F)CO)c2cc(C3=CCCN(C[C@H](NC(=O)OC(C)(C)C)C(=O)N4CCC[C@@H](C(=O)O)N4)C3)ccc21 ; CCn1c(-c2cccnc2[C@H](C)OC)c2c3cc(ccc31)C1=CCCN(C1)C[C@H](NC(=O)OC(C)(C)C)C(=O)N1CCC[C@H](N1)C(=O)OCC(F)(F)C2 ; CCn1c(-c2cccnc2[C@H](C)OC)c2c3cc(ccc31)C1=CCCN(C1)C[C@H](NC(=O)OC(C)(C)C)C(=O)N1CCC[C@H](N1)C(=O)OCC(F)(F)C2]

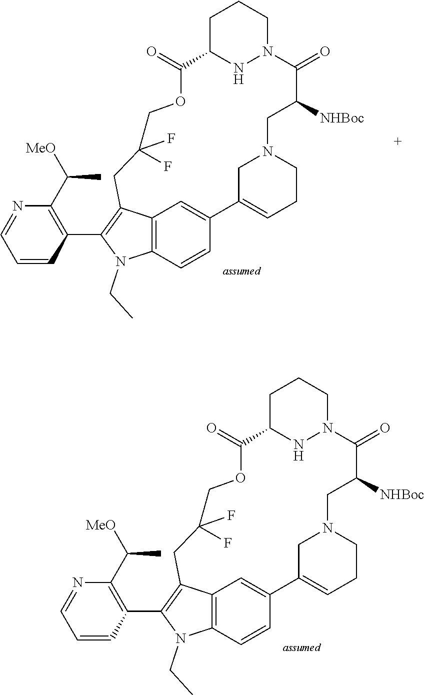

Step 1. A round-bottom flask was charged with Zn (16.4 g, 251.3 mmol, 1.7 equiv) at room temperature. The resulting mixture was stirred for 1 min at room temperature under argon atmosphere. ACN (120 mL) was then added in portions over 1 min at room temperature. The resulting mixture was stirred for additional 5 min at room temperature after which C₂H₄Br₂ (30.2 g, 162.5 mmol, 1.1 equiv) was added dropwise over 5 min at room temperature and the resulting mixture was stirred for 1 min at room temperature. TMSCl (28.9 g, 266.0 mmol, 1.8 equiv) was then added dropwise over 30 min at 0° C. The resulting mixture was stirred for additional 5 min at room temperature. Ethyl 2-bromo-2,2-difluoroacetate (30 g, 147.8 mmol, 1.0 equiv) was then added dropwise to the reaction mixture over 10 min at room temperature. The resulting mixture was stirred for additional 1h at room temperature. TMSCl (24.1 g, 221.7 mmol, 1.5 equiv) was then added dropwise over 5 min at room temperature. The resulting mixture was stirred for additional 1h at room temperature. The reaction mixture was diluted with Heptane (110 mL, 1097.7 mmol, 7.4 equiv) dropwise over 2 min at room temperature and stirred for 30 mins at room temperature. The mixture was used in the next step directly without further purification.
Step 2. To a stirred solution of 5-bromo-1H-indole-3-carbaldehyde (10 g, 44.6 mmol, 1.0 equiv) in DCM (200 mL) was added Et₂AlCl (22.3 mL, 22.3 mmol, 0.5 equiv) dropwise at 0° C. under argon atmosphere. After which [(1-ethoxy-2,2-difluoroethenyl)oxy]trimethylsilane (205.9 mL, 267.7 mmol, 6 equiv) dropwise over 5 min at 0° C. and the resulting mixture was stirred for additional 1h at room temperature. The mixture was then extracted with EtOAc (3×100 mL), and the combined organic layers were washed with brine (3×60 mL), dried over anhydrous Na₂SO₄, and filtered. The filtrate was concentrated under reduced pressure and the remaining residue was purified by silica gel column chromatography to afford ethyl 3-(5-bromo-1H-indol-3-yl)-2,2-difluoro-3-hydroxypropanoate (3.5 g, 64% yield) as a yellow oil. ESI-MS m/z=347.9 [M+H]⁺; Calculated MW: 347.0.
Step 3. To a stirred solution of ethyl 3-(5-bromo-1H-indol-3-yl)-2,2-difluoro-3-hydroxypropanoate (3.5 g, 10.0 mmol, 1.0 equiv) and CsCl (1.69 g, 10.053 mmol, 1 equiv) in THF (35 mL) were added BH₃·THF (60.3 mL, 60.3 mmol, 6 equiv) dropwise at room temperature under air atmosphere. The resulting mixture was stirred overnight at 65° C. under air atmosphere. The reaction was quenched with sat. NaHCO₃(aq.) at room temperature, and the resulting mixture was extracted with EtOAc (3×20 mL). The combined organic layers were washed with brine (3×10 mL), dried over anhydrous Na₂SO₄, and filtered. The filtrate was concentrated under reduced pressure to afford 3-(5-bromo-1H-indol-3-yl)-2,2-difluoropropan-1-ol (3.6 g, crude) as a yellow oil. ESI-MS m/z=288.0 [M−H]⁺; Calculated MW: 289.0.
Step 4. To a stirred solution of 3-(5-bromo-1H-indol-3-yl)-2,2-difluoropropan-1-ol (3.5 g, 12.0 mmol, 1.0 equiv) and Imidazole (2.0 g, 30.1 mmol, 2.5 equiv) in DCM (40 mL) was added TBDPSCl (4.3 g, 15.6 mmol, 1.3 equiv) dropwise at 0° C. under air atmosphere. The resulting mixture was stirred for 2 h at room temperature after which the mixture was extracted with CH₂Cl₂ (3×20 mL). The combined organic layers were washed with brine (3×10 mL), dried over anhydrous Na₂SO₄, and filtered. The filtrate was concentrated under reduced pressure and the resulting residue was purified by silica gel column chromatography to afford 5-bromo-3-{3-[(tert-butyldiphenylsilyl)oxy]-2,2-difluoropropyl}-1H-indole (2.1 g, 32% yield) as a yellow solid. ESI-MS m/z=530.1 [M+H+2]⁺; Calculated MW: 527.0.
Step 5. To a stirred solution of 5-bromo-3-{3-[(tert-butyldiphenylsilyl)oxy]-2,2-difluoropropyl}-1H-indole (1.5 g, 2.8 mmol, 1.0 equiv) and in THF (20 mL) were added AgOTf (875 mg, 3.4 mmol, 1.2 equiv) in portions at 0° C. under argon atmosphere. 12 (288 mg, 1.1 mmol, 0.4 equiv) was then added in portions at 0° C. The resulting mixture was stirred for an additional 10 min at 0° C. The reaction was quenched by the addition of sat. sodium hyposulfite (aq.) (20 mL) at 0° C. and extracted with EtOAc (3×20 mL). The combined organic layers were washed with brine (3×10 mL), dried over anhydrous Na₂SO₄, and filtered. The filtrate was concentrated under reduced pressure and the resulting residue was purified by silica gel column chromatography to afford 5-bromo-3-{3-[(tert-butyldiphenylsilyl)oxy]-2,2-difluoropropyl}-2-iodo-1H-indole (770 mg, 41% yield) as a yellow solid. ESI-MS m/z=652.2 [M−H]⁺; Calculated MW: 653.0.
Step 6. To a stirred solution of 5-bromo-3-{3-[(tert-butyldiphenylsilyl)oxy]-2,2-difluoropropyl}-2-iodo-1H-indole (670 mg, 1.0 mmol, 1.0 equiv) and 2-[(1S)-1-methoxyethyl]-3-(4,4,5,5-tetramethyl-1,3,2-dioxaborolan-2-yl)pyridine (404 mg, 1.5 mmol, 1.5 equiv) in dioxane (10 mL) and H₂O (2 mL) were added Pd(dppf)Cl₂ (74 mg, 0.1 mmol, 0.1 equiv) and K₂CO₃ (353 mg, 2.5 mmol, 2.5 equiv) in portions at room temperature under air atmosphere. The resulting mixture was stirred for 2 h at 80° C. under argon atmosphere. The mixture was then extracted with EtOAc (3×10 mL), and the combined organic layers were washed with brine (3×10 mL), dried over anhydrous Na₂SO₄, and filtered. The filtrate was concentrated under reduced pressure and the remaining residue was purified by silica gel column chromatography to afford 5-bromo-3-{3-[(tert-butyldiphenylsilyl)oxy]-2,2-difluoropropyl}-2-{2-[(1S)-1-methoxyethyl]pyridin-3-yl}-1H-indole (500 mg, 73% yield) as a yellow solid ESI-MS m/z=663.2 [M+H]⁺; Calculated MW: 662.2.
Step 7. To a stirred solution of 5-bromo-3-{3-[(tert-butyldiphenylsilyl)oxy]-2,2-difluoropropyl}-2-{2-[(1S)-1-methoxyethyl]pyridin-3-yl}-1H-indole (500 mg, 0.7 mmol, 1.0 equiv) in DMF (5 mL) was added Cs₂CO₃ (613 mg, 1.8 mmol, 2.5 equiv) and Iodoethane (152 mg, 0.97 mmol, 1.3 equiv) in portions at room temperature under air atmosphere. The resulting mixture was stirred for 2 h at room temperature under air atmosphere and the remaining residue was purified by chromatography to afford 5-bromo-3-{3-[(tert-butyldiphenylsilyl)oxy]-2,2-difluoropropyl}-1-ethyl-2-{2-[(1S)-1-methoxyethyl]pyridin-3-yl}indole (500 mg, 95% yield) as a yellow solid. ESI-MS m/z=693.4 [M+H]⁺; Calculated MW: 690.2.
Step 8. To a stirred solution of 5-bromo-3-{3-[(tert-butyldiphenylsilyl)oxy]-2,2-difluoropropyl}-1-ethyl-2-{2-[(1S)-1-methoxyethyl]pyridin-3-yl}indole (500 mg, 0.7 mmol, 1.0 equiv) in THF (5 mL) was added TBAF (3.6 mL, 3.6 mmol, 5 equiv) dropwise at room temperature under air atmosphere. The resulting mixture was stirred for overnight at 65° C. under air atmosphere. After which the mixture was extracted with EtOAc (3×5 mL) and the combined organic layers were washed with brine (3×5 mL), dried over anhydrous Na₂SO₄, and filtered. The filtrate was concentrated under reduced pressure and the resulting residue was purified by silica gel column chromatography to afford 3-[(2M)-5-bromo-1-ethyl-2-{2-[(1S)-1-methoxyethyl]pyridin-3-yl}indol-3-yl]-2,2-difluoropropan-1-ol (300 mg, 91% yield) as a yellow solid. ESI-MS m/z=455.1 [M+H+2]⁺; Calculated MW: 452.1.
Step 9. To a stirred mixture of 3-[(2M)-5-bromo-1-ethyl-2-{2-[(1S)-1-methoxyethyl]pyridin-3-yl}indol-3-yl]-2,2-difluoropropan-1-ol (300 mg, 0.6 mmol, 1.0 equiv) and methyl (3S)-1-[(2S)-2-[(tert-butoxycarbonyl) amino]-3-[3-(4,4,5,5-tetramethyl-1,3,2-dioxaborolan-2-yl)-5,6-dihydro-2H-pyridin-1-yl]propanoyl]-1,2-diazinane-3-carboxylate (414 mg, 0.79 mmol, 1.2 equiv) in dioxane (3 mL) and H₂O (0.6 mL) were added K₃PO₄ (351 mg, 1.6 mmol, 2.5 equiv) and Pd(dtbpf)Cl₂ (43 mg, 0.06 mmol, 0.1 equiv) in portions at room temperature under air atmosphere. The resulting mixture was stirred for 2 h at 80° C. under argon atmosphere after which the resulting mixture was extracted with EtOAc (3×10 mL). The combined organic layers were washed with brine (3×10 mL), dried over anhydrous Na₂SO₄, and filtered. The filtrate was concentrated under reduced pressure and the resulting residue was purified by chromatography to afford methyl (3S)-1-[(2S)-2-[(tert-butoxycarbonyl)amino]-3-{3-[(2M)-3-(2,2-difluoro-3-hydroxypropyl)-1-ethyl-2-{2-[(1S)-1-methoxyethyl]pyridin-3-yl}indol-5-yl]-5,6-dihydro-2H-pyridin-1-yl}propanoyl]-1,2-diazinane-3-carboxylate (400 mg, 78% yield) as a yellow solid. ESI-MS m/z=769.7 [M+H]⁺; Calculated MW: 768.4.
Step 10. To a stirred mixture of methyl (3S)-1-[(2S)-2-[(tert-butoxycarbonyl)amino]-3-{3-[3-(2,2-difluoro-3-hydroxypropyl)-1-ethyl-2-{2-[(1S)-1-methoxyethyl]pyridin-3-yl}indol-5-yl]-5,6-dihydro-2H-pyridin-1-yl}propanoyl]-1,2-diazinane-3-carboxylate (400 mg, 0.5 mmol, 1.0 equiv) in THF (4 mL) were added LiOH (62 mg, 2.6 mmol, 5 equiv) in H₂O (2.6 mL) in portions at 0° C. under air atmosphere. The resulting mixture was stirred for 2 h at room temperature under air atmosphere. The mixture was acidified to pH 6 with conc. HCl and the resulting mixture was extracted with EtOAc (3×10 mL). The combined organic layers were washed with brine (3×10 mL), dried over anhydrous Na₂SO₄, and filtered. The filtrate was concentrated under reduced pressure to afford (3S)-1-[(2S)-2-[(tert-butoxycarbonyl)amino]-3-{3-[3-(2,2-difluoro-3-hydroxypropyl)-1-ethyl-2-{2-[(1S)-1-methoxyethyl]pyridin-3-yl}indol-5-yl]-5,6-dihydro-2H-pyridin-1-yl}propanoyl]-1,2-diazinane-3-carboxylic acid (400 mg, crude) as a yellow solid. ESI-MS m/z=755.4 [M+H]⁺; Calculated MW: 754.4.
Step 11. To a stirred mixture of (3S)-1-[(2S)-2-[(tert-butoxycarbonyl)amino]-3-{3-[3-(2,2-difluoro-3-hydroxypropyl)-1-ethyl-2-{2-[(1S)-1-methoxyethyl]pyridin-3-yl}indol-5-yl]-5,6-dihydro-2H-pyridin-1-yl}propanoyl]-1,2-diazinane-3-carboxylic acid (440 mg, 0.6 mmol, 1.0 equiv) in DCM (44 mL) were added DIEA (2.2 g, 17.4 mmol, 30 equiv), HOBT (393 mg, 2.9 mmol, 5 equiv) and EDCl (2.2 g, 11.6 mmol, 20 equiv) in portions at 0° C. under air atmosphere. The resulting mixture was stirred overnight at room temperature under air atmosphere after which the mixture was concentrated under reduced pressure. The resulting residue was extracted with EtOAc (3×30 mL), and the combined organic layers were washed with brine (3×30 mL), dried over anhydrous Na₂SO₄, and filtered. The filtrate was concentrated under reduced pressure and the resulting residue was purified by silica gel column chromatography to afford the mixture of atropisomers tert-butyl ((6³S,4S)-1¹-ethyl-10,10-difluoro-1²-(2-((S)-1-methoxyethyl)pyridin-3-yl)-5,7-dioxo-2¹,2²,2³,2⁶,6¹,6²,6³,6⁴,6⁵,6⁶-decahydro-1¹H-8-oxa-1(5,3)-indola-6(1,3)-pyridazina-2(5,1)- pyridinacycloundecaphane-4-yl)carbamate (270 mg, 63% yield) as a yellow solid. ESI-MS m/z=737.6 [M+H]⁺; Calculated MW: 736.4. The atropisomer mixture was purified by chiral HPLC to afford isomer 1 (80 mg) as a yellow solid and isomer 2 (80 mg) as a yellow solid.

### Intermediate 15. Synthesis of tert-butyl ((6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-piperidinacycloundecaphane-4-yl)carbamate

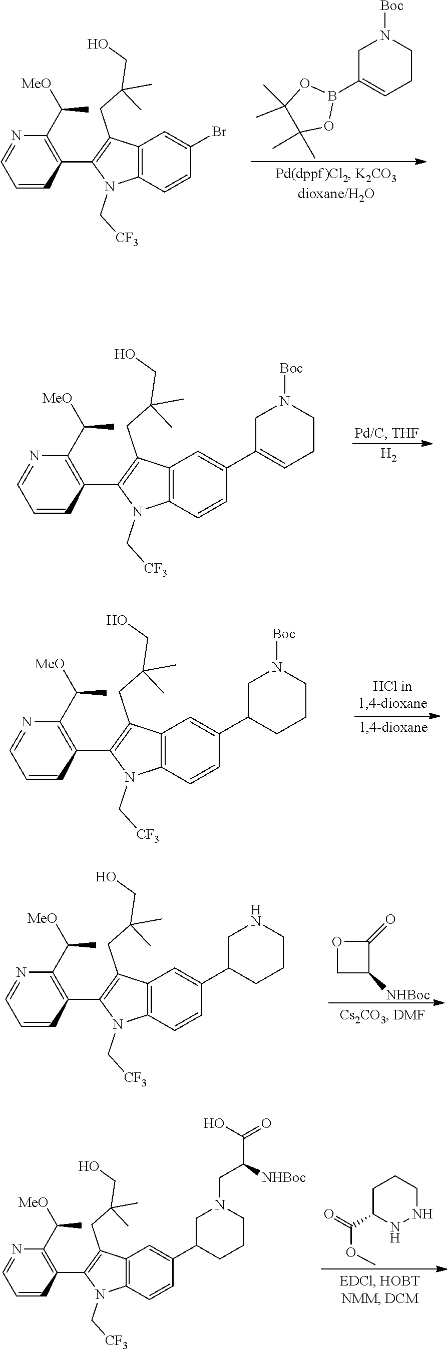

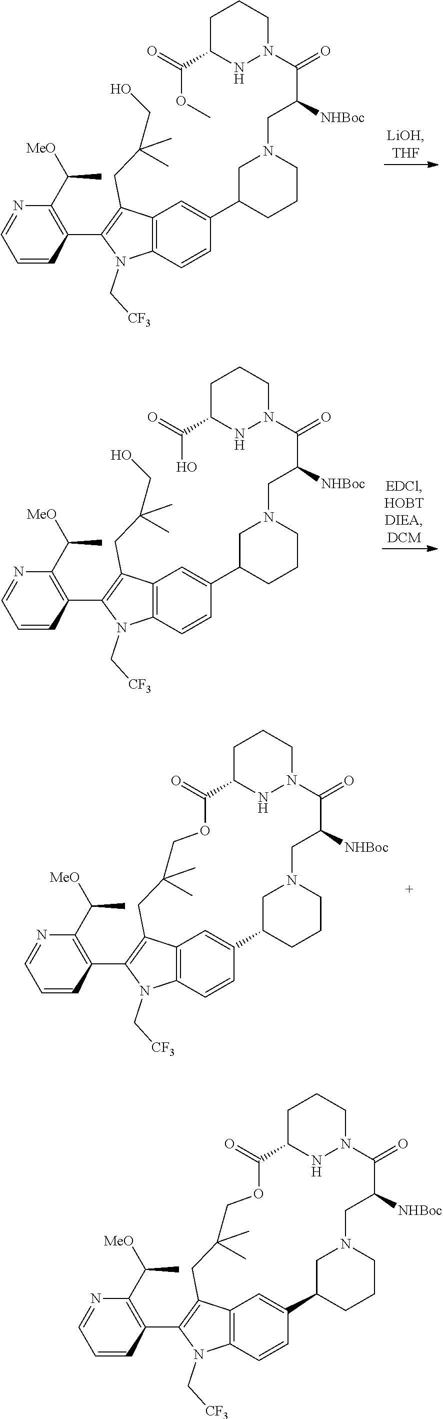

Step 1. To a mixture of 3-[(2M)-5-bromo-2-[2-[(1S)-1-methoxyethyl]pyridin-3-yl]-1-(2,2,2-trifluoroethyl)indol-3-yl]-2,2-dimethylpropan-1-ol (10.0 g, 20.0 mmol) and tert-butyl 3-(4,4,5,5-tetramethyl-1,3,2-dioxaborolan-2-yl)-5,6-dihydro-2H-pyridine-1-carboxylate (9.29 g, 30.0 mmol) in 1,4-dioxane (85 mL) and H₂O (17 mL) under an atmosphere of N₂ was added Pd(dppf)Cl₂ (0.73 g, 1.0 mmol) and K₂CO₃ (6.92 g, 50.1 mmol) in portions. The mixture was heated to 80° C. and stirred for 3 h, then the mixture extracted with EtOAc (3×100 mL). The combined organic layers were washed with brine (3×100 mL), dried over anhydrous Na₂SO₄, and filtered. The filtrate was concentrated under reduced pressure and the residue was purified by silica gel column chromatography to give tert-butyl 3-[(2M)-3-(3-hydroxy-2,2-dimethylpropyl)-2-[2-[(1S)-1-methoxyethyl]pyridin-3-yl]-1-(2,2,2-trifluoroethyl)indol-5-yl]-5,6-dihydro-2H-pyridine-1-carboxylate (9.0 g, 67% yield) as a solid. LCMS (ESI): m/z [M+H]⁺ calc'd for C₃₃H₄₂F₃N₃O₄ 601.3; found 602.3.
Step 2. A mixture of tert-butyl 3-[(2M)-3-(3-hydroxy-2,2-dimethylpropyl)-2-[2-[(1S)-1-methoxyethyl]pyridin-3-yl]-1-(2,2,2-trifluoroethyl)indol-5-yl]-5,6-dihydro-2H-pyridine-1-carboxylate (6.00 g, 10.0 mmol) and Pd/C (605 mg, 5.7 mmol) in THF (60 mL) was stirred under an atmosphere of H₂ overnight. The mixture was filtered, and the filtrate was concentrated under reduced pressure to give tert-butyl 3-[(2M)-3-(3-hydroxy-2,2-dimethylpropyl)-2-[2-[(1S)-1-methoxyethyl]pyridin-3-yl]-1-(2,2,2-trifluoroethyl)indol-5-yl]piperidine-1-carboxylate (5.8 g) as an oil. LCMS (ESI): m/z [M+H]⁺ calc'd for C₃₃H₄₄F₃N₃O₄ 603.3; found 604.3.
Step 3. To a mixture of tert-butyl 3-[(2M)-3-(3-hydroxy-2,2-dimethylpropyl)-2-[2-[(1S)-1-methoxyethyl]pyridin-3-yl]-1-(2,2,2-trifluoroethyl)indol-5-yl]piperidine-1-carboxylate (5.70 g, 9.4 mmol) in 1,4-dioxane (30 mL) at 0° C. under an atmosphere of N₂ was added HCl in 1,4-dioxane (30 mL). The mixture was stirred at 0° C. for 2 h, then aqueous NaHCO₃ was added, and the mixture was extracted with EtOAc (3×20 mL). The combined organic layers were washed with brine (3×20 mL), dried over anhydrous Na₂SO₄, filtered and the filtrate was concentrated under reduced pressure to give 3-[(2M)-2-[2-[(1S)-1-methoxyethyl]pyridin-3-yl]-5-(piperidin-3-yl)-1-(2,2,2-trifluoroethyl)indol-3-yl]-2,2-dimethylpropan-1-ol (4.8 g) as an oil. LCMS (ESI): m/z [M+H]⁺ calc'd for C₂₈H₃₆F₃N₃O₂ 503.3; found 504.3.
Step 4. To a mixture of 3-[(2M)-2-[2-[(1S)-1-methoxyethyl]pyridin-3-yl]-5-(piperidin-3-yl)-1-(2,2,2-trifluoroethyl)indol-3-yl]-2,2-dimethylpropan-1-ol (4.6 g, 9.1 mmol) in DMF (46 mL) under an atmosphere of N₂ was added tert-butyl N-[(3S)-2-oxooxetan-3-yl]carbamate (3.46 g, 18.3 mmol) and Cs₂CO₃ (7.44 g, 22.8 mmol). The mixture was heated to 40° C. and stirred for 2 h, then H₂O was added, and the mixture was extracted with EtOAc (3×50 mL). The combined organic layers were washed with brine (3×50 mL), dried over anhydrous Na₂SO₄, and filtered. The filtrate was concentrated under reduced pressure and the residue was purified by preparative-HPLC to give (2S)-2-[(tert-butoxycarbonyl)amino]-3-[3-[(2M)-3-(3-hydroxy-2,2-dimethylpropyl)-2-[2-[(1S)-1-methoxyethyl]pyridin-3-yl]-1-(2,2,2-trifluoroethyl)indol-5-yl]piperidin-1-yl]propanoic acid (2.7 g, 39% yield) as a solid. LCMS (ESI): m/z [M+H]⁺ calc'd for C₃₆H₄₉F₃N₄O₆ 690.4; found 691.1.
Step 5. To a mixture of methyl (3S)-1,2-diazinane-3-carboxylate (835 mg, 5.79 mmol) in DCM (20 mL) at 0° C. under an atmosphere of N₂ was added NMM (2.93 g, 29.0 mmol), (2S)-2-[(tert-butoxycarbonyl)amino]-3-[3-[(2M)-3-(3-hydroxy-2,2-dimethylpropyl)-2-[2-[(1S)-1-methoxyethyl]pyridin-3-yl]-1-(2,2,2-trifluoroethyl)indol-5-yl]piperidin-1-yl]propanoic acid (2.00 g, 2.9 mmol), EDCl (833 mg, 4.3 mmol) and HOBT (196 mg, 1.5 mmol) in portions. The mixture was stirred at rt for h, then H₂O was added, and the mixture extracted with DCM (3×10 mL). The combined organic layers were washed with brine (3×10 mL), dried over anhydrous Na₂SO₄, and filtered. The filtrate was concentrated under reduced pressure and the residue was purified by silica gel column chromatography to give methyl (3S)-1-[(2S)-2-[(tert-butoxycarbonyl)amino]-3-[3-[(2M)-3-(3-hydroxy-2,2-dimethylpropyl)-2-[2-[(1S)-1-methoxyethyl]pyridin-3-yl]-1-(2,2,2-trifluoroethyl)indol-5-yl]piperidin-1-yl]propanoyl]-1,2-diazinane-3-carboxylate (2.0 g, 72% yield) as a solid. LCMS (ESI): m/z [M+H]⁺ calc'd for C₄₂H₅₉F₃N₆O₇ 816.4; found 817.5.
Step 6. To a mixture of methyl (3S)-1-[(2S)-2-[(tert-butoxycarbonyl)amino]-3-[3-[(2M)-3-(3-hydroxy-2,2-dimethylpropyl)-2-[2-[(1S)-1-methoxyethyl]pyridin-3-yl]-1-(2,2,2-trifluoroethyl)indol-5-yl]piperidin-1-yl]propanoyl]-1,2-diazinane-3-carboxylate (2.0 g, 2.5 mmol) in THF (20 mL) under an atmosphere of N₂ was added 1M LiOH (12.24 mL, 12.24 mmol). The mixture was stirred at rt, then acidified to pH ˜6 with 1M HCl and the mixture extracted with EtOAc (3×10 mL). The combined organic layers were washed with brine (3×10 mL), dried over anhydrous Na₂SO₄, filtered and the filtrate was concentrated under reduced pressure to give (3S)-1-[(2S)-2-[(tert-butoxycarbonyl)amino]-3-[3-[(2M)-3-(3-hydroxy-2,2-dimethylpropyl)-2-[2-[(1S)-1-methoxyethyl]pyridin-3-yl]-1-(2,2,2-trifluoroethyl)indol-5-yl]piperidin-1-yl]propanoyl]-1,2-diazinane-3-carboxylic acid (1.8 g) as a solid. LCMS (ESI): m/z [M+H]⁺ calc'd for C₄₁H₅₇F₃N₆O₇ 802.4; found 803.5.
Step 7. To a mixture of (3S)-1-[(2S)-2-[(tert-butoxycarbonyl)amino]-3-[3-[(2M)-3-(3-hydroxy-2,2-dimethylpropyl)-2-[2-[(1S)-1-methoxyethyl]pyridin-3-yl]-1-(2,2,2-trifluoroethyl)indol-5-yl]piperidin-1-yl]propanoyl]-1,2-diazinane-3-carboxylic acid (1.80 g, 2.2 mmol) in DCM (360 mL) at 0° C. under an atmosphere of N₂ was added DIPEA (8.69 g, 67.3 mmol), HOBT (1.51 g, 11.2 mmol) and EDCl (8.60 g, 44.8 mmol) in portions. The mixture was stirred at rt for h, H₂O was added, and the mixture was extracted with DCM (3×10 mL) The combined organic layers were washed with brine (3×10 mL), dried over anhydrous Na₂SO₄, and filtered. The filtrate was concentrated under reduced pressure and the residue was purified by preparative-TLC to give two diastereomers of tert-butyl ((6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)- pyridazina-2(3,1)-piperidinacycloundecaphane-4-yl)carbamate (160 mg, 9% yield) and (140 mg, 8% yield) both as solid. LCMS (ESI): m/z [M+H]⁺ calc'd for C₄₁H₅₅F₃N₆O₆ 784.4; found 785.7.

### Intermediate 16. Synthesis of benzyl ((6³S,4S,Z)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(4,2)-oxazola-1(5,3)-indola-6(1,3)-pyridazinacycloundecaphane-4-yl) carbamate

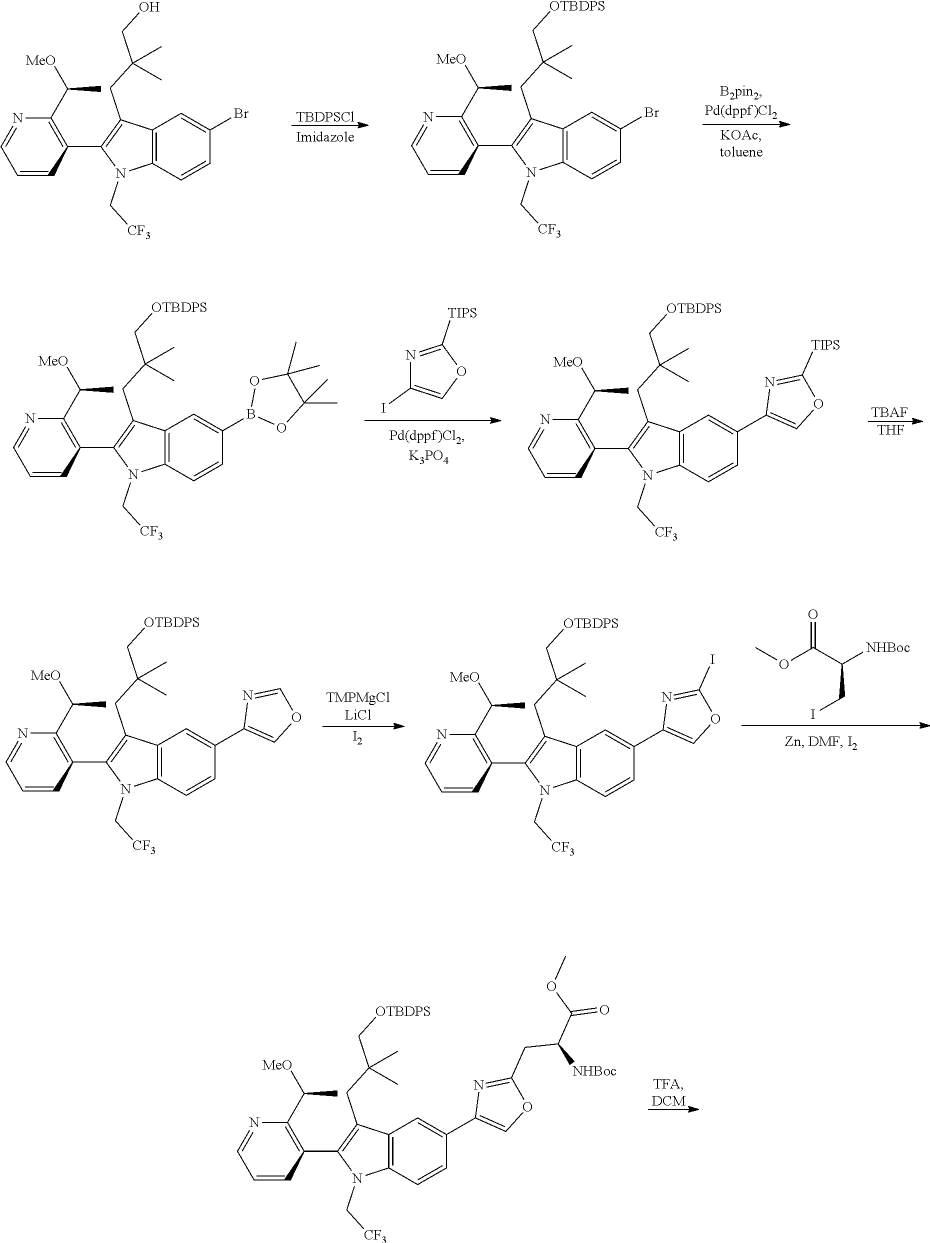

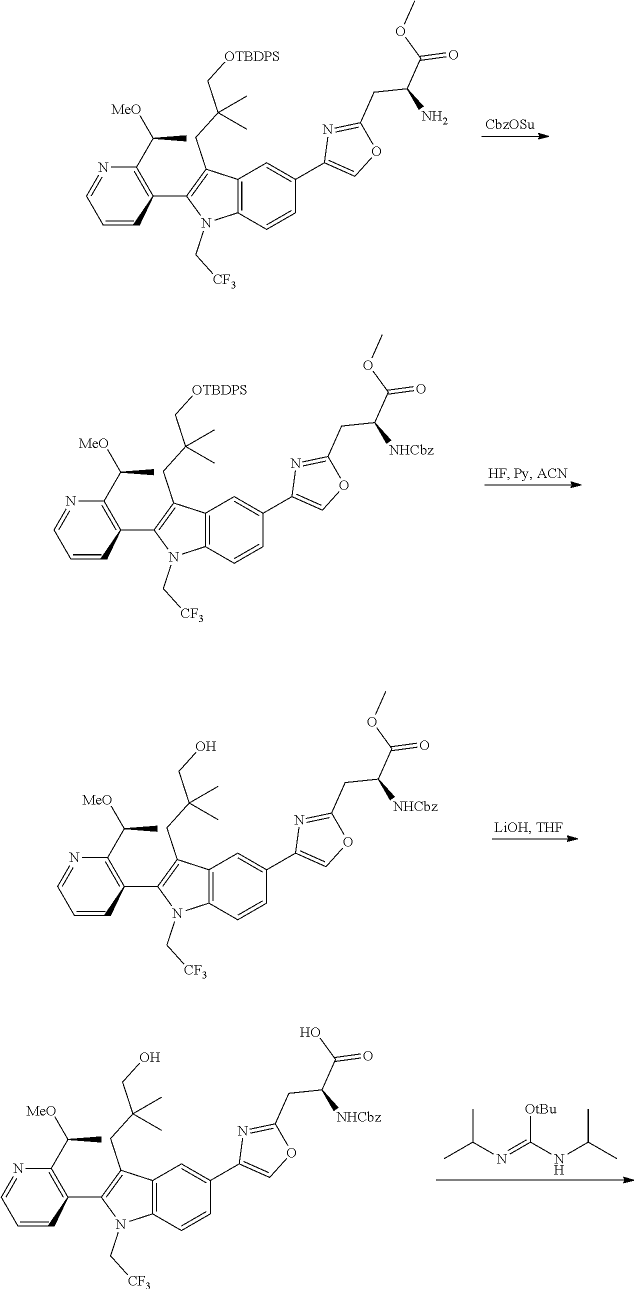

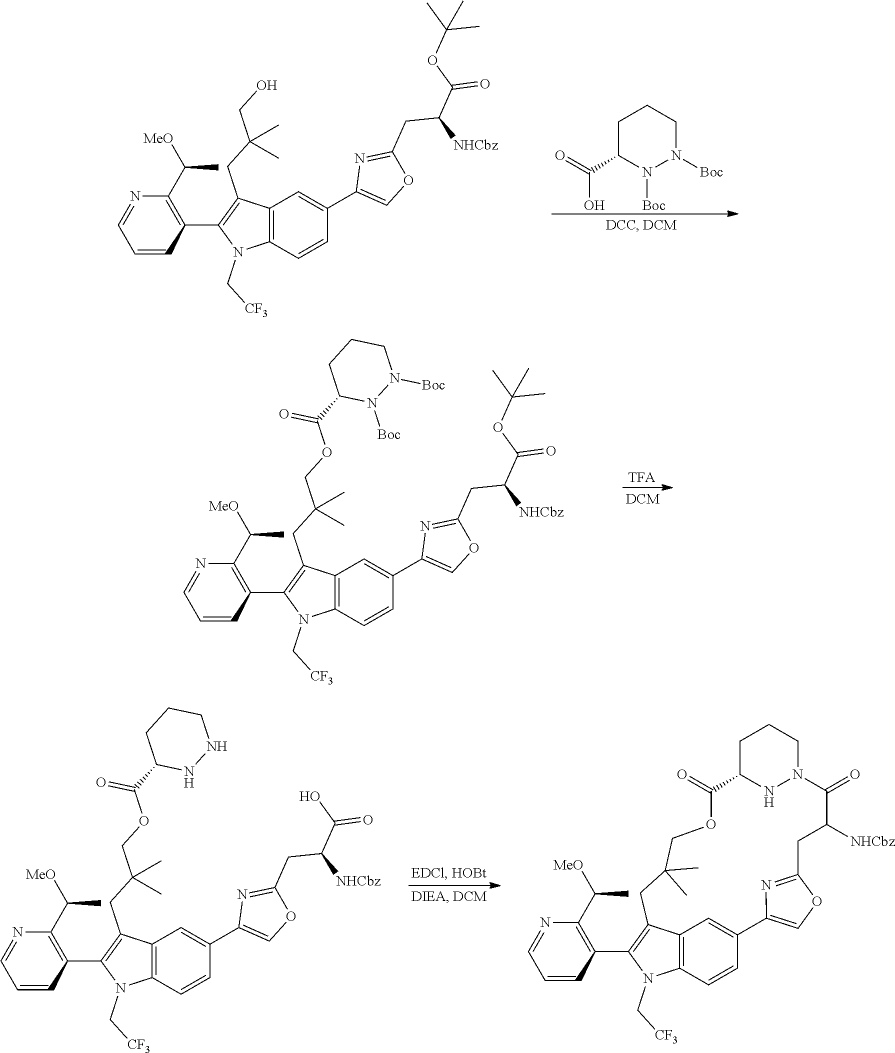

Step 1. To a mixture of (S)-3-(3-((tert-butyldiphenylsilyl)oxy)-2,2-dimethylpropyl)-2-(2-(1-methoxyethyl)pyridin-3-yl)-5-(4,4,5,5-tetramethyl-1,3,2-dioxaborolan-2-yl)-1-(2,2,2-trifluoroethyl)-1H-indole (6.3 g, 8.0 mmol) and 4-iodo-2-(triisopropylsilyl)-1,3-oxazole (8.46 g, 24.1 mmol) in 1,4-dioxane (60 mL) and H₂O (12 mL) under an atmosphere of Ar was added K₃PO₄ (4.26 g, 20.1 mmol) and Pd(dppf)Cl₂ (0.59 g, 0.80 mmol). The mixture was heated to 70° C. and stirred for 2 h, then concentrated under reduced pressure and the residue was purified by silica gel column chromatography to give (S)-4-(3-(3-((tert-butyldiphenylsilyl)oxy)-2,2-dimethylpropyl)-2-(2-(1-methoxyethyl)pyridin-3-yl)-1-(2,2,2-trifluoroethyl)-1H-indol-5-yl)-2-(triisopropylsilyl)oxazole (8.84 g) as an oil. LCMS (ESI): m/z [M+H]⁺ calc'd for C₅₁H₆₆F₃N₃O₃Si₂ 881.5; found 882.5.
Step 2. To a mixture of (2M)-3-[3-[(tert-butyldiphenylsilyl)oxy]-2,2-dimethylpropyl]-2-[2-[(1S)-1-methoxyethyl]pyridin-3-yl]-1-(2,2,2-trifluoroethyl)-5-[2-(triisopropylsilyl)-1,3-oxazol-4-yl]indole (8.84 g, 10.0 mmol) in THF (90 mL) at 0° C. was added 1M TBAF in THF (10.0 mL, 10.0 mmol). The mixture was stirred at 0° C. for 1 h, then washed with saturated NH₄Cl (3×100 mL). The combined aqueous layers were extracted with EtOAc (3×100 mL), and the combined organic layers were dried over anhydrous Na₂SO₄ and filtered. The filtrate was concentrated under reduced pressure to give (2M)-3-[3-[(tert-butyldiphenylsilyl)oxy]-2,2-dimethylpropyl]-2-[2-[(1S)-1-methoxyethyl]pyridin-3-yl]-5-(1,3-oxazol-4-yl)-1-(2,2,2-trifluoroethyl)indole (8.4 g) as an oil. LCMS (ESI): m/z [M+H]⁺ calc'd for C₄₂H₄₆F₃N₃O₃Si 725.3; found 726.4.
Step 3. To a mixture of (2M)-3-[3-[(tert-butyldiphenylsilyl)oxy]-2,2-dimethylpropyl]-2-[2-[(1S)-1-methoxyethyl]pyridin-3-yl]-5-(1,3-oxazol-4-yl)-1-(2,2,2-trifluoroethyl)indole (4.5 g, 6.2 mmol) in THF (45 mL) at 0° C. under an atmosphere of N₂ was added 1M TMPMgCl·LiCl (12.2 mL, 12.2 mmol) dropwise. The mixture was warmed to rt and stirred for 1 h, then a mixture of 12 (1.89 g, 7.4 mmol) in THF (10 mL) was added dropwise. The mixture was stirred at rt for 1 h, then re-cooled to 0° C. and saturated NH₄Cl added, and the mixture extracted with EtOAc (3×10 mL). The combined organic layers were washed with brine (2×10 mL), dried over anhydrous Na₂SO₄, and filtered. The filtrate was concentrated under reduced pressure and the residue was purified by preparative-TLC to give (S)-4-(3-(3-((tert-butyldiphenylsilyl)oxy)-2,2-dimethylpropyl)-2-(2-(1-methoxyethyl)pyridin-3-yl)-1-(2,2,2-trifluoroethyl)-1H-indol-5-yl)-2-iodooxazole (3.0 g, 57% yield) as a solid. LCMS (ESI): m/z [M+H]⁺ calc'd for C₄₂H₄₅F₃IN₃O₃Si 851.2; found 852.3.
Step 4. To a mixture of Zn (645 mg, 9.9 mmol) in DMF (10 mL) under an atmosphere of Ar was added 12 (125 mg, 0.49 mmol). The mixture was heated to 45° C. and stirred for 30 min, then methyl (2R)-2-[(tert-butoxycarbonyl)amino]-3-iodopropanoate (1.22 g, 3.7 mmol) in DMF (5 mL) was added dropwise at 45° C. The mixture was stirred at 45° C. for 2 h then cooled to 0° C. and(S)-4-(3-(3-((tert-butyldiphenylsilyl)oxy)-2,2-dimethylpropyl)-2-(2-(1-methoxyethyl)pyridin-3-yl)-1-(2,2,2-trifluoroethyl)-1H-indol-5-yl)-2-iodooxazole (2.1 g, 2.5 mmol), then Pd(PPh₃)₂Cl₂ (173 mg, 0.25 mmol) in DMF (20 mL) added dropwise. The mixture was heated to 75° C. and stirred for 2 h, then brine (20 mL) added, and the mixture extracted with EtOAc (3×50 mL). The combined organic layers were washed with brine (3×10 mL), dried over anhydrous Na₂SO₄, and filtered. The filtrate was concentrated under reduced pressure and the residue was purified by silica gel column chromatography to give methyl (S)-2-((tert-butoxycarbonyl)amino)-3-(4-(3-(3-((tert-butyldiphenylsilyl)oxy)-2,2-dimethylpropyl)-2-(2-((S)-1-methoxyethyl)pyridin-3-yl)-1-(2,2,2-trifluoroethyl)-1H-indol-5-yl)oxazol-2-yl)propanoate (1.6 g, 70% yield) as an oil. LCMS (ESI): m/z [M+H]⁺ calc'd for C₅₁H₆₁F₃N₄O₇Si 926.4; found 927.5.
Step 5. To a mixture of (S)-2-((tert-butoxycarbonyl)amino)-3-(4-(3-(3-((tert-butyldiphenylsilyl)oxy)-2,2-dimethylpropyl)-2-(2-((S)-1-methoxyethyl)pyridin-3-yl)-1-(2,2,2-trifluoroethyl)-1H-indol-5-yl)oxazol-2-yl)propanoate (2.4 g, 2.6 mmol) in DCM (1.8 mL) at 0° C. was added TFA (0.6 mL). The mixture was stirred at 0° C. for 1 h, then saturated NaHCO₃ was added, and the mixture was extracted with DCM/MeOH (10:1; 3×50 mL). The combined organic layers were washed with brine (3×20 mL), dried over anhydrous Na₂SO₄, filtered and the filtrate was concentrated under reduced pressure to give methyl (S)-2-amino-3-(4-(3-(3-((tert-butyldiphenylsilyl)oxy)-2,2-dimethylpropyl)-2-(2-((S)-1-methoxyethyl)pyridin-3-yl)-1-(2,2,2-trifluoroethyl)-1H-indol-5-yl)oxazol-2-yl)propanoate (2.1 g, 98% yield) as a solid. LCMS (ESI): m/z [M+H]⁺ calc'd for C₄₆H₅₃F₃N₄O₅Si 826.4; found 827.5.
Step 6. To a mixture of (S)-2-amino-3-(4-(3-(3-((tert-butyldiphenylsilyl)oxy)-2,2-dimethylpropyl)-2-(2-((S)-1-methoxyethyl)pyridin-3-yl)-1-(2,2,2-trifluoroethyl)-1H-indol-5-yl)oxazol-2-yl)propanoate (2.1 g, 2.5 mmol) in THF (15 mL) and H₂O (5 mL) at 0° C. was added NaHCO₃ (0.64 g, 7.6 mmol) and benzyl 2,5-dioxopyrrolidin-1-yl carbonate (0.95 g, 3.8 mmol). The mixture was stirred at 0° C. for 1 h then EtOAc (20 mL) added, and the mixture was washed with brine (3×10 mL). The organic layer was concentrated under reduced pressure and the residue was purified by silica gel column chromatography to give methyl (S)-2-(((benzyloxy)carbonyl)amino)-3-(4-(3-(3-((tert-butyldiphenylsilyl)oxy)-2,2-dimethylpropyl)-2-(2-((S)-1-methoxyethyl)pyridin-3-yl)-1-(2,2,2-trifluoroethyl)-1H-indol-5-yl)oxazol-2-yl)propanoate (2.2 g, 90% yield) as a solid. LCMS (ESI): m/z [M+H]⁺ calc'd for C₅₄H₅₉F₃N₄O₇Si 960.4; found 961.4.
Step 7. To a mixture of methyl (S)-2-(((benzyloxy)carbonyl)amino)-3-(4-(3-(3-((tert-butyldiphenylsilyl)oxy)-2,2-dimethylpropyl)-2-(2-((S)-1-methoxyethyl)pyridin-3-yl)-1-(2,2,2-trifluoroethyl)-1H-indol-5-yl)oxazol-2-yl)propanoate (2.2 g, 2.3 mmol) in ACN (11 mL) at 0° C. was added HF-pyridine (11 mL, 122 mmol). The mixture was warmed to rt and stirred for 1 h, then basified to PH ˜7 with saturated NaHCO₃. The aqueous and organic layers were partitioned and the organic layer was concentrated under reduced pressure to give methyl (S)-2-(((benzyloxy)carbonyl)amino)-3-(4-(3-(3-hydroxy-2,2-dimethylpropyl)-2-(2-((S)-1-methoxyethyl)pyridin-3-yl)-1-(2,2,2-trifluoroethyl)-1H-indol-5-yl)oxazol-2-yl)propanoate (1.7 g) as a solid. LCMS (ESI): m/z [M+H]⁺ calc'd for C₃₈H₄₁F3N₄O₇ 722.3; found 723.3.
Step 8. To a mixture of methyl (S)-2-(((benzyloxy)carbonyl)amino)-3-(4-(3-(3-hydroxy-2,2-dimethylpropyl)-2-(2-((S)-1-methoxyethyl)pyridin-3-yl)-1-(2,2,2-trifluoroethyl)-1H-indol-5-yl)oxazol-2-yl)propanoate (1.7 g, 2.4 mmol) in THF (1.2 mL) and H₂O (0.4 mL) at 0° C. was added LiOH (0.08 g, 3.5 mmol). The mixture was stirred at 0° C. overnight, then acidified to PH ˜4 with aqueous HCl. The mixture was extracted with DCM/MeOH (10:1; 3×20 mL). The combined organic layers were dried over anhydrous Na₂SO₄, filtered and the filtrate was concentrated under reduced pressure to give (2S)-2-{[(benzyloxy)carbonyl]amino}-3-{4-[(2M)-3-(3-hydroxy-2,2-dimethylpropyl)-2-{2-[(1S)-1-methoxyethyl]pyridin-3-yl}-1-(2,2,2-trifluoroethyl)indol-5-yl]-1,3-oxazol-2-yl}propanoic acid (1.5 g, 90% yield) as an oil. LCMS (ESI): m/z [M+H]⁺ calc'd for C₃₇H₃₉F₃N₄O₇ 708.3; found 709.3.
Step 9. To a mixture of (2S)-2-{[(benzyloxy)carbonyl]amino}-3-{4-[(2M)-3-(3-hydroxy-2,2-dimethylpropyl)-2-{2-[(1S)-1-methoxyethyl]pyridin-3-yl}-1-(2,2,2-trifluoroethyl)indol-5-yl]-1,3-oxazol-2-yl}propanoic acid (1.5 g, 2.1 mmol) in DCM (15 mL) and (Z)—N,N′-diisopropyl tert-butoxymethanimidamide (2.12 mL, 10.6 mmol). The mixture was heated to 40° C. and stirred for 3 h, then concentrated under reduced pressure and the residue was purified by silica gel column chromatography to give tert-butyl(S)-2-(((benzyloxy)carbonyl)amino)-3-(4-(3-(3-hydroxy-2,2-dimethylpropyl)-2-(2-((S)-1-methoxyethyl)pyridin-3-yl)-1-(2,2,2-trifluoroethyl)-1H-indol-5-yl)oxazol-2-yl)propanoate (1.6 g, 99% yield) as a solid. LCMS (ESI): m/z [M+H]⁺ calc'd for C₄₁H₄₇F₃N₄O₇ 764.3; found 765.3.
Step 10. To a mixture of tert-butyl(S)-2-(((benzyloxy)carbonyl)amino)-3-(4-(3-(3-hydroxy-2,2-dimethylpropyl)-2-(2-((S)-1-methoxyethyl)pyridin-3-yl)-1-(2,2,2-trifluoroethyl)-1H-indol-5-yl)oxazol-2-yl)propanoate (1.8 g, 2.4 mmol) in DCM (16 mL) at 0° C. was added (3S)-1,2-bis (tert-butoxycarbonyl)-1,2-diazinane-3-carboxylic acid (1.04 g, 3.1 mmol) and DCC (0.65 g, 3.1 mmol). The mixture was stirred at 0° C. for 1 h, then concentrated under reduced pressure and the residue was purified by silica gel column chromatography to give 3-(3-(5-(2-((S)-2-(((benzyloxy)carbonyl)amino)-3-(tert-butoxy)-3-oxopropyl)oxazol-4-yl)-2-(2-((S)-1-methoxyethyl)pyridin-3-yl)-1-(2,2,2-trifluoroethyl)-1H-indol-3-yl)-2,2-dimethylpropyl) 1,2-di-tert-butyl(S)-tetrahydropyridazine-1,2,3-tricarboxylate (1.8 g, 80% yield) as a solid. LCMS (ESI): m/z [M+H]⁺ calc'd for C₅₆H₇₁F₃N₆O₁₂ 1076.5; found 1077.4.
Step 11. To a mixture of 3-(3-(5-(2-((S)-2-(((benzyloxy)carbonyl)amino)-3-(tert-butoxy)-3-oxopropyl)oxazol-4-yl)-2-(2-((S)-1-methoxyethyl)pyridin-3-yl)-1-(2,2,2-trifluoroethyl)-1H-indol-3-yl)-2,2-dimethylpropyl) 1,2-di-tert-butyl(S)-tetrahydropyridazine-1,2,3-tricarboxylate (1.8 g, 1.7 mmol) in DCM (15 mL) at 0° C. was added TFA (5 mL). The mixture was stirred at 0° C. for 1 h, then saturated NaHCO₃ was added, and the mixture extracted with EtOAc (3×100 mL). The combined organic layers were washed with brine (3×10 mL), dried over anhydrous Na₂SO₄, filtered and the filtrate was concentrated under reduced pressure to give (S)-2-(((benzyloxy)carbonyl)amino)-3-(4-(3-(3-(((S)-hexahydropyridazine-3-carbonyl)oxy)-2,2-dimethylpropyl)-2-(2-((S)-1-methoxyethyl)pyridin-3-yl)-1-(2,2,2-trifluoroethyl)-1H-indol-5-yl)oxazol-2-yl)propanoic acid (1.27 g, 93% yield) as a solid. LCMS (ESI): m/z [M+H]⁺ calc'd for C₄₂H₄₇F₃N₆O₈ 820.3; found 821.4.
Step 12. To a mixture of (S)-2-(((benzyloxy)carbonyl)amino)-3-(4-(3-(3-(((S)-hexahydropyridazine-3-carbonyl)oxy)-2,2-dimethylpropyl)-2-(2-((S)-1-methoxyethyl)pyridin-3-yl)-1-(2,2,2-trifluoroethyl)-1H-indol-5-yl)oxazol-2-yl)propanoic acid (870 mg, 1.1 mmol) and DIPEA (4.1 g, 31.8 mmol) in DCM (175 mL) at 0° C. was added HOBt (1.15 g, 8.5 mmol) and EDCl (8.13 g, 42.4 mmol) in portions over 15 min. The mixture was allowed to warm to rt and stirred overnight then concentrated under reduced pressure and the residue was purified by silica gel column chromatography to give benzyl ((6³S,4S,Z)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(4,2)-oxazola-1(5,3)-indola-6(1,3)-pyridazinacycloundecaphane-4-yl)carbamate (180 mg, 21% yield) as a solid. LCMS (ESI): m/z [M+H]⁺ calc'd for C₄₂H₄₅F₃N₆O₇ 802.3; found 803.4.

### Intermediate 17. Synthesis of tert-butyl ((6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-pyrrolidinacycloundecaphane-4-yl)carbamate

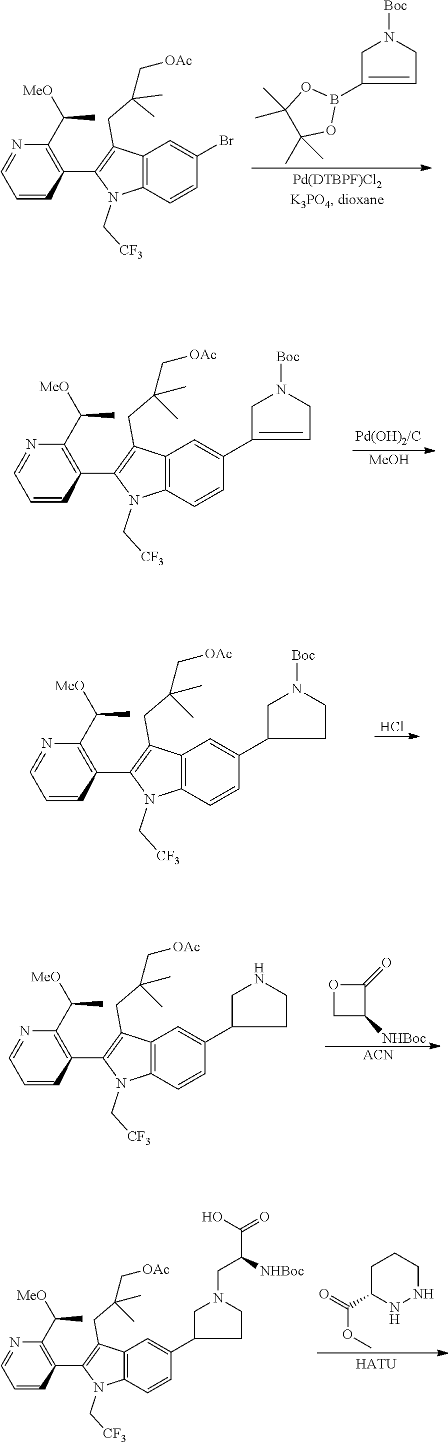

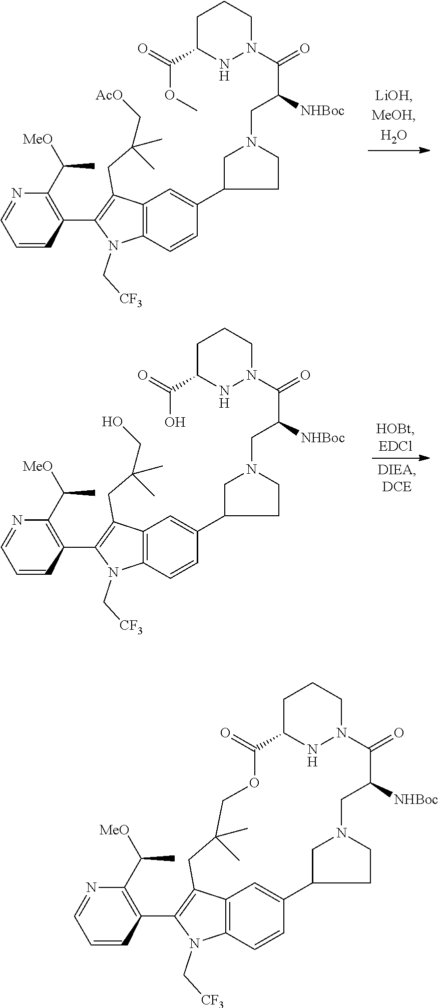

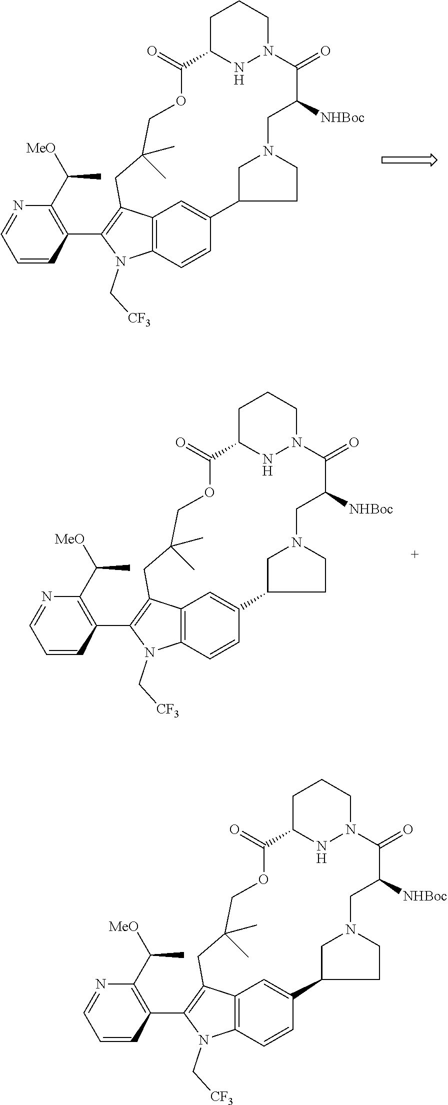

Step 1. To a mixture of (S)-3-(5-bromo-2-(2-(1-methoxyethyl)pyridin-3-yl)-1-(2,2,2-trifluoroethyl)-1H-indol-3-yl)-2,2-dimethylpropyl acetate (10 g, 18.5 mmol) and tert-butyl 3-(4,4,5,5-tetramethyl-1,3,2-dioxaborolan-2-yl)-2,5-dihydro-1H-pyrrole-1-carboxylate (8.18 g, 27.7 mmol) in dioxane (100 mL) and H₂O (20 mL) under an atmosphere of Ar was added Pd(DTBPF)Cl₂ (1.20 g, 1.85 mmol) and K₃PO₄ (9.80 g, 46.2 mmol). The mixture was heated to 85° C. and stirred for 1 h, then extracted with EtOAc (10 mL). The combined organic layers were washed with brine (8×5 mL), dried over anhydrous Na₂SO₄, and filtered. The filtrate was concentrated under reduced pressure and the residue was purified by silica gel column chromatography to give tert-butyl(S)-3-(3-(3-acetoxy-2,2-dimethylpropyl)-2-(2-(1-methoxyethyl)pyridin-3-yl)-1-(2,2,2-trifluoroethyl)-1H-indol-5-yl)-2,5-dihydro-1H-pyrrole-1-carboxylate (13 g, 89% yield) as an oil. LCMS (ESI): m/z [M+H]⁺ calc'd for C₃₄H₄₂F₃N₃O₅ 629.3; found 630.4.
Step 2. A mixture of tert-butyl(S)-3-(3-(3-acetoxy-2,2-dimethylpropyl)-2-(2-(1-methoxyethyl)pyridin-3-yl)-1-(2,2,2-trifluoroethyl)-1H-indol-5-yl)-2,5-dihydro-1H-pyrrole-1-carboxylate (10.75 g, 17.1 mmol) and Pd(OH)₂/C (3.2 g, 22.8 mmol) in MeOH (100 mL) was heated to 40° C. and under an atmosphere of H₂ for 2 h. The mixture was filtered, and the filter cake was washed with DCM (10×10 mL). The filtrate was concentrated under reduced pressure and the residue was purified by preparative-HPLC to give tert-butyl 3-(3-(3-acetoxy-2,2-dimethylpropyl)-2-(2-((S)-1-methoxyethyl)pyridin-3-yl)-1-(2,2,2-trifluoroethyl)-1H-indol-5-yl)pyrrolidine-1-carboxylate (6.4 g, 56% yield) as an oil. LCMS (ESI): m/z [M+H]⁺ calc'd for C₃₄H₄₄F₃N₃O₅ 631.3; found 632.4.
Step 3. To a mixture of tert-butyl 3-(3-(3-acetoxy-2,2-dimethylpropyl)-2-(2-((S)-1-methoxyethyl)pyridin-3-yl)-1-(2,2,2-trifluoroethyl)-1H-indol-5-yl)pyrrolidine-1-carboxylate (7.0 g, 11.1 mmol) in dioxane (70 mL) was added HCl in 1,4-dioxane (17.5 mL). The mixture was stirred at rt for 1 h, then concentrated under reduced pressure to give 3-(2-(2-((S)-1-methoxyethyl)pyridin-3-yl)-5-(pyrrolidin-3-yl)-1-(2,2,2-trifluoroethyl)-1H-indol-3-yl)-2,2-dimethylpropyl acetate (7.6 g) as an oil. LCMS (ESI): m/z [M+H]⁺ calc'd for C₂₉H₃₆F₃N₃O₃ 531.3; found 532.5.
Step 4. To a mixture of 3-(2-(2-((S)-1-methoxyethyl)pyridin-3-yl)-5-(pyrrolidin-3-yl)-1-(2,2,2-trifluoroethyl)-1H-indol-3-yl)-2,2-dimethylpropyl acetate (7.7 g, 14.5 mmol) in ACN (80 mL) was added tert-butyl(S)-(2-oxooxetan-3-yl)carbamate (4.07 g, 21.7 mmol) and Cs₂CO₃ (11.80 g, 36.2 mmol). The mixture was heated to 40° C. and stirred for 2 h, then acidified to PH ˜7 with conc. HCl and the mixture was extracted with EtOAc (500 mL). The combined organic layers were washed with brine (3×100 mL), dried over anhydrous Na₂SO₄, and filtered. The filtrate was concentrated under reduced pressure and the residue was purified by preparative-HPLC to give (2S)-3-(3-(3-(3-acetoxy-2,2-dimethylpropyl)-2-(2-((S)-1-methoxyethyl)pyridin-3-yl)-1-(2,2,2-trifluoroethyl)-1H-indol-5-yl)pyrrolidin-1-yl)-2-((tert-butoxycarbonyl)amino)propanoic acid (2.3 g, 19% yield) as an oil. LCMS (ESI): m/z [M+H]⁺ calc'd for C₃₇H₄₉F₃N₄O₇ 718.4; found 719.5.
Step 5. To a mixture of methyl (S)-hexahydropyridazine-3-carboxylate (0.69 g, 4.8 mmol), DIPEA (16.54 g, 128 mmol) and (2S)-3-(3-(3-(3-acetoxy-2,2-dimethylpropyl)-2-(2-((S)-1-methoxyethyl)pyridin-3-yl)-1-(2,2,2-trifluoroethyl)-1H-indol-5-yl)pyrrolidin-1-yl)-2-((tert-butoxycarbonyl)amino)propanoic acid (2.3 g, 3.2 mmol) in DCM (60 mL) at 0° C. under an atmosphere of N₂ was added HATU (1.46 g, 3.84 mmol) in portions. The resulting mixture was warmed to rt and stirred for 1 h, the H₂O was added, and the mixture extracted with EtOAc (200 mL). The combined organic layers were washed with brine (3×400 mL), dried over anhydrous Na₂SO₄, and filtered. The filtrate was concentrated under reduced pressure and the residue was purified by preparative-HPLC to give methyl (3S)-1-((2S)-3-(3-(3-(3-acetoxy-2,2-dimethylpropyl)-2-(2-((S)-1-methoxyethyl)pyridin-3-yl)-1-(2,2,2-trifluoroethyl)-1H-indol-5-yl)pyrrolidin-1-yl)-2-((tert-butoxycarbonyl)amino)propanoyl)hexahydropyridazine-3-carboxylate (2 g, 70% yield) as an oil. LCMS (ESI): m/z [M+H]⁺ calc'd for C₄₃H₅₉F₃N₆O₈ 844.4; found 845.6.
Step 6. A mixture of methyl (3S)-1-((2S)-3-(3-(3-(3-acetoxy-2,2-dimethylpropyl)-2-(2-((S)-1-methoxyethyl)pyridin-3-yl)-1-(2,2,2-trifluoroethyl)-1H-indol-5-yl)pyrrolidin-1-yl)-2-((tert-butoxycarbonyl)amino)propanoyl)hexahydropyridazine-3-carboxylate (2.0 g, 2.4 mmol) and LiOH (0.28 g, 11.8 mmol) in H₂O (10 mL) and MeOH (20 mL) was stirred at rt. The mixture was acidified to PH ˜6 with aqueous HCl and the mixture extracted with DCM (4×mL). The combined organic layers were washed with brine (6×4 mL), dried over anhydrous Na₂SO₄, filtered and the filtrate was concentrated under reduced pressure to give (3S)-1-((2S)-2-((tert-butoxycarbonyl)amino)-3-(3-(3-(3-hydroxy-2,2-dimethylpropyl)-2-(2-((S)-1-methoxyethyl)pyridin-3-yl)-1-(2,2,2-trifluoroethyl)-1H-indol-5-yl)pyrrolidin-1-yl)propanoyl)hexahydropyridazine-3-carboxylic acid (1.9 g) as an oil. LCMS (ESI): m/z [M+H]⁺ calc'd for C₄₀H₅₅F₃N₆O₇ 788.4; found 789.4.
Step 7. To a mixture of (3S)-1-((2S)-2-((tert-butoxycarbonyl)amino)-3-(3-(3-(3-hydroxy-2,2-dimethylpropyl)-2-(2-((S)-1-methoxyethyl)pyridin-3-yl)-1-(2,2,2-trifluoroethyl)-1H-indol-5-yl)pyrrolidin-1-yl)propanoyl)hexahydropyridazine-3-carboxylic acid (1.87 g, 2.4 mmol) in DCM (340 mL) under an atmosphere of N₂ was added DIPEA (9.19 g, 71.1 mmol), HOBt (1.60 g, 11.9 mmol) and EDCl (9.09 g, 47.4 mmol). The mixture was stirred at rt overnight, then H₂O was added, and the mixture extracted with DCM (2×mL). The combined organic layers were washed with brine (3×3 mL) dried over anhydrous Na₂SO₄ and filtered. The filtrate was concentrated under reduced pressure and the residue was purified by silica gel column chromatography to give tert-butyl ((6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-pyrrolidinacycloundecaphane-4-yl)carbamate (410 mg, 21% yield) as a solid.
Step 8. Diastereomers were separated by use of silica gel column chromatography to give each respective isomer.
Data for Isomer 1 (R_{f}=0.4 in 1:1 petroleum ether/EtOAc): LCMS (ESI): m/z [M+H]⁺ calc'd for C₄₀H₅₃F₃N₆O₆ 770.4; found 771.4.
Data for Isomer 2 (R_{f}=0.7 in 1:1 petroleum ether/EtOAc): LCMS (ESI): m/z [M+H]⁺ calc'd for C₄₀H₅₃F₃N₆O₆ 770.4; found 771.4.

### Intermediate 18. Synthesis of tert-butyl ((6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-azetidinacycloundecaphane-4-yl)carbamate

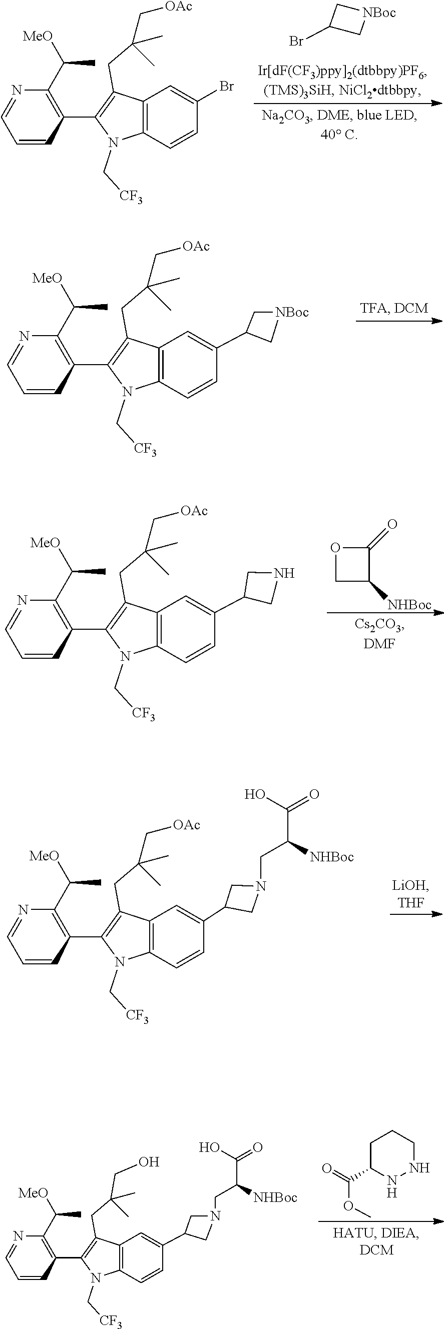

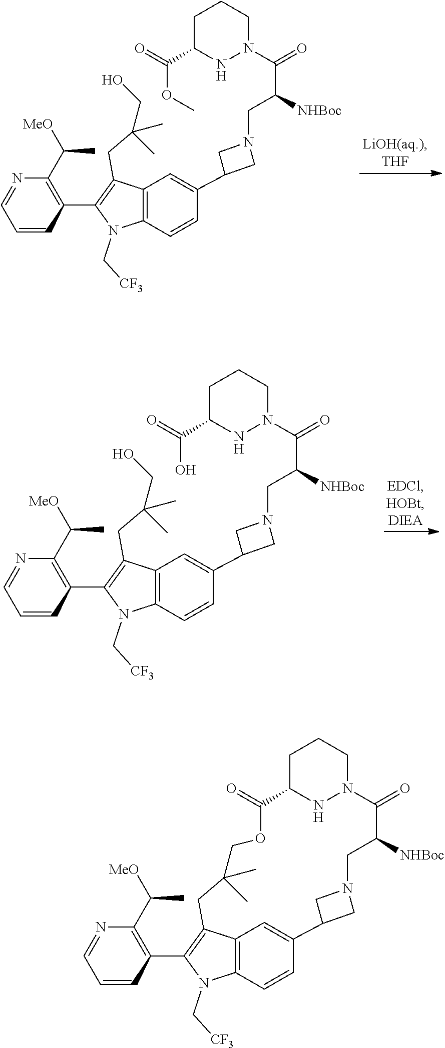

Step 1. To a 40 mL vial equipped with a stir bar was added photocatalyst Ir[dF(CF₃)ppy]₂(dtbbpy)PF₆ (62 mg, 0.055 mmol), methyl 4-bromobenzoate (1.5 g, 2.8 mmol), 4-bromotetrahydropyran (981 mg, 4.2 mmol) tris (trimethylsilyl) silane (689 mg, 2.8 mmol), and anhydrous sodium carbonate (587 mg, 5.54 mmol). The vial was sealed and placed under an atmosphere of N₂ then DME (15 mL) added. To a separate vial was added NiCl₂·glyme (6.1 mg, 0.028 mmol) and 4,4′-di-tert-butyl-2,2′-bipyridine (7.4 mg, 0.028 mmol). The catalyst vial was sealed, purged with N₂ and DME (2 mL) was added, then this mixture was sonicated 5 min, after which, the mixture was added to the photocalatyst. The mixture was degassed with N₂ for 10 min, then the mixture was sealed and stirred under irradiation from a 34 W blue LED lamp (7 cm away, with a cooling fan to keep the reaction temperature at rt. The mixture was stirred at rt for 6 h, then H₂O was added, and the mixture extracted with EtOAc (3×30 mL). The combined organic layers were washed with brine (30 mL), dried over anhydrous Na₂SO₄, and filtered. The filtrate was concentrated under reduced pressure and the crude residue was purified by silica gel column chromatography to give tert-butyl 3-[(2M)-3-[3-(acetyloxy)-2,2-dimethylpropyl]-2-{2-[(1S)-1-methoxyethyl]pyridin-3-yl}-1-(2,2,2-trifluoroethyl)indol-5-yl]azetidine-1-carboxylate (700 mg, 41% yield) as a solid. LCMS (ESI): m/z [M+H]⁺ calc'd for C₃₃H₄₂F₃N₃O₅ 617.3; found 618.4.
Step 2. To a mixture of tert-butyl 3-[(2M)-3-[3-(acetyloxy)-2,2-dimethylpropyl]-2-{2-[(1S)-1-methoxyethyl]pyridin-3-yl}-1-(2,2,2-trifluoroethyl)indol-5-yl]azetidine-1-carboxylate (800 mg, 1.3 mmol) in DCM (8 mL) at 0° C. was added TFA (2.95 g, 25.9 mmol). The mixture was warmed to rt and stirred for 2 h, then concentrated under reduced pressure and the residue was basified to PH ˜8 with saturated NaHCO₃ and extracted with EtOAc (3×30 mL). The combined organic layers were washed with brine (30 mL), dried over anhydrous Na₂SO₄, filtered and the filtrate was concentrated under reduced pressure to give 3-[(2M)-5-(azetidin-3-yl)-2-{2-[(1S)-1-methoxyethyl]pyridin-3-yl}-1-(2,2,2-trifluoroethyl)indol-3-yl]-2,2-dimethylpropyl acetate (650 mg, 97%) as a solid. LCMS (ESI): m/z [M+H]⁺ calc'd for C₂₈H₃₄F₃N₃O₃ 517.3; found 518.3.
Step 3. To a mixture of 3-[(2M)-5-(azetidin-3-yl)-2-{2-[(1S)-1-methoxyethyl]pyridin-3-yl}-1-(2,2,2-trifluoroethyl)indol-3-yl]-2,2-dimethylpropyl acetate (900 mg, 1.7 mmol) in DMF (9 mL) was added tert-butyl N-[(3S)-2-oxooxetan-3-yl]carbamate (488 mg, 2.6 mmol) and Cs₂CO₃ (567 mg, 1.7 mmol). The mixture was heated to 40° C. and stirred for 2 h, then H₂O was added, and the mixture extracted with EtOAc (3×30 mL). The combined organic layers were washed with brine (30 mL), dried over anhydrous Na₂SO₄, and filtered. After filtration, the filtrate was concentrated under reduced pressure. The filtrate was concentrated under reduced pressure and the crude residue was purified by preparative-HPLC to give (2S)-3-{3-[(2M)-3-[3-(acetyloxy)-2,2-dimethylpropyl]-2-{2-[(1S)-1-methoxyethyl]pyridin-3-yl}-1-(2,2,2-trifluoroethyl)indol-5-yl]azetidin-1-yl}-2-[(tert-butoxycarbonyl)amino]propanoic acid (400 mg, 33% yield) as a solid. LCMS (ESI): m/z [M+H]⁺ calc'd for C₃₆H₄₇F₃N₄O₇ 704.3; found 705.4.
Step 4. To a mixture of (2S)-3-{3-[(2M)-3-[3-(acetyloxy)-2,2-dimethylpropyl]-2-{2-[(1S)-1-methoxyethyl]pyridin-3-yl}-1-(2,2,2-trifluoroethyl)indol-5-yl]azetidin-1-yl}-2-[(tert-butoxycarbonyl)amino]propanoic acid (400 mg, 0.57 mmol) in THF (2.8 mL) at 0° C. was added 1M LiOH (2.84 mL, 2.84 mmol). The mixture was stirred at 0° C. for 2 h, then diluted with DCM (30 mL). The organic layer was washed with H₂O (3×30 mL) and the combined aqueous layers were acidified to PH ˜5 with 1M HCl, then extracted with EtOAc (3×40 mL). The combined organic layers were washed with brine (40 mL), dried over anhydrous Na₂SO₄, filtered and the filtrate was concentrated under reduced pressure to give (2S)-2-[(tert-butoxycarbonyl)amino]-3-{3-[(2M)-3-(3-hydroxy-2,2-dimethylpropyl)-2-{2-[(1S)-1-methoxyethyl]pyridin-3-yl}-1-(2,2,2-trifluoroethyl)indol-5-yl]azetidin-1-yl}propanoic acid (300 mg, 80%) as a solid. LCMS (ESI): m/z [M+H]⁺ calc'd for C₃₄H₄₅F₃N₄O₆ 662.3; found 663.4.
Step 5. To a mixture of (2S)-2-[(tert-butoxycarbonyl)amino]-3-{3-[(2M)-3-(3-hydroxy-2,2-dimethylpropyl)-2-{2-[(1S)-1-methoxyethyl]pyridin-3-yl}-1-(2,2,2-trifluoroethyl)indol-5-yl]azetidin-1-yl}propanoic acid (300 mg, 0.45 mmol) in DCM (3 mL) at 0° C. was added DIPEA (351 mg, 2.7 mmol), methyl (3S)-1,2-diazinane-3-carboxylate (131 mg, 0.91 mmol) and HATU (258 mg, 0.68 mmol). The mixture was stirred at 0° C. for 3 h, then H₂O was added, and the mixture extracted with DCM (3×30 mL). The combined organic layers were dried over anhydrous Na₂SO₄, filtered, the filtrate was concentrated under reduced pressure and the crude residue was purified by preparative-TLC to give methyl (3S)-1-[(2S)-2-[(tert-butoxycarbonyl)amino]-3-{3-[(2M)-3-(3-hydroxy-2,2-dimethylpropyl)-2-{2-[(1S)-1-methoxyethyl]pyridin-3-yl}-1-(2,2,2-trifluoroethyl)indol-5-yl]azetidin-1-yl}propanoyl]-1,2-diazinane-3-carboxylate (290 mg, 81%) as a solid. LCMS (ESI): m/z [M+H]⁺ calc'd for C₄₀H₅₅F₃N₆O₇ 788.4; found 789.5.
Step 6. To a mixture of methyl (3S)-1-[(2S)-2-[(tert-butoxycarbonyl)amino]-3-{3-[(2M)-3-(3-hydroxy-2,2-dimethylpropyl)-2-{2-[(1S)-1-methoxyethyl]pyridin-3-yl}-1-(2,2,2-trifluoroethyl)indol-5-yl]azetidin-1-yl}propanoyl]-1,2-diazinane-3-carboxylate (290 mg, 0.37 mmol) in THF (1.8 mL) at 0° C. was added 1M LiOH (1.84 mL, 1.84 mmol). The mixture was stirred at 0° C. for 1 h, then DCM (20 mL) was added, and the mixture washed with H₂O (3×30 mL). The combined aqueous layers were acidified to PH ˜5 with 1M HCl and the mixture was extracted with EtOAc (3×60 mL). The combined organic layers were dried over anhydrous Na₂SO₄, filtered and the filtrate was concentrated under reduced pressure to give (3S)-1-[(2S)-2-[(tert-butoxycarbonyl)amino]-3-{3-[(2M)-3-(3-hydroxy-2,2-dimethylpropyl)-2-{2-[(1S)-1-methoxyethyl]pyridin-3-yl}-1-(2,2,2-trifluoroethyl)indol-5-yl]azetidin-1-yl}propanoyl]-1,2-diazinane-3-carboxylic acid (230 mg, 81% yield) as a solid. LCMS (ESI): m/z [M+H]⁺ calc'd for C₃₉H₅₃F₃N₆O₇ 774.4; found 775.5.
Step 7. To a mixture of (3S)-1-[(2S)-2-[(tert-butoxycarbonyl)amino]-3-{3-[(2M)-3-(3-hydroxy-2,2-dimethylpropyl)-2-{2-[(1S)-1-methoxyethyl]pyridin-3-yl}-1-(2,2,2-trifluoroethyl)indol-5-yl]azetidin-1-yl}propanoyl]-1,2-diazinane-3-carboxylic acid (280 mg, 0.36 mmol) in DCM (56 mL) was added DIPEA (1.4 g, 10.8 mmol), HOBT (293 mg, 2.2 mmol) and EDCl (2.1 g, 10.8 mmol). The mixture was warmed to 30° C. and stirred overnight the H₂O was added and the mixture extracted with DCM (3×50 mL). The combined organic layers were dried over anhydrous Na₂SO₄, filtered, the filtrate was concentrated under reduced pressure and the residue was purified by preparative-TLC to give tert-butyl ((6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-azetidinacycloundecaphane-4-yl)carbamate (100 mg, 37% yield) as a solid. LCMS (ESI): m/z [M+H]⁺ calc'd for C₃₉H₅₁F₃N₆O₆ 756.4; found 757.4.

### Intermediate 19. Synthesis of tert-butyl ((6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate

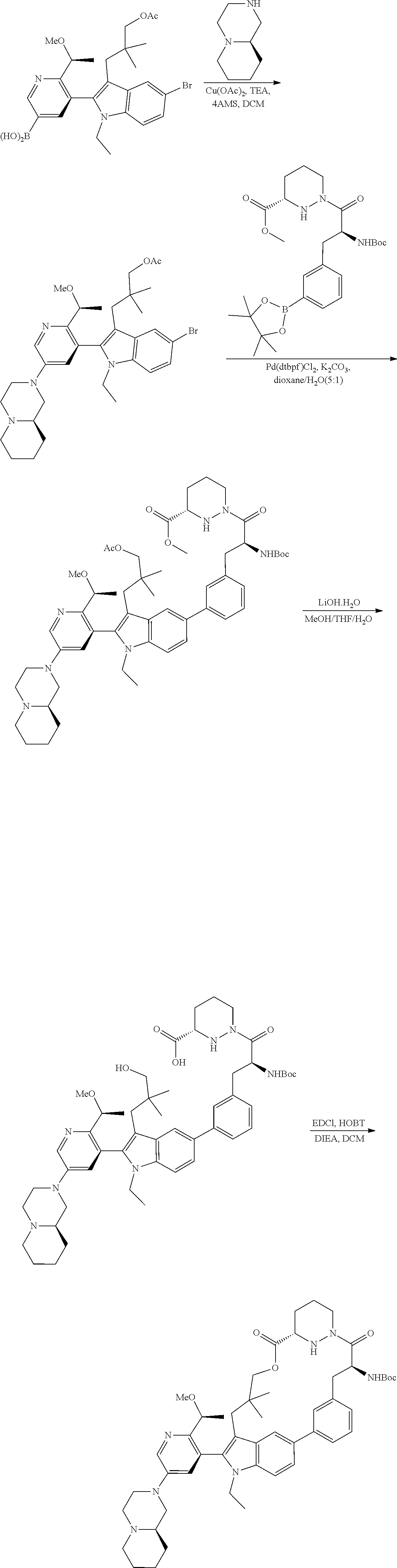

Step 1. To a mixture of (S)-(5-(3-(3-acetoxy-2,2-dimethylpropyl)-5-bromo-1-ethyl-1H-indol-2-yl)-6-(1-methoxyethyl)pyridin-3-yl)boronic acid (7.7 g, 14.5 mmol) and (R)-octahydro-2H-pyrido[1,2-a]pyrazine (3.9 g, 27.8 mmol) in DCM (230 mL) under an atmosphere of O₂ was added TEA (14.7 g, 145.3 mmol) and 4 Å molecular sieves (26 g). The mixture was stirred at rt for 30 min, then Cu (OAc) 2 (2.4 g, 13.2 mmol) was added, the mixture heated to 40° C. and stirred overnight. Ice/H₂O was added, and the mixture was extracted with EtOAc (5×200 mL). The combined organic layers were washed with brine, dried over anhydrous Na₂SO₄, and filtered. The filtrate was concentrated under reduced pressure and the residue was purified to give 3-(5-bromo-1-ethyl-2-(2-((S)-1-methoxyethyl)-5-((R)-octahydro-2H-pyrido[1,2-a]pyrazin-2-yl)pyridin-3-yl)-1H-indol-3-yl)-2,2-dimethylpropyl acetate (3.5 g, 27% yield) as a solid. LCMS (ESI): m/z [M+H]⁺ calc'd for C₃₃H₄₅BrN₄O₃ 624.3; found 625.5.
Step 2. To a mixture of 3-(5-bromo-1-ethyl-2-(2-((S)-1-methoxyethyl)-5-((R)-octahydro-2H-pyrido[1,2-a]pyrazin-2-yl)pyridin-3-yl)-1H-indol-3-yl)-2,2-dimethylpropyl acetate (1.9 g, 3.0 mmol) and methyl (S)-1-((S)-2-((tert-butoxycarbonyl)amino)-3-(3-(4,4,5,5-tetramethyl-1,3,2-dioxaborolan-2-yl)phenyl)propanoyl)hexahydropyridazine-3-carboxylate (1.89 g, 3.6 mmol) in dioxane (19 mL) and H₂O (3.8 mL) was added K₂CO₃ (1.05 g, 7.6 mmol) and Pd(dtbpf)Cl₂ (395 mg, 0.61 mmol). The mixture was heated to 70° C. and stirred for 3 h, then diluted with EtOAc (40 mL), ice/H₂O added, and the mixture was extracted with EtOAc (3×10 mL). The combined organic layers were washed with brine, dried over anhydrous Na₂SO₄, and filtered. The filtrate was concentrated under reduced pressure and the residue was purified to give methyl (S)-1-((S)-3-(3-(3-(3-acetoxy-2,2-dimethylpropyl)-1-ethyl-2-(2-((S)-1-methoxyethyl)-5-((R)-octahydro-2H-pyrido[1,2-a]pyrazin-2-yl)pyridin-3-yl)-1H-indol-5-yl)phenyl)-2-((tert-butoxycarbonyl)amino)propanoyl)hexahydropyridazine-3-carboxylate (1.1 g, 29% yield) as a solid. LCMS (ESI): m/z [M+H]⁺ calc'd for C₅₃H₇₃N₇O₈ 935.6; found 936.8.
Step 3. To a mixture of methyl (S)-1-((S)-3-(3-(3-(3-acetoxy-2,2-dimethylpropyl)-1-ethyl-2-(2-((S)-1-methoxyethyl)-5-((R)-octahydro-2H-pyrido[1,2-a]pyrazin-2-yl)pyridin-3-yl)-1H-indol-5-yl)phenyl)-2-((tert-butoxycarbonyl)amino)propanoyl)hexahydropyridazine-3-carboxylate (900 mg, 0.92 mmol) in THF (4.5 mL), MeOH (4.5 mL) and H₂O (4.5 mL) at 0° C. was added LiOH·H₂O (89 mg, 3.7 mmol). The mixture was warmed to rt and stirred for 3 h, then ice/H₂O (10 mL) added, the mixture acidified to PH ˜5 with citric acid and the mixture was extracted with EtOAc (3×20 mL). The combined organic layers were washed with brine, dried over anhydrous Na₂SO₄, filtered and the filtrate was concentrated under reduced pressure to give (S)-1-((S)-2-((tert-butoxycarbonyl)amino)-3-(3-(1-ethyl-3-(3-hydroxy-2,2-dimethylpropyl)-2-(2-((S)-1-methoxyethyl)-5-((R)-octahydro-2H-pyrido[1,2-a]pyrazin-2-yl)pyridin-3-yl)-1H-indol-5-yl)phenyl)propanoyl)hexahydropyridazine-3-carboxylic acid (900 mg) as a solid. LCMS (ESI): m/z [M+H]⁺ calc'd for C₅₀H₆₉N₇O₇ 879.5; found 880.6.
Step 4. To a mixture of (S)-1-((S)-2-((tert-butoxycarbonyl)amino)-3-(3-(1-ethyl-3-(3-hydroxy-2,2-dimethylpropyl)-2-(2-((S)-1-methoxyethyl)-5-((R)-octahydro-2H-pyrido[1,2-a]pyrazin-2-yl)pyridin-3-yl)-1H-indol-5-yl)phenyl)propanoyl)hexahydropyridazine-3-carboxylic acid (670 mg, 0.76 mmol) in DCM (67 mL) at 0° C. was added DIPEA (3.94 g, 30.4 mmol), EDCl (4.4 g, 22.8 mmol) and HOBT (514 mg, 3.8 mmol). The mixture was warmed to rt and stirred overnight, then ice/H₂O (100 mL) was added, and the mixture extracted with EtOAc (3×100 mL). The combined organic layers were washed with saturated NH₄Cl (3×100 mL), dried over anhydrous Na₂SO₄, and filtered. The filtrate was concentrated under reduced pressure and the residue was purified to give tert-butyl ((6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (450 mg, 62% yield) as a solid. LCMS (ESI): m/z [M+H]⁺ calc'd for C₅₀H₆₇N₇O₆ 861.5; found 862.7.

### Intermediate 20. Synthesis of N—((S)-3-acryloyl-4-methyl-1-oxa-3,8-diazaspiro[4.5]decane-8-carbonyl)-N-methyl-L-valine methyl ester

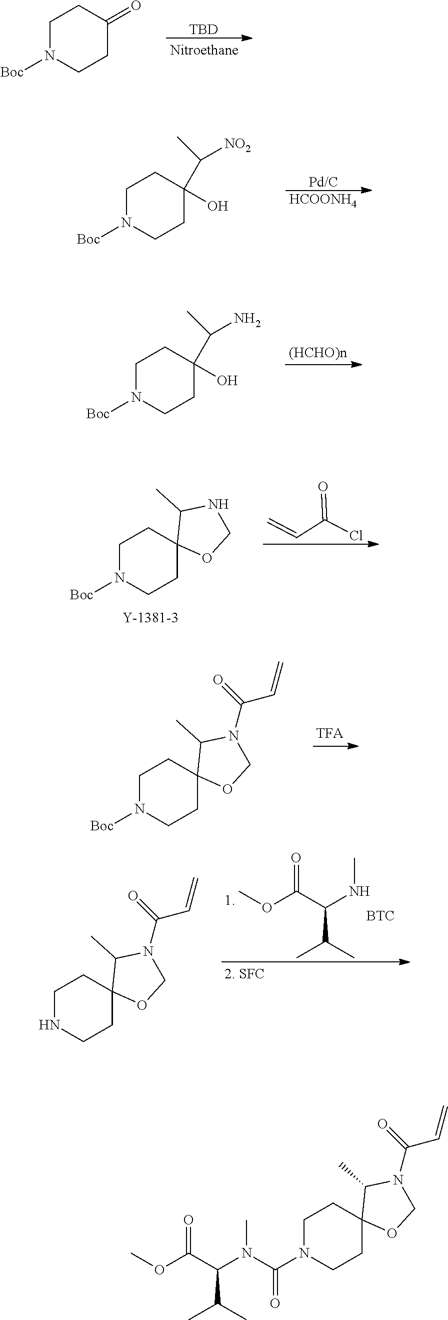

Step 1. To a solution of nitroethane (1 L) was added tert-butyl (4-oxopiperidin-1-yl) formate (200 g, 1 mol, 1 eq) and TBD (13.9 g, 0.1 mol, 0.1 eq) at 0° C. The reaction mixture was stirred at 20° C. for 16 h. The resulting mixture was concentrated under reduced pressure and the remaining residue was purified by silica gel column chromatography to afford tert-butyl 4-hydroxy-4-(1-nitroethyl) piperidine-1-carboxylate (135 g, yield 49%) as a white solid. ESI-MS m/z=299.2 [M+H]⁺, Calculated MW: 274.15.
Step 2. To a solution of tert-butyl 4-hydroxy-4-(1-nitroethyl) piperidine-1-carboxylate (135 g, 0.49 mol, 1 equiv) and HCOONH₄ (269 g, 4.3 mol, 8.7 equiv) in MeOH (1350 mL) was added Pd/C (13.6 g, 0.13 mol, 0.26 equiv) and AcOH (0.29 g, 4.9 mmol, 0.01 equiv) at room temperature. The reaction mixture was stirred for 16 h after which the mixture was adjusted to pH value of 8 with TEA (4.96 g, 0.1 equiv) and filtered. The filter cake was washed with DCM/MeOH (200 mL, 5/1). The filtrate was concentrated under reduced pressure and purified by alkaline silica gel column chromatography to afford tert-butyl 4-(1-aminoethyl)-4-hydroxypiperidine-1-carboxylate (135 g, yield 89%) as a white solid. ESI-MS m/z=189.3 [M+H-tBu]⁺, Calculated MW: 244.34.
Step 3. To a solution of [4-(1-aminoethyl)-4-hydroxypiperidin-1-yl] tert-butyl formate (40 g, 0.16 mol, 1 eq) in ACN (800 mL) was added MgSO₄ (39.1 g, 0.33 mol, 2 eq), Cs₂CO₃ (79.7 g, 0.25 mol, 1.5 eq) and (HCHO)n (19.6 g, 0.65 mol, 4 eq). The mixture was stirred at 50° C. for 2 h under N₂. The reaction mixture was filtered, and the filtrate was concentrated in vacuo to afford tert-butyl {4-methyl-1-oxa-3,8-diazaspiro[4.5]decan-8-yl} formate (40 g, yield 97%) as a colorless oil. ESI-MS m/z=257.3 [M+H]⁺, Calculated MW: 256.35.
Step 4. To a mixture of tert-butyl {4-methyl-1-oxa-3,8-diazaspiro[4.5]decan-8-yl} formate (40 g, 155.4 mmol, 1 eq) and NaHCO₃ (52.2 g, 621.6 mmol, 3 eq) in DCM (500 mL) and H₂O (500 mL) was added prop-2-enoyl chloride (15.5 g, 170.9 mmol, 1 eq) dropwise at 0° C. and stirred at 0° C. for 1 h. The resulting was filtered, and the filtrate was extracted with DCM (200 mL×2). The organic phase was washed with brine (100 mL) and concentrated under reduced pressure. The resulting residue was purified by column chromatography to afford give tert-butyl [4-methyl-3-(prop-2-enoyl)-1-oxa-3,8-diazaspiro[4.5]decan-8-yl] formate (33 g, yield 68%) as a colorless oil. ESI-MS m/z=311.1 [M+H]⁺, Calculated MW: 310.39.
Step 5. A mixture of tert-butyl [4-methyl-3-(prop-2-enoyl)-1-oxa-3,8-diazaspiro[4.5]decan-8-yl] formate (200 g, 0.64 mol, 1 equiv) in TFA/DCM (700 ml, 1/3, 2 L) was stirred for 1 hour at 0° C. The mixture was concentrated under reduced pressure at 0˜10° C. to afford crude 1-(4-methyl-1-oxa-3,8-diazaspiro[4.5]decan-3-yl)prop-2-en-1-one (350 g TFA salt, purity 36%). ESI-MS m/z=211.2 [M+H]⁺, Calculated MW: 210.28.
Step 6. To a solution of methyl (2S)-3-methyl-2-(methylamino) butanoate (63 g, 0.345 mol, 1 eq) and DIEA (360 g, 2.8 mol, 8 eq) in DCM (600 mL) was added BTC (36.5 g, 0.14 mol, 0.4 eq) in portions at 0° C., and the mixture was stirred at 0° C. for 1 h. The reaction mixture was then cooled to −40° C. and a solution of 1-{4-methyl-1-oxa-3,8-diazaspiro[4.5]decan-3-yl}prop-2-en-1-one (TFA salt, 36%, 175 g, 0.32 mol, 0.92 eq) in 300 ml DCM was added dropwise. The reaction mixture was then allowed to warm to rt and stirred for 12 h at rt. The reaction mixture was then concentrated under reduced pressure and the remaining residue was diluted with EA (0.5 L). The mixture was washed with brine (200 ml×2), dried over Na₂SO₄, and concentrated under reduced pressure to afford crude residue. The residue was purified by chromatography to afford methyl N-(3-acryloyl-4-methyl-1-oxa-3,8-diazaspiro[4.5]decane-8-carbonyl)-N-methyl-L-valinate as a racemic mixture (168 g, 64% yield). A portion of the racemic product (85 g) was separated using chiral SFC to afford N—((S)-3-acryloyl-4-methyl-1-oxa-3,8-diazaspiro[4.5]decane-8-carbonyl)-N-methyl-L-valine methyl ester ESI-MS m/z=382.2 [M+H]⁺, Calculated MW: 381.2. ¹H NMR (400 MHz, CD₃OD) δ 6.72-6.24 (m, 2H), 5.85-5.70 (m, 1H), 5.22-4.99 (m, 2H), 4.01 (d, J=6.5 Hz, 1H), 3.88 (d, J=10.4 Hz, 1H), 3.69 (s, 3H), 3.51-3.40 (m, 2H), 3.25-3.06 (m, 2H), 2.96 (s, 3H), 2.26-2.15 (m, 1H), 1.82-1.63 (m, 4H), 1.19 (dd, J=6.5, 2.3 Hz, 3H), 0.95 (dd, J=12.3, 6.6 Hz, 6H).

### Intermediate 21: Synthesis of methyl N—((R)-4-acryloyl-5-methyl-1-oxa-4,9-diazaspiro[5.5]undecane-9-carbonyl)-N-methyl-L-valinate (150 mg, 23.6%) as a yellow oil and isomer 2 methyl N—((S)-4-acryloyl-5-methyl-1-oxa-4,9-diazaspiro[5.5]undecane-9-carbonyl)-N-methyl-L-valinate

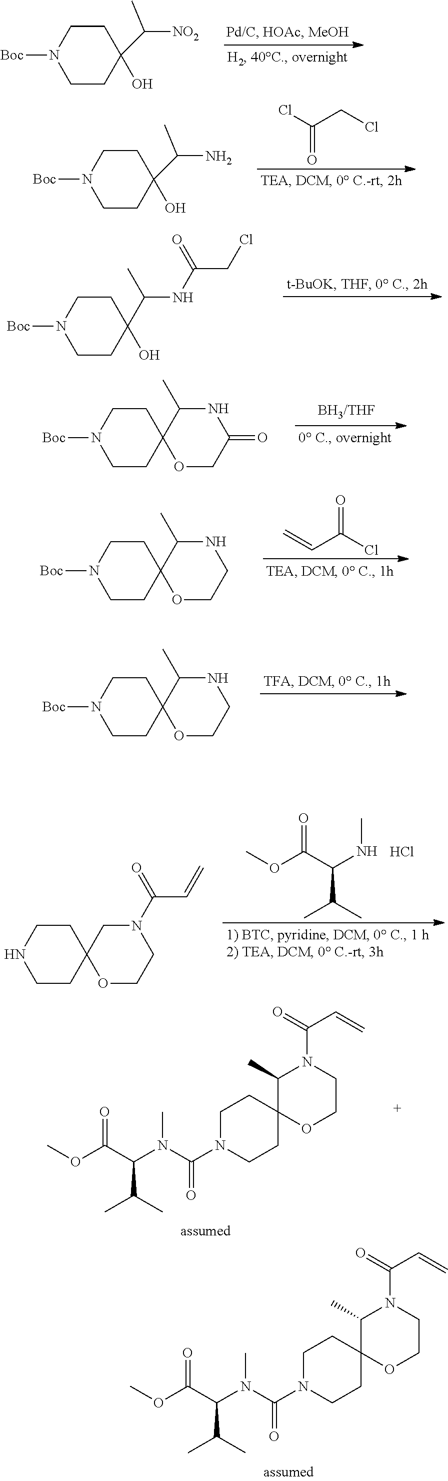

Step 1. Into a 100 mL vial were added tert-butyl 4-hydroxy-4-(1-nitroethyl) piperidine-1-carboxylate (4 g, 14.5 mmol, 1.0 equiv), Pd/C (4 g) and MeOH (40 mL) at room temperature. The resulting mixture was stirred overnight at 40° C. under hydrogen atmosphere. The resulting mixture was filtered through a pad of celite, and the filter cake was washed with MeOH/DCM=1:7 (3×50 mL). The filtrate was concentrated under reduced pressure to afford tert-butyl 4-(1-aminoethyl)-4-hydroxypiperidine-1-carboxylate (3.5 g, crude) as a light brown oil. ESI-MS m/z=489.5 [M+H]⁺; Calculated MW: 488.5.
Step 2. Into a 100 mL 3-necked round-bottom flask were added tert-butyl 4-(1-aminoethyl)-4-hydroxypiperidine-1-carboxylate (2 g, 8.1 mmol, 1 equiv), DCM (20 mL) and TEA (2.5 g, 24.7 mmol, 3 equiv) at 0° C. Chloroacetyl chloride (925 mg, 8.1 mmol, 1.0 equiv) was then add dropwise at 0° C. under argon atmosphere. The resulting mixture was stirred for 2 hours at room temperature under argon atmosphere. The reaction was quenched with water/ice and the resulting mixture was extracted with EtOAc (3×50 mL). The combined organic layers were washed with saturated sodium chloride solution (3×50 mL), dried over anhydrous Na₂SO₄, and filtered. The filtrate was concentrated under reduced pressure and the resulting residue was purified by chromatography to afford tert-butyl 4-(1-(2-chloroacetamido) ethyl)-4-hydroxypiperidine-1-carboxylate (2.19 g, 7.5%) as a light-yellow solid. ESI-MS m/z=221.2 [M+H-100]⁺; Calculated MW: 320.2.
Step 3. Into a 40 mL vial were added tert-butyl 4-(1-(2-chloroacetamido) ethyl)-4-hydroxypiperidine-1-carboxylate (1 g, 3.1 mmol, 1 equiv) and THF (10 mL) at 0° C. t-BuOK (420 mg, 3.743 mmol, 1.20 equiv) was then added in portions at 0° C. The resulting mixture was stirred for 2 hours at 0° C. under argon atmosphere. The reaction was quenched with water/ice and the resulting mixture was extracted with EtOAc (3×100 mL). The combined organic layers were washed with saturated sodium chloride solution (3×100 mL), dried over anhydrous Na₂SO₄, and filtered. The filtrate was concentrated under reduced pressure and the resulting residue was purified to afford tert-butyl 5-methyl-3-oxo-1-oxa-4,9-diazaspiro[5.5]undecane-9-carboxylate (700 mg, 64.7%) as a white solid. ESI-MS m/z=326.1 [M+H+41]+; Calculated MW: 284.2.
Step 4. Into a 40 mL vial were added tert-butyl 5-methyl-3-oxo-1-oxa-4,9-diazaspiro[5.5]undecane-9-carboxylate (450 mg, 1.5 mmol, 1 equiv) and THF (9 mL) at 0° C. To the solution was added 1M BH₃ in THF (6.8 mL) dropwise at 0° C. under argon atmosphere. The resulting mixture was stirred overnight at 0° C. under argon atmosphere. The reaction was quenched with water/ice and the mixture was basified to pH 8 with saturated NaHCO₃(aq.). The resulting mixture was extracted with EtOAc (3×50 mL), and the combined organic layers were washed with saturated sodium chloride solution (3×50 mL), dried over anhydrous Na₂SO₄, and filtered. The filtrate was concentrated under reduced pressure to afford tert-butyl 5-methyl-1-oxa-4,9-diazaspiro[5.5]undecane-9-carboxylate (500 mg, crude) as a light-yellow solid. ESI-MS m/z=271.2 [M+H]⁺; Calculated MW: 270.2.
Step 5. Into a 40 mL vial were added tert-butyl 5-methyl-1-oxa-4,9-diazaspiro[5.5]undecane-9-carboxylate (400 mg, 1.47 mmol, 1 equiv), DCM (4 mL) and TEA (450 mg, 4.4 mmol, 3 equiv) at 0° C. Acryloyl chloride (268 mg, 2.9 mmol, 2 equiv) was then added dropwise at 0° C. under argon atmosphere and stirred for 1 hour at 0° C. under argon atmosphere. The reaction was quenched with water/ice. The resulting mixture was extracted with EtOAc (3×20 mL). The combined organic layers were washed with saturated sodium chloride solution (3×10 mL), dried over anhydrous Na₂SO₄, and filtered. The filtrate was concentrated under reduced pressure and the resulting residue was purified by Prep-TLC (EA) to afford tert-butyl 5-methyl-4-(prop-2-enoyl)-1-oxa-4,9-diazaspiro[5.5]undecane-9-carboxylate (150 mg, 28.1%) as a light-yellow oil. ESI-MS m/z=325.2 [M+H]⁺; Calculated MW: 324.2.
Step 6. Into a 40 mL vial were added tert-butyl 5-methyl-4-(prop-2-enoyl)-1-oxa-4,9-diazaspiro[5.5]undecane-9-carboxylate (350 mg, 1.0 mmol, 1 equiv) and DCM (7 mL) at 0° C. TFA (3.5 mL) was then added dropwise at 0° C. under argon atmosphere. The resulting mixture was stirred for 1 hour at 0° C. under argon atmosphere after which the mixture was concentrated under reduced pressure to afford 1-(5-methyl-1-oxa-4,9-diazaspiro[5.5]undecan-4-yl)prop-2-en-1-one (400 mg, crude) as a brown oil. ESI-MS m/z=225.4 [M+H]⁺; Calculated MW: 224.2.
Step 7. Into a 40 mL vial were added Triphosgene (156 mg, 0.526 mmol, 0.33 equiv) and DCM (4 mL) at 0° C. Pyridine (380 mg, 4.8 mmol, 2.99 equiv) and methyl-L-valinate hydrochloride (300 mg, 1.65 mmol, 1.0 equiv) in DCM (4 mL) was added dropwise over 3 minutes at 0° C. The resulting mixture was stirred for additional 1 hour at 0° C. To the reaction mixture were added TEA (980 mg, 9.6 mmol, 6 equiv) and 1-(5-methyl-1-oxa-4,9-diazaspiro[5.5]undecan-4-yl)prop-2-en-1-one (360 mg, 1.605 mmol, 1 equiv) in DCM (4 mL) dropwise at 0° C. under argon atmosphere. The resulting mixture was stirred for 3 hours at room temperature under argon atmosphere. The reaction was quenched with water/ice and extracted with EtOAc (3×50 mL). The combined organic layers were washed with saturated sodium chloride solution (3×50 mL), dried over anhydrous Na₂SO₄, and filtered. The filtrate was concentrated under reduced pressure to afford the crude mixture of racemic product. The residue was purified by Chiral-HPLC to afford isomer 1 methyl N—((R)-4-acryloyl-5-methyl-1-oxa-4,9-diazaspiro[5.5]undecane-9-carbonyl)-N-methyl-L-valinate (150 mg, 23.6%) as a yellow oil and isomer 2 methyl N—((S)-4-acryloyl-5-methyl-1-oxa-4,9-diazaspiro[5.5]undecane-9-carbonyl)-N-methyl-L-valinate (110 mg, 17.3%) as a yellow oil. ESI-MS m/z=396.3 [M+H]⁺; Calculated MW: 395.2.

### Intermediate 22. Synthesis of (2S)-3-methyl-2-[methyl (4-(prop-2-enoyl)-1-oxa-4,9-diazaspiro[5.5]undecane-9-carbonyl)amino]butanoic acid

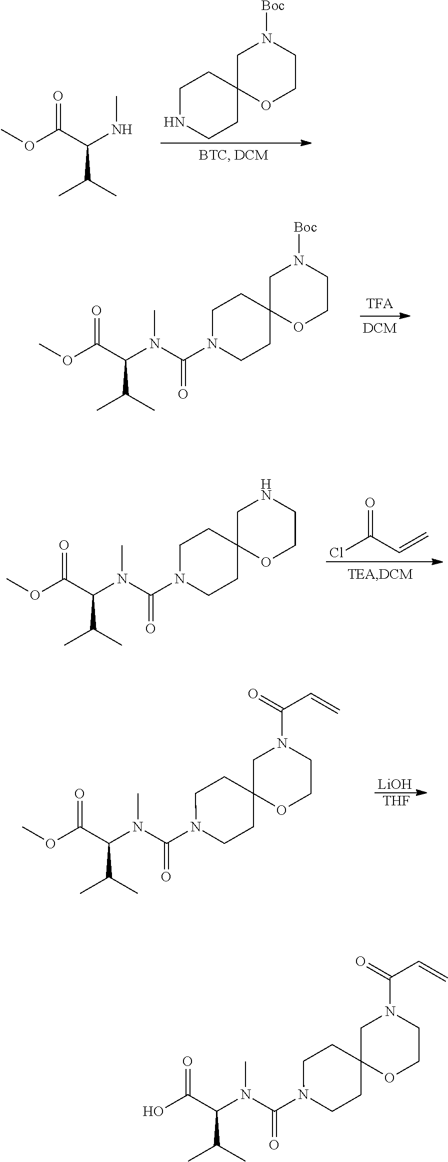

Step 1. To a mixture of ditrichloromethyl carbonate (135 mg, 0.45 mmol) and DCM (1 mL) at 0° C. was added a mixture of methyl (2S)-3-methyl-2-(methylamino) butanoate (200 mg, 1.4 mmol) and pyridine (327 mg, 4.1 mmol) in DCM (1 mL) dropwise. The mixture was stirred at 0° C. for 1 h, then tert-butyl 1-oxa-4,9-diazaspiro[5.5]undecane-4-carboxylate (353 mg, 1.4 mmol), TEA (418 mg, 4.1 mmol) in DCM (2 mL) were added dropwise at 0° C. The mixture was stirred at 0° C. for 1 h, then concentrated under reduced pressure. Brine (20 mL) was added to the residue and the mixture was extracted with DCM (3×20 mL). The combined organic layers were dried over anhydrous Na₂SO₄, filtered, the filtrate was concentrated under reduced pressure and the residue was purified by preparative-HPLC to give tert-butyl 9-{[(2S)-1-methoxy-3-methyl-1-oxobutan-2-yl](methyl)carbamoyl}-1-oxa-4,9-diazaspiro[5.5]undecane-4-carboxylate (335 mg, 57% yield) as a solid. LCMS (ESI): m/z [M+H]⁺ calc'd for C₂₁H₃₇N₃O₆ 427.3; found 428.2.
Step 2. To a mixture of tert-butyl 9-{[(2S)-1-methoxy-3-methyl-1-oxobutan-2-yl](methyl)carbamoyl}-1-oxa-4,9-diazaspiro[5.5]undecane-4-carboxylate (330 mg, 0.77 mmol) in DCM (2.4 mL) at 0° C. was added TFA (0.8 mL). The mixture was stirred at 0° C. for h, then basified to PH ˜7 with saturated NaHCO₃ and the mixture extracted with DCM (3×10 mL). The combined organic layers were washed with brine (3×10 mL), dried over anhydrous Na₂SO₄, filtered and the filtrate was concentrated under reduced pressure to give methyl (2S)-3-methyl-2-[methyl (1-oxa-4,9-diazaspiro[5.5]undecane-9-carbonyl)amino]butanoate (280 mg, crude) as a light-yellow solid. LCMS (ESI): m/z [M+H]⁺ calc'd for C₁₆H₂₉N₃O₄ 327.2; found 328.1.
Step 3. To a mixture of methyl (2S)-3-methyl-2-[methyl (1-oxa-4,9-diazaspiro[5.5]undecane-9-carbonyl)amino]butanoate (270 mg, 0.83 mmol) and TEA (1.67 g, 16.5 mmol) in DCM (3 mL) at 0° C. was added acryloyl chloride (75 mg, 0.83 mmol) dropwise. The mixture was stirred at 0° C. for 1 h, then concentrated under reduced pressure and the residue was purified by preparative-HPLC to give methyl (2S)-3-methyl-2-[methyl (4-(prop-2-enoyl)-1-oxa-4,9-diazaspiro[5.5]undecane-9-carbonyl)amino]butanoate (230 mg, 73% yield) as a solid. LCMS (ESI): m/z [M+H]⁺ calc'd for C₁₉H₃₁N₃O₅ 381.2; found 382.2.
Step 4. To a mixture of methyl (2S)-3-methyl-2-[methyl (4-(prop-2-enoyl)-1-oxa-4,9-diazaspiro[5.5]undecane-9-carbonyl)amino]butanoate (220 mg, 0.58 mmol) in THF (1.8 mL) and H₂O (0.6 mL) at 0° C. was added LiOH (21 mg, 0.87 mmol). The mixture was stirred at 0° C. for 1 day, then acidified to PH ˜4 with aqueous HCl and the mixture was extracted with DCM (3×20 mL). The combined organic layers were dried over anhydrous Na₂SO₄, filtered and the filtrate was concentrated under reduced pressure to give (2S)-3-methyl-2-[methyl (4-(prop-2-enoyl)-1-oxa-4,9-diazaspiro[5.5]undecane-9-carbonyl)amino]butanoic acid (137 mg, 65% yield) as a solid. LCMS (ESI): m/z [M+H]⁺ calc'd for C₁₈H₂₉N₃O₅ 367.2; found 368.2.

### Intermediate 23. Synthesis of methyl N—((S)-3-acryloyl-2-methyl-1-oxa-3,8-diazaspiro[4.5]decane-8-carbonyl)-N-methyl-L-valinate and methyl N—((R)-3-acryloyl-2-methyl-1-oxa-3,8-diazaspiro[4.5]decane-8-carbonyl)-N-methyl-L-valinate

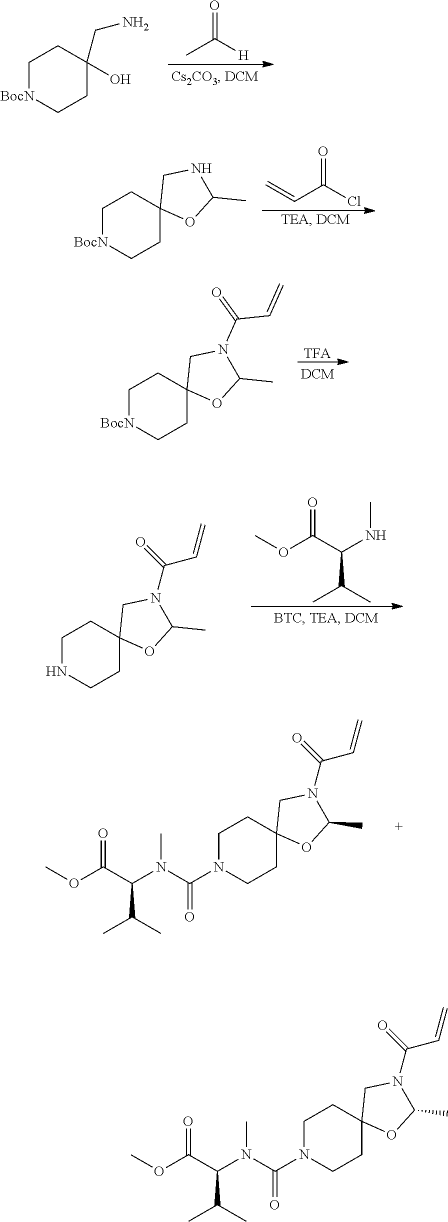

Step 1. To a mixture of tert-butyl 4-(aminomethyl)-4-hydroxypiperidine-1-carboxylate (5.0 g, 21.7 mmol) in DCM (50 mL) was added MgSO₄ (10 g), Cs₂CO₃ (7.07 g, 21.7 mmol) and acetaldehyde (0.96 g, 21.7 mmol). The mixture was stirred at rt for 2 h, then filtered and the filter cake was washed with EtOAc (5×100 mL). The filtrate was concentrated under reduced pressure to give tert-butyl 2-methyl-1-oxa-3,8-diazaspiro[4.5]decane-8-carboxylate (6 g) as an oil, which was used directly in the next step. LCMS (ESI): m/z [M+H]⁺ calc'd for C₁₃H₂₄N₂O₃ 256.2; found 257.4.
Step 2. To a mixture of tert-butyl 2-methyl-1-oxa-3,8-diazaspiro[4.5]decane-8-carboxylate (5.9 g, 23.0 mmol) in DCM (50 mL) at 0° C. was added TEA (6.99 g, 69.1 mmol) and acryloyl chloride (2.08 g, 23.0 mmol). The mixture was stirred at 0° C. for 30 min, then ice/H₂O was added, and the mixture extracted with EtOAc (4×30 mL). The combined organic layers were dried over anhydrous Na₂SO₄, filtered, the filtrate was concentrated under reduced pressure and the residue was purified by silica gel column chromatography to give tert-butyl 3-acryloyl-2-methyl-1-oxa-3,8-diazaspiro[4.5]decane-8-carboxylate (2.7 g, 38%) as an oil.
Step 3. To a mixture of tert-butyl 3-acryloyl-2-methyl-1-oxa-3,8-diazaspiro[4.5]decane-8-carboxylate (2.65 g, 8.5 mmol) in DCM (26 mL) at 0° C. was added TFA (13 mL). The mixture was stirred at 0° C. for 1 h, then concentrated under reduced pressure to give 1-(2-methyl-1-oxa-3,8-diazaspiro[4.5]decan-3-yl)prop-2-en-1-one (4.8 g) as an oil. LCMS (ESI): m/z [M+H]⁺ calc'd for C₁₁H₁₈N₂O₂ 210.1; found 211.2.
Step 4. To a mixture of BTC (0.40 g, 1.4 mmol) in DCM (10 mL) at 0° C. was added methyl methyl-L-valinate HCl (0.73 g, 4.1 mmol) and pyridine (1.28 g, 16.2 mmol) in DCM (7 mL). The mixture was stirred at 0° C. for 1 h, then TEA (4.10 g, 40.5 mmol) and 1-(2-methyl-1-oxa-3,8-diazaspiro[4.5]decan-3-yl)prop-2-en-1-one (1.70 g, 8.1 mmol) in DCM were added. The mixture was stirred at 0° C. for 2 h, then ice/H₂O was added, and the mixture extracted with EtOAc (3×20 mL). The combined organic layers were dried over anhydrous Na₂SO₄, filtered, and the filtrate was concentrated under reduced pressure. The residue was purified by preparative-TLC and preparative-HPLC to give methyl N—((S)-3-acryloyl-2-methyl-1-oxa-3,8-diazaspiro[4.5]decane-8-carbonyl)-N-methyl-L-valinate (750 mg) and methyl N—((R)-3-acryloyl-2-methyl-1-oxa-3,8-diazaspiro[4.5]decane-8-carbonyl)-N-methyl-L-valinate (730 mg) as an oil. LCMS (ESI): m/z [M+H]⁺ calc'd for C₁₉H₃₁N₃O₅ 381.2; found 382.2.

### Intermediate 24. Synthesis of (2S)-3-methyl-2-{methyl[1-methyl-3-(prop-2-enoyl)-1,3,8-triazaspiro[4.5]decan-8-yl]carbonylamino}butanoic acid, lithium salt

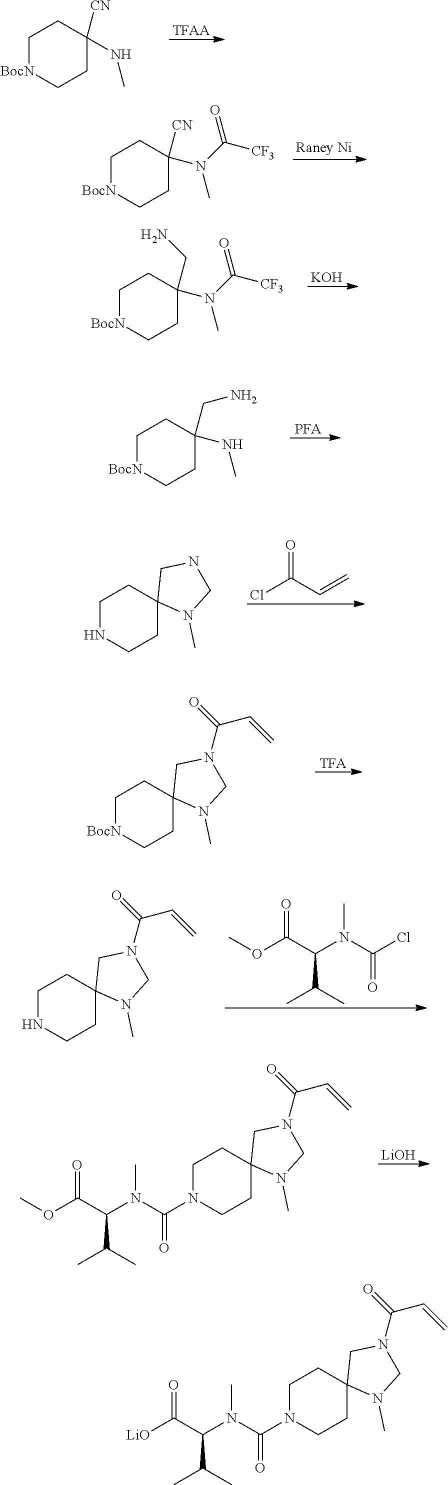

Step 1. To a mixture of tert-butyl [4-cyano-4-(methylamino) piperidin-1-yl] formate (14.4 g, 63 mmol) and pyridine (8 g, 125.6 mmol) in THF (200 mL) at 0° C. was added TFAA (15.8 g, 75.2 mmol). The mixture was warmed to rt and stirred for 1 h, then concentrated under reduced pressure. The residue was dissolved in EtOAc (100 mL), washed with 1N HCl (100 mL), then dried over Na₂SO₄ and filtered. The filtrate was concentrated under reduced pressure and the crude residue was purified by silica gel column chromatography to give tert-butyl 4-cyano-4-(2,2,2-trifluoro-N-methylacetamido) piperidine-1-carboxylate (15.9 g, 71% yield) as a solid. LCMS (ESI): m/z [M+Na]⁺ calc'd for C₁₄H₂₀F₃N₃NaO₃ 358.1; found 358.2.
Step 2. A mixture of tert-butyl 4-cyano-4-(2,2,2-trifluoro-N-methylacetamido) piperidine-1-carboxylate (9.6 g, 28 mmol) in EtOH (100 mL) and Raney Ni (2 g) was stirred under an atmosphere of H₂ (15 psi) for 16 h. The mixture was filtered, the filtrate was concentrated under reduced pressure and the crude residue was purified by silica gel column chromatography to give tert-butyl 4-(aminomethyl)-4-(2,2,2-trifluoro-1-methylacetamido) piperidine-1-carboxylate (3.9 g, 40% yield) as a solid. LCMS (ESI): m/z [M+H]⁺ calc'd for C₁₄H₂₄F₃N₃O₃ 339.2; found 340.2.
Step 3. To a mixture of tert-butyl 4-(aminomethyl)-4-(2,2,2-trifluoro-methylacetamido) piperidine-1-carboxylate (3.9 g, 12 mmol) in MeOH (40 mL) and H₂O (8 mL) was added KOH (3.45 g, 60 mmol). The mixture heated to 80° C. and stirred for 1 h, then concentrated under reduced pressure to remove MeOH. The aqueous was extracted with DCM (30 mL×3) and the combined organic layers were dried over Na₂SO₄ and filtered. The filtrate was concentrated under reduced pressure to give tert-butyl 4-(aminomethyl)-4-(methylamino) piperidine-1-carboxylate (2.9 g, 92% yield) as a solid. LCMS (ESI): m/z [M+H]⁺ calc'd for C₁₂H₂₅N₃O₂ 243.2; found 244.2.
Step 4. To a mixture of [4-(aminomethyl)-4-(methylamino) piperidin-1-yl] tert-butyl formate (1.4 g, 5.7 mmol) in Et₂O (15 mL) was added paraformaldehyde (0.77 g, 25.6 mmol). The mixture was stirred at rt for 1 h, then filtered and the filter cake washed with DCM. The filtrate was concentrated under reduced pressure to give tert-butyl {1-methyl-1,3,8-triazaspiro[4.5]decan-8-yl} formate (1.2 g, 77% yield) as an oil. LCMS (ESI): m/z [M+H]⁺ calc'd for C₁₃H₂₅N₃O₂ 255.2; found 256.3.
Step 5. To a mixture of tert-butyl {1-methyl-1,3,8-triazaspiro[4.5]decan-8-yl} formate (1.4 g, 5.5 mmol), NaHCO₃ (1.16 g, 13.7 mmol) in H₂O (15 mL) and DCM (15 mL) at 0° C. was added prop-2-enoyl chloride (0.55 g, 6 mmol). The mixture was stirred at 0° C. for 1H, then H₂O (30 mL) added, and the mixture was extracted with DCM (50 mL×3). The obtained organic layers were washed with brine, dried over anhydrous Na₂SO₄, and filtered. The filtrate was concentrated under reduced pressure and the crude residue was purified by silica gel column chromatography to give tert-butyl [1-methyl-3-(prop-2-enoyl)-1,3,8-triazaspiro[4.5]decan-8-yl] formate (0.8 g, 43% yield) as an oil. LCMS (ESI): m/z [M+H]⁺ calc'd for C₁₆H₂₇N₃O₃ 309.2; found 310.3.
Step 6. To a mixture of tert-butyl [1-methyl-3-(prop-2-enoyl)-1,3,8-triazaspiro[4.5]decan-8-yl] formate (800 mg, 2.6 mmol) in DCM (6 mL) was added TFA (2 mL). The mixture was stirred at rt for 1 h then concentrated under reduced pressure to give 1-{1-methyl-1,3,8-triazaspiro[4.5]decan-3-yl}prop-2-en-1-one (540 mg), which was used directly in the next step. LCMS (ESI): m/z [M+H]⁺ calc'd for C₁₁H₁₉N₃O 209.2; found 210.3.
Step 7. To a mixture of 1-{1-methyl-1,3,8-triazaspiro[4.5]decan-3-yl}prop-2-en-1-one (540 mg, 2.6 mmol) and methyl (2S)-2-[(chlorocarbonyl)(methyl)amino]-3-methylbutanoate (589 mg, 2.83 mmol) in DCM (10 mL) at 0° C. was added TEA (781 mg, 7.74 mmol). The mixture was stirred at 0° C. for 1 h, then H₂O (30 mL) added, and the mixture was extracted with DCM (50 mL×3). The obtained organic layers were washed with brine, dried over anhydrous Na₂SO₄, and filtered. The filtrate was concentrated under reduced pressure and the crude residue was purified by silica gel column chromatography to give methyl (2S)-3-methyl-2-{methyl[1- an oil. LCMS (ESI): m/z [M+H]⁺ calc'd for C₁₉H₃₂N₄O₄ 380.2; found 381.3.
Step 8. To a mixture of methyl (2S)-3-methyl-2-{methyl[1-methyl-3-(prop-2-enoyl)-1,3,8-triazaspiro[4.5]decan-8-yl]carbonylamino]butanoate (600 mg, 1.6 mmol) in THF (3 mL) was added LiOH (75.5 mg, 3.15 mmol) in H₂O (2 mL). The mixture was stirred at rt for 1 h, then lyophilized to afford (2S)-3-methyl-2-{methyl[1-methyl-3-(prop-2-enoyl)-1,3,8-triazaspiro[4.5]decan-8-yl]carbonylamino}butanoic acid, lithium salt (500 mg, 78% yield) as a solid. LCMS (ESI): m/z [M+H]⁺ calc'd for C₁₈H₃₀N₄O₄ 366.2; found 367.2.

### Intermediate 25. Synthesis of (2S)-3-methyl-2-{methyl[3-(prop-2-enoyl)-1-oxa-3,8-diazaspiro[4.5]decan-8-yl]carbonylamino}butanoic acid

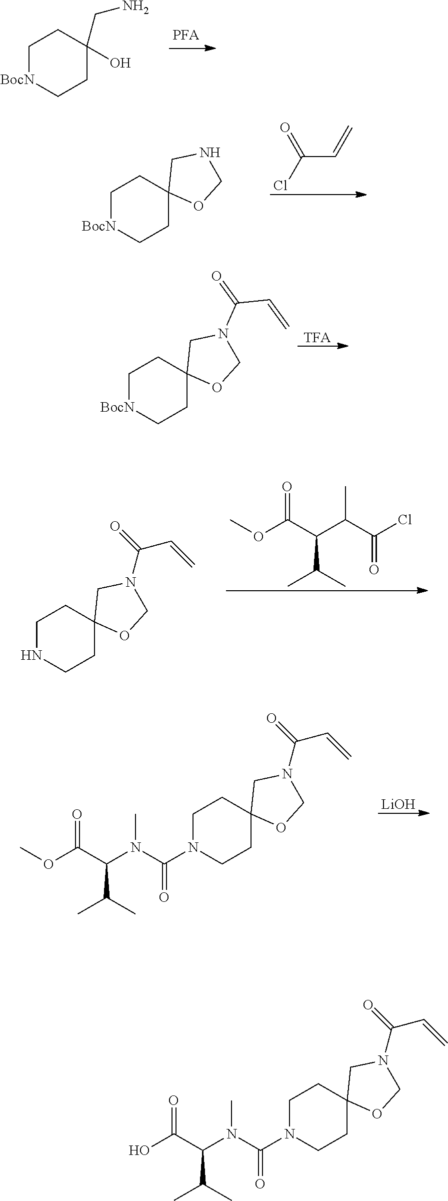

Step 1. To a mixture of tert-butyl 4-(aminomethyl)-4-hydroxypiperidine-1-carboxylate (26 g, 112.9 mmol) in MeOH (52 mL) and 3M NaOH (260 mL) was added HCHO (37 wt. % in H₂O; 52 mL). The mixture was stirred for stirred at rt for 16 h, then extracted with DCM (100 mL×3). The combined organic layers were dried over Na₂SO₄, filtered, and concentrated under reduced pressure to give tert-butyl 1-oxa-3,8-diazaspiro[4.5]decane-8-carboxylate (28.8 g) as an oil. The crude product was used directly in the next step. LCMS (ESI): m/z [M+H]⁺ calc'd for C₁₂H₂₂N₂O₃ 242.2; found 243.2.
Step 2. To a mixture of tert-butyl 1-oxa-3,8-diazaspiro[4.5]decane-8-carboxylate (14.4 g, 59.4 mmol) and NaHCO₃ (14.97 g, 178.2 mmol) in DCM (75 mL) and H₂O (75 mL) at 0° C. was added prop-2-enoyl chloride (8.06 g, 89.1 mmol). The mixture was stirred at 0° C. for 1 h, then extracted with DCM (50 mL×3). The combined organic layers were concentrated under reduced pressure and the crude residue was purified by silica gel column chromatography to give tert-butyl 3-(prop-2-enoyl)-1-oxa-3,8-diazaspiro[4.5]decane-8-carboxylate (10 g, 54% yield) as an oil. LCMS (ESI): m/z [M+H]⁺ calc'd for C₁₅H₂₄N₂O₄ 296.2; found 297.2.
Step 3. To a mixture of tert-butyl 3-(prop-2-enoyl)-1-oxa-3,8-diazaspiro[4.5]decane-8-carboxylate (1.0 g, 3.4 mmol) in DCM (6 mL) was added TFA (2 mL). The mixture was stirred at rt for 1 h, then concentrated under reduced pressure to give 1-{1-oxa-3,8-diazaspiro[4.5]decan-3-yl}prop-2-en-1-one (0.67 g) as an oil. The product was used to next step directly. LCMS (ESI): m/z [M+H]⁺ calc'd for C₁₀H₁₆N₂O₂ 196.1; found 197.1.
Step 4. To a mixture of methyl (2S)-2-[(chlorocarbonyl)amino]-3-methylbutanoate (0.66 g, 3.4 mmol) and TEA (1.72 g, 17 mmol) in DCM (10 mL) at 0° C. was added 1-{1-oxa-3,8-diazaspiro[4.5]decan-3-yl}prop-2-en-1-one (0.67 g, 3.4 mmol). The mixture was stirred at 0° C. for 1 h, then H₂O (30 mL) added, and the mixture was extracted with DCM (30 mL). The combined organic layers were concentrated under reduced pressure and the crude residue was purified by silica gel column chromatography to give methyl (2S)-3-methyl-2-{methyl[3-(prop-2-enoyl)-1-oxa-3,8-diazaspiro[4.5]decan-8-yl]carbonylamino}butanoate (600 mg, 47% yield) as an oil. LCMS (ESI): m/z [M+H]⁺ calc'd for C₁₈H₂₉N₃O₅ 367.2; found 368.3.
Step 5. To a mixture of methyl (2S)-3-methyl-2-{methyl[3-(prop-2-enoyl)-1-oxa-3,8-diazaspiro[4.5]decan-8-yl]carbonylamino}butanoate (600 mg, 1.63 mmol) in THF (5 mL) was added a solution of lithium hydroxide (78 mg, 3.3 mmol) in H₂O (5 mL). The mixture was stirred at rt for 4 h, then adjusted to PH ˜4 with 1N HCl, and extracted with DCM (20 mL×3). The combined organic layers were concentrated under reduced pressure to give (2S)-3-methyl-2-{methyl[3-(prop-2-enoyl)-1-oxa-3,8-diazaspiro[4.5]decan-8-yl]carbonylamino}butanoic acid (500 mg) as an oil. LCMS (ESI): m/z [M+H]⁺ calc'd for C₁₇H₂₇N₃O₅ 353.2; found 354.2.

### Intermediate 26. Synthesis of (2S)-3-methyl-2-{methyl[4-(prop-2-enoyl)-1-propyl-1,4,9-triazaspiro[5.5]undecan-9-yl]carbonylamino}butanoic acid, lithium salt

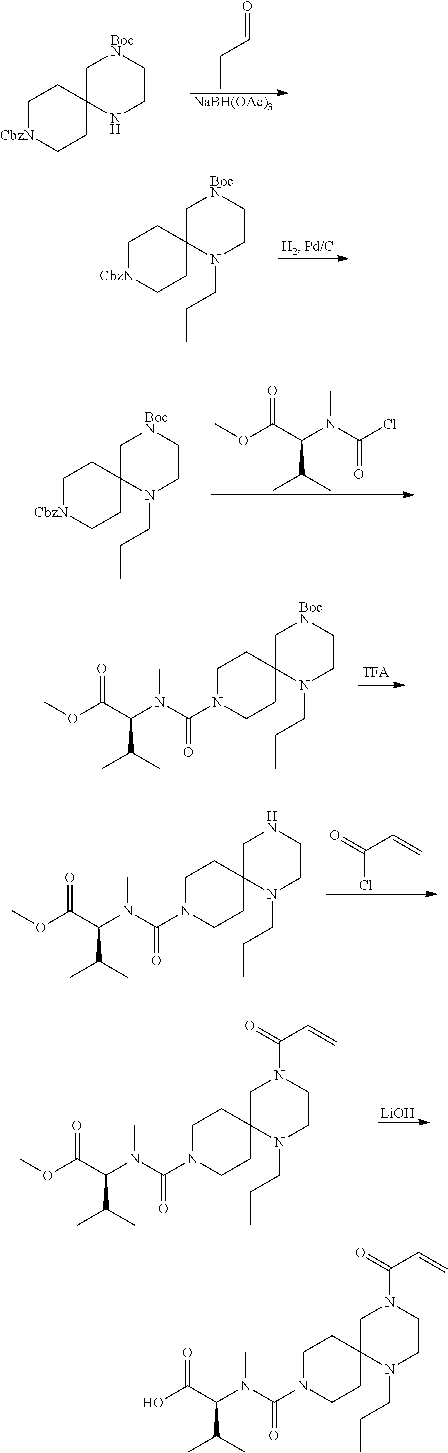

Step 1. To a mixture of tert-butyl 9-{3-[(formyloxy)methyl]phenyl}-1,4,9-triazaspiro[5.5]undecane-4-carboxylate (1.0 g, 2.6 mmol) and propanal (0.3 g, 5.2 mmol) in DCM (10 mL) was stirred at rt for 20 min. NaBH(OAc)₃ (1.1 g, 5.2 mmol) was added and the mixture was stirred at rt for 1 h, then H₂O (20 mL) added and the mixture was extracted with DCM (20 mL×3). The combined organic layers were washed with brine (20 mL), dried over anhydrous Na₂SO₄, and filtered. The filtrate was concentrated under reduced pressure and the crude residue was purified by silica gel column chromatography to give tert-butyl 9-{3-[(formyloxy)methyl]phenyl}-1-propyl-1,4,9-triazaspiro[5.5]undecane-4-carboxylate (0.7 g, 62% yield) as an oil. LCMS (ESI): m/z [M+H]⁺ calc'd for C₂₄H₃₇N₃O₄ 431.3; found 432.3.
Step 2. A mixture of tert-butyl 9-{3-[(formyloxy)methyl]phenyl}-1-propyl-1,4,9-triazaspiro[5.5]undecane-4-carboxylate (600 mg, 1.39 mmol) and 10% Pd/C (148 mg, 1.39 mmol) in THF (10 mL) was stirred under an atmosphere of H₂ (15 psi) at rt for 1 h. The mixture was filtered, and the filtrate was concentrated under reduced pressure to give tert-butyl 1-propyl-1,4,9-triazaspiro[5.5]undecane-4-carboxylate (500 mg) as an oil. LCMS (ESI): m/z [M+H]⁺ calc'd for C₁₆H₃₁N₃O₂ 297.2; found 298.2.
Step 3. To a mixture of methyl (2S)-2-[(chlorocarbonyl)(methyl)amino]-3-methylbutanoate (314 mg, 1.5 mmol) in DCM (5 mL) at 0° C. was added TEA (458 mg, 4.5 mmol) and tert-butyl 1-propyl-1,4,9-triazaspiro[5.5]undecane-4-carboxylate (450 mg, 1.5 mmol). The mixture was stirred at 0° C. for 1 h, then H₂O (20 mL) added, and the mixture was extracted with DCM (20 mL×3). The combined organic layers were washed with brine (20 mL), dried over anhydrous Na₂SO₄, and filtered. The filtrate was concentrated under reduced pressure and the crude residue was purified by silica gel column chromatography to give tert-butyl 9-{[(2S)-1-methoxy-3-methyl-1-oxobutan-2-yl](methyl)carbamoyl}-1-propyl-1,4,9-triazaspiro[5.5]undecane-4-carboxylate (650 mg, 83% yield) as an oil. LCMS (ESI): m/z [M+H]⁺ calc'd for C₂₄H₄₄N₄O₅ 468.3; found 469.3.
Step 4. To a mixture of tert-butyl 9-{[(2S)-1-methoxy-3-methyl-1-oxobutan-2-yl](methyl)carbamoyl}-1-propyl-1,4,9-triazaspiro[5.5]undecane-4-carboxylate (550 mg, 1.17 mmol) in DCM (6 mL) at 0° C. was added TFA (2 mL). The mixture was stirred at 0° C. for 15 min, then concentrated under reduced pressure to give methyl (2S)-3-methyl-2-[methyl ({1-propyl-1,4,9-triazaspiro[5.5]undecan-9-yl}carbonyl)amino]butanoate (435 mg), that was used directly in the next step. LCMS (ESI): m/z [M+H]⁺ calc'd for C₁₉H₃₆N₄O₃ 368.3; found 369.3.
Step 5. To a mixture of methyl (2S)-3-methyl-2-[methyl ({1-propyl-1,4,9-triazaspiro[5.5]undecan-9-yl}carbonyl)amino]butanoate (435 mg, 1.18 mmol) in DCM (5 mL) and H₂O (5 mL) at 0° C. was added NaHCO₃ (991 mg, 11.8 mmol) and prop-2-enoyl chloride (214 mg, 2.36 mmol). The mixture was stirred at 0° C. for 1 h, then H₂O (20 mL) added, and the mixture was extracted with DCM (20 mL×3). The combined organic layers were washed with brine (20 mL), dried over anhydrous Na₂SO₄, and filtered. The filtrate was concentrated under reduced pressure and the crude residue was purified by silica gel column chromatography to give methyl (2S)-3-methyl-2-{methyl[4-(prop-2-enoyl)-1-propyl-1,4,9-triazaspiro[5.5]undecan-9-yl]carbonylamino}butanoate (460 mg, 83% yield) as an oil. LCMS (ESI): m/z [M+H]⁺ calc'd for C₂H₃₈N₄O₄ 422.3; found 423.3.
Step 6. To a mixture of methyl (2S)-3-methyl-2-{methyl[4-(prop-2-enoyl)-1-propyl-1,4,9-triazaspiro[5.5]undecan-9-yl]carbonylamino]butanoate (100 mg, 0.24 mmol) in THF (1 mL) was added a mixture of LiOH (11.3 mg, 0.47 mmol) in H₂O (1.5 mL). The mixture was stirred at rt for 4 h, then lyophilized to afford (2S)-3-methyl-2-{methyl[4-(prop-2-enoyl)-1-propyl-1,4,9-triazaspiro[5.5]undecan-9-yl]carbonylamino}butanoic acid, lithium salt (96 mg) as a solid, that was used directly in the next step. LCMS (ESI): m/z [M+H]⁺ calc'd for C₂₁H₃₆N₄O₄ 408.3; found 409.3.

### Intermediate 27. Synthesis of methyl N-((2S,4R)-3-acryloyl-2,4-dimethyl-1-oxa-3,8-diazaspiro[4.5]decane-8-carbonyl)-N-methyl-L-valinate (52 mg) and methyl N-((2R,4R)-3-acryloyl-2,4-dimethyl-1-oxa-3,8-diazaspiro[4.5]decane-8-carbonyl)-N-methyl-L-valinate

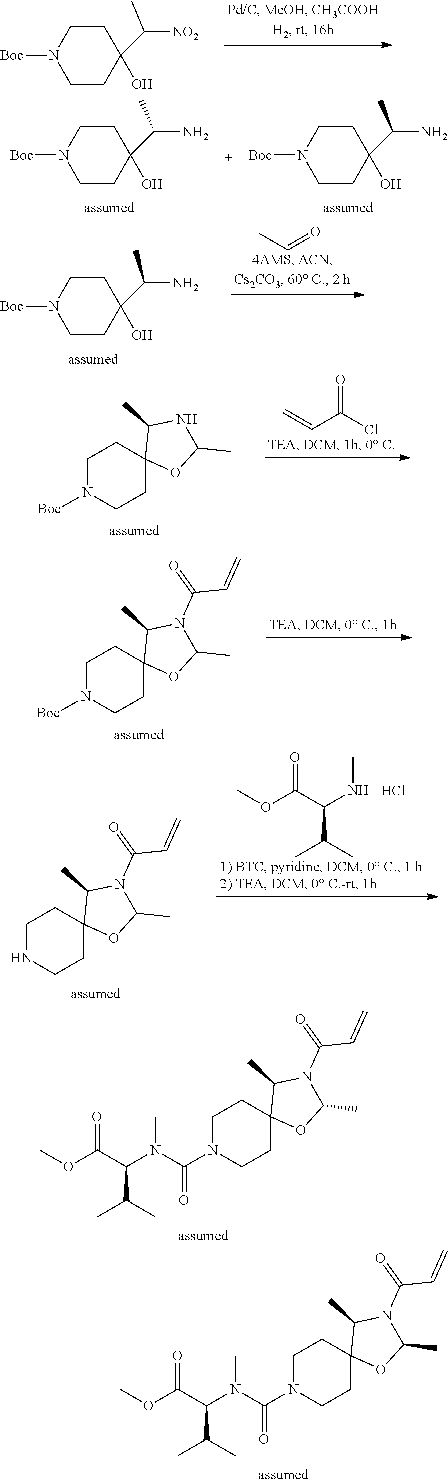

Step 1. Into a 500 mL 3-necked round-bottom flask were added tert-butyl 4-hydroxy-4-(1-nitroethyl) piperidine-1-carboxylate (20 g, 72.9 mmol, 1.0 equiv), Pd/C (20 g, 187.9 mmol, 2.6 equiv) and MeOH (100 mL, 2469.9 mmol, 33.9 equiv) at room temperature. The resulting mixture was stirred overnight at room temperature under hydrogen atmosphere. The resulting mixture was filtered through a pad of celite, and the filter cake was washed with 3×100 mL of MeOH:DCM=1:7. The resulting mixture was concentrated under reduced pressure and the resulting residue was purified by chromatography to afford tert-butyl 4-(1-aminoethyl)-4-hydroxypiperidine-1-carboxylate (10.8 g, 60.63%) as a brown solid. The racemic product (6.8 g) was purified by Chiral SFC to afford tert-butyl (S)-4-(1-aminoethyl)-4-hydroxypiperidine-1-carboxylate (3 g) and tert-butyl (R)-4-(1-aminoethyl)-4-hydroxypiperidine-1-carboxylate (2.9 g) as a yellow oil. ESI-MS m/z=245.3 [M+H]⁺; Calculated MW: 244.2.
Step 2. Into a 100 mL 3-necked round-bottom flask were added tert-butyl (R)-4-(1-aminoethyl)-4-hydroxypiperidine-1-carboxylate (2.9 g, 12.0 mmol, 1.0 equiv), 4AMS (2.9 g), Cs₂CO₃ (7.8 g, 24.0 mmol, 2 equiv), ACN (30 mL) and acetaldehyde (0.53 g, 12.0 mmol, 1 equiv) at 0° C. The resulting mixture was stirred for 4 hours at 60° C. The resulting mixture was filtered, and the filter cake was washed with ethyl acetate (3×10 mL). The filtrate was concentrated under reduced pressure to afford tert-butyl (4R)-2,4-dimethyl-1-oxa-3,8-diazaspiro[4.5]decane-8-carboxylate (2.63 g, crude) as a light-yellow solid. The crude product was used in the next step directly without further purification. ESI-MS m/z=271.4 [M+H]⁺; Calculated MW: 270.2.
Step 3. Into a 100 mL round-bottom flask were added tert-butyl (4R)-2,4-dimethyl-1-oxa-3,8-diazaspiro[4.5]decane-8-carboxylate (2.5 g, 9.3 mmol, 1.0 equiv), DCM (25 mL), acryloyl chloride (845.5 mg, 9.3 mmol, 1 equiv) and TEA (2.8 g, 28.1 mmol, 3 equiv) at 0° C. The resulting mixture was stirred for 30 mins at 0° C. The reaction was quenched with water/ice at 0° C. The resulting mixture was extracted with EtOAc (4×50 mL), and the combined organic layers were dried over anhydrous Na₂SO₄. After filtration, the filtrate was concentrated under reduced pressure and the resulting residue was purified by chromatography to afford tert-butyl (4R)-2,4-dimethyl-3-(prop-2-enoyl)-1-oxa-3,8-diazaspiro[4.5]decane-8-carboxylate (1.5 g, 49.2%) as a yellow oil. ESI-MS m/z=269.4 [M-55]⁺; Calculated MW: 324.2.
Step 4. Into a 40 mL vial were added tert-butyl (4S)-3-acryloyl-2,4-dimethyl-1-oxa-3,8-diazaspiro[4.5]decane-8-carboxylate (1.4 g, 4.4 mmol, 1.0 equiv) and DCM (14.5 mL) at room temperature. TFA (7.2 mL, 97.6 mmol, 21.8 equiv) was the added dropwise over 5 mins at 0° C. and the resulting mixture was stirred for additional 1 hour at 0° C. The mixture was concentrated under reduced pressure to afford 1-((4S)-2,4-dimethyl-1-oxa-3,8-diazaspiro[4.5]decan-3-yl)prop-2-en-1-one (3.5 g, crude) as a yellow oil. ESI-MS m/z=225.1 [M+H]⁺; Calculated MW: 224.2.
Step 5. To a stirred solution of BTC (402 mg, 1.3 mmol, 0.16 equiv) in DCM (9 mL) was added the mixture of methyl methyl-L-valinate hydrochloride (614 mg, 4.2 mmol, 0.5 equiv) and Pyridine (1340 mg, 16.9 mmol, 2 equiv) in DCM (8 ml) at 0° C. The resulting mixture was stirred for 1 hour at 0° C. after which TEA (4285 mg, 42.3 mmol, 5 equiv) and 1-[(4R)-2,4-dimethyl-1-oxa-3,8-diazaspiro[4.5]decan-3-yl]prop-2-en-1-one (1.9 g, 8.4 mmol, 1 equiv) in DCM were added at 0° C. The resulting mixture was stirred for additional 2 hours at 0° C. and then quenched with water/ice at 0° C. The resulting mixture was extracted with EtOAc (4×30 mL), and the combined organic layers were dried over anhydrous Na₂SO₄ and filtered. The filtrate was concentrated under reduced pressure and the resulting residue was purified by chromatography to afford racemic methyl N-((4S)-3-acetyl-2,4-dimethyl-1-oxa-3,8-diazaspiro[4.5]decane-8-carbonyl)-N-methyl-L-valinate (1.2 g, 32% yield) as a yellow oil. The racemic product (1.2 g) was purified by Chiral-HPLC afford methyl N-((2S,4R)-3-acryloyl-2,4-dimethyl-1-oxa-3,8-diazaspiro[4.5]decane-8-carbonyl)-N-methyl-L-valinate (52 mg) and methyl N-((2R,4R)-3-acryloyl-2,4-dimethyl-1-oxa-3,8-diazaspiro[4.5]decane-8-carbonyl)-N-methyl-L-valinate (0.9 g) as a yellow oil. ESI-MS m/z=396.0 [M+H]⁺; Calculated MW: 395.2

### Example 1. Synthesis of 1-(4-(dimethylamino)-4-methylpent-2-ynoyl)-N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(5,3)-oxadiazola-1(5,3)-indola-6(1,3)-pyridazinacycloundecaphane-4-yl)amino)-3-methyl-1-oxobutan-2-yl)-4-fluoro-N-methylpiperidine-4-carboxamide (A118)

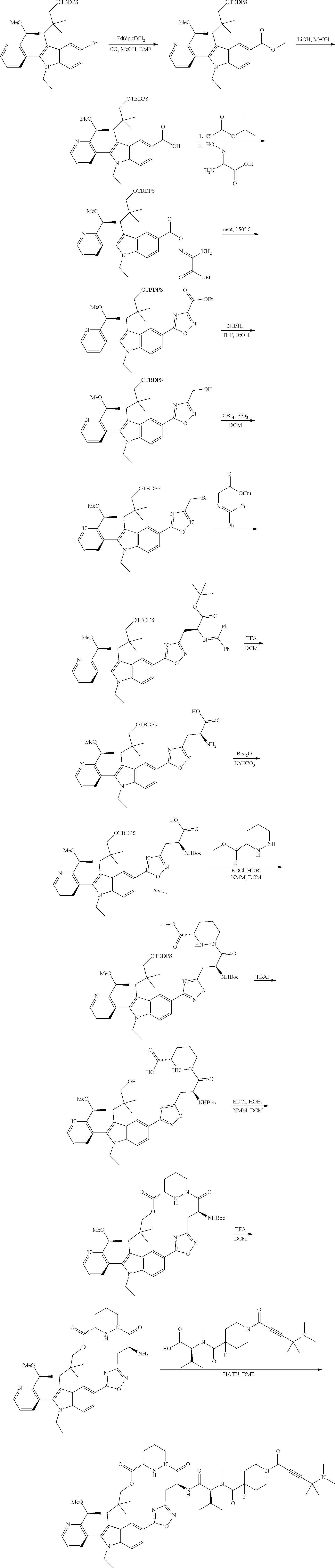

Step 1. A mixture of (2M)-5-bromo-3-[3-[(tert-butyldiphenylsilyl)oxy]-2,2-dimethylpropyl]-1-ethyl-2-[2-[(1S)-1-methoxyethyl]pyridin-3-yl]indole (10.0 g, 14.6 mmol), Pd(dppf)Cl₂·DCM (1.19 g, 1.46 mmol) and TEA (2.66 g, 26.3 mmol) in DMF (50 mL) and MeOH (1 mL) under an atmosphere of CO was heated too 100° C. and stirred overnight. H₂O (100 mL) was added, and the mixture extracted with EtOAc (3×100 mL). The combined organic layers were dried over anhydrous Na₂SO₄ and filtered. The filtrate was concentrated under reduced pressure and the residue was purified by silica gel column chromatography to give (2M)-3-[3-[(tert-butyldiphenylsilyl)oxy]-2,2-dimethylpropyl]-1-ethyl-2-[2-[(1S)-1-methoxyethyl]pyridin-3-yl]indole-5-carboxylate (8.0 g, 74% yield) as a foam. LCMS (ESI): m/z [M+H]⁺ calc'd for C₄₁H₅₀N₂O₄Si 662.4; found 663.4.
Step 2. To a mixture of (2M)-3-[3-[(tert-butyldiphenylsilyl)oxy]-2,2-dimethylpropyl]-1-ethyl-2-[2-[(1S)-1-methoxyethyl]pyridin-3-yl]indole-5-carboxylate (3.90 g, 5.9 mmol) in THF (10 mL) and MeOH (30 mL) at 0° C. was added LiOH (0.70 g, 29.2 mmol) in H₂O (30 mL) dropwise. The mixture was warmed to rt and stirred for 3 h, then acidified to PH ˜7 with aqueous HCl and the mixture extracted with EtOAc (3×20 mL). The combined organic layers were washed with brine (2×20 mL), dried over anhydrous Na₂SO₄, filtered and the filtrate was concentrated under reduced pressure to give (2M)-3-[3-[(tert-butyldiphenylsilyl)oxy]-2,2-dimethylpropyl]-1-ethyl-2-[2-[(1S)-1-methoxyethyl]pyridin-3-yl]indole-5-carboxylic acid (2.89 g) as a solid. LCMS (ESI): m/z [M+H]⁺ calc'd for C₄₀H₄₈N₂O₄Si 648.3; found 649.3.
Step 3. To a mixture of (2M)-3-[3-[(tert-butyldiphenylsilyl)oxy]-2,2-dimethylpropyl]-1-ethyl-2-[2-[(1S)-1-methoxyethyl]pyridin-3-yl]indole-5-carboxylic acid (2.00 g, 3.1 mmol) and K₂CO₃ (0.85 g, 6.2 mmol) in DCM (20 mL) at 0° C. was added isopropyl chloroformate (0.76 g, 6.2 mmol) dropwise. The mixture was stirred at rt for 45 min, then H₂O was added, and the mixture was extracted with EtOAc (3×50 mL). The combined organic layers were dried over anhydrous Na₂SO₄, filtered and the filtrate was concentrated under reduced pressure. The residue was dissolved in DCM (20 mL) and ethyl [(Z)—N-hydroxycarbamimidoyl] formate (0.81 g, 6.2 mmol) and K₂CO₃ (0.85 g, 6.2 mmol) were added. The mixture was stirred at rt for 2 h, then H₂O was added, and the mixture was extracted with EtOAc (3×30 mL). The combined organic layers were dried over anhydrous Na₂SO₄ and filtered. The filtrate was concentrated under reduced pressure and the residue was purified by preparative-HPLC to give ethyl [(Z)—N—[(Z)-(2M)-3-[3-[(tert-butyldiphenylsilyl)oxy]-2,2-dimethylpropyl]-1-ethyl-2-[2-[(1S)-1-methoxyethyl]pyridin-3-yl]indole-5-carbonyloxy] carbamimidoyl] formate (1.23 g, 45% yield) as a solid. LCMS (ESI): m/z [M+H]⁺ calc'd for C₄₄H₅₄N₄O₆Si 762.4; found 763.3.
Step 4. Ethyl [(Z)—N—[(Z)-(2M)-3-[3-[(tert-butyldiphenylsilyl)oxy]-2,2-dimethylpropyl]-1-ethyl-2-[2-[(1S)-1-methoxyethyl]pyridin-3-yl]indole-5-carbonyloxy] carbamimidoyl] formate (1.30 g, 1.7 mmol) was heated to 150° C. and stirred for 4 h, then purified by silica gel column chromatography to give ethyl 5-[(2M)-3-[3-[(tert-butyldiphenylsilyl)oxy]-2,2-dimethylpropyl]-1-ethyl-2-[2-[(1S)-1-methoxyethyl]pyridin-3-yl]indol-5-yl]-1,2,4-oxadiazole-3-carboxylate (600 mg, 28% yield) as a solid. LCMS (ESI): m/z [M+H]⁺ calc'd for C₄₄H₅₂N₄O₅Si 744.4; found 745.3.
Step 5. To a mixture of ethyl 5-[(2M)-3-[3-[(tert-butyldiphenylsilyl)oxy]-2,2-dimethylpropyl]-1-ethyl-2-[2-[(1S)-1-methoxyethyl]pyridin-3-yl]indol-5-yl]-1,2,4-oxadiazole-3-carboxylate (1.1 g, 1.5 mmol) in EtOH (6 mL) and THF (6 mL) at 0° C. was added NaBH₄ (112 mg, 3.0 mmol) in portions. The mixture was stirred at rt for 1 h, then the mixture was cooled to 0° C. and saturated NH₄Cl was added, and the mixture was extracted with EtOAc (30 mL). The organic layer was washed with brine (20 mL), dried over anhydrous Na₂SO₄, filtered and the filtrate was concentrated under reduced pressure to give [5-[(2M)-3-[3-[(tert-butyldiphenylsilyl)oxy]-2,2-dimethylpropyl]-1-ethyl-2-[2-[(1S)-1-methoxyethyl]pyridin-3-yl]indol-5-yl]-1,2,4-oxadiazol-3-yl]methanol (900 mg, 78% yield) as a solid. LCMS (ESI): m/z [M+H]⁺ calc'd for C₄₂H₅₀N₄O₄Si 702.4; found 703.4.
Step 6. To a mixture of [5-[(2M)-3-[3-[(tert-butyldiphenylsilyl)oxy]-2,2-dimethylpropyl]-1-ethyl-2-[2-[(1S)-1-methoxyethyl]pyridin-3-yl]indol-5-yl]-1,2,4-oxadiazol-3-yl]methanol (900 mg, 1.3 mmol) and Ph₃P (504 mg, 1.92 mmol) in DCM (9 mL) was added CBr₄ (637 mg, 1.92 mmol). The mixture was stirred at rt for 3 h, then H₂O was added, and the mixture was extracted with EtOAc (10 mL). The combined organic layers were washed with brine (10 mL), dried over anhydrous Na₂SO₄, and filtered. The filtrate was concentrated under reduced pressure and the residue was purified by silica gel column chromatography to give (2M)-5-[3-(bromomethyl)-1,2,4-oxadiazol-5-yl]-3-[3-[(tert-butyldiphenylsilyl)oxy]-2,2-dimethylpropyl]-1-ethyl-2-[2-[(1S)-1-methoxyethyl]pyridin-3-yl]indole (700 mg, 36% yield) as a solid. LCMS (ESI): m/z [M+H]⁺ calc'd for C₄₂H₄₉BrN₄O₃Si 764.3; found 765.2.
Step 7. To a mixture of (2M)-5-[3-(bromomethyl)-1,2,4-oxadiazol-5-yl]-3-[3-[(tert-butyldiphenylsilyl)oxy]-2,2-dimethylpropyl]-1-ethyl-2-[2-[(1S)-1-methoxyethyl]pyridin-3-yl]indole (1.0 g, 1.3 mmol) and tert-butyl 2-[(diphenylmethylidene)amino] acetate (579 mg, 2.0 mmol) in toluene (4.2 mL) and DCM (1.8 mL) at 0° C. was added KOH (7.0 g, 124.8 mmol) in H₂O (2 mL) and cinchonanium (158 mg, 0.26 mmol). The mixture was warmed to rt and stirred for 3 h, then H₂O was added, and the mixture was extracted with EtOAc (10 mL). The combined organic layers were washed with brine (5 mL), dried over anhydrous Na₂SO₄, and filtered. The filtrate was concentrated under reduced pressure and the residue was purified by silica gel column chromatography to give tert-butyl 3-[5-[(2M)-3-[3-[(tert-butyldiphenylsilyl)oxy]-2,2-dimethylpropyl]-1-ethyl-2-[2-[(1S)-1-methoxyethyl]pyridin-3-yl]indol-5-yl]-1,2,4-oxadiazol-3-yl]-2-[(diphenylmethylidene)amino]propanoate (350 mg, 25% yield) as a solid. LCMS (ESI): m/z [M+H]⁺ calc'd for C₆₁H₆₉N₅O₅Si 979.5; found 980.4.
Step 8. To a mixture of tert-butyl 3-[5-[(2M)-3-[3-[(tert-butyldiphenylsilyl)oxy]-2,2-dimethylpropyl]-1-ethyl-2-[2-[(1S)-1-methoxyethyl]pyridin-3-yl]indol-5-yl]-1,2,4-oxadiazol-3-yl]-2-[(diphenylmethylidene)amino]propanoate (1.80 g, 1.8 mmol) in DCM (18 mL) at 0° C. was added TFA (18 mL) dropwise. The mixture was warmed to rt and stirred for 2 h, then concentrated under reduced pressure to give 2-amino-3-[5-[(2M)-3-[3-[(tert-butyldiphenylsilyl)oxy]-2,2-dimethylpropyl]-1-ethyl-2-[2-[(1S)-1-methoxyethyl]pyridin-3-yl]indol-5-yl]-1,2,4-oxadiazol-3-yl]propanoic acid (4 g) as an oil. LCMS (ESI): m/z [M+H]⁺ calc'd for C₄₄H₅₃N₅O₅Si 759.4; found 760.2.
Step 9. To a mixture of 2-amino-3-[5-[(2M)-3-[3-[(tert-butyldiphenylsilyl)oxy]-2,2-dimethylpropyl]-1-ethyl-2-[2-[(1S)-1-methoxyethyl]pyridin-3-yl]indol-5-yl]-1,2,4-oxadiazol-3-yl]propanoic acid (4.0 g, 5.3 mmol) and NaHCO₃ (2.65 g, 30 mmol) in THF (20 mL) and H₂O (20 mL) was added Boc₂O (1.72 g, 7.9 mmol) dropwise. The mixture was stirred at rt for 2 h, then H₂O was added, and the mixture was extracted with EtOAc (3×50 mL). The combined organic layers were dried over anhydrous Na₂SO₄ and filtered. The filtrate was concentrated under reduced pressure and the residue was purified by silica gel column chromatography to give 2-[(tert-butoxycarbonyl)amino]-3-[5-[(2M)-3-[3-[(tert-butyldiphenylsilyl)oxy]-2,2-dimethylpropyl]-1-ethyl-2-[2-[(1S)-1-methoxyethyl]pyridin-3-yl]indol-5-yl]-1,2,4-oxadiazol-3-yl]propanoic acid (1.2 g, 21% yield) as a solid. LCMS (ESI): m/z [M+H]⁺ calc'd for C₄₉H₆₁N₅O₇Si 859.4; found 860.2.
Step 10. To a mixture of 2-[(tert-butoxycarbonyl)amino]-3-[5-[(2M)-3-[3-[(tert-butyldiphenylsilyl)oxy]-2,2-dimethylpropyl]-1-ethyl-2-[2-[(1S)-1-methoxyethyl]pyridin-3-yl]indol-5-yl]-1,2,4-oxadiazol-3-yl]propanoic acid (1.00 g, 1.2 mmol), methyl (3S)-1,2-diazinane-3-carboxylate (0.34 g, 2.3 mmol), HOBT (0.08 g, 0.6 mmol) and DIPEA (1.50 g, 11.6 mmol) in DCM (10 mL) at 0° C. under an atmosphere of N₂ was added EDCl (0.33 g, 1.7 mmol) in portions. The mixture was warmed to rt and stirred for 2 h, then H₂O (50 mL) was added, and the mixture was extracted with EtOAc (3×50 mL). The combined organic layers were dried over anhydrous Na₂SO₄ and filtered. The filtrate was concentrated under reduced pressure and the residue was purified by silica gel column chromatography to give methyl (3S)-1-[(2S)-2-[(tert-butoxycarbonyl)amino]-3-[5-(3-[3-[(tert-butyldiphenylsilyl)oxy]-2,2-dimethylpropyl]-1-ethyl-2-[2-[(1S)-1-methoxyethyl]pyridin-3-yl]indol-5-yl)-1,2,4-oxadiazol-3-yl]propanoyl]-1,2-diazinane-3-carboxylate (800 mg, 63% yield) as a solid. LCMS (ESI): m/z [M+H]⁺ calc'd for C₅₅H₇₁N₇O₈Si 985.5; found 986.6.
Step 11. To a mixture of methyl (3S)-1-[(2S)-2-[(tert-butoxycarbonyl)amino]-3-[5-(3-[3-[(tert-butyldiphenylsilyl)oxy]-2,2-dimethylpropyl]-1-ethyl-2-[2-[(1S)-1-methoxyethyl]pyridin-3-yl]indol-5-yl)-1,2,4-oxadiazol-3-yl]propanoyl]-1,2-diazinane-3-carboxylate (800 mg, 0.8 mmol) in THF (5 mL) at 0° C. under an atmosphere of N₂ was added 1M TBAF in THF (5 mL) dropwise. The mixture was heated to 60° C. and stirred overnight, then H₂O (100 mL) was added, and the mixture was extracted with EtOAc (3×50 mL). The combined organic layers were dried over anhydrous Na₂SO₄, filtered and the filtrate was concentrated under reduced pressure to give (3S)-1-[(2S)-2-[(tert-butoxycarbonyl)amino]-3-[5-[(2M)-1-ethyl-3-(3-hydroxy-2,2-dimethylpropyl)-2-[2-[(1S)-1-methoxyethyl]pyridin-3-yl]indol-5-yl]-1,2,4-oxadiazol-3-yl]propanoyl]-1,2-diazinane-3-carboxylic acid (680 mg) as a solid. LCMS (ESI): m/z [M+H]⁺ calc'd for C₃₈H₅₁N₇O₈ 733.3; found 734.3.
Step 12. To a mixture of (3S)-1-[(2S)-2-[(tert-butoxycarbonyl)amino]-3-[5-[(2M)-1-ethyl-3-(3-hydroxy-2,2-dimethylpropyl)-2-[2-[(1S)-1-methoxyethyl]pyridin-3-yl]indol-5-yl]-1,2,4-oxadiazol-3-yl]propanoyl]-1,2-diazinane-3-carboxylic acid (500 mg, 0.68 mmol), HOBT (460 mg, 3.4 mmol) and DIPEA (2.64 g, 20.4 mmol) in DCM (100 mL) at 0° C. under an atmosphere of N₂ was added EDCl (2.61 g, 13.6 mmol) in portions. The mixture was warmed to rt and stirred overnight, then concentrated under reduced pressure and the residue was purified by preparative-TLC to give tert-butyl ((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(5,3)-oxadiazola-1(5,3)-indola-6(1,3)- pyridazinacycloundecaphane-4-yl)carbamate (22 mg, 18% yield) as a solid. LCMS (ESI): m/z [M+H]⁺ calc'd for C₃₈H₄₉N₇O₇ 715.4; found 716.2.
Step 13. To a mixture of tert-butyl ((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(5,3)-oxadiazola-1(5,3)-indola-6(1,3)-pyridazinacycloundecaphane-4-yl)carbamate (20 mg, 0.03 mmol) in DCM (0.30 mL) at 0° C. under an atmosphere of N₂ was added TFA (0.1 mL) dropwise. The mixture was warmed to rt and stirred for 1 h, then concentrated under reduced pressure to give (6³S,4S,Z)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-2(5,3)-oxadiazola-1(5,3)-indola-6(1,3)- pyridazinacycloundecaphane-5,7-dione (30 mg) as an oil. LCMS (ESI): m/z [M+H]⁺ calc'd for C₃₃H₄₁N₇O₅ 615.3; found 616.4.
Step 14. To a mixture of (6³S,4S,Z)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-2(5,3)-oxadiazola-1(5,3)-indola-6(1,3)-pyridazinacycloundecaphane-5,7-dione (20 mg, 0.03 mmol), DIPEA (42 mg, 0.33 mmol) and (2S)-2-(1-[1-[4-(dimethylamino)-4-methylpent-2-ynoyl]-4-fluoropiperidin-4-yl]-N-methylformamido)-3-methylbutanoic acid (19 mg, 0.05 mmol) in DMF (1 mL) at 0° C. under an atmosphere of N₂ was added HATU (16 mg, 0.04 mmol) in portions. The mixture was warmed to rt and stirred for 1 h, then purified by preparative-HPLC to give 1-(4-(dimethylamino)-4-methylpent-2-ynoyl)-N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(5,3)-oxadiazola-1(5,3)-indola-6(1,3)- pyridazinacycloundecaphane-4-yl)amino)-3-methyl-1-oxobutan-2-yl)-4-fluoro-N-methylpiperidine-4-carboxamide (2.4 mg, 7% yield) as a solid. LCMS (ESI): m/z [M+H]⁺ calc'd for C₅₃H₇₁FN₁₀O₈ 994.5; found 995.4; ¹H NMR (400 MHZ, DMSO-d₆) δ 8.78 (dd, J=4.7, 1.7 Hz, 1H), 8.63 (s, 1H), 8.33 (s, 1H), 7.95-7.68 (m, 3H), 7.55 (dd, J=7.7, 4.7 Hz, 1H), 5.79 (s, 1H), 5.07 (d, J=11.7 Hz, 1H), 4.62 (d, J=10.3 Hz, 1H), 4.34-4.20 (m, 7H), 3.70-3.49 (m, 3H), 3.23 (s, 3H), 3.17-3.03 (m, 5H), 2.98-2.89 (m, 3H), 2.77 (t, J=12.2 Hz, 1H), 2.46-2.41 (m, 1H), 2.20 (dd, J=10.7, 6.6 Hz, 7H), 2.15-2.03 (m, 5H), 1.81 (d, J=12.5 Hz, 1H), 1.65 (d, J=13.0 Hz, 1H), 1.53 (d, J=11.9 Hz, 1H), 1.37 (t, J=6.3 Hz, 9H), 1.03-0.86 (m, 10H), 0.88-0.80 (m, 2H), 0.80-0.74 (m, 3H).

### Example 2. Synthesis of 1-(4-(dimethylamino)-4-methylpent-2-ynoyl)-N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(3,5)-oxadiazola-1(5,3)-indola-6(1,3)-pyridazinacycloundecaphane-4-yl)amino)-3-methyl-1-oxobutan-2-yl)-4-fluoro-N-methylpiperidine-4-carboxamide (A127)

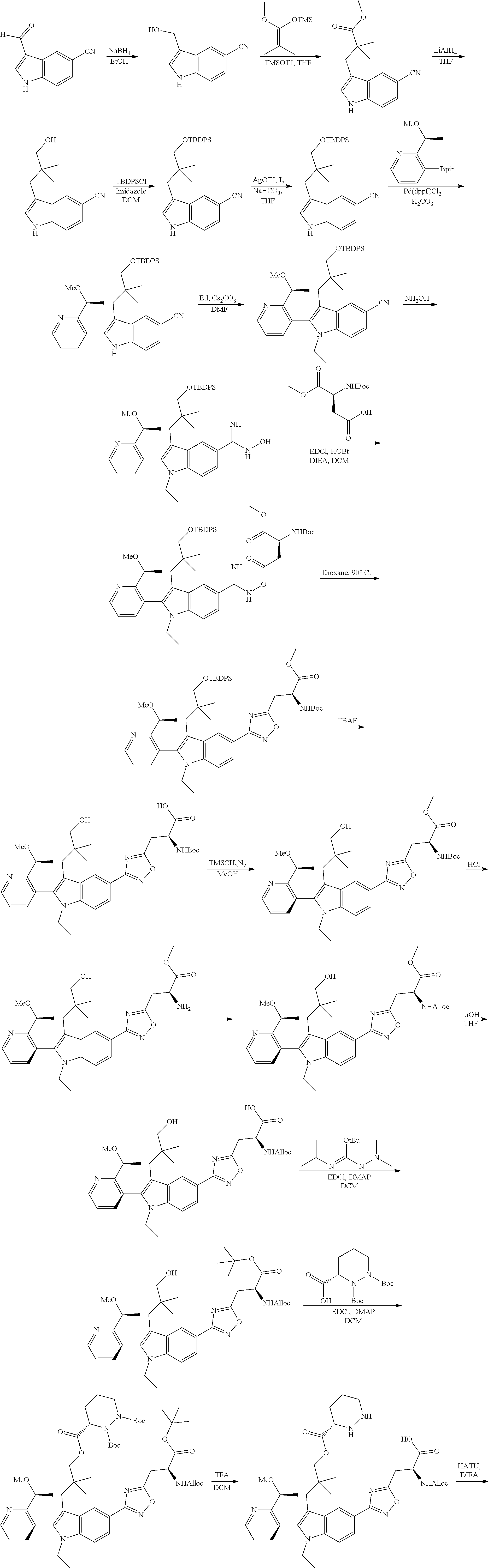

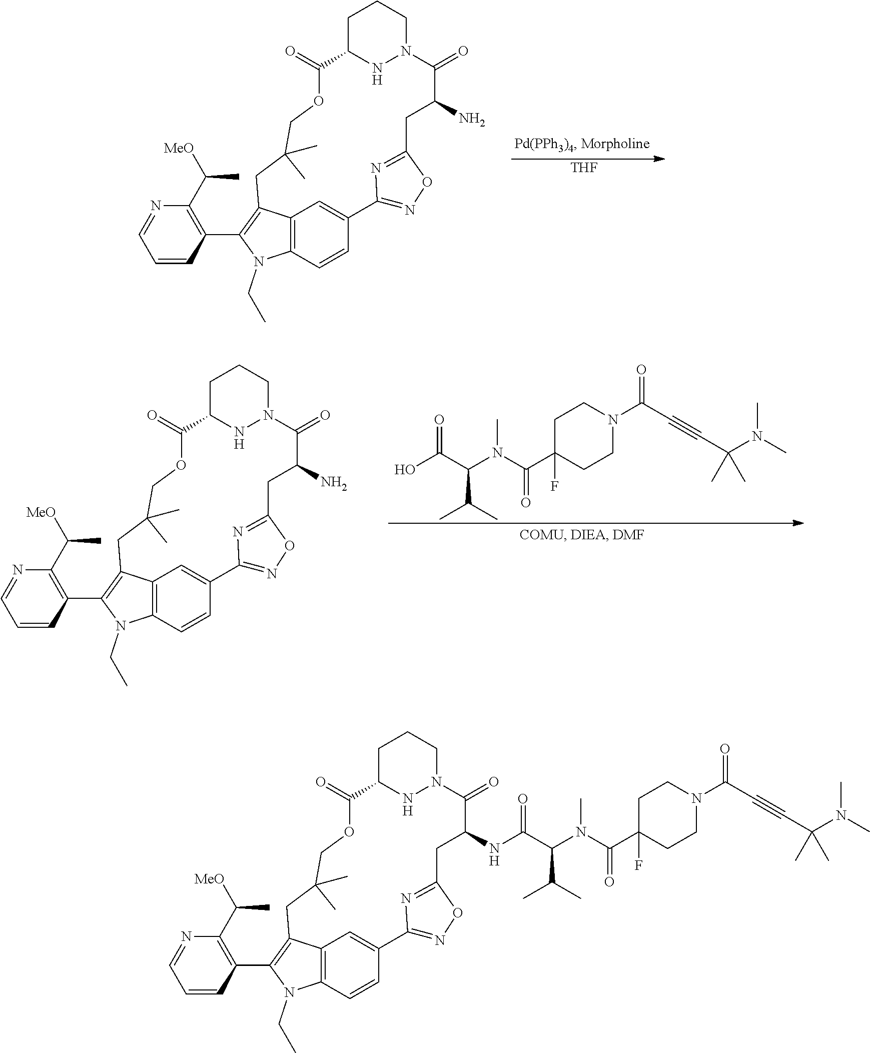

Step 1. To a mixture of 3-formyl-1H-indole-5-carbonitrile (24.8 g, 145.7 mmol) in EtOH (248 mL) at 0° C. was added NaBH₄ (8.05 g, 218.6 mmol) in portions. The mixture was stirred at 0° C. for 2 h then saturated NH₄Cl (500 mL) was added, and the volatiles were removed under reduced pressure. The mixture was extracted with DCM (3×200 mL) and the combined organic layers were washed with water (3×200 mL), dried over anhydrous Na₂SO₄, and filtered. Th filtrate was concentrated under reduced pressure and the residue was purified by silica gel column chromatography to give 3-(hydroxymethyl)-1H-indole-5-carbonitrile (21 g, 84% yield) as a solid. LCMS (ESI): m/z [M−H]⁺ calc'd for C₁₀H₈N₂O 172.1; found 171.1.
Step 2. To a mixture of 3-(hydroxymethyl)-1H-indole-5-carbonitrile (20.0 g, 116.2 mmol) in THF (200 mL) at −40° C. under an atmosphere of Ar was added [(1-methoxy-2-methylprop-1-en-1-yl)oxy]trimethylsilane (50.62 g, 290.4 mmol) and TMSOTf (19.36 g, 87.1 mmol) dropwise. The mixture was stirred at −40° C. for 2 h, then brine (200 mL) was added at 0° C. The aqueous and organic layers were partitioned, and the organic layer was extracted with EtOAc (3×200 mL). The combined organic layers were concentrated under reduced pressure and the residue was purified by silica gel column chromatography to give methyl 3-(5-cyano-1H-indol-3-yl)-2,2-dimethylpropanoate (22 g, 74% yield) as a solid. LCMS (ESI): m/z [M−H]⁺ calc'd for C₁₅H₁₆N₂O₂ 256.1; found 255.1.
Step 3. To mixture of methyl 3-(5-cyano-1H-indol-3-yl)-2,2-dimethylpropanoate (22 g, 85.8 mmol) in THF (220 mL) at 0° C. was added 1M LiAlH₄ in THF (171.7 mL, 171.7 mmol) dropwise. The mixture was stirred at 0° C. for 2 h, then Na₂SO₄·10H₂O was added, the mixture was filtered, and the filter cake was washed with DCM (3×300 mL). The filtrate was concentrated under reduced pressure to give 3-(3-hydroxy-2,2-dimethylpropyl)-1H-indole-5-carbonitrile (12.8 g, 65% yield) as a solid. LCMS (ESI): m/z [M−H]⁺ calc'd for C₁₄H₁₆N₂O 228.1; found 255.1.
Step 4. To a mixture of 3-(3-hydroxy-2,2-dimethylpropyl)-1H-indole-5-carbonitrile (15.0 g, 65.7 mmol) in DCM (150 mL) at 0° C. was added imidazole (11.18 g, 164.3 mmol) and TBDPSCl (23.48 g, 85.4 mmol). The mixture was warmed to rt and stirred overnight, then concentrated under reduced pressure and the residue was purified by silica gel column chromatography to give 3-[3-[(tert-butyldiphenylsilyl)oxy]-2,2-dimethylpropyl]-1H-indole-5-carbonitrile (30 g, 97% yield) as an oil. LCMS (ESI): m/z [M−H]⁺ calc'd for C₃₀H₃₄N₂OSi 466.2; found 465.2.
Step 5. To a mixture of 3-[3-[(tert-butyldiphenylsilyl)oxy]-2,2-dimethylpropyl]-1H-indole-5-carbonitrile (18.0 g, 38.6 mmol) in THF (180 mL) at 0° C. under an atmosphere of N₂ was added NaHCO₃ (3.89 g, 46.3 mmol), AgOTf (10.9 g, 42.4 mmol) and I₂ (8.81 g, 34.7 mmol). The mixture was stirred at 0° C. for 2 h, then 5% aqueous Na₂S₂O₃ was added, and the mixture was extracted with EtOAc (3×200 mL). The combined organic layers were washed with water (3×200 mL), dried over anhydrous Na₂SO₄, and filtered. The filtrate was concentrated under reduced pressure and the residue was purified by silica gel column chromatography to give 3-[3-[(tert-butyldiphenylsilyl)oxy]-2,2-dimethylpropyl]-2-iodo-1H-indole-5-carbonitrile (18.2 g, 80% yield) as a solid. LCMS (ESI): m/z [M+Na]⁺ calc'd for C₃₀H₃₃IN₂NaOSi 615.1; found 615.0.
Step 6. To a mixture of 3-[3-[(tert-butyldiphenylsilyl)oxy]-2,2-dimethylpropyl]-2-iodo-1H-indole-5-carbonitrile (18.2 g, 30.7 mmol) and 2-[(1S)-1-methoxyethyl]-3-(4,4,5,5-tetramethyl-1,3,2-dioxaborolan-2-yl)pyridine (32.33 g, 122.9 mmol) in 1,4-dioxane (150 mL) and H₂O (30 mL) under an atmosphere of Ar was added K₂CO₃ (10.60 g, 76.8 mmol), Pd(dppf)Cl₂ (4.49 g, 6.1 mmol). The mixture was heated to 50° C. and stirred for 3 h, then concentrated under reduced pressure and the residue was purified by silica gel column chromatography to give 3-[3-[(tert-butyldiphenylsilyl)oxy]-2,2-dimethylpropyl]-2-[2-[(1S)-1-methoxyethyl]pyridin-3-yl]-1H-indole-5-carbonitrile (20 g) as an oil. LCMS (ESI): m/z [M+H]⁺ calc'd for C₃₈H₄₃N₃O₂Si 601.3; found 602.3.
Step 7. To a mixture of 3-[3-[(tert-butyldiphenylsilyl)oxy]-2,2-dimethylpropyl]-2-[2-[(1S)-1-methoxyethyl]pyridin-3-yl]-1H-indole-5-carbonitrile (22.0 g, 36.6 mmol) in DMF (220 mL) at 0° C. was added Cs₂CO₃ (35.73 g, 109.7 mmol) and Etl (34.21 g, 219.3 mmol). The mixture was stirred at 0° C. for 2 h, then H₂O was added, and the mixture extracted with EtOAc (300 mL). The organic layer was washed with H₂O (3×300 mL), dried over anhydrous Na₂SO₄, and filtered. The filtrate was concentrated under reduced pressure and the residue was purified by silica gel column chromatography to give 3-[3-[(tert-butyldiphenylsilyl)oxy]-2,2-dimethylpropyl]-1-ethyl-2-[2-[(1S)-1-methoxyethyl]pyridin-3-yl]indole-5-carbonitrile (15.6 g, 63% yield) as an oil. LCMS (ESI): m/z [M+H]⁺ calc'd for C₄₀H₄₇N₃O₂Si 629.3; found 630.0.
Step 8. To a mixture of 3-[3-[(tert-butyldiphenylsilyl)oxy]-2,2-dimethylpropyl]-1-ethyl-2-[2-[(1S)-1-methoxyethyl]pyridin-3-yl]indole-5-carbonitrile (15.60 g, 24.8 mmol) in MeOH (156 mL) was added NH₂OH, 50% in H₂O (9.81 g, 296.9 mmol). The mixture was heated to 50° C. and stirred for 3 h, then concentrated under reduced pressure and the residue was purified by silica gel column chromatography to give 3-[3-[(tert-butyldiphenylsilyl)oxy]-2,2-dimethylpropyl]-1-ethyl-N-hydroxy-2-[2-[(1S)-1-methoxyethyl]pyridin-3-yl]indole-5-carboximidamide (14.6 g, 89% yield) as an oil. LCMS (ESI): m/z [M+H]⁺ calc'd for C₄₀H₅₀N₄O₃Si 662.4; found 663.2.
Step 9. To a mixture of 3-[3-[(tert-butyldiphenylsilyl)oxy]-2,2-dimethylpropyl]-1-ethyl-N-hydroxy-2-[2-[(1S)-1-methoxyethyl]pyridin-3-yl]indole-5-carboximidamide (14.60 g, 22.0 mmol) in DCM (146 mL) at −5° C. was added DIPEA (14.23 g, 110.1 mmol), HOBt (0.60 g, 4.4 mmol), followed by EDC·HCl (5.07 g, 26.4 mmol) in portions over 2 min. The mixture was allowed to warm to rt and stirred for 2 h, then concentrated under reduced pressure and the residue was purified by silica gel column chromatography to give 4-(3-[3-[(tert-butyldiphenylsilyl)oxy]-2,2-dimethylpropyl]-1-ethyl-2-[2-[(1S)-1-methoxyethyl]pyridin-3-yl]indol-5-yl) methanimidamido 1-methyl (2S)-2-[(tert-butoxycarbonyl)amino]butanedioate (18.1 g, 92% yield) as an oil. LCMS (ESI): m/z [M+H]⁺ calc'd for C₅₀H₆₅N₅O₈Si 891.5; found 892.3.
Step 10. A mixture of 4-(3-[3-[(tert-butyldiphenylsilyl)oxy]-2,2-dimethylpropyl]-1-ethyl-2-[2-[(1S)-1-methoxyethyl]pyridin-3-yl]indol-5-yl) methanimidamido 1-methyl (2S)-2-[(tert-butoxycarbonyl)amino]butanedioate (18 g, 20.2 mmol) in 1,4-dioxane (900 mL) was heated to 90° C. and stirred for 3 h. The mixture was concentrated under reduced pressure and the residue was purified by silica gel column chromatography to give methyl 2-[(tert-butoxycarbonyl)amino]-3-[3-(3-{3-[(tert-butyldiphenylsilyl)oxy]-2,2-dimethylpropyl}-1-ethyl-2-{2-[(1S)-1-methoxyethyl]pyridin-3-yl}indol-5-yl)-1,2,4-oxadiazol-5-yl]propanoate (16.5 g, 94% yield) as an oil. LCMS (ESI): m/z [M+H]⁺ calc'd for C₅₀H₆₃N₅O₇Si 873.4; found 874.4.
Step 11. To a mixture of methyl 2-[(tert-butoxycarbonyl)amino]-3-[3-(3-{3-[(tert-butyldiphenylsilyl)oxy]-2,2-dimethylpropyl}-1-ethyl-2-{2-[(1S)-1-methoxyethyl]pyridin-3-yl}indol-5-yl)-1,2,4-oxadiazol-5-yl]propanoate (18 g, 20.6 mmol) in THF (180 mL) was added 1M TBAF in THF (180 mL) dropwise. The mixture was heated to 60° C. and stirred overnight, then H₂O was added, and the mixture extracted with DCM (3×300 mL). The combined organic layers were washed with brine (6×300 mL), dried over anhydrous Na₂SO₄, filtered and the filtrate was concentrated under reduced pressure to give 2-[(tert-butoxycarbonyl)amino]-3-{3-[(2M)-1-ethyl-3-(3-hydroxy-2,2-dimethylpropyl)-2-{2-[(1S)-1-methoxyethyl]pyridin-3-yl}indol-5-yl]-1,2,4-oxadiazol-5-yl}propanoic acid (14 g) as an oil. LCMS (ESI): m/z [M−H]⁺ calc'd for C₃₃H₄₃N₅O₇ 621.3; found 620.3.
Step 12. To a mixture of 2-[(tert-butoxycarbonyl)amino]-3-{3-[(2M)-1-ethyl-3-(3-hydroxy-2,2-dimethylpropyl)-2-{2-[(1S)-1-methoxyethyl]pyridin-3-yl}indol-5-yl]-1,2,4-oxadiazol-5-yl}propanoic acid (14 g, 22.5 mmol) in MeOH (140 mL) at 0° C. was added TMSCHN₂ (12.86 g, 112.6 mmol). The mixture was stirred at 0° C. for 2 h, then concentrated under reduced pressure and the residue was purified by silica gel column chromatography to give methyl (2R)-2-[(tert-butoxycarbonyl)amino]-3-{3-[(2M)-1-ethyl-3-(3-hydroxy-2,2-dimethylpropyl)-2-{2-[(1S)-1-methoxyethyl]pyridin-3-yl}indol-5-yl]-1,2,4-oxadiazol-5-yl}propanoate (3.5 g, 25% yield) as an oil. LCMS (ESI): m/z [M+H]⁺ calc'd for C₃₄H₄₅N₅O₇ 635.3; found 636.4.
Step 13. To a mixture of methyl (2R)-2-[(tert-butoxycarbonyl)amino]-3-{3-[(2M)-1-ethyl-3-(3-hydroxy-2,2-dimethylpropyl)-2-{2-[(1S)-1-methoxyethyl]pyridin-3-yl}indol-5-yl]-1,2,4-oxadiazol-5-yl}propanoate (2.0 g, 3.2 mmol) in 1,4-dioxane (20 mL) was added HCl in 1,4-dioxane (20 mL). The mixture was stirred at rt for 1 h, then concentrated under reduced pressure to give methyl (2R)-2-amino-3-{3-[(2M)-1-ethyl-3-(3-hydroxy-2,2-dimethylpropyl)-2-{2-[(1S)-1-methoxyethyl]pyridin-3-yl}indol-5-yl]-1,2,4-oxadiazol-5-yl}propanoate (1.5 g, 89% yield) as a solid. LCMS (ESI): m/z [M+H]⁺ calc'd for C₂₉H₃₇N₅O₅ 535.3; found 536.4.
Step 14. To a mixture of methyl (2R)-2-amino-3-{3-[(2M)-1-ethyl-3-(3-hydroxy-2,2-dimethylpropyl)-2-{2-[(1S)-1-methoxyethyl]pyridin-3-yl}indol-5-yl]-1,2,4-oxadiazol-5-yl}propanoate (3.0 g, 5.6 mmol) in THF (30 mL) and H₂O (6 mL) at 0° C. was added NaHCO₃ (1.18 g, 14.0 mmol) and allyl chlorocarbonate (1.01 g, 8.4 mmol). The mixture was stirred at 0° C. for 2 h, then concentrated under reduced pressure and the residue was purified by silica gel column chromatography to give methyl 3-{3-[(2M)-1-ethyl-3-(3-hydroxy-2,2-dimethylpropyl)-2-{2-[(1S)-1-methoxyethyl]pyridin-3-yl}indol-5-yl]-1,2,4-oxadiazol-5-yl}-2-{[(prop-2-en-1-yloxy)carbonyl]amino}propanoate (1.5 g, 43% yield) as a solid. LCMS (ESI): m/z [M+H]⁺ calc'd for C₃₃H₄₁N₅O₇ 619.3; found 620.4.
Step 15. To a mixture of methyl 3-{3-[(2M)-1-ethyl-3-(3-hydroxy-2,2-dimethylpropyl)-2-{2-[(1S)-1-methoxyethyl]pyridin-3-yl}indol-5-yl]-1,2,4-oxadiazol-5-yl}-2-{[(prop-2-en-1-yloxy)carbonyl]amino}propanoate (1.5 g, 2.1 mmol) in THF (15 mL) at 0° C. was added LiOH (16 mg, 6.8 mmol) in H₂O (15 mL). The mixture was stirred at 0° C. for 1 h, then acidified to PH ˜4 with aqueous HCl and extracted with DCM (3×30 mL). The combined organic layers were washed with brine (3×30 mL), dried over anhydrous Na₂SO₄, filtered and the filtrate was concentrated under reduced pressure to give (2R)-3-[3-[(2M)-1-ethyl-3-(3-hydroxy-2,2-dimethylpropyl)-2-[2-[(1S)-1-methoxyethyl]pyridin-3-yl]indol-5-yl]-1,2,4-oxadiazol-5-yl]-2-[(prop-2-en-1-yloxy)carbonyl]amino]propanoic acid (1.46 g) as a solid. LCMS (ESI): m/z [M+H]⁺ calc'd for C₃₂H₃₉N₅O₇ 605.3; found 606.3.
Step 16. To a mixture of (2R)-3-[3-[(2M)-1-ethyl-3-(3-hydroxy-2,2-dimethylpropyl)-2-[2-[(1S)-1-methoxyethyl]pyridin-3-yl]indol-5-yl]-1,2,4-oxadiazol-5-yl]-2-[(prop-2-en-1-yloxy)carbonyl]amino]propanoic acid (1.46 g, 2.4 mmol) in DCM (15 mL) at 0° C. was added (Z)—N,N-diisopropyltert-butoxymethanimidamide (2.41 g, 12.1 mmol). The mixture was heated to 40° C. and stirred overnight, then H₂O was added, and the mixture extracted with DCM (3×20 mL). The combined organic layers were washed with aqueous NH₄Cl (3×40 mL), dried over anhydrous Na₂SO₄, filtered and the filtrate was concentrated under reduced pressure to give tert-butyl 3-{3-[(2M)-1-ethyl-3-(3-hydroxy-2,2-dimethylpropyl)-2-{2-[(1S)-1-methoxyethyl]pyridin-3-yl}indol-5-yl]-1,2,4-oxadiazol-5-yl}-2-{[(prop-2-en-1-yloxy)carbonyl]amino}propanoate (2.3 g) as a solid. LCMS (ESI): m/z [M+H]⁺ calc'd for C₃₆H₄₇N₅O₇ 661.4; found 662.4.
Step 17. To a mixture of tert-butyl 3-{3-[(2M)-1-ethyl-3-(3-hydroxy-2,2-dimethylpropyl)-2-{2-[(1S)-1-methoxyethyl]pyridin-3-yl}indol-5-yl]-1,2,4-oxadiazol-5-yl}-2-{[(prop-2-en-1-yloxy)carbonyl]amino}propanoate (2.30 g, 3.5 mmol) in DCM (23 mL) at −5° C. was added DMAP (85 mg, 0.7 mmol), (3S)-1,2-bis (tert-butoxycarbonyl)-1,2-diazinane-3-carboxylic acid (3.44 g, 10.4 mmol) and EDCl (0.87 g, 4.5 mmol) in portions. The mixture was warmed to rt and stirred for 2 h, then concentrated under reduced pressure and the residue was purified by silica gel column chromatography to give 3-(2-[(2M)-5-[5-[(2R)-3-(tert-butoxy)-3-oxo-2-[[(prop-2-en-1-yloxy)carbonyl]amino]propyl]-1,2,4-oxadiazol-3-yl]-1-ethyl-2-[2-[(1S)-1-methoxyethyl]pyridin-3-yl]indol-3-yl]methyl]-2-methylpropyl) 1,2-di-tert-butyl (3S)-1,2-diazinane-1,2,3-tricarboxylate (2.29 g, 68% yield) as a solid. LCMS (ESI): m/z [M+H]⁺ calc'd for C₅₁H₇₁N₇O₁₂ 973.5; found 974.4.
Step 18. To a mixture of 3-(2-[(2M)-5-[5-[(2R)-3-(tert-butoxy)-3-oxo-2-[(prop-2-en-1-yloxy)carbonyl]amino]propyl]-1,2,4-oxadiazol-3-yl]-1-ethyl-2-[2-[(1S)-1-methoxyethyl]pyridin-3-yl]indol-3-yl]methyl]-2-methylpropyl) 1,2-di-tert-butyl (3S)-1,2-diazinane-1,2,3-tricarboxylate (2.29 g, 2.4 mmol) in DCM (30 mL) at 0° C. was added TFA (10 mL) dropwise. The mixture was stirred at 0° C. for 5 h, then concentrated under reduced pressure. The mixture was basified to PH ˜7 with saturated NaHCO₃ and extracted with DCM (3×300 mL). The combined organic layers were washed with H₂O (3×60 mL), dried over anhydrous Na₂SO₄, filtered and the filtrate was concentrated under reduced pressure to give 3-{3-[(2M)-3-{3-[(3S)-1,2-diazinane-3-carbonyloxy]-2,2-dimethylpropyl}-1-ethyl-2-{2-[(1S)-1-methoxyethyl]pyridin-3-yl}indol-5-yl]-1,2,4-oxadiazol-5-yl}-2-{[(prop-2-en-1-yloxy)carbonyl]amino}propanoic acid (1.4 g, 83% yield) as a solid. LCMS (ESI): m/z [M−H]⁺ calc'd for C₃₇H₄₇N₇O₈ 717.4; found 716.5.
Step 19. To a mixture of 3-{3-[(2M)-3-{3-[(3S)-1,2-diazinane-3-carbonyloxy]-2,2-dimethylpropyl}-1-ethyl-2-{2-[(1S)-1-methoxyethyl]pyridin-3-yl}indol-5-yl]-1,2,4-oxadiazol-5-yl}-2-{[(prop-2-en-1-yloxy)carbonyl]amino}propanoic acid (720 mg, 1.0 mmol) in DCM (7.2 mL) at 0° C. was added DIPEA (3.89 g, 30.1 mmol) and HATU (4.58 g, 12.0 mmol). The mixture was warmed to rt and stirred overnight, then concentrated under reduced pressure and the residue was purified by silica gel column chromatography to give prop-2-en-1-yl N-[(7S,13S,19M)-21-ethyl-20-[2-[(1S)-1-methoxyethyl]pyridin-3-yl]-17, 17-dimethyl-8,14-dioxo-4, 15-dioxa-3,9,21,27,28-pentaazapentacyclo[17.5.2.1\hat{ }[2,5].1\hat{ }[9,13].0\hat{ }[22,26]octacosa-1(25),2,5(28),19,22(26),23-hexaen-7-yl]carbamate (230 mg, 33% yield) as a solid. LCMS (ESI): m/z [M−H]⁺ calc'd for C₃₇H₄₅N₇O₇ 699.3; found 699.9.
Step 20. To a mixture of allyl ((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(3,5)-oxadiazola-1(5,3)-indola-6(1,3)-pyridazinacycloundecaphane-4-yl)carbamate (135 mg, 0.19 mmol) in THF (1.35 mL) under an atmosphere of Ar was added morpholine (50 mg, 0.58 mmol) and Pd(PPh₃)₄ (22.29 mg, 0.019 mmol). The mixture was heated to 35° C. and stirred for 4 h, then directly purified by silica gel column chromatography to give (6³S,4S,Z)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-2(3,5)-oxadiazola-1(5,3)-indola-6(1,3)-pyridazinacycloundecaphane-5,7-dione (120 mg) as a solid. LCMS (ESI): m/z [M+H]⁺ calc'd for C₃₃H₄₁N₇O₅ 615.3; found 616.4.
Step 21. To a mixture of (6³S,4S,Z)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-2(3,5)-oxadiazola-1(5,3)-indola-6(1,3)-pyridazinacycloundecaphane-5,7-dione (100 mg, 0.16 mmol) in DMF (1 mL) at 0° C. was added DIPEA (315 mg, 2.44 mmol), (2S)-2-(1-[1-[4-(dimethylamino)-4-methylpent-2-ynoyl]-4-fluoropiperidin-4-yl]-N-methylformamido)-3-methylbutanoic acid (129 mg, 0.32 mmol) and COMU (104 mg, 0.24 mmol). The mixture was stirred at 0° C. for 1 h, then purified by preparative-HPLC 1-(4-(dimethylamino)-4-methylpent-2-ynoyl)-N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(3,5)-oxadiazola-1(5,3)-indola-6(1,3)-pyridazinacycloundecaphane-4-yl)amino)-3-methyl-1-oxobutan-2-yl)-4-fluoro-N-methylpiperidine-4-carboxamide (25 mg, 15% yield) as a solid. LCMS (ESI): m/z [M−H]⁺ calc'd for C₅₃H₇₁ FN₁₀O₈ 994.5; found 995.8; ¹H NMR (400 MHZ, DMSO-d₆) δ 8.78 (dd, J=4.8, 1.8 Hz, 1H), 8.45 (d, J=17.0 Hz, 2H), 7.86-7.75 (m, 2H), 7.71 (d, J=8.7 Hz, 1H), 7.54 (dd, J=7.7, 4.7 Hz, 1H), 5.69 (s, 1H), 5.16 (d, J=11.8 Hz, 1H), 4.71-4.49 (m, 1H), 4.41-4.06 (m, 7H), 3.68-3.47 (m, 3H), 3.23 (s, 4H), 3.15-3.05 (m, 3H), 2.94 (d, J=11.1 Hz, 2H), 2.79-2.61 (m, 1H), 2.45-2.37 (m, 1H), 2.26-1.95 (m, 12H), 1.85-1.63 (m, 2H), 1.57-1.42 (m, 1H), 1.39-1.24 (m, 9H), 1.03-0.71 (m, 12H), 0.34 (s, 3H).

### Example 3. Synthesis of (4aR,7aS)-4-acryloyl-N-((2S)-1-(((2³R,6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-piperidinacycloundecaphane-4-yl)amino)-3-methyl-1-oxobutan-2-yl)-N-methylhexahydropyrrolo[3,4-b][1,4]oxazine-6(2H)-carboxamide (A12)

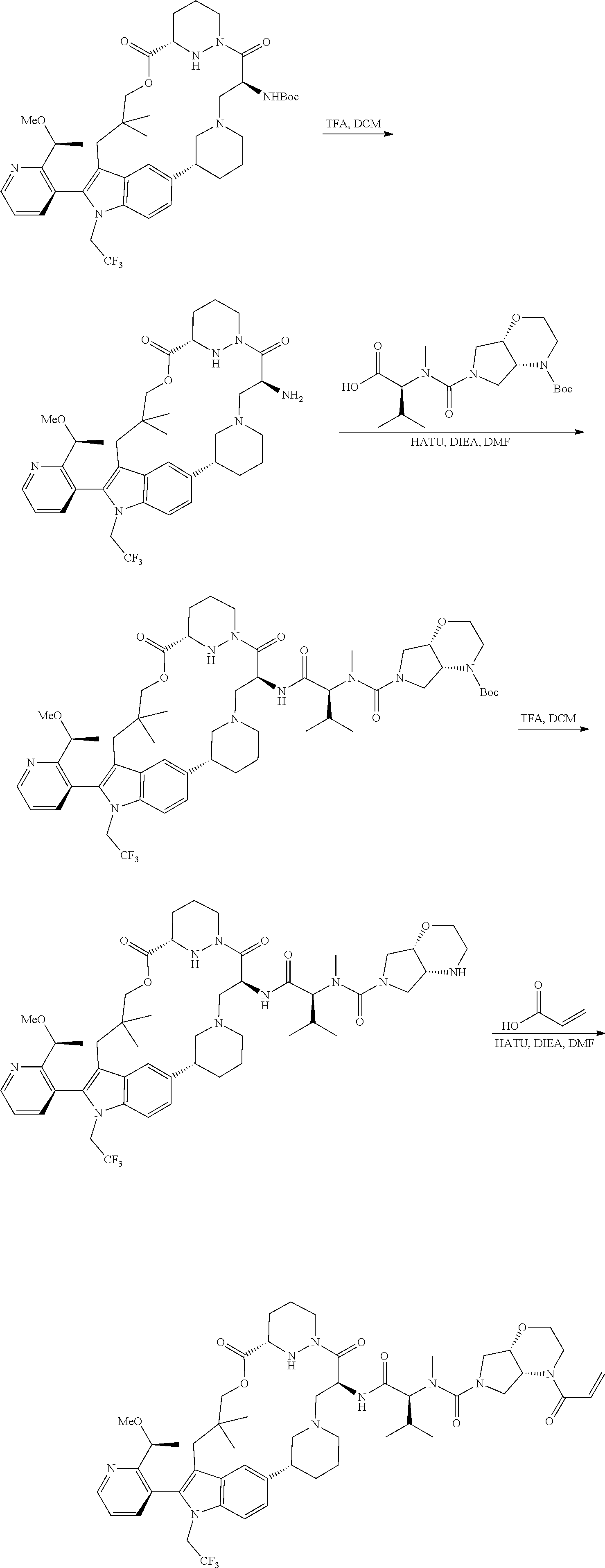

Step 1. To a mixture of tert-butyl ((6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-piperidinacycloundecaphane-4-yl)carbamate (Intermediate 15) (170 mg, 0.21 mmol) in DCM (2 mL) at 0° C. under an atmosphere of N₂ was added TFA (0.6 mL). The mixture was stirred at 0° C. for 2 h, then acidified to PH ˜8 with saturated aqueous NaHCO₃ and extracted with DCM (3×10 mL). The combined organic layers were washed with brine (3×10 mL), dried over anhydrous Na₂SO₄, filtered and the filtrate was concentrated under reduced pressure to give (2³R,6³S,4S)-4-amino-1²-(2-((S)-1-methoxyethyl)pyridin-3-yl)-10,10-dimethyl-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-piperidinacycloundecaphane-5,7-dione (160 mg) as an oil. LCMS (ESI): m/z [M+H]⁺ calc'd for C₃₆H₄₇F₃N₆O₄ 684.4; found 685.4.
Step 2. To mixture of (2³R,6³S,4S)-4-amino-1²-(2-((S)-1-methoxyethyl)pyridin-3-yl)-10,10-dimethyl-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-piperidinacycloundecaphane-5,7-dione (150 mg, 0.22 mmol) in DMF (2 mL) at 0° C. under an atmosphere of N₂ was added DIPEA (283 mg, 2.2 mmol), (2S)-2-[(4aR,7aS)-4-(tert-butoxycarbonyl)-hexahydropyrrolo[3,4-b][1,4]oxazine-6-carbonyl(methyl)amino]-3-methylbutanoic acid (127 mg, 0.33 mmol) and HATU (100 mg, 0.26 mmol) in portions. The mixture was warmed to rt and stirred for 2 h, then H₂O was added, and the mixture was extracted with EtOAc (3×10 mL). The combined organic layers were washed with brine (3×10 mL), dried over anhydrous Na₂SO₄, and filtered. The filtrate was concentrated under reduced pressure and the residue was purified by preparative-TLC to give tert-butyl (4aR,7aS)-6-(((2S)-1-(((2³R,6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-piperidinacycloundecaphane-4-yl)amino)-3-methyl-1-oxobutan-2-yl)(methyl)carbamoyl)hexahydropyrrolo[3,4-b][1,4]oxazine-4(4aH)-carboxylate (150 mg, 52% yield) as a solid. LCMS (ESI): m/z [M+H]⁺ calc'd for C₅₄H₇₆F₃N₉O₉ 1051.6; found 1052.5.
Step 3. To mixture of tert-butyl (4aR,7aS)-6-(((2S)-1-(((2³R,6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-piperidinacycloundecaphane-4-yl)amino)-3-methyl-1-oxobutan-2-yl)(methyl)carbamoyl)hexahydropyrrolo[3,4-b][1,4]oxazine-4(4aH)-carboxylate (150 mg, 0.14 mmol) in DCM (2 mL) at 0° C. under an atmosphere of N₂ was added TFA (0.70 mL). The mixture was warmed to rt and stirred for 2 h, then acidified to PH ˜8 with saturated NaHCO₃ and the mixture extracted with DCM (3×10 mL). The combined organic layers were washed with brine (3×10 mL), dried over anhydrous Na₂SO₄, filtered and the filtrate was concentrated under reduced pressure to give (4aR,7aS)—N-((2S)-1-(((2³R,6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-piperidinacycloundecaphane-4-yl)amino)-3-methyl-1-oxobutan-2-yl)-N-methylhexahydropyrrolo[3,4-b][1,4]oxazine-6(2H)-carboxamide (130 mg) as an oil. LCMS (ESI): m/z [M+H]⁺ calc'd for C₄₉H₆₈F₃N₉O₇ 951.5; found 952.6.
Step 4. To a mixture of (4aR,7aS)—N-((2S)-1-(((2³R,6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)- pyridazina-2(3,1)-piperidinacycloundecaphane-4-yl)amino)-3-methyl-1-oxobutan-2-yl)-N-methylhexahydropyrrolo[3,4-b][1,4]oxazine-6(2H)-carboxamide (120 mg, 0.13 mmol) in DMF (2 mL) at 0° C. under an atmosphere of N₂ was added DIPEA (163 mg, 1.26 mmol), acrylic acid (13.6 mg, 0.19 mmol) and HATU (57.5 mg, 0.15 mmol) in portions. The mixture was allowed to warm to rt and stirred for 2 h, then H₂O was added, and the mixture was extracted with EtOAc (3×10 mL). The combined organic layers were washed with brine (3×10 mL), dried over anhydrous Na₂SO₄, and filtered. The filtrate was concentrated under reduced pressure and the residue was purified by preparative-HPLC to give (4aR,7aS)-4-acryloyl-N-((2S)-1-(((2³R,6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-piperidinacycloundecaphane-4-yl)amino)-3-methyl-1-oxobutan-2-yl)-N-methylhexahydropyrrolo[3,4-b][1,4]oxazine-6(2H)-carboxamide (16 mg, 12% yield) as a solid. LCMS (ESI): m/z [M+H]⁺ calc'd for C₅₂H₇₀F₃N₉O₈ 1005.5; found 1006.9; ¹H NMR (300 MHZ, DMSO-d₆) δ 8.83 (dd, J=4.7, 1.7 Hz, 1H), 7.84 (t, J=7.4 Hz, 2H), 7.71 (d, J=8.5 Hz, 1H), 7.60 (dd, J=7.8, 4.7 Hz, 1H), 7.40 (s, 1H), 7.24 (d, J=8.5 Hz, 1H), 6.94-6.79 (m, 1H), 6.25 (d, J=16.7 Hz, 1H), 5.87-5.77 (m, 2H), 5.77 (s, 1H), 5.59-5.42 (m, 1H), 5.34 (d, J=12.0 Hz, 1H), 4.83 (s, 2H), 4.31 (d, J=12.9 Hz, 1H), 4.22 (d, J=6.8 Hz, 1H), 4.10-4.01 (m, 2H), 3.93 (d, J=11.3 Hz, 5H), 3.82-3.62 (m, 4H), 3.67-3.56 (m, 4H), 3.59-3.44 (m, 5H), 3.44-3.31 (m, 1H), 3.23 (d, J=5.7 Hz, 4H), 3.09 (s, 1H), 2.88-2.69 (m, 7H), 2.73-2.59 (m, 3H), 2.35 (m, 2H), 2.29 (s, 1H), 2.12 (s, 4H), 2.06 (s, 1H), 1.84 (s, 1H), 1.74-1.56 (m, 4H), 1.45 (d, J=6.1 Hz, 3H), 1.35-1.04 (m, 1H), 1.05-0.91 (m, 2H), 0.92-0.63 (m, 8H), 0.43 (s, 3H).

### Example 4. Synthesis of (4a R,7aS)-4-acryloyl-N-((2S)-1-(((2³S,6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-piperidinacycloundecaphane-4-yl)amino)-3-methyl-1-oxobutan-2-yl)-N-methylhexahydropyrrolo[3,4-b][1,4]oxazine-6(2H)-carboxamide (A26)

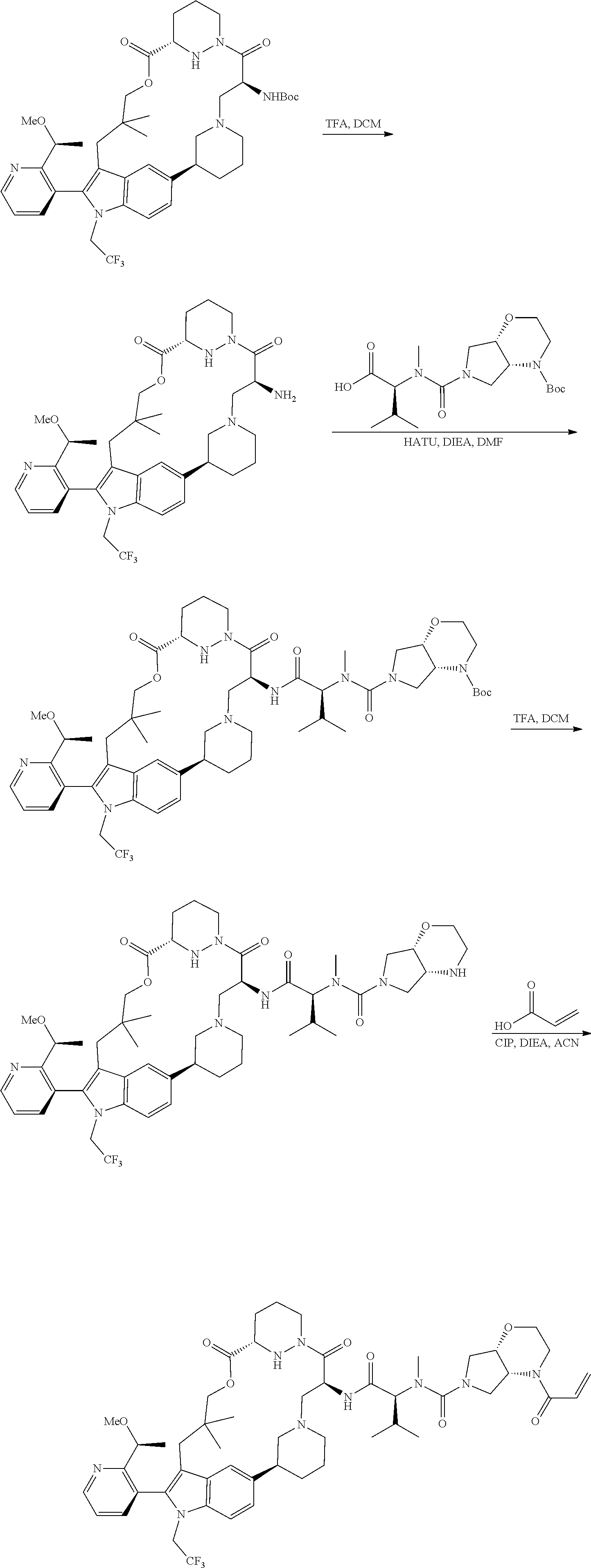

Step 1. To a mixture of tert-butyl ((2³S,6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-piperidinacycloundecaphane-4-yl)carbamate (200 mg, 0.26 mmol) in DCM (2 mL) at 0° C. was added TFA (0.7 mL). The mixture was stirred at 0° C. for 2 h, then acidified to PH ˜8 with saturated NaHCO₃ and extracted with DCM (3×10 mL). The combined organic layers were washed with brine (3×10 mL), dried over anhydrous Na₂SO₄, filtered and the filtrate was concentrated under reduced pressure to give (2³S,6³ S,4S)-4-amino-1²-(2-((S)-1-methoxyethyl)pyridin-3-yl)-10,10-dimethyl-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-piperidinacycloundecaphane-5,7-dione (200 mg) as an oil. LCMS (ESI): m/z [M+H]⁺ calc'd for C₃₆H₄₇F₃N₆O₄ 684.4; found 985.4.
Step 2. To a mixture of (2³S,6³S,4S)-4-amino-1²-(2-((S)-1-methoxyethyl)pyridin-3-yl)-10,10-dimethyl-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)- piperidinacycloundecaphane-5,7-dione (200 mg, 0.29 mmol) in DMF (2 mL) at 0° C. under an atmosphere of N₂ was added DIPEA (378 mg, 2.9 mmol), (2S)-2-[(4aR,7aS)-4-(tert-butoxycarbonyl)-hexahydropyrrolo[3,4-b][1,4] oxazine-6-carbonyl (methyl)amino]-3-methylbutanoic acid (169 mg, 0.44 mmol) and HATU (133 mg, 0.35 mmol). The mixture was warmed to rt and stirred for 2 h, then H₂O added, and the mixture was extracted with EtOAc (3×10 mL). The combined organic layers were washed with brine (3×10 mL), dried over anhydrous Na₂SO₄, and filtered. The filtrate was concentrated under reduced pressure and the crude residue was purified by preparative-TLC to give tert-butyl (4aR,7aS)-6-(((2S)-1-(((2³S,6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-piperidinacycloundecaphane-4-yl)amino)-3-methyl-1-oxobutan-2-yl)(methyl)carbamoyl)hexahydropyrrolo[3,4-b][1,4]oxazine-4(4aH)-carboxylate (230 mg, 67% yield) as a solid. LCMS (ESI): m/z [M+H]⁺ calc'd for C₅₄H₇₆F₃N₉O₉ 1051.6; found 1052.6.
Step 3. To a mixture of tert-butyl (4aR,7aS)-6-(((2S)-1-(((2³S,6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-piperidinacycloundecaphane-4-yl)amino)-3-methyl-1-oxobutan-2-yl)(methyl) carbamoyl)hexahydropyrrolo[3,4-b][1,4]oxazine-4(4aH)-carboxylate (230 mg, 0.22 mmol) in DCM (3 mL) at 0° C. under an atmosphere of N₂ was added TFA. The mixture was warmed to rt and stirred for 2 h, then H₂O added, and the mixture was extracted with DCM (3×10 mL). The combined organic layers were washed with brine (3×10 mL), dried over anhydrous Na₂SO₄, filtered and the filtrate was concentrated under reduced pressure to give (4aR,7aS)—N-((2S)-1-(((2³S,6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-piperidinacycloundecaphane-4-yl)amino)-3-methyl-1-oxobutan-2-yl)-N-methylhexahydropyrrolo[3,4-b][1,4]oxazine-6(2H)-carboxamide (220 mg) as a solid. LCMS (ESI): m/z [M+H]⁺ calc'd for C₄₉H₆₈F₃N₉O₇ 951.5; found 952.5.
Step 4. To a mixture of (4aR,7aS)—N-((2S)-1-(((2³S,6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)- pyridazina-2(3,1)-piperidinacycloundecaphane-4-yl)amino)-3-methyl-1-oxobutan-2-yl)-N-methylhexahydropyrrolo[3,4-b][1,4]oxazine-6(2H)-carboxamide (220 mg, 0.23 mmol) in ACN (3 mL) at 0° C. under an atmosphere of N₂ was added DIPEA (299 mg, 2.3 mmol), acrylic acid (25 mg, 0.35 mmol) and CIP (77 mg, 0.28 mmol). The mixture was warmed to rt and stirred for 2 h, then H₂O added, and the mixture was extracted with EtOAc (3×10 mL). The combined organic layers were washed with brine (3×10 mL), dried over anhydrous Na₂SO₄, and filtered. The filtrate was concentrated under reduced pressure and the crude residue was purified by preparative-HPLC to give (4aR,7aS)-4-acryloyl-N-((2S)-1-(((2³S,6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-piperidinacycloundecaphane-4-yl)amino)-3-methyl-1-oxobutan-2-yl)-N-methylhexahydropyrrolo[3,4-b][1,4]oxazine-6(2H)-carboxamide (20 mg, 8% yield) as a solid. LCMS (ESI): m/z [M+H]⁺ calc'd for C₅₂H₇₀F₃N₉O₈ 1005.5; found 1006.9; ¹H NMR (300 MHZ, DMSO-d₆) δ 8.75 (dd, J=4.7, 1.8 Hz, 1H), 7.77 (d, J=7.9 Hz, 1H), 7.67 (t, J=9.2 Hz, 2H), 7.58-7.48 (m, 2H), 7.17 (d, J=8.6 Hz, 1H), 6.86 (dd, J=17.2, 10.6 Hz, 1H), 6.20 (d, J=16.5 Hz, 1H), 5.80-5.59 (m, 2H), 5.48 (s, 1H), 5.11 (d, J=11.7 Hz, 1H), 4.73 (d, J=15.3 Hz, 2H), 4.35 (d, J=12.8 Hz, 1H), 4.21-4.04 (m, 2H), 3.99-3.71 (m, 6H), 3.67-3.49 (m, 3H), 3.30-3.05 (m, 7H), 3.04-2.91 (m, 3H), 2.77-2.60 (m, 9H), 2.09 (d, J=42.2 Hz, 5H), 1.81 (d, J=28.6 Hz, 2H), 1.64-1.56 (m, 5H), 1.40 (d, J=6.1 Hz, 3H), 0.95 (s, 3H), 0.82 (t, J=6.4 Hz, 6H), 0.21 (s, 3H).

### Example 5. Synthesis of 4-acryloyl-N-((2S)-1-(((6³S,4S,Z)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(4,2)-oxazola-1(5,3)-indola-6(1,3)-pyridazinacycloundecaphane-4-yl)amino)-3-methyl-1-oxobutan-2-yl)-N-methyl-1-oxa-4,9-diazaspiro[5.5]undecane-9-carboxamide (A79)

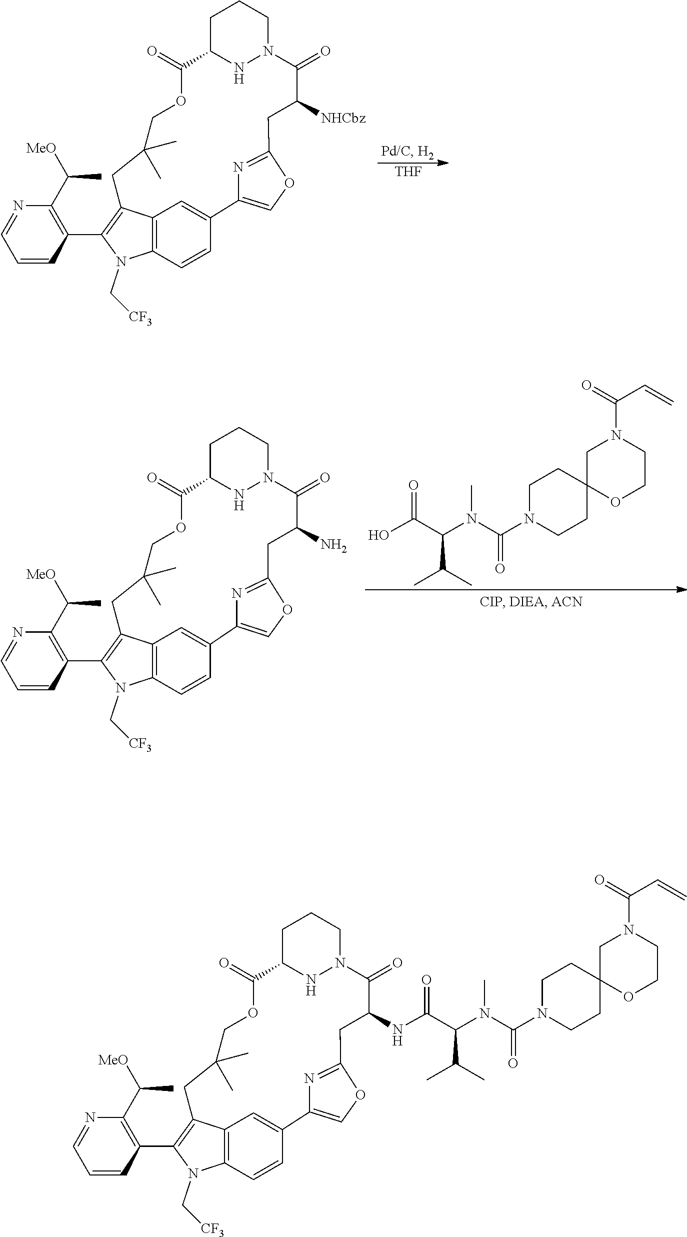

Step 1. A mixture of benzyl ((6³S,4S,Z)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(4,2)-oxazola-1(5,3)-indola-6(1,3)- pyridazinacycloundecaphane-4-yl)carbamate (Intermediate 16) (150 mg, 0.19 mmol) and 10% Pd/C (0.1 g) in THF (2 mL) was stirred at 35° C. under an atmosphere of H₂ (balloon) for 1 h. The mixture was filtered through a pad of Celite and the filtrate was concentrated under reduced pressure to give (6³S,4S,Z)-4-amino-1²-(2-((S)-1-methoxyethyl)pyridin-3-yl)-10,10-dimethyl-1¹-(2,2,2-trifluoroethyl)-6¹,6²,6³,6⁴,6⁵,6⁶-hexahydro-1¹H-8-oxa-2(4,2)-oxazola-1(5,3)-indola-6(1,3)-pyridazinacycloundecaphane-5,7-dione (112 mg, 90% yield) as a solid. LCMS (ESI): m/z [M+H]⁺ calc'd for C₃₄H₃₉F₃N₆O₅ 668.3; found 669.3.
Step 2. To a mixture of (6³S,4S,Z)-4-amino-1²-(2-((S)-1-methoxyethyl)pyridin-3-yl)-10,10-dimethyl-1¹-(2,2,2-trifluoroethyl)-6¹,6²,6³,6⁴,6⁵,6⁶-hexahydro-1¹H-8-oxa-2(4,2)-oxazola-1(5,3)-indola-6(1,3)-pyridazinacycloundecaphane-5,7-dione (91 mg, 0.14 mmol) in ACN (1 mL) at 0° C. was added DIPEA (352 mg, 2.7 mmol) and (2S)-3-methyl-2-[methyl (4-(prop-2-enoyl)-1-oxa-4,9-diazaspiro[5.5]undecane-9-carbonyl)amino]butanoic acid (75 mg, 0.20 mmol) and 2-chloro-1,3-dimethyl-4,5-dihydro-1H-imidazol-3-ium; hexafluorophosphate(V) (46 mg, 0.16 mmol). The mixture was stirred at 0° C. for 1 h, then concentrated under reduced pressure and the residue was purified by preparative-HPLC to give 4-acryloyl-N-((2S)-1-(((6³S,4S,Z)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(4,2)-oxazola-1(5,3)-indola-6(1,3)-pyridazinacycloundecaphane-4-yl)amino)-3-methyl-1-oxobutan-2-yl)-N-methyl-1-oxa-4,9-diazaspiro[5.5]undecane-9-carboxamide (29.6 mg, 21% yield) as a solid. LCMS (ESI): m/z [M+H]⁺ calc'd for C₅₂H₆₆F₃N₉O₉ 1017.5; found 1018.7; ¹H NMR (400 MHZ, DMSO-d₆) δ8.77 (dd, J=4.7, 1.8 Hz, 1H), 8.45-8.21 (m, 3H), 7.94-7.70 (m, 2H), 7.63 (d, J=7.6 Hz, 1H), 7.54 (m, 1H), 6.84 (t, J=13.8 Hz, 1H), 6.16 (d, J=16.5 Hz, 1H), 5.70 (d, J=10.5 Hz, 1H), 5.62-5.50 (m, 2H), 5.08 (d, J=11.9 Hz, 1H), 4.94-4.75 (m, 1H), 4.35 (td, J=12.1, 3.2 Hz, 1H), 4.34-4.15 (m, 2H), 3.94-3.80 (m, 1H), 3.65 (d, J=5.0 Hz, 2H), 3.57-3.48 (m, 6H), 3.28 (s, 4H), 3.19-2.93 (m, 4H), 2.93-2.62 (m, 5H), 2.40 (d, J=14.4 Hz, 1H), 2.20-2.04 (m, 2H), 1.86-1.57 (m, 5H), 1.58-1.40 (m, 2H), 1.37 (d, J=6.1 Hz, 3H), 0.98-0.77 (m, 9H), 0.28 (s, 3H).

### Example 6. Synthesis of 4-acryloyl-N-((2S)-1-(((6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-azetidinacycloundecaphane-4-yl)amino)-3-methyl-1-oxobutan-2-yl)-N-methyl-1-oxa-4,9-diazaspiro[5.5]undecane-9-carboxamide (A94)

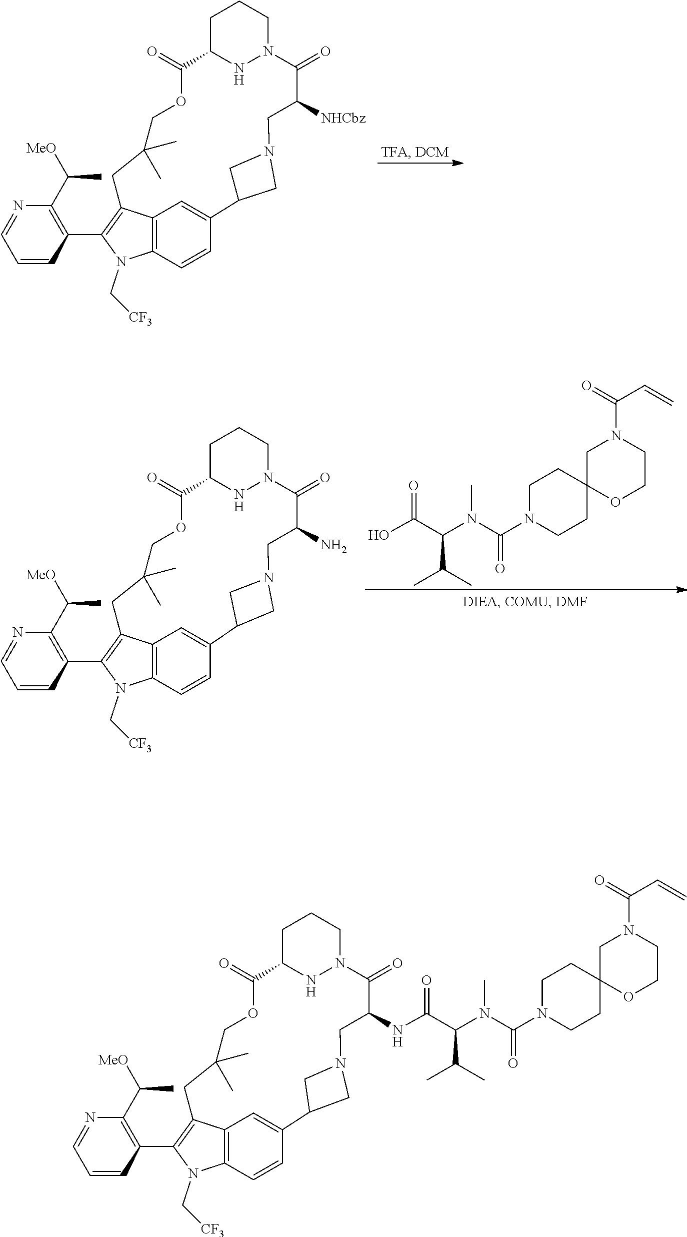

Step 1. To a mixture of tert-butyl ((6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)- azetidinacycloundecaphane-4-yl)carbamate (Intermediate 18) (100 mg, 0.13 mmol) in DCM (2 mL) at 0° C. was added TFA (301 mg, 2.64 mmol). The mixture was stirred at 0° C. for 4 h, then concentrated under reduced pressure to give (6³S,4S)-4-amino-1²-(2-((S)-1-methoxyethyl)pyridin-3-yl)-10,10-dimethyl-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-azetidinacycloundecaphane-5,7-dione (80 mg, 92% yield) as a solid. LCMS (ESI): m/z [M+H]⁺ calc'd for C₃₄H₄₃F₃N₆O₄ 656.3; found 657.5.
Step 2. To a mixture of (6³S,4S)-4-amino-1²-(2-((S)-1-methoxyethyl)pyridin-3-yl)-10,10-dimethyl-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-azetidinacycloundecaphane-5,7-dione (90 mg, 0.14 mmol) in DMF (2 mL) at 0° C. was added DIPEA (106 mg, 0.82 mmol), (2S)-3-methyl-2-[methyl (4-(prop-2-enoyl)-1-oxa-4,9-diazaspiro[5.5]undecane-9-carbonyl)amino]butanoic acid (76 mg, 0.21 mmol) and COMU (88 mg, 0.21 mmol). The mixture was stirred at 0° C. for 1 h, then H₂O was added, and the mixture was extracted with EtOAc (3×20 mL). The combined organic layers were washed with brine (20 mL), dried over anhydrous Na₂SO₄, and filtered. The filtrate was concentrated under reduced pressure and the residue was purified by preparative-HPLC to give 4-acryloyl-N-((2S)-1-(((6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-azetidinacycloundecaphane-4-yl)amino)-3-methyl-1-oxobutan-2-yl)-N-methyl-1-oxa-4,9-diazaspiro[5.5]undecane-9-carboxamide (37 mg, 27% yield) as a solid. LCMS (ESI): m/z [M+H]⁺ calc'd for C₅₂H₇₀F₃N₉O₈ 1005.5; found 1006.8; ¹H NMR (400 MHz, DMSO-d₆) δ 8.73 (dd, J=4.7, 1.8 Hz, 1H), 7.80 (s, 1H), 7.71-7.69 (m, 2H), 7.58-7.46 (m, 2H), 7.10 (d, J=8.4 Hz, 1H), 6.86-6.71 (m, 1H), 6.11 (dd, J=16.3, 9.7 Hz, 1H), 5.65 (t, J=8.3 Hz, 1H), 5.46 (dq, J=17.2, 8.8 Hz, 1H), 5.29-5.15 (m, 2H), 4.87-4.74 (m, 1H), 4.23 (d, J=12.3 Hz, 1H), 4.11 (q, J=6.0 Hz, 1H), 4.07-3.97 (m, 1H), 3.86-3.71 (m, 2H), 3.61-3.47 (m, 12H), 3.23 (m, 5H), 3.07-2.87 (m, 5H), 2.78 (s, 3H), 2.76-2.66 (m, 1H), 2.32 (d, J−14.4 Hz, 1H), 2.18-2.05 (m, 1H), 2.04-1.94 (m, 1H), 1.78 (d, J=10.0 Hz, 1H), 1.71 (d, J=13.3 Hz, 1H), 1.58 (dd, J=16.6, 6.9 Hz, 4H), 1.48-1.38 (m, 1H), 1.32 (d, J=6.0 Hz, 3H), 0.88-0.75 (m, 9H), 0.24 (s, 3H).

### Example 7. Synthesis of (2R)-3-acryloyl-N-((2S)-1-(((6³S,4S)-1¹-ethyl-1²-(2-((S)-1-methoxyethyl)pyridin-3-yl)-10,10-dimethyl-5,7-dioxo-2¹,2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-4-yl)amino)-3-methyl-1-oxobutan-2-yl)-N,2-dimethyl-1-oxa-3,8-diazaspiro[4.5]decane-8-carboxamide (A63)

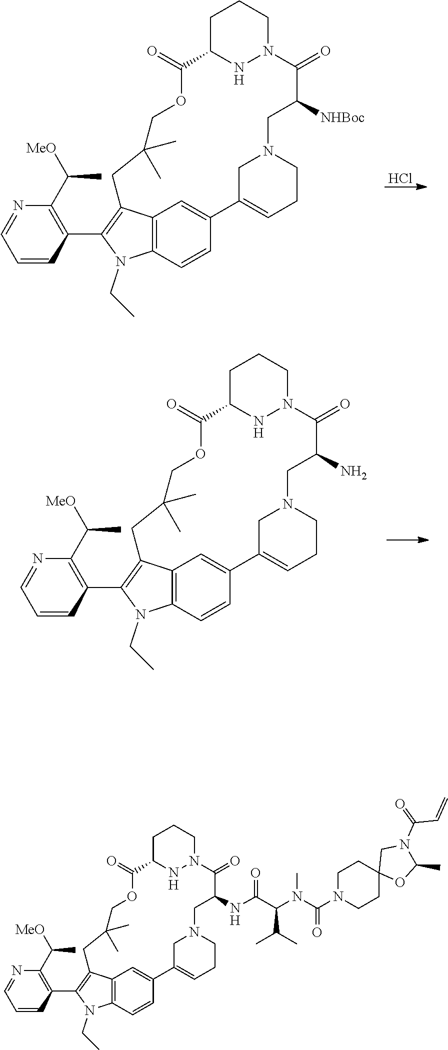

Step 1. To a mixture of methyl N—((R)-3-acryloyl-2-methyl-1-oxa-3,8-diazaspiro[4.5]decane-8-carbonyl)-N-methyl-L-valinate (430 mg, 1.127 mmol, 1.00 equiv) in THF (4 mL) and H₂O (4 mL) was added NaOH (225 mg, 5.6 mmol). The mixture was stirred at rt for 16 hours at rt, then acidified to PH ˜5 with 1M HCl and the mixture was extracted with EtOAc (4×10 mL). The combined organic layers were washed with brine (3 mL), dried over anhydrous Na₂SO₄, filtered and the filtrate was concentrated under reduced pressure to give N—((R)-3-acryloyl-2-methyl-1-oxa-3,8-diazaspiro[4.5]decane-8-carbonyl)-N-methyl-L-valine (300 mg) as a solid. LCMS (ESI): m/z [M+H]⁺ calc'd for C₁₈H₂₉N₃O₅ 367.2; found 368.3.
Step 2. To a mixture of tert-butyl ((6³S,4S)-1¹-ethyl-1²-(2-((S)-1-methoxyethyl)pyridin-3-yl)-10,10-dimethyl-5,7-dioxo-2¹,2²,2³,2⁶,6¹,6²,6³,6⁴,6⁵,6⁶-decahydro-1¹H-8-oxa-1(5,3)-indola-6(1,3)-pyridazina-2(5,1)- pyridinacycloundecaphane-4-yl)carbamate (1.0 g, 1.4 mmol) in DCM (10 mL) at 0° C. was added HCl in 1,4-dioxane (5 mL). The mixture was stirred at 0° C. for 2 h, then concentrated under reduced pressure to give (6³S,4S)-4-amino-1¹-ethyl-1²-(2-((S)-1-methoxyethyl)pyridin-3-yl)-10,10-dimethyl-2¹,2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-5,7-dione HCl (1.0 g) as a solid. LCMS (ESI): m/z [M+H]⁺ calc'd for C₃₆H₄₈N₆O₄ 628.4; found 629.6.
Step 3. To a mixture of (6³S,4S)-4-amino-1¹-ethyl-1²-(2-((S)-1-methoxyethyl)pyridin-3-yl)-10,10-dimethyl-2¹,2²,2³,2⁶,6¹,6²,6³,6⁴,6⁵,6⁶-decahydro-1¹H-8-oxa-1(5,3)-indola-6(1,3)-pyridazina-2(5,1)- pyridinacycloundecaphane-5,7-dione HCl (460 mg, 0.73 mmol) and N—((R)-3-acryloyl-2-methyl-1-oxa-3,8-diazaspiro[4.5]decane-8-carbonyl)-N-methyl-L-valine (269 mg, 0.73 mmol) in DMF (5 mL) at 0° C. was added DIPEA (2.84 g, 22.0 mmol) and COMU (282 mg, 0.66 mmol). The mixture was stirred at 0° C. for 1 h, then H₂O was added, and the mixture was extracted with EtOAc (5×10 mL). The combined organic layers were washed with brine (3×6 mL), dried over anhydrous Na₂SO₄, and filtered. The filtrate was concentrated under reduced pressure and the residue was purified by preparative-HPLC to give (2R)-3-acryloyl-N-((2S)-1-(((6³S,4S)-1¹-ethyl-1²-(2-((S)-1-methoxyethyl)pyridin-3-yl)-10,10-dimethyl-5,7-dioxo-2¹,2²,2³,2⁶,6¹,6²,6³,6⁴,6⁵,6⁶-decahydro-1¹H-8-oxa-1(5,3)-indola-6(1,3)-pyridazina-2(5,1)- pyridinacycloundecaphane-4-yl)amino)-3-methyl-1-oxobutan-2-yl)-N,2-dimethyl-1-oxa-3,8-diazaspiro[4.5]decane-8-carboxamide (50 mg, 7% yield) as a solid. LCMS (ESI): m/z [M+H]⁺ calc'd for C₅₄H₇₅N₉O₈ 977.6; found 978.6; ¹H NMR (400 MHZ, DMSO-d₆) δ 8.83-8.67 (m, 1H), 7.89 (dd, J=18.7, 8.2 Hz, 2H), 7.62-7.33 (m, 4H), 6.57 (dd, J=16.7, 10.3 Hz, 1H), 6.38-6.11 (m, 2H), 5.75 (d, J=9.8 Hz, 2H), 5.61 (d, J=11.8 Hz, 1H), 5.35 (d, J=5.5 Hz, 1H), 4.30 (d, J=12.7 Hz, 1H), 4.16 (q, J=6.2 Hz, 1H), 4.04 (s, 2H), 3.92-3.68 (m, 4H), 3.63 (s, 2H), 3.18 (d, J=61.5 Hz, 6H), 2.95 (d, J=33.8 Hz, 5H), 2.78 (t, J=11.8 Hz, 1H), 2.64 (d, J=24.7 Hz, 7H), 2.42-1.83 (m, 7H), 1.89-1.45 (m, 7H), 1.40 (dd, J=11.9, 5.7 Hz, 6H), 1.10 (t, J=7.0 Hz, 3H), 0.94-0.64 (m, 9H), 0.52 (s, 3H).

### Example 8. Synthesis of 3-acryloyl-N-((2S)-1-(((23 S,63S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-pyrrolidinacycloundecaphane-4-yl)amino)-3-methyl-1-oxobutan-2-yl)-N-methyl-1-oxa-3,8-diazaspiro[4.5]decane-8-carboxamide (A66)

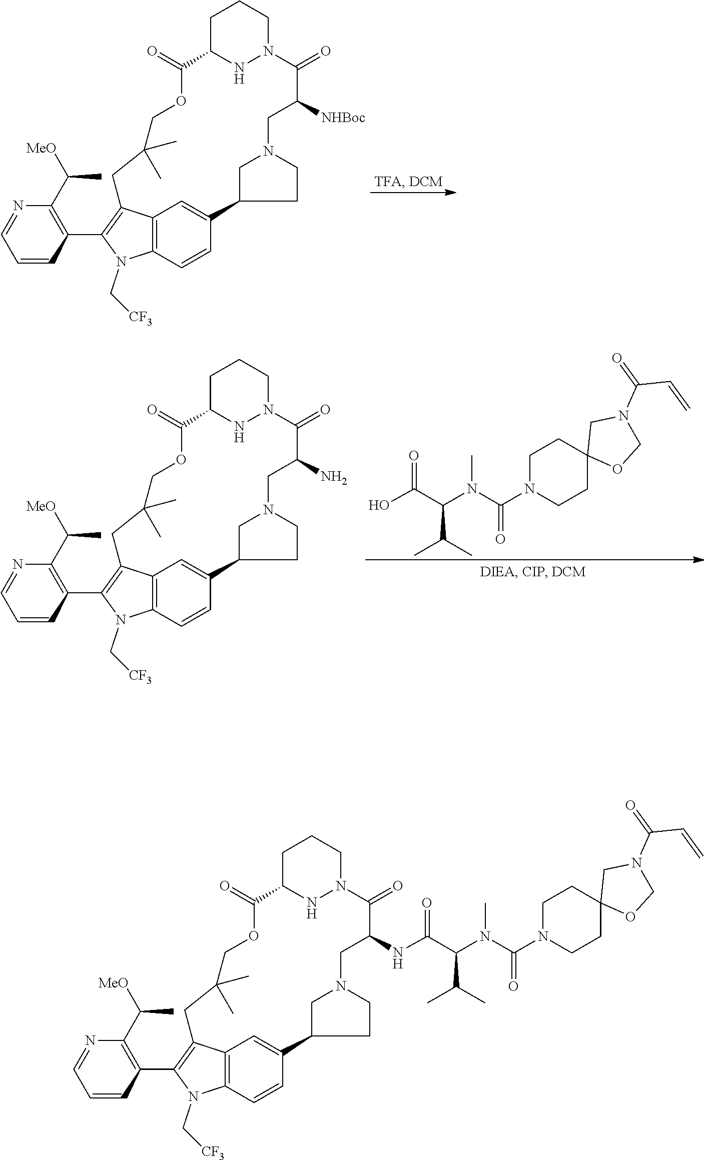

Step 1. To a mixture of tert-butyl ((6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-pyrrolidinacycloundecaphane-4-yl)carbamate (Intermediate 17) (410 mg, 0.53 mmol) in DCM (5 mL) at 0° C. was added TFA (1.7 mL, 22.9 mmol). The mixture was warmed to rt and stirred for 1 h, then basified to PH ˜6 with saturated NaHCO₃ and the mixture was extracted with EtOAc (6×3 mL). The combined organic layers were washed with brine (5×3 mL), dried over anhydrous Na₂SO₄, filtered and the filtrate was concentrated under reduced pressure to give (2³S,6³S,4S)-4-amino-1²-(2-((S)-1-methoxyethyl) pyridin-3-yl)-10,10-dimethyl-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-pyrrolidinacycloundecaphane-5,7-dione (390 mg) as a solid. LCMS (ESI): m/z [M+H]⁺ calc'd for C₃₅H₄₅F₃N₆O₄ 670.4; found 671.7.
Step 2. To a mixture of (2³S,6³S,4S)-4-amino-1²-(2-((S)-1-methoxyethyl)pyridin-3-yl)-10,10-dimethyl-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)- pyrrolidinacycloundecaphane-5,7-dione (270 mg, 0.4 mmol) and DIPEA (2.1 g, 16.1 mmol) in DCM (3 mL) at 0° C. under an atmosphere of N₂ was added (2S)-3-methyl-2-[methyl (3-(prop-2-enoyl)-1-oxa-3,8-diazaspiro[4.5]decane-8-carbonyl)amino]butanoic acid (142 mg, 0.4 mmol) and CIP (227 mg, 0.81 mmol). The mixture was stirred at rt for 30 min, then H₂O was added, and the mixture was extracted with EtOAc (4×30 mL). The combined organic layers were washed with brine (5×30 mL), dried over anhydrous Na₂SO₄, and filtered. The filtrate was concentrated under reduced pressure and the residue was purified by preparative-HPLC to give 3-acryloyl-N-((2S)-1-(((2³S,6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)- pyrrolidinacycloundecaphane-4-yl)amino)-3-methyl-1-oxobutan-2-yl)-N-methyl-1-oxa-3,8-diazaspiro[4.5]decane-8-carboxamide (45 mg, 10% yield) as a solid. LCMS (ESI): m/z [M+H]⁺ calc'd for C₅₂H₇₀F₃N₉O₈ 1005.5; found 1006.9; ¹H NMR (400 MHZ, DMSO-d₆) δ 8.76 (dd, J=4.7, 1.8 Hz, 1H), 7.81 (d, J=8.8 Hz, 1H), 7.74 (d, J=7.7 Hz, 1H), 7.60 (d, J=8.4 Hz, 1H), 7.58-7.50 (m, 2H), 7.13 (d, J=8.2 Hz, 1H), 6.54 (dd, J=16.8, 10.3 Hz, 1H), 6.24-6.14 (m, 1H), 5.74 (td, J=10.2, 2.3 Hz, 1H), 5.58 (q, J=6.9 Hz, 1H), 5.46 (dt, J=17.2, 8.7 Hz, 1H), 5.13 (d, J=13.2 Hz, 2H), 5.01 (s, 1H), 4.81 (dt, J=18.2, 9.0 Hz, 1H), 4.31 (d, J=12.4 Hz, 1H), 4.20 (q, J=6.0 Hz, 1H), 3.87 (s, 1H), 3.80 (d, J=11.0 Hz, 1H), 3.67 (s, 2H), 3.60-3.55 (m, 1H), 3.45 (s, 1H), 3.12 (dt, J=17.2, 9.6 Hz, 3H), 2.76 (d, J=13.0 Hz, 5H), 2.61 (q, J=7.8, 6.9 Hz, 2H), 2.42 (d, J=14.4 Hz, 1H), 2.29-1.87 (m, 4H), 1.80 (t, J=12.5 Hz, 3H), 1.65 (dt, J=22.2, 8.9 Hz, 3H), 1.58-1.48 (m, 2H), 1.38 (d, J=6.0 Hz, 3H), 0.98-0.83 (m, 6H), 0.81 (d, J=6.6 Hz, 3H), 0.26 (s, 3H).

### Example 9. Synthesis of 4-acryloyl-N-(2S)-1-(((6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methyl-1-oxa-4,9-diazaspiro[5.5]undecane-9-carboxamide (A60)

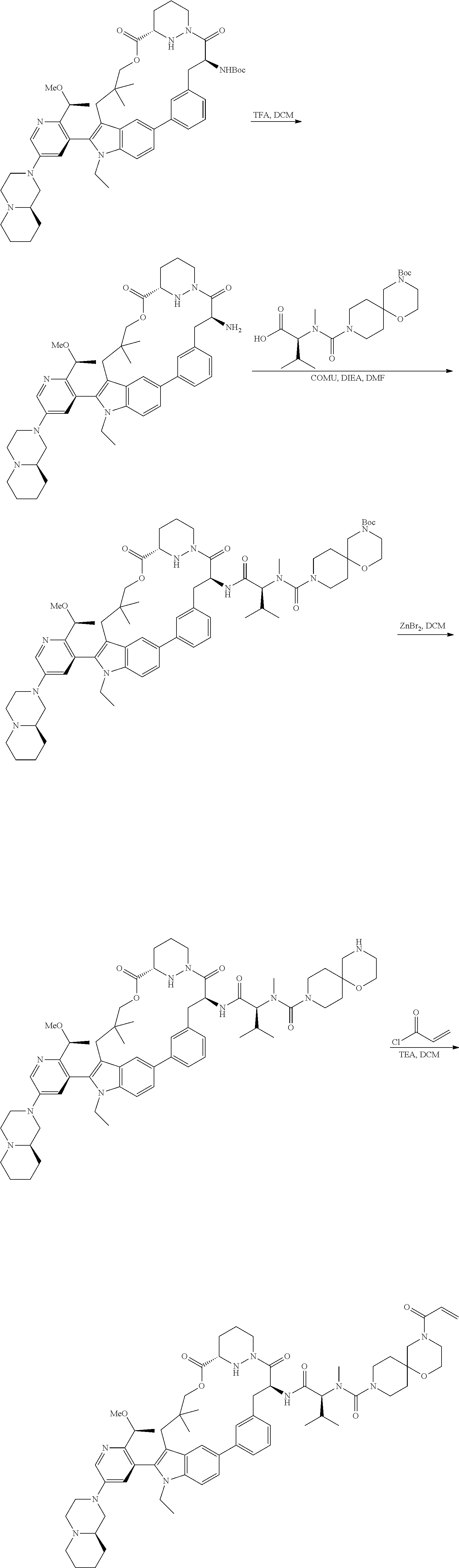

Step 1. To a mixture of tert-butyl ((6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (Intermediate 19) (230 mg, 0.27 mmol) in DCM (2 mL) at 0° C. was added TFA (1 mL) dropwise. The mixture was stirred at 0° C. for 1 h, then basified to PH ˜8 with saturated NaHCO₃ at 0° C. and the mixture extracted with EtOAc (3×30 mL). The combined organic layers were washed with brine (3×30 mL), dried over anhydrous Na₂SO₄, filtered and the filtrate was concentrated under reduced pressure to give (6³S,4S)-4-amino-1¹-ethyl-1²-(2-((S)-1-methoxyethyl)-5-((R)-octahydro-2H-pyrido[1,2-a]pyrazin-2-yl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (300 mg) as a solid, that was used in the next step without further purification. LCMS (ESI): m/z [M+H]⁺ calc'd for C₄₅H₅₉N₇O₄ 761.5; found 762.8.
Step 2. To a mixture of (6³S,4S)-4-amino-1¹-ethyl-1²-(2-((S)-1-methoxyethyl)-5-((R)-octahydro-2H-pyrido[1,2-a]pyrazin-2-yl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (300 mg, 0.39 mmol) and (2S)-2-[4-(tert-butoxycarbonyl)-1-oxa-4,9-diazaspiro[5.5]undecane-9-carbonyl (methyl)amino]-3-methylbutanoic acid (211 mg, 0.51 mmol) in DMF (3 mL) at 0° C. under an atmosphere of Ar was added DIPEA (1.53 g, 11.8 mmol) and COMU (168 mg, 0.39 mmol) in DMF (0.1 mL) dropwise. The mixture was stirred at 0° C. for 1 h, then ice/H₂O (3 mL) was added, and the mixture was extracted with EtOAc (3×30 mL). The combined organic layers were washed with brine (3×30 mL), dried over anhydrous Na₂SO₄, and filtered. The filtrate was concentrated under reduced pressure and the residue was purified to give tert-butyl 9-(((2S)-1-(((6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)-1-oxa-4,9-diazaspiro[5.5]undecane-4-carboxylate (200 mg, 59% yield) as a solid. LCMS (ESI): m/z [M+H]⁺ calc'd for C₆₅H₉₂N₁₀O₉ 1156.7; found 1158.2.
Step 3. A mixture of tert-butyl 9-(((2S)-1-(((6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)-1-oxa-4,9-diazaspiro[5.5]undecane-4-carboxylate (200 mg, 0.17 mmol) and ZnBr₂ (195 mg, 0.87 mmol) in DCM (4 mL) was heated to 35° C. and stirred overnight. Ice/H₂O (5 mL) was added, and the mixture was basified to PH ˜8 with saturated NaHCO₃ at 0° C., then extracted with EtOAc (3×30 mL). The combined organic layers were washed with brine (3×30 mL), dried over anhydrous Na₂SO₄, filtered and the filtrate was concentrated under reduced pressure to give N-((2S)-1-(((6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methyl-1-oxa-4,9-diazaspiro[5.5]undecane-9-carboxamide (200 mg) as a solid. LCMS (ESI): m/z [M+H]⁺ calc'd for C₆₀H₈₄N₁₀O₇ 1056.7; found 1058.1.
Step 4. To a mixture of N-((2S)-1-(((6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methyl-1-oxa-4,9-diazaspiro[5.5]undecane-9-carboxamide (200 mg, 0.19 mmol) and TEA (57 mg, 0.57 mmol) in DCM (2 mL) at 0° C. under an atmosphere of Ar was added acryloyl chloride (12 mg, 0.13 mmol) dropwise. The mixture was stirred at 0° C. for additional 1 h, then concentrated under reduced pressure and the crude residue was purified by preparative-HPLC to give 4-acryloyl-N-((2S)-1-(((6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methyl-1-oxa-4,9-diazaspiro[5.5]undecane-9-carboxamide (40 mg, 19% yield) as a solid. LCMS (ESI): m/z [M+H]⁺ calc'd for C₆₃H₈₆N₁₀O₈ 1110.7; found 1112.1; ¹H NMR (400 MHz, DMSO-d₆) δ 8.46 (d, J=2.8 Hz, 1H), 8.17-8.05 (m, 1H), 7.98 (s, 1H), 7.86 (s, 1H), 7.74-7.54 (m, 3H), 7.27-7.19 (m, 2H), 7.01-6.81 (m, 2H), 6.28-6.11 (m, 1H), 5.73 (d, J=10.3 Hz, 1H), 5.43 (d, J=9.4 Hz, 2H), 4.40-4.17 (m, 2H), 4.10 (dq, J=21.9, 7.1, 6.5 Hz, 2H), 3.95 (t, J=12.0 Hz, 1H), 3.77 (dt, J=25.3, 13.0 Hz, 3H), 3.69-3.64 (m, 3H), 3.64-3.55 (m, 3H), 3.54-3.48 (m, 2H), 3.15 (d, J=11.7 Hz, 2H), 3.07 (s, 3H), 2.97-2.89 (m, 1H), 2.79 (m, 4H), 2.66 (s, 1H), 2.56 (s, 3H), 2.42 (d, J=11.1 Hz, 1H), 2.23 (td, J=11.6, 3.2 Hz, 1H), 2.07-1.89 (m, 4H), 1.82 (d, J=12.2 Hz, 1H), 1.77-1.63 (m, 4H), 1.59 (d, J=12.6 Hz, 3H), 1.47 (d, J=13.1 Hz, 2H), 1.36 (d, J=6.1 Hz, 3H), 1.19 (m, 3H), 1.00 (t, J=7.1 Hz, 3H), 0.90-0.70 (m, 9H), 0.57 (s, 3H).

### Example 10. Synthesis of (3S)-1-acryloyl-N-((2S)-1-(((6³S)-1¹-ethyl-1²-(2-((S)-1-methoxyethyl)pyridin-3-yl)-10,10-dimethyl-2¹,2¹-dioxido-5,7-dioxo-6¹,6²,6³,6⁴,6⁵,6⁶-hexahydro-1¹H-8-oxa-2(4,2)-thiomorpholina-1(5,3)-indola-6(1,3)-pyridazinacycloundecaphane-4-yl)amino)-3-methyl-1-oxobutan-2-yl)-N-methylpyrrolidine-3-carboxamide (C195)

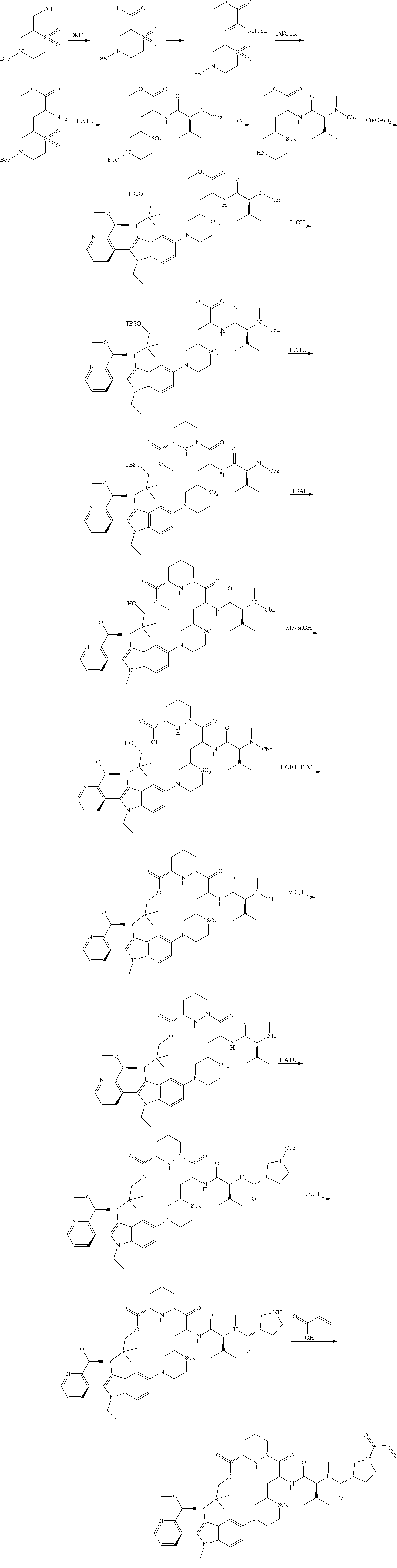

Step 1. To a mixture of tert-butyl 2-(hydroxymethyl)thiomorpholine-4-carboxylate 1,1-dioxide (17.8 g, 60 mmol) in DCM (200 mL) was added Dess-Martin periodinane (56.6 g, 130 mmol). The mixture was stirred at rt for 2 h, then filtered and the filtrate was concentrated under reduced pressure to give tert-butyl 2-formylthiomorpholine-4-carboxylate 1,1-dioxide (30 g) as a syrup, which was used in the next step without further purification. LCMS (ESI): m/z [M-^{t}Bu+H]⁺ calc'd for C₆H₉NO₅S 207.2; found 208.0; ¹H NMR (400 MHZ, CDCl₃) δ 9.88 (s, 1H), 4.17 (d, J=39.4, 33.7 Hz, 4H), 3.15 (d, J=34.2 Hz, 3H), 1.48 (s, 10H).
Step 2. To a mixture of tert-butyl 2-formylthiomorpholine-4-carboxylate 1,1-dioxide (58 g, 60 mmol) in ACN (400 mL) at 0° C. was added 1,1,3,3-tetramethylguanidine (30.5 g, 200 mmol) and methyl 2-{[(benzyloxy)carbonyl]amino}-2-(dimethoxyphosphoryl) acetate (43.8 g, 130 mmol). The mixture was warmed to rt and stirred for 2 h then concentrated under reduced pressure. The residue was diluted with EtOAc (200 mL) and washed with H₂O (150 mL×3), then dried and filtered. The filtrate was concentrated under reduced pressure and the residue was purified by silica gel column chromatography to give tert-butyl 2-(2-(((benzyloxy)carbonyl)amino)-3-methoxy-3-oxoprop-1-en-1-yl)thiomorpholine-4-carboxylate 1, 1-dioxide (8 g, 25% yield over 2 steps) as a syrup. LCMS (ESI): m/z [M+Na]⁺ calc'd for C₂₁H₂₈N₂NaO₈S 491.2; found 491.2.
Step 3. A mixture of tert-butyl 2-(2-(((benzyloxy)carbonyl)amino)-3-methoxy-3-oxoprop-1-en-1-yl)thiomorpholine-4-carboxylate (8 g, 17.0 mmol), 10% Pd/C (4 g) and NH₄Cl (9.1 g, 170 mmol) in MeOH (200 mL) was stirred at rt under an atmosphere of H₂ for 48 h. The mixture was filtered, and the filtrate was concentrated under reduced pressure to give tert-butyl 2-(2-amino-3-methoxy-3-oxopropyl)thiomorpholine-4-carboxylate 1,1-dioxide (6.3 g) as an oil, which was used in next step without further purification. LCMS (ESI): m/z [M+H]⁺ calc'd for C₁₃H₂₄N₂O₆S 336.1; found 337.1.
Step 4. To a mixture of tert-butyl 2-(2-amino-3-methoxy-3-oxopropyl)thiomorpholine-4-carboxylate 1,1-dioxide (6.3 g, 10 mmol) and (2S)-2-({3-[(formyloxy)methyl]phenyl}(methyl)amino)-3-methylbutanoic acid (5 g, 10 mmol) in dry DMF (20 mL) at 0° C. was added DIPEA (49.2 g, 30 mmol) and HATU (7.2 g, 10 mmol). The mixture was stirred at 0° C. for 1 h, then diluted with EtOAc (100 mL) and washed with H₂O (50 mL×3). The combined organic layers were concentrated under reduced pressure and the residue was purified by silica gel column chromatography to give tert-butyl 2-(2-((S)-2-(((benzyloxy)carbonyl)(methyl)amino)-3-methylbutanamido)-3-methoxy-3-oxopropyl)thiomorpholine-4-carboxylate 1,1-dioxide (5 g, 57% yield over 2 steps) as an oil. LCMS (ESI): m/z [M+H]⁺ calc'd for C₂₇H₄₁N₃O₉S 583.3; found 584.3.
Step 5. To a mixture of tert-butyl 2-(2-((S)-2-(((benzyloxy)carbonyl)(methyl)amino)-3-methylbutanamido)-3-methoxy-3-oxopropyl)thiomorpholine-4-carboxylate 1,1-dioxide (12 g, 20 mmol) in DCM (80 mL) at 0° C. was added TFA (20 mL). The mixture was warmed to rt and stirred for 1.5 h, then concentrated under reduced pressure. The residue was diluted with EtOAc (50 mL) and adjusted to PH ˜9 with saturated Na₂CO₃. The organic layer was concentrated under reduced pressure to give methyl 2-((S)-2-(((benzyloxy)carbonyl)(methyl)amino)-3-methylbutanamido)-3-(1,1-dioxidothiomorpholin-2-yl)propanoate (9.1 g, yield 94%) as a syrup, which was used in the next step without further purification. LCMS (ESI): m/z [M+H]⁺ calc'd for C₂₂H₃₃N₃O₇S 483.2; found 484.2.
Step 6. To a mixture of methyl 2-((S)-2-(((benzyloxy)carbonyl)(methyl)amino)-3-methylbutanamido)-3-(1,1-dioxidothiomorpholin-2-yl)propanoate (5.9 g, 12 mmol) in DCM (50 mL) at rt was added (3-{3-[(tert-butyldimethylsilyl)oxy]-2,2-dimethylpropyl}-1-ethyl-2-{2-[(1S)-1-methoxyethyl]pyridin-3-yl}indol-5-yl)boranediol (6.4 g, 12 mmol), Cu (OAc) 2 (2.2 g, 12 mmol) and pyridine (2.8 g, 36 mmol). The mixture was stirred at rt for 48 h, then the mixture was filtered, the filtrate was diluted with EtOAc (30 mL) and washed with H₂O (80 mL×3). The organic layer was concentrated under reduced pressure and the residue was purified by silica gel column chromatography to give methyl 2-((S)-2-(((benzyloxy)carbonyl)(methyl)amino)-3-methylbutanamido)-3-(4-((R)-3-(3-((tert-butyldimethylsilyl)oxy)-2,2-dimethylpropyl)-1-ethyl-2-(2-((S)-1-methoxyethyl)pyridin-3-yl)-1H-indol-5-yl)-1,1-dioxidothiomorpholin-2-yl)propanoate (7.59 g, 66% yield) as an oil. LCMS (ESI): m/z [M+H]⁺ calc'd for C₅₁H₇₅N₅O₉SSi 961.5; found 962.3.
Step 7. To a mixture of methyl 2-((S)-2-(((benzyloxy)carbonyl)(methyl)amino)-3-methylbutanamido)-3-(4-((R)-3-(3-((tert-butyldimethylsilyl)oxy)-2,2-dimethylpropyl)-1-ethyl-2-(2-((S)-1-methoxyethyl)pyridin-3-yl)-1H-indol-5-yl)-1,1-dioxidothiomorpholin-2-yl)propanoate (7.59 g, 7.9 mmol) in THF (40 mL) at 0° C. was added LiOH (0.38 g, 16 mmol) in H₂O (8 mL). The mixture was stirred at 0° C. for 1.5 h, then the pH adjusted to pH ˜7 with 3M HCl (5 mL), the mixture diluted with brine (15 mL) and extracted with EtOAc (50 mL×3). The combined organic layers were concentrated under reduced pressure to give 2-((S)-2-(((benzyloxy)carbonyl)(methyl)amino)-3-methylbutanamido)-3-(4-((R)-3-(3-((tert-butyldimethylsilyl)oxy)-2,2-dimethylpropyl)-1-ethyl-2-(2-((S)-1-methoxyethyl)pyridin-3-yl)-1H-indol-5-yl)-1,1-dioxidothiomorpholin-2-yl)propanoic acid (7.4 g, 98% yield) as a syrup. LCMS (ESI): m/z [M+H]⁺ calc'd for C₅₀H₇₃N₅O₉SSi 947.5; found 948.4.
Step 8. To a mixture of 2-((S)-2-(((benzyloxy)carbonyl)(methyl)amino)-3-methylbutanamido)-3-(4-((R)-3-(3-((tert-butyldimethylsilyl)oxy)-2,2-dimethylpropyl)-1-ethyl-2-(2-((S)-1-methoxyethyl)pyridin-3-yl)-1H-indol-5-yl)-1,1-dioxidothiomorpholin-2-yl)propanoic acid (7.4 g, 7.8 mmol) in DMF (50 mL) at 0° C. was added methyl (3S)-1,2-diazinane-3-carboxylate dihydrochloride (2.6 g, 12 mmol), DIPEA (20 g, 160 mmol) and HATU (4.6 g, 12 mmol). The mixture was stirred at 0° C. for 2 h, then diluted with EtOAc (300 mL) and washed with H₂O (100 mL×2). The combined organic layers were concentrated under reduced pressure and the residue was purified by silica gel column chromatography to give (3S)-methyl 1-(2-((S)-2-(((benzyloxy)carbonyl)(methyl)amino)-3-methylbutanamido)-3-(4-((R)-3-(3-((tert-butyldimethylsilyl)oxy)-2,2-dimethylpropyl)-1-ethyl-2-(2-((S)-1-methoxyethyl)pyridin-3-yl)-1H-indol-5-yl)-1,1-dioxidothiomorpholin-2-yl)propanoyl)hexahydropyridazine-3-carboxylate (8.08 g, 96% yield) as a syrup. LCMS (ESI): m/z [M+H]⁺ calc'd for C₅₆H₈₃N₇O₁₀SSi 1073.6; found 1074.5.
Step 9. To a mixture of 1M TBAF in THF (38 mL, 38 mmol) and AcOH (2.3 g, 38 mmol) was added (3S)-methyl 1-(2-((S)-2-(((benzyloxy)carbonyl)(methyl)amino)-3-methylbutanamido)-3-(4-((R)-3-(3-((tert-butyldimethylsilyl)oxy)-2,2-dimethylpropyl)-1-ethyl-2-(2-((S)-1-methoxyethyl)pyridin-3-yl)-1H-indol-5-yl)-1, 1-dioxidothiomorpholin-2-yl)propanoyl)hexahydropyridazine-3-carboxylate (8.08 g, 7.5 mmol). The mixture was heated to 55° C. and stirred for 16 h, then diluted with EtOAc (200 mL) and washed with H₂O (150 mL×2). The combined organic layers were concentrated under reduce pressure to give (3S)-methyl 1-(2-((S)-2-(((benzyloxy)carbonyl)(methyl)amino)-3-methylbutanamido)-3-(4-((R)-1-ethyl-3-(3-hydroxy-2,2-dimethylpropyl)-2-(2-((S)-1-methoxyethyl)pyridin-3-yl)-1H-indol-5-yl)-1,1-dioxidothiomorpholin-2-yl)propanoyl)hexahydropyridazine-3-carboxylate (7.2 g, 99% yield) as a syrup. LCMS (ESI): m/z [M+H]⁺ calc'd for C₅₀H₆₉N₇O₁₀S 959.5; found 960.3.
Step 10. To a mixture of (3S)-methyl 1-(2-((S)-2-(((benzyloxy)carbonyl)(methyl)amino)-3-methylbutanamido)-3-(4-((R)-1-ethyl-3-(3-hydroxy-2,2-dimethylpropyl)-2-(2-((S)-1-methoxyethyl)pyridin-3-yl)-1H-indol-5-yl)-1,1-dioxidothiomorpholin-2-yl)propanoyl)hexahydropyridazine-3-carboxylate (7.2 g, 7.5 mol) in DCE (30 mL) was added MesSnOH (6.7 g, 38 mmol). The mixture was heated to 65° C. and stirred for 16 h, then filtered and the filtrate was concentrated under reduced pressure to give (3S)-1-(2-((S)-2-(((benzyloxy)carbonyl)(methyl)amino)-3-methylbutanamido)-3-(4-((R)-1-ethyl-3-(3-hydroxy-2,2-dimethylpropyl)-2-(2-((S)-1-methoxyethyl)pyridin-3-yl)-1H-indol-5-yl)-1,1-dioxidothiomorpholin-2-yl)propanoyl)hexahydropyridazine-3-carboxylic acid (13 g) as an oil. LCMS (ESI): m/z [M+H]⁺ calc'd for C₄₉H₆₇N₇O₁₀S 945.5; found 946.4.
Step 11. To a mixture of (3S)-1-(2-((S)-2-(((benzyloxy)carbonyl)(methyl)amino)-3-methylbutanamido)-3-(4-((R)-1-ethyl-3-(3-hydroxy-2,2-dimethylpropyl)-2-(2-((S)-1-methoxyethyl)pyridin-3-yl)-1H-indol-5-yl)-1,1-dioxidothiomorpholin-2-yl)propanoyl)hexahydropyridazine-3-carboxylic acid (13 g, 7.4 mmol; ca. 55% purity) in DCM (400 mL) at 0° C. was added DIPEA (38 g, 300 mmol), HOBT (10 g, 74 mmol) and EDCl (42 g, 220 mmol). The mixture was warmed to rt and stirred for 48 h, then concentrated under reduced pressure, the residue diluted with EtOAc (200 mL) and washed with H₂O (100 mL×2). The organic layer was concentrated under reduced pressure and the residue was purified by silica gel chromatography to give benzyl ((2S)-1-(((6³S)-1¹-ethyl-1²-(2-((S)-1-methoxyethyl)pyridin-3-yl)-10,10-dimethyl-2¹,2¹-dioxido-5,7-dioxo-6¹,6²,6³,6⁴,6⁵,6⁶-hexahydro-1¹H-8-oxa-2(4,2)-thiomorpholina-1(5,3)-indola-6(1,3)-pyridazinacycloundecaphane-4-yl)amino)-3-methyl-1-oxobutan-2-yl)(methyl)carbamate [four isomers; a mixture of Isomer 1 and Isomer 2, 1.6 g; Isomer 3 (651 mg, 9.5% yield); Isomer 4 (332 mg, 4.8% yield)]. The mixture of Isomer 1 and Isomer 2 was purified further by preparative-HPLC to give Isomer 1 (470 mg, 6.8% yield) and Isomer 2 (797 mg, 12% yield).
Data for Isomer 1: LCMS (ESI): m/z [M+H]⁺ calc'd for C₄₉H₆₅NO₉S 927.5; found 928.4; ¹H NMR (400 MHZ, CD₃OD) δ 8.74 (dd, J=4.8, 1.6 Hz, 1H), 8.36-8.13 (m, 1H), 7.91 (dd, J=7.8, 1.7 Hz, 1H), 7.52 (dd, J=7.8, 4.8 Hz, 1H), 7.45-7.25 (m, 6H), 7.21-7.07 (m, J=8.8 Hz, 1H), 5.59-5.40 (m, 2H), 5.28-5.05 (m, 2H), 4.45 (d, 1H), 4.17 (d, J=11.0 Hz, 1H), 4.13-3.97 (m, 2H), 3.97-3.62 (m, 6H), 3.50-3.34 (m, 2H), 3.27-3.04 (m, 4H), 3.01-2.83 (m, 4H), 2.78 (s, 2H), 2.64-2.32 (m, 2H), 2.24-1.90 (m, 5H), 1.84-1.65 (m, 2H), 1.46 (dd, J=16.6, 6.6 Hz, 3H), 1.36-1.17 (m, 4H), 1.02 (s, 2H), 0.94-0.70 (m, 6H), 0.58 (s, 3H).
Data for Isomer 2: LCMS (ESI): m/z [M+H]⁺ calc'd for C₄₉H₆₅NO₉S 927.5; found 928.4; ¹H NMR (400 MHZ, CD₃OD) δ 8.71 (dd, J=4.8, 1.6 Hz, 1H), 8.18-8.01 (m, 1H), 7.83 (dd, J=7.7, 1.6 Hz, 1H), 7.52 (dd, J=7.7, 4.9 Hz, 1H), 7.45-7.23 (m, 6H), 7.20 (s, 1H), 7.06 (dd, J=8.9, 2.1 Hz, 1H), 5.66-5.50 (m, 1H), 5.29-5.05 (m, 2H), 4.36-4.18 (m, 3H), 4.17-4.09 (m, 2H), 4.05-3.86 (m, 5H), 3.75 (d, J=16.6 Hz, 1H), 3.54-3.36 (m, 2H), 3.27 (s, 1H), 3.21-3.06 (m, 4H), 3.03-2.91 (m, 1H), 2.88 (s, 3H), 2.81-2.63 (m, 2H), 2.47-2.35 (m, 1H), 2.34-2.09 (m, 3H), 2.00-1.93 (m, 1H), 1.86 (d, J=10.2 Hz, 1H), 1.79-1.63 (m, 2H), 1.43 (d, J=6.2 Hz, 3H), 1.28 (s, 1H), 1.01 (d, J=5.7 Hz, 3H), 0.91-0.77 (m, 10H), 0.57 (s, 3H).
Data for Isomer 3: LCMS (ESI): m/z [M+H]⁺ calc'd for C₄₉H₆₅NO₉S 927.5; found 928.4; ¹H NMR (400 MHZ, CD₃OD) δ 8.79-8.66 (m, 1H), 8.17-8.04 (m, 1H), 7.88 (dd, J=19.8, 5.4 Hz, 1H), 7.52 (dd, J=7.7, 4.8 Hz, 1H), 7.45-7.16 (m, 7H), 7.15-6.98 (m, 1H), 5.50-5.38 (m, 1H), 5.16 (d, J=8.2 Hz, 2H), 4.32 (d, J=12.0 Hz, 1H), 4.24-4.16 (m, 1H), 4.14-4.02 (m, 2H), 4.00-3.72 (m, 5H), 3.62 (dd, J=30.7, 6.5 Hz, 2H), 3.28-3.14 (m, 2H), 3.11-2.92 (m, 5H), 2.88 (d, J=6.7 Hz, 3H), 2.74-2.54 (m, 1H), 2.52-2.12 (m, 4H), 1.94-1.65 (m, 2H), 1.61-1.47 (m, 1H), 1.43 (d, J=6.3 Hz, 3H), 1.38-1.25 (m, 2H), 1.18 (t, J=6.9 Hz, 3H), 0.98-0.73 (m, 9H), 0.68 (s, 3H).
Data for Isomer 4: LCMS (ESI): m/z [M+H]⁺ calc'd for C₄₉H₆₅N₇O₉S 927.5; found 928.4; ¹H NMR (400 MHZ, CD₃OD) δ 8.79-8.61 (m, 1H), 8.21 (d, J=47.9 Hz, 1H), 7.92 (dd, J=7.7, 1.6 Hz, 1H), 7.64-7.46 (m, 2H), 7.44-7.20 (m, 5H), 7.07 (d, J=8.7 Hz, 1H), 5.84-5.45 (m, 1H), 5.26-5.02 (m, 2H), 4.42-3.38 (m, 11H), 3.27-3.06 (m, 4H), 3.05-2.94 (m, 3H), 2.93-2.70 (m, 4H), 2.53 (t, 1H), 2.27-2.09 (m, 2H), 2.01 (d, J=3.8 Hz, 1H), 1.87-1.54 (m, 3H), 1.52-1.26 (m, 3H), 1.26-0.98 (m, 4H), 0.97-0.40 (m, 12H).
Step 12. A mixture of benzyl ((2S)-1-(((6³S)-1¹-ethyl-1²-(2-((S)-1-methoxyethyl)pyridin-3-yl)-10,10-dimethyl-2¹,2¹-dioxido-5,7-dioxo-6¹,6²,6³,6⁴,6⁵,6⁶-hexahydro-1¹H-8-oxa-2(4,2)-thiomorpholina-1(5,3)-indola-6(1,3)-pyridazinacycloundecaphane-4-yl)amino)-3-methyl-1-oxobutan-2-yl)(methyl)carbamate (Isomer 1; 380 mg, 0.41 mmol), Pd/C, 50% wt with H₂O (100 mg) and NH₄Cl (220 mg, 4.1 mmol) in MeOH (10 mL), was stirred at 15° C. for 10 h. The mixture was filtered, the filtrate was concentrated under reduced pressure, the residue was diluted with sat. NaHCO₃ (20 mL) and extracted with DCM (20 mL×5). The combined organic layers was dried over Na₂SO₄, filtered and the filtrate was concentrated under reduced pressure to give (2S)-N-((6³S)-1¹-ethyl-1²-(2-((S)-1-methoxyethyl)pyridin-3-yl)-10,10-dimethyl-2¹,2¹-dioxido-5,7-dioxo-6¹,6²,6³,6⁴,6⁵,6⁶-hexahydro-1¹H-8-oxa-2(4,2)-thiomorpholina-1(5,3)-indola-6(1,3)-pyridazinacycloundecaphane-4-yl)-3-methyl-2-(methylamino) butanamide (300 mg, 92% yield) as a solid, and used in the next step without further purification. LCMS (ESI): m/z [M+H]⁺ calc'd for C₄₁H₅₉N₇O₇S 793.4; found 794.4.

A similar reaction was undertaken using Isomers 2, 3 and 4 as starting material to give the respective products.
Data for Isomer 2: Starting from (170 mg, 0.18 mmol) to give (140 mg, 98% yield). LCMS (ESI): m/z [M+H]⁺ calc'd for C₄₁H₅₉N₇O₇S 793.4; found 794.4.
Data for Isomer 3: Starting from (390 mg, 0.42 mmol) to give (300 mg, 90% yield). LCMS (ESI): m/z [M+H]⁺ calc'd for C₄₁H₅₉N₇O₇S 793.4; found 794.3.
Data for Isomer 4: Starting from (240 mg, 0.26 mmol) to give (200 mg, 96% yield). LCMS (ESI): m/z [M+H]⁺ calc'd for C₄₁H₅₉N₇O₇S 793.4; found 794.3.
Step 13. To a mixture of (2S)-N-((6³S)-1¹-ethyl-1²-(2-((S)-1-methoxyethyl)pyridin-3-yl)-10,10-dimethyl-2¹,2¹-dioxido-5,7-dioxo-6¹,6²,6³,6⁴,6⁵,6⁶-hexahydro-1¹H-8-oxa-2(4,2)-thiomorpholina-1(5,3)-indola-6(1,3)-pyridazinacycloundecaphane-4-yl)-3-methyl-2-(methylamino) butanamide (Isomer 1; 120 mg, 0.15 mmol) and (3S)-1-{3-[(formyloxy)methyl]phenyl}pyrrolidine-3-carboxylic acid (56 mg, 0.23 mmol) in DMF (5 mL) at 0° C. was added DIPEA (390 mg, 3 mmol) and HATU (87 mg, 0.23 mmol). The mixture was stirred at 0° C. for 1 h, then diluted with EtOAc (20 mL) and washed with H₂O (20 mL×2). The organic layer was dried over Na₂SO₄, filtered, the filtrate was concentrated under reduced pressure and the residue was purified by silica gel chromatography to give benzyl (3S)-3-(((2S)-1-(((6³S)-1¹-ethyl-1²-(2-((S)-1-methoxyethyl)pyridin-3-yl)-10,10-dimethyl-2¹,2¹-dioxido-5,7-dioxo-6¹,6²,6³,6⁴,6⁵,6⁶-hexahydro-1¹H-8-oxa-2(4,2)-thiomorpholina-1(5,3)-indola-6(1,3)-pyridazinacycloundecaphane-4-yl)amino)-3-methyl-1-oxobutan-2-yl)(methyl)carbamoyl)pyrrolidine-1-carboxylate (111 mg, 72% yield) as a solid. LCMS (ESI): m/z [M+H]⁺ calc'd for C₅₄H₇₂N₈O₁₀S 1024.5; found 1025.3.

A similar reaction was undertaken using Isomers 2, 3 and 4 as starting material to give the respective products.
Data Isomer 2: Starting from (150 mg, 0.19 mmol) to give (120 mg, 62% yield). LCMS (ESI): m/z [M+H]⁺ calc'd for C₅₄H₇₂N₈O₁₀S 1024.5; found 1025.4.
Data for Isomer 3: Starting from (300 mg, 0.38 mmol) to give (300 mg, 77% yield). LCMS (ESI): m/z [M+H]⁺ calc'd for C₅₄H₇₂N₈O₁₀S 1024.5; found 1025.5.
Data for Isomer 4: Starting from (199 mg, 0.25 mmol) to give (220 mg, 85% yield). LCMS (ESI): m/z [M+H]⁺ calc'd for C₅₄H₇₂N₈O₁₀S 1024.5; found 1025.4.
Step 14. A mixture of benzyl (3S)-3-(((2S)-1-(((6³S)-1¹-ethyl-1²-(2-((S)-1-methoxyethyl)pyridin-3-yl)-10,10-dimethyl-2¹,2¹-dioxido-5,7-dioxo-6¹,6²,6³,6⁴,6⁵,6⁶-hexahydro-1¹H-8-oxa-2(4,2)-thiomorpholina-1(5,3)-indola-6(1,3)-pyridazinacycloundecaphane-4-yl)amino)-3-methyl-1-oxobutan-2-yl)(methyl)carbamoyl)pyrrolidine-1-carboxylate (Isomer 1; 111 mg, 0.11 mmol), Pd/C, 50% wt. with H₂O (30 mg) and NH₄Cl (60 mg, 1.1 mmol) in MeOH (20 mL) was stirred at 15° C. for 10 h. The mixture was filtered, the filtrate was concentrated under reduced pressure and the residue was diluted with DCM (20 mL) and washed with sat. NaHCO₃. The organic layer was dried over Na₂SO₄, filtered and the filtrate was concentrated under reduced pressure to give (3S)-N-((2S)-1-(((6³S)-1¹-ethyl-1²-(2-((S)-1-methoxyethyl)pyridin-3-yl)-10,10-dimethyl-2¹,2¹-dioxido-5,7-dioxo-6¹,6²,6³,6⁴,6⁵,6⁶-hexahydro-1¹H-8-oxa-2 (4,2)-thiomorpholina-1(5,3)-indola-6(1,3)-pyridazinacycloundecaphane-4-yl)amino)-3-methyl-1-oxobutan-2-yl)-N-methylpyrrolidine-3-carboxamide (77 mg, 79% yield) as a solid, which was used in the next step without further purification. LCMS (ESI): m/z [M+H]⁺ calc'd for C₄₆H₆₆N₈O₈S 890.5; found 891.4.

A similar reaction was undertaken using Isomers 2, 3 and 4 as starting material to give the respective products.
Data for Isomer 2: Starting from (120 mg, 0.12 mmol) to give (85 mg, 89% yield). LCMS (ESI): m/z [M+H]⁺ calc'd for C₄₆H₆₆N₈O₈S 890.5; found 891.4.
Data for Isomer 3: Starting from (300 mg, 0.34 mmol) to give (220 mg, 73% yield). LCMS (ESI): m/z [M+H]⁺ calc'd for C₄₆H₆₆N₈O₈S 890.5; found 891.5.
Data for Isomer 4: Starting from (220 mg, 0.21 mmol) to give (147 mg, 71% yield). LCMS (ESI): m/z [M+H]⁺ calc'd for C₄₆H₆₆N₈O₈S 890.5; found 891.4.
Step 15. To a mixture of (3S)-N-((2S)-1-(((6³S)-1¹-ethyl-1²-(2-((S)-1-methoxyethyl)pyridin-3-yl)-10,10-dimethyl-2¹,2¹-dioxido-5,7-dioxo-6¹,6²,6³,6⁴,6⁵,6⁶-hexahydro-1¹H-8-oxa-2(4,2)-thiomorpholina-1(5,3)-indola-6(1,3)-pyridazinacycloundecaphane-4-yl)amino)-3-methyl-1-oxobutan-2-yl)-N-methylpyrrolidine-3-carboxamide (Isomer 1; 77 mg, 0.086 mmol) in DCM (2 mL) at 0° C. was added sat. NaHCO₃ (2 mL) and prop-2-enoyl chloride (7 mg, 0.077 mmol) in DCM (1 mL). The mixture was stirred at 0° C. for 30 min, then H₂O added, and the mixture was extracted with DCM (10 mL×3). The combined organic layers were dried over Na2SO4, filtered, the filtrate was concentrated under reduced pressure and the residue was purified by preparative-TLC to give (3S)-1-acryloyl-N-((2S)-1-(((6³S)-1¹-ethyl-1²-(2-((S)-1-methoxyethyl)pyridin-3-yl)-10,10-dimethyl-2¹,2¹-dioxido-5,7-dioxo-6¹,6²,6³,6⁴,6⁵,6⁶-hexahydro-1¹H-8-oxa-2(4,2)-thiomorpholina-1(5,3)-indola-6(1,3)-pyridazinacycloundecaphane-4-yl)amino)-3-methyl-1-oxobutan-2-yl)-N-methylpyrrolidine-3-carboxamide (23 mg, 28% yield) as a solid. LCMS (ESI): m/z [M+H]⁺ calc'd for C₄₉H₆₈N₈O₉S 944.5; found 945.4; ¹H NMR (400 MHZ, CD₃OD) δ 8.75-8.74 (m, 1H), 7.92-7.90 (m, 1H), 7.54-7.51 (m, 1H), 7.43 (dd, J=8.8, 2.2 Hz, 1H), 7.34 (d, J=3.2 Hz, 1H), 7.25-7.15 (m, 1H), 6.71-6.60 (m, 1H), 6.32-6.25 (m, 1H), 5.77 (dd, J=10.5, 1.9 Hz, 1H), 5.53-5.48 (m, 1H), 4.62 (dd, J=24.9, 11.1 Hz, 1H), 4.45 (s, 1H), 4.13-4.03 (m, 3H), 3.89-3.76 (m, 6H), 3.69-3.63 (m, 2H), 3.60-3.35 (m, 3H), 3.25-3.21 (m, 3H), 3.13-3.11 (m, 1H), 3.00 (d, J=2.3 Hz, 5H), 2.90 (d, J=3.5 Hz, 2H), 2.25-2.20 (m, 2H), 2.16-2.09 (m, 3H), 2.04-1.94 (m, 2H), 1.80-1.72 (m, 2H), 1.46-1.43 (m, 3H), 1.29 (m, 3H), 1.26-1.22 (m, 3H), 1.01-0.98 (m, 3H), 0.95-0.88 (m, 3H), 0.84-0.81 (m, 3H), 0.62-0.59 (m, 2H).

A similar reaction was undertaken using Isomers 2, 3 and 4 as starting material to give the respective products.
Data for Isomer 2: Starting from (110 mg, 0.12 mmol) to give (24.5 mg, 21% yield). LCMS (ESI): m/z [M+H]⁺ calc'd for C₄₉H₆₈N₈O₉S 944.5; found 945.3; ¹H NMR (400 MHZ, CD₃OD) δ 8.71 (dd, J=4.8, 1.7 Hz, 1H), 7.91-7.78 (m, 1H), 7.52 (dd, J=7.7, 4.9 Hz, 1H), 7.45-7.36 (m, 1H), 7.25-7.03 (m, 2H), 6.65-6.56 (m, 1H), 6.30-6.22 (m, 1H), 5.76-5.70 (m, 1H), 5.67-5.48 (m, 1H), 5.27 (dd, J=11.7, 8.2 Hz, 1H), 4.69 (dd, J=10.9, 3.3 Hz, 1H), 4.37-4.28 (m, 1H), 4.26-4.18 (m, 1H), 4.18-3.98 (m, 3H), 3.97-3.83 (m, 4H), 3.82-3.62 (m, 4H), 3.60-3.41 (m, 3H), 3.28-3.20 (m, 2H), 3.14 (d, J=10.4 Hz, 3H), 3.06 (d, J=4.8 Hz, 3H), 2.96 (s, 1H), 2.89-2.77 (m, 1H), 2.73-2.55 (m, 1H), 2.48-2.34 (m, 1H), 2.33-2.18 (m, 3H), 2.13-1.95 (m, 2H), 1.90-1.84 (m, 1H), 1.80-1.67 (m, 2H), 1.43 (m, 3H), 1.27 (s, 1H), 1.14-0.95 (m, 4H), 0.94-0.85 (m, 4H), 0.82 (d, J=6.2 Hz, 5H), 0.56 (d, J=8.7 Hz, 3H).
Data for Isomer 3: Starting from (120 mg, 0.13 mmol) to give (32 mg, 11% yield). LCMS (ESI): m/z [M+H]⁺ calc'd for C₄₉H₆₈N₈O₉S 944.5; found 945.5; ¹H NMR (400 MHZ, CD₃OD) δ 8.73 (dt, J=3.8, 1.9 Hz, 1H), 7.93-7.86 (m, 1H), 7.53 (dd, J=7.7, 4.9 Hz, 1H), 7.40 (dd, J=8.8, 2.3 Hz, 1H), 7.28 (d, J=9.6 Hz, 1H), 7.13-6.99 (m, 1H), 6.65 (ddd, J=35.6, 16.8, 10.5 Hz, 1H), 6.28 (ddd, J=16.8, 4.9, 1.9 Hz, 1H), 5.75 (td, J=10.4, 1.9 Hz, 1H), 5.53-5.34 (m, 1H), 4.63 (dd, J=13.4, 11.3 Hz, 1H), 4.26 (d, J=11.1 Hz, 1H), 4.12-4.01 (m, 2H), 4.00-3.82 (m, 5H), 3.82-3.45 (m, 7H), 3.41-3.33 (m, 1H), 3.14-3.02 (m, 4H), 3.02-2.87 (m, 5H), 2.62-2.34 (m, 3H), 2.33-2.17 (m, 3H), 2.10-1.94 (m, 1H), 1.69-1.52 (m, 1H), 1.46-1.39 (m, 3H), 1.27 (s, 2H), 1.23-1.16 (m, 3H), 1.16-1.01 (m, 2H), 0.96-0.90 (m, 3H), 0.88-0.74 (m, 6H), 0.73-0.63 (m, 3H).
Data for Isomer 4: Starting from (147 mg, 0.16 mmol) to give (47.2 mg, 31% yield). LCMS (ESI): m/z [M+H]⁺ calc'd for C₄₉H₆₈N₈O₉S 944.5; found 945.3; ¹H NMR (400 MHZ, CD₃OD) δ 8.73-8.72 (m, 1H), 7.92 (dd, J=7.8, 1.6 Hz, 1H), 7.53-7.50 (m, 1H), 7.49-7.46 (m, 1H), 7.41-7.38 (m, 1H), 7.07 (d, J=8.8 Hz, 1H), 6.65-6.56 (m, 1H), 6.28-6.23 (m, 1H), 5.76-5.71 (m, 2H), 4.59-4.55 (m, 1H), 4.34-4.30 (m, 1H), 4.13-4.03 (m, 4H), 3.88-3.72 (m, 6H), 3.68-3.48 (m, 5H), 3.30-3.20 (m, 4H), 3.08-3.07 (m, 3H), 3.02 (d, J=4.1 Hz, 4H), 2.55-2.53 (m, 1H), 2.34-2.19 (m, 3H), 2.11-2.00 (m, 3H), 1.90-1.88 (m, 1H), 1.76-1.74 (m, 2H), 1.44 (d, J=6.3 Hz, 3H), 1.29 (s, 1H), 1.23-1.20 (m, 3H), 0.91-0.86 (m, 3H), 0.78-0.75 (m, 5H), 0.69-0.66 (m, 3H).

### Example 11. Synthesis of 3-acryloyl-N-((2S)-1-(((22 S,63S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)amino)-3-methyl-1-oxobutan-2-yl)-N,1-dimethyl-1,3,8-triazaspiro[4.5]decane-8-carboxamide (A199)

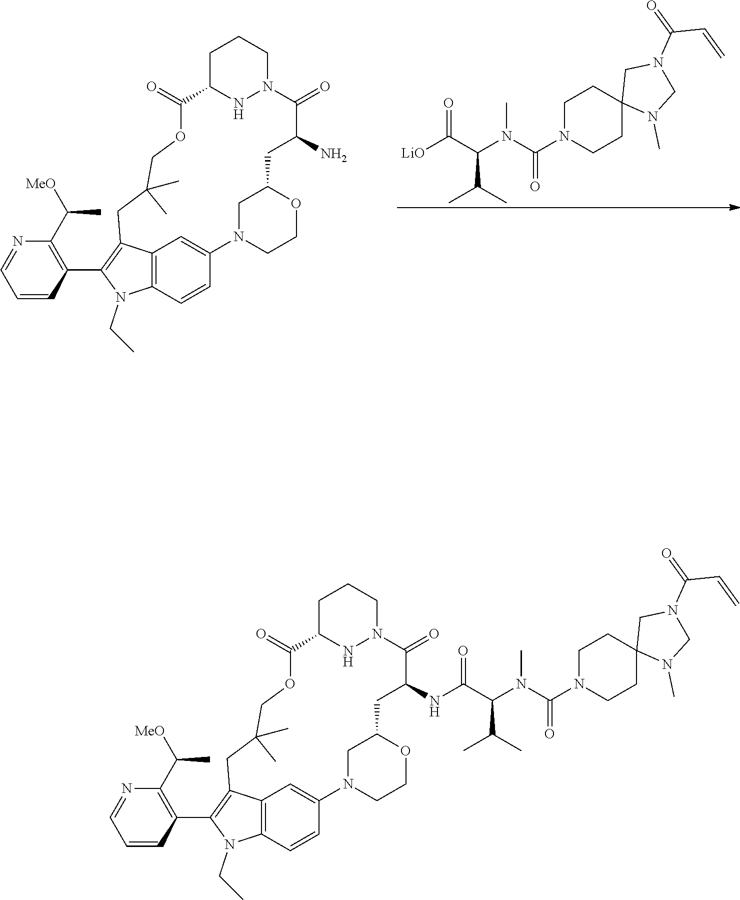

Step 1. To a mixture of (2²S,6³S,4S)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-2(4,2)-morpholina-1(5,3)-indola-6(1,3)-pyridazinacycloundecaphane-5,7-dione (150 mg, 0.24 mmol) and (2S)-3-methyl-2-{methyl[1-methyl-3-(prop-2-enoyl)-1,3,8-triazaspiro[4.5]decan-8-yl]carbonylamino]butanoate, lithium salt (132 mg, 0.36 mmol) in DMF (5 mL) at 0° C. was added HATU (108 mg, 0.28 mmol) and DIPEA (459 mg, 3.5 mmol). The mixture was stirred at 0° C. for 1 h, then diluted with EtOAc (30 mL), washed with H₂O (10 mL×2) and brine (10 mL). The organic layer was dried over Na₂SO₄ and filtered. The filtrate was concentrated under reduced pressure and the crude residue was purified by silica gel column chromatography and preparative-HPLC to give 3-acryloyl-N-((2S)-1-(((2²S,6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)amino)-3-methyl-1-oxobutan-2-yl)-N, 1-dimethyl-1,3,8-triazaspiro[4.5]decane-8-carboxamide (6.9 mg, 3% yield) as a solid. LCMS (ESI): m/z [M+H]⁺ calc'd for C₅₃H₇₆N₁₀O₈ 980.6; found 367.2; ¹H NMR (400 MHZ, CD₃OD) δ8.71 (dd, J=4.8, 1.6 Hz, 1H), 7.86 (dd, J=7.8, 1.6 Hz, 1H), 7.51 (dd, J=7.8, 4.8 Hz, 1H), 7.39 (d, J=8.8 Hz, 1H), 7.14-7.04 (m, 2H), 6.67-6.44 (m, 1H), 6.31 (d, J=16.8 Hz, 1H), 5.81-5.75 (m, 1H), 5.65 (d, J=9.0 Hz, 1H), 4.51-4.13 (m, 2H), 4.33 (s, 1H), 4.27-4.18 (m, 1H), 4.17-4.08 (m, 1H), 3.96-3.87 (m, 3H), 3.87-3.77 (m, 3H), 3.76-3.65 (m, 4H), 3.64-3.51 (m, 3H), 3.28-3.24 (m, 1H), 3.16 (s, 3H), 3.10-3.02 (m, 1H), 2.99-2.90 (m, 2H), 2.87-2.74 (m, 5H), 2.70-2.53 (m, 2H), 2.40-2.30 (m, 3H), 2.27-2.18 (m, 1H), 2.14-2.05 (m, 2H), 1.98-1.88 (m, 3H), 1.79-1.68 (m, 2H), 1.65-1.47 (m, 3H), 1.44 (d, J=6.4 Hz, 3H), 1.04 (t, J=6.8 Hz, 3H), 0.95 (d, J=6.4 Hz, 3H), 0.88 (d, J=6.4 Hz, 3H), 0.80-0.60 (m, 6H).

### Example 12. Synthesis 2-acryloyl-N-((2S)-1-(((6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-2¹,2²,2³,2⁶,6¹,6²,6³,6⁴,6⁵,6⁶-decahydro-1¹H-8-oxa-1(5,3)-indola-6(1,3)-pyridazina-2(5,1)- pyridinacycloundecaphane-4-yl)amino)-3-methyl-1-oxobutan-2-yl)-N-methyl-2,8-diazaspiro[4.5]decane-8-carboxamide (A83)

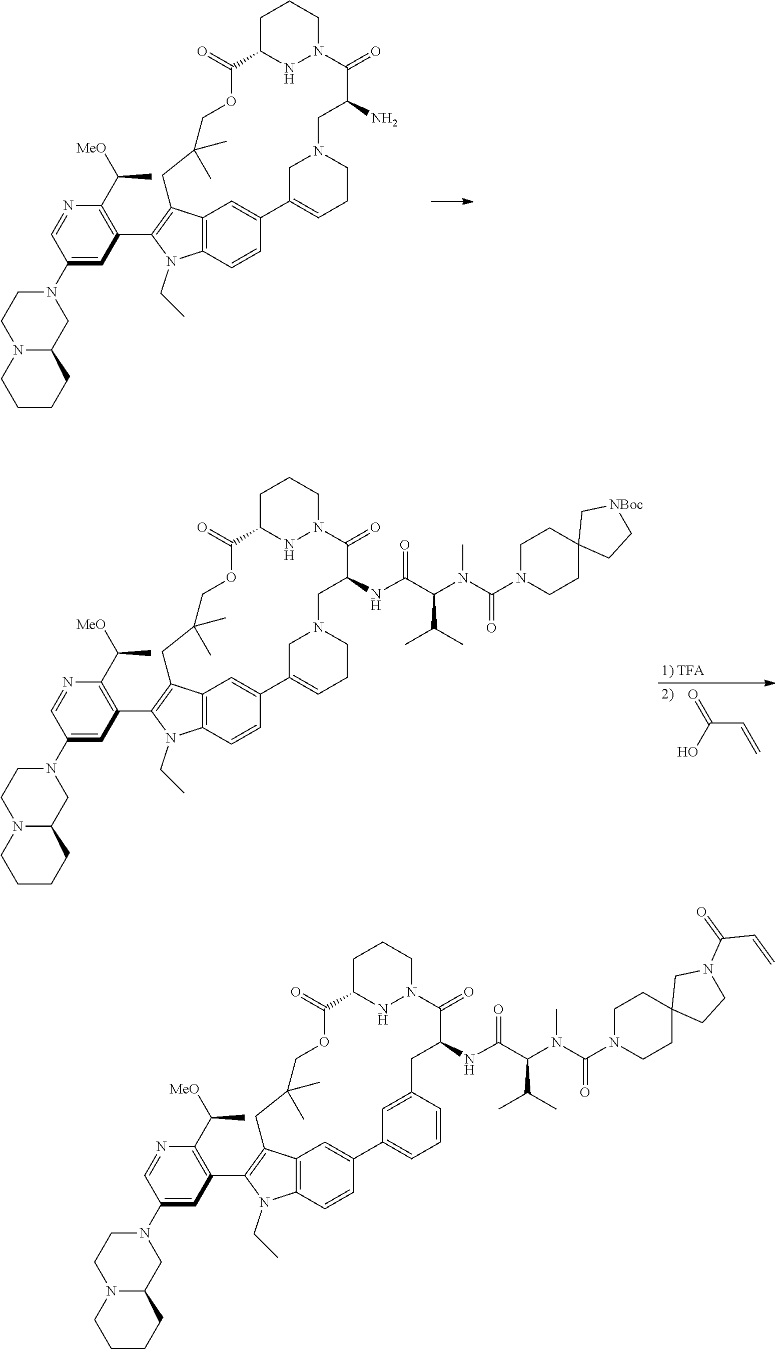

Step 1. To a mixture of (6³S,4S)-4-amino-1¹-ethyl-1²-(2-((S)-1-methoxyethyl)-5-((R)-octahydro-2H-pyrido[1,2-a]pyrazin-2-yl)pyridin-3-yl)-10,10-dimethyl-2¹,2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-5,7-dione (150 mg, 0.23 mmol) in DMF (2 mL) at 0° C. was added (2S)-2-({2-[(tert-butoxy)carbonyl]-2,8-diazaspiro[4.5]decan-8-yl}carbonyl (methyl)amino)-3-methylbutanoic acid (125 mg, 0.30 mmol), DIPEA (310 mg, 2.34 mmol) and HATU (134 mg, 0.35 mmol). The mixture was stirred at 0° C. for 1 h, then H₂O (150 mL) and extracted with EtOAc (150 mL×2). The combined organic layers were washed with H₂O (50 mL), brine (50 mL), dried over anhydrous Na₂SO₄ and filtered. The filtrate was concentrated under reduced pressure and the crude residue was purified by preparative-TLC to give tert-butyl 8-(((2S)-1-(((6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-2¹,2²,2³,2⁶,6¹,6²,6³,6⁴,6⁵,6⁶-decahydro-1¹H-8-oxa-1(5,3)-indola-6(1,3)-pyridazina-2(5, 1)-pyridinacycloundecaphane-4-yl)amino)-3-methyl-1-oxobutan-2-yl)(methyl)carbamoyl)-2,8-diazaspiro[4.5]decane-2-carboxylate (130 mg, 40% yield) as a solid. LCMS (ESI): m/z [M+H]⁺ calc'd for C₆₄H₉₅N₁₁O₈ 1145.7; found 1146.7.
Step 2. To a mixture of tert-butyl 8-(((2S)-1-(((6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-2¹,2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-4-yl)amino)-3-methyl-1-oxobutan-2-yl)(methyl)carbamoyl)-2,8-diazaspiro[4.5]decane-2-carboxylate (130 mg, 0.12 mmol) in DCM (1.0 mL) at 0° C. was added TFA (0.5 mL). The mixture was stirred at 0° C. for 1 h, then diluted with DCM (5 mL) and saturated NaHCO₃ added to adjust pH ˜9. Prop-2-enoyl chloride (10 mg, 0.11 mmol) in DCM was added at 0° C., and the mixture was stirred at 0° C. for 15 min. The mixture was poured into H₂O (50 mL) and extracted with DCM (150 mL×2). The combined organic layers were washed with H₂O (50 mL), brine (50 mL), dried over anhydrous Na₂SO₄ and filtered. The filtrate was concentrated under reduced pressure and the crude residue was purified by preparative-TLC to give 2-acryloyl-N-((2S)-1-(((6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-2¹,2²,2³,2⁶,6¹,6²,6³,6⁴,6⁵,6⁶-decahydro-1¹H-8-oxa-1(5,3)-indola-6(1,3)-pyridazina-2(5,1)- pyridinacycloundecaphane-4-yl)amino)-3-methyl-1-oxobutan-2-yl)-N-methyl-2,8-diazaspiro[4.5]decane-8-carboxamide as a solid. LCMS (ESI): m/z [M+H]⁺ calc'd for C₆₂H₈₉N₁₁O₇ 1099.7; found 1100.6; ¹H NMR (400 MHz, CD₃OD) δ 8.45 (d, J=2.8 Hz, 1H), 7.54 (d, J=9.2 Hz, 2H), 7.42 (d, J=8.4 Hz, 2H), 6.65 (m, 1H), 6.41-6.21 (m, 2H), 5.93 (dd, J=7.6, 3.8 Hz, 1H), 5.81-5.75 (m, 1H), 4.50 (d, J=12.8 Hz, 1H), 4.20-4.04 (m, 3H), 3.98-3.71 (m, 8H), 3.63-3.48 (m, 2H), 3.46-3.36 (m, 2H), 3.30-3.15 (m, 3H), 3.12-2.97 (m, 6H), 2.93-2.76 (m, 6H), 2.64 (t, J=11.2 Hz, 2H), 2.55 (d, J=11.6 Hz, 9H), 2.45-2.12 (m, 4H), 1.99-1.83 (m, 2H), 1.80-1.55 (m, 10H), 1.48-1.29 (m, 6H), 1.22 (t, J=7.0 Hz, 3H), 0.93 (dd, J=22.8, 6.4 Hz, 9H), 0.72 (s, 3H).

### Example 13. Synthesis of 3-acryloyl-N-((2S)-1-(((2²S,6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)amino)-3-methyl-1-oxobutan-2-yl)-N-methyl-1-oxa-3,8-diazaspiro[4.5]decane-8-carboxamide (A20)

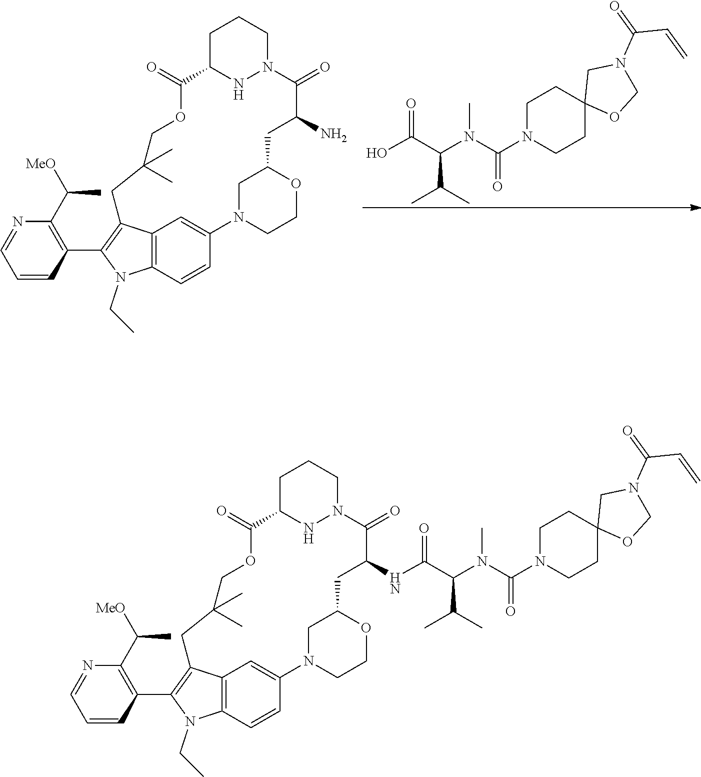

Step 1. To a mixture of (2²S,6³S,4S)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-2(4,2)-morpholina-1(5,3)-indola-6(1,3)-pyridazinacycloundecaphane-5,7-dione (450 mg, 0.7 mmol). The mixture was stirred at 0° C. for 1 h, then H₂O (20 mL) was added, and the mixture was extracted with EtOAc (30 mL×3). The combined organic layers were washed with brine (20 mL), dried over anhydrous Na₂SO₄, and filtered. The filtrate was concentrated under reduced pressure and the crude residue was purified by silica gel column chromatography and preparative-HPLC to give 3-acryloyl-N-((2S)-1-(((2²S,6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-morpholina-1(5,3)-indola-6(1,3)- pyridazinacycloundecaphane-4-yl)amino)-3-methyl-1-oxobutan-2-yl)-N-methyl-1-oxa-3,8-diazaspiro[4.5]decane-8-carboxamide (297 mg, 40% yield) as a solid. LCMS (ESI): m/z [M+H]⁺ calc'd for C₅₂H₇₃N₉O₉ 967.6; found 968.6; ¹H NMR (400 MHZ, CD₃OD) δ 8.72-8.69 (m, 1H), 8.10 (d, J=6.4 Hz, 1H), 7.89-7.80 (m, 1H), 7.56-7.47 (m, 1H), 7.45-7.35 (m, 1H), 7.17-7.01 (m, 2H), 6.62-6.45 (m, 1H), 6.32 (s, 1H), 5.85-5.71 (m, 1H), 5.64 (d, J=8.8 Hz, 1H), 5.19 (s, 1H), 5.10 (s, 1H), 4.46 (d, J=12.4 Hz, 1H), 4.25-4.03 (m, 2H), 3.99-3.61 (m, 8H), 3.61-3.33 (m, 6H), 3.29-3.18 (m, 2H), 3.15 (s, 3H), 2.99-2.71 (m, 6H), 2.68-2.46 (m, 2H), 2.30-2.17 (m, 1H), 2.12-2.02 (m, 2H), 1.96-1.54 (m, 8H), 1.43 (d, J=6.4 Hz, 3H), 1.15-0.97 (m, 3H), 0.96-0.79 (m, 6H), 0.77-0.53 (m, 6H).

### Example 14. Synthesis of 4-acryloyl-N-((2S)-1-(((6³S,4S)-1¹-ethyl-1²-(2-((S)-1-methoxyethyl)pyridin-3-yl)-10,10-dimethyl-5,7-dioxo-2¹,2²,2³,2⁶,6¹,6²,6³,6⁴,6⁵,6⁶-decahydro-1¹H-8-oxa-1(5,3)-indola-6(1,3)-pyridazina- 2(5,1)-pyridinacycloundecaphane-4-yl)amino)-3-methyl-1-oxobutan-2-yl)-N-methyl-1-propyl-1,4,9-triazaspiro[5.5]undecane-9-carboxamide (A54)

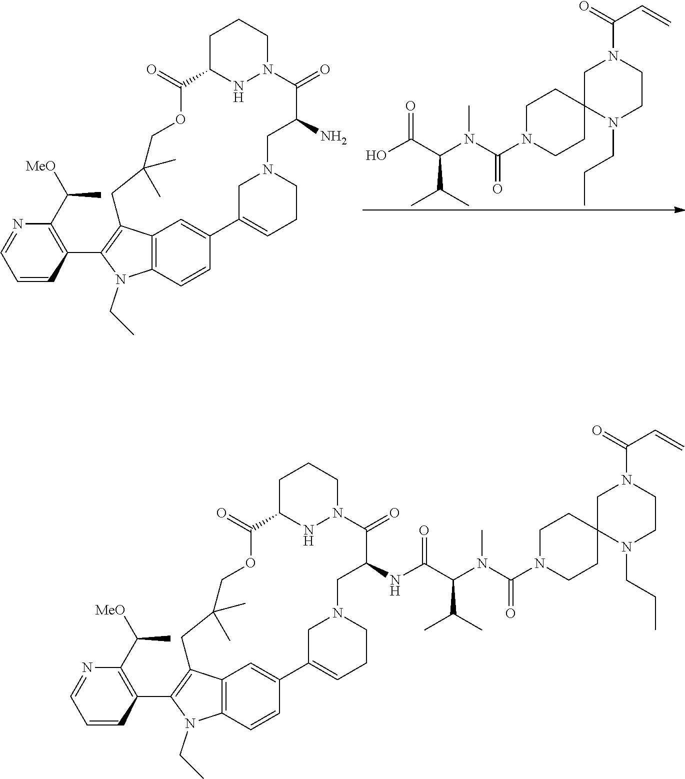

Step 1. To a mixture of (6³S,4S)-4-amino-1¹-ethyl-1²-(2-((S)-1-methoxyethyl)pyridin-3-yl)-10,10-dimethyl-2¹,2²,2³,2⁶,6¹,6²,6³,6⁴,6⁵,6⁶-decahydro-1¹H-8-oxa-1(5,3)-indola-6(1,3)-pyridazina-2(5,1)- pyridinacycloundecaphane-5,7-dione (113 mg, 0.18 mmol) and (2S)-3-methyl-2-{methyl[4-(prop-2-enoyl)-1-propyl-1,4,9-triazaspiro[5.5]undecan-9-yl]carbonylamino}butanoic acid, lithium salt (88 mg, 0.22 mmol) in DMF (2 mL) at 0° C. was added DIPEA (464 mg, 3.6 mmol) and HATU (82 mg, 0.23 mmol). The mixture was stirred at 0° C. for 1 h, then H₂O (20 mL) was added, and the mixture was extracted with DCM (20 mL×3). The combined organic layers were washed with brine (20 mL), dried over anhydrous Na₂SO₄, and filtered. The filtrate was concentrated under reduced pressure and the crude residue was purified by preparative-HPLC to give 4-acryloyl-N-((2S)-1-(((6³S,4S)-1¹-ethyl-1²-(2-((S)-1-methoxyethyl)pyridin-3-yl)-10,10-dimethyl-5,7-dioxo-2¹,2²,2³,2⁶,6¹,6²,6³,6⁴,6⁵,6⁶-decahydro-1¹H-8-oxa-1(5,3)-indola-6(1,3)-pyridazina-2(5,1)- pyridinacycloundecaphane-4-yl)amino)-3-methyl-1-oxobutan-2-yl)-N-methyl-1-propyl-1,4,9-triazaspiro[5.5]undecane-9-carboxamide (26 mg, 14% yield) as a solid. LCMS (ESI): m/z [M+H]⁺ calc'd for C₅₇H₈₂N₁₀O₇ 1018.6; found 1019.6; ¹H NMR (400 MHZ, CD₃OD) δ 8.73 (dd, J=8.0, 4.0 Hz, 1H), 7.90 (dd, J=8.0, 4.0 Hz, 1H), 7.54-7.51 (m, 3H), 7.41-7.38 (m, 1H), 6.90-6.74 (m, 1H), 6.30-6.18 (m, 2H), 5.91-5.88 (m, 1H), 5.80-5.75 (m, 1H), 4.59-4.46 (m, 1H), 4.10-3.47 (m, 15H), 3.19-2.72 (m, 17H), 2.42-2.15 (m, 8H), 2.08-1.63 (m, 7H), 1.48-1.44 (m, 6H), 1.16 (t, J=6.4 Hz, 3H), 0.93-0.86 (m, 9H), 0.66 (s, 3H).

### Example 15. Synthesis of 4-acryloyl-N-((2S)-1-(((6³S,4S)-1¹-ethyl-2⁴-fluoro-1²-(2-((S)-1-methoxyethyl)pyridin-3-yl)-10,10-dimethyl-5,7-dioxo-2¹,2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-4-yl)amino)-3-methyl-1-oxobutan-2-yl)-N-methyl-1-oxa-4,9-diazaspiro[5.5]undecane-9-carboxamide (A335)

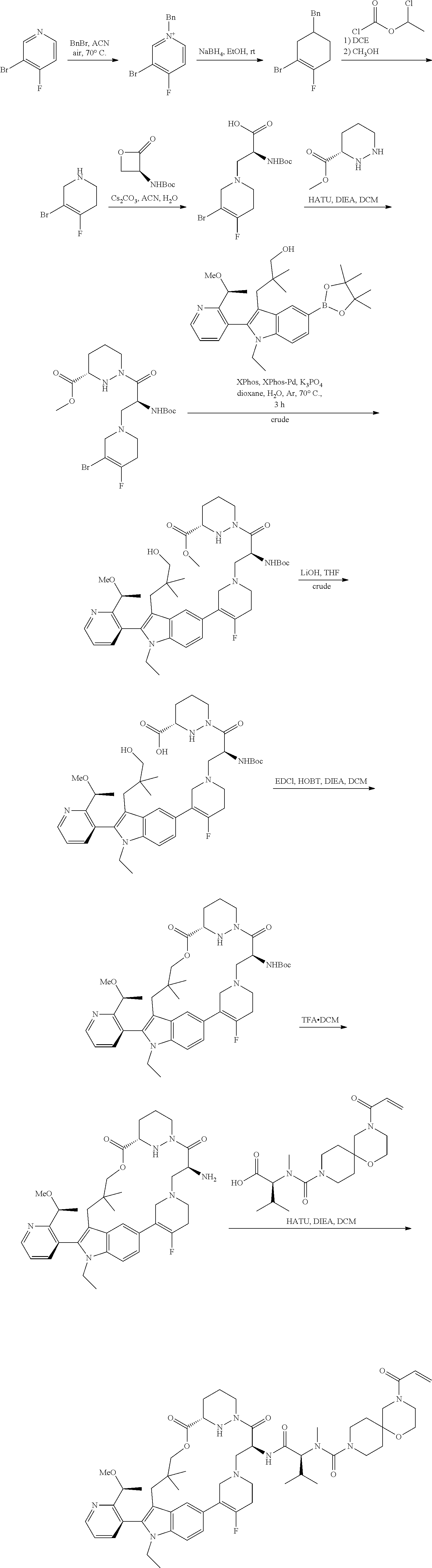

Step 1. Into a 100 mL round-bottom flask were added 3-bromo-4-fluoropyridine (9 g, 1 equiv), 100 mL ACN and BnBr (10.0 g, 1.2 equiv) at room temperature. The resulting mixture was stirred for 4 h at 70° C. under air atmosphere. The residue was washed with acetone (3×5 mL) to afford 1-benzyl-3-bromo-4-fluoro-pyridine (13 g, 85% yield) as a white solid. ESI-MS m/z=266.0, 268.0 [M]⁺; Calculated MW: 266.0.
Step 2. Into a 500 mL round-bottom flask were added 1-benzyl-3-bromo-4-fluoro-pyridine (13 g, 48.7 mmol, 1.0 equiv), EtOH (200 mL) and NaBH₄ (7.36 g, 194.668 mmol, 4 equiv) at 0° C. The resulting mixture was stirred for 4 h at 0° C. under air atmosphere. The reaction was quenched with Water/Ice at 0° C. The resulting mixture was concentrated under reduced pressure and the remaining residue was extracted with EtOAc (3×30 mL). The combined organic layers were dried over anhydrous Na₂SO₄. After filtration, the filtrate was concentrated under reduced pressure to afford 1-benzyl-3-bromo-4-fluoro-5,6-dihydro-2H-pyridine (8 g, 61% yield) as an oil. ESI-MS m/z=270.1, 272.1 [M+H]⁺; Calculated MW: 269.0.
Step 3. Into a 100 mL round-bottom flask were added 1-benzyl-3-bromo-4-fluoro-5,6-dihydro-2H-pyridine (3000 mg, 11.1 mmol, 1.0 equiv) and chloroethyl chloroformate (3175.2 mg, 22.2 mmol, 2 equiv) at 0° C. The resulting mixture was stirred overnight at 80° C. under air atmosphere. The resulting mixture was concentrated under reduced pressure. Then CH₃OH (27.3 mL, 673.6 mmol, 60.7 equiv) was added at room temperature. The resulting mixture was stirred for 5 h at 60° C. under air atmosphere. The mixture was acidified to pH 2 with HCl (dioxane). The precipitated solids were collected by filtration and washed with acetonitrile (3×5 mL) to afford 3-bromo-4-fluoro-1,2,5,6-tetrahydropyridine (750 mg, 37% yield) as an off-white solid. ESI-MS m/z=180.1, 182.1 [M+H]⁺; Calculated MW: 179.0.
Step 4. Into a 40 mL vial were added 3-bromo-4-fluoro-1,2,5,6-tetrahydropyridine (500 mg, 2.8 mmol, 1.0 equiv), tert-butyl N-[(3S)-2-oxooxetan-3-yl]carbamate (779.89 mg, 4.165 mmol, 1.5 equiv), ACN (5 mL), Cs₂CO₃ (2262.4 mg, 6.9 mmol, 2.5 equiv), H₂O (5 mL) at room temperature. The resulting mixture was stirred for 2 h at 40° C. under air atmosphere. The resulting mixture was concentrated under reduced pressure. The crude product was purified by reverse phase flash with the following conditions to afford (2S)-3-(3-bromo-4-fluoro-5,6-dihydro-2H-pyridin-1-yl)-2-[(tert-butoxycarbonyl)amino]propanoic acid (900 mg, 44% yield) as an oil. ESI-MS m/z=367.0, 369.0 [M+H]⁺; Calculated MW: 366.1.
Step 5. Into a 100 mL round-bottom flask were added (2S)-3-(3-bromo-4-fluoro-5,6-dihydro-2H-pyridin-1-yl)-2-[(tert-butoxycarbonyl)amino]propanoic acid (900 mg, 2.5 mmol, 1.0 equiv), methyl (3S)-1,2-diazinane-3-carboxylate (424 mg, 2.9 mmol, 1.2 equiv), DCM (20 mL), DIEA (3167.6 mg, 24.5 mmol, 10 equiv) and HATU (1164.9 mg, 3.1 mmol, 1.25 equiv) at 0° C. The resulting mixture was stirred for 2 h at 0° C. under air atmosphere. The reaction was quenched with water at 0° C. The resulting mixture was extracted with CH₂Cl₂ (3×10 mL). The combined organic layers were dried over anhydrous Na₂SO₄. After filtration, the filtrate was concentrated under reduced pressure. The residue was purified by silica gel column chromatography to afford methyl (3S)-1-[(2S)-3-(3-bromo-4-fluoro-3,6-dihydro-2H-pyridin-1-yl)-2-[(tert-butoxycarbonyl)amino]propanoyl]-1,2-diazinane-3-carboxylate (390 mg, 32% yield) as an oil. ESI-MS m/z=493.0, 495.0 [M+H]⁺; Calculated MW: 492.1.
Step 6. Into a 50 mL sealed tube were added 3-[(2M)-5-(4,5-dimethyl-1,3,2-dioxaborolan-2-yl)-1-ethyl-2-{2-[(1S)-1-methoxyethyl]pyridin-3-yl}indol-3-yl]-2,2-dimethylpropan-1-ol (395 mg, 0.85 mmol, 1.20 equiv), methyl (3S)-1-[(2S)-3-(3-bromo-4-fluoro-5,6-dihydro-2H-pyridin-1-yl)-2-[(tert-butoxycarbonyl)amino]propanoyl]-1,2-diazinane-3-carboxylate (350 mg, 0.71 mmol, 1.0 equiv), K₃PO₄ (451.8 mg, 2.1 mmol, 3 equiv), X-Phos (67.6 mg, 0.14 mmol, 0.2 equiv), 3rd Generation XPhos precatalyst (120.1 mg, 0.14 mmol, 0.2 equiv), dioxane (10 mL) and H₂O (2 mL) at room temperature. The resulting mixture was stirred for 3 h at 70° C. under argon atmosphere. The reaction was quenched with water at room temperature. The resulting mixture was extracted with EtOAc (3×10 mL). The combined organic layers were dried over anhydrous Na₂SO₄. After filtration, the filtrate was concentrated under reduced pressure. The crude (340 mg) product resulting mixture was used in the next step directly without further purification. ESI-MS m/z=779.6 [M+H]⁺; Calculated MW: 778.4.
Step 7. Into a 100 mL round-bottom flask were added (2S)-3-(3-bromo-4-fluoro-5,6-dihydro-2H-pyridin-1-yl)-2-[(tert-butoxycarbonyl)amino]propanoic acid (500 mg, 1.0 equiv), THF (5 mL) and LiOH (5 equiv) at 0° C. The resulting mixture was stirred for 2 h at 0° C. under air atmosphere. The reaction was quenched with water at room temperature. The resulting mixture was extracted with CH₂Cl₂ (3×10 mL). The combined water layers were acidified to pH 5 with conc. HCl and the resulting mixture was extracted with CH₂Cl₂ (3×30 mL). The combined organic layers were dried over anhydrous Na₂SO₄. After filtration, the filtrate was concentrated under reduced pressure to afford 340 mg (3S)-1-[(2S)-2-[(tert-butoxycarbonyl)amino]-3-{3-[(2M)-1-ethyl-3-(3-hydroxy-2,2-dimethylpropyl)-2-{2-[(1S)-1-methoxyethyl]pyridin-3-yl}indol-5-yl]-5-fluoro-5,6-dihydro-2H-pyridin-1-yl}propanoyl]-1,2-diazinane-3-carboxylic acid as an oil. ESI-MS m/z=765.3 [M+H]⁺; Calculated MW: 764.4.
Step 8. Into a 250 mL round-bottom flask were added (3S)-1-[(2S)-2-[(tert-butoxycarbonyl)amino]-3-{3-[(2M)-1-ethyl-3-(3-hydroxy-2,2-dimethylpropyl)-2-{2-[(1S)-1-methoxyethyl]pyridin-3-yl}indol-5-yl]-5-fluoro-5,6-dihydro-2H-pyridin-1-yl}propanoyl]-1,2-diazinane-3-carboxylic acid (340 mg, 0.45 mmol, 1 equiv), DCM (100 mL), DIEA (1723 mg, 13.3 mmol, 30 equiv), HOBT (600.60 mg, 4.440 mmol, 10 equiv) and EDCl (2556.23 mg, 13.320 mmol, 30 equiv) at 0° C. The resulting mixture was stirred for 6 h at room temperature under air atmosphere. The reaction was quenched with water at 0° C. The resulting mixture was extracted with CH₂Cl₂ (3×30 mL). The combined organic layers were dried over anhydrous Na₂SO₄. After filtration, the filtrate was concentrated under reduced pressure. The residue was purified by silica gel column chromatography) to afford tert-butyl ((6³S,4S)-1¹-ethyl-23-fluoro-1²-(2-((S)-1-methoxyethyl)pyridin-3-yl)-10,10-dimethyl-5,7-dioxo-2¹,2²,2³,2⁶,6¹,6²,6³,6⁴,6⁵,6⁶-decahydro-1¹H-8-oxa-1(5,3)-indola-6(1,3)-pyridazina- 2(5,1)-pyridinacycloundecaphane-4-yl)carbamate (220 mg, 66% yield) as a yellow solid. ESI-MS m/z=747.5 [M+H]⁺; Calculated MW: 746.4.
Step 9. Into a 40 mL vial were added tert-butyl ((6³S,4S)-1¹-ethyl-23-fluoro-1²-(2-((S)-1-methoxyethyl)pyridin-3-yl)-10,10-dimethyl-5,7-dioxo-2¹,2²,2³,2⁶,6¹,6²,6³,6⁴,6⁵,6⁶-decahydro-1¹H-8-oxa-1(5,3)-indola-6(1,3)-pyridazina-2(5, 1)-pyridinacycloundecaphane-4-yl)carbamate (220 mg, 0.295 mmol, 1 equiv), DCM (3 mL) and TFA (1 mL, 26.926 mmol, 91.42 equiv) at 0° C. The resulting mixture was stirred for 3 h at room temperature under air atmosphere. The reaction was quenched with sat. NaHCO₃(aq.) at 0° C. The resulting mixture was extracted with CH₂Cl₂ (3×10 mL). The combined organic layers were dried over anhydrous Na_{2S}O₄. After filtration, the filtrate was concentrated under reduced pressure to afford (6³S,4S)-4-amino-1¹-ethyl-2³-fluoro-1²-(2-((S)-1-methoxyethyl)pyridin-3-yl)-10,10-dimethyl-2¹,2²,2³,2⁶,6¹,6²,6³,6⁴,6⁵,6⁶- decahydro-1¹H-8-oxa-1(5,3)-indola-6(1,3)-pyridazina-2(5,1)-pyridinacycloundecaphane-5,7-dione (210 mg, 88% yield) as a white solid. ESI-MS m/z=647.3 [M+H]⁺; Calculated MW: 646.4.
Step 10. Into a 40 mL vial were added (6³S,4S)-4-amino-1¹-ethyl-23-fluoro-1²-(2-((S)-1-methoxyethyl)pyridin-3-yl)-10,10-dimethyl-2¹,2²,2³,2⁶,6¹,6²,6³,6⁴,6⁵,6⁶-decahydro-1¹H-8-oxa-1(5,3)-indola-6(1,3)-pyridazina-2(5, 1)-pyridinacycloundecaphane-5,7-dione (200 mg, 0.31 mmol, 1 equiv), DCM (2 mL), DIEA (239.8 mg, 1.9 mmol, 6 equiv), (2S)-3-methyl-2-[methyl (4-(prop-2-enoyl)-1-oxa-4,9-diazaspiro[5.5]undecane-9-carbonyl)amino]butanoic acid (136.3 mg, 0.37 mmol, 1.2 equiv) and HATU (176.4 mg, 0.46 mmol, 1.5 equiv) at 0° C. The resulting mixture was stirred for 2 h at 0° C. under air atmosphere. The reaction was quenched with water at 0° C. The resulting mixture was extracted with CH₂Cl₂ (3×20 mL). The combined organic layers were dried over anhydrous Na_{2S}SO₄. After filtration, the filtrate was concentrated under reduced pressure. The crude product (200 mg) was purified by Prep-HPLC to afford 4-acryloyl-N-((2S)-1-(((6³S,4S)-1¹-ethyl-24-fluoro-1²-(2-((S)-1-methoxyethyl)pyridin-3-yl)-10,10-dimethyl-5,7-dioxo-2¹,2²,2³,2⁶,6¹,6²,6³,6⁴,6⁵,6⁶-decahydro-1¹H-8-oxa-1(5,3)-indola-6(1,3)-pyridazina-2(5,1)- pyridinacycloundecaphane-4-yl)amino)-3-methyl-1-oxobutan-2-yl)-N-methyl-1-oxa-4,9-diazaspiro[5.5]undecane-9-carboxamide (61.7 mg, 20% yield) as a white solid. ESI-MS m/z=996.7 [M+H]⁺; Calculated MW: 995.6. ¹H NMR (400 MHZ, DMSO-d₆) δ 8.77 (dd, J=4.7, 1.7 Hz, 1H), 7.99-7.81 (m, 2H), 7.61-7.45 (m, 3H), 7.40 (s, 1H), 6.83 (ddd, J=16.1, 10.3, 5.2 Hz, 1H), 6.16 (dd, J=16.2, 6.6 Hz, 1H), 5.77-5.48 (m, 3H), 4.31 (d, J=12.5 Hz, 1H), 4.13 (dq, J=38.6, 7.1, 6.6 Hz, 2H), 3.93-3.67 (m, 4H), 3.69-3.52 (m, 6H), 3.51 (s, 2H), 3.32-3.05 (m, 4H), 2.93 (s, 5H), 2.84-2.60 (m, 7H), 2.57 (d, J=10.9 Hz, 1H), 2.39 (s, 2H), 2.24 (s, 1H), 2.15-1.91 (m, 2H), 1.81 (d, J=12.0 Hz, 1H), 1.75-1.47 (m, 5H), 1.42 (d, J=6.1 Hz, 4H), 1.10 (t, J=7.1 Hz, 3H), 0.81 (dd, J=23.8, 7.0 Hz, 9H), 0.55 (s, 3H).

### Example 16. Synthesis of (4S)-3-acryloyl-N-((2S)-1-(((6³S,4S)-1²-(2-((S)-1-methoxyethyl)pyridin-3-yl)-10,10-dimethyl-5,7-dioxo-1¹-(2,2,2-trifluoroethyl)-2¹,2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-4-yl)amino)-3-methyl-1-oxobutan-2-yl)-N,4-dimethyl-1-oxa-3,8-diazaspiro[4.5]decane-8-carboxamide (A221)

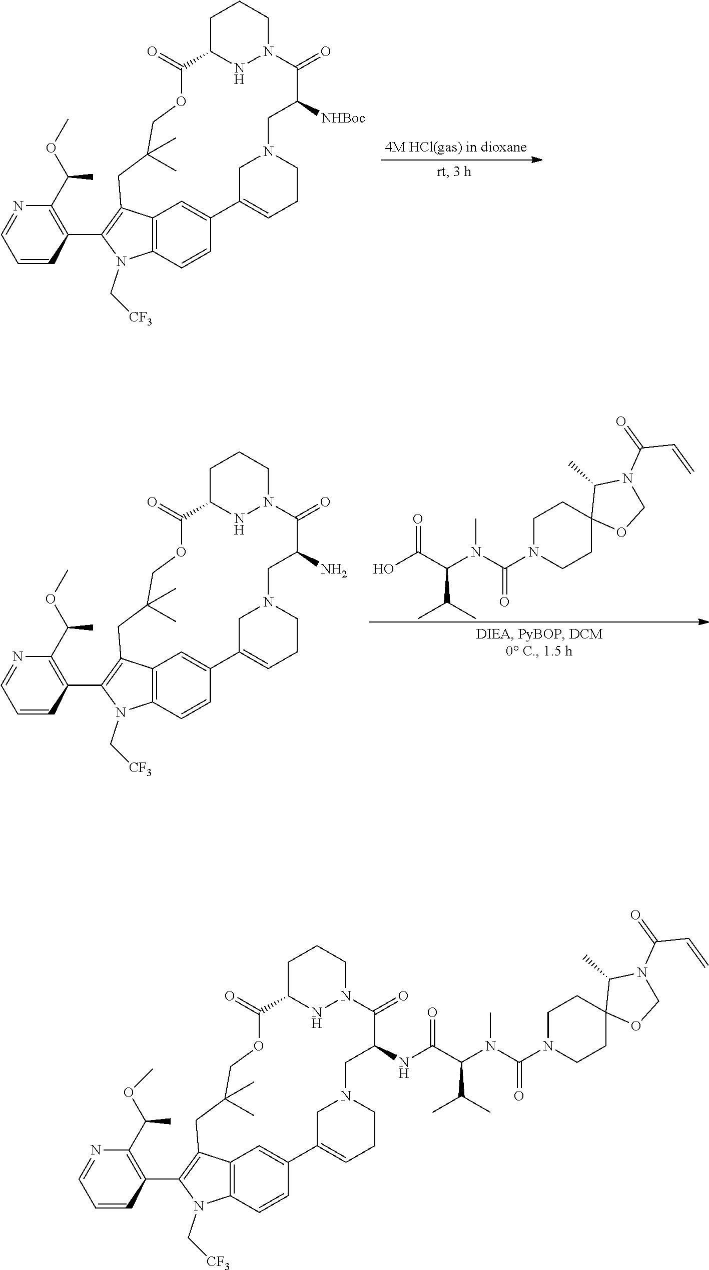

Step 1. Into a 2 L vial were added tert-butyl ((6³S,4S)-1²-(2-((S)-1-methoxyethyl)pyridin-3-yl)-10,10-dimethyl-5,7-dioxo-1¹-(2,2,2-trifluoroethyl)-2¹,2²,2³,2⁶,6¹,6²,6³,6⁴,6⁵,6⁶-decahydro-1¹H-8-oxa-1(5,3)-indola-6(1,3)-pyridazina-2(5, 1)-pyridinacycloundecaphane-4-yl)carbamate (70 g, 89.5 mmol, 1 equiv) in 1,4-dioxane (700 mL) was added 4N HCl in 1,4-dioxane (700 mL) at room temperature. The resulting mixture was stirred for 3 h at 25° C. under nitrogen atmosphere. The resulting mixture was concentrated under reduced pressure and the remaining residue was basified to pH=8 with saturated NaHCO₃ (2.0 L). The resulting mixture was extracted with EtOAc (3×700 mL). The combined organic layers were washed with brine (2×100 mL) and dried over anhydrous Na₂SO₄. After filtration, the filtrate was concentrated under reduced pressure to afford crude (6³S,4S)-4-amino-1²-(2-((S)-1-methoxyethyl)pyridin-3-yl)-10,10-dimethyl-1¹-(2,2,2-trifluoroethyl)-2¹,2²,2³,2⁶,6¹,6²,6³,6⁴,6⁵,6⁶-decahydro-1¹H-8-oxa-1(5,3)-indola-6(1,3)-pyridazina-2(5,1)- pyridinacycloundecaphane-5,7-dione (61 g) as an off-white solid (A1). ESI-MS m/z=683.3 [M+H]⁺. Calculated MW: 682.3.
Step 2. To a stirred mixture of (6³S,4S)-4-amino-1²-(2-((S)-1-methoxyethyl)pyridin-3-yl)-10,10-dimethyl-1¹-(2,2,2-trifluoroethyl)-2¹,2²,2³,2⁶,6¹,6²,6³,6⁴,6⁵,6⁶-decahydro-1¹H-8-oxa-1(5,3)-indola-6(1,3)- pyridazina-2(5,1)-pyridinacycloundecaphane-5,7-dione (61 g, 89.4 mmol, 1 equiv) in DCM (600 mL) was added DIEA (461.2 g, 3574.8 mmol, 40 equiv), N—((S)-3-acryloyl-4-methyl-1-oxa-3,8-diazaspiro[4.5]decane-8-carbonyl)-N-methyl-L-valine (39.39 g, 107.276 mmol, 1.2 equiv) (410 mL, 0.96 g/mL in DCM), PyBOP (13.98 g, 102.8 mmol, 1.15 equiv) in DCM (60 mL) dropwise at 0° C. under argon atmosphere. The resulting mixture was stirred for 2 h at 0° C. under nitrogen atmosphere. The mixture was then diluted with CH₂Cl₂ (600 mL). The combined organic layers were washed with brine (3×300 mL), dried over anhydrous Na₂SO₄. After filtration, the filtrate was concentrated under reduced pressure. The mixture was purified by Prep-HPLC to afford (4S)-3-acryloyl-N-((2S)-1-(((6³S,4S)-1²-(2-((S)-1-methoxyethyl)pyridin-3-yl)-10,10-dimethyl-5,7-dioxo-1¹-(2,2,2-trifluoroethyl)-2¹,2²,2³,2⁶,6¹,6²,6³,6⁴,6⁵,6⁶-decahydro-1¹H-8-oxa-1(5,3)-indola-6(1,3)- pyridazina-2(5,1)-pyridinacycloundecaphane-4-yl)amino)-3-methyl-1-oxobutan-2-yl)-N,4-dimethyl-1-oxa-3,8-diazaspiro[4.5]decane-8-carboxamide (30.2 g, 32.7% yield). ESI-MS m/z=1032.5 [M+H]⁺. Calculated MW: 1031.5. ¹H NMR (400 MHZ, DMSO-d₆) δ 8.77 (dd, J=4.7, 1.7 Hz, 1H), 7.94 (t, J=8.1 Hz, 1H), 7.79 (d, J=7.7 Hz, 1H), 7.65 (d, J=8.8 Hz, 1H), 7.60-7.50 (m, 2H), 7.44 (s, 1H), 6.61-6.15 (m, 3H), 5.74 (ddd, J=16.5, 8.9, 2.3 Hz, 2H), 5.48-5.33 (m, 2H), 5.20-5.05 (m, 1H), 5.02-4.92 (m, 1H), 4.59 (s, 1H), 4.27 (d, J=12.3 Hz, 1H), 4.07-3.83 (m, 5H), 3.69-3.57 (m, 2H), 3.50-3.42 (m, 1H), 3.32-3.30 (m, 1H), 3.28-3.14 (m, 2H), 3.04 (s, 4H), 2.87-2.66 (m, 7H), 2.57 (q, J=5.4 Hz, 1H), 2.48-2.43 (m, 1H), 2.23 (s, 2H), 2.08 (ddd, J=14.7, 12.1, 6.5 Hz, 1H), 1.98 (d, J=10.9 Hz, 1H), 1.81 (d, J=13.9 Hz, 1H), 1.75-1.48 (m, 6H), 1.38 (d, J=6.2 Hz, 3H), 1.10 (d, J=6.4 Hz, 3H), δ 0.85 (d, J=6.4 Hz, 3H), 0.80 (d, J=6.6 Hz, 3H), 0.71 (s, 3H), 0.59 (s, 3H).

### Example 17. Synthesis of (5R)-4-acryloyl-N-((2S)-1-(((6³S,4S)-1¹-ethyl-1²-(2-((S)-1-methoxyethyl)pyridin-3-yl)-10,10-dimethyl-5,7-dioxo-2¹,2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-4-yl)amino)-3-methyl-1-oxobutan-2-yl)-N,5-dimethyl-1-oxa-4,9-diazaspiro[5.5]undecane-9-carboxamide (A327)

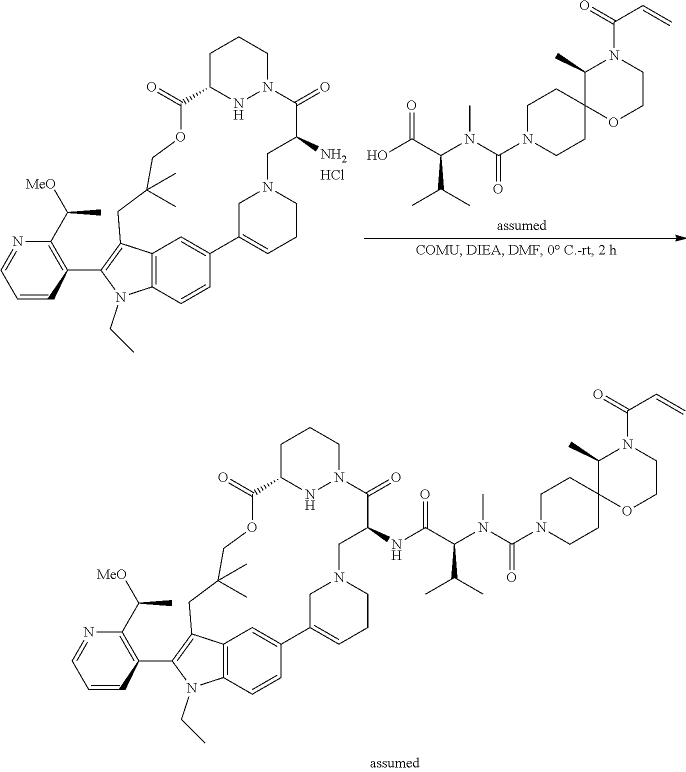

Step 1. Into a 40 mL vial were added (6³S,4S)-4-amino-1¹-ethyl-1²-(2-((S)-1-methoxyethyl)pyridin-3-yl)-10,10-dimethyl-2¹,2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-5,7-dione hydrochloride (85 mg, 0.13 mmol, 1 equiv), DMF (1 mL), N—((R)-4-acryloyl-5-methyl-1-oxa-4,9-diazaspiro[5.5]undecane-9-carbonyl)-N-methyl-L-valine (70 mg, 0.18 mmol, 1.4 equiv) and DIEA (670 mg, 5.2 mmol, 40.6 equiv) at 0° C. To a stirred solution was added COMU (50 mg, 0.1 mmol, 0.9 equiv) in DMF (0.5 mL) dropwise at 0° C. under argon atmosphere. The resulting mixture was stirred for 2 hours at room temperature under argon atmosphere. The reaction was quenched with water/ice and the resulting mixture was extracted with EtOAc (3×20 mL). The combined organic layers were washed with saturated sodium chloride solution (3×10 mL), dried over anhydrous Na₂SO₄, and filtered. The filtrate was concentrated under reduced pressure and the remaining residue was purified by Prep-HPLC to afford (5R)-4-acryloyl-N-((2S)-1-(((6³S,4S)-1¹-ethyl-1²-(2-((S)-1-methoxyethyl)pyridin-3-yl)-10,10-dimethyl-5,7-dioxo-2¹,2²,2³,2⁶,6¹,6²,6³,6⁴,6⁵,6⁶-decahydro-1¹H-8-oxa-1(5,3)-indola-6(1,3)-pyridazina-2(5,1)- pyridinacycloundecaphane-4-yl)amino)-3-methyl-1-oxobutan-2-yl)-N,5-dimethyl-1-oxa-4,9-diazaspiro[5.5]undecane-9-carboxamide (4.4 mg, 3.5) as a white solid. ESI-MS m/z=992.7

[M+H]⁺; Calculated MW: 991.6. ¹H NMR (400 MHZ, DMSO-d₆) δ 8.69 (dd, J=4.7, 1.8 Hz, 1H), 7.84 (dd, J=30.9, 8.4 Hz, 2H), 7.50-7.38 (m, 3H), 7.35 (s, 1H), 6.73 (dd, J=16.6, 10.5 Hz, 1H), 6.23 (s, 1H), 6.08 (d, J=16.6 Hz, 1H), 5.75-5.59 (m, 2H), 5.54 (d, J=12.1 Hz, 1H), 4.39-4.17 (m, 2H), 4.15-4.04 (m, 2H), 3.98 (d, J=9.1 Hz, 2H), 3.85-3.63 (m, 4H), 3.57 (d, J=11.5 Hz, 4H), 3.14 (s, 1H), 2.88 (d, J=31.1 Hz, 8H), 2.71 (t, J=12.4 Hz, 1H), 2.57 (d, J=18.7 Hz, 6H), 2.38-1.94 (m, 7H), 1.88 (d, J=11.4 Hz, 1H), 1.74 (d, J=12.3 Hz, 1H), 1.67-1.41 (m, 3H), 1.34 (d, J=6.1 Hz, 4H), 1.27-0.93 (m, 9H), 0.90-0.58 (m, 10H), 0.45 (s, 3H).

### Example 18. Synthesis of (5S)-4-acryloyl-N-((2S)-1-(((6³S,4S)-1¹-ethyl-1²-(2-((S)-1-methoxyethyl)pyridin-3-yl)-10,10-dimethyl-5,7-dioxo-2¹,2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-4-yl)amino)-3-methyl-1-oxobutan-2-yl)-N,5-dimethyl-1-oxa-4,9-diazaspiro[5.5]undecane-9-carboxamide (A328)

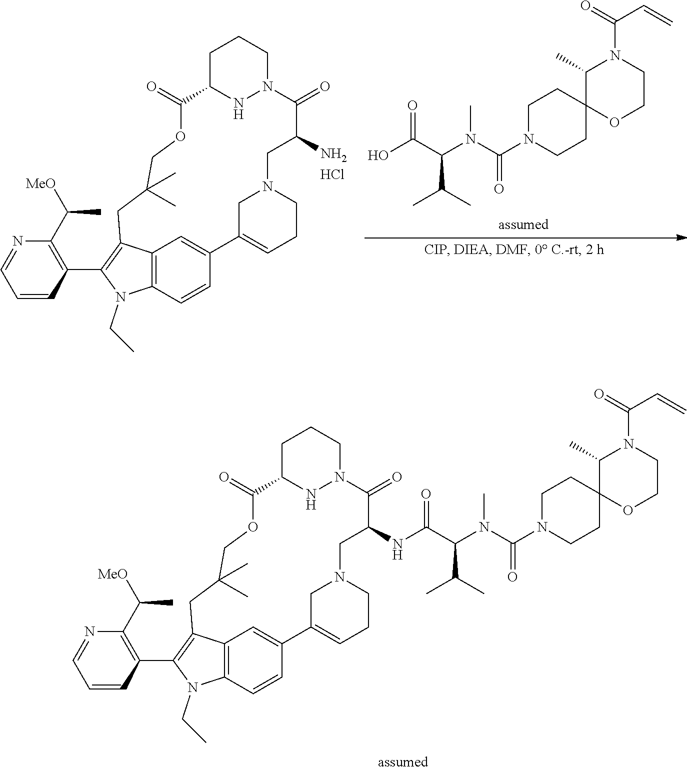

Step 1. Into a 40 mL vial were added (6³S,4S)-4-amino-1¹-ethyl-1²-(2-((S)-1-methoxyethyl)pyridin-3-yl)-10,10-dimethyl-2¹,2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-5,7-dione hydrochloride (100 mg, 0.15 mmol, 1 equiv), DMF (1 mL), DIEA (800 mg, 6.2 mmol, 41.2 equiv) and (N—(S)-4-acryloyl-5-methyl-1-oxa-4,9-diazaspiro[5.5]undecane-9-carbonyl)-N-methyl-L-valine (80 mg, 0.21 mmol, 1.4 equiv) at 0° C. CIP (45 mg, 0.16 mmol, 1.1 equiv) in DMF (0.5 mL) was then added dropwise at 0° C. under argon atmosphere. The resulting mixture was stirred for 2 hours at room temperature under argon atmosphere, quenched with water/ice and the resulting mixture was extracted with EtOAc (3×20 mL). The combined organic layers were washed with saturated sodium chloride solution (3×10 mL), dried over anhydrous Na₂SO₄, and filtered. The filtrate was concentrated under reduced pressure and the resulting residue was purified by Prep-HPLC to afford (5S)-4-acryloyl-N-((2S)-1-(((6³S,4S)-1¹-ethyl-1²-(2-((S)-1-methoxyethyl)pyridin-3-yl)-10,10-dimethyl-5,7-dioxo-2¹,2²,2³,2⁶,6¹,6²,6³,6⁴,6⁵,6⁶-decahydro-1¹H- 8-oxa-1(5,3)-indola-6(1,3)-pyridazina-2(5, 1)-pyridinacycloundecaphane-4-yl)amino)-3-methyl-1-oxobutan-2-yl)-N,5-dimethyl-1-oxa-4,9-diazaspiro[5.5]undecane-9-carboxamide (6.6 mg, 4.4%) as a white solid. ESI-MS m/z=992.7 [M+H]⁺; Calculated MW: 991.6. ¹H NMR (400 MHZ, DMSO-d₆) δ 8.69 (d, J=4.6 Hz, 1H), 8.04-7.69 (m, 2H), 7.60-7.26 (m, 4H), 6.74 (t, J=13.7 Hz, 1H), 6.24 (s, 1H), 6.08 (d, J=17.0 Hz, 1H), 5.81-5.40 (m, 3H), 4.43-4.18 (m, 2H), 4.16-3.86 (m, 4H), 3.74 (td, J=28.3, 24.5, 9.6 Hz, 4H), 3.55 (d, J=4.6 Hz, 4H), 3.04 (s, 4H), 3.00-2.66 (m, 8H), 2.57 (d, J=17.7 Hz, 6H), 2.38-2.08 (m, 4H), 1.94 (dd, J=41.0, 10.4 Hz, 3H), 1.74 (d, J=11.8 Hz, 1H), 1.65-1.25 (m, 8H), 1.23-0.92 (m, 7H), 0.90-0.59 (m, 10H), 0.46 (s, 3H).

### Example 19. Synthesis of 4-acryloyl-N-((2S)-1-(((6³S,4S)-1¹-ethyl-10,10-difluoro-1²-(2-((S)-1-methoxyethyl)pyridin-3-yl)-5,7-dioxo-2¹,2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-4-yl)amino)-3-methyl-1-oxobutan-2-yl)-N-methyl-1-oxa-4,9-diazaspiro[5.5]undecane-9-carboxamide (A250)

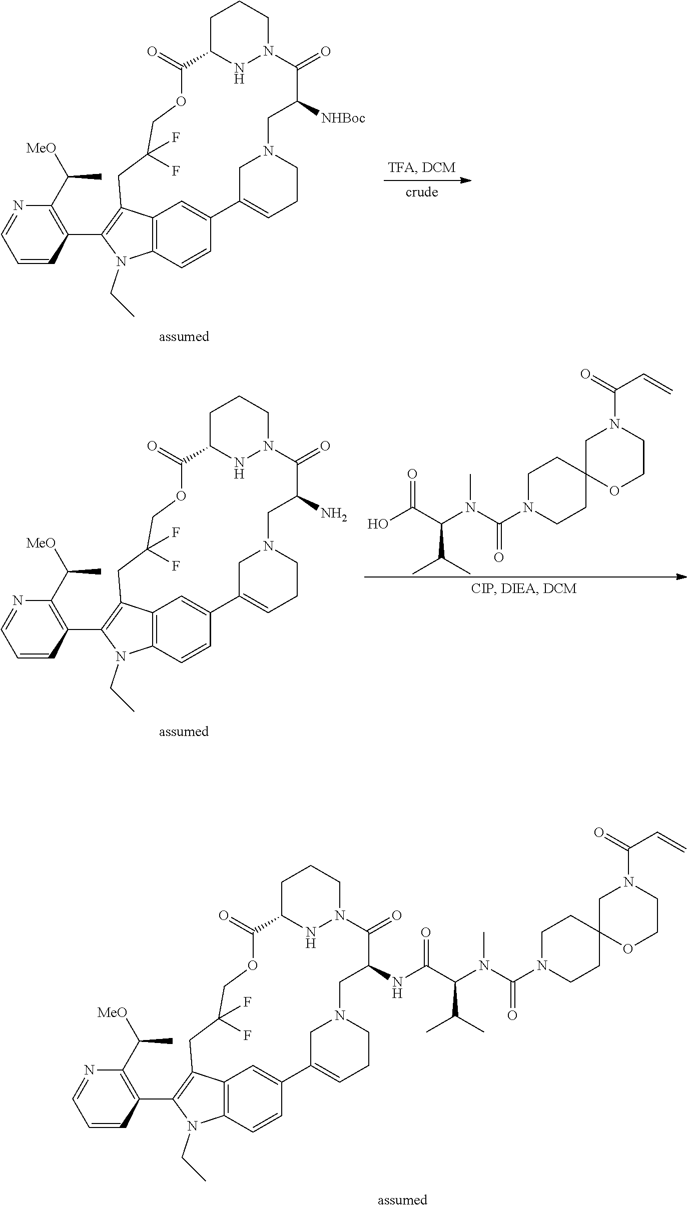

Step 1. A mixture of tert-butyl ((6³S,4S)-1¹-ethyl-10,10-difluoro-1²-(2-((S)-1-methoxyethyl)pyridin-3-yl)-5,7-dioxo-2¹,2²,2³,2⁶,6¹,6²,6³,6⁴,6⁵,6⁶-decahydro-1¹H-8-oxa-1(5,3)-indola-6(1,3)-pyridazina-2(5,1)- pyridinacycloundecaphane-4-yl)carbamate (80 mg, 0.12 mmol, 1.0 equiv) and TFA (0.3 mL, 4.0 mmol, 32.1 equiv) in DCM (1 mL) was stirred for 2 h at 0° C. under nitrogen atmosphere. The mixture was acidified to pH 9 with NaHCO₃ and extracted with CH₂Cl₂ (3×10 mL). The combined organic layers were washed with brine (3×10 mL), dried over anhydrous Na₂SO₄, and filtered. The filtrate was concentrated under reduced pressure to afford (6³S,4S)-4-amino-1¹-ethyl-10,10-difluoro-1²-(2-((S)-1-methoxyethyl)pyridin-3-yl)-2¹,2²,2³,2⁶,6¹,6²,6³,6⁴,6⁵,6⁶-decahydro-1¹H-8-oxa-1(5,3)-indola-6(1,3)-pyridazina-2(5,1)- pyridinacycloundecaphane-5,7-dione (70 mg, crude) as a light yellow oil. ESI-MS m/z=637.3 [M+H]⁺; Calculated MW: 636.3.
Step 2. A solution of (63 S,4S)-4-amino-1¹-ethyl-10,10-difluoro-1²-(2-((S)-1-methoxyethyl)pyridin-3-yl)-2¹,2²,2³,2⁶,6¹,6²,6³,6⁴,6⁵,6⁶-decahydro-1¹H-8-oxa-1(5,3)-indola-6(1,3)-pyridazina-2(5,1)- pyridinacycloundecaphane-5,7-dione (60 mg, 0.09 mmol, 1.0 equiv) in DCM (1 mL) was treated with DIEA (487 mg, 3.7 mmol, 40 equiv) for 1 min at 0° C. under nitrogen atmosphere followed by the addition of (2S)-3-methyl-2-[methyl (4-(prop-2-enoyl)-1-oxa-4,9-diazaspiro[5.5]undecane-9-carbonyl)amino]butanoic acid (41 mg, 0.1 mmol, 1.2 equiv) and CIP (31.5 mg, 0.1 mmol, 1.2 equiv) in portions at room temperature. The resulting mixture was stirred for 2 h at room temperature under nitrogen atmosphere. The resulting mixture was extracted with CH₂Cl₂ (3×10 mL), and the combined organic layers were washed with brine (3×10 mL), dried over anhydrous Na₂SO₄, and filtered. The filtrate was concentrated under reduced pressure and the crude product (70 mg) was purified by Prep-HPLC to afford 4-acryloyl-N-((2S)-1-(((6³S,4S)-1¹-ethyl-10,10-difluoro-1²-(2-((S)-1-methoxyethyl)pyridin-3-yl)-5,7-dioxo-2¹,2²,2³,2⁶,6¹,6²,6³,6⁴,6⁵,6⁶-decahydro-1¹H-8-oxa-1(5,3)-indola-6(1,3)-pyridazina-2(5, 1)-pyridinacycloundecaphane-4-yl)amino)-3-methyl-1-oxobutan-2-yl)-N-methyl-1-oxa-4,9-diazaspiro[5.5]undecane-9-carboxamide (7.2 mg, 7% yield) as an off-white solid ESI-MS m/z=986.7 [M+H]⁺; Calculated MW: 985.5. ¹H NMR (400 MHZ, DMSO-d₆) δ 8.77-8.71 (m, 1H), 7.89 (s, 1H), 7.80 (d, J=7.6 Hz, 1H), 7.50 (ddd, J=18.8, 14.5, 8.5 Hz, 4H), 6.96-6.72 (m, 1H), 6.17 (d, J=21.3 Hz, 2H), 5.79-5.68 (m, 2H), 5.39 (d, J=12.0 Hz, 1H), 4.34-4.13 (m, 2H), 4.12-3.95 (m, 4H), 3.94-3.77 (m, 2H), 3.62 (d, J=23.2 Hz, 4H), 3.47 (dd, J=22.2, 19.3 Hz, 4H), 3.28-3.13 (m, 3H), 3.10-3.06 (m, 6H), 2.98 (s, 1H), 2.76 (s, 3H), 2.70 (s, 3H), 2.21 (s, 2H), 2.11-2.04 (m, 1H), 1.97 (d, J=10.6 Hz, 1H), 1.80 (s, 1H), 1.64-1.53 (m, 5H), 1.42 (s, 1H), 1.33 (d, J=6.2 Hz, 3H), 0.99 (t, J=7.1 Hz, 3H), 0.81 (dd, J=18.0, 6.6 Hz, 6H).

### Example 20. Synthesis of (2R,4R)-3-acryloyl-N-((2S)-1-(((22S,63S,4$)-12-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)amino)-3-methyl-1-oxobutan-2-yl)-N,2,4-trimethyl-1-oxa-3,8-diazaspiro[4.5]decane-8-carboxamide (A294)

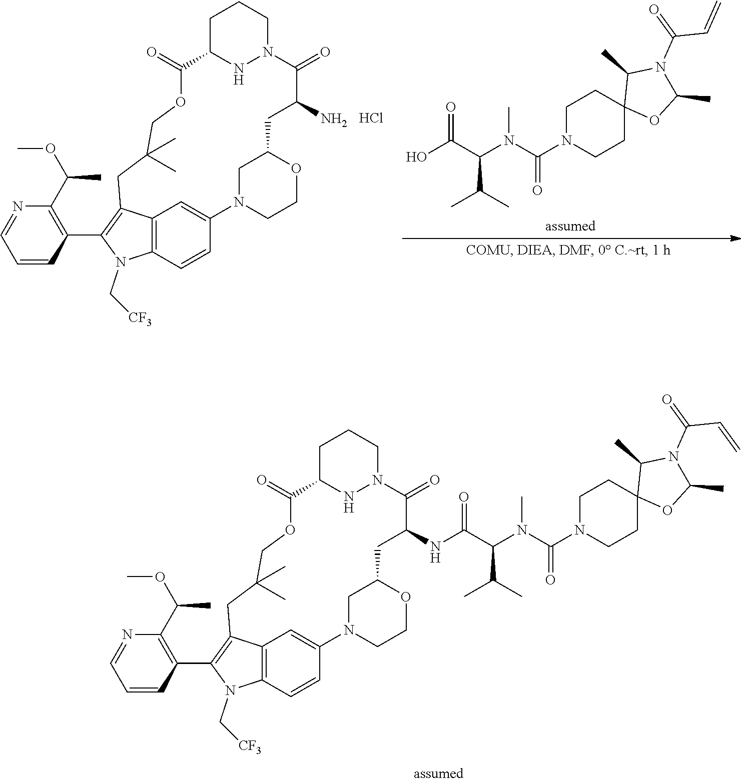

Step 1. Into a 40 mL vial were added (2²S,6³S,4S)-4-amino-1²-(2-((S)-1-methoxyethyl)pyridin-3-yl)-10,10-dimethyl-1¹-(2,2,2-trifluoroethyl)-6¹,6²,6³,6⁴,6⁵,6⁶-hexahydro-1¹H-8-oxa-2(4,2)-morpholina-1(5,3)-indola-6(1,3)-pyridazinacycloundecaphane-5,7-dione hydrochloride (150 mg, 0.22 mmol, 1.0 equiv), N-((2R,4R)-3-acryloyl-2,4-dimethyl-1-oxa-3,8-diazaspiro[4.5]decane-8-carbonyl)-N-methyl-L-valine (124.9 mg, 0.32 mmol, 1.5 equiv), DMF, DIEA (1129 mg, 8.7 mmol, 40 equiv), COMU (112 mg, 0.26 mmol, 1.2 equiv) at room temperature. The resulting mixture was stirred for 1 hour at 0° C. under air atmosphere. The reaction was then quenched with water/ice at 0° C. The resulting mixture was extracted with EtOAc (4×30 mL), and the combined organic layers were dried over anhydrous Na₂SO₄ and filtered. The filtrate was concentrated under reduced pressure and the resulting residue was purified by Prep-HPLC to afford (2R,4R)-3-acryloyl-N-((2S)-1-(((2²S,6³S,4^{S})-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)amino)-3-methyl-1-oxobutan-2-yl)-N,2,4-trimethyl-1-oxa-3,8-diazaspiro[4.5]decane-8-carboxamide (92 mg, 40%) as a white solid. ESI-MS m/z=1051.5 [M+H]⁺; Calculated MW: 1049.6. ¹H NMR (400 MHZ, DMSO-d₆) δ 8.75 (dd, J=4.7, 1.7 Hz, 1H), 7.75 (d, J=7.6 Hz, 2H), 7.64-7.45 (m, 2H), 7.08 (d, J=9.2 Hz, 1H), 6.96 (d, J=2.1 Hz, 1H), 6.67-6.47 (m, 1H), 6.22 (d, J=16.6 Hz, 1H), 5.75 (d, J=10.4 Hz, 1H), 5.42 (d, J=31.1 Hz, 2H), 5.29-5.05 (m, 2H), 4.68 (s, 1H), 4.26 (d, J=12.0 Hz, 1H), 4.15-3.98 (m, 2H), 3.79 (t, J=12.6 Hz, 3H), 3.57 (m, 5H), 3.25 (s, 2H), 3.16 (s, 4H), 3.04 (s, 1H), 2.74 (s, 5H), 2.17-1.96 (m, 3H), 1.95-1.46 (m, 11H), 1.38 (dd, J=18.1, 5.6 Hz, 7H), 1.14 (d, J=6.5 Hz, 3H), 0.92-0.69 (m, 10H), 0.37 (s, 3H).

### Example 21. Synthesis of (2S,4R)-3-acryloyl-N-((2S)-1-(((2²S,6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)amino)-3-methyl-1-oxobutan-2-yl)-N,2,4-trimethyl-1-oxa-3,8-diazaspiro[4.5]decane-8-carboxamide (A309)

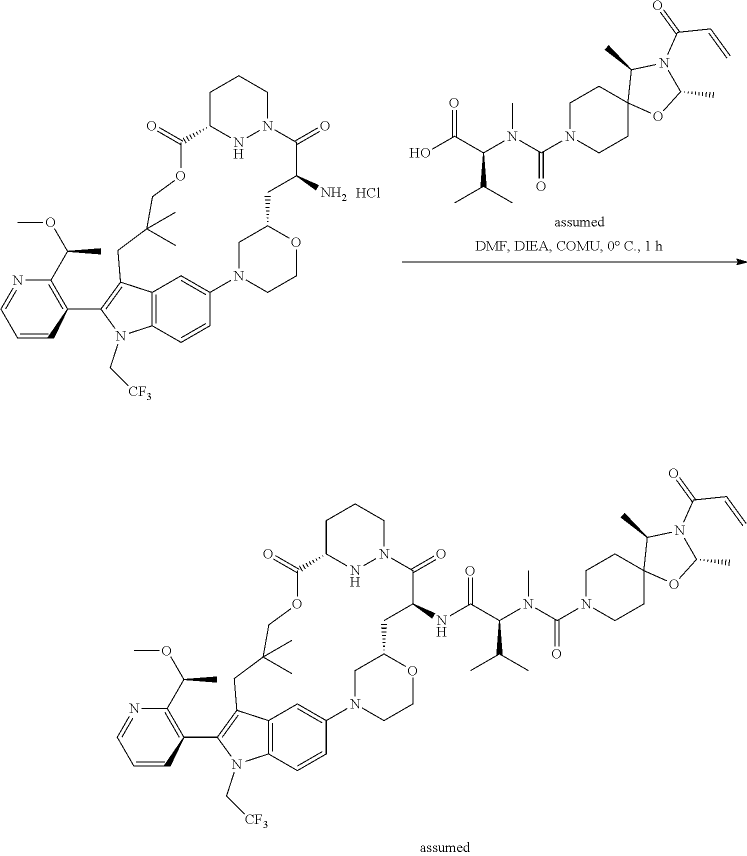

Step 1. Into a 8 mL vial were added (2²S,6³S,4S)-4-amino-1²-(2-((S)-1-methoxyethyl)pyridin-3-yl)-10,10-dimethyl-1¹-(2,2,2-trifluoroethyl)-6¹,6²,6³,6⁴,6⁵,6⁶-hexahydro-1¹H-8-oxa-2(4,2)-morpholina-1(5,3)-indola-6(1,3)-pyridazinacycloundecaphane-5,7-dione hydrochloride (36 mg, 0.050 mmol, 1.00 equiv), N-((2S,4R)-2,4-dimethyl-3-(prop-1-en-2-yl)-1-oxa-3,8-diazaspiro[4.5]decane-8-carbonyl)-N-methyl-L-valine (22.7 mg, 0.06 mmol, 1.2 equiv), DMF (0.5 mL), DIEA (257 mg, 2.0 mmol, 40 equiv) and COMU (21.30 mg, 0.050 mmol, 1 equiv) at 0° C. The resulting mixture was stirred for 1 hour at 0° C. The reaction was quenched with water/ice at 0° C. and extracted with EtOAc (3×20 mL). The combined organic layers were washed with brine (3×10 mL), dried over anhydrous Na₂SO₄, and filtered. The filtrate was concentrated under reduced pressure and the remaining residue was purified by Prep-HPLC to afford (2S,4R)-3-acryloyl-N-((2S)-1-(((22S,63 S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)amino)-3-methyl-1-oxobutan-2-yl)-N,2,4-trimethyl-1-oxa-3,8-diazaspiro[4.5]decane-8-carboxamide (7 mg, 13.4%) as a white solid. ESI-MS m/z=1050.8 [M+H]⁺; Calculated MW: 1049.6. ¹H NMR (400 MHZ, DMSO-d₆) δ 8.75 (d, J=4.4 Hz, 1H), 7.76 (d, J=8.0 Hz, 2H), 7.64-7.46 (m, 2H), 7.04 (d, J=53.9 Hz, 2H), 6.67 (dd, J=16.5, 10.4 Hz, 1H), 6.21 (d, J=16.4 Hz, 1H), 5.74 (d, J=10.1 Hz, 1H), 5.53-5.28 (m, 3H), 5.15 (s, 1H), 4.68 (s, 1H), 4.26 (d, J=16.3 Hz, 2H), 4.11 (d, J=6.3 Hz, 1H), 3.78 (d, J=11.1 Hz, 3H), 3.59 (s, 7H), 3.17 (s, 8H), 2.72 (s, 6H), 2.03 (d, J=34.4 Hz, 2H), 1.78 (d, J=14.7 Hz, 5H), 1.69-1.44 (m, 7H), 1.36 (d, J=6.1 Hz, 4H), 1.26-1.01 (m, 4H), 0.91-0.71 (m, 10H), 0.36 (s, 3H).

TABLE 3
Exemplary Compounds Prepared by
Methods of the Present Invention

| | | Observed MW |
|---|---|---|
| | Calc'd MW | LCMS (ESI) |
| Ex # | [M + H] | m/z [M + H] |
| A1 | 937.49 | 938.7 |
| A2 | 1079.58 | 1081.1 |
| A3 | 1077.58 | 1078.7 |
| A4 | 1070.63 | 1071.5 |
| A5 | 1108.61 | 1109.8 |
| A6 | 1086.64 | 1087.9 |
| A7 | 1030.49 | 1031.9 |
| A8 | 1054.64 | 1055.7 |
| A9 | 1039.55 | 1040.9 |
| A10 | 965.48 | 966.2 |
| A11 | 999.47 | 1000.7 |
| A12 | 1005.53 | 1006.9 |
| A13 | 990.54 | 991.9 |
| A14 | 1086.64 | 1087.8 |
| A15 | 1072.63 | 1073.4 |
| A16 | 953.46 | 954.4 |
| A17 | 1046.5 | 1047.6 |
| A18 | 1013.5 | 1014.8 |
| A19 | 958.53 | 959.6 |
| A20 | 967.55 | 968.3 |
| A21 | 939.52 | 940.5 |
| A22 | 1065.59 | 1066.8 |
| A23 | 989.46 | 990.5 |
| A24 | 1009.52 | 1010.8 |
| A25 | 999.48 | 1000.7 |
| A26 | 1005.53 | 1006.9 |
| A27 | 949.54 | 950.7 |
| A28 | 1032.56 | 1033.4 |
| A29 | 1017.49 | 1018.9 |
| A30 | 1099.59 | 1100.8 |
| A31 | 1015.59 | 1016.8 |
| A32 | 1068.58 | 1069.6 |
| A33 | 1017.49 | 1018.7 |
| A34 | 1063.61 | 1065.1 |
| A35 | 1013.61 | 1014.4 |
| A36 | 974.54 | 975.6 |
| A37 | 949.51 | 950.5 |
| A38 | 1008.62 | 1009.6 |
| A39 | 1064.6 | 1065.6 |
| A40 | 944.52 | 945.8 |
| A41 | 981.57 | 982.8 |
| A42 | 981.57 | 982.3 |
| A43 | 1091.65 | 1092.6 |
| A44 | 1065.64 | 1066.6 |
| A45 | 930.5 | 931.7 |
| A46 | 995.58 | 996.8 |
| A47 | 977.57 | 978.2 |
| A48 | 981.57 | 982.8 |
| A49 | 976.52 | 977.5 |
| A50 | 1082.63 | 1083.9 |
| A51 | 1030.64 | 1031.5 |
| A52 | 1061.64 | 1062.5 |
| A53 | 1096.65 | 1098 |
| A54 | 1018.64 | 1019.6 |
| A55 | 976.59 | 977.5 |
| A56 | 1018.64 | 1019.5 |
| A57 | 971.56 | 972.7 |
| A58 | 977.57 | 978.8 |
| A59 | 1048.57 | 1049.5 |
| A60 | 1110.66 | 1112.1 |
| A61 | 948.56 | 949.5 |
| A62 | 1096.65 | 1097.9 |
| A63 | 977.57 | 978.8 |
| A64 | 977.57 | 978.8 |
| A65 | 962.57 | 963.45 |
| A66 | 1005.53 | 1006.8 |
| A67 | 990.61 | 991.6 |
| A68 | 999.56 | 1000.6 |
| A69 | 1020.62 | 1021.5 |
| A70 | 962.57 | 963.4 |
| A71 | 951.56 | 952.6 |
| A72 | 1022.61 | 1023.6 |
| A73 | 994.6 | 995.5 |
| A74 | 1016.62 | 1017.6 |
| A75 | 1059.66 | 1060.6 |
| A76 | 1040.6 | 1041.6 |
| A77 | 1034.63 | 1035.5 |
| A78 | 949.51 | 950.7 |
| A79 | 1017.49 | 1018.7 |
| A80 | 1022.63 | 1023.6 |
| A81 | 1004.62 | 1005.5 |
| A82 | 1022.63 | 1023.6 |
| A83 | 1099.69 | 1100.6 |
| A84 | 1008.62 | 1009.5 |
| A85 | 1113.64 | 1114.6 |
| A86 | 1031.6 | 1032.9 |
| A87 | 1026.61 | 1027.5 |
| A88 | 991.59 | 992.8 |
| A89 | 1056.62 | 1057.8 |
| A90 | 1007.58 | 1008.8 |
| A91 | 944.52 | 945.9 |
| A92 | 1036.56 | 1037.9 |
| A93 | 977.57 | 978.8 |
| A94 | 1005.53 | 1006.8 |
| A95 | 1033.58 | 1034.9 |
| A96 | 1058.59 | 1059.6 |
| A97 | 1062.58 | 1063.9 |
| A98 | 1011.58 | 1012.8 |
| A99 | 1063.57 | 1064.8 |
| A100 | 1049.55 | 1050.8 |
| A101 | 1061.59 | 532.2 |
| A102 | 1063.57 | 1064.8 |
| A103 | 1033.56 | 1034.8 |
| A104 | 928.52 | 929.8 |
| A105 | 951.47 | 952.3 |
| A106 | 1001.56 | 1002.5 |
| A107 | 1028.51 | 1029.6 |
| A108 | 953.54 | 954.4 |
| A109 | 1003.51 | 1004.8 |
| A110 | 1061.61 | 1062.9 |
| A111 | 1029.61 | 1030.9 |
| A112 | 1103.69 | 1104.5 |
| A113 | 887.47 | 888.4 |
| A114 | 953.54 | 954.5 |
| A115 | 1080.6 | 1081.9 |
| A116 | 991.51 | 992.7 |
| A117 | 1060.52 | 1062.7 |
| A118 | 994.54 | 995.4 |
| A119 | 978.52 | 979.9 |
| A120 | 1079.6 | 1080.8 |
| A121 | 951.56 | 952.4 |
| A122 | 1064.6 | 1065.8 |
| A123 | 937.49 | 938.8 |
| A124 | 973.54 | 974.7 |
| A125 | 1095.66 | 1097 |
| A126 | 1089.67 | 1090.7 |
| A127 | 994.54 | 995.8 |
| A128 | 983.56 | 984.5 |
| A129 | 1044.6 | 1045.6 |
| A130 | 995.58 | 996.5 |
| A131 | 1020.56 | 1021.3 |
| A132 | 954.46 | 955.4 |
| A133 | 985.58 | 986.8 |
| A134 | 1084.57 | 1085.8 |
| A135 | 934.51 | 935.5 |
| A136 | 953.54 | 954.7 |
| A137 | 1100.55 | 1101.9 |
| A138 | 1047.6 | 1048.9 |
| A139 | 993.56 | 994.7 |
| A140 | 991.51 | 992.7 |
| A141 | 1027.57 | 1029.5 |
| A142 | 976.52 | 977.7 |
| A143 | 1057.54 | 1058.7 |
| A144 | 1046.58 | 1047.9 |
| A145 | 1025.61 | 1026.5 |
| A146 | 937.49 | 938.4 |
| A147 | 1009.6 | 1010.5 |
| A148 | 1059.56 | 1060.4 |
| A149 | 933.48 | 934.7 |
| A150 | 1093.59 | 1095 |
| A151 | 975.5 | 976.5 |
| A152 | 1046.58 | 1047.8 |
| A153 | 937.49 | 938.4 |
| A154 | 996.49 | 997.5 |
| A155 | 1067.6 | 1068.9 |
| A156 | 1062.59 | 1063.5 |
| A157 | 1147.57 | 1148.5 |
| A158 | 1115.56 | 1116.6 |
| A159 | 991.59 | 992.5 |
| A160 | 949.49 | 950.5 |
| A161 | 1102.53 | 1104 |
| A162 | 1046.58 | 1047.6 |
| A163 | 1037.52 | 1038.8 |
| A164 | 937.49 | 938.5 |
| A165 | 1068.54 | 1069.6 |
| A166 | 1018.47 | 1019.7 |
| A167 | 1059.56 | 1060.4 |
| A168 | 949.49 | 950.9 |
| A169 | 930.54 | 931.5 |
| A170 | 949.49 | 950.8 |
| A171 | 954.54 | 955.5 |
| A172 | 1102.64 | 1103.6 |
| A173/A174 | 1117.63 | 1118.4 |
| A175 | 954.54 | 955.5 |
| A176 | 1081.44 | 1082.4 |
| A177/A178 | 963.56 | 964.4 |
| A179 | 1079.65 | 1080.4 |
| A180/A181 | 1063.66 | 1064.4 |
| A182 | 978.52 | 979.5 |
| A183 | 1047.6 | 1048.8 |
| A184 | 949.49 | 950.7 |
| A185 | 985.58 | 986.9 |
| A186 | 995.58 | 996.3 |
| A187 | 1036.57 | 1037.7 |
| A188 | 1040.55 | 1041.7 |
| A189 | 994.6 | 995.9 |
| A190 | 1036.58 | 1037.8 |
| A191 | 974.54 | 975.8 |
| A192 | 1018.64 | 1019.8 |
| A193 | 977.57 | 978.7 |
| A194 | 1004.62 | 1005.6 |
| A195 | 976.59 | 977.6 |
| A196 | 977.57 | 978.7 |
| A197 | 990.61 | 991.4 |
| A198 | 1017.59 | 1018.9 |
| A199 | 980.58 | 981.5 |
| A200 | 1035.58 | 1036.9 |
| A201 | 1034.63 | 1035.5 |
| A202 | 1003.57 | 1004.5 |
| A203 | 980.58 | 981.5 |
| A204 | 1118.65 | 1119.9 |
| A205 | 979.59 | 980.5 |
| A206 | 1020.62 | 1021.6 |
| A207 | 1038.63 | 1039.5 |
| A208 | 992.53 | 993.8 |
| A209 | 1140.61 | 1141.8 |
| A210 | 1017.53 | 1018.7 |
| A211 | 1064.64 | 1065.8 |
| A212 | 1068.64 | 1069.8 |
| A213 | 977.57 | 978.6 |
| A214 | 981.57 | 982.7 |
| A215 | 991.59 | 992.8 |
| A216 | 977.57 | 978.8 |
| A217 | 979.55 | 980.5 |
| A218 | 1075.66 | 1076.8 |
| A219 | 991.59 | 992.9 |
| A220 | 973.54 | 974.7 |
| A221 | 1031.55 | 1032.9 |
| A222 | 1102.64 | 1104.05 |
| A223 | 1088.62 | 1089.9 |
| A224 | 1030.56 | 1031.4 |
| A225 | 1033.52 | 1034.4 |
| A226 | 995.58 | 996.5 |
| A227 | 1020.62 | 1021.9 |
| A228 | 957.55 | 958.8 |
| A229 | 957.55 | 958.5 |
| A230 | 1049.54 | 1050.8 |
| A231 | 1115.69 | 1117.1 |
| A232 | 1007.58 | 1008.8 |
| A233 | 995.58 | 996.7 |
| A234 | 981.55 | 982.8 |
| A235 | 991.59 | 993 |
| A236 | 965.57 | 966.7 |
| A237 | 1110.66 | 1111.9 |
| A238 | 1117.67 | 1119.1 |
| A239 | 981.57 | 982.7 |
| A240 | 1035.54 | 1036.4 |
| A241 | 1019.55 | 1020.9 |
| A242 | 1033.61 | 1035.5 |
| A243 | 1039.52 | 1040.8 |
| A244 | 979.59 | 980.8 |
| A245 | 979.59 | 980.8 |
| A246 | 979.59 | 980.8 |
| A247 | 979.59 | 980.5 |
| A248 | 965.57 | 966.9 |
| A249 | 965.57 | 966.7 |
| A250 | 985.52 | 986.8 |
| A251 | 1115.69 | 1116.9 |
| A252 | 1053.57 | 1054.7 |
| A253 | 1130.63 | 1132 |
| A254 | 1105.63 | 1106.9 |
| A255 | 1121.62 | 1122.6 |
| A256 | 995.58 | 996.5 |
| A257 | 1029.62 | 1030.6 |
| A258 | 1053.53 | 1056.5 |
| A259 | 1062.59 | 1063.5 |
| A260 | 1058.59 | 1059.5 |
| A261 | 1087.66 | 1089 |
| A262 | 1070.64 | 1071.8 |
| A263 | 1032.62 | 1033.5 |
| A264 | 1044.58 | 1045.5 |
| A265 | 1058.59 | 1059.5 |
| A266 | 1090.6 | 1091.5 |
| A267 | 1002.61 | 1003.5 |
| A268 | 1070.59 | 1071.5 |
| A269 | 1101.67 | 1102.9 |
| A270 | 1098.63 | 1099.2 |
| A271 | 999.59 | 1000.1 |
| A272 | 559.34* | 560.6* |
| A273 | 1101.67 | 1103 |
| A274 | 1035.54 | 1036.8 |
| A275 | 1071.63 | 1072.9 |
| A276 | 1057.61 | 1058.8 |
| A277 | 1102.64 | 1104.1 |
| A278 | 1120.64 | 1121.4 |
| A279 | 1003.48 | 1004 |
| A280 | 1086.55 | 1087.4 |
| A281 | 1088.6 | 1089.5 |
| A282 | 1070.59 | 1071.5 |
| A283 | 1084.61 | 1085.5 |
| A284 | 1062.57 | 1063.5 |
| A285 | 1068.6 | 1069.7 |
| A286 | 993.57 | 994.9 |
| A287 | 1031.55 | 1032.7 |
| A288 | 1017.49 | 1018.6 |
| A289 | 963.52 | 964.6 |
| A290 | 1121.62 | 1122.5 |
| A291 | 1074.61 | 1076.2 |
| A292 | 1052.55 | 1053.8 |
| A293 | 1044.54 | 1045.7 |
| A294 | 995.58 | 996.9 |
| A295 | 1023.54 | 1024.5 |
| A296 | 1112.64 | 1113.8 |
| A297 | 1036.61 | 1037.5 |
| A298 | 993.57 | 994.4 |
| A299 | 1042.56 | 1043.4 |
| A300 | 1056.58 | 1057.4 |
| A301 | 1062.59 | 1063.4 |
| A302 | 1072.61 | 1073.5 |
| A303 | 989.57 | 990.26 |
| A304 | 995.58 | 996.264 |
| A305 | 995.58 | 996.264 |
| A306 | 995.58 | 996.264 |
| A307 | 1049.56 | 1050.24 |
| A308 | 1049.56 | 1050.24 |
| A309 | 1049.56 | 1050.24 |
| A310 | 1049.56 | 1050.24 |
| A311 | 1031.55 | 1032.22 |
| A312 | 1035.54 | 1036.21 |
| A313 | 1099.69 | 1100.6 |
| A314 | 1046.56 | 1047.4 |
| A315 | 1099.58 | 1100.4 |
| A316 | 1070.59 | 1071.5 |
| A317 | 1096.61 | 1097.5 |
| A318 | 1132.72 | 1133.6 |
| A319 | 1035.54 | 1036.7 |
| A320 | 1035.54 | 1036.6 |
| A321 | 1037.56 | 1038.4 |
| A322 | 1102.62 | 1103.6 |
| A323 | 1116.63 | 1117.5 |
| A324 | 1128.72 | 1129.6 |
| A325 | 1004.62 | 1005.9 |
| A326 | 997.56 | 998.5 |
| A327 | 991.59 | 992.7 |
| A328 | 991.59 | 992.7 |
| A329 | 1050.59 | 1051.8 |
| A330 | 1102.62 | 1103.5 |
| A331 | 1102.62 | 1103.5 |
| A332 | 1114.62 | 1115.4 |
| A333 | 1035.54 | 1036.5 |
| A334 | 1035.54 | 1036.5 |
| A335 | 995.56 | 996.7 |
| A336 | 1101.67 | 1102.7 |
| A337 | 1155.65 | 1156.6 |
| A338 | 1088.62 | 1089.6 |
| A339 | 1087.62 | 1088.65 |
| A340 | 1022.6 | 1023.5 |
| A341 | 1076.57 | 1077.5 |
| A342 | 963.56 | 964.6 |
| A343 | 1009.58 | 1010.6 |
| A344 | 1009.58 | 1010.5 |
| A345 | 1114.62 | 1115.5 |
| A346 | 1114.62 | 1115.4 |
| A347 | 993.57 | 994.8 |
| A348 | 993.57 | 994.8 |
| A349 | 1114.62 | 1115.5 |
| A350 | 1005.53 | 1006.7 |
| A351 | 1124.58 | 1125.4 |
| A352 | 1116.63 | 1117.5 |
| A353 | 1114.62 | 1115.3 |
| A354 | 1102.62 | 1103.6 |
| A355 | 1116.63 | 1117.5 |
| A356 | 1124.58 | 1125.5 |
| A357 | 1102.62 | 1103.5 |
| A358 | 1102.62 | 1103.5 |
| A359 | 1032.65 | 1033.6 |
| A360 | 1116.63 | 1117.6 |
| A361 | 1116.63 | 1117.6 |
| A362 | 1044.58 | 1045.4 |
| A363 | 1044.58 | 1045.5 |
| A364 | 976.59 | 977.8 |
| A365 | 994.6 | 995.75 |
| A366 | 1004.62 | 1005.5 |
| A367 | 994.6 | 995.65 |
| A368 | 991.59 | 992.6 |

Note:
values may differ slightly from values found elsewhere in this application due to different measurements and rounding.

### Biological Assays

All compounds herein exhibit an IC₅₀ of 3 μM or less in the H358 (K-Ras G12C) pERK potency assay and/or the MiaPACA-2 (K-Ras G12C) pERK potency assay, each described below.

Potency Assay: pERK

The purpose of this assay is to measure the ability of test compounds to inhibit K-Ras in cells. Activated K-Ras induces increased phosphorylation of ERK at Threonine 202 and Tyrosine 204 (PERK). This procedure measures a decrease in cellular pERK in response to test compounds. The procedure described below in NCI-H₃₅₈ cells is applicable to K-Ras G12C.

Note: This protocol may be executed substituting other cell lines to characterize inhibitors of other RAS variants, including, for example, AsPC-1 (K-Ras G12D), Capan-1 (K-Ras G12V), or NCI-H1355 (K-Ras G13C). An additional protocol regarding an engineered G13C cell line is below.

NCI-H358 cells are grown and maintained using media and procedures recommended by the ATCC. On the day prior to compound addition, cells are plated in 384-well cell culture plates (40 μl/well) and grown overnight in a 37° C., 5% CO₂ incubator. Test compounds are prepared in 10, 3-fold dilutions in DMSO, with a high concentration of 10 mM. On the day of assay, 40 nL of test compound is added to each well of cell culture plate using an Echo550 liquid handler (LabCyte®). Concentrations of test compound are tested in duplicate. After compound addition, cells are incubated 4 hours at 37° C., 5% CO₂. Following incubation, culture medium is removed, and cells are washed once with phosphate buffered saline.

In some experiments, cellular pERK level is determined using the AlphaLISA SureFire Ultra p-ERK1/2 Assay Kit (PerkinElmer). Cells are lysed in 25 μL lysis buffer, with shaking at 600 RPM at room temperature. Lysate (10 μL) is transferred to a 384-well Opti-plate (PerkinElmer) and 5 μL acceptor mix is added. After a 2-hour incubation in the dark, 5 μL donor mix is added, plate is sealed, and incubated 2 hours at room temperature. Signal is read on an Envision plate reader (PerkinElmer) using standard AlphaLISA settings. Analysis of raw data is carried out in Excel (Microsoft) and Prism (GraphPad). Signal is plotted vs. the decadal logarithm of compound concentration, and IC50 is determined by fitting a 4-parameter sigmoidal concentration response model.

In other experiments, cellular pERK is determined by In-Cell Western. Following compound treatment, cells are washed twice with 200 μL tris buffered saline (TBS) and fixed for 15 minutes with 150 μL 4% paraformaldehyde in TBS. Fixed cells are washed 4 times for 5 minutes with TBS containing 0.1% Triton X-100 (TBST) and then blocked with 100 μL Odyssey blocking buffer (LI-COR) for 60 minutes at room temperature. Primary antibody (PERK, CST-4370, Cell Signaling Technology) is diluted 1:200 in blocking buffer, and 50 μL is added to each well and incubated overnight at 4° C. Cells are washed 4 times for 5 minutes with TBST. Secondary antibody (IR-800CW rabbit, LI-COR, diluted 1:800) and DNA stain DRAQ5 (LI-COR, diluted 1:2000) are added and incubated 1-2 hours at room temperature. Cells are washed 4 times for 5 minutes with TBST. Plates are scanned on a Li-COR Odyssey CLx Imager. Analysis of raw data is carried out in Excel (Microsoft) and Prism (GraphPad). Signal is plotted vs. the decadal logarithm of compound concentration, and IC50 is determined by fitting a 4-parameter sigmoidal concentration response model.

The procedure described below in engineered MIA PaCa-2 KRAS G13C A12 cells is applicable to K-Ras G13C.

MIA PaCa-2 KRAS G13C A12 cells are grown and maintained using media and procedures recommended by the ATCC. On the day prior to compound addition, cells are plated in 384-well cell culture plates (8,000 cells/40 μl/well) and grown overnight in a 37° C., 5% CO₂ incubator. Test compounds are prepared in 10, 3-fold dilutions in DMSO, with a high concentration of 10, 1 or 0.1 mM. On the day of assay, 40 nL of test compound is added to each well of cell culture plate using an Echo550 liquid handler (LabCyte®). Concentrations of test compound are tested in duplicate. After compound addition, cells are incubated 4 hours at 37° C., 5% CO2. Following incubation, culture medium is removed, and cells are washed once with phosphate buffered saline.

In some experiments, cellular pERK level is determined using the AlphaLISA SureFire Ultra p-ERK1/2 Assay Kit (PerkinElmer). Cells are lysed in 25 μL lysis buffer, with shaking at 600 RPM at room temperature. Lysate (10 μL) is transferred to a 384-well Opti-plate (PerkinElmer) and 5 μL acceptor mix is added. After a 2-hour incubation in the dark, 5 μL donor mix is added, plate is sealed, and incubated 2 hours at room temperature. Signal is read on an Envision plate reader (PerkinElmer) using standard AlphaLISA settings. Analysis of raw data is carried out in Genedata Screener and Prism (GraphPad). Data are normalized by the following calculation: ((sample signal−average low control)/(average DMSO−average low control))*100. Signal is plotted vs. the decadal logarithm of compound concentration, and IC50 is determined by fitting a 4-parameter sigmoidal concentration response model.

In other experiments, cellular pERK is determined by In-Cell Western. Following compound treatment, cells are washed twice with 200 μL tris buffered saline (TBS) and fixed for 15 minutes with 150 ul 4% paraformaldehyde in TBS. Fixed cells are washed 4 times for 5 minutes with TBS containing 0.1% Triton X-100 (TBST) and then blocked with 100 μL Odyssey blocking buffer (LI-COR) for 60 minutes at room temperature. Primary antibody (PERK, CST-4370, Cell Signaling Technology) is diluted 1:200 in blocking buffer, and 50 μL is added to each well and incubated overnight at 4° C. Cells are washed 4 times for 5 minutes with TBST. Secondary antibody (IR-800CW rabbit, LI-COR, diluted 1:800) and DNA stain DRAQ5 (LI-COR, diluted 1:2000) are added and incubated 1-2 hours at room temperature. Cells are washed 4 times for 5 minutes with TBST. Plates are scanned on a Li-COR Odyssey CLx Imager. Analysis of raw data is carried out in Excel (Microsoft) and Prism (GraphPad). Signal is plotted vs. the decadal logarithm of compound concentration, and IC50 is determined by fitting a 4-parameter sigmoidal concentration response model.

### Determination of Cell Viability in RAS Mutant Cancer Cell Lines

### Protocol: CellTiter-Glo® Cell Viability Assay

Note—The following protocol describes a procedure for monitoring cell viability of K-Ras mutant cancer cell lines in response to a compound of the invention. Other RAS isoforms may be employed, though the number of cells to be seeded will vary based on cell line used.

The purpose of this cellular assay is to determine the effects of test compounds on the proliferation of human cancer cell lines (MIA PaCa-2 KRAS G13C A12 (K-Ras G13C), NCI-H358 (K-Ras G12C), AsPC-1 (K-Ras G12D), and Capan-1 (K-Ras G12V)) over a 5-day treatment period by quantifying the amount of ATP present at endpoint using the CellTiter-Glo® 2.0 Reagent (Promega).

Cells are seeded at 250 cells/well in 40 L of growth medium in 384-well assay plates and incubated overnight in a humidified atmosphere of 5% CO₂ at 37° C. On the day of the assay, 10 mM stock solutions of test compounds are first diluted into 3 mM solutions with 100% DMSO. Well-mixed compound solutions (15 μL) are transferred to the next wells containing 30 μL of 100% DMSO and repeated until a 9-concentration 3-fold serial dilution is made (starting assay concentration of 10 μM). Test compounds (132.5 nL) are directly dispensed into the assay plates containing cells. Alternatively, test compounds are prepared in 9 point, 3-fold dilutions in DMSO, with a high concentration of 10, 1 or 0.1 mM and on the day of the assay, test compounds (40 nL) are directly dispensed into the assay plates containing cells. The plates are shaken for 15 seconds at 300 rpm, centrifuged, and incubated in a humidified atmosphere of 5% CO₂ at 37° C. for 5 days. On day 5, assay plates and their contents are equilibrated to room temperature for approximately 30 minutes. CellTiter-Glo® 2.0 Reagent (25 μL) is added, and plate contents are mixed for 2 minutes on an orbital shaker before incubation at room temperature for 10 minutes. Luminescence is measured using the PerkinElmer Enspire. Data are normalized by the following: (Sample signal/Avg. DMSO)*100. The data are fit using a four-parameter logistic fit.

Disruption of B-Raf Ras-Binding Domain (BRAF^{RBD}) Interaction with K-Ras by Compounds of the Invention (Also Called a FRET Assay or an MOA Assay)

Note—The following protocol describes a procedure for monitoring disruption of K-Ras G13C (GMP-PNP) binding to BRAF^{RBD} by a compound of the invention. This protocol may also be executed substituting other Ras proteins or nucleotides, including K-Ras G12C.

The purpose of this biochemical assay is to measure the ability of test compounds to facilitate ternary complex formation between a nucleotide-loaded K-Ras isoform and Cyclophilin A; the resulting ternary complex disrupts binding to a BRAF^{RBD} construct, inhibiting K-Ras signaling through a RAF effector. Data is reported as IC50 values.

In assay buffer containing 25 mM HEPES pH 7.3, 0.002% Tween20, 0.1% BSA, 100 mM NaCl and 5 mM MgCl₂, tagless Cyclophilin A, His6-K-Ras-GMPPNP, and GST-BRAF^{RBD} are combined in a 384-well assay plate at final concentrations of 25 μM, 12.5 nM and 50 nM, respectively. Compound is present in plate wells as a 10-point 3-fold dilution series starting at a final concentration of 30 μM. After incubation at 25° C. for 3 hours, a mixture of Anti-His Eu-W1024 and anti-GST allophycocyanin is then added to assay sample wells at final concentrations of 10 nM and 50 nM, respectively, and the reaction incubated for an additional 1.5 hours. TR-FRET signal is read on a microplate reader (Ex 320 nm, Em 665/615 nm). Compounds that facilitate disruption of a K-Ras: RAF complex are identified as those eliciting a decrease in the TR-FRET ratio relative to DMSO control wells.

### In Vitro Cell Proliferation Panels

Potency for inhibition of cell growth is assessed at CrownBio using standard methods. Briefly, cell lines are cultured in appropriate medium, and then plated in 3D methylcellulose. Inhibition of cell growth is determined by CellTiter-Glo® after 5 days of culture with increasing concentrations of compounds. Compound potency is reported as the 50% inhibition concentration (absolute IC50). The assay took place over 7 days. On day 1, cells in 2D culture are harvested during logarithmic growth and suspended in culture medium at 1×105 cells/ml. Higher or lower cell densities are used for some cell lines based on prior optimization. 3.5 ml of cell suspension is mixed with 6.5% growth medium with 1% methylcellulose, resulting in a cell suspension in 0.65% methylcellulose. 90 μl of this suspension is distributed in the wells of 2 96-well plates. One plate is used for day 0 reading and 1 plate is used for the end-point experiment. Plates are incubated overnight at 37° C. with 5% CO₂. On day 2, one plate (for to reading) is removed and 10 μl growth medium plus 100 μl CellTiter-Glo® Reagent is added to each well. After mixing and a 10-minute incubation, luminescence is recorded on an EnVision Multi-Label Reader (Perkin Elmer). Compounds in DMSO are diluted in growth medium such that the final, maximum concentration of compound is 10 μM, and serial 4-fold dilutions are performed to generate a 9-point concentration series. 10 μl of compound solution at 10 times final concentration is added to wells of the second plate. Plate is then incubated for 120 hours at 37° C. and 5% CO₂. On day 7 the plates are removed, 100 μl CellTiter-Glo® Reagent is added to each well, and after mixing and a 10-minute incubation, luminescence is recorded on an EnVision Multi-Label Reader (Perkin Elmer). Data is exported to GeneData Screener and modeled with a sigmoidal concentration response model in order to determine the IC50 for compound response.

Not all cell lines with a given RAS mutation may be equally sensitive to a RAS inhibitor targeting that mutation, due to differential expression of efflux transporters, varying dependencies on RAS pathway activation for growth, or other reasons. This has been exemplified by the cell line KYSE-410 which, despite8 having a KRAS G12C mutation, is insensitive to the KRAS G12C(OFF) inhibitor MRTX-849 (Hallin et al., Cancer Discovery 10:54-71 (2020)), and the cell line SW1573, which is insensitive to the KRAS G12C(OFF) inhibitor AMG510 (Canon et al., Nature 575:217-223 (2019)).

### Selective Covalent Modification of G13C

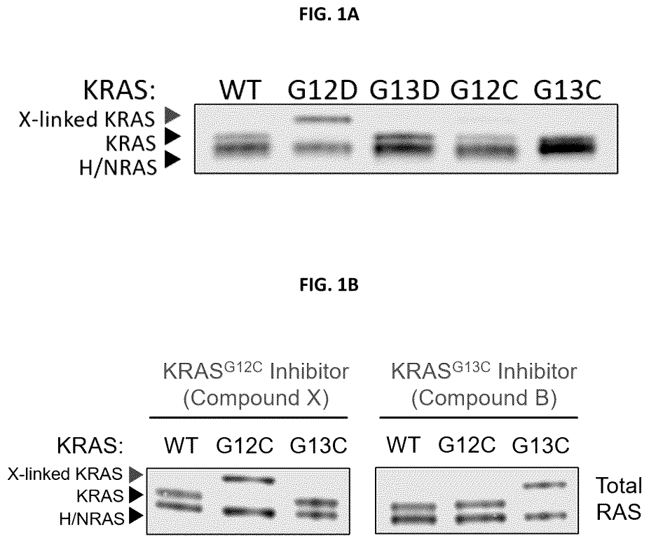

FIG. 1A: NCI-H1975 (WT KRAS), MIA PaCa-2 (KRAS^{G12C/G12C}) and engineered MIA PaCa-2 (KRAS^{G13C/G13C}) cells were treated with 30 nM of Compound A (final concentration DMSO 0.1%) for 1 hour in complete media (DMEM+10% FBS+1% PenStrep). After the treatment period, cells were lysed in NP-40 lysis buffer supplemented with 1× Halt protease and phosphatase inhibitor (Thermo). Proteins in lysate were separated by SDS-PAGE (NuPage 12% Bis-Tris gel, Invitrogen) and transferred to a nitrocellulose membrane. Western blot analysis was performed by probing the membrane with an anti-RAS antibody (Abcam 108602) and detection of the RAS protein was performed using the LiCor Odyssey CLx.

FIG. 1B: NCI-H1975 (WT KRAS), MIA PaCa-2 (KRAS^{G12C/G12C}) and MOR (KRAS^{G13C/G13C}) cells were treated with 50 nM of Compound X, a KRAS^{G12C} inhibitor from WO 2021/091982 (A647), and Compound B, a compound of the present invention (final concentration DMSO 0.1%) for 2 hours in complete media (RPMI-1640+10% FBS+1% PenStrep for NCI-H1975 and MOR; DMEM+10% FBS+1% PenStrep for MIA PaCa-2). After the treatment period, cells were lysed in NP-40 lysis buffer supplemented with 1× Halt protease and phosphatase inhibitor (Thermo). Proteins in lysate were separated by SDS-PAGE (NuPage 12% Bis-Tris gel, Invitrogen) and transferred to a nitrocellulose membrane. Western blot analysis was performed by probing the membrane with an anti-RAS antibody (Abcam 108602) and detection of the RAS protein was performed using the LiCor Odyssey CLx.

### Single Dose PK/PD In Vivo Inhibition of KRAS^{G13C} Using Compound A, a Compound of the Present Invention

### Methods:

The NCI-H1734 KRASG13C/wt cell line-derived xenograft model of human non-small cell lung cancer was used for a single-dose pharmacokinetics (PK)/pharmacodynamics (PD) study. Female NOD SCID mice (6-8 weeks old) were subcutaneously implanted with NCI-H1734 tumor cells (1×10⁷ cells/mouse) using Matrigel (1:1 ratio with culture medium) into the right flank. Once tumors reached approximately 400-600 mm³ range as measured by caliper, mice were randomized into groups to start the administration of Compound A or vehicle. Compound A was administered by oral gavage (po) at 100 mg/kg. The treatment groups with sample collections at various time points after dosing were summarized in Table 4 below. Tumor samples were collected to assess RAS/ERK pathway modulation by measuring the mRNA level of human DUSP6 (PD) in qPCR assay. Plasma samples were collected to assess unbound plasma concentration (PK) by LC-MS bioanalytical assay.

TABLE 4
Summary of treatment groups, doses, and time
points for single-dose pharmacokinetics/pharmacodynamics
study using NCI-H1734 tumors.

| | | | PD, n = 3/time | PK, n = 3/time |
|---|---|---|---|---|
| Compound/group | Dose/Regimen | | point | point |
| Vehicle control | 10 | ml/kg po | 24 h | 24 h |
| Compound A | 100 | mg/kg po | 3 h, 8 h, 24 h | 0.5 h, 1 h, 3 h, |
| | | | | 8 h, 24 h |

### Results:

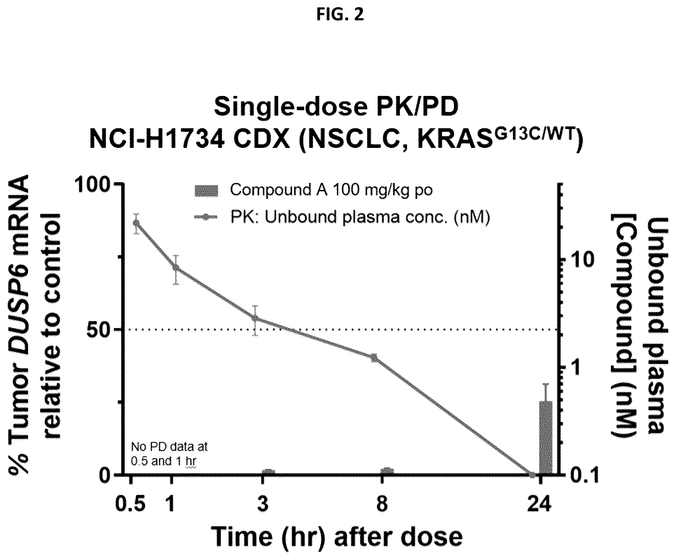

In FIG. 2, Compound A led to inhibition of DUSP6 mRNA levels in NCI-H1734 xenografted tumors at 3, 8 and 24 hours after dosing. Potent inhibition of DUSP6 levels was observed at greater than 95% inhibition relative to control at 3 and 8-hour timepoints. At the 24-hour timepoint where unbound Compound A plasma concentration was undetectable, inhibition of DUSP6 level was still maintained with approximately 75% inhibition relative to control. Overall, Compound A potently inhibited DUSP6 mRNA levels, indicating strong suppression of RAS/ERK signaling in the NCI-H₁₇₃₄ KRASG13C/wt cell line-derived xenograft model of human non-small cell lung cancer.

### Tumor Regressions in Models of KRAS^{G13C} Cancers Using Compound A, a Compound of the Present Invention

### Methods:

The NCI-H1734 KRAS^{G13C/wt} cell line-derived xenograft model of human non-small cell lung cancer was used for an efficacy study. Female NOD SCID mice (6-8 weeks old) were subcutaneously implanted with NCI-H1734 tumor cells (1×10⁷ cells/mouse) using Matrigel (1:1 ratio with culture medium) into the right flank. Once tumor volumes reached approximately 150-250 mm³ range as measured by caliper, mice were randomized into treatment groups to start the administration of Compound A or vehicle. Compound A was administered by oral gavage (po) at 100 mg/kg. Body weight and tumor volume (using caliper) was measured twice weekly until study endpoints. Tumor volume (mm³) was calculated based on the formula: width²×length×0.5.

### Results:

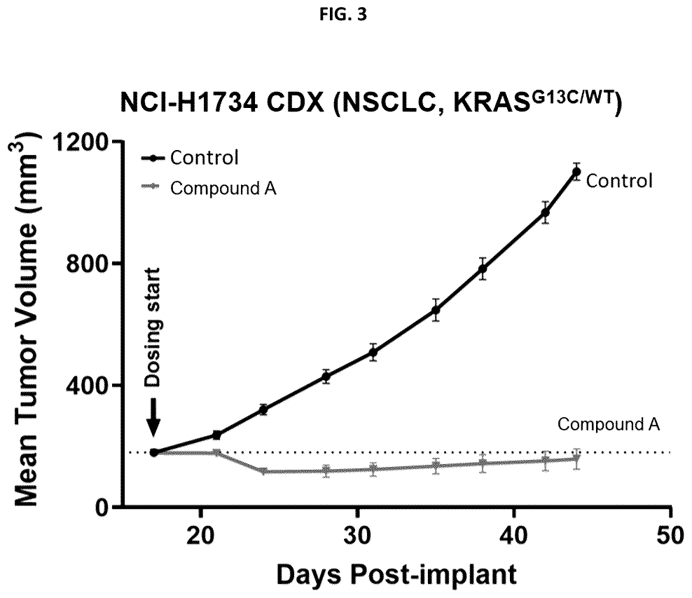

FIG. 3 shows Compound A dosed at 100 mg/kg by daily oral gavage led to tumor regression in the NCI-H1734 KRAS^{G13C/wt} cell line-derived xenograft model of human non-small cell lung cancer. At the end of the 28-day efficacy study, a mean tumor regression of 11% was achieved.

### Methods:

The ST2822B KRAS^{G13C/wt} patient-derived xenograft model of human non-small cell lung cancer was used for an efficacy study. ST2822B tumor fragments were harvested from host mice and implanted into female athymic nude (immune-deficient) mice 6-12 weeks old. Once tumor volumes reached approximately 150-300 mm³ range as measured by caliper, mice were randomized into groups of five mice each to start the administration of Compound A or vehicle. Compound A was administered by oral gavage (po) at 100 mg/kg. Body weight and tumor volume (using caliper) was measured twice weekly until study endpoints. Tumor volume (mm³) was calculated based on the formula: width²×length×0.5.

### Results:

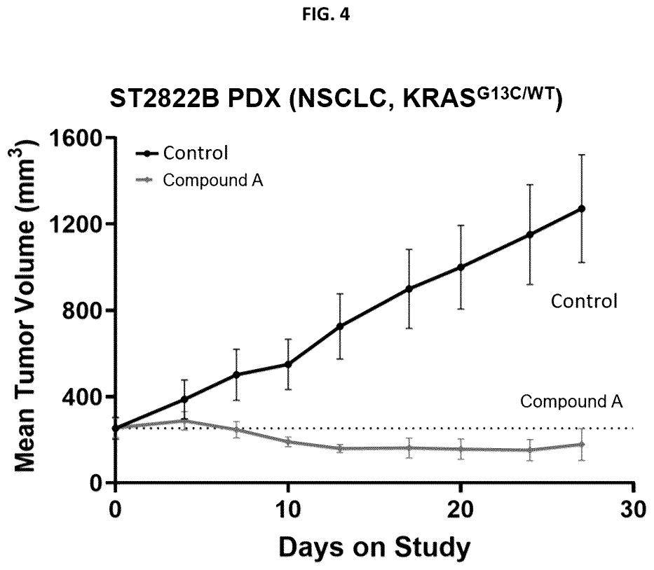

FIG. 4 shows Compound A dosed at 100 mg/kg by daily oral gavage led to tumor regression in the ST2822B KRAS^{G13C/wt} patient-derived xenograft model of human non-small cell lung cancer. At the end of the 28-day efficacy study, a mean tumor regression of 30% was achieved.

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features set forth herein.

All publications, patents and patent applications are herein incorporated by reference in their entirety to the same extent as if each individual publication, patent, or patent application was specifically and individually indicated to be incorporated by reference in its entirety.

LENGTHY TABLES
The patent application contains a lengthy table section. A copy of the table is available in electronic form from the USPTO web site (https://seqdata.uspto.gov/?pageRequest=docDetail&DocID=US20250197423A1). An electronic copy of the table will also be available from the USPTO upon request and payment of the fee set forth in 37 CFR 1.19(b)(3).

1-21. (canceled)

22. A compound, selected from the group consisting of: 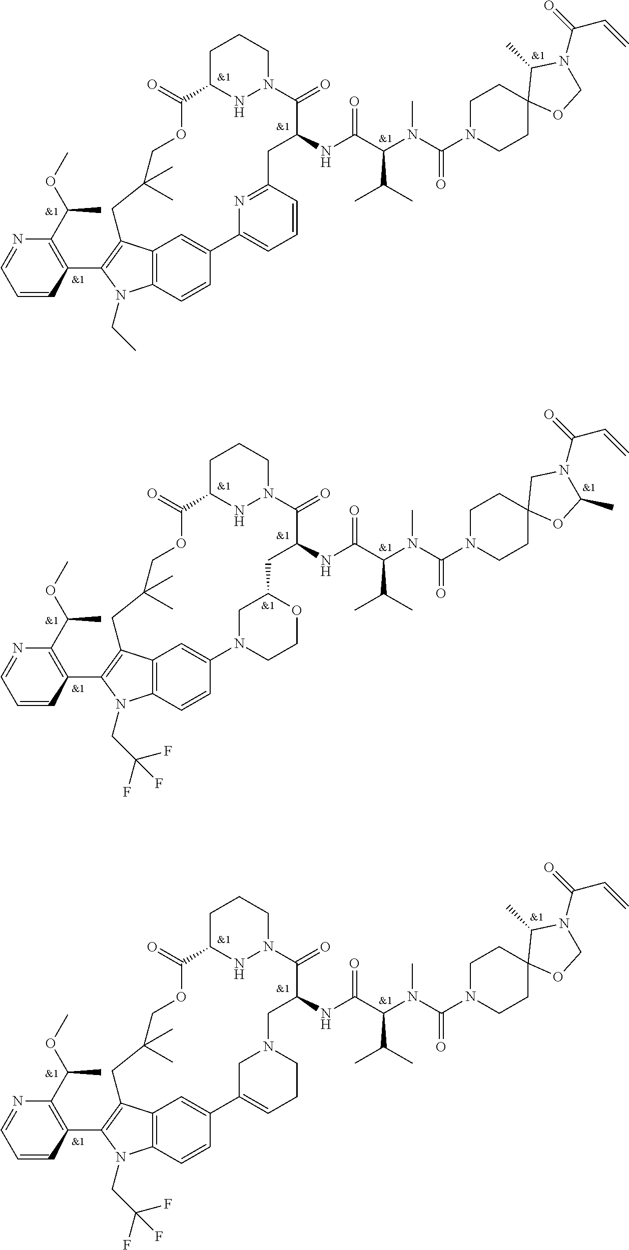 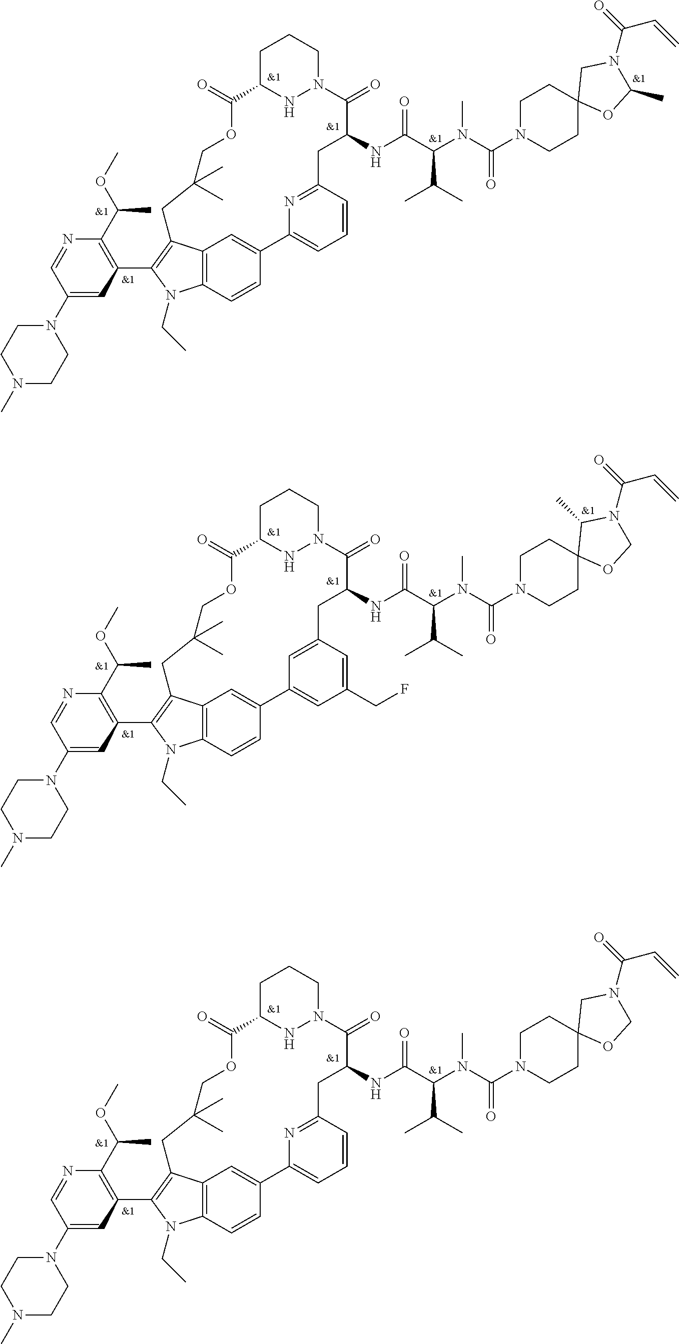 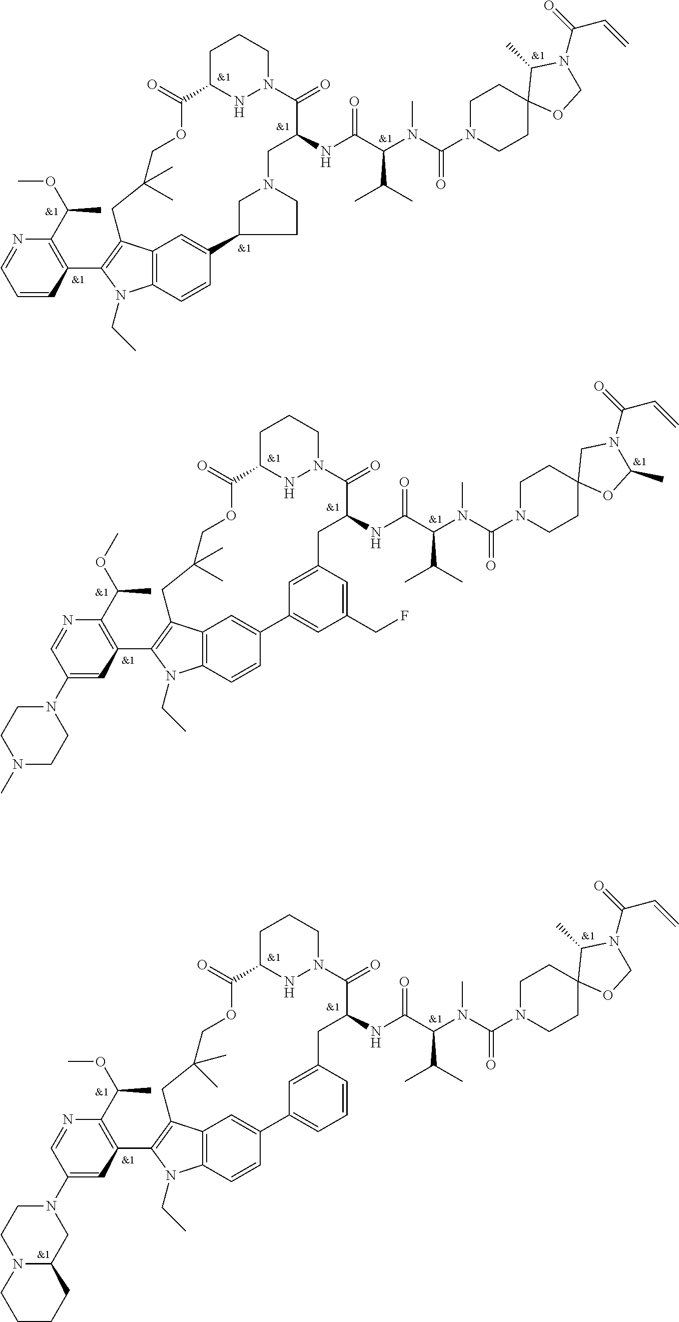 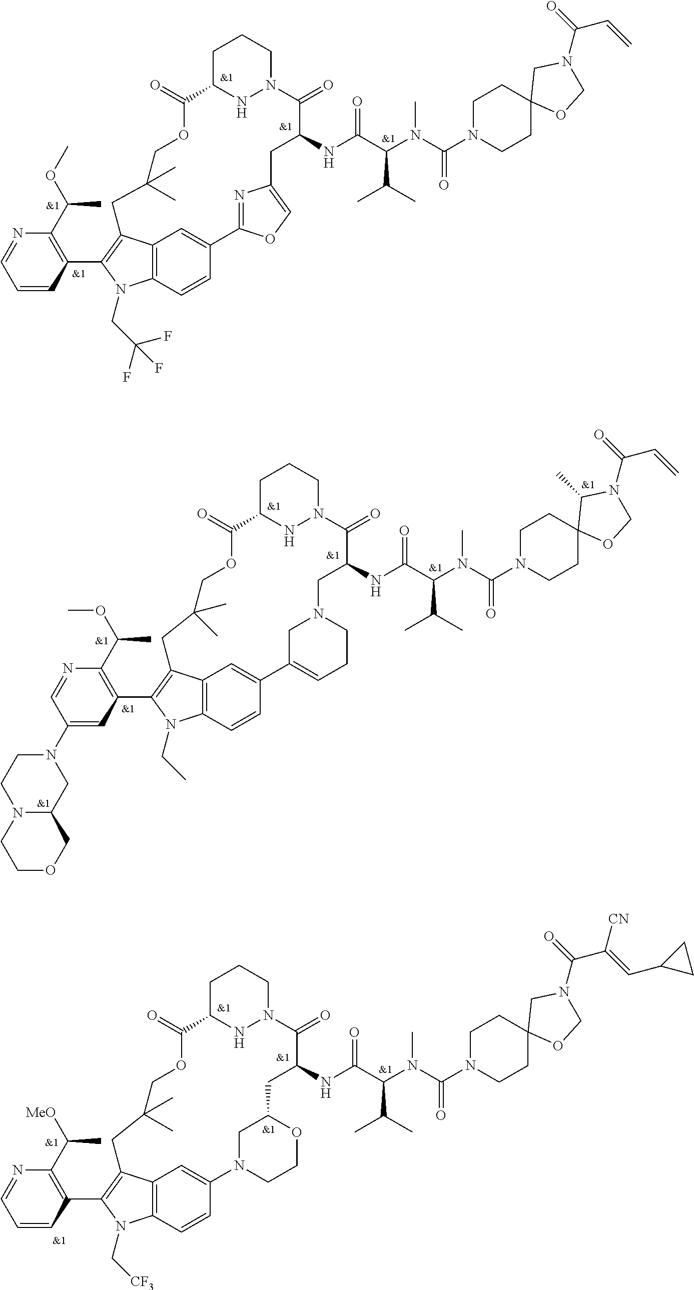 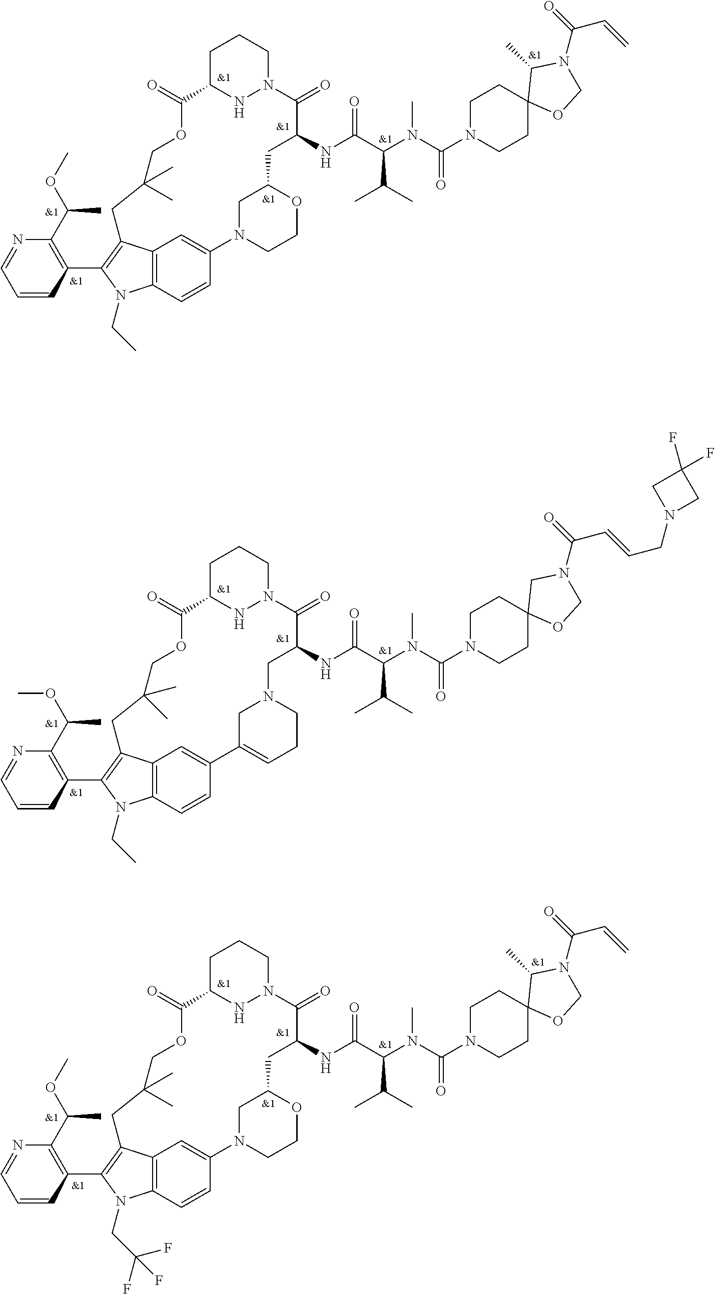 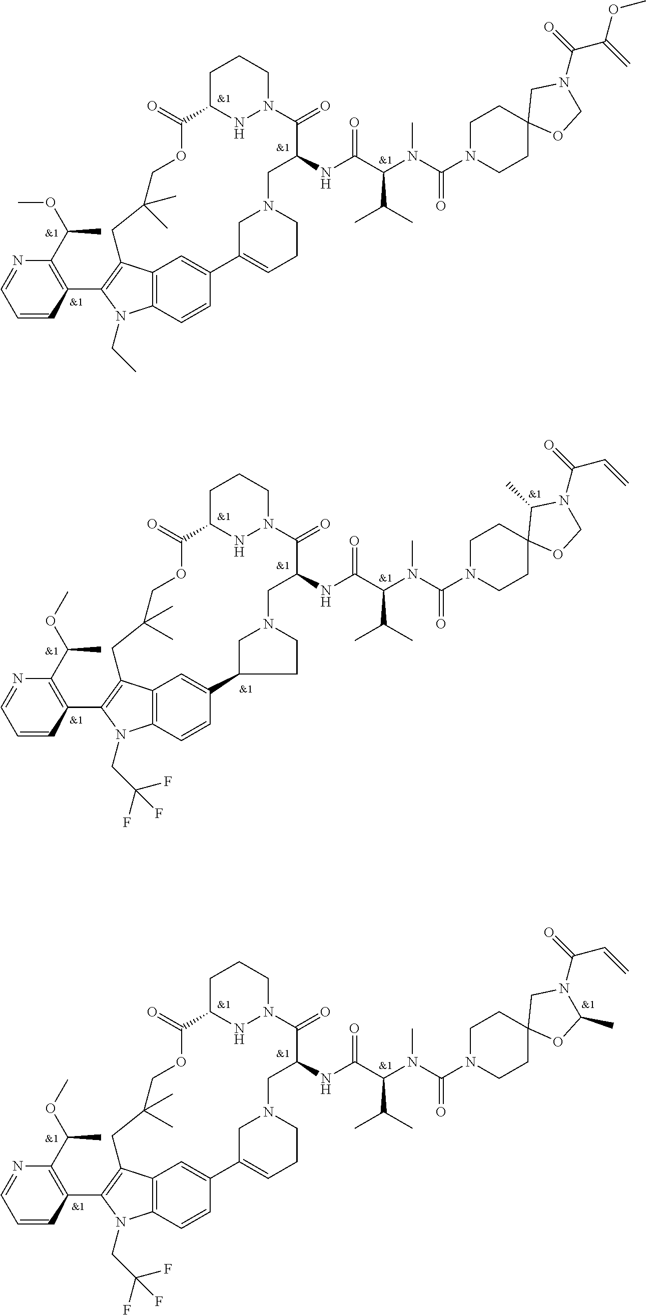 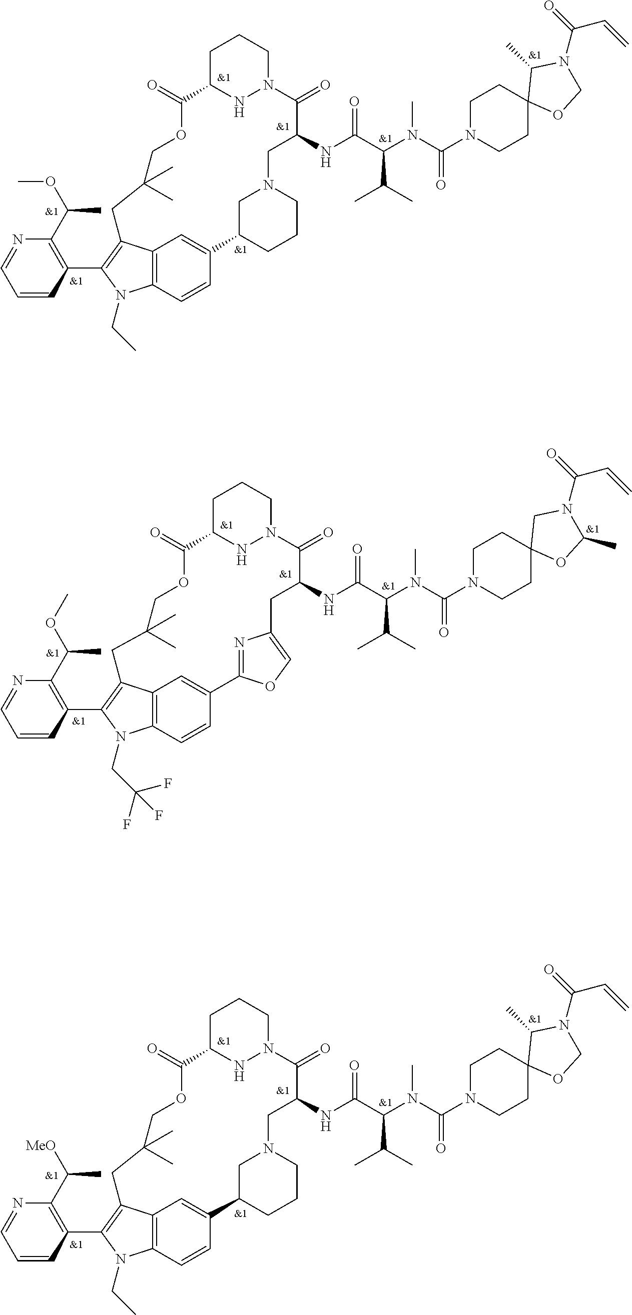 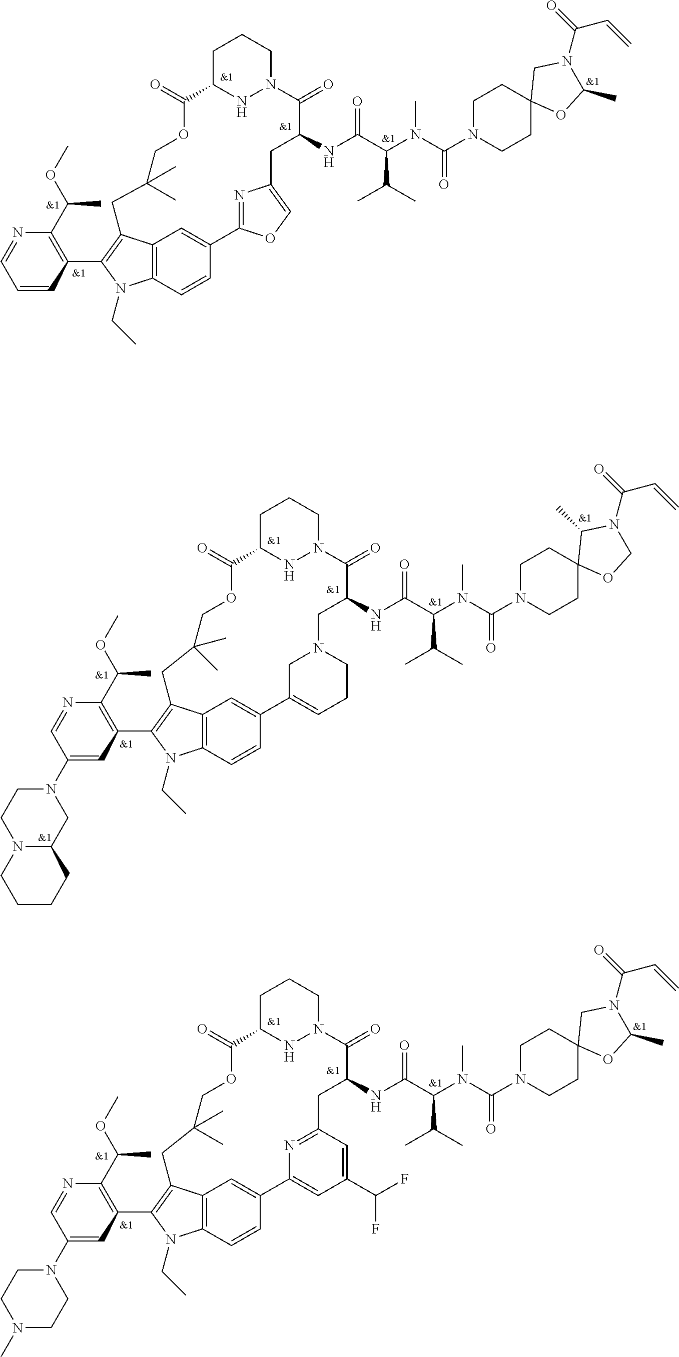 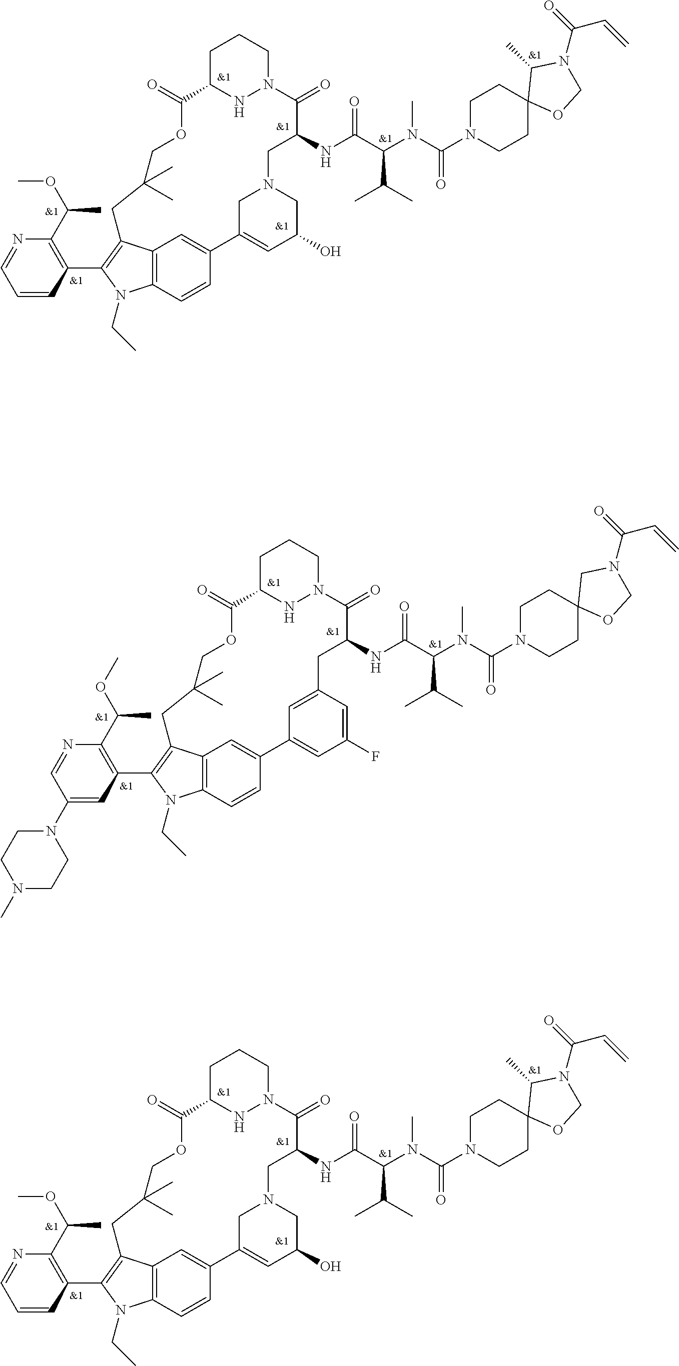 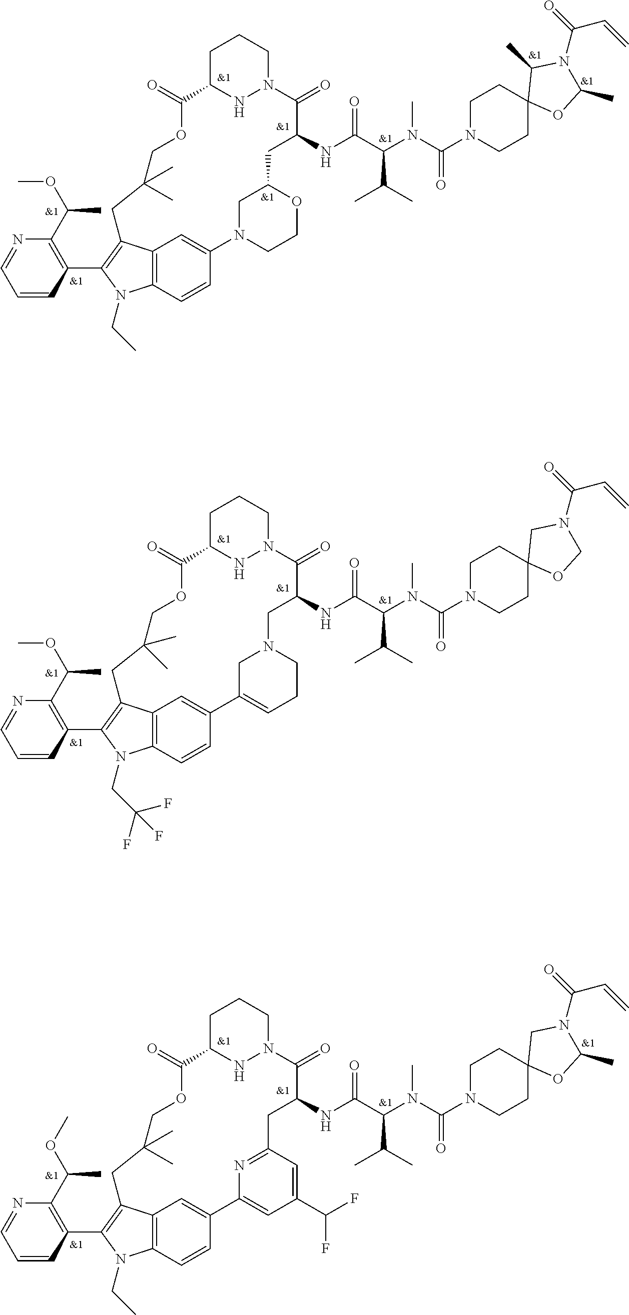 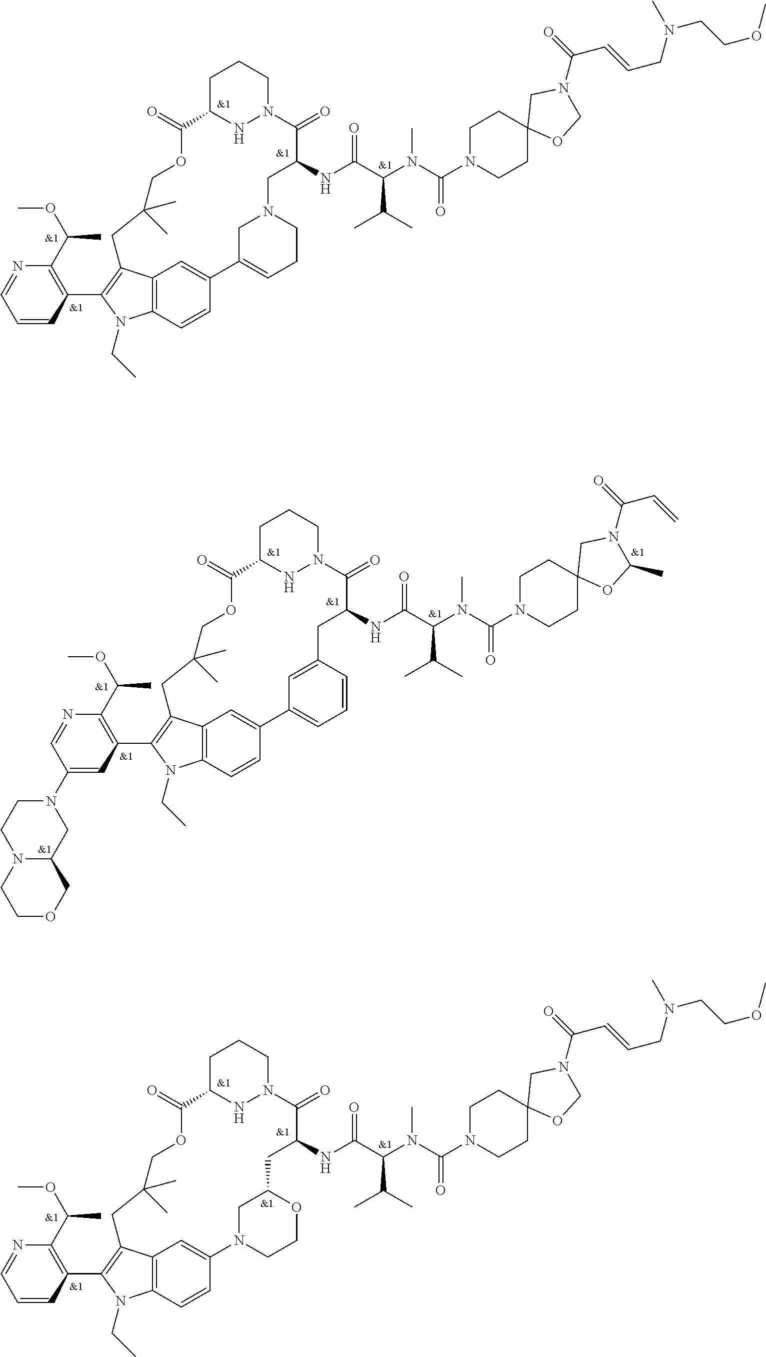 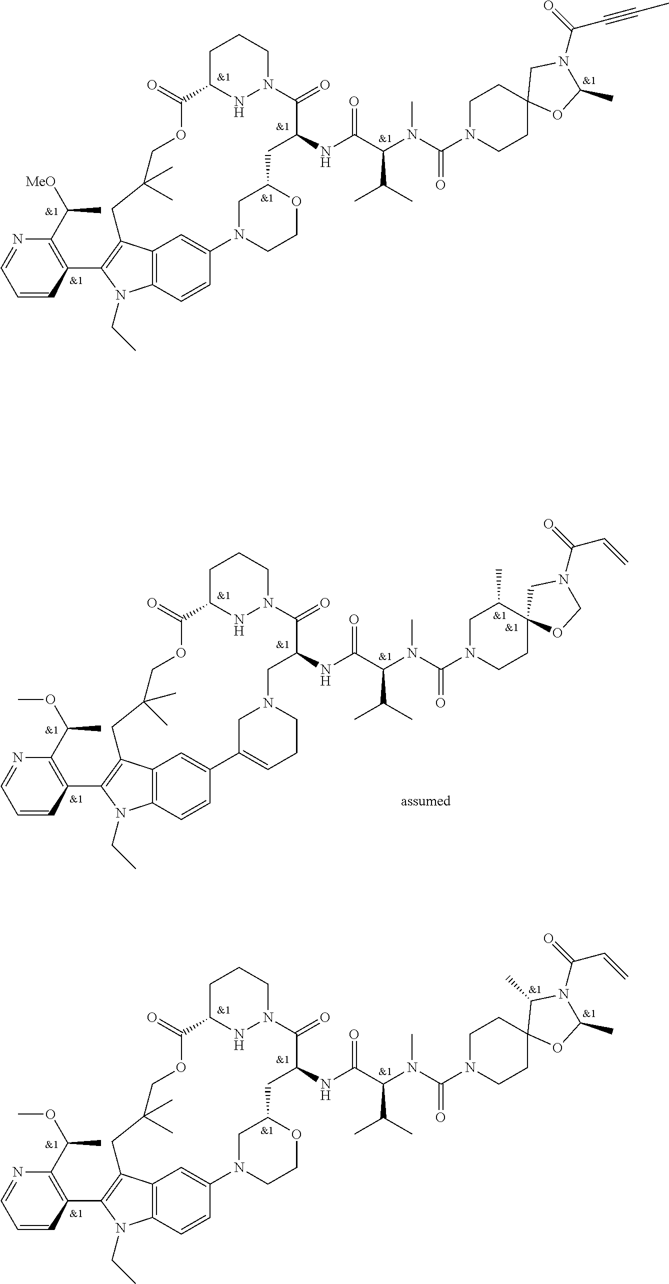 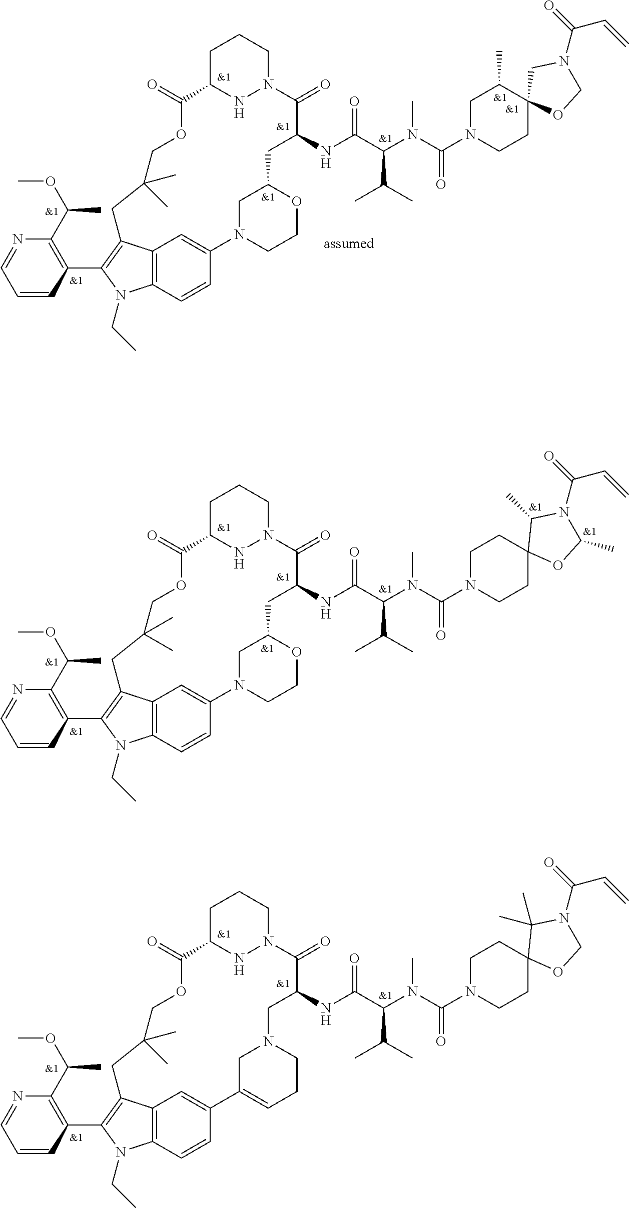 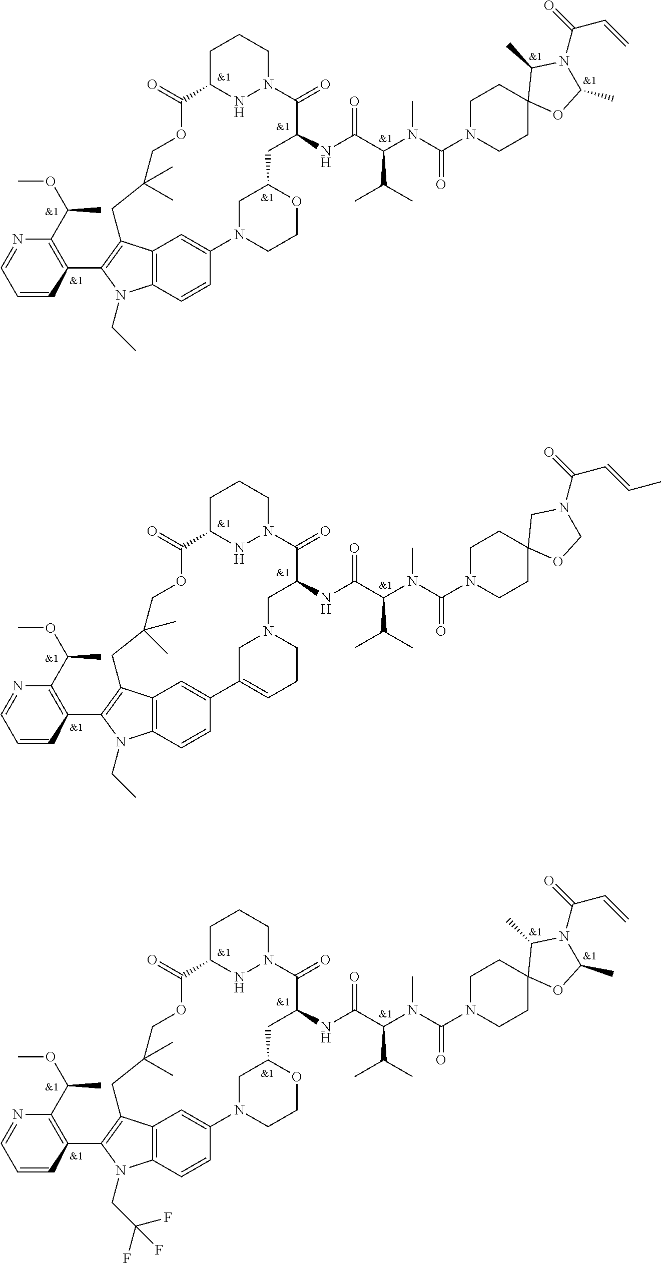 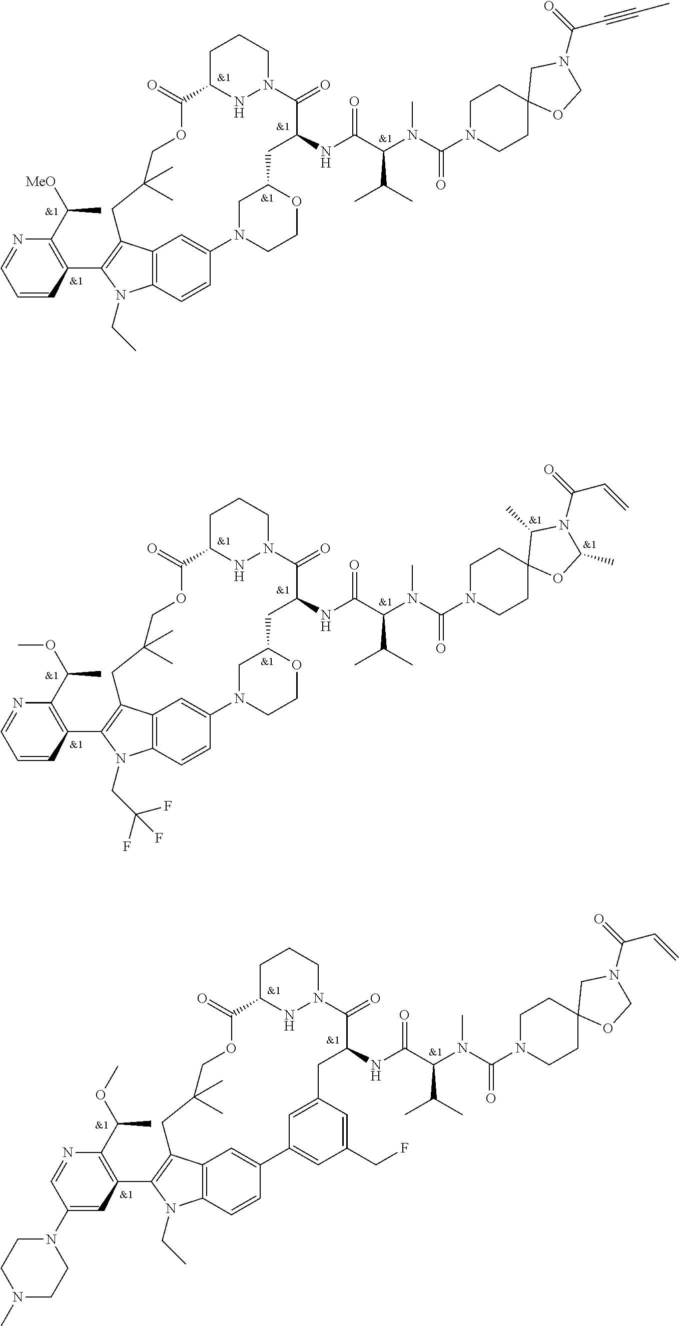 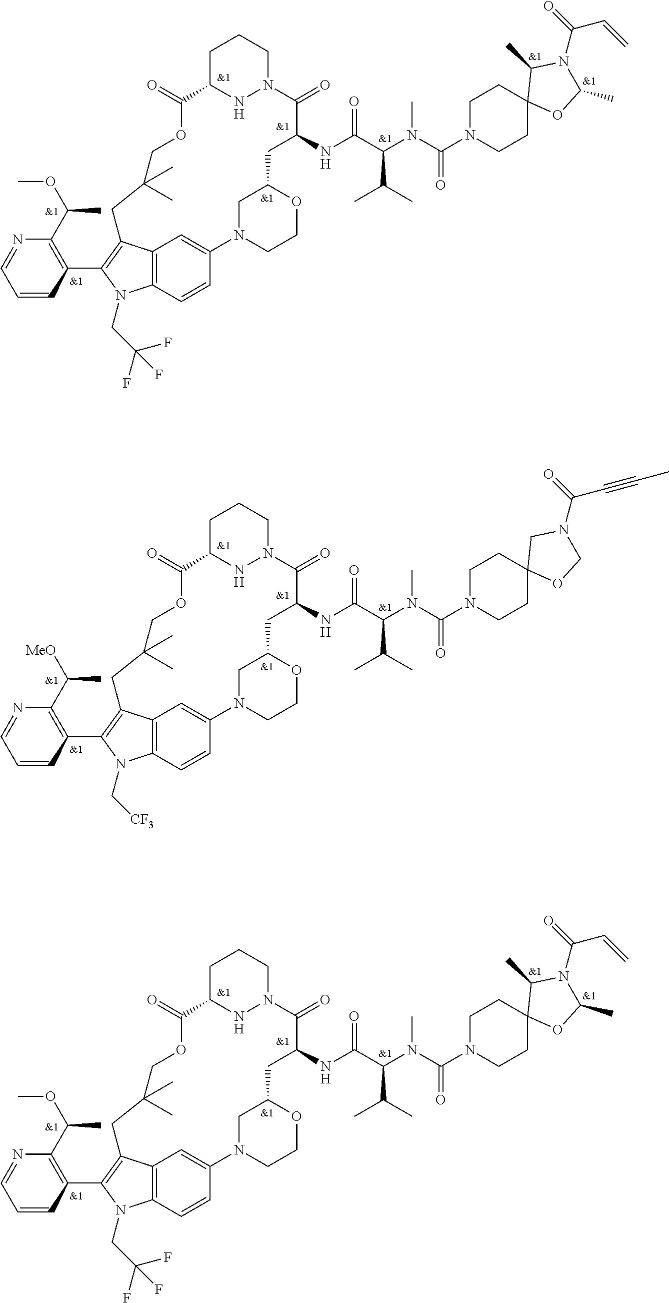 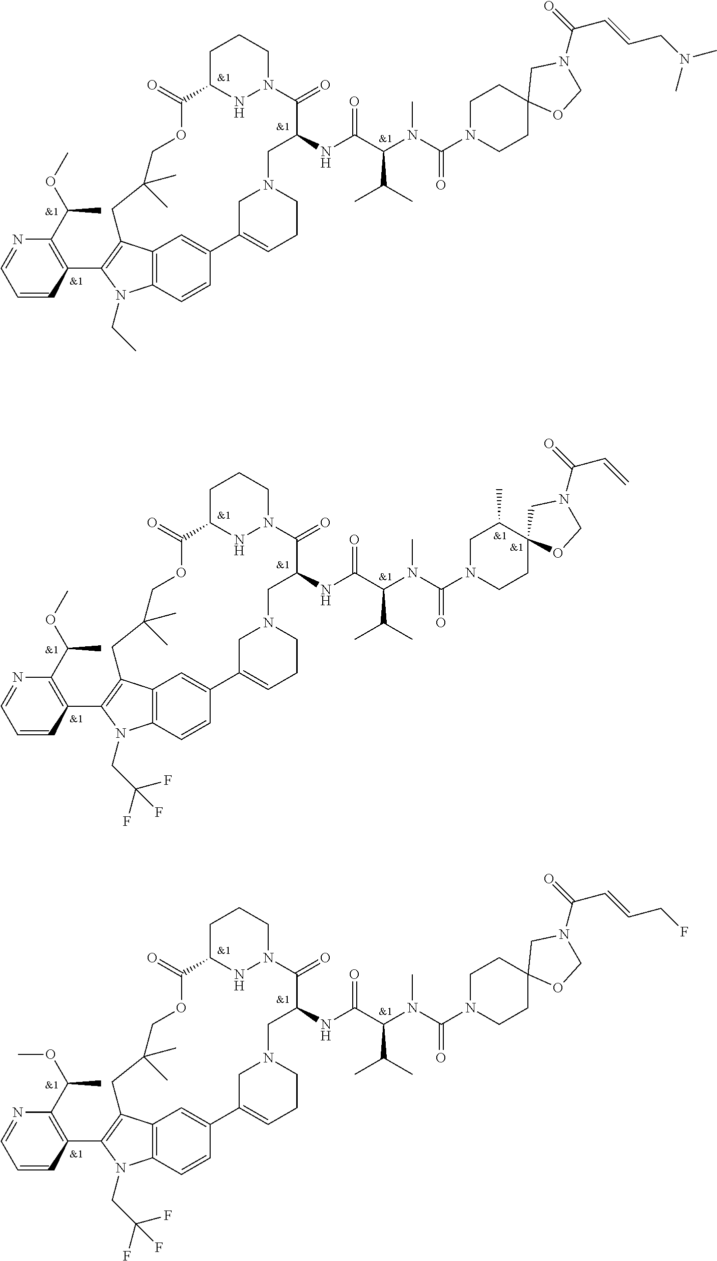 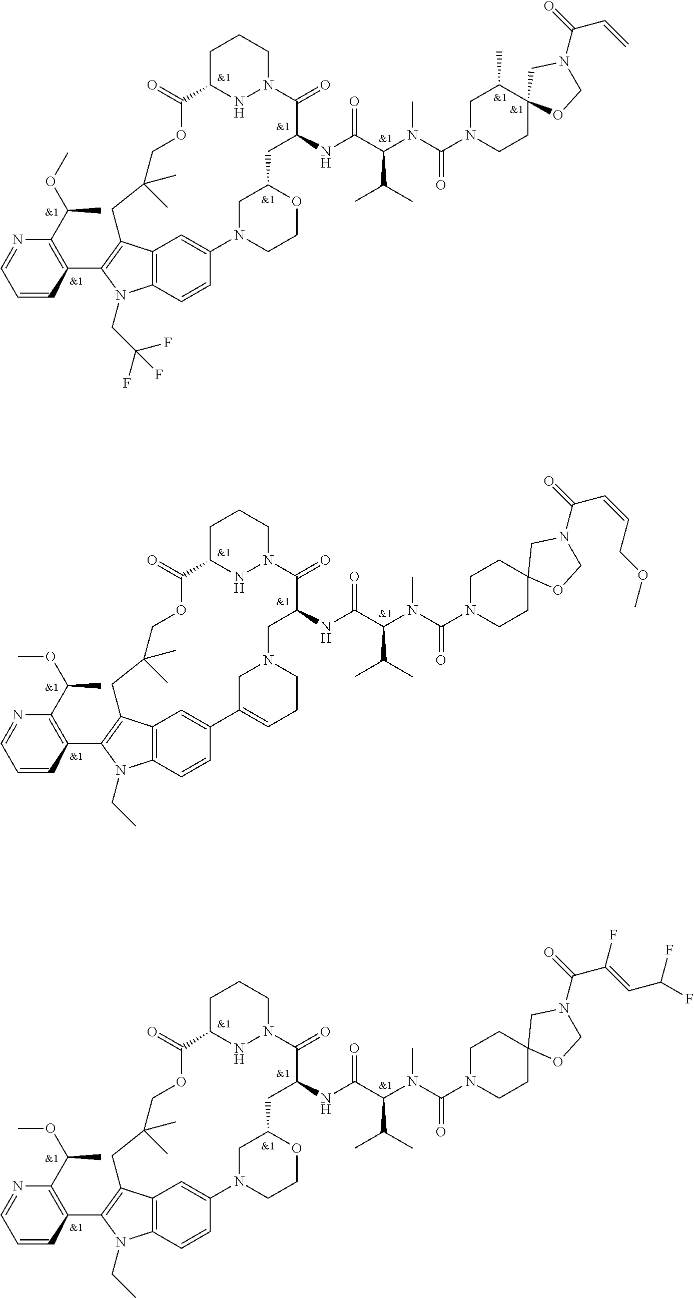 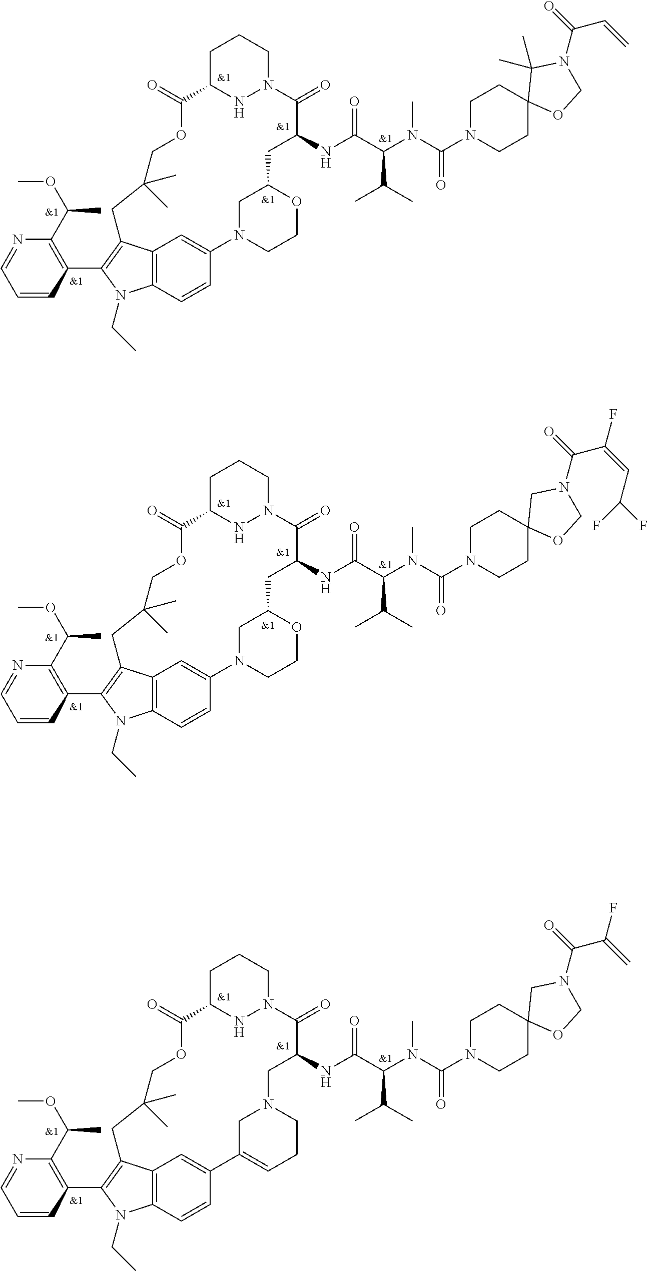 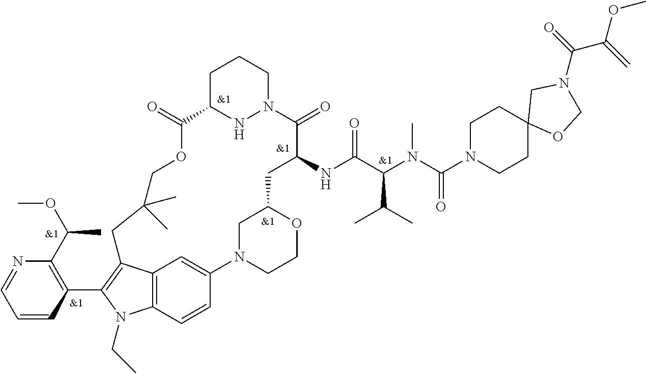 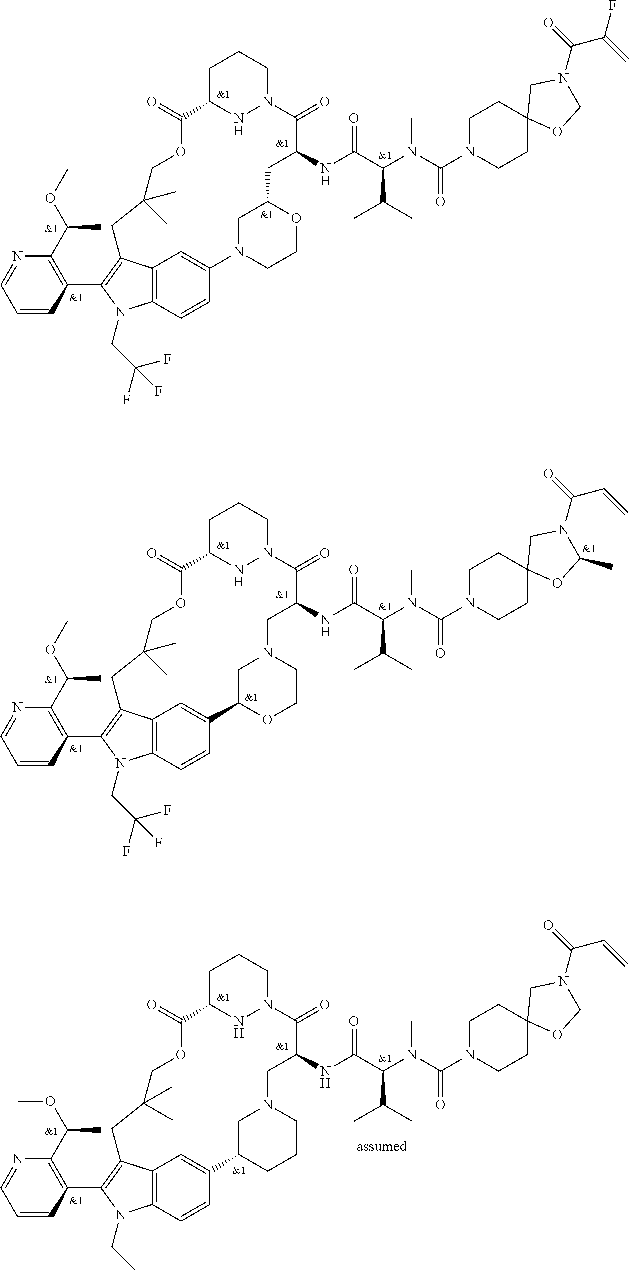 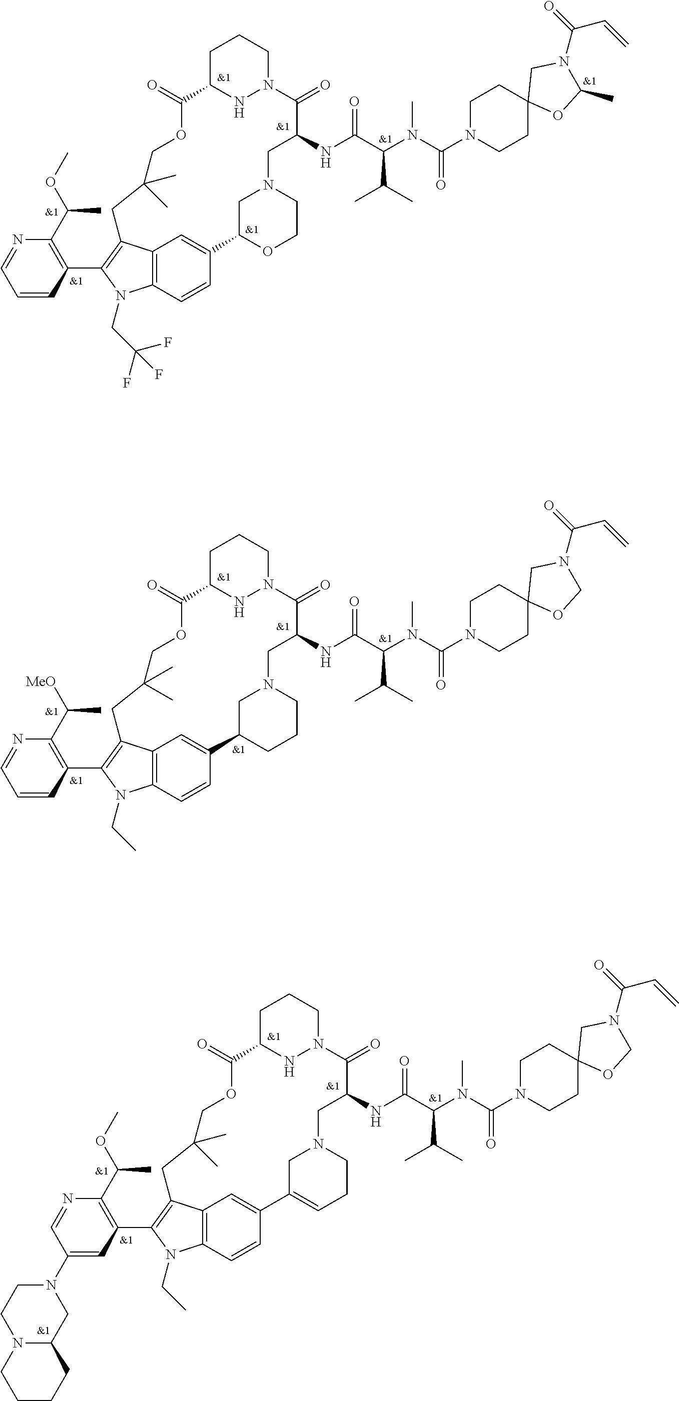 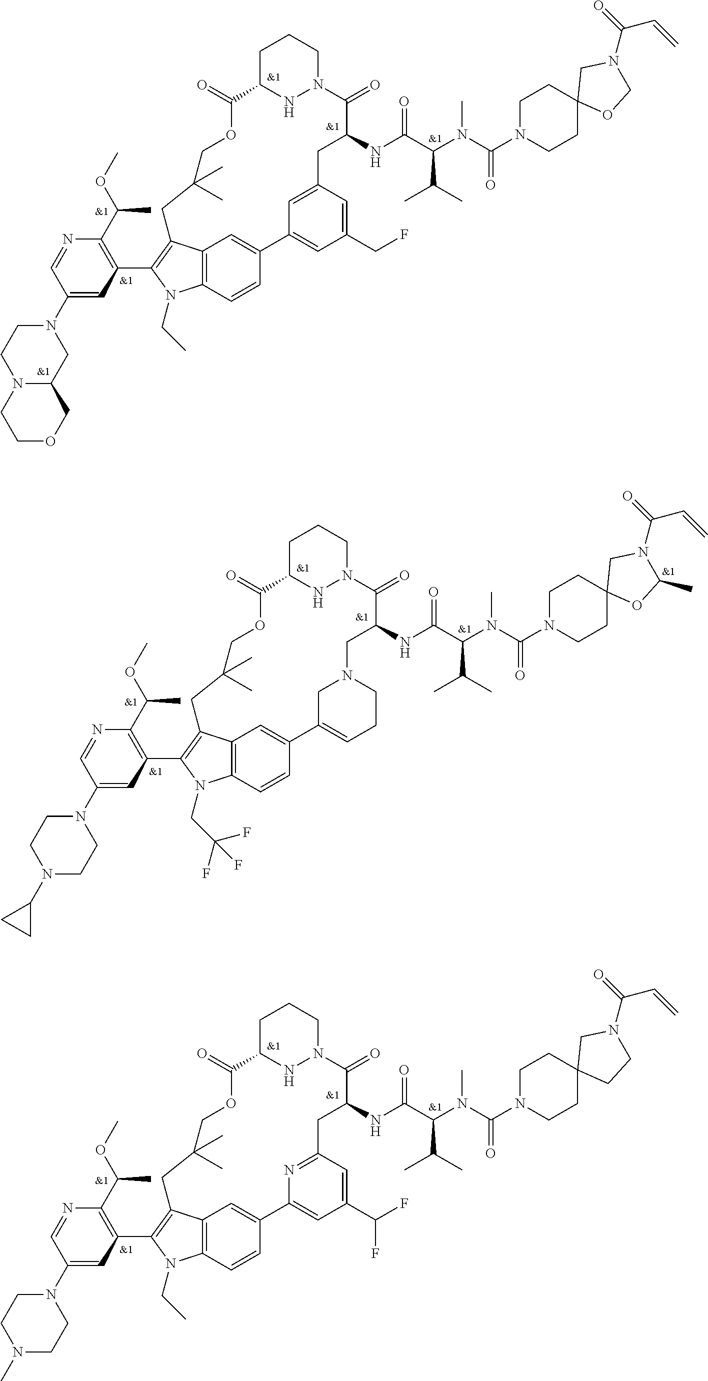 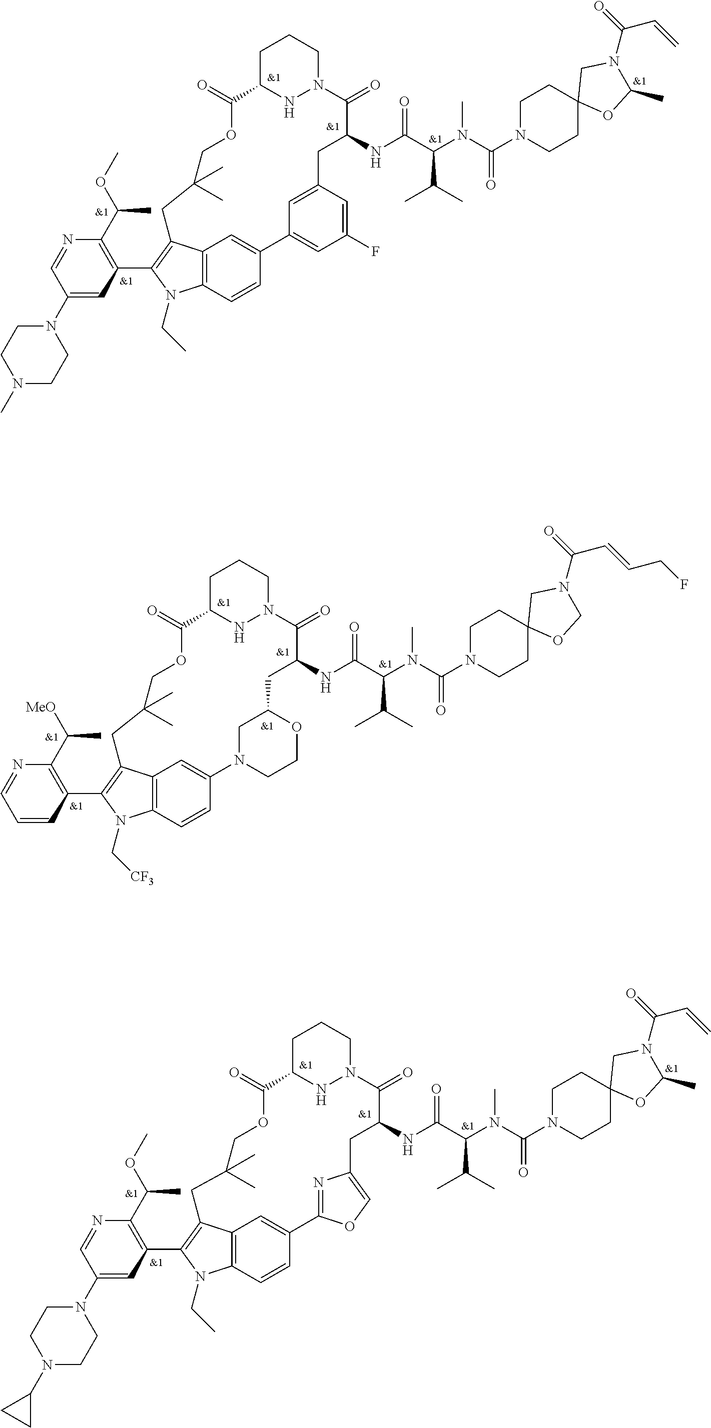 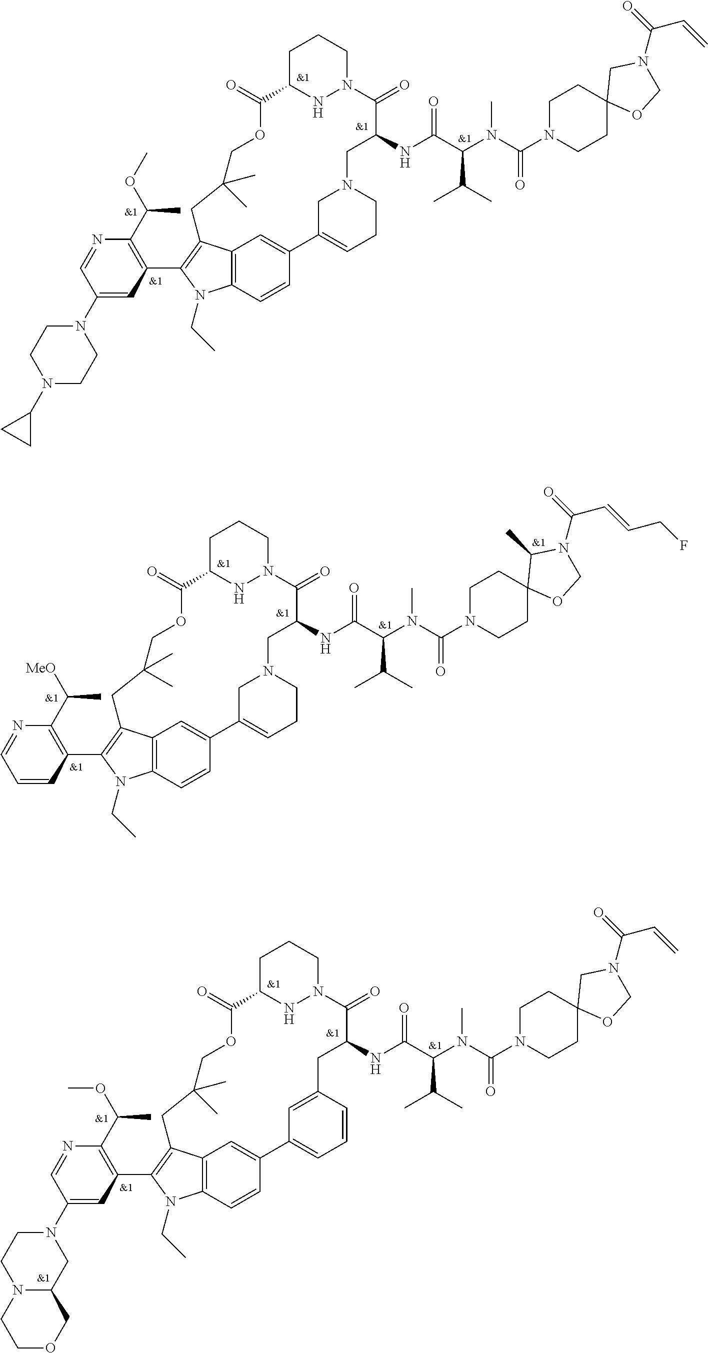 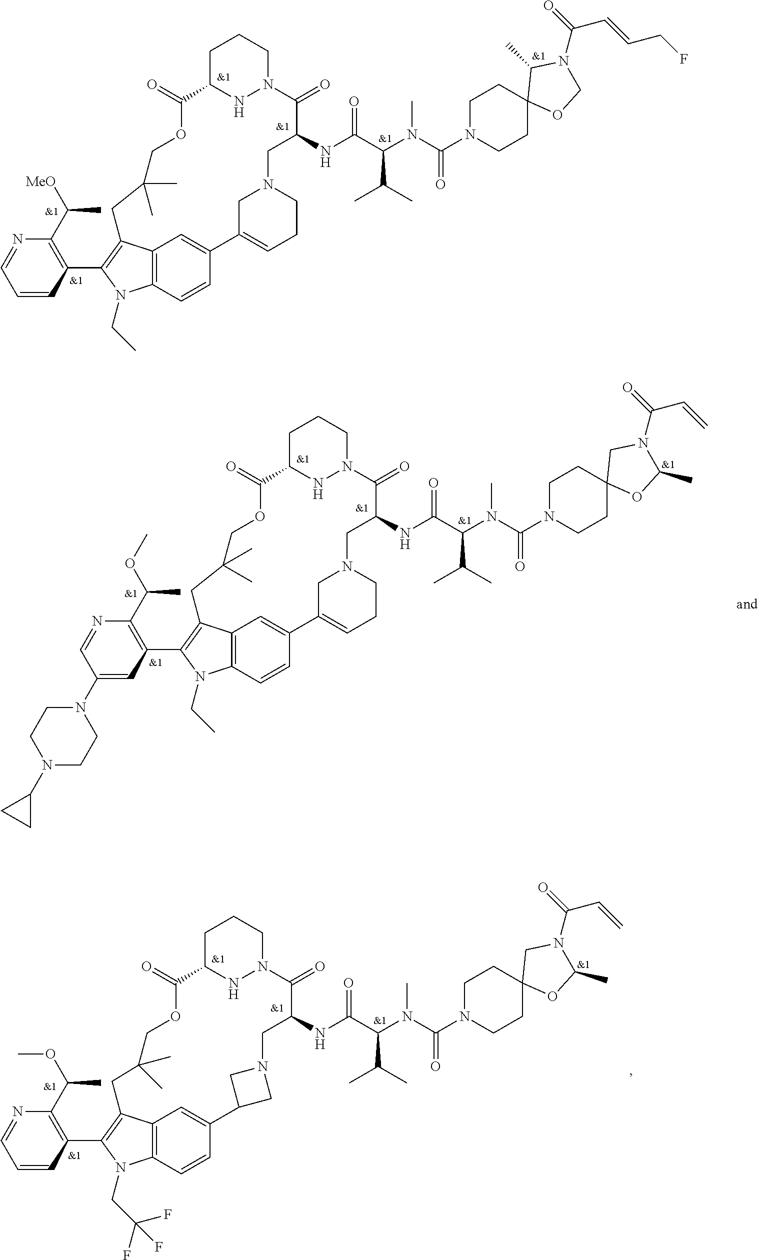
or a pharmaceutically acceptable salt thereof.

23. A pharmaceutical composition comprising a compound of claim 22, or a pharmaceutically acceptable salt thereof, and a pharmaceutically acceptable excipient.

24. (canceled)

25. A method of treating cancer in a subject in need thereof, the method comprising administering to the subject a therapeutically effective amount of a compound of claim 22, or a pharmaceutically acceptable salt thereof.

26. The method of claim 25, wherein the cancer is pancreatic cancer, colorectal cancer, non-small cell lung cancer, or endometrial cancer.

27. The method of claim 25, wherein the cancer comprises a Ras mutation.

28. A method of treating a Ras protein-related disorder in a subject in need thereof, the method comprising administering to the subject a therapeutically effective amount of a compound of claim 22, or a pharmaceutically acceptable salt thereof.

29. A method of inhibiting a Ras protein in a cell, the method comprising contacting the cell with an effective amount of a compound of claim 22, or a pharmaceutically acceptable salt thereof.
